SEQUENCE LISTING (688 sequences)

SEQ ID NO: 1 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaataaaaactaccaaccagttatcatttctctgtgtaatatctgaaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 2 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcgcagaatatcgatatcttcacacagatcctgcacacacccagacagctggcgctcacacattcaccgttggcctgcctctgttcaccctcc

SEQ ID NO: 3 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacaccgaatagcacctatagctcacacattcaccgttggcctgcctctgttcaccctcc

SEQ ID NO: 4 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaacttacctactaagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 5 - skipped

SEQ ID NO: 6 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagtaattcgtccgatagattgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 7 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggaattcagacgacaaaccattcttggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 8 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcttctaagtccaattcacgacaagagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 9 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagcgttgaagcttggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 10 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgagtcgtcagactcaattattactggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 11 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaatccctagcgatcgaagttgataaaacctaagttttgtaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 12 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgcgccttcataaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 13 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatatctcgctaataggagtaagatacatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 14 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcattctgctgcaagacctatactataactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 15 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataccacattgctatagtgctgtataatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 16 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggttgcgtaccagaatatttaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 17 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagacttggtcactatcacgtatataccatcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 18 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggacgagttcgataatacactgttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 19 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgaatactggtgcttcaattacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 20 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgcccgatagaaagaataatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 21 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggtgtctgtataaagataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 22 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttgttaagcatactaaactgtcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 23 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagtttcgagcgacgcttaatattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 24 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccatagatagtacgggttccatactgcccctgtgataaactgtggtcactggctgtggcagcaactattataagatgctctgaaaactcttcagacactgaggggcaccagaggagcagactacaaga

SEQ ID NO: 25 - DNA, 2000 bp, synthetic construct
  source 1..2000: mol_type = other DNA
tctccttcaccttccaccatgagtaaaagcttcttgaggcctcttacctactaggttaaggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 26 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaacactcgaattcagaaaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 27 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgattctagccttacagcctaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 28 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgactctacggaagtagcttgtttaaaacctatagtctcttcggagtcgttctactagtacaaaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 29 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttgagtaaactaactttcaaccgctcttcgtggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 30 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgcttaaacaccgttttgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 31 - DNA, 66 bp, Homo sapiens
  source 1..66: mol_type = other DNA
atgtgtcaccagcagctcgttatatcctggtttagtttggtgtttctcgcttcacccctggtggca

SEQ ID NO: 32 - DNA, 66 bp, synthetic construct
  source 1..66: mol_type = other DNA
atgtgccatcagcaactcgtcatctcctggttctcccttgtgttcctcgcttcccctctggtcgcc

SEQ ID NO: 33 - DNA, 42 bp, synthetic construct
  source 1..42: mol_type = other DNA
atgcaactgctgtcatgtatcgcactcatcctggcgctggta

SEQ ID NO: 34 - DNA, 60 bp, Homo sapiens
  source 1..60: mol_type = other DNA
atgtatcggatgcaacttttgagctgcatcgcattgtctctggcgctggtgacaaattcc

SEQ ID NO: 35 - DNA, 45 bp, Homo sapiens
  source 1..45: mol_type = other DNA
atgaatctcttgctcatacttacgtttgtcgctgctgccgttgcg

SEQ ID NO: 36 - DNA, 51 bp, Gaussia sp.
  source 1..51: mol_type = other DNA
atgggcgtgaaggtcttgtttgcccttatctgcatagctgttgcggaggcg

SEQ ID NO: 37 - DNA, 72 bp, Homo sapiens
  source 1..72: mol_type = other DNA
atgccgatggggagccttcaacctttggcaacgctttatcttctggggatgttggttgctagttgccttggg

SEQ ID NO: 38 - DNA, 63 bp, Mus musculus
  source 1..63: mol_type = other DNA
atggaaactgacacgttgttgctgtgggtattgctcttgtgggtcccaggatctacgggcgac

SEQ ID NO: 39 - DNA, 66 bp, Homo sapiens
  source 1..66: mol_type = other DNA
atggatatgagggttcccgcccagcttttggggctgcttttgttgtggcttcgaggggctcggtgt

SEQ ID NO: 40 - DNA, 48 bp, synthetic construct
  source 1..48: mol_type = other DNA
atgaagtgtctgttgtacctggcgtttctgttcattggtgtaaactgt

SEQ ID NO: 41 - DNA, 84 bp, Homo sapiens
  source 1..84: mol_type = other DNA
atgaatatcaaaggaagtccgtggaagggtagtctcctgctgctcctcgtatctaaccttctcctttgtcaatccgtggcaccc

SEQ ID NO: 42 - DNA, 54 bp, Homo sapiens
  source 1..54: mol_type = other DNA
atgaaatgggtaacattcatatcacttctctttctgttcagctctgcgtattct

SEQ ID NO: 43 - DNA, 57 bp, synthetic construct
  source 1..57: mol_type = other DNA
atgacaaggcttactgttttggctctcctcgctggactcttggcttcctcccgagca

SEQ ID NO: 44 - DNA, 51 bp, Homo sapiens
  source 1..51: mol_type = other DNA
atgagggcttggattttttttctgctctgccttgccggtcgagccctggcg

SEQ ID NO: 45 - DNA, 48 bp, Homo sapiens
  source 1..48: mol_type = other DNA
atgcctcttctgcttttgcttcctcttttgtgggcaggtgccctcgca

SEQ ID NO: 46 - DNA, 87 bp, Homo sapiens
  source 1..87: mol_type = other DNA
atgaactctttctcaacctctgcgtttggtccggtcgctttctcccttgggctcctgcttgtcttgccagcagcgtttcctgcgcca

SEQ ID NO: 47 - DNA, 60 bp, Homo sapiens
  source 1..60: mol_type = other DNA
atgacaagtaaactggcggtagccttgctcgcggcctttttgatttccgcagccctttgt

SEQ ID NO: 48 - DNA, 69 bp, Homo sapiens
  source 1..69: mol_type = other DNA
atgaaggtaagtgcagcgttgctttgccttctcctcattgcagcgacctttattcctcaagggctggcc

SEQ ID NO: 49 - DNA, 78 bp, Homo sapiens
  source 1..78: mol_type = other DNA
atgggagcggcagctagaacacttcgacttgcccttgggctcttgctccttgcaaccctccttagacctgccgacgca

SEQ ID NO: 50 - DNA, 63 bp, Homo sapiens
  source 1..63: mol_type = other DNA
atgtcaccgttgttgcggagattgctgttggccgcacttttgcaactggctcctgctcaagcc

SEQ ID NO: 51 - DNA, 63 bp, Homo sapiens
  source 1..63: mol_type = other DNA
atgaataacctgctctgttgtgcgctcgtgttcctggacatttctataaaatggacaacgcaa

SEQ ID NO: 52 - DNA, 69 bp, Homo sapiens
  source 1..69: mol_type = other DNA
atgcaaatgtctcctgcccttacctgtctcgtacttggtcttgcgctcgtatttggagagggatcagcc

SEQ ID NO: 53 - DNA, 102 bp, Homo sapiens
  source 1..102: mol_type = other DNA
atggcaagggctgcactcagtgctgccccgtctaatcccagattgcttcgagttgcattgcttcttctgttgctggttgcagctggtaggagagcagcgggt

SEQ ID NO: 54 - DNA, 63 bp, Homo sapiens
  source 1..63: mol_type = other DNA
atgaatgcaaaagtcgtggtcgtgctggttttggttctgacggcgttgtgtcttagtgatggg

SEQ ID NO: 55 - DNA, 66 bp, Homo sapiens
  source 1..66: mol_type = other DNA
atggaacgcattgtgatctgcctgatggtcatcttcctgggcaccttagtgcacaagtcgagcagc

SEQ ID NO: 56 - protein, 22 aa, Homo sapiens
MCHQQLVISWFSLVFLASPLVA

SEQ ID NO: 57 - protein, 22 aa, synthetic construct
MCHQQLVISWFSLVFLASPLVA

SEQ ID NO: 58 - protein, 14 aa, synthetic construct
MQLLSCIALILALV

SEQ ID NO: 59 - protein, 20 aa, Homo sapiens
MYRMQLLSCIALSLALVTNS

SEQ ID NO: 60 - protein, 15 aa, Homo sapiens
MNLLLILTFVAAAVA

SEQ ID NO: 61 - protein, 17 aa, Gaussia sp.
MGVKVLFALICIAVAEA

SEQ ID NO: 62 - protein, 24 aa, Homo sapiens
MPMGSLQPLATLYLLGMLVASCLG

SEQ ID NO: 63 - protein, 21 aa, Mus musculus
METDTLLLWVLLLWVPGSTGD

SEQ ID NO: 64 - protein, 22 aa, Homo sapiens
MDMRVPAQLLGLLLLWLRGARC

SEQ ID NO: 65 - protein, 16 aa, synthetic construct
MKCLLYLAFLFIGVNC

SEQ ID NO: 66 - protein, 28 aa, Homo sapiens
MNIKGSPWKGSLLLLLVSNLLLCQSVAP

SEQ ID NO: 67 - protein, 18 aa, Homo sapiens
MKWVTFISLLFLFSSAYS

SEQ ID NO: 68 - protein, 19 aa, synthetic construct
MTRLTVLALLAGLLASSRA

SEQ ID NO: 69 - protein, 17 aa, Homo sapiens
MRAWIFFLLCLAGRALA

SEQ ID NO: 70 - protein, 16 aa, Homo sapiens
MPLLLLLPLLWAGALA

SEQ ID NO: 71 - protein, 29 aa, Homo sapiens
MNSFSTSAFGPVAFSLGLLLVLPAAFPAP

SEQ ID NO: 72 - protein, 20 aa, Homo sapiens
MTSKLAVALLAAFLISAALC

SEQ ID NO: 73 - protein, 23 aa, Homo sapiens
MKVSAALLCLLLIAATFIPQGLA

SEQ ID NO: 74 - protein, 26 aa, Homo sapiens
MGAAARTLRLALGLLLLATLLRPADA

SEQ ID NO: 75 - protein, 21 aa, Homo sapiens
MSPLLRRLLLAALLQLAPAQA

SEQ ID NO: 76 - protein, 21 aa, Homo sapiens
MNNLLCCALVFLDISIKWTTQ

SEQ ID NO: 77 - protein, 23 aa, Homo sapiens
MQMSPALTCLVLGLALVFGEGSA

SEQ ID NO: 78 - protein, 34 aa, Homo sapiens
MARAALSAAPSNPRLLRVALLLLLLVAAGRRAAG

SEQ ID NO: 79 - protein, 21 aa, Homo sapiens
MNAKVVVVLVLVLTALCLSDG

SEQ ID NO: 80 - protein, 22 aa, Homo sapiens
MERIVICLMVIFLGTLVHKSSS

SEQ ID NO: 81 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcactgtaatatcatccgctctttactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 82 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttgatcggccaatatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 83 - protein, 21 aa, Homo sapiens
MALPVTALLLPLALLLHAARP

SEQ ID NO: 84 - DNA, 63 bp, Homo sapiens
  source 1..63: mol_type = other DNA
atggctctccctgtaactgccctgcttcttccccttgccttgcttctccatgccgctagaccc

SEQ ID NO: 85 - protein, 22 aa, Homo sapiens
MLLLVTSLLLCELPHPAFLLIP

SEQ ID NO: 86 - DNA, 66 bp, Homo sapiens
  source 1..66: mol_type = other DNA
atgctgctgctggtcacatctctgctgctgtgcgagctgccccatcctgcctttctgctgatccct

SEQ ID NO: 87 - DNA, 66 bp, Homo sapiens
  source 1..66: mol_type = other DNA
atgctgctgctggttacatctctgctgctgtgcgagctgccccatcctgcctttctgctgatccct

SEQ ID NO: 88 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagtttagaacttcgtatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 89 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgctaacattaagtagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 90 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttatagataacgccgtcattgtaagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 91 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatactttctctaacggacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 92 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccactaacatcgttctcagctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 93-95 - skipped (3 entries)

SEQ ID NO: 96 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacctatacagtgttcagcgtgttacttgtgaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 97 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttcgtacaagtatcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 98-99 - skipped (2 entries)

SEQ ID NO: 100 - protein, 42 aa, synthetic construct
AAAIEVMYPPPYLDNEKSNGTIIHVKGKHLCPSPLFPGPSKP

SEQ ID NO: 101 - protein, 12 aa, synthetic construct
ESKYGPPCPSCP

SEQ ID NO: 102 - protein, 12 aa, synthetic construct
ESKYGPPAPSAP

SEQ ID NO: 103 - protein, 12 aa, synthetic construct
ESKYGPPCPPCP

SEQ ID NO: 104 - protein, 12 aa, synthetic construct
EPKSCDKTHTCP

SEQ ID NO: 105 - protein, 86 aa, synthetic construct
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 106 - protein, 45 aa, synthetic construct
TTTPAPRPPTPAPTIALQPLSLRPEACRPAAGGAVHTRGLDFACD

SEQ ID NO: 107 - protein, 132 aa, synthetic construct
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 108 - protein, 34 aa, synthetic construct
ACPTGLYTHSGECCKACNLGEGVAQPCGANQTVC

SEQ ID NO: 109 - protein, 20 aa, synthetic construct
AVGQDTQEVIVVPHSLPFKV

SEQ ID NO: 110 - DNA, 126 bp, synthetic construct
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 111 - DNA, 36 bp, synthetic construct
  source 1..36: mol_type = other DNA
gaaagcaagtacggtccaccttgccctagctgtccg

SEQ ID NO: 112 - DNA, 36 bp, synthetic construct
  source 1..36: mol_type = other DNA
gaatccaagtacggccccccagcgcctagtgcccca

SEQ ID NO: 113 - DNA, 36 bp, synthetic construct
  source 1..36: mol_type = other DNA
gaatctaaatatggcccgccatgcccgccttgccca

SEQ ID NO: 114 - DNA, 36 bp, synthetic construct
  source 1..36: mol_type = other DNA
gaaccgaagtcttgtgataaaactcatacgtgcccg

SEQ ID NO: 115 - DNA, 258 bp, synthetic construct
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 116 - DNA, 396 bp, synthetic construct
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 117 - DNA, 102 bp, synthetic construct
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 118 - DNA, 60 bp, synthetic construct
  source 1..60: mol_type = other DNA
gctgtgggccaggacacgcaggaggtcatcgtggtgccacactccttgccctttaaggtg

SEQ ID NO: 119 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatcagtctctgaataagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 120 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgccctatataatacccgctagcatacaaatagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 121 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcgttgctcatatacacagcaaagctggacaaacacagcaatccaggcaggggatttccaactcaactctggtatataagctgcatgcaaagtcctttttctgtctctggtttctggccccttgtctgcagag

SEQ ID NO: 122 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaacgtctgttagtagtactcaactctggtatataagctgcatgcaaagtcctttttctgtctctggtttctggccccttgtctgcagag

SEQ ID NO: 123 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaggtgaattttctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgcgcgtagaacttcgtaacattaagctattccaagccagtctccagataacgccgtcattgtatcaggtgttggacataggaaaacagttaacatcgttctcagctagtttgctgagccccagaggcctgagatgtgggatgggggtcctgggcctcctcccaaggtacctggcctttcagctctgcttgccccaagggaaggactggttcccgacgtctgttagtagtattacccgtgtatttcggtcttcgagcaattactttattgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 124 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaggtgaattttctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttgagtaaactaactttcaaccgctctttctcttctgacctttctcataaggatgggaagtcacttgtcccctcccccatcggccgattccttgaaattttaagatttgtctgaatatgagctggccagcccctctccttagaaatcccctcttggttcccaggactgcgcgtagaacttcgtaacattaagctattccaagccagtctccagataacgccgtcattgtatcaggtgttggacataggaaaacagttaacatcgttctcagctagtttgctgagccccagaggcctgagatgtgggatgatatacagtgttcagcgtgttacgtacctggcctttcagctctgcttgccccaagggaaggactggttcccgacgtctgttagtagtattacccgtgtatttcggtcttcgagcaattactttattgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcgcagaatatcgatatcttcacacagatcctgcacacacccagacagctggcgctcacacattcaccgttggcctgcctctgttcaccctcc

SEQ ID NO: 125 - DNA, 1875 bp, synthetic construct
  source 1..1875: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagataacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactggctattccaagccagtctcccaggtgttggacataggaaaacagtgtttgctgagccccagaggcctgagatgtgggatgggggtcctgggcctcctcccaaggtacctggcctttcagctctgcttgccccaagggaaggactggttcccttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 126 - DNA, 1808 bp, synthetic construct
  source 1..1808: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagataacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgttttctcttctgacctttctcataaggatgggaagtcacttgtcccctcccccatcggccgattccttgaaattttaagatttgtctgaatatgagctggccagcccctctccttagaaatcccctcttggttcccaggactggctattccaagccagtctcccaggtgttggacataggaaaacagtgtttgctgagccccagaggcctgagatgtgggatggtacctggcctttcagctctgcttgccccaagggaaggactggttcccttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcattcacacagatcctgcacacacccagacagctggcgctcacacattcaccgttggcctgcctctgttcaccctcc

SEQ ID NO: 127-131 - skipped (5 entries)

SEQ ID NO: 132 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 133 - DNA, 2000 bp, synthetic construct
  source 1..2000: mol_type = other DNA
caattgaacctccacatttaaaactttgcgcttcttttgccagagattcaggttttaaaaaacaaaaagcctttgtgcttcaaaggacgctatcaagaaagtgaaaagatgatccatagtaagggagaaaatatttacagatatgtatcagataagggattgtaaccagaatatataaagaagtcttacaactcaataataaaaaataaataacccaattttttaaatgggcaaaggatttgaatagaaatttctcaaaaaataatacaaatggtcaataagagcatgaaaagatgctcagcataggtagtcattaggaaaacgcatatcaaaactacaaagaggccgggcatggtggctcatgcctgcaatcccagcactttgggaggccgaggctggcagatcaccttaggttaggagtttgagaccagcttggccaacatggtgaaaccccgtttctactaaaaatacaaaaattagccaggcatggtggcacgtgcctgtagtcccagctacttgggaggctgaggcaggagaatcacttgaacccaggaggtggagactgcagtaagccaagatcgtgccactgcactccagcctgtgcaacagagtaagactccatctccaaaaaaaaaaaagaaaaaagaaaaaaaacacaatgagataccactttacagccactaggatggctagaatcaaaagaatggacaatagtaagttttgttgacaatgtggaaccctcacgcattgttgatgggaacaaaatggtgtagtcactttggaaaacagtttgatagttcgtcaaaatgttaaatatataattaccatatgactcaacaatttcacttctaggtatatacccaagagaaataaagcatatgtccacacaaacacttgcacataaatatttatagcagcatcattcataatagctaaaagggaaaacagcccaaacatccaactgatgacagacaaacaaaatgtggcatatccatacaatggaatgttattcagccataaaaaggaatggggaatggatccataccaccacataggtaaccttgaaaacactaactgaaagaagccagatgtgaaggtcacatattgatacttctatttatgtgaaatgttcagaataggaaaatctatagagccagaaagtagatttgtggttgccaggggctttcgatgacaggaggggaggggagcagtcattgctagtgattacagggttcttgagagggagtgagaaaaaatgttctaaaatgtattgtggtgatggttgcataactctgtgaacatacaaaaaccattgaattctatactttaaaaaggtaaattttatgggatgtggattatagctcaataaagctgtcattaagaaaggaaaaggaagggaggggagaagagggaaaggggagggaggggaggggaggggaaggggggagaaaaaagatacatcgtggcatatctcttcccttttacctgtcattatgtgtcagtgcctcccattggcctaaactacccagaagccagagggaaagccatttgggactatattccactgtgacactgagcagagcaagggaaggatggggacacttctctcacacatctctgactccccaacatctaacccatggcctggcatgtgatattcatgctataaatatttgttgaatcagtaaatggcccctcatctagattctgccaggcagcgcagggggctttttgaatgtaaatgacctaatgattcaggatcctatgaccagcgactgtcaccctctgtcacctttcttctgcccctttatctctgaaggcattgaaggggccctgcatgagtcagggagggctggggggaggagcaagggtgggaggggcagggaagagggtggcttctccgacaacttaaaaggggcttgaaccacttcctccccagacaggggtagtgcgaggccgggcacagccttcctgtgtggttttaccgcccagagagcgtc

SEQ ID NO: 134 - DNA, 2000 bp, synthetic construct
  source 1..2000: mol_type = other DNA
ttaaagaatttaaatgcaggattatatctaaataaatatataatttcaagataaaatgcaaatgataattctcaaaaaaagaagttgaataagataaaataataatctaaaagtaataaactggctataaattccccgattataccaacttttattggtaaaactaggaaataggagaaaattcttgaaagaaagttacagtgattttctcatgttacaaagacaggagctaaaaaaatattattgatggttgatgtaaataattagaaaaattcaggtaaaaatctttttaaagaattttaagaattaatatcaaaatgtaaaacaatgtattcctttcaaattacttaaggaaggccaagcaccgtggctcatcccagtaatcctagcactttgggagaccaaggcaggaggattgcttgaggcctggagttcatggctgcagtgagctatgattgtgccactcgactccagaatggtgacagagtgagacccatctcattaaaaaaaacaaaaatggcttcaaaaagttaaataaaatatatttaaagttagaaagaaatcaaagaaaacataaagaatcagcaaggaagcattttaaaaattacagattaccagttatacagtaaatatattttattaagcttacccattagaaagctcctttgatatgattttcttaaacatctacaagtgacacacttaaaacaaagatttaggaagcttaagaggaaaagataaagacaaaaaaataggaattacatcaaagtagaatctcagaggtaaaactatcacatgagcaaagaggatcacttttttttatgatccctaatacaactcatacaacttcatgtactaagtaacagtgctaaaatatatataaagtagctttccaccatggccaccattggagggcagcagccatgacactgcactaccctatagctgtgggcctcaacaagggccacaaggtgaccaagaatgtgagcaagcccaggcacagccactgtgggcgcctgaccaaactcactaaatttgtgtgggacatgatcccagaggtgtgtggcttcacccctaacgagtggcgtggcatggagttactgaaggtctccaaggtcaaacaggccctcaaattcatcaagaaaagggtagggacaaacatctgcaccaagagaaggcaggaggagctgagcaacgtcctggctgccatgaggaaagcagctgccaagaaggactgagccccctgccatctgcctataatgaaagctttgcagaataaaataaatataaaataaagtaataaaattaaattaaatttaaaaataaaataaagcaaaacaaaataaaatatataaagtaaaaattgttgaaaatgcaaaacaatatggacataaatacagaaacacagggaaacttctttaggcactcatttacaggtaaaaatatgaaattgaataaaggtcatctggtgtcaaataatataggccttatctattataagagtttggactgaaaagcaaaagtgagataataaaaaaaagcttttcagaatattattttgtatagatatgtgaaggatgaagggtgggtgaaaggaccaaaaacagaaacacagtcttcctgaatgaatgacaatcagaattccgctgcccaaagtagtccgacaattaaatggatttctaggaaaagctaccttaagaaggctggttaccatctgggttttcacagtgctttcacattcttatcactttcaacactactgcaaataggaagggacagtaacatttagaagagaacaaaacagaaactcttggaagcaggaaaggtgcatgactcaaagagggaaattcctgtgccataaaaggattgctggtgtataaaatgctctatatatgccaattatcaatttcctttcatgttcagcatttctactccttccaagaagagcagcaaagctgaagtagcagcagcagcaccagcagcaacagcaaaaaacaaac

SEQ ID NO: 135 - DNA, 2000 bp, synthetic construct
  source 1..2000: mol_type = other DNA
atggctgaaaacagatgttttctttcagaaatccttccactaccacttctgagcattttctcttagtatttttttttgggcattttccttctttgcactttttaaaatctcctgcagctcgctcactctctttcataagccagcagttatactgatgttaataaatagttgtttatttacttttgcttttttagaaaacaagctgaaagaattgagtgaaatgccagagagatacagatagccagttgtagggctggggttggaaatcaagtctaatttaaggcaagggcaaaatgtgcattttgtgtgtgtattttgaaccattcttgcattgtttttatttgcaaaggttatagggctcaagcagttagggagaatgcatcccagaacactaagttatgaataacagagcaacagttatgtttgtggcttgaacacattaaatctgcaataatgttttagaaatttggctttactaaaagcattaaagatgctatccagatgacattggggacaacatgtcaaatactattattaatgtagatatacaatgctattgtcattcataagagagtctgaacactcagacacattcattccctcttccttagtgacatcatcccaaatcataagatcttgtaagctctcaatatctgcttatagatttggcttatgtatcttttgtttttctagctaattgttgtagatcatcacttcaaggtgcccatatctttctagtggaaaaattattctggcctccgctgcatacaaatcaggcaaccagaattctacatatataaggtactaatactacattgaaaatagattagaatgagaagagacctgccatttgagtctctaaatttgttgcaaaattgcctctttctgggtcaggcttgcactaggcatttcctgactcccatgtcctagtgctacaccatttgcaaatggatatcctattttctgctgccaagcagtttctgttttaatttatgtcaacataatgatttccagggcattagctatgaggaagcagggagaatgggtcttcataattgtaaaatgacagtttttgaaatttgctagtaaagagatggagaatccaaaccttttcattgtaagattttagtgaaagaactcgtcaagcggatagtgaagttttagcatccttagtaaataaccacctttaaaatggaagaaaactatcctttatgataaggcaaagtagaaaagcatatttttatggcatttgctttttctcatatagctcaaacattttttttaaccagagactctttaggttatagggattgatgctcagaagttagaaaaaaataggttaagtattattaaaggaagttgttttactcattcagtcaatcataaaataatcatgctttgaagttttggtttaataaagcacattagtaactatgtaaaaaatttcaagtatatcactaagaagcaaacagctatgatttgttagataatgctgcataatgacttggtagtatttaatgtaatgcagtttgactggacatagatctaactgaaaaaaatgaagttgacacaaagtaaaaggaacagtgtaatatattaataaagaacatcataaagaattaatataggaacccagggaggtaaaaactaaaaaacataactaagccaaaaaccaatggaataagagttagattcaaactagtaggttagacgcaggaaatggattttcgtatgcctatcaaatatttaagaaccagtagaatggcctatagtagacatataaagggcctaggacttacttttgttttaaaataaagtctctctattttacaggactctttttattttaaaactttttttttccagaaacttaggcagtgctggggagtagtatgtccccaggttcagtaagcaaaatgacttcctttgcttattaaacaaacaccagaaattccagcctatcccatctgttcccttgcaggcaaagtaacatcctagac

SEQ ID NO: 136 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaattctcctgcctcagcctcccgagtagctgggattacaggcacatgccaccatgcccagctaatttttgtattttcaatagagactgggtttcaccatgttgcccaggctggtcttcaactcctgacctcaagtgatccgccctcctcagcttcccaaagtgctgggattacaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatattttatttgtagtgttgttttctatatagatatgataggtcttcatgattttttgtttgttttccttgaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggaaaaataaggaagtggagagttactatgtttctaatttttcatgtgcttctatttttttcctacttcagagccattgactaatagttgagtataacacaggttgtgtttccgggctgctgaaacatgacactaatattttcaaagaactgtggaagcctaaaaggaagccaatgagaaataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 137 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaactttttttccttataaattacccagcctcaagtattcctttatagtatgcaaaagagactaaaacagcaaatgaaaagagatctgtctctgaaagaacttattgtgacatagagagagacagaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcacttttctccctgccttttcccactgaaataacataataatataggaagaatacaaggactatagaaatacagattagtgtttgaacccttgcttaccagctactactaatatgattgtggatgaggtagcttcttacttattaacggggatactaatagaggtggttccttacaatgattccatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 138 - DNA, 2000 bp, synthetic construct
  source 1..2000: mol_type = other DNA
caagtccacactttctctcctctctcagtgacaatccagtcccagtaggacggtgaccctgggactcgagctgtgttcagagtcacctcttttcgctttgatgtgctcactttgcctgatttgaattcactcagtcttcctctttctctggcacattcaatgctttgggttcagttcgtcttgctacccaattccctccagggctgaccatttccaaaccttcaaccctcactccctttctcccaatcccactccagcctcacctactccacagacctttccaggggacgttcagctgctgcccttctctgtcctcccatggacatacattaccttcttcagttgcagctgaggtgtcttcacattgcactgttttctctagtgtgtttgttcacgtgtcttatttttggctttctttattagctggaagggccctggtgcgcccacatccctctccagagagttctgtctggtgatttgcacacgtgaggccaggagaattgccatgtaaggtctgagtgtccacctctagctctttcttgtctcttaaagaaacaaaatacaggggccgggcacaatggctcatgcctgtaatcccagcactttgggaggccgaggccggaggatagcttgagctcaggcattcgagaccagcctgggcaacatggtgaaacctcatctctactaaaaacacaaaaactaaccaggtgtggtagcacgtgcctgtaatcccagctacttgggaggctgggacacgagaatcacttgcacctgggaggtggaggtcgcagtgagccgaaatcatagcactgcacttcagcctgggtgacagaacaagaccctgtctcaaaaatataaaattaaatttaaaaataataaaataaaataaaataaaataaacaaaatacagggatgggggtgtgtgttgatccatgtggcacctcttagtgagactgcaacaatcccttgatccagaaataatccctgcttcttgcccctacatcacaatttacaatgtggcacaattattctgtagcatttgcatgttaaatgggcaagaaaacttaaaaaagaattcattgaatgtctctggttagaaaacaaagactcacctcctttgtcagagaagtgacctttagaaaccaggcttgcagatgatacaagataaactaagataaacttaacacctgcagtttacactgacattcacatttgctagccttagggactctctccaatgttgtgaggcttttcacatcacttgaaaaggagcaagaaagacctagaattcttgagtttcattaggaacctccagggatggagggggctgagtatttctatcagcctggtggcttggagccctgagctagtggccctcaaatattttaaggtcgatttgtgtctgtcctgaacctcattcacatgcaaattctttttaaatagaaatttgtaaatggcgtgcacacctctaagagcgctgcagctacttgtccagtgtcttgccttctttctgagttactgttggagccacggccaaaattagagaggcgttttctgtaatgctgagaaaagcaggaagcggtgagacgcttgtagcagaacacgcaaaactgtttggcggtggccccccctccacctcccacccaccgctctgtaatttccccaggcaaggctgaaggagcctttcttggaaaacccctgataacgttgctacacaaaggttgggagagtccctgacggcctgtgagggaccatgtggggctgggggtggataggggagcttgactctgcagtccccagggcagtctgagtccccagtgcctggcgtgggtgagctcagggctgctccgtatgggggcatccaggcctggctgctgatgccctgtggctgaggacacctccatctgcatgagccagtttggaagtccagcttgccccccaggcagtggaggccagtaagggcttctccccagattgaccactcagatcaaattgatcca

SEQ ID NO: 139 - DNA, 2000 bp, synthetic construct
  source 1..2000: mol_type = other DNA
ggtcatcaaagagtttgactgtagcattttaaaatagcgaagaaaaaaaggtaacaacactaggggtttgttaaacaatttatgatataattagagaatggaatattatgcatcaattacaaataatgtttataaagatacttaaatgacatgggaaaatgttcaagatataacaaataatagtacatatctgatgaaaactataagtaaaacatgatattaaacatgtaaaaatcttaacagaccaacaggtggaaggcagtggaccagaatattagcagtgtttatattagggtggtaagaatgtggatgaatcttgttttaatgcttataatttttctaaacattctcaatgaaatatgccttacttttatagtcagaataaaaagcaatacaagttatcttttaaaacaacccacatacacaaaacccccaactagagtaattgcacaaagcttagattatcaaaaagaagaaaaaaattactggaaaagttttgaagaagtcattgagtacaagatactgacgtaggtgccggaagaagagagcagttggccgtgctggtggaataaccctctctgcaaagggcctgggagttgaagaagggtgcagtcgggtggtggttatgaatctcagaaatcctgccacggagcctccttttgtgccctattacttaaccttgagggacatagagagcatgagacaccaaggggcttttggttgccttactgtcccactaagaacatagaatgttgttttgactttccctttgcttagggaacctcccccagatactcagctggctggctgcttgcacgtagaatgggttttgcaaagttcctagaagtgagttggaggaggcttgacataaatcaagcactgtgtgctaaatgctccagagggctaccttatgtcctacacaaatgttacatttctaatatttgtaactcctttaaacgtttatgcagatgtttcccaattcctcagtagaattcttaccattatctttttgacacaacctgtccccatcctatgaaaatctctcctctgctagagactttattctttctttctctctctcccccttccttccttccctccctccctctttctttctttccatctttctttctttctttctttctttctttctttctttctttctttctttctttctttctctttctttctttcttttctttctttctttctttctctttctctctctttctttcttcctttcttttttctctctccccttccttccttctttccttcttttcttttcttttcttttcttttctttctctctttctttctgtctttcttttctcattctgttgccctggctggagtgcagtggcatgatctcggctcatagcagcctccacctcctgggttcaagcgattctcctgccttagcctccctagtagctgggattacaggtacccaccatgatgcctggctaattttttgtattttcaatggagacggggtttcaccatgttggccaggctcgtcttgacctcctggcctcaaatgatccacccactttggcctcccaaattgctggcattacaggcgtgagccactgcacccggcctgttccttcttaagaacactttgtctcccctttaatctctgctggatttcaagcaccccttttacacaactcttgatatccatcaataaagaataattcccataagcccatcatgtagtgaccgactatttttcagtgacaaaaaaaaagtctttaaaaatagaagtaaaagtctaaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaag

SEQ ID NO: 140 - DNA, 2000 bp, synthetic construct
  source 1..2000: mol_type = other DNA
gcactgcactccagcctaggcaacgtgcgagaacctgtctcaaaaaacaaacaaaatacacacacacacacacacacacacacacacacacacacacacacaccctgcaggtgattacgatctcaagttctagaggcttacgttaaaatttacacatttgcttctactatttacaagtttatgtttcagcctgagctcttcttgtaagacagaccacgaggtaccccagatggaggaatgtgcaaagacgccggggctggagctggaatccgtcccgagacttctcaccttgacctcgaggaaggcaggaggtcggagacagggggtgggcaggctggggaaagaggaaacccgcaggacaagagggttgtggctcgtcacgtggccacttgagggcagcaagtagctgcagctggaggtagctgcctatctgattctgtttgagagtccgccaagttgggatttaaattagacatcaagaagaatttacagaccggccttgccttgttccacggtaaggggacattgggtttagggctggaggaggtagtctctgggactctgggcccggcggaaggaggaaccggcgttctaagagattagagagcctttagttgtttcctttcttaggtgtttttttttcttttcttttcttttttttttttttaacctcagtgccacggaatccgtctcttccagcccacagtgtaccttctcctgtccccgcccatatccctcctcctggaaaatttctccaatggcgctcagcgttcgcacccttcctctccgcggttttctagtccctaggccccagcccactagaagccaggatgccccctcaccccgtctcccactctccagcgccccagctccttgccgtcacaagaacgaagtaggtcaggggagggaagaggcatcttcgtgtcctgacctgattccttgccgtctattaaatatcctacattgcgggtatttttattctgtcctggaagaaaaaataaaaagattatctatccccagagcctgtcagaaggtggtgacaggtggggtgagccatttcctcgtctggggctggctctgaagattgattgggtctggccctgagtaggcctggaggccccagagaggcaggaggcctgggttacctactcgtggggaggggtccagactgttgcagagtaaggggtagccaggccccctccaccctcacgtctgctaggcttggggggcaaatcggtgctggaaatggggggtggagggataaggtggagtcatggaaggggggtgggattcaggcccctcttggggagaaaagctgggggtctttgctgcagggggagatttctgggggtgggggctggaggacaaagcatcagagacgcacagaattccgaagaacagctcaggaagtcgcagagcttggaggagctgggggcggggaacagtcaggggcccgtgacccagggatggggcgcctgtcccttccaggggtccagtcctcgggccttctctctgcagctttcccggcggaacggaggagggaaagggagccaggagctcagtgaggagcgcggggcggctgatgcttctaggggttctctcagcccgaggggctcccgccctgtcccctactctgcaccccttgccttgggagcggctgcagagctctaggggagcgctccgttagggctgagggcaggcgccgggagagcccccaacgacgccgcgccccccgccccgccccctgccccagctcccccccacccccaccgacccagcggcccccccagctccccgcctgctcccggaggccgcagcctccagctccgctcgcgctctcgccgctcctgccggctcgcccggccccgcgctccgccgtctcctcgccgcccgcccctccgccagccccggggaccgcgcggccgcagcctgagccagggccccctccctcgtcaggaccggggcagcaagcaggccgggggcaggtccgggcacccacc

SEQ ID NO: 141 - DNA, 2000 bp, synthetic construct
  source 1..2000: mol_type = other DNA
acactccgtgctgggagcaccactactctcttcaaagctgtcagacagggacgtgtaagtctgcagaagttgctgctgccttttgttcagctgtgccctacccccagaggtggagtctacagaggcaggcaggcttccttgagctgcagtgggctccaccaggttcgagcttcccagccgttgtgcttacctactcaagcctcagcagtggctgatgcccctcccccagccttgctgccgccttgcagtttgatctcagactgctgtccttgcagtgagcaagacttcgtgggtgtgggaccctccgagccaggcacgggatatagtctcctggtgtgctgtttgctaaggccattggaaaagcgcagtgttagggtgagagtgtcctgattttccaggtaccatctgtcacggcttcccttggctaggaaagggaattccctgaccctttgcacttcccggatgaggcgatgccccaccctgctctgtgggctgcactcactgtgtgacaagccccattgagatgaacctggtacctcagttggcaatgcagaaatcccctgtcttctgcatcactcaggctgggagctgtagactggagctgttcctatatttggccattttggaacttccatcctgacaatgggctaatatccagaatctacaaagatcttaaacaaatttacaagaaaacatcaaacaaccccatcaaaaagtaggcaaaggatatgaacagacacttctcaaaagaagacatttgtgcagccaacagacacatgaaaaaatgctcatcatcactggtcatcagagaaatgcaaatcgaaaccacaatgagataccatctcacaccagttagaatggcaatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatatgacccagccatcccattactgggtatatacccaaaggattataaatcatgctgctataaagacacatgcacacatatgtttattttggcatgattcacaatagcaaagagttggaaccaacccaaatgtcccatcaatgatagactggattaagaaactgtggcacatatacaccatggaatactatgcagccataaaaaaggatgagttcatcacatgttctcactcataggtgcgaattaaacaatgagaacacttggacacagggtggggaacatcatacaccggggcctgtcatgggttgggggcgggggggaagggatagcattaggagatatacctaatgtaaatgatgagttaacgggtgcagcacaccaacatggcacatgtatacatatgtaacaaacctgcacgttgtgcacatttaccctagaacttaaagtataattaaaaaattaaaataaattaataaaaaaatcttaactgttggggatgaaggataagaaataattcttcatgaactacagagtgtggttaagaccagtattattagccaaaaagaaagttagattttatacattagctctcttctattaaacatgataatcagactgaaatattgaagagtttacttgaattttcttagttgtgggggataaaacaatgatgttttacattttaaaaaattagaggtattacacttttcaaatctttgtttttgtatacctgggaggcatttttttgtgtgtttgtgatgtttcttatttgcattattacagatgcattgattgtatattattttgtgaagttattttatttctatgttgcatttttcttattaaaattatacaatctttcagatgaacaggaaggacagtgtctgtaatctctcctcaccgccatctgcagtagtcgtacaaaggtgatgttttatgtgagtaagaatttctgtgtttcatttcacaggtggggagcattaagcgggaa

SEQ ID NO: 142 - DNA, 2000 bp, synthetic construct
  source 1..2000: mol_type = other DNA
gatgaattaatgaacagatacctgaataaaaggatagaaggccaggcacggtggctcacgcccttacctactacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 143 - DNA, 2000 bp, synthetic construct
  source 1..2000: mol_type = other DNA
gatgaattaatgaacagatacctgaataaaaggatagaaggccaggcacggtggctcacgcctgtaatcccaacactttaattcagacgacaaaccattctatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 144 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccattctaagtccaattcacgacactcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 145 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgccgttgaagcttgctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 146 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcgagtcgtcagactcaattattaccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 147 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccaaattggaaccacgtatctactgcattgtaactacaacagctcgaggtattagataagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 148 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaggtgaattttctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 149 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggctactcatcactagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 150 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgacctgctagttgtccaatagtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 151 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacgtgtgtcatatagaattgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 152 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcaaacagtctaagtcctcaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 153 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattatactctacggaagtagcttgtttaaaacctatagtatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 154 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaagttctactagtacaaaggtaccagtaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 155 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttgagtaaactaactttcaaccgctctttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 156 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttcttcgttaccatctttcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 157 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcaaaacaccgttttgctgtaatatcattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 158 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgcgcgtagaacttcgtaacattaagctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 159 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccagataacgccgtcattgtatcaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 160 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagttaacatcgttctcagctagtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 161 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgatatacagtgttcagcgtgttacgtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 162 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccgacgtctgttagtagtattacccgtgtatttcggtcttcgagcaattactttattgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaaggaaagcagaaggaaagggcaaccgcagggggaagaaggggggcagggtgcgattctgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 163 - DNA, 2000 bp, synthetic construct
  source 1..2000: mol_type = other DNA
gatgaattaatgaacagatacctgaataaaaggatagaaggccaggcacggtggctcacgcctgtaatcccaacactttgggaagcggaggcaagcagatcatttgaagtcaggagttcgaaaccaggagttcgaaaccagcctagccaacaaggtgaaacctcacctctactaaaaataccaaatttagccgggcatgatggtgggtgcctgtaatcccagctactcaggaggctgaggcaggagaatcccttgagcccaggaggtggaggttgctctgagccaagatcgtgccattgtactccagcctgggtgacagagtgagactctgtctcaaaaaaaaaaaaaaaaaaaggacaaaagaagaacaatggctaacagagcttattgtgcgtcagcctctaagctaagggctttactcagggttcctcacttaatcctaagaagtagatcttctaccattcccattttacaaataaggacactaagacacagaaactggagaattttcaccttgtcgcagttagtaagtggtggaatctgaatcccaagctaggcgctctgtctgcagagtctccctcctctgcaccgtgactacccgtgctgtgccatgaccaagtggcagacaggcggtgtggagtggaggtaaagactgcaaaccctagagcacacctgggtccaaatcatgctccagtacttactagctgtgtggctttgtgctctgagcctcatgttccttatgtgtaaaataagaataataatgtgaaaataagaaaaactacatcaatggtattgttttgggaaagttaagcaaggtaatagtggtaaagtccttgacatagagcctagcacatagtaaacaatcaaaaataccattactattattatgattaatgaatggaaagaagctaagtcagaaatttaagagggaaatgttccccctccctctctccatccaaaagagggtctagggtttgggtgacgacgggtgactgggcgtttcacatttagtttctctttcaaggccttctcttctgacctttctcataaggatgggaagtcacttgtcccctcccccatcggccgattccttgaaattttaagatttgtctgaatatgagctggccagcccctctccttagaaatcccctcttggttcccaggactgagaaaagggggaaaatgaagggggctattccaagccagtctccctccagctgttcctttgacccaggtgttggacataggaaaacagtaaatagaggaaggggagggtttgctgagccccagaggcctgagatgtgggatgggggtcctgggcctcctcccaaggtacctggcctttcagctctgcttgccccaagggaaggactggttcccacatagtgggcaggcacagtgatgaccttggaggccaccccaggatctcatggttgtcctgagggtgccagggagccttcttcaccaccagcccaagaaagaagcaccagtgaggacaagggataaacctaaggaagggagcacagttcctcactgtgggccccacctttgaccatcacttctgaaccccagcttcatggttgatgatgcaaatgccatcaatgggatgacagggcaatccctctctctggagaaacagtgcacagccccctttactgggccagactgcagacttaagttctcagagcaggagccaagtgcataaaaagaaattcaccacgagtacctatcttggtctcgtttgttgcactactgccagaaagaggggggcaacaggggaatcacagaatgggacatgaaggggaatttcaggcagagaaagtgaagctctggccataaataggagtcagctgcagggctcacattcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 164-169 - skipped (6 entries)

SEQ ID NO: 170 - DNA, 10 bp, synthetic construct
  source 1..10: mol_type = other DNA
tgtaatccca

SEQ ID NO: 171 - DNA, 10 bp, synthetic construct
  source 1..10: mol_type = other DNA
cttacctact

SEQ ID NO: 172 - DNA, 22 bp, synthetic construct
  source 1..22: mol_type = other DNA
gggaagcggaggcaagcagatc

SEQ ID NO: 173 - DNA, 22 bp, synthetic construct
  source 1..22: mol_type = other DNA
aattcagacgacaaaccattct

SEQ ID NO: 174 - DNA, 21 bp, synthetic construct
  source 1..21: mol_type = other DNA
gcctagccaacaaggtgaaac

SEQ ID NO: 175 - DNA, 21 bp, synthetic construct
  source 1..21: mol_type = other DNA
ttctaagtccaattcacgaca

SEQ ID NO: 176 - DNA, 10 bp, synthetic construct
  source 1..10: mol_type = other DNA
tgtaatccca

SEQ ID NO: 177 - DNA, 10 bp, synthetic construct
  source 1..10: mol_type = other DNA
gttgaagctt

SEQ ID NO: 178 - DNA, 22 bp, synthetic construct
  source 1..22: mol_type = other DNA
aggaggctgaggcaggagaatc

SEQ ID NO: 179 - DNA, 22 bp, synthetic construct
  source 1..22: mol_type = other DNA
gagtcgtcagactcaattatta

SEQ ID NO: 180 - DNA, 54 bp, synthetic construct
  source 1..54: mol_type = other DNA
gcctgggtgacagagtgagactctgtctcaaaaaaaaaaaaaaaaaaaggacaa

SEQ ID NO: 181 - DNA, 54 bp, synthetic construct
  source 1..54: mol_type = other DNA
aattggaaccacgtatctactgcattgtaactacaacagctcgaggtattagat

SEQ ID NO: 182 - DNA, 11 bp, synthetic construct
  source 1..11: mol_type = other DNA
agaacaatggc

SEQ ID NO: 183 - DNA, 11 bp, synthetic construct
  source 1..11: mol_type = other DNA
ggtgaattttc

SEQ ID NO: 184 - DNA, 12 bp, synthetic construct
  source 1..12: mol_type = other DNA
gctctgtctgca

SEQ ID NO: 185 - DNA, 12 bp, synthetic construct
  source 1..12: mol_type = other DNA
tactcatcacta

SEQ ID NO: 186 - DNA, 16 bp, synthetic construct
  source 1..16: mol_type = other DNA
aagtggcagacaggcg

SEQ ID NO: 187 - DNA, 16 bp, synthetic construct
  source 1..16: mol_type = other DNA
tgctagttgtccaata

SEQ ID NO: 188 - DNA, 17 bp, synthetic construct
  source 1..17: mol_type = other DNA
catcaatggtattgttt

SEQ ID NO: 189 - DNA, 17 bp, synthetic construct
  source 1..17: mol_type = other DNA
cgtgtgtcatatagaat

SEQ ID NO: 190 - DNA, 19 bp, synthetic construct
  source 1..19: mol_type = other DNA
catagtaaacaatcaaaaa

SEQ ID NO: 191 - DNA, 19 bp, synthetic construct
  source 1..19: mol_type = other DNA
aacagtctaagtcctcaaa

SEQ ID NO: 192 - DNA, 34 bp, synthetic construct
  source 1..34: mol_type = other DNA
tatgattaatgaatggaaagaagctaagtcagaa

SEQ ID NO: 193 - DNA, 34 bp, synthetic construct
  source 1..34: mol_type = other DNA
actctacggaagtagcttgtttaaaacctatagt

SEQ ID NO: 194 - DNA, 26 bp, synthetic construct
  source 1..26: mol_type = other DNA
atgttccccctccctctctccatcca

SEQ ID NO: 195 - DNA, 26 bp, synthetic construct
  source 1..26: mol_type = other DNA
gttctactagtacaaaggtaccagta

SEQ ID NO: 196 - DNA, 26 bp, synthetic construct
  source 1..26: mol_type = other DNA
tcacatttagtttctctttcaaggcc

SEQ ID NO: 197 - DNA, 26 bp, synthetic construct
  source 1..26: mol_type = other DNA
tgagtaaactaactttcaaccgctct

SEQ ID NO: 198 - DNA, 13 bp, synthetic construct
  source 1..13: mol_type = other DNA
cttctgacctttc

SEQ ID NO: 199 - DNA, 13 bp, synthetic construct
  source 1..13: mol_type = other DNA
tcgttaccatctt

SEQ ID NO: 200 - DNA, 23 bp, synthetic construct
  source 1..23: mol_type = other DNA
cttgtcccctcccccatcggccg

SEQ ID NO: 201 - DNA, 23 bp, synthetic construct
  source 1..23: mol_type = other DNA
aaacaccgttttgctgtaatatc

SEQ ID NO: 202 - DNA, 23 bp, synthetic construct
  source 1..23: mol_type = other DNA
agaaaagggggaaaatgaagggg

SEQ ID NO: 203 - DNA, 23 bp, synthetic construct
  source 1..23: mol_type = other DNA
cgcgtagaacttcgtaacattaa

SEQ ID NO: 204 - DNA, 20 bp, synthetic construct
  source 1..20: mol_type = other DNA
ctccagctgttcctttgacc

SEQ ID NO: 205 - DNA, 20 bp, synthetic construct
  source 1..20: mol_type = other DNA
agataacgccgtcattgtat

SEQ ID NO: 206 - DNA, 18 bp, synthetic construct
  source 1..18: mol_type = other DNA
aaatagaggaaggggagg

SEQ ID NO: 207 - DNA, 18 bp, synthetic construct
  source 1..18: mol_type = other DNA
taacatcgttctcagcta

SEQ ID NO: 208 - DNA, 23 bp, synthetic construct
  source 1..23: mol_type = other DNA
ggggtcctgggcctcctcccaag

SEQ ID NO: 209 - DNA, 23 bp, synthetic construct
  source 1..23: mol_type = other DNA
atatacagtgttcagcgtgttac

SEQ ID NO: 210 - DNA, 53 bp, synthetic construct
  source 1..53: mol_type = other DNA
acatagtgggcaggcacagtgatgaccttggaggccaccccaggatctcatgg

SEQ ID NO: 211 - DNA, 53 bp, synthetic construct
  source 1..53: mol_type = other DNA
gacgtctgttagtagtattacccgtgtatttcggtcttcgagcaattacttta

SEQ ID NO: 212 - DNA, 55 bp, synthetic construct
  source 1..55: mol_type = other DNA
ggaaagcagaaggaaagggcaaccgcagggggaagaaggggggcagggtgcgatt

SEQ ID NO: 213 - DNA, 55 bp, synthetic construct
  source 1..55: mol_type = other DNA
gtgcataaaaagaaattcaccacgagtacctatcttggtctcgtttgttgcacta

SEQ ID NO: 214 - DNA, 43 bp, synthetic construct
  source 1..43: mol_type = other DNA
cacagaatgggacatgaaggggaatttcaggcagagaaagtga

SEQ ID NO: 215 - DNA, 43 bp, synthetic construct
  source 1..43: mol_type = other DNA
aaaaactaccaaccagttatcatttctctgtgtaatatctgaa

SEQ ID NO: 216 - DNA, 18 bp, synthetic construct
  source 1..18: mol_type = other DNA
cccagtcgccctgcagcc

SEQ ID NO: 217 - DNA, 18 bp, synthetic construct
  source 1..18: mol_type = other DNA
cgcagaatatcgatatct

SEQ ID NO: 218 - DNA, 15 bp, synthetic construct
  source 1..15: mol_type = other DNA
acccagacagctggc

SEQ ID NO: 219 - DNA, 15 bp, synthetic construct
  source 1..15: mol_type = other DNA
cgaatagcacctata

SEQ ID NO: 220 - DNA, 11 bp, synthetic construct
  source 1..11: mol_type = other DNA
gttaagtggct

SEQ ID NO: 221 - DNA, 11 bp, synthetic construct
  source 1..11: mol_type = other DNA
cttacctacta

SEQ ID NO: 222 - DNA, 17 bp, synthetic construct
  source 1..17: mol_type = other DNA
attgttttttttttttt

SEQ ID NO: 223 - DNA, 17 bp, synthetic construct
  source 1..17: mol_type = other DNA
taattcgtccgatagat

SEQ ID NO: 224 - DNA, 22 bp, synthetic construct
  source 1..22: mol_type = other DNA
agtctcactctgctgcccaggc

SEQ ID NO: 225 - DNA, 22 bp, synthetic construct
  source 1..22: mol_type = other DNA
aattcagacgacaaaccattct

SEQ ID NO: 226 - DNA, 22 bp, synthetic construct
  source 1..22: mol_type = other DNA
aattctcctgcctcagcctccc

SEQ ID NO: 227 - DNA, 22 bp, synthetic construct
  source 1..22: mol_type = other DNA
ttctaagtccaattcacgacaa

SEQ ID NO: 228 - DNA, 10 bp, synthetic construct
  source 1..10: mol_type = other DNA
tgggattaca

SEQ ID NO: 229 - DNA, 10 bp, synthetic construct
  source 1..10: mol_type = other DNA
gttgaagctt

SEQ ID NO: 230 - DNA, 22 bp, synthetic construct
  source 1..22: mol_type = other DNA
ggtttcaccatgttgcccaggc

SEQ ID NO: 231 - DNA, 22 bp, synthetic construct
  source 1..22: mol_type = other DNA
agtcgtcagactcaattattac

SEQ ID NO: 232 - DNA, 37 bp, synthetic construct
  source 1..37: mol_type = other DNA
ctcctgacctcaagtgatccgccctcctcagcttccc

SEQ ID NO: 233 - DNA, 37 bp, synthetic construct
  source 1..37: mol_type = other DNA
tccctagcgatcgaagttgataaaacctaagttttgt

SEQ ID NO: 234 - DNA, 10 bp, synthetic construct
  source 1..10: mol_type = other DNA
tgggattaca

SEQ ID NO: 235 - DNA, 10 bp, synthetic construct
  source 1..10: mol_type = other DNA
gccttcataa

SEQ ID NO: 236 - DNA, 24 bp, synthetic construct
  source 1..24: mol_type = other DNA
ttttatttgtagtgttgttttcta

SEQ ID NO: 237 - DNA, 24 bp, synthetic construct
  source 1..24: mol_type = other DNA
tctcgctaataggagtaagataca

SEQ ID NO: 238 - DNA, 23 bp, synthetic construct
  source 1..23: mol_type = other DNA
tgattttttgtttgttttccttg

SEQ ID NO: 239 - DNA, 23 bp, synthetic construct
  source 1..23: mol_type = other DNA
ttctgctgcaagacctatactat

SEQ ID NO: 240 - DNA, 23 bp, synthetic construct
  source 1..23: mol_type = other DNA
attttggtttcagttttccttac

SEQ ID NO: 241 - DNA, 23 bp, synthetic construct
  source 1..23: mol_type = other DNA
ccacattgctatagtgctgtata

SEQ ID NO: 242 - DNA, 17 bp, synthetic construct
  source 1..17: mol_type = other DNA
attttatccttttctcc

SEQ ID NO: 243 - DNA, 17 bp, synthetic construct
  source 1..17: mol_type = other DNA
tgcgtaccagaatattt

SEQ ID NO: 244 - DNA, 23 bp, synthetic construct
  source 1..23: mol_type = other DNA
gttgcctcatcagccccccgcag

SEQ ID NO: 245 - DNA, 23 bp, synthetic construct
  source 1..23: mol_type = other DNA
tggtcactatcacgtatatacca

SEQ ID NO: 246 - DNA, 18 bp, synthetic construct
  source 1..18: mol_type = other DNA
aaaataaggaagtggaga

SEQ ID NO: 247 - DNA, 18 bp, synthetic construct
  source 1..18: mol_type = other DNA
cgagttcgataatacact

SEQ ID NO: 248 - DNA, 17 bp, synthetic construct
  source 1..17: mol_type = other DNA
tgcttctatttttttcc

SEQ ID NO: 249 - DNA, 17 bp, synthetic construct
  source 1..17: mol_type = other DNA
aatactggtgcttcaat

SEQ ID NO: 250 - DNA, 14 bp, synthetic construct
  source 1..14: mol_type = other DNA
tgaaacatgacact

SEQ ID NO: 251 - DNA, 14 bp, synthetic construct
  source 1..14: mol_type = other DNA
ccgatagaaagaat

SEQ ID NO: 252 - DNA, 13 bp, synthetic construct
  source 1..13: mol_type = other DNA
aagccaatgagaa

SEQ ID NO: 253 - DNA, 13 bp, synthetic construct
  source 1..13: mol_type = other DNA
tgtctgtataaag

SEQ ID NO: 254 - DNA, 20 bp, synthetic construct
  source 1..20: mol_type = other DNA
tttgtggttttattggtttt

SEQ ID NO: 255 - DNA, 20 bp, synthetic construct
  source 1..20: mol_type = other DNA
tgttaagcatactaaactgt

SEQ ID NO: 256 - DNA, 20 bp, synthetic construct
  source 1..20: mol_type = other DNA
aaactagaaaatgaaaccat

SEQ ID NO: 257 - DNA, 20 bp, synthetic construct
  source 1..20: mol_type = other DNA
tttcgagcgacgcttaatat

SEQ ID NO: 258 - DNA, 20 bp, synthetic construct
  source 1..20: mol_type = other DNA
aaagtggaagtaatttctca

SEQ ID NO: 259 - DNA, 20 bp, synthetic construct
  source 1..20: mol_type = other DNA
tagatagtacgggttccata

SEQ ID NO: 260 - DNA, 17 bp, synthetic construct
  source 1..17: mol_type = other DNA
caccagaagcagatgct

SEQ ID NO: 261 - DNA, 17 bp, synthetic construct
  source 1..17: mol_type = other DNA
cttacctactaggttaa

SEQ ID NO: 262 - DNA, 13 bp, synthetic construct
  source 1..13: mol_type = other DNA
tttttttccttat

SEQ ID NO: 263 - DNA, 13 bp, synthetic construct
  source 1..13: mol_type = other DNA
actcgaattcaga

SEQ ID NO: 264 - DNA, 20 bp, synthetic construct
  source 1..20: mol_type = other DNA
tgacatagagagagacagaa

SEQ ID NO: 265 - DNA, 20 bp, synthetic construct
  source 1..20: mol_type = other DNA
attctagccttacagcctaa

SEQ ID NO: 266 - DNA, 62 bp, synthetic construct
  source 1..62: mol_type = other DNA
ggcagggagggaatttcccataggaagggaagagtaaagagagagagagaggtcagagtcca

SEQ ID NO: 267 - DNA, 62 bp, synthetic construct
  source 1..62: mol_type = other DNA
actctacggaagtagcttgtttaaaacctatagtctcttcggagtcgttctactagtacaaa

SEQ ID NO: 268 - DNA, 30 bp, synthetic construct
  source 1..30: mol_type = other DNA
tcttgaaagggcactatttcccagaatcca

SEQ ID NO: 269 - DNA, 30 bp, synthetic construct
  source 1..30: mol_type = other DNA
tgagtaaactaactttcaaccgctcttcgt

SEQ ID NO: 270 - DNA, 16 bp, synthetic construct
  source 1..16: mol_type = other DNA
ggtagggaatcccctg

SEQ ID NO: 271 - DNA, 16 bp, synthetic construct
  source 1..16: mol_type = other DNA
cttaaacaccgttttg

SEQ ID NO: 272 - DNA, 22 bp, synthetic construct
  source 1..22: mol_type = other DNA
cttttctccctgccttttccca

SEQ ID NO: 273 - DNA, 22 bp, synthetic construct
  source 1..22: mol_type = other DNA
ctgtaatatcatccgctcttta

SEQ ID NO: 274 - DNA, 16 bp, synthetic construct
  source 1..16: mol_type = other DNA
tgatcggccaatattt

SEQ ID NO: 275 - DNA, 11 bp, synthetic construct
  source 1..11: mol_type = other DNA
cccataaaatc

SEQ ID NO: 276 - DNA, 11 bp, synthetic construct
  source 1..11: mol_type = other DNA
tagaacttcgt

SEQ ID NO: 277 - DNA, 10 bp, synthetic construct
  source 1..10: mol_type = other DNA
tttcactttc

SEQ ID NO: 278 - DNA, 10 bp, synthetic construct
  source 1..10: mol_type = other DNA
aacattaagt

SEQ ID NO: 279 - DNA, 20 bp, synthetic construct
  source 1..20: mol_type = other DNA
aatgagatatttcacacttc

SEQ ID NO: 280 - DNA, 20 bp, synthetic construct
  source 1..20: mol_type = other DNA
tagataacgccgtcattgta

SEQ ID NO: 281 - DNA, 11 bp, synthetic construct
  source 1..11: mol_type = other DNA
aagttaattaa

SEQ ID NO: 282 - DNA, 11 bp, synthetic construct
  source 1..11: mol_type = other DNA
tttctctaacg

SEQ ID NO: 283 - DNA, 20 bp, synthetic construct
  source 1..20: mol_type = other DNA
agattcctactcttttctaa

SEQ ID NO: 284 - DNA, 20 bp, synthetic construct
  source 1..20: mol_type = other DNA
ctaacatcgttctcagctaa

SEQ ID NO: 285 - DNA, 28 bp, synthetic construct
  source 1..28: mol_type = other DNA
caggatggggaggaaaagagttagggaa

SEQ ID NO: 286 - DNA, 28 bp, synthetic construct
  source 1..28: mol_type = other DNA
tatacagtgttcagcgtgttacttgtga

SEQ ID NO: 287 - DNA, 11 bp, synthetic construct
  source 1..11: mol_type = other DNA
ggaggaaaagg

SEQ ID NO: 288 - DNA, 11 bp, synthetic construct
  source 1..11: mol_type = other DNA
cgtacaagtat

SEQ ID NO: 289 - DNA, 11 bp, synthetic construct
  source 1..11: mol_type = other DNA
tttcctgagaa

SEQ ID NO: 290 - DNA, 11 bp, synthetic construct
  source 1..11: mol_type = other DNA
agtctctgaat

SEQ ID NO: 291 - DNA, 29 bp, synthetic construct
  source 1..29: mol_type = other DNA
cccagcaaaagtgaaagaaaattcatggg

SEQ ID NO: 292 - DNA, 29 bp, synthetic construct
  source 1..29: mol_type = other DNA
ccctatataatacccgctagcatacaaat

SEQ ID NO: 293 - DNA, 12 bp, synthetic construct
  source 1..12: mol_type = other DNA
aagaacaaagag

SEQ ID NO: 294 - DNA, 12 bp, synthetic construct
  source 1..12: mol_type = other DNA
gttgctcatata

SEQ ID NO: 295 - DNA, 16 bp, synthetic construct
  source 1..16: mol_type = other DNA
ggcaggggatttccaa

SEQ ID NO: 296 - DNA, 16 bp, synthetic construct
  source 1..16: mol_type = other DNA
acgtctgttagtagta

SEQ ID NO: 297 - DNA, 91 bp, synthetic construct
  source 1..91: mol_type = other DNA
gatgagtcatcacgtccgaacacttccgccttacagctgggagcaggtgcagtcgcgtaatcacgtcaccattatccaaggggattcccct

SEQ ID NO: 298 - DNA, 91 bp, synthetic construct
  source 1..91: mol_type = other DNA
tcactttcactttcttataactgcacctgctccctacgactccggggaattcagtccacaagatgagtcatcagctggctagtcacgtgac

SEQ ID NO: 299 - DNA, 97 bp, synthetic construct
  source 1..97: mol_type = other DNA
gggaaacggaaaccgaaactgtataggggaatcccctacgttcattgcgtgggcgttggagttgtatcacgtcaccttgcgttctcacttcctgctc

SEQ ID NO: 300 - DNA, 97 bp, synthetic construct
  source 1..97: mol_type = other DNA
tcagtttcattttccaactaagattgtgcaatattccacttagtcacgtgacttggtcccacacttccgcctaagagggaaacggaaaccgaaactg

SEQ ID NO: 301 - DNA, 99 bp, synthetic construct
  source 1..99: mol_type = other DNA
gggaaacggaaaccgaaactgaagttgcgtgggcgtaggtagtctgcacctgctcccttagaagagcaggaagtgagagtcagaggtcactgcaggtca

SEQ ID NO: 302 - DNA, 94 bp, synthetic construct
  source 1..94: mol_type = other DNA
gggagcaggtgcagatgaagaacgcccacgcattgatgacttttccaggaaaatcacagtttcggtttccgtttccctaggaaaatgaaactga

SEQ ID NO: 303 - DNA, 94 bp, synthetic construct
  source 1..94: mol_type = other DNA
ggaaaatgaaactgaatcctctcacttcctgctctaggcagggagcaggtgcagtgatgctgatgactcatcatcgagtaacgaaaccgaaact

SEQ ID NO: 304 - DNA, 90 bp, synthetic construct
  source 1..90: mol_type = other DNA
aacagctgtttctcagggaaacggaaaccgaaactgtattaggcggaagtgttgcttatcactttcactttcaacgctaatcacgtcacc

SEQ ID NO: 305 - DNA, 93 bp, synthetic construct
  source 1..93: mol_type = other DNA
cacttccgccttagaggaaacgaaaccgaaactattctatatcacgtcacctatctacaaggggattcccctaagagatcagtttcattttcc

SEQ ID NO: 306 - DNA, 94 bp, synthetic construct
  source 1..94: mol_type = other DNA
aggggaatccccttgtcgaatgatgagtcatctacgtctaacgaaaccgaaactatactcaggtgacgtgattgctcaaggaaaatgaaactga

SEQ ID NO: 307 - DNA, 92 bp, synthetic construct
  source 1..92: mol_type = other DNA
gatgagtcatctggtcagaggtcactgcaggtcattactgtcacgtgacaccagtgatacgcccacgcaatacaacagtttcattatgactc

SEQ ID NO: 308 - DNA, 90 bp, synthetic construct
  source 1..90: mol_type = other DNA
gtcacgtgacacatctaggaaaatgaaactgaatgggaaacggaaaccgaaactgttagtttcggtttcgttaggttgtgaattccccgg

SEQ ID NO: 309 - DNA, 94 bp, synthetic construct
  source 1..94: mol_type = other DNA
tgacctgcagtgacctctgtcaaggggattcccctaatcagtttcggtttccgtttccctcagagtcacgtgactcgtcgaggtaacagctgtt

SEQ ID NO: 310 - DNA, 97 bp, synthetic construct
  source 1..97: mol_type = other DNA
gggaaacggaaaccgaaactgatgaaggcggaagtgtgttcgacaccggggaattcatatcaaatattgcacaatctagtctggaaaatgaaactga

SEQ ID NO: 311 - DNA, 91 bp, synthetic construct
  source 1..91: mol_type = other DNA
gaaagtgaaagtgaatttcgtccggggaattctcagacttagatgagtcatcttccatagtacgcccacgcattagtattaaacagctgtt

SEQ ID NO: 312 - DNA, 95 bp, synthetic construct
  source 1..95: mol_type = other DNA
gaattccccggacaagccttgatgactcatctagtcagtttcggtttccgtttcccaattgacctgcagtgacctctgaaagaaagtgaaagtga

SEQ ID NO: 313 - DNA, 92 bp, synthetic construct
  source 1..92: mol_type = other DNA
gacacctgtttgcggctataaacagctgttagtcgggtttaccagctgcttctcgtctgaaagtgaaagggataacatggaagagaaagtga

SEQ ID NO: 314 - DNA, 90 bp, synthetic construct
  source 1..90: mol_type = other DNA
cacttccgccttagagggaaacggaaaccgaaactgacaaggggaatccccttcatgtctgcacctgctcccaaggcgaggtgacgtgat

SEQ ID NO: 315 - DNA, 91 bp, synthetic construct
  source 1..91: mol_type = other DNA
aggggattccccttggcggcttttcctggaaatacgttagggagcaggtgcagagcgaagtttcggtttcgtttcgttataggcggaagtg

SEQ ID NO: 316 - DNA, 97 bp, synthetic construct
  source 1..97: mol_type = other DNA
atcacgtcacctatcactctcacttcctgctcaaggctagatgactcatcatttcagaggtcactgcaggtcataacagtttcggtttccgtttccc

SEQ ID NO: 317 - DNA, 92 bp, synthetic construct
  source 1..92: mol_type = other DNA
tttccaggaaaattcttaaagatgactcatctagcgattcagtttcattttcctgcgctaggtgacgtgatttaaagctaggggaatcccct

SEQ ID NO: 318 - DNA, 90 bp, synthetic construct
  source 1..90: mol_type = other DNA
aacagctgttatagcgcaatgcgtgggcgtattagggaaacggaaaccgaaactgatctccggggaattcaagtgcaagattgtgcaata

SEQ ID NO: 319 - DNA, 97 bp, synthetic construct
  source 1..97: mol_type = other DNA
ggaaaatgaaactgatgaatttaacagctgtttgtacattgaaagtgaaagtgaaaaactagaattccccggattagggaaacggaaaccgaaactg

SEQ ID NO: 320 - DNA, 94 bp, synthetic construct
  source 1..94: mol_type = other DNA
ctgcacctgctcccacctatctccacctgccgcctccgttctgcacctgccctcagcataacgaaaccgaaactttccaatcgaaagcgaaaca

SEQ ID NO: 321 - DNA, 92 bp, synthetic construct
  source 1..92: mol_type = other DNA
ctgcacctgccctcatgtcaccgcacctgccgccatcccactccacctgccgccacggcattttctaggaagagtcctcgacttccgggaag

SEQ ID NO: 322 - DNA, 94 bp, synthetic construct
  source 1..94: mol_type = other DNA
ggaaaatgaaactgaatttcagtttcattatgactcacagctgtcacgtgacagagagtttcactttcactttcattgctctgcacctgctccc

SEQ ID NO: 323 - DNA, 93 bp, synthetic construct
  source 1..93: mol_type = other DNA
tgcgtgggcgttatcggagggagcaggtgcagttgtgaaaagatcaaaggaataaacgtgatgactcatcatattgacctgcagtgacctctg

SEQ ID NO: 324 - DNA, 97 bp, synthetic construct
  source 1..97: mol_type = other DNA
gggagcaggtgcagtgtccctcacttccgccttccgatatcagtttcattttccagttctaggggaatcccctagagggaaacggaaaccgaaactg

SEQ ID NO: 325 - DNA, 96 bp, synthetic construct
  source 1..96: mol_type = other DNA
agtttcggtttcgttttcagtttcggtttccgtttcccaagttgtcacgtgacaacgctactaggcggaagtgtatcttgagtcataatgaaactg

SEQ ID NO: 326 - DNA, 94 bp, synthetic construct
  source 1..94: mol_type = other DNA
aggcggaagtgttgtatcagtttcattatgactcaccgttattgcacaatcacgacatctcacttcctgctcagttgacctgcagtgacctctg

SEQ ID NO: 327 - DNA, 89 bp, synthetic construct
  source 1..89: mol_type = other DNA
acgcccacgcaagtctcatcactttcactttctacccagagcaggaagtgagtacgtatccggggaattcagtaaacgagtcacgtgac

SEQ ID NO: 328 - DNA, 93 bp, synthetic construct
  source 1..93: mol_type = other DNA
gtcacgtgacatagtactaggggaatcccctaaagtctatgcgtgggcgtagtcgatattttcctggaaatcgtcagaggtcactgcaggtca

SEQ ID NO: 329 - DNA, 92 bp, synthetic construct
  source 1..92: mol_type = other DNA
gtcacgtgacaccaacagtttcggtttccgtttcccaataggggaatcccctagaacgctgatgagtcatcaaaatctctcacttcctgctc

SEQ ID NO: 330 - DNA, 100 bp, synthetic construct
  source 1..100: mol_type = other DNA
ccgcacctgccgccttgccactccacctgccgccaaacaaaggtcacctcaggccaactctgagggcactaaaggtcaatacagaggtcactgcaggtca

SEQ ID NO: 331 - DNA, 90 bp, synthetic construct
  source 1..90: mol_type = other DNA
gtcacgtgcctccagcagtctgagtcagcatgattagcaaagtaaacaaatatgtagagtcataatgaaactgaatctaaaagataagaa

SEQ ID NO: 332 - DNA, 91 bp, synthetic construct
  source 1..91: mol_type = other DNA
cagtttcattatgactcagaaacaatgtgcagtgtttgcagatgtcacgtgcctcctcgttagtttcggtttcggtcgcttgtaaatgact

SEQ ID NO: 333 - DNA, 92 bp, synthetic construct
  source 1..92: mol_type = other DNA
tagtttcggtttcggtacagagtcataatgaaactgagacaaaaggtgtgaaaaccattcgcatagtcatttaccctaagcaggcacgtgac

SEQ ID NO: 334 - DNA, 90 bp, synthetic construct
  source 1..90: mol_type = other DNA
aagtaaacaaattgaaagtccacttccggtctaataccttgtcacgtgcctcgtcaataaaataaacaatttacctacatatcaggttac

SEQ ID NO: 335 - DNA, 89 bp, synthetic construct
  source 1..89: mol_type = other DNA
ggcacgtgacatagcgactcaagtcatttcgtctgtgaatgcacctgttatactactccacttccggtcttatgtaaattcttatcttt

SEQ ID NO: 336 - DNA, 90 bp, synthetic construct
  source 1..90: mol_type = other DNA
ccacttccggtcttgtccgtaaacaggtgcaagtaactaagtaacctgataatacgcgtactaatgacttatgtccctgtggcacgtgac

SEQ ID NO: 337 - DNA, 89 bp, synthetic construct
  source 1..89: mol_type = other DNA
gtaacctgatatatccattatgctgactcagatctcaaacaatgtgcagtgtttatctccacttccggtctactgacaagtcacgtgcc

SEQ ID NO: 338 - DNA, 92 bp, synthetic construct
  source 1..92: mol_type = other DNA
ccgaaaccgaaactaatagcttatatttacataatcgatgacaaaatgacttcctcgaattaaataaacaatttagcatcctgtcacgtgcc

SEQ ID NO: 339 - DNA, 90 bp, synthetic construct
  source 1..90: mol_type = other DNA
gtaacctgatatccccgaatggcacgtgacttcactaccgaaaccgaaactaaacgtctttcttatctttactttatcatttgtttactt

SEQ ID NO: 340 - DNA, 89 bp, synthetic construct
  source 1..89: mol_type = other DNA
caaaggtcagactcgatcaaaataaacaatttaagttatagtcacgtgcctactttatagtttcggtttcggatgtgacagctaatgga

SEQ ID NO: 341 - DNA, 88 bp, synthetic construct
  source 1..88: mol_type = other DNA
ttttccatcagtgcgtagcatgactcagcattcgtagacaagtcatttatactaataaaataaacaattagacttgttgtcacgtgcc

SEQ ID NO: 342 - DNA, 88 bp, synthetic construct
  source 1..88: mol_type = other DNA
tgcacctgtttcattccatgctgagtcatgcaggaccctggcacgtgacattttataacactgcacattgttagtggctgctaatgga

SEQ ID NO: 343 - DNA, 90 bp, synthetic construct
  source 1..90: mol_type = other DNA
atatttacataatactagcgcttggaaaaacacgttcatagaccggaagtggtcttacagtttcattatgactcttggcacaaaggtcag

SEQ ID NO: 344 - DNA, 94 bp, synthetic construct
  source 1..94: mol_type = other DNA
tttgtttactttttaaacattgctgactcagacagtgacaggaagcagatgtgagtcgtagaccggaagtggtaactgagtcataatgaaactg

SEQ ID NO: 345 - DNA, 95 bp, synthetic construct
  source 1..95: mol_type = other DNA
cacatctgcttcctgtagagtccacttccggtctagtacgaacaaaggtcagtgtagtttagtttcggtttcggacttcagtttcattatgactc

SEQ ID NO: 346 - DNA, 94 bp, synthetic construct
  source 1..94: mol_type = other DNA
gagtcataatgaaactgaagtccgaaaccgaaactaacataagcatgactcagcatcggggtatatcaggttactcttcgcgtatatttacata

SEQ ID NO: 347 - DNA, 93 bp, synthetic construct
  source 1..93: mol_type = other DNA
cagtttcattatgactctcgctaaaggtgtgaaatcccgattttatgtaaatattaattctctgtagtcattatagcaataagttccattagc

SEQ ID NO: 348 - DNA, 88 bp, synthetic construct
  source 1..88: mol_type = other DNA
ttttccaaagccgatcagtttcattatgactcaacacaggaagcagatgtgtcatagaaaagataagaaacaacatttaacaggtgca

SEQ ID NO: 349 - DNA, 96 bp, synthetic construct
  source 1..96: mol_type = other DNA
aacactgcacattgtttaaagaaaagataagaaaccgagtcaggaagcagatgtgactaatagtaacctgatattgaaacagtttcattatgactc

SEQ ID NO: 350 - DNA, 94 bp, synthetic construct
  source 1..94: mol_type = other DNA
tttcacaccttttgggttaactgagtcagcattacgactcaaaatgacttcaccagattgcatgactcagcaaatgtgagtcataatgaaactg

SEQ ID NO: 351 - DNA, 91 bp, synthetic construct
  source 1..91: mol_type = other DNA
aacactgcacattgtttctcagtttcattatgactctggtgtgtaacctgatatggtgttcacatctgcttcctgaatcatataaatgact

SEQ ID NO: 352 - DNA, 90 bp, synthetic construct
  source 1..90: mol_type = other DNA
ccacttccggtcttttgacaaattgtttatttttttagagatgcacctgttacactagaagtaacctgataaagaacatacaaaggtcag

SEQ ID NO: 353 - DNA, 90 bp, synthetic construct
  source 1..90: mol_type = other DNA
tccattagcaggcctaataattgtttatttatagatctactgacctttgaatagcgcctaaatgacttcgttgcgatccacttccggtct

SEQ ID NO: 354 - DNA, 93 bp, synthetic construct
  source 1..93: mol_type = other DNA
agaccggaagtggacgtaacacatctgcttcctgtagttaaaataaacaattaaagtcgagtaacctgatatcgtgtaacaatgtgcagtgtt

SEQ ID NO: 355 - DNA, 93 bp, synthetic construct
  source 1..93: mol_type = other DNA
agaccggaagtggagccgtcttgcacctgttatccgtgtatatcaggttacttcagtgaactttttccataacgttaaacaatgtgcagtgtt

SEQ ID NO: 356 - DNA, 92 bp, synthetic construct
  source 1..92: mol_type = other DNA
gcatgactcagcatactgttttatcaggttacaagccgaatgtaatgactacatttaataagaccggaagtggagcttacttttcacacctt

SEQ ID NO: 357 - DNA, 89 bp, synthetic construct
  source 1..89: mol_type = other DNA
aatgactacacaactacgagctaatggaagcaacccgttgctgactcagtagtacaaaaagtaaacaaaagtcgcctttatgtaaatat

SEQ ID NO: 358 - DNA, 90 bp, synthetic construct
  source 1..90: mol_type = other DNA
tttgtttacttttcctattaattgtttatttaagtagcataacaggtgcatgccctaccgaaaccgaaactatgcatttaaagataagaa

SEQ ID NO: 359 - DNA, 89 bp, synthetic construct
  source 1..89: mol_type = other DNA
aaagataagaaacctcttaaaagtaaacaaaaagtatagttttcacacctttatattctaaataaacaattatggtacaatccattagc

SEQ ID NO: 360 - DNA, 95 bp, synthetic construct
  source 1..95: mol_type = other DNA
ccgaaaccgaaactatgctctaaaggtgtgaaaatcgacgatgctgagtcatgcactatattaagtaaacaaaacttaatcaggaagcagatgtg

SEQ ID NO: 361 - DNA, 90 bp, synthetic construct
  source 1..90: mol_type = other DNA
tttgtttacttaccaaatcagtaacctgatatacgacatacaaaggtcagtgtcactgcgtttttccaaccgtaagttactgagtcagca

SEQ ID NO: 362 - DNA, 88 bp, synthetic construct
  source 1..88: mol_type = other DNA
agtcatttaggtccttcgattggaaaaatcacaagggttgctgactcagtatagcgaaaataaacaattaattcacaaaagataagaa

SEQ ID NO: 363 - DNA, 92 bp, synthetic construct
  source 1..92: mol_type = other DNA
ctgagtcagcaatctgtgaaattgtttattttcgtacacttttttccaatcttagttccgaaaccgaaactaaagcacaggaagcagatgtg

SEQ ID NO: 364 - DNA, 93 bp, synthetic construct
  source 1..93: mol_type = other DNA
aacaatgtgcagtgttttggttgctgagtcatgcacgcattagtttcggtttcggtgagactaacaggtgcatcacgaaataattgtttattt

SEQ ID NO: 365 - DNA, 91 bp, synthetic construct
  source 1..91: mol_type = other DNA
ctgagtcagcaactatggtgaattttccaaagtacatcactgctaatggaaacctgtattctgacctttgatagtgcataaataaacaatt

SEQ ID NO: 366 - DNA, 90 bp, synthetic construct
  source 1..90: mol_type = other DNA
tatcaggttacaagactctaaaggtgtgaaaacccaagttgctgagtcatgctgtcgtaaaataaacaatttctgtagatctgacctttg

SEQ ID NO: 367 - DNA, 94 bp, synthetic construct
  source 1..94: mol_type = other DNA
aacactgcacattgtttatcctaaagataagaaagcggtcacaattttccataaacatagcgtgctaatggaatagtctatgcatgactcagca

SEQ ID NO: 368 - DNA, 86 bp, synthetic construct
  source 1..86: mol_type = other DNA
tggaaaaacgagtgctatgctgagtcatgctctaaataaacaggtgcataggagtccgaaaccgaaactaaacgtccgtagtcatt

SEQ ID NO: 369 - DNA, 92 bp, synthetic construct
  source 1..92: mol_type = other DNA
aacaatgtgcagtgtttcgaggtctgacctttgtcacaatgactagtcattatccgtctaccgaaaccgaaactaacgaaatatccattagc

SEQ ID NO: 370 - DNA, 100 bp, synthetic construct
  source 1..100: mol_type = other DNA
ctagtttggcaacaggcatcttccacccacctgggaggcagggttcaacactctgcctctgaccttttttccttcctctgccacctgcttaggcagccag

SEQ ID NO: 371 - DNA, 100 bp, synthetic construct
  source 1..100: mol_type = other DNA
agatgcacttcgcttttgttatttttttagtagagagggggtttcactggtttagccagaacagtctcgatctcctgacctcgtgatccgcccacctcgg

SEQ ID NO: 372 - DNA, 92 bp, synthetic construct
  source 1..92: mol_type = other DNA
gagtcataatgaaactgaactctctgacctttgtctctcacacatctgcttcctgaagagctggcacgtgactttatctaattcttatcttt

SEQ ID NO: 373 - DNA, 91 bp, synthetic construct
  source 1..91: mol_type = other DNA
tatcaggttacatggagtgtgtcacgtgccacgaagttaaaagataagaaagctttttatatggaaaatctcgactaaagaccggaagtgg

SEQ ID NO: 374 - DNA, 90 bp, synthetic construct
  source 1..90: mol_type = other DNA
tatgtaaatattaaaggtattatcaggttactagtatgtaaataaacaattttctccgtaggcacgtgactcgcgacaattcttatcttt

SEQ ID NO: 375 - DNA, 93 bp, synthetic construct
  source 1..93: mol_type = other DNA
tagtttcggtttcggtatcgcatatgtaaatatatccatgtatgcacctgtttgcattgctagtcacgtgcctataacatgcatgactcagca

SEQ ID NO: 376 - DNA, 88 bp, synthetic construct
  source 1..88: mol_type = other DNA
ctgacctttgagggcgaataggcacgtgacttttcgttaatatttacatatggcgacttgctgagtcatgctgatgcgtgaaatgact

SEQ ID NO: 377 - DNA, 90 bp, synthetic construct
  source 1..90: mol_type = other DNA
ccgaaaccgaaactattcagaaagctaatggatccgaattgtgtaacctgataaacggtttagtcacgtgccacgccgaacgtaatgact

SEQ ID NO: 378 - DNA, 90 bp, synthetic construct
  source 1..90: mol_type = other DNA
gcatgactcagcattatcataaacaggtgcaagccactaacaatgtgcagtgtttctgtactgagtcagcaatctccggtgtcacgtgcc

SEQ ID NO: 379 - DNA, 88 bp, synthetic construct
  source 1..88: mol_type = other DNA
tggaaaatatcccgagtatgcacctgtttctggtgtccacttccggtctttctacagtttcattatgactctttgtaaagtaaacaaa

SEQ ID NO: 380 - DNA, 96 bp, synthetic construct
  source 1..96: mol_type = other DNA
cagtttcattatgactcaacatagtttcggtttcggaggcatccacttccggtcttgttcgcattccattagcagcgtcgacaggaagcagatgtg

SEQ ID NO: 381 - DNA, 90 bp, synthetic construct
  source 1..90: mol_type = other DNA
cagtttcattatgactctaggttaacaggtgcaatcccgatcaaagtcattagcgttctcgtcaaaggtcagtttcccgagttttttcca

SEQ ID NO: 382 - DNA, 91 bp, synthetic construct
  source 1..91: mol_type = other DNA
tttgtttacttattggtccttgcacctgttttgatagataaggtgtgaaatgctttgacgattttccatgaacaggtaccacttccggtct

SEQ ID NO: 383 - DNA, 90 bp, synthetic construct
  source 1..90: mol_type = other DNA
aaggtgtgaaaatcgcagtagaccggaagtggagtccaataaagataagaaattggtacaggaagcagatgtgtagttctctgacctttg

SEQ ID NO: 384 - DNA, 91 bp, synthetic construct
  source 1..91: mol_type = other DNA
gctaatggataccgatattgctgagtcatgcatacgacaggaagcagatgtgtatctccgaaaccgaaactaacgggaccacttccggtct

SEQ ID NO: 385 - DNA, 92 bp, synthetic construct
  source 1..92: mol_type = other DNA
tagtttcggtttcggtgcgtgttgctgactcagacgcaacatctgacctttgtatctgcaatatgtaaatattcagagactaagtaaacaaa

SEQ ID NO: 386 - DNA, 86 bp, synthetic construct
  source 1..86: mol_type = other DNA
tggaaaaagctgactatatatgtaaatattcgacgtattgcacctgttaggtattcaaaataaacaattttacttaggtaatgact

SEQ ID NO: 387 - DNA, 90 bp, synthetic construct
  source 1..90: mol_type = other DNA
tatgtaaatataaaatcgttttcttatcttttggtgatcaggaagcagatgtgtagccatgattttccattgtagctcctcaaaggtcag

SEQ ID NO: 388 - DNA, 90 bp, synthetic construct
  source 1..90: mol_type = other DNA
ttcttatctttatatccctcagctaatggaagatcggaaaatatttacataactccgctaaacaggtgcaagccaacaaactgacctttg

SEQ ID NO: 389 - DNA, 92 bp, synthetic construct
  source 1..92: mol_type = other DNA
caggaagcagatgtgtcttcgatatcaggttactcgaacgcacattttccaaagctccaatagtttcggtttcggtggtccaatccattagc

SEQ ID NO: 390 - DNA, 92 bp, synthetic construct
  source 1..92: mol_type = other DNA
tttcacaccttaactctctttatcaggttacagttacccgctaatgacttgccttgagaaaacaggtgcatggcccaccgaaaccgaaacta

SEQ ID NO: 391 - DNA, 16 bp, synthetic construct
  source 1..16: mol_type = other DNA
tacaatgattccattt

SEQ ID NO: 392 - DNA, 2000 bp, synthetic construct
  source 1..2000: mol_type = other DNA
gcccagagaaggccctgacaaatggagaagatggaatatgtccccttcagttggattcttgctccgaagtattccttgagcctatactccaaagtgtggcattataactggtgcagacggagtctgtcttaaaactgaaaaggaggatccagagactggtacaaatgtcccacctcctttttttgttacttcgttattgtcaaataattgtaatgatgggacaagctaaatcatgagtcttttaaagtctcttcgatttgctttacacctggggggttgttgcttgtctctgctaattttagccatttcacaggcccatgggagcaattctacctaatgacagtgaatccaacagaagttcataactcactgatcacttagtttctctgcactcctcaccttataactgcagtcttgtggcatttttccaaaagagagccactgtgaatcaagcatagagttttatggcctgaatccaggttagattatttttttcaagaaaaagaaaaaaaaatctaacagttaccaagaagcagtcaggctttacaaatatcaccactttaaccatgaggccatattaggaaggcttttgactggcattcaggtttgggggagtctgaattttctatgccatggtctcagatttctgccccatatacctaggcactaatttagttcatatgtactatgtgtacctgaaaagttgtgtggcaatcaaattttcacaaatagaatcctgttttaaatacactaagaaagtacctactttatcctttaaacaagaggtcagcagactttttctacaaagggtcagatagtaaagattttacaccttttgtacaatacaatctctatctcatctacttagctctgccattgttgcataaaagcagctgtagatgatacacaaatgggtgaggctgtattccaatgaaacgttatttgcaaaaacaggtggtagattaaatttggtcccaaggcttacttgggaaaaaaaaagatcttttgaaaaagaaaaaataaatgaataattttttaaaaaattgttccctaggtcatagtttgccagcccctgccctaaacaaataattcttgaatgcctactgtggtgtgtaagatatgagtaaataccagggatacacagagaacaaaagagaaaaactgctattcttgtgaaacttggaagttggaggtaagctatttaaaataaacccacaataaagtacttcacatagtgcagactgtttctttaaatcaaaactcactccaaacaaccaattgattcactttgtaagtttgaatttttgtcttcagattcttttaaagtgggcccttagtcaggagcggtggctcatgcctgtagtcctagcactttgggaggctgaggcaggcagatcacttgaggtcaggagttcgagacaagcctggccaacatggcgaaaccccgtctccactgaaaacacaaaaattaggctggcatagtggcatttgcctgtagtcctagctactcaggaggctgaggcaggagaattgcttgaacctgggaggtggaaattgcagtgagccgagatcatgctattgtactccagcctgggcaacaaagcaagactctgtctcaaaaaaataaaaattaaaaaaataaagtagcctctagcctaagatagcttgagcctaggtgtgaatctactgccttactctgatgtaagcacagtaagtgtgggggctgcagggaatatccaggaggaacaataatttcagaggctctgtctcttcatgtccttgacctctgcttacagcagcaatacttttactcagacttcctgtttctggaacttgccttcttttttgctgtgtttatacttcccttgtctgtggttagataagtataaagccctagatctaagcttctctgtcttcctccctccctcccttcctcttactctcattcatttcatacacactggctcacacatctactctctctctctatctctctcaga

SEQ ID NO: 393 - DNA, 2000 bp, synthetic construct
  source 1..2000: mol_type = other DNA
aagcgatccttccaccttggcctcccaagcagctgggactacaggcacaggccaccactcctggctaacttttttctattttttgcagagatgggggtgctcactttgctgcccaggctggtcttgaatgtccatgctcaagtgatcctctcgcctccgcctgccaaagggaaggaaaaggttctggctccagttttacaggtggcaaaacggaagcccagagaaactgtgtgtcttgtccaaatctcactgagtccccagcacatgctggggagaggtgatggcacggactgaggatacagtggcaaaggcagtgcgcacctatcaattccagggccggcaaaagggccagttctgggttctggcatcccaagctccctgtccctggcggcagtctcttccttccctttcagcacctcattatcctggtatctaggaagagcgcagggccccaggcccacagcgtctgcatcccgagaggtgaaaatctaaatgccactgaggcagctctcgggtgtccccagcaccacacagcccatttggggacaagatgacaaaacttgccatccttcataaggctggccccgggtctccaagcccaaactatctacgtcagtgccaacgtgaccagcagcaaaccaatctttcacctcaatcacttcctctcctcccctctaccccctccccgccagtgatcaagtcctcccccaccccaagagaggcagggggtcacattccagggggtgtgggccatctggcagtagcctgaaccccggaaaactgctttccggaaattctctcctgctagaattttattcccaggtctgaccccagcctggggtgtgaatgcccccttctcggccacctttccagctccggagacagccattcccgcagatccctggcgtgcctattccaaggcctgggccgcgcagcaccaaactgccctcccctaggagaccccagggcgccctctggcggacgcgggccgtaggttggggccatcgttccacctcgagctctccagcccggccgagagcacctggcccaacccacaccggaccaaccaggtttgcagaccccagtgcatgaaagcgttttcataggggaggggaggggggctccacactcgcgtcagggttggcaaagaacctggcagtgggccgaggctgggtagctgccacgaaggggcgtcccctgcagccctgggtctcccctctggaatctgcccgcaggtgactgtcgcacgtctccaacctccggctcccgggtctgaccccagccccgctgcctggctgtggggtagccccacttccctttcggctggcccgggccgcctgacccgcagttgggccctcggggaggcccgggtggcggcgagggggtgtccccgcggggtccgggaaaaggggaaggggaaccgggaggctctccaggtcggcctcctagaactgcccgctctcccgaagcggcgcccccgcctctgccagaaatcagccttcttagaagggaggggggtggggggaaagtgtgaatgagaagttgggggcggagcgcgcggcggggaggggccgctgccaggaacgcgccgccggggctggcgccgcgcccaccggccgcctcggccgcctctcgtcgcgctttgtctccgcgcgcgtccctcccggtccctgcccctgctcgcggcccgccctcggcgcccgcggcccctccctcaccctccgcgctcagcctttctctgctgcgagtagtgactaaacattacaagaaggccggccgcgcagttccaggaatcggggggcggggcgcggcggccgcctatatacccgcgagcgcggcctccgcggcggctccgacttggactccctgctccgctgctgccgcttcggccccgcacgcagccagccgccagccgcccgcccggcccagctcccgccgcggccccttgccgcggtccctctcctggtcccctcccggttggtccgggggtgcgcaggggg

SEQ ID NO: 394 - DNA, 498 bp, synthetic construct
  source 1..498: mol_type = other DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccag

SEQ ID NO: 395 - protein, 166 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 396 - DNA, 699 bp, synthetic construct
  source 1..699: mol_type = other DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctg

SEQ ID NO: 397 - protein, 233 aa, synthetic construct
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 398 - DNA, 1623 bp, synthetic construct
  source 1..1623: mol_type = other DNA
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagt

SEQ ID NO: 399 - protein, 541 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 400 - DNA, 1509 bp, synthetic construct
  source 1..1509: mol_type = other DNA
atggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcc

SEQ ID NO: 401 - protein, 503 aa, synthetic construct
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 402 - DNA, 1653 bp, synthetic construct
  source 1..1653: mol_type = other DNA
atggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 403 - protein, 551 aa, synthetic construct
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 404 - DNA, 534 bp, synthetic construct
  source 1..534: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 405 - protein, 178 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 406 - DNA, 459 bp, synthetic construct
  source 1..459: mol_type = other DNA
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagc

SEQ ID NO: 407 - protein, 153 aa, synthetic construct [sequence omitted]

SEQ ID NO: 408 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaatggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 409 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 410 - DNA, 708 bp, synthetic construct
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctggcaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 411 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWQASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 412 - DNA, 597 bp, synthetic construct
  source 1..597: mol_type = other DNA
atgaacagcttcagcaccagcgcctttggacccgtggccttttctctgggactgctgctggttctgcctgccgcttttcctgctcctgtgttcaccctggaagatttcgtcggcgattggagacagaccgccggctacaatctggaccaggtgttggagcaaggcggcgtgtccagcctgtttcagaacctgggagtgtccgtgacacccatccagagaatcgtgctgagcggcgagaacggcctgaagatcgacatccacgtgatcatcccttacgagggcctgtccggcgatcagatgggacagatcgagaagatctttaaggtggtgtaccccgtggacgaccaccacttcaaagtgatcctgcactacggcaccctggtcatcgatggcgtgacccctaacatgatcgactacttcggcagaccctacgagggaatcgccgtgttcgacggcaagaaaatcaccgtgaccggcacactgtggaacggcaacaagatcatcgacgagcggctgatcaaccccgatggcagcctgctgttcagagtgaccatcaacggcgtgacaggatggcggctgtgcgagagaattctggcc

SEQ ID NO: 413 - protein, 199 aa, synthetic construct
MNSFSTSAFGPVAFSLGLLLVLPAAFPAPVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 414 - DNA, 2000 bp, synthetic construct
  source 1..2000: mol_type = other DNA
aaggtctctctctgttacccaggctggagtgcaatggaatatgcatagcttactacagcctctattcttgggctcaagcaatccacctacctcagcttcccaagtagataggactacacgtgcaccaccatgctctgctaattaaaaaaacaaaatctgtagagatggagctttgctgtgttacccaatctggtctcaaattcctggcctcaagtagtcctcctgcctcagcctcacaaagcactaggattacaggcgtgagccattgtgcctggccttactgtagaactttataattcattatcttctttacccaggtgtacatagtattagtgaaataaaatacattaaattaaagccatttttaattttttggtatactatggaattaggcatatttgaaattacttctcatatgacattgtaaacatagccaaattattatcattttcttcctgtagcaataagtctgaacataaaatcttccattttcttatacaaaatgaagctttaaaaaatcaagagcatttaatttgtttcatatataggccttctcaactgcgatcttgccccagagaagagaaaaaactacacagaaaccttaagcattaagacacattattgtaactaagaaagagcataacgttaatagtggctttttctaaggtgtaaaataatgaatttttaagatacttttccttatttttcatattttgtatattaaatatataccacttctatcatttaaaatatctattatgtttttaaaataaaggctttctttccgctttctttcaggccagaggtaaaagaaacagagaagaagagataataattctaacagaaatattacagcctttacaacttgcatgcatacagttatacattaagagactgaaaaaggcagtttcttttactgctaattctagtagtgatgaaactcaggctcaagtattcaggtgacttgtttgaggtcacacagtgcataaatggttgcacctgagctgaaatatgagttgtccaactcttgtaagtgcatgctccatcacatcatcctgagtcagtaatgaggatgccattgtacccttcaagcagccctggagagtttatctctgcatgctggagctaataataatagtgagtatttatatagtccacactaggtaccacattctgctttaagtgctttacaaataataactcatttcatccatacagcaacttcatggggtcgataccattactatcatcctgttttacatataaggaaactgaggttacataactaggccctagatcactcagctagaggtaagactgggatacaagcagactgacttcagagcccatgcttcaccatcacattaagtagcctgctgatttgtctttatttgttatgttaacaggaaaggattgctttcctttagtgcaaggtaagccacaaggcagattataggaaatagtctacaaaaactgaagggagcagcatcaattttcccaaggaattttttttcctttatacttctttatcattagagcccaatttggacaaattcttttccactctgagcctcagtcaatgtttggctcatttcatgaaggaaactgatggtagaggcaagagtacactgctcagcagaccctttgtccaagggtaatttcattggcctccaccattgtttgcctgttaaactgtggcttacaacatttcaaggagcaaagacaaaatgttacacttgtactgtggtgaaactttccatttcaaagaaatagattccagcaggcgtaaagctacctggaagcaaagggtttgttatggggttattataaaatagaaaaacaaaaactcaatctggaacatgacccctttgtcgtacacatgcattgcatctggcctgctgtacaatgtggaccactggcagagactgtgttcacacatggaacacaggagagagctactctaagaagaaaaagttaggaaaactagattcttttctttt

SEQ ID NO: 415 - DNA, 2000 bp, synthetic construct
  source 1..2000: mol_type = other DNA
ggagaagaaggagtcatagagaaaggcttttcatcagtcatggacctaaaagcgtgctctgtaatggtggacttcagacagagatagagagatcctagatgagtcatcctgggatgcaggatgcagttgcttcttttgttttgagaatgcttggctgcttgctccattcctactcatcagtagaagcttgtcttagtgtttcattttcaaggacctcttgatataaactacggtttgaaggacattccgttcctaagtgggacaggagcagagcctgggctgccatgaccagttctgacatatctcaggttgctgcattaccagagcattccaaagttattcctgagaactacaagtgagaaaggttggaaaactgggtcagttcaaggcctcccaaagaaccatcctgcagctatcacgagatatgtctgtctgtctgtctcttaagtagagaaggcagagattaaaaataaaaaagattggaacaacaaagatatagggaaaaataattttggaagctacctttgtgaaatctgtagaaagatgtagagaagaggagctggtaacagccatgtctggactcccggtcaccctattaccacaacctgttaccccaaccccatcacttcacataagaagcaaagttcatgctagcctagtagcccatttcctccaagacaattcccatcaagcctggccaggtggctcagcccagaccgaagcatggaggatgtgcagtgtgaaaaagcacagactgagatgaccatgcagaaaagatttgtataagaaagctggggccacccctgtagccttgtttttataatactttcctgtgtcaaaaaaattaatgaagttaatttagaatgagaaatgatacataatgcctataggtatctctgagaagattgctacagagactaagaggatcatagaccttccttctggaatgttcctgttcctatctgtaggatgaatgagtggaatcagtgactcagacaaagccactgttgcacaactgtatgcatgcacagtgaactctgcagactctgcagatggttcctgtaaatagcgcttaaaaccacagtggaaaagccacatgctcctgatacacttggggggacataaagggaaacccacacacccaaggaggagggctctggccatgcaaggctgaaggcccagtgtactgatatgaaaactgaagctcagacagaagaggcgacgttgctttcttaaccactggatttgcagtgctgtggttctcagaagaggccaggacaggttaatccactcacttgggaggcaggtacccacatttctctccagctgttacctccaacctttagttccaccggtcaaagaagatattttgttttgccttgtcctttgctaaagtgatggtacttgtttgctttctctctgacagaaaatggcagctttggttgttccctcttcctgccaattcctgttgttttcagtcattttaattttagccttcttctggaaggtgcgagtggtatctcagcatgatttcctatttccctcatgaaaaatgatatcaaatactttagagtttcttcatcaaacttttgtgtttcttctcttgtgcattttctattcaagtcctatgcctacttttaaaaatttctttacatttttattgataaataaaatgtaagcattttaatgcttaatgaaatgcttaatgttgtgagcatttctatataaactagaggcaagtttttttcagttgaatgttattaataattttgaacagtctcctcattttatcactggtgtcttttgatgagcagagttttcatgtttaatgaaaagtaatctacactttttttcttttttcattagcattttaatttgctacccaaaacattcttgactactcctatttcatgaagctattcttctgttttctcttaaatgactcagcttttcacattttgacttagcttttacatttcttgacttaacttttacattat

SEQ ID NO: 416 - DNA, 2000 bp, synthetic construct
  source 1..2000: mol_type = other DNA
aaggagtcatagagaaaggcttttcatcagtcatggacctaaaagcgtgctctgtaatggtggacttcagacagagatagagagatcctagatgagtcatcctgggatgcaggatgcagttgcttcttttgttttgagaatgcttggctgcttgctccattcctactcatcagtagaagcttgtcttagtgtttcattttcaaggacctcttgatataaactacggtttgaaggacattccgttcctaagtgggacaggagcagagcctgggctgccatgaccagttctgacatatctcaggttgctgcattaccagagcattccaaagttattcctgagaactacaagtgagaaaggttggaaaactgggtcagttcaaggcctcccaaagaaccatcctgcagctatcacgagatatgtctgtctgtctgtctcttaagtagagaaggcagagattaaaaataaaaaagattggaacaacaaagatatagggaaaaataattttggaagctacctttgtgaaatctgtagaaagatgtagagaagaggagctggtaacagccatgtctggactcccggtcaccctattaccacaacctgttaccccaaccccatcacttcacataagaagcaaagttcatgctagcctagtagcccatttcctccaagacaattcccatcaagcctggccaggtggctcagcccagaccgaagcatggaggatgtgcagtgtgaaaaagcacagactgagatgaccatgcagaaaagatttgtataagaaagctggggccacccctgtagccttgtttttataatactttcctgtgtcaaaaaaattaatgaagttaatttagaatgagaaatgatacataatgcctataggtatctctgagaagattgctacagagactaagaggatcatagaccttccttctggaatgttcctgttcctatctgtaggatgaatgagtggaatcagtgactcagacaaagccactgttgcacaactgtatgcatgcacagtgaactctgcagactctgcagatggttcctgtaaatagcgcttaaaaccacagtggaaaagccacatgctcctgatacacttggggggacataaagggaaacccacacacccaaggaggagggctctggccatgcaaggctgaaggcccagtgtactgatatgaaaactgaagctcagacagaagaggcgacgttgctttcttaaccactggatttgcagtgctgtggttctcagaagaggccaggacaggttaatccactcacttgggaggcaggtacccacatttctctccagctgttacctccaacctttagttccaccggtcaaagaagatattttgttttgccttgtcctttgctaaagtgatggtacttgtttgctttctctctgacagaaaatggcagctttggttgttccctcttcctgccaattcctgttgttttcagtcattttaattttagccttcttctggaaggtgcgagtggtatctcagcatgatttcctatttccctcatgaaaaatgatatcaaatactttagagtttcttcatcaaacttttgtgtttcttctcttgtgcattttctattcaagtcctatgcctacttttaaaaatttctttacatttttattgataaataaaatgtaagcattttaatgcttaatgaaatgcttaatgttgtgagcatttctatataaactagaggcaagtttttttcagttgaatgttattaataattttgaacagtctcctcattttatcactggtgtcttttgatgagcagagttttcatgtttaatgaaaagtaatctacactttttttcttttttcattagcattttaatttgctacccaaaacattcttgactactcctatttcatgaagctattcttctgttttctcttaaatgactcagcttttcacattttgacttagcttttacatttcttgacttaacttttacattatgctctta

SEQ ID NO: 417 - DNA, 2000 bp, synthetic construct
  source 1..2000: mol_type = other DNA
catcctgggatgcaggatgcagttgcttcttttgttttgagaatgcttggctgcttgctccattcctactcatcagtagaagcttgtcttagtgtttcattttcaaggacctcttgatataaactacggtttgaaggacattccgttcctaagtgggacaggagcagagcctgggctgccatgaccagttctgacatatctcaggttgctgcattaccagagcattccaaagttattcctgagaactacaagtgagaaaggttggaaaactgggtcagttcaaggcctcccaaagaaccatcctgcagctatcacgagatatgtctgtctgtctgtctcttaagtagagaaggcagagattaaaaataaaaaagattggaacaacaaagatatagggaaaaataattttggaagctacctttgtgaaatctgtagaaagatgtagagaagaggagctggtaacagccatgtctggactcccggtcaccctattaccacaacctgttaccccaaccccatcacttcacataagaagcaaagttcatgctagcctagtagcccatttcctccaagacaattcccatcaagcctggccaggtggctcagcccagaccgaagcatggaggatgtgcagtgtgaaaaagcacagactgagatgaccatgcagaaaagatttgtataagaaagctggggccacccctgtagccttgtttttataatactttcctgtgtcaaaaaaattaatgaagttaatttagaatgagaaatgatacataatgcctataggtatctctgagaagattgctacagagactaagaggatcatagaccttccttctggaatgttcctgttcctatctgtaggatgaatgagtggaatcagtgactcagacaaagccactgttgcacaactgtatgcatgcacagtgaactctgcagactctgcagatggttcctgtaaatagcgcttaaaaccacagtggaaaagccacatgctcctgatacacttggggggacataaagggaaacccacacacccaaggaggagggctctggccatgcaaggctgaaggcccagtgtactgatatgaaaactgaagctcagacagaagaggcgacgttgctttcttaaccactggatttgcagtgctgtggttctcagaagaggccaggacaggttaatccactcacttgggaggcaggtacccacatttctctccagctgttacctccaacctttagttccaccggtcaaagaagatattttgttttgccttgtcctttgctaaagtgatggtacttgtttgctttctctctgacagaaaatggcagctttggttgttccctcttcctgccaattcctgttgttttcagtcattttaattttagccttcttctggaaggtgcgagtggtatctcagcatgatttcctatttccctcatgaaaaatgatatcaaatactttagagtttcttcatcaaacttttgtgtttcttctcttgtgcattttctattcaagtcctatgcctacttttaaaaatttctttacatttttattgataaataaaatgtaagcattttaatgcttaatgaaatgcttaatgttgtgagcatttctatataaactagaggcaagtttttttcagttgaatgttattaataattttgaacagtctcctcattttatcactggtgtcttttgatgagcagagttttcatgtttaatgaaaagtaatctacactttttttcttttttcattagcattttaatttgctacccaaaacattcttgactactcctatttcatgaagctattcttctgttttctcttaaatgactcagcttttcacattttgacttagcttttacatttcttgacttaacttttacattatgctcttatattaatactgagttaatttgcattgatggtgtgtgggagtattcaaggcagattatctcttttaataaatctacagtgatctaagcaatatttgta

SEQ ID NO: 418 - DNA, 2000 bp, synthetic construct
  source 1..2000: mol_type = other DNA
gaagtctgcaagataggaaaagaaggatcttctcttcggttattcaggagttcctacagccattattttgcctttcctgaccaaatagctggcacttgacggctgtagcatgtaaagactgacttgcccactcacaggttttcttcctcttttcttactgaagagcctgtccttgaaaaaaacattccaggaagaaatctttcttgggccacagagccctgtggtttgatggaaaagaacgaggcttcaagactggctctgctattacttgtagtgtgacattgggcatgtcttaaatgccttccttgggcccctcagcatctttcacctacatatccaaatttcacacaaccttggacttctacaattggaaaggattttaaataatcacttagtaccatcgtctattttactcacaaggcaattaaagccagagtacctgggcttgttcaagattacatgacaaattattgtcagggctagcatgatgaacccagggctctcaattcccagtcagtgtccttttcaaccgttttgcgactctctttggctctgagccccccttgtcctgttaatcagtgccacacattatcgaactcttgtttcctgtgttaatcttgtctccccaaattgtgagattcttgaaagcaggagccactgttttatgcggagggactccataaatattcaccaatgattcattaggaaagcaactattgggggaatcccacttggtcatttccaaaatggaaagcagcatcttggaagtgcaggtccccaaatgtctagggaggtgacaagaaggcaagcaaagaatcaaagaatactgagcacagtgtcctgttgcaatacaggacttagtttgccggccctctggttcctttcctggtctttcagatggagctggagctacttcagcagggctaagaataatcaagtccaggtggcctgcccttccatagcagaaagggacagacaatagagcaatgaaattaccactgttttctacacagaggtgtgtggaaagtgaagagaggggaagttaagaaatttgctgacccaattgggtgggggggggctcttaaaaaggtttatgtagaaattgtcccatctgtgcctccatatttaaaaccttgtccaaaattctaattgaactcacaaagcacaactgtgcatatatttagttcgttatggagtttatggggcagagggtccaggctccttttttccccatgcaaaaagcatgggctcaagctttcttcttttcccctgttgctcaaataaatagtgttctttgctcaaaccccctttccctcctccttctgcaatctcagcgcctagcgcaaatctgttttcttcattgtaacctcagcttcaccgcaattaattttttttccctctggtcacaagataattcctgacgccagtgagtctggaggtcagacgaacagcaaattggggaacaaggcggcactaattccttacaagtttcccttgaaaaatctttcgcttaaaaaaaacggggggtggggggagcttctttgctgttcagggatttatgacctcggaggagctgtggctcgaaccagtgttgggctaaaggcggactggcagggggcagggaagctcaaagatctggggtgctgccaggaaaaagcaaattctggaagttaatggttttgagtgatttttaaatccttgctggcggagaggcccgcctctccccggtatcagcgcttcctcattctttgaatccgcggctccgcggtcttcggcgtcagaccagccggaggaagcctgtttgcaatttaagcgggctgtgaacgcccagggccggcgggggcagggccgaggcgggccattttgaataaagaggcgtgccttccaggcaggctctataagtgaccgccgcggcgagcgtgcgcgcgttgcaggtcactgtagcgggacttcttttggttttctttctctttggggcacctctggactcactccccagc

SEQ ID NO: 419 - DNA, 2000 bp, synthetic construct
  source 1..2000: mol_type = other DNA
gaatcgcttgaacccgggaggtggaggttgcagtgagcccagattgcaccactgcactccagcctgggcgacagagcaagactctgtctcaaagaaacaaaccaaccctgaatgtgattatatacataattcaattaaatgtatttgcttctgaaatatatataaatgtaaattaggcagtcacttttgtatatgatttatttatatttgaaagccacaaatgacccatttaaactattattttcataagccagtgaaacaatgtctgagaaacatttttgttttgtctgttctgttctataaccatcattttttttttcagtcatgtacagccttagtgacaaagaaactttggtcctctgtcctacattttcactatctttttccctccggtcaggataatctcaaatttacatgttaaaaacaatcagtaagagaactacatcacatttctaataggatggaaacttttcaactttatcacaaagacaacgaatgtggaggctttccgtttgaagataaaactattcatttaaaaaattttaaaaattacaatgtttccagtagcttctttttgaattactaacatattccacactctagtaacggtttggccagctaatcgttagtttctgctttaaaatgttctaaattcctgttctacttttgaaaaatgacaacataaatgtttggagggttattttctgcttaatgaaagatctagaaacatattttattctaagaaagaattccacttgcctttaaataaagatataccttttgaccaaacaatcagattttctttttcttttttttcttttcttttttttttttgagatggagtttcgcgtctgtcgcccaggctggagtgtagtggtgcgatcctgactcactgtaacttccacttcccaggttcaaacgattctgttgcctcagcctcctgagtagctgggcttacaggtgtgcatgatcacacccggctaacttttgtatttttagtagagacgggtttttgccatgttgaccaggctggtttcaaactcctgacctcgggtgatctgactgcctcggcctcccaaactgctgggattgcaggcgtgagccattgtgcctggccagattttctttttctagcaaggggacccacttaaacttgaagaggaccgggatggttgaggctgggcagcaaggctttactgcaaatcctttaccactgttttttctggctttctagagaacgttctagcaaaaggtttctagaactttctccttcctggcctgactgacattccctcttaggtgtagcctccttttcacttttcttctgcctggaggaaatgaagctccacggaactttctgttgaaactttccaagaaaaaaaagaaaggctctaagcactgaatgtggaaactgaaggggatgagcttcaactctgaagtgtttccagcgtaaaactgtcctttccagggcccgtgtggctgtcacttcagagtggaggttgtctgctgagggacccctgactcagctgcttcccaggggaagctccgtcttccggcacaggtaatggcctgcagcttgatctccacccagccccatctgagcaggccgggagctcccaggctgtttcacttctctccttcctgactcctcaccatcaccatcgccctctctcctccccaccccgccactcctctcccacacgtgtccctttctccccttcctctgcgtctgctcttctcagaagttagcttacgaagcaaagttgttactttgaattcctgtttttccagccaccctcatgtgacaggatgtctcctcagtagaggctttccctaaattcaggagccctttaaaagggagggcttcctctgtagttcttttcagctgggcagctctgggaacttggattaggtggagaggcagttggggggcctcgttgttttgcgtcttagttccgccctcctgtccatcaggagaaggaaaggataaaccctgggcc

SEQ ID NO: 420 - DNA, 547 bp, synthetic construct
  source 1..547: mol_type = other DNA
ttatagtattaggtcaatgtgttattattttaaagtgttctgtgttcaacgaggctcagaaaatgctggcctcaagacctctgaggacctctgtcatttccagaaacatgtggactttttgcttcaaaaagcattccttgagtaatctgttctgcggggtacatgacatttatacttctctgagaatatggtgtttgttttgaaatgccttatctgtctctgcaaatgctgtcaatacatctgtgttagcacaggacgttccagaagacaggaagcttacatgtgatctgttctcgtaatatgtctagggctatgacgtattcagtgctaatggacctcagttacagcacaaggagacaaggtggagcaatagcattttgttattgggaactaggctacacttacttccttatgcacaataagaggcatccacctaaagaatgatgcaaaatagacaatcaacacagaaattaggcagacactatcactctaagagcagatgttggagtcgccttgattagactttggatcctggctttcgcactta

SEQ ID NO: 421 - DNA, 946 bp, synthetic construct
  source 1..946: mol_type = other DNA
ataggtttagagccttgctcctgcagcttcaaaaacaaggaggcaaaaattgagattgaatatagcacagagtgtgggtgacaccaaaccacggactgagttgataggctgcagggaaggaagtgaaaagttcaccttgtccttccgtctaacagaggactgctggttttgcagaggtttgcttttttttttttttccattcaataaaaatagctcggtgggacaacatattatagcattcgtggaaataaaaactaggaaccacatcataggatgttctgctccaaacttggcgctgatcccccaggcagctccaagttccctctgattcaggcagcagcgacggaggccctcccctgtcccaggtgggacccggccttcagagaggaagggaggtggtggtatgaggcatcagctgtttcctttgcctgtgggccaagatccttgccatggcagaaatacaactttgtccccagtgtctgccttctgcactgtaaaaagcaagtttccatcacaaagggttgcgattcaggaaaccagggttagaggcaaacgggtcttatttgaaaatggtaaaacctggccaggccttggacttcctgcattggttgctctcctgggacctgggaccagcccagcagggtggggtggggcagcaatgcaggttacaggatgtgaaagtggggactgaggttaggaaaaagaataaaaggctgtgaccagaacacacccagcagaggccatgtgcagttctcttcttcagggtttcctgtgggtacactgagaacattgtgttctggctgagaacttatccggcggagcctgctgcatacgtggcccctgggtcctagtgctgcctcttacctagcctcaggcctggctcccctgcctgcacctgcgtgcagcccactgtatgacccagggaggctgtctccattgtgtcctc

SEQ ID NO: 422 - DNA, 576 bp, synthetic construct
  source 1..576: mol_type = other DNA
gcccatttgagtcccaagatctgccaacagatccttcctccacgtctgaaatgatgttgcgtcaggcagaggcacacagccccgactgtctccagtacagacatttcttcaggccacttcaccttcctctgcttacagctccctccgggcaggcgcggggcctgcctgtgctcatcctgcaggcagagaacattgccaggtcagcagtatacaccgtcccctcagacacaaggagtattatttagtctagaaactctcaaaggaaatatccgcttgcacgtgaaaaatctctggaatttctctgttcctctctgtccaacgtccttgtatctgcaaaacctccttgaaaagctcctgtgtacaactgctgccttgttcgggcccgactgcttcaggcctgaccccagggcacttctgtcccagccagtgtcattcagagcgatttctgctcctccaagacgggctgtgcccatggtgaaggaaagaggaggggtaaggggagaaggaggtggggggatgaggtgaagaacagagagggaggagaaggagggagaggaaggggaggaccctctgaag

SEQ ID NO: 423 - DNA, 574 bp, synthetic construct
  source 1..574: mol_type = other DNA
aattagtgcattggtttatcaggaactcttttcttggtgctctattctgctctccatgaccttggttgcctgagtgatacatcctttaatttgttcctacatcttgcttggattttcccctgctcatctctgtctctgcttatacaggatgtgttttattgccacacagacttagctggtagcctctgtggcatcatccaacactgatgtccctcagtcaactctagaacacccataggtggcctctgtaagagacgtttccaggaacagaatgccaaacatactgtttatcagagttcttgagttgcacaatgggaggaaattgttgcttaggatttcaaaggacaccagagaatttcaacacacacctggaatggtgccctgttgcttgtccagtcaatgccaagttaatgatgggaaggaatagctggtgtgtcacgtgattgccaagcactgtcgcaaatccttcctgaggtcaacactcccaaccccgccaaactctacagctcacaaatcagctgcaagtggagcataaaaaatgcaacattgtggaattaactacttatgggccaca

SEQ ID NO: 424 - DNA, 539 bp, synthetic construct
  source 1..539: mol_type = other DNA
ctatcagcaattcattgagccttgtgatttcctgggtgatagagaaacaggccattgaaacgagtccctagtcctagctcatggcatccaaatagcaagatcagtgacatagactattcaatgtgaactaggaggaaatctgcctcatcaccattaaaaatgatgagactacatggtcagtgggtgttaagcaacattgccaggaaaaacgggcaggccaaggttcagatgggaaatgttggcatgcaatgttgatttaagtaatctggataaaaaatacttttcatgctgagacacgagtcatgaggcgctagaatatagggataacttcctttcaactctttaaaaagtacatatggtaaatatttgctgatttagactatgtagagaagaatcaaaatgaatcagtaaaacatccaaatagggaagtatgggtgtggaaagcatgtattttgagcagaatttaacgaagccgaccaagggttccctatggaaggaactatggaatgaaggactgaacatttgattaaagagcagtt

SEQ ID NO: 425 - DNA, 624 bp, synthetic construct
  source 1..624: mol_type = other DNA
ttgtcttgtcgtgattgcaagtgggatgtggtcaattagtcgagctatgctttatgcatccagacccttataattgaggagctggcattcttgcctgtctgggcaggcaattgctcagcagggagcctcatggccagacaggcaagagggaagagggcaatgtggttgctcaatgagcctttagaacaaattgatccggactgtttgtggttccaaaagccacccagtacttgctatgtggttttccttgtggacaattgggcaatgggagggggtggggacaggaaaccttaacctctgcctagccactcacagcaagatgagaggcaactgctcagtgcgtgtcttcagctcatgccctctttcgcctgtcttggcccatcttcttcagggatgggaaccagcatgatgagggagaagttactcaaggggccgtcttcttggcttcagccctgtggaaggcctagtgggctttggacctcacagccaatcaggtgagtcacttgagcaactgaagaaccaagacagcttatggctgggctccaggatggggaggtcccaggagagggcagagcaggagagaaggctctaaggttaattggaaagaacagaacagtgaaaacc

SEQ ID NO: 426 - DNA, 246 bp, synthetic construct
  source 1..246: mol_type = other DNA
ataaatttcttgatagaatttaaatgttaagtgtcctgaaatgtgtttacttctggatcagaaatgagaatgtactgaaacaggaagtcagcttcacagcaggaagcagacctcaaagaaatgtgactcacatagtcttttgaatgtgctccacttggggacacaatgttccccacagcttgcccatctcatccattctaactttcccatgggacaaaaagcatcataacaaagaattagaggaga

SEQ ID NO: 427 - DNA, 410 bp, synthetic construct
  source 1..410: mol_type = other DNA
atcaaaaattttggtcagacatgagatcttctccctgtcagactgtctttttatagacattgtgtcacaggttctgaaatgttgaaagggttttgtttcctgactcaggctacttgattctgcctattcacagaagtctgcagcacccaccgaggctggccacgtgattggatatggcctttctttagctctgcagatatgtggatatattcatttgctagggctgcaataacaaataccacagtctgggttgcttgcacaacagaaatttattgtctcacagctctggagactagaagtccaggatcaaggtcttagccaatttggttccttctgggggctatgagagagacactgttccatgcctctcctagcttctggtggttggctggtttgctggccatctttgg

SEQ ID NO: 428 - DNA, 99 bp, synthetic construct
  source 1..99: mol_type = other DNA
ccacttccgggttcagaccacttccgggttctcgccacttccgggttcgacccacttccgggttcctaccacttccgggttcactccacttccgggttc

SEQ ID NO: 429 - DNA, 90 bp, synthetic construct
  source 1..90: mol_type = other DNA
ctgacctttgtgccatactagtcacgtgccttccgaaaacactgcacattgtttccggtaaataaacaattaaaatcttctgagtcagca

SEQ ID NO: 430 - DNA, 90 bp, synthetic construct
  source 1..90: mol_type = other DNA
tgcacctgttacggttgtatgtcacgtgccttcgggtaacactgcacattgtttccacacatctgcttcctgtgctccactgagtcagca

SEQ ID NO: 431 - DNA, 92 bp, synthetic construct
  source 1..92: mol_type = other DNA
ctgagtcagcaagggtgcttaagtaaacaaatgccgacttattggaaaaactgctaacagtttcattatgactcacggtccacttccggtct

SEQ ID NO: 432 - DNA, 88 bp, synthetic construct
  source 1..88: mol_type = other DNA
tttcacacctttcgtttaacaatgtgcagtgttatgtatctgagtcagcaaataaccaccacttccggtctagacgcgattttttcca

SEQ ID NO: 433 - DNA, 95 bp, synthetic construct
  source 1..95: mol_type = other DNA
acgtcaatgggtggagtatttacggtaaactgcccacttggcagtacatcaagtgtatcatatgccaagtacgccccctattgacgtcaatgacg

SEQ ID NO: 434 - DNA, 90 bp, synthetic construct
  source 1..90: mol_type = other DNA
aggcggaagtgagctctgagtcataatgaaactgaccagatttccaggaaaacgatgacctgcagtgacctctgtgtatattgcacaatc

SEQ ID NO: 435 - DNA, 91 bp, synthetic construct
  source 1..91: mol_type = other DNA
tattgcacaatctaagcatttttcctggaaaagattgcctgatgactcatctattatgctatcacgtcaccactgggtctcacttccgcct

SEQ ID NO: 436 - DNA, 90 bp, synthetic construct
  source 1..90: mol_type = other DNA
tattgcacaatcagctagcatttccaggaaaaacggaacaaggcggaagtgttttcgtcagatgactcatctatcggcgaaacagctgtt

SEQ ID NO: 437 - DNA, 92 bp, synthetic construct
  source 1..92: mol_type = other DNA
tattgcacaatctagctatatttccaggaaaagtatagcatgcgtgggcgtagatctgatgtcacgtgacttcacgatctcacttcctgctc

SEQ ID NO: 438 - DNA, 92 bp, synthetic construct
  source 1..92: mol_type = other DNA
gagcaggaagtgagtcgccattcctttgatcttttgactgcagtcacgtgactgaggagcatttccaggaaaaccgaactaaggcggaagtg

SEQ ID NO: 439 - DNA, 93 bp, synthetic construct
  source 1..93: mol_type = other DNA
tgacctgcagtgacctctgtgagtcataatgaaactgaggtctcacttcctgctctccgtttgatgagtcatcttcgcgcagattgtgcaata

SEQ ID NO: 440 - DNA, 92 bp, synthetic construct
  source 1..92: mol_type = other DNA
ccgaaaccgaaactaatacaatcaaaggtcagttgagatagaatggaaaaaccagccagagtcataatgaaactgtacacatatgtaaatat

SEQ ID NO: 441 - DNA, 93 bp, synthetic construct
  source 1..93: mol_type = other DNA
aaggtgtgaaatgctagataattgtttatttagctacgaaagtaaacaaaaaatgtaaagatggaaaaaccctatagagtcataatgaaactg

SEQ ID NO: 442 - DNA, 94 bp, synthetic construct
  source 1..94: mol_type = other DNA
gagtcataatgaaactgaaggtaaaagataagaaaaaaactccgaaaccgaaactaagtgtttcaaaggtcagaaattcccaaaataaacaatt

SEQ ID NO: 443 - DNA, 95 bp, synthetic construct
  source 1..95: mol_type = other DNA
aatggcccgcctggctgaccgcccaacgacccccgcccattgacgtcaataatgacgtatgttcccatagtaacgccaatagggactttccattg

SEQ ID NO: 444 - DNA, 408 bp, synthetic construct
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 445 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 446 - DNA, 25 bp, synthetic construct
  source 1..25: mol_type = other DNA
tctagagggtatataatgggggcca

SEQ ID NO: 447 - DNA, 65 bp, synthetic construct
  source 1..65: mol_type = other DNA
taggcgtgtacggtgggaggcctatataagcagagctcgtttagtgaaccgtcagatcgcctgga

SEQ ID NO: 448 - DNA, 63 bp, synthetic construct
  source 1..63: mol_type = other DNA
ttcgcatattaaggtgacgcgtgtggcctcgaacaccgagcgaccctgcagcgacccgcttaa

SEQ ID NO: 449 - DNA, 81 bp, synthetic construct
  source 1..81: mol_type = other DNA
aggtctatataagcagagctcgtttagtgaaccgtcagtccgcctggagacctcgagccgagtggtcgtgcctccatagaa

SEQ ID NO: 450 - DNA, 85 bp, synthetic construct
  source 1..85: mol_type = other DNA
tctagagggtatataagcagagctcgtttagtgaaccgtcagtccgcctggagacctcgagccgagtggtcgtgcctccatagaa

SEQ ID NO: 451 - DNA, 128 bp, synthetic construct
  source 1..128: mol_type = other DNA
cggatcaacttaggcgtgtacggtgggaggcctatataagcagagctcgtttagtgaaccgtcagatcgcctggaggatccgcgtcaagtggagcaaggcaggtggacagtcctgcaggggagctacc

SEQ ID NO: 452 - DNA, 126 bp, synthetic construct
  source 1..126: mol_type = other DNA
cggatcaactttcgcatattaaggtgacgcgtgtggcctcgaacaccgagcgaccctgcagcgacccgcttaaggatccgcgtcaagtggagcaaggcaggtggacagtcctgcaggggagctacc

SEQ ID NO: 453 - DNA, 144 bp, synthetic construct
  source 1..144: mol_type = other DNA
cggatcaactaggtctatataagcagagctcgtttagtgaaccgtcagtccgcctggagacctcgagccgagtggtcgtgcctccatagaaggatccgcgtcaagtggagcaaggcaggtggacagtcctgcaggggagctacc

SEQ ID NO: 454 - DNA, 148 bp, synthetic construct
  source 1..148: mol_type = other DNA
cggatcaacttctagagggtatataagcagagctcgtttagtgaaccgtcagtccgcctggagacctcgagccgagtggtcgtgcctccatagaaggatccgcgtcaagtggagcaaggcaggtggacagtcctgcaggggagctacc

SEQ ID NO: 455 - DNA, 165 bp, synthetic construct
  source 1..165: mol_type = other DNA
cggatcaacttctagagggtatataatgggggccagaacacatcgctaagcgaaagctaagctccagtacaggggcttttggtgaccggtcagaggaaaggtgaatccaatgggatccgcgtcaagtggagcaaggcaggtggacagtcctgcaggggagctacc

SEQ ID NO: 456 - DNA, 1181 bp, synthetic construct
  source 1..1181: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 457 - DNA, 2000 bp, synthetic construct
  source 1..2000: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcttctaagtccaattcacgacaagagtagctgggattacaggcacatgccaccatgcccagctaatttttgtattttcaatagagactgggtttcaccatgttgcccaggctggtcttcaactcctgacctcaagtgatccgccctcctcagcttcccaaagtgcgccttcataaggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatatctcgctaataggagtaagatacatatagatatgataggtcttcattctgctgcaagacctatactataactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggacgagttcgataatacactgttactatgtttctaatttttcatgtgcttctatttttttcctacttcagagccattgactaatagttgagtataacacaggttgtgtttccgggctgctgaaacatgacactaatattttcaaagaactgtggaagcctaaaaggtgtctgtataaagataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 458 - DNA, 1890 bp, synthetic construct
  source 1..1890: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggctggagtgcaatggtgcaatcttggctcactgcaacctctgcctctcgggctcaagcgagtagctgggattacaggcacatgccaccatgcccagctaatttttgtattttcaatagagactgggtttcaccatgttgcccaggctggtcttcaactcctgacctcaagtgatccgccctcctcagcttcccaaagtgcggcgtgagccaccacaccggggggtaggatagatttagtgagatgactggataaacggaatcaagaaaaagctttgtcaaaaacttatgcttcttaaaaacttaatcctgggacagaatcatctaaaacgttgttccatgtcttcactttgactcacccataaaaacttcaagtacaaagaatgaaaaataaccacatattttctaatgctcaatatatagatatgataggtcttcaaactgattcccaaagtattagcctcatgaatcatgtagtcataagaaacacagtcattgtattctctttgctgtataattttggtttcagttttccttacatttcctattcaaggaacattttcctgtaaaatgacaggttgaagaaaacagccataatttagtagagaatagcgcgagagctattctagactgtaacgaaagccatatgctatcacaatttaatttatttcaagtactaataagctgatgacaaaacagcgatgtcttttagtttactcacacgaactatttctcttttctccttttgatcatctagaggaacgggcaacttggtttcttctttagcttccttgttctcattaagattgaacaatgcctctaaagtgaaccacagacttgcatgcaagctgaaaacctttaccaaatgcagtcttaatttgtactttgagaaaaacattttcaaggtattttatccttttctccaacttttgacatattacaaagtacccaaatatgccagactgttgcctcatcagccccccgcagtcaggtacagttagatgcaaggcaatcttcctaaaagttacttattagagatgtgagaagggcaaatgctatcattggaaaaactgacaaaagtcccaataggagttactatgtttctaatttttcatgtgcttctatttttttcctacttcagagccattgactaatagttgagtataacacaggttgtgtttccgggctgctgaaacatgacactaatattttcaaagaactgtggaagcctaaaaggataactaaatgagagtttaggactgcagccttcattttcattcaaagatttaaaagtttccataaagtaaaatgttcttctccggccacctgttttcatagttctgtgttttccttcaggcctttctggcttcctatatggcagtaagaaaatgatgtgcttaatgattacaaatttcatatggaatacgaactttcagtttgtacatatgatgcacagagatgcttttgtggttttattggttttcatattacaaacaaagaaactagaaaatgaaaccattccaaaagtggaagtaatttctcactgcccctgtgataaactgtggtcactggctgtggcagcaactattataagatgctctgaaaactcttcagacactgaggggcaccagaggagcagactacaaga

SEQ ID NO: 459 - DNA, 205 bp, synthetic construct
  source 1..205: mol_type = other DNA
ggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcaactagttctagagggtatataatgggggccaacgcgtaccggtgtcgccacc

SEQ ID NO: 460 - DNA, 511 bp, synthetic construct
  source 1..511: mol_type = other DNA
ggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcaactagttctagagggtatataatgggggccaacgcgtaccggtgtcgccacc

SEQ ID NO: 461 - DNA, 1594 bp, synthetic construct
  source 1..1594: mol_type = other DNA
ggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcactgtaatatcatccgctctttactgaaataacataataatataggaagaatacaaggactatagaaatacagattagtgtttgaacccttgcttaccagctactactaatatgattgtggatgaggtagcttcttacttattaacggggatactaatagaggtggttccttgatcggccaatatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 462 - DNA, 2000 bp, synthetic construct
  source 1..2000: mol_type = other DNA
tctccttcaccttccaccatgagtaaaagcttcttgaggcctcttacctactaggttaaggtgccatacttcttgtatagttggaggaactatgaatcaattaaacactcgaattcagaaaattacccagcctcaagtattcctttatagtatgcaaaagagactaaaacagcaaatgaaaagagatctgtctctgaaagaacttattgattctagccttacagcctaaaaaagtcagattgacactgggtaagaagcaaggaggtcaggtcccaagatgaagtcctgcctgtggtcagcaaaggggcaccaaggtgtctgggacagtcctggccctggctttgactctacggaagtagcttgtttaaaacctatagtctcttcggagtcgttctactagtacaaaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcactgtaatatcatccgctctttactgaaataacataataatataggaagaatacaaggactatagaaatacagattagtgtttgaacccttgcttaccagctactactaatatgattgtggatgaggtagcttcttacttattaacggggatactaatagaggtggttccttgatcggccaatatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 463 - DNA, 1850 bp, synthetic construct
  source 1..1850: mol_type = other DNA
tctccttcaccttccaccatgagtaaaagcttcttgaggcctggtgccatacttcttgtatagttggaggaactatgaatcaattaaacaaattacccagcctcaagtattcctttatagtatgcaaaagagactaaaacagcaaatgaaaagagatctgtctctgaaagaacttattgaaaagtcagattgacactgggtaagaagcaaggaggtcaggtcccaagatgaagtcctgcctgtggtcagcaaaggggcaccaaggtgtctgggacagtcctggccctggctttgggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcactgaaataacataataatataggaagaatacaaggactatagaaatacagattagtgtttgaacccttgcttaccagctactactaatatgattgtggatgaggtagcttcttacttattaacggggatactaatagaggtggttcctatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 464 - DNA, 2000 bp, synthetic construct
  source 1..2000: mol_type = other DNA
tctccttcaccttccaccatgagtaaaagcttcttgaggcctcaccagaagcagatgctggtgccatacttcttgtatagttggaggaactatgaatcaattaaacactcgaattcagaaaattacccagcctcaagtattcctttatagtatgcaaaagagactaaaacagcaaatgaaaagagatctgtctctgaaagaacttattgattctagccttacagcctaaaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcactgtaatatcatccgctctttactgaaataacataataatataggaagaatacaaggactatagaaatacagattagtgtttgaacccttgcttaccagctactactaatatgattgtggatgaggtagcttcttacttattaacggggatactaatagaggtggttccttgatcggccaatatttatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 465 - DNA, 1929 bp, synthetic construct
  source 1..1929: mol_type = other DNA
tctccttcaccttccaccatgagtaaaagcttcttgaggcctcaccagaagcagatgctggtgccatacttcttgtatagttggaggaactatgaatcaattaaacaaattacccagcctcaagtattcctttatagtatgcaaaagagactaaaacagcaaatgaaaagagatctgtctctgaaagaacttattgaaaagtcagattgacactgggtaagaagcaaggaggtcaggtcccaagatgaagtcctgcctgtggtcagcaaaggggcaccaaggtgtctgggacagtcctggccctggctttgggcagggagggaatttcccataggaagggaagagtaaagagagagagagaggtcagagtccaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcactgaaataacataataatataggaagaatacaaggactatagaaatacagattagtgtttgaacccttgcttaccagctactactaatatgattgtggatgaggtagcttcttacttattaacggggatactaatagaggtggttcctatgattttttatttaatagtgataccaaagcaatacatactcagtagaaacctacttcaagttcccataaaatcatctgcttttcactttcagtacagtatttaataacttaaatgagatatttcacacttcagtagtaaatacactttttgttagataattttgtccaactgtatgctaatgtaagtgttctgagcatgtttaaggcaggttaggttaagctatgatgtttggtgggttaggtgtattaaatgcatttctgattttggatattttcagtgtacaatgggtttacagggatgtaaccccatcataagtgaaggagcacctgtacttacttcattaaaatgctgaaacagtaaataaggtaacatttaataatatgttgtgcagttcttgaaatttaagtactcaccaaatattacttttcctttttttgttatttacttacttttcattcatttattaattcatttgtgcatttagtaaacatttataaattatttcctgtgcctgacagcatgctggaacagtgctaaagatacaagttaattaagacacaatcacgacccccaagattcctactcttttctaaagattacagacaagcagacgatgctattgttgaagaaacatgctctgagaggcatttgaaggaagtgtagaggatagaagatggacacataacccaggatggggaggaaaagagttagggaaggctttttgacgaagatactgtttacaccgtgtgttcttataaattcatggtggtggggatagagttggaggaaaaggcatgctcagtggcgtggagatggcagagagattggggtgttcaaggatatgccgggaattcaaggaacgagaattcccatagacacagacacagctagacatagagatctgcagcttaggtttgggctgtgggtatagatccaggtggcttcaacagacaaagatctttcctgagaaaagggaaaagttttcaacacagaaagaccatcccatgtttggaatgaggtttgcaaatagattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatataagctgcatgcaaagtcctttttctgtctctggtttctggccccttgtctgcagag

SEQ ID NO: 466 - DNA, 753 bp, synthetic construct
  source 1..753: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactcaggcggcggtggtagtggaggcggaggctcaggcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtg

SEQ ID NO: 467 - protein, 251 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSGGGGSGGGGSGGGGSGGGGSGGGSLQLLPSWAITLISVNGIFVICCLTYCFAPRCRERRRNERLRRESVRPV

SEQ ID NO: 468 - DNA, 669 bp, synthetic construct
  source 1..669: mol_type = other DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggtcaggcggcggtggtagtggaggcggaggctcaggcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtg

SEQ ID NO: 469 - protein, 223 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGSGGGGSGGGGSGGGGSGGGGSGGGSLQLLPSWAITLISVNGIFVICCLTYCFAPRCRERRRNERLRRESVRPV

SEQ ID NO: 470 - DNA, 1338 bp, synthetic construct
  source 1..1338: mol_type = other DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagggtagcggccagtgtaccaactacgccctgctgaaactggccggcgacgtggaatctaatcctggacctggatctggcgagggacgcgggagtctactgacgtgtggagacgtggaggaaaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctggcaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 471 - protein, 446 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQGSGQCTNYALLKLAGDVESNPGPGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWQASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 472 - DNA, 1509 bp, synthetic construct
  source 1..1509: mol_type = other DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggtcaggcggcggtggtagtggaggcggaggctcaggcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctggcaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 473 - protein, 503 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGSGGGGSGGGGSGGGGSGGGGSGGGSLQLLPSWAITLISVNGIFVICCLTYCFAPRCRERRRNERLRRESVRPVGSGQCTNYALLKLAGDVESNPGPGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWQASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 474 - DNA, 1593 bp, synthetic construct
  source 1..1593: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactcaggcggcggtggtagtggaggcggaggctcaggcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctggcaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 475 - protein, 531 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSGGGGSGGGGSGGGGSGGGGSGGGSLQLLPSWAITLISVNGIFVICCLTYCFAPRCRERRRNERLRRESVRPVGSGQCTNYALLKLAGDVESNPGPGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWQASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 476 - DNA, 450 bp, synthetic construct
  source 1..450: mol_type = other DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacaggg

SEQ ID NO: 477 - protein, 150 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTG

SEQ ID NO: 478 - DNA, 81 bp, synthetic construct
  source 1..81: mol_type = other DNA
tcaggcggcggtggtagtggaggcggaggctcaggcggcggaggttccggaggtggcggttccggcggaggatctcttcaa

SEQ ID NO: 479 - protein, 12 aa, synthetic construct
SGGGGSGGGGSG

SEQ ID NO: 480 - DNA, 138 bp, synthetic construct
  source 1..138: mol_type = other DNA
ttgctgcctagctgggccatcacactgatctccgtgaacggcatcttcgtgatctgctgcctgacctactgcttcgcccctagatgcagagagcggagaagaaacgagcggctgagaagagaaagcgtgcggcctgtg

SEQ ID NO: 481 - protein, 46 aa, synthetic construct
LLPSWAITLISVNGIFVICCLTYCFAPRCRERRRNERLRRESVRPV

SEQ ID NO: 482 - DNA, 84 bp, synthetic construct
  source 1..84: mol_type = other DNA
gttaagtggctagggataacattgaggcactaaagcattattggttctgcagtcaagggtaggatagattgttttttttttttt

SEQ ID NO: 483 - DNA, 80 bp, synthetic construct
  source 1..80: mol_type = other DNA
tttgtggttttattggttttcatattacaaacaaagaaactagaaaatgaaaccattccaaaagtggaagtaatttctca

SEQ ID NO: 484 - DNA, 246 bp, synthetic construct
  source 1..246: mol_type = other DNA
gctcttctaaaaatatgcgaaatgaggtttttagggaggtgtaggtatggctgaagaaaatcaaggtgaatgaagacaagatcaattgagaatgtagtttcagaaatagcaaagaagccaaagtttgaggaagttaagtggctagggataacattgaggcactaaagcattattggttctgcagtcaagggtaggatagattgttttttttttttttgagacggagtctcactctgctgcccaggc

SEQ ID NO: 485 - protein, 21 aa, synthetic construct
IYIWAPLAGTCGVLLLSLVIT

SEQ ID NO: 486 - protein, 27 aa, synthetic construct
IYIWAPLAGTCGVLLLSLVITLYCNHR

SEQ ID NO: 487 - protein, 28 aa, synthetic construct
IYIWAPLAGTCGVLLLSLVITLYCNHRN

SEQ ID NO: 488 - protein, 45 aa, synthetic construct; identical to SEQ ID NO: 106

SEQ ID NO: 489 - protein, 86 aa, synthetic construct; identical to SEQ ID NO: 105

SEQ ID NO: 490 - protein, 10 aa, synthetic construct
GSGSGSGSGG

SEQ ID NO: 491 - protein, 17 aa, synthetic construct
AEAAAKEAAAKEAAAKA

SEQ ID NO: 492 - protein, 18 aa, synthetic construct
KESGSVSSEQLAQFRSLD

SEQ ID NO: 493 - protein, 14 aa, synthetic construct
EGKSSGSGSESKST

SEQ ID NO: 494 - DNA, 184 bp, synthetic construct
  source 1..184: mol_type = other DNA
ggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcaactagttctagagggtatataatgggggcca

SEQ ID NO: 495 - DNA, 490 bp, synthetic construct
  source 1..490: mol_type = other DNA
ggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcaggtttgttgttcatatgttttcttgaaagggcactatttcccagaatccaggtcatctctgggtagggaatcccctgaattagttttttctgagagtatattagatttgccctcacagtaaccccatgctggaaatacccaactccagtagcaactagttctagagggtatataatgggggcca

SEQ ID NO: 496 - DNA, 15 bp, synthetic construct
  source 1..15: mol_type = other DNA
acgcgtaccggtgtc

SEQ ID NO: 497 - DNA, 21 bp, synthetic construct
  source 1..21: mol_type = other DNA
acgcgtaccggtgtcgccacc

SEQ ID NO: 498 - DNA, 1629 bp, synthetic construct
  source 1..1629: mol_type = other DNA
atggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgccagccacggctttccgcctgaggttgaagagcaagccgccggtacattgcctatgtcctgcgcacaagaaagcggtatggaccggcacccagccgcttgtgcttcagctcgcatcaacgtc

SEQ ID NO: 499 - protein, 543 aa, synthetic construct
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPASHGFPPEVEEQAAGTLPMSCAQESGMDRHPAACASARINV

SEQ ID NO: 500 - DNA, 120 bp, synthetic construct
  source 1..120: mol_type = other DNA
agccacggctttccgcctgaggttgaagagcaagccgccggtacattgcctatgtcctgcgcacaagaaagcggtatggaccggcacccagccgcttgtgcttcagctcgcatcaacgtc

SEQ ID NO: 501 - protein, 40 aa, synthetic construct
SHGFPPEVEEQAAGTLPMSCAQESGMDRHPAACASARINV

SEQ ID NO: 502 - DNA, 287 bp, synthetic construct
  source 1..287: mol_type = other DNA
gataagcttgatatcgcctgcagccgaattctcagcctgcatttctgccaggggatgagtcatcacgtccgaacacttccgccttacagctgggagcaggtgcagtcgcgtaatcacgtcaccattatccaaggggattcccctcggatcaacttctagagggtatataatgggggccagaacacatcgctaagcgaaagctaagctccagtacaggggcttttgcccatgaaagagaggaaaggtgaatccaatgggatccgcgtcaagtggagcaaggcaggtgg

SEQ ID NO: 503 - DNA, 81 bp, synthetic construct
  source 1..81: mol_type = other DNA
cggatcaacttctagagggtatataatgggggccagaacacatcgctaagcgaaagctaagctccagtacaggggcttttg

SEQ ID NO: 504 - DNA, 10 bp, synthetic construct
  source 1..10: mol_type = other DNA
cccatgaaag

SEQ ID NO: 505 - DNA, 287 bp, synthetic construct
  source 1..287: mol_type = other DNA
gataagcttgatatcgcctgcagccgaattctcagcctgcatttctgccagggtcactttcactttcttataactgcacctgctccctacgactccggggaattcagtccacaagatgagtcatcagctggctagtcacgtgaccggatcaacttctagagggtatataatgggggccagaacacatcgctaagcgaaagctaagctccagtacaggggcttttgggcggactccagaggaaaggtgaatccaatgggatccgcgtcaagtggagcaaggcaggtgg

SEQ ID NO: 506 - DNA, 10 bp, synthetic construct
  source 1..10: mol_type = other DNA
ggcggactcc

SEQ ID NO: 507 - DNA, 293 bp, synthetic construct
  source 1..293: mol_type = other DNA
gataagcttgatatcgcctgcagccgaattctcagcctgcatttctgccaggggggaaacggaaaccgaaactgtataggggaatcccctacgttcattgcgtgggcgttggagttgtatcacgtcaccttgcgttctcacttcctgctccggatcaacttctagagggtatataatgggggccagaacacatcgctaagcgaaagctaagctccagtacaggggcttttggaggctggacagaggaaaggtgaatccaatgggatccgcgtcaagtggagcaaggcaggtgg

SEQ ID NO: 508 - DNA, 10 bp, synthetic construct
  source 1..10: mol_type = other DNA
gaggctggac

SEQ ID NO: 509 - DNA, 293 bp, synthetic construct
  source 1..293: mol_type = other DNA
gataagcttgatatcgcctgcagccgaattctcagcctgcatttctgccagggtcagtttcattttccaactaagattgtgcaatattccacttagtcacgtgacttggtcccacacttccgcctaagagggaaacggaaaccgaaactgcggatcaacttctagagggtatataatgggggccagaacacatcgctaagcgaaagctaagctccagtacaggggcttttgccttggtggtagaggaaaggtgaatccaatgggatccgcgtcaagtggagcaaggcaggtgg

SEQ ID NO: 510 - DNA, 10 bp, synthetic construct
  source 1..10: mol_type = other DNA
ccttggtggt

SEQ ID NO: 511 - DNA, 295 bp, synthetic construct
  source 1..295: mol_type = other DNA
gataagcttgatatcgcctgcagccgaattctcagcctgcatttctgccaggggggaaacggaaaccgaaactgaagttgcgtgggcgtaggtagtctgcacctgctcccttagaagagcaggaagtgagagtcagaggtcactgcaggtcacggatcaacttctagagggtatataatgggggccagaacacatcgctaagcgaaagctaagctccagtacaggggcttttgggttgcgcggagaggaaaggtgaatccaatgggatccgcgtcaagtggagcaaggcaggtgg

SEQ ID NO: 512 - DNA, 10 bp, synthetic construct
  source 1..10: mol_type = other DNA
ggttgcgcgg

SEQ ID NO: 513 - DNA, 290 bp, synthetic construct
  source 1..290: mol_type = other DNA
gataagcttgatatcgcctgcagccgaattctcagcctgcatttctgccaggggggagcaggtgcagatgaagaacgcccacgcattgatgacttttccaggaaaatcacagtttcggtttccgtttccctaggaaaatgaaactgacggatcaacttctagagggtatataatgggggccagaacacatcgctaagcgaaagctaagctccagtacaggggcttttgagtatcgagcagaggaaaggtgaatccaatgggatccgcgtcaagtggagcaaggcaggtgg

SEQ ID NO: 514 - DNA, 10 bp, synthetic construct
  source 1..10: mol_type = other DNA
agtatcgagc

SEQ ID NO: 515 - DNA, 290 bp, synthetic construct
  source 1..290: mol_type = other DNA
gataagcttgatatcgcctgcagccgaattctcagcctgcatttctgccagggggaaaatgaaactgaatcctctcacttcctgctctaggcagggagcaggtgcagtgatgctgatgactcatcatcgagtaacgaaaccgaaactcggatcaacttctagagggtatataatgggggccagaacacatcgctaagcgaaagctaagctccagtacaggggcttttgtgaatccgggagaggaaaggtgaatccaatgggatccgcgtcaagtggagcaaggcaggtgg

SEQ ID NO: 516 - DNA, 10 bp, synthetic construct
  source 1..10: mol_type = other DNA
tgaatccggg

SEQ ID NO: 517 - DNA, 286 bp, synthetic construct
  source 1..286: mol_type = other DNA
gataagcttgatatcgcctgcagccgaattctcagcctgcatttctgccagggaacagctgtttctcagggaaacggaaaccgaaactgtattaggcggaagtgttgcttatcactttcactttcaacgctaatcacgtcacccggatcaacttctagagggtatataatgggggccagaacacatcgctaagcgaaagctaagctccagtacaggggcttttgcgctcagattagaggaaaggtgaatccaatgggatccgcgtcaagtggagcaaggcaggtgg

SEQ ID NO: 518 - DNA, 10 bp, synthetic construct
  source 1..10: mol_type = other DNA
cgctcagatt

SEQ ID NO: 519 - DNA, 289 bp, synthetic construct
  source 1..289: mol_type = other DNA
gataagcttgatatcgcctgcagccgaattctcagcctgcatttctgccagggcacttccgccttagaggaaacgaaaccgaaactattctatatcacgtcacctatctacaaggggattcccctaagagatcagtttcattttcccggatcaacttctagagggtatataatgggggccagaacacatcgctaagcgaaagctaagctccagtacaggggcttttgggagtaagggagaggaaaggtgaatccaatgggatccgcgtcaagtggagcaaggcaggtgg

SEQ ID NO: 520 - DNA, 10 bp, synthetic construct
  source 1..10: mol_type = other DNA
ggagtaaggg

SEQ ID NO: 521 - DNA, 290 bp, synthetic construct
  source 1..290: mol_type = other DNA
gataagcttgatatcgcctgcagccgaattctcagcctgcatttctgccagggaggggaatccccttgtcgaatgatgagtcatctacgtctaacgaaaccgaaactatactcaggtgacgtgattgctcaaggaaaatgaaactgacggatcaacttctagagggtatataatgggggccagaacacatcgctaagcgaaagctaagctccagtacaggggcttttgcgaagcatagagaggaaaggtgaatccaatgggatccgcgtcaagtggagcaaggcaggtgg

SEQ ID NO: 522 - DNA, 10 bp, synthetic construct
  source 1..10: mol_type = other DNA
cgaagcatag

SEQ ID NO: 523 - DNA, 288 bp, synthetic construct
  source 1..288: mol_type = other DNA
gataagcttgatatcgcctgcagccgaattctcagcctgcatttctgccaggggatgagtcatctggtcagaggtcactgcaggtcattactgtcacgtgacaccagtgatacgcccacgcaatacaacagtttcattatgactccggatcaacttctagagggtatataatgggggccagaacacatcgctaagcgaaagctaagctccagtacaggggcttttgaggcctgccgagaggaaaggtgaatccaatgggatccgcgtcaagtggagcaaggcaggtgg

SEQ ID NO: 524 - DNA, 10 bp, synthetic construct
  source 1..10: mol_type = other DNA
aggcctgccg

SEQ ID NO: 525 - DNA, 286 bp, synthetic construct
  source 1..286: mol_type = other DNA
gataagcttgatatcgcctgcagccgaattctcagcctgcatttctgccaggggtcacgtgacacatctaggaaaatgaaactgaatgggaaacggaaaccgaaactgttagtttcggtttcgttaggttgtgaattccccggcggatcaacttctagagggtatataatgggggccagaacacatcgctaagcgaaagctaagctccagtacaggggcttttgtcagcgggttagaggaaaggtgaatccaatgggatccgcgtcaagtggagcaaggcaggtgg

SEQ ID NO: 526 - DNA, 10 bp, synthetic construct
  source 1..10: mol_type = other DNA
tcagcgggtt

SEQ ID NO: 527 - DNA, 290 bp, synthetic construct
  source 1..290: mol_type = other DNA
gataagcttgatatcgcctgcagccgaattctcagcctgcatttctgccagggtgacctgcagtgacctctgtcaaggggattcccctaatcagtttcggtttccgtttccctcagagtcacgtgactcgtcgaggtaacagctgttcggatcaacttctagagggtatataatgggggccagaacacatcgctaagcgaaagctaagctccagtacaggggcttttgcggtataaggagaggaaaggtgaatccaatgggatccgcgtcaagtggagcaaggcaggtgg

SEQ ID NO: 528 - DNA, 10 bp, synthetic construct
  source 1..10: mol_type = other DNA
cggtataagg

SEQ ID NO: 529 - DNA, 293 bp, synthetic construct
  source 1..293: mol_type = other DNA
gataagcttgatatcgcctgcagccgaattctcagcctgcatttctgccaggggggaaacggaaaccgaaactgatgaaggcggaagtgtgttcgacaccggggaattcatatcaaatattgcacaatctagtctggaaaatgaaactgacggatcaacttctagagggtatataatgggggccagaacacatcgctaagcgaaagctaagctccagtacaggggcttttgccaagacacgagaggaaaggtgaatccaatgggatccgcgtcaagtggagcaaggcaggtgg

SEQ ID NO: 530 - DNA, 10 bp, synthetic construct
  source 1..10: mol_type = other DNA
ccaagacacg

SEQ ID NO: 531 - DNA, 287 bp, synthetic construct
  source 1..287: mol_type = other DNA
gataagcttgatatcgcctgcagccgaattctcagcctgcatttctgccaggggaaagtgaaagtgaatttcgtccggggaattctcagacttagatgagtcatcttccatagtacgcccacgcattagtattaaacagctgttcggatcaacttctagagggtatataatgggggccagaacacatcgctaagcgaaagctaagctccagtacaggggcttttggctgtggcagagaggaaaggtgaatccaatgggatccgcgtcaagtggagcaaggcaggtgg

SEQ ID NO: 532 - DNA, 10 bp, synthetic construct
  source 1..10: mol_type = other DNA
gctgtggcag

SEQ ID NO: 533 - DNA, 291 bp, synthetic construct
  source 1..291: mol_type = other DNA
gataagcttgatatcgcctgcagccgaattctcagcctgcatttctgccaggggaattccccggacaagccttgatgactcatctagtcagtttcggtttccgtttcccaattgacctgcagtgacctctgaaagaaagtgaaagtgacggatcaacttctagagggtatataatgggggccagaacacatcgctaagcgaaagctaagctccagtacaggggcttttgtggaacgcaaagaggaaaggtgaatccaatgggatccgcgtcaagtggagcaaggcaggtgg

SEQ ID NO: 534 - DNA, 10 bp, synthetic construct
  source 1..10: mol_type = other DNA
tggaacgcaa

SEQ ID NO: 535 - DNA, 288 bp, synthetic construct
  source 1..288: mol_type = other DNA
gataagcttgatatcgcctgcagccgaattctcagcctgcatttctgccaggggacacctgtttgcggctataaacagctgttagtcgggtttaccagctgcttctcgtctgaaagtgaaagggataacatggaagagaaagtgacggatcaacttctagagggtatataatgggggccagaacacatcgctaagcgaaagctaagctccagtacaggggcttttgctccacttgcagaggaaaggtgaatccaatgggatccgcgtcaagtggagcaaggcaggtgg

SEQ ID NO: 536 - DNA, 10 bp, synthetic construct
  source 1..10: mol_type = other DNA
ctccacttgc

SEQ ID NO: 537 - DNA, 286 bp, synthetic construct
  source 1..286: mol_type = other DNA
gataagcttgatatcgcctgcagccgaattctcagcctgcatttctgccagggcacttccgccttagagggaaacggaaaccgaaactgacaaggggaatccccttcatgtctgcacctgctcccaaggcgaggtgacgtgatcggatcaacttctagagggtatataatgggggccagaacacatcgctaagcgaaagctaagctccagtacaggggcttttgagaggaagtgagaggaaaggtgaatccaatgggatccgcgtcaagtggagcaaggcaggtgg

SEQ ID NO: 538 - DNA, 10 bp, synthetic construct
  source 1..10: mol_type = other DNA
agaggaagtg

SEQ ID NO: 539 - DNA, 287 bp, synthetic construct
  source 1..287: mol_type = other DNA
gataagcttgatatcgcctgcagccgaattctcagcctgcatttctgccagggaggggattccccttggcggcttttcctggaaatacgttagggagcaggtgcagagcgaagtttcggtttcgtttcgttataggcggaagtgcggatcaacttctagagggtatataatgggggccagaacacatcgctaagcgaaagctaagctccagtacaggggcttttgtcgcccgaccagaggaaaggtgaatccaatgggatccgcgtcaagtggagcaaggcaggtgg

SEQ ID NO: 540 - DNA, 10 bp, synthetic construct
  source 1..10: mol_type = other DNA
tcgcccgacc

SEQ ID NO: 541 - DNA, 293 bp, synthetic construct
  source 1..293: mol_type = other DNA
gataagcttgatatcgcctgcagccgaattctcagcctgcatttctgccagggatcacgtcacctatcactctcacttcctgctcaaggctagatgactcatcatttcagaggtcactgcaggtcataacagtttcggtttccgtttccccggatcaacttctagagggtatataatgggggccagaacacatcgctaagcgaaagctaagctccagtacaggggcttttgcgaatcgtcgagaggaaaggtgaatccaatgggatccgcgtcaagtggagcaaggcaggtgg

SEQ ID NO: 542 - DNA, 10 bp, synthetic construct
  source 1..10: mol_type = other DNA
cgaatcgtcg

SEQ ID NO: 543 - DNA, 288 bp, synthetic construct
  source 1..288: mol_type = other DNA
gataagcttgatatcgcctgcagccgaattctcagcctgcatttctgccagggtttccaggaaaattcttaaagatgactcatctagcgattcagtttcattttcctgcgctaggtgacgtgatttaaagctaggggaatcccctcggatcaacttctagagggtatataatgggggccagaacacatcgctaagcgaaagctaagctccagtacaggggcttttgcgagcttgacagaggaaaggtgaatccaatgggatccgcgtcaagtggagcaaggcaggtgg

SEQ ID NO: 544 - DNA, 10 bp, synthetic construct
  source 1..10: mol_type = other DNA
cgagcttgac

SEQ ID NO: 545 - DNA, 286 bp, synthetic construct
  source 1..286: mol_type = other DNA
gataagcttgatatcgcctgcagccgaattctcagcctgcatttctgccagggaacagctgttatagcgcaatgcgtgggcgtattagggaaacggaaaccgaaactgatctccggggaattcaagtgcaagattgtgcaatacggatcaacttctagagggtatataatgggggccagaacacatcgctaagcgaaagctaagctccagtacaggggcttttgcacgttgggcagaggaaaggtgaatccaatgggatccgcgtcaagtggagcaaggcaggtgg

SEQ ID NO: 546 - DNA, 10 bp, synthetic construct
  source 1..10: mol_type = other DNA
cacgttgggc

SEQ ID NO: 547 - DNA, 293 bp, synthetic construct
  source 1..293: mol_type = other DNA
gataagcttgatatcgcctgcagccgaattctcagcctgcatttctgccagggggaaaatgaaactgatgaatttaacagctgtttgtacattgaaagtgaaagtgaaaaactagaattccccggattagggaaacggaaaccgaaactgcggatcaacttctagagggtatataatgggggccagaacacatcgctaagcgaaagctaagctccagtacaggggcttttggtgaccggtcagaggaaaggtgaatccaatgggatccgcgtcaagtggagcaaggcaggtgg

SEQ ID NO: 548 - DNA, 10 bp, synthetic construct
  source 1..10: mol_type = other DNA
gtgaccggtc

SEQ ID NO: 549 - DNA, 290 bp, synthetic construct
  source 1..290: mol_type = other DNA
gataagcttgatatcgcctgcagccgaattctcagcctgcatttctgccagggctgcacctgctcccacctatctccacctgccgcctccgttctgcacctgccctcagcataacgaaaccgaaactttccaatcgaaagcgaaacacggatcaacttctagagggtatataatgggggccagaacacatcgctaagcgaaagctaagctccagtacaggggcttttgcgtccgtttcagaggaaaggtgaatccaatgggatccgcgtcaagtggagcaaggcaggtgg

SEQ ID NO: 550 - DNA, 10 bp, synthetic construct
  source 1..10: mol_type = other DNA
cgtccgtttc

SEQ ID NO: 551 - DNA, 288 bp, synthetic construct
  source 1..288: mol_type = other DNA
gataagcttgatatcgcctgcagccgaattctcagcctgcatttctgccagggctgcacctgccctcatgtcaccgcacctgccgccatcccactccacctgccgccacggcattttctaggaagagtcctcgacttccgggaagcggatcaacttctagagggtatataatgggggccagaacacatcgctaagcgaaagctaagctccagtacaggggcttttgagggatccacagaggaaaggtgaatccaatgggatccgcgtcaagtggagcaaggcaggtgg

SEQ ID NO: 552 - DNA, 10 bp, synthetic construct
  source 1..10: mol_type = other DNA
agggatccac

SEQ ID NO: 553 - DNA, 290 bp, synthetic construct
  source 1..290: mol_type = other DNA
gataagcttgatatcgcctgcagccgaattctcagcctgcatttctgccagggggaaaatgaaactgaatttcagtttcattatgactcacagctgtcacgtgacagagagtttcactttcactttcattgctctgcacctgctccccggatcaacttctagagggtatataatgggggccagaacacatcgctaagcgaaagctaagctccagtacaggggcttttgttggcccgctagaggaaaggtgaatccaatgggatccgcgtcaagtggagcaaggcaggtgg

SEQ ID NO: 554 - DNA, 10 bp, synthetic construct
  source 1..10: mol_type = other DNA
ttggcccgct

SEQ ID NO: 555 - DNA, 289 bp, synthetic construct
  source 1..289: mol_type = other DNA
gataagcttgatatcgcctgcagccgaattctcagcctgcatttctgccagggtgcgtgggcgttatcggagggagcaggtgcagttgtgaaaagatcaaaggaataaacgtgatgactcatcatattgacctgcagtgacctctgcggatcaacttctagagggtatataatgggggccagaacacatcgctaagcgaaagctaagctccagtacaggggcttttgaacggcgttgagaggaaaggtgaatccaatgggatccgcgtcaagtggagcaaggcaggtgg

SEQ ID NO: 556 - DNA, 10 bp, synthetic construct
  source 1..10: mol_type = other DNA
aacggcgttg

SEQ ID NO: 557 - DNA, 293 bp, synthetic construct
  source 1..293: mol_type = other DNA
gataagcttgatatcgcctgcagccgaattctcagcctgcatttctgccaggggggagcaggtgcagtgtccctcacttccgccttccgatatcagtttcattttccagttctaggggaatcccctagagggaaacggaaaccgaaactgcggatcaacttctagagggtatataatgggggccagaacacatcgctaagcgaaagctaagctccagtacaggggcttttgaacggacctcagaggaaaggtgaatccaatgggatccgcgtcaagtggagcaaggcaggtgg

SEQ ID NO: 558 - DNA, 10 bp, synthetic construct
  source 1..10: mol_type = other DNA
aacggacctc

SEQ ID NO: 559 - DNA, 292 bp, synthetic construct
  source 1..292: mol_type = other DNA
gataagcttgatatcgcctgcagccgaattctcagcctgcatttctgccagggagtttcggtttcgttttcagtttcggtttccgtttcccaagttgtcacgtgacaacgctactaggcggaagtgtatcttgagtcataatgaaactgcggatcaacttctagagggtatataatgggggccagaacacatcgctaagcgaaagctaagctccagtacaggggcttttgagacactacgagaggaaaggtgaatccaatgggatccgcgtcaagtggagcaaggcaggtgg

SEQ ID NO: 560 - DNA, 10 bp, synthetic construct
  source 1..10: mol_type = other DNA
agacactacg

SEQ ID NO: 561 - DNA, 290 bp, synthetic construct
  source 1..290: mol_type = other DNA
gataagcttgatatcgcctgcagccgaattctcagcctgcatttctgccagggaggcggaagtgttgtatcagtttcattatgactcaccgttattgcacaatcacgacatctcacttcctgctcagttgacctgcagtgacctctgcggatcaacttctagagggtatataatgggggccagaacacatcgctaagcgaaagctaagctccagtacaggggcttttggccggcgcgcagaggaaaggtgaatccaatgggatccgcgtcaagtggagcaaggcaggtgg

SEQ ID NO: 562 - DNA, 10 bp, synthetic construct
  source 1..10: mol_type = other DNA
gccggcgcgc

SEQ ID NO: 563 - DNA, 285 bp, synthetic construct
  source 1..285: mol_type = other DNA
gataagcttgatatcgcctgcagccgaattctcagcctgcatttctgccagggacgcccacgcaagtctcatcactttcactttctacccagagcaggaagtgagtacgtatccggggaattcagtaaacgagtcacgtgaccggatcaacttctagagggtatataatgggggccagaacacatcgctaagcgaaagctaagctccagtacaggggcttttgtcgcggccaaagaggaaaggtgaatccaatgggatccgcgtcaagtggagcaaggcaggtgg

SEQ ID NO: 564 - DNA, 10 bp, synthetic construct
  source 1..10: mol_type = other DNA
tcgcggccaa

SEQ ID NO: 565 - DNA, 289 bp, synthetic construct
  source 1..289: mol_type = other DNA
gataagcttgatatcgcctgcagccgaattctcagcctgcatttctgccaggggtcacgtgacatagtactaggggaatcccctaaagtctatgcgtgggcgtagtcgatattttcctggaaatcgtcagaggtcactgcaggtcacggatcaacttctagagggtatataatgggggccagaacacatcgctaagcgaaagctaagctccagtacaggggcttttgctgttctccgagaggaaaggtgaatccaatgggatccgcgtcaagtggagcaaggcaggtgg

SEQ ID NO: 566 - DNA, 10 bp, synthetic construct
  source 1..10: mol_type = other DNA
ctgttctccg

SEQ ID NO: 567 - DNA, 288 bp, synthetic construct
  source 1..288: mol_type = other DNA
gataagcttgatatcgcctgcagccgaattctcagcctgcatttctgccaggggtcacgtgacaccaacagtttcggtttccgtttcccaataggggaatcccctagaacgctgatgagtcatcaaaatctctcacttcctgctccggatcaacttctagagggtatataatgggggccagaacacatcgctaagcgaaagctaagctccagtacaggggcttttggccctgggagagaggaaaggtgaatccaatgggatccgcgtcaagtggagcaaggcaggtgg

SEQ ID NO: 568 - DNA, 10 bp, synthetic construct
  source 1..10: mol_type = other DNA
gccctgggag

SEQ ID NO: 569 - DNA, 296 bp, synthetic construct
  source 1..296: mol_type = other DNA
gataagcttgatatcgcctgcagccgaattctcagcctgcatttctgccagggccgcacctgccgccttgccactccacctgccgccaaacaaaggtcacctcaggccaactctgagggcactaaaggtcaatacagaggtcactgcaggtcacggatcaacttctagagggtatataatgggggccagaacacatcgctaagcgaaagctaagctccagtacaggggcttttgtagccagggaagaggaaaggtgaatccaatgggatccgcgtcaagtggagcaaggcaggtgg

SEQ ID NO: 570 - DNA, 10 bp, synthetic construct
  source 1..10: mol_type = other DNA
tagccaggga

SEQ ID NO: 571 - DNA, 286 bp, synthetic construct
  source 1..286: mol_type = other DNA
gataagcttgatatcgcctgcagccgaattctcagcctgcatttctgccaggggtcacgtgcctccagcagtctgagtcagcatgattagcaaagtaaacaaatatgtagagtcataatgaaactgaatctaaaagataagaacggatcaacttctagagggtatataatgggggccagaacacatcgctaagcgaaagctaagctccagtacaggggcttttgtgtcacttgcagaggaaaggtgaatccaatgggatccgcgtcaagtggagcaaggcaggtgg

SEQ ID NO: 572 - DNA, 10 bp, synthetic construct
  source 1..10: mol_type = other DNA
tgtcacttgc

SEQ ID NO: 573 - DNA, 287 bp, synthetic construct
  source 1..287: mol_type = other DNA
gataagcttgatatcgcctgcagccgaattctcagcctgcatttctgccagggcagtttcattatgactcagaaacaatgtgcagtgtttgcagatgtcacgtgcctcctcgttagtttcggtttcggtcgcttgtaaatgactcggatcaacttctagagggtatataatgggggccagaacacatcgctaagcgaaagctaagctccagtacaggggcttttgcatactgcgtagaggaaaggtgaatccaatgggatccgcgtcaagtggagcaaggcaggtgg

SEQ ID NO: 574 - DNA, 10 bp, synthetic construct
  source 1..10: mol_type = other DNA
catactgcgt

SEQ ID NO: 575 - DNA, 288 bp, synthetic construct
  source 1..288: mol_type = other DNA
gataagcttgatatcgcctgcagccgaattctcagcctgcatttctgccagggtagtttcggtttcggtacagagtcataatgaaactgagacaaaaggtgtgaaaaccattcgcatagtcatttaccctaagcaggcacgtgaccggatcaacttctagagggtatataatgggggccagaacacatcgctaagcgaaagctaagctccagtacaggggcttttggcacatcttcagaggaaaggtgaatccaatgggatccgcgtcaagtggagcaaggcaggtgg

SEQ ID NO: 576 - DNA, 10 bp, synthetic construct
  source 1..10: mol_type = other DNA
gcacatcttc

SEQ ID NO: 577 - DNA, 286 bp, synthetic construct
  source 1..286: mol_type = other DNA
gataagcttgatatcgcctgcagccgaattctcagcctgcatttctgccagggaagtaaacaaattgaaagtccacttccggtctaataccttgtcacgtgcctcgtcaataaaataaacaatttacctacatatcaggttaccggatcaacttctagagggtatataatgggggccagaacacatcgctaagcgaaagctaagctccagtacaggggcttttgacgtccgcttagaggaaaggtgaatccaatgggatccgcgtcaagtggagcaaggcaggtgg

SEQ ID NO: 578 - DNA, 10 bp, synthetic construct
  source 1..10: mol_type = other DNA
acgtccgctt

SEQ ID NO: 579 - DNA, 285 bp, synthetic construct
  source 1..285: mol_type = other DNA
gataagcttgatatcgcctgcagccgaattctcagcctgcatttctgccagggggcacgtgacatagcgactcaagtcatttcgtctgtgaatgcacctgttatactactccacttccggtcttatgtaaattcttatctttcggatcaacttctagagggtatataatgggggccagaacacatcgctaagcgaaagctaagctccagtacaggggcttttggcgccggagcagaggaaaggtgaatccaatgggatccgcgtcaagtggagcaaggcaggtgg

SEQ ID NO: 580 - DNA, 286 bp, synthetic construct
  source 1..286: mol_type = other DNA
gataagcttgatatcgcctgcagccgaattctcagcctgcatttctgccagggccacttccggtcttgtccgtaaacaggtgcaagtaactaagtaacctgataatacgcgtactaatgacttatgtccctgtggcacgtgaccggatcaacttctagagggtatataatgggggccagaacacatcgctaagcgaaagctaagctccagtacaggggcttttgtcagcgtggaagaggaaaggtgaatccaatgggatccgcgtcaagtggagcaaggcaggtgg

SEQ ID NO: 581 - DNA, 10 bp, synthetic construct
  source 1..10: mol_type = other DNA
tcagcgtgga

SEQ ID NO: 582 - DNA, 285 bp, synthetic construct
  source 1..285: mol_type = other DNA
gataagcttgatatcgcctgcagccgaattctcagcctgcatttctgccaggggtaacctgatatatccattatgctgactcagatctcaaacaatgtgcagtgtttatctccacttccggtctactgacaagtcacgtgcccggatcaacttctagagggtatataatgggggccagaacacatcgctaagcgaaagctaagctccagtacaggggcttttgccgcatatccagaggaaaggtgaatccaatgggatccgcgtcaagtggagcaaggcaggtgg

SEQ ID NO: 583 - DNA, 10 bp, synthetic construct
  source 1..10: mol_type = other DNA
ccgcatatcc

SEQ ID NO: 584 - DNA, 288 bp, synthetic construct
  source 1..288: mol_type = other DNA
gataagcttgatatcgcctgcagccgaattctcagcctgcatttctgccagggccgaaaccgaaactaatagcttatatttacataatcgatgacaaaatgacttcctcgaattaaataaacaatttagcatcctgtcacgtgcccggatcaacttctagagggtatataatgggggccagaacacatcgctaagcgaaagctaagctccagtacaggggcttttgccacgttgccagaggaaaggtgaatccaatgggatccgcgtcaagtggagcaaggcaggtgg

SEQ ID NO: 585 - DNA, 10 bp, synthetic construct
  source 1..10: mol_type = other DNA
ccacgttgcc

SEQ ID NO: 586 - DNA, 286 bp, synthetic construct
  source 1..286: mol_type = other DNA
gataagcttgatatcgcctgcagccgaattctcagcctgcatttctgccaggggtaacctgatatccccgaatggcacgtgacttcactaccgaaaccgaaactaaacgtctttcttatctttactttatcatttgtttacttcggatcaacttctagagggtatataatgggggccagaacacatcgctaagcgaaagctaagctccagtacaggggcttttgtgtgcagactagaggaaaggtgaatccaatgggatccgcgtcaagtggagcaaggcaggtgg

SEQ ID NO: 587 - DNA, 10 bp, synthetic construct
  source 1..10: mol_type = other DNA
tgtgcagact

SEQ ID NO: 588 - DNA, 285 bp, synthetic construct
  source 1..285: mol_type = other DNA
gataagcttgatatcgcctgcagccgaattctcagcctgcatttctgccagggcaaaggtcagactcgatcaaaataaacaatttaagttatagtcacgtgcctactttatagtttcggtttcggatgtgacagctaatggacggatcaacttctagagggtatataatgggggccagaacacatcgctaagcgaaagctaagctccagtacaggggcttttgcgacgtccagagaggaaaggtgaatccaatgggatccgcgtcaagtggagcaaggcaggtgg

SEQ ID NO: 589 - DNA, 10 bp, synthetic construct
  source 1..10: mol_type = other DNA
cgacgtccag

SEQ ID NO: 590 - DNA, 284 bp, synthetic construct
  source 1..284: mol_type = other DNA
gataagcttgatatcgcctgcagccgaattctcagcctgcatttctgccagggttttccatcagtgcgtagcatgactcagcattcgtagacaagtcatttatactaataaaataaacaattagacttgttgtcacgtgcccggatcaacttctagagggtatataatgggggccagaacacatcgctaagcgaaagctaagctccagtacaggggcttttggacgttggttagaggaaaggtgaatccaatgggatccgcgtcaagtggagcaaggcaggtgg

SEQ ID NO: 591 - DNA, 10 bp, synthetic construct
  source 1..10: mol_type = other DNA
gacgttggtt

SEQ ID NO: 592 - DNA, 284 bp, synthetic construct
  source 1..284: mol_type = other DNA
gataagcttgatatcgcctgcagccgaattctcagcctgcatttctgccagggtgcacctgtttcattccatgctgagtcatgcaggaccctggcacgtgacattttataacactgcacattgttagtggctgctaatggacggatcaacttctagagggtatataatgggggccagaacacatcgctaagcgaaagctaagctccagtacaggggcttttgcctgcccaggagaggaaaggtgaatccaatgggatccgcgtcaagtggagcaaggcaggtgg

SEQ ID NO: 593 - DNA, 10 bp, synthetic construct
  source 1..10: mol_type = other DNA
cctgcccagg

SEQ ID NO: 594 - DNA, 286 bp, synthetic construct
  source 1..286: mol_type = other DNA
gataagcttgatatcgcctgcagccgaattctcagcctgcatttctgccagggatatttacataatactagcgcttggaaaaacacgttcatagaccggaagtggtcttacagtttcattatgactcttggcacaaaggtcagcggatcaacttctagagggtatataatgggggccagaacacatcgctaagcgaaagctaagctccagtacaggggcttttggaatgggcctagaggaaaggtgaatccaatgggatccgcgtcaagtggagcaaggcaggtgg

SEQ ID NO: 595 - DNA, 10 bp, synthetic construct
  source 1..10: mol_type = other DNA
gaatgggcct

SEQ ID NO: 596 - DNA, 290 bp, synthetic construct
  source 1..290: mol_type = other DNA
gataagcttgatatcgcctgcagccgaattctcagcctgcatttctgccagggtttgtttactttttaaacattgctgactcagacagtgacaggaagcagatgtgagtcgtagaccggaagtggtaactgagtcataatgaaactgcggatcaacttctagagggtatataatgggggccagaacacatcgctaagcgaaagctaagctccagtacaggggcttttgaggagctatgagaggaaaggtgaatccaatgggatccgcgtcaagtggagcaaggcaggtgg

SEQ ID NO: 597 - DNA, 10 bp, synthetic construct
  source 1..10: mol_type = other DNA
aggagctatg

SEQ ID NO: 598 - DNA, 291 bp, synthetic construct
  source 1..291: mol_type = other DNA
gataagcttgatatcgcctgcagccgaattctcagcctgcatttctgccagggcacatctgcttcctgtagagtccacttccggtctagtacgaacaaaggtcagtgtagtttagtttcggtttcggacttcagtttcattatgactccggatcaacttctagagggtatataatgggggccagaacacatcgctaagcgaaagctaagctccagtacaggggcttttgcggccagccgagaggaaaggtgaatccaatgggatccgcgtcaagtggagcaaggcaggtgg

SEQ ID NO: 599 - DNA, 10 bp, synthetic construct
  source 1..10: mol_type = other DNA
cggccagccg

SEQ ID NO: 600 - DNA, 290 bp, synthetic construct
  source 1..290: mol_type = other DNA
gataagcttgatatcgcctgcagccgaattctcagcctgcatttctgccaggggagtcataatgaaactgaagtccgaaaccgaaactaacataagcatgactcagcatcggggtatatcaggttactcttcgcgtatatttacatacggatcaacttctagagggtatataatgggggccagaacacatcgctaagcgaaagctaagctccagtacaggggcttttggctcaaacgaagaggaaaggtgaatccaatgggatccgcgtcaagtggagcaaggcaggtgg

SEQ ID NO: 601 - DNA, 10 bp, synthetic construct
  source 1..10: mol_type = other DNA
gctcaaacga

SEQ ID NO: 602 - DNA, 289 bp, synthetic construct
  source 1..289: mol_type = other DNA
gataagcttgatatcgcctgcagccgaattctcagcctgcatttctgccagggcagtttcattatgactctcgctaaaggtgtgaaatcccgattttatgtaaatattaattctctgtagtcattatagcaataagttccattagccggatcaacttctagagggtatataatgggggccagaacacatcgctaagcgaaagctaagctccagtacaggggcttttgtgctcatgctagaggaaaggtgaatccaatgggatccgcgtcaagtggagcaaggcaggtgg

SEQ ID NO: 603 - DNA, 10 bp, synthetic construct
  source 1..10: mol_type = other DNA
tgctcatgct

SEQ ID NO: 604 - DNA, 284 bp, synthetic construct
  source 1..284: mol_type = other DNA
gataagcttgatatcgcctgcagccgaattctcagcctgcatttctgccagggttttccaaagccgatcagtttcattatgactcaacacaggaagcagatgtgtcatagaaaagataagaaacaacatttaacaggtgcacggatcaacttctagagggtatataatgggggccagaacacatcgctaagcgaaagctaagctccagtacaggggcttttgcgccgtcggcagaggaaaggtgaatccaatgggatccgcgtcaagtggagcaaggcaggtgg

SEQ ID NO: 605 - DNA, 10 bp, synthetic construct
  source 1..10: mol_type = other DNA
cgccgtcggc

SEQ ID NO: 606 - DNA, 292 bp, synthetic construct
  source 1..292: mol_type = other DNA
gataagcttgatatcgcctgcagccgaattctcagcctgcatttctgccagggaacactgcacattgtttaaagaaaagataagaaaccgagtcaggaagcagatgtgactaatagtaacctgatattgaaacagtttcattatgactccggatcaacttctagagggtatataatgggggccagaacacatcgctaagcgaaagctaagctccagtacaggggcttttgctagtctccgagaggaaaggtgaatccaatgggatccgcgtcaagtggagcaaggcaggtgg

SEQ ID NO: 607 - DNA, 10 bp, synthetic construct
  source 1..10: mol_type = other DNA
ctagtctccg

SEQ ID NO: 608 - DNA, 290 bp, synthetic construct
  source 1..290: mol_type = other DNA
gataagcttgatatcgcctgcagccgaattctcagcctgcatttctgccagggtttcacaccttttgggttaactgagtcagcattacgactcaaaatgacttcaccagattgcatgactcagcaaatgtgagtcataatgaaactgcggatcaacttctagagggtatataatgggggccagaacacatcgctaagcgaaagctaagctccagtacaggggcttttgctgtggcgccagaggaaaggtgaatccaatgggatccgcgtcaagtggagcaaggcaggtgg

SEQ ID NO: 609 - DNA, 10 bp, synthetic construct
  source 1..10: mol_type = other DNA
ctgtggcgcc

SEQ ID NO: 610 - DNA, 287 bp, synthetic construct
  source 1..287: mol_type = other DNA
gataagcttgatatcgcctgcagccgaattctcagcctgcatttctgccagggaacactgcacattgtttctcagtttcattatgactctggtgtgtaacctgatatggtgttcacatctgcttcctgaatcatataaatgactcggatcaacttctagagggtatataatgggggccagaacacatcgctaagcgaaagctaagctccagtacaggggcttttggtgggtgttgagaggaaaggtgaatccaatgggatccgcgtcaagtggagcaaggcaggtgg

SEQ ID NO: 611 - DNA, 10 bp, synthetic construct
  source 1..10: mol_type = other DNA
gtgggtgttg

SEQ ID NO: 612 - DNA, 286 bp, synthetic construct
  source 1..286: mol_type = other DNA
gataagcttgatatcgcctgcagccgaattctcagcctgcatttctgccagggccacttccggtcttttgacaaattgtttatttttttagagatgcacctgttacactagaagtaacctgataaagaacatacaaaggtcagcggatcaacttctagagggtatataatgggggccagaacacatcgctaagcgaaagctaagctccagtacaggggcttttgttgcagagggagaggaaaggtgaatccaatgggatccgcgtcaagtggagcaaggcaggtgg

SEQ ID NO: 613 - DNA, 10 bp, synthetic construct
  source 1..10: mol_type = other DNA
ttgcagaggg

SEQ ID NO: 614 - DNA, 286 bp, synthetic construct
  source 1..286: mol_type = other DNA
gataagcttgatatcgcctgcagccgaattctcagcctgcatttctgccagggtccattagcaggcctaataattgtttatttatagatctactgacctttgaatagcgcctaaatgacttcgttgcgatccacttccggtctcggatcaacttctagagggtatataatgggggccagaacacatcgctaagcgaaagctaagctccagtacaggggcttttgacgttgtacgagaggaaaggtgaatccaatgggatccgcgtcaagtggagcaaggcaggtgg

SEQ ID NO: 615 - DNA, 10 bp, synthetic construct
  source 1..10: mol_type = other DNA
acgttgtacg

SEQ ID NO: 616 - DNA, 289 bp, synthetic construct
  source 1..289: mol_type = other DNA
gataagcttgatatcgcctgcagccgaattctcagcctgcatttctgccagggagaccggaagtggacgtaacacatctgcttcctgtagttaaaataaacaattaaagtcgagtaacctgatatcgtgtaacaatgtgcagtgttcggatcaacttctagagggtatataatgggggccagaacacatcgctaagcgaaagctaagctccagtacaggggcttttgtgcagccggaagaggaaaggtgaatccaatgggatccgcgtcaagtggagcaaggcaggtgg

SEQ ID NO: 617 - DNA, 10 bp, synthetic construct
  source 1..10: mol_type = other DNA
tgcagccgga

SEQ ID NO: 618 - DNA, 289 bp, synthetic construct
  source 1..289: mol_type = other DNA
gataagcttgatatcgcctgcagccgaattctcagcctgcatttctgccagggagaccggaagtggagccgtcttgcacctgttatccgtgtatatcaggttacttcagtgaactttttccataacgttaaacaatgtgcagtgttcggatcaacttctagagggtatataatgggggccagaacacatcgctaagcgaaagctaagctccagtacaggggcttttggtcgcatgctagaggaaaggtgaatccaatgggatccgcgtcaagtggagcaaggcaggtgg

SEQ ID NO: 619 - DNA, 10 bp, synthetic construct
  source 1..10: mol_type = other DNA
gtcgcatgct

SEQ ID NO: 620 - DNA, 288 bp, synthetic construct
  source 1..288: mol_type = other DNA
gataagcttgatatcgcctgcagccgaattctcagcctgcatttctgccaggggcatgactcagcatactgttttatcaggttacaagccgaatgtaatgactacatttaataagaccggaagtggagcttacttttcacaccttcggatcaacttctagagggtatataatgggggccagaacacatcgctaagcgaaagctaagctccagtacaggggcttttgcctaagtaggagaggaaaggtgaatccaatgggatccgcgtcaagtggagcaaggcaggtgg

SEQ ID NO: 621 - DNA, 10 bp, synthetic construct
  source 1..10: mol_type = other DNA
cctaagtagg

SEQ ID NO: 622 - DNA, 285 bp, synthetic construct
  source 1..285: mol_type = other DNA
gataagcttgatatcgcctgcagccgaattctcagcctgcatttctgccagggaatgactacacaactacgagctaatggaagcaacccgttgctgactcagtagtacaaaaagtaaacaaaagtcgcctttatgtaaatatcggatcaacttctagagggtatataatgggggccagaacacatcgctaagcgaaagctaagctccagtacaggggcttttggccaacacgaagaggaaaggtgaatccaatgggatccgcgtcaagtggagcaaggcaggtgg

SEQ ID NO: 623 - DNA, 286 bp, synthetic construct
  source 1..286: mol_type = other DNA
gataagcttgatatcgcctgcagccgaattctcagcctgcatttctgccagggtttgtttacttttcctattaattgtttatttaagtagcataacaggtgcatgccctaccgaaaccgaaactatgcatttaaagataagaacggatcaacttctagagggtatataatgggggccagaacacatcgctaagcgaaagctaagctccagtacaggggcttttgacaacggagcagaggaaaggtgaatccaatgggatccgcgtcaagtggagcaaggcaggtgg

SEQ ID NO: 624 - DNA, 285 bp, synthetic construct
  source 1..285: mol_type = other DNA
gataagcttgatatcgcctgcagccgaattctcagcctgcatttctgccagggaaagataagaaacctcttaaaagtaaacaaaaagtatagttttcacacctttatattctaaataaacaattatggtacaatccattagccggatcaacttctagagggtatataatgggggccagaacacatcgctaagcgaaagctaagctccagtacaggggcttttgcgctggtaaaagaggaaaggtgaatccaatgggatccgcgtcaagtggagcaaggcaggtgg

SEQ ID NO: 625 - DNA, 10 bp, synthetic construct
  source 1..10: mol_type = other DNA
cgctggtaaa

SEQ ID NO: 626 - DNA, 291 bp, synthetic construct
  source 1..291: mol_type = other DNA
gataagcttgatatcgcctgcagccgaattctcagcctgcatttctgccagggccgaaaccgaaactatgctctaaaggtgtgaaaatcgacgatgctgagtcatgcactatattaagtaaacaaaacttaatcaggaagcagatgtgcggatcaacttctagagggtatataatgggggccagaacacatcgctaagcgaaagctaagctccagtacaggggcttttggaaacgccgcagaggaaaggtgaatccaatgggatccgcgtcaagtggagcaaggcaggtgg

SEQ ID NO: 627 - DNA, 286 bp, synthetic construct
  source 1..286: mol_type = other DNA
gataagcttgatatcgcctgcagccgaattctcagcctgcatttctgccagggtttgtttacttaccaaatcagtaacctgatatacgacatacaaaggtcagtgtcactgcgtttttccaaccgtaagttactgagtcagcacggatcaacttctagagggtatataatgggggccagaacacatcgctaagcgaaagctaagctccagtacaggggcttttgctatcgctacagaggaaaggtgaatccaatgggatccgcgtcaagtggagcaaggcaggtgg

SEQ ID NO: 628 - DNA, 10 bp, synthetic construct
  source 1..10: mol_type = other DNA
ctatcgctac

SEQ ID NO: 629 - DNA, 284 bp, synthetic construct
  source 1..284: mol_type = other DNA
gataagcttgatatcgcctgcagccgaattctcagcctgcatttctgccagggagtcatttaggtccttcgattggaaaaatcacaagggttgctgactcagtatagcgaaaataaacaattaattcacaaaagataagaacggatcaacttctagagggtatataatgggggccagaacacatcgctaagcgaaagctaagctccagtacaggggcttttgcgtcgcgcatagaggaaaggtgaatccaatgggatccgcgtcaagtggagcaaggcaggtgg

SEQ ID NO: 630 - DNA, 10 bp, synthetic construct
  source 1..10: mol_type = other DNA
cgtcgcgcat

SEQ ID NO: 631 - DNA, 288 bp, synthetic construct
  source 1..288: mol_type = other DNA
gataagcttgatatcgcctgcagccgaattctcagcctgcatttctgccagggctgagtcagcaatctgtgaaattgtttattttcgtacacttttttccaatcttagttccgaaaccgaaactaaagcacaggaagcagatgtgcggatcaacttctagagggtatataatgggggccagaacacatcgctaagcgaaagctaagctccagtacaggggcttttgaccttcgccaagaggaaaggtgaatccaatgggatccgcgtcaagtggagcaaggcaggtgg

SEQ ID NO: 632 - DNA, 10 bp, synthetic construct
  source 1..10: mol_type = other DNA
accttcgcca

SEQ ID NO: 633 - DNA, 289 bp, synthetic construct
  source 1..289: mol_type = other DNA
gataagcttgatatcgcctgcagccgaattctcagcctgcatttctgccagggaacaatgtgcagtgttttggttgctgagtcatgcacgcattagtttcggtttcggtgagactaacaggtgcatcacgaaataattgtttatttcggatcaacttctagagggtatataatgggggccagaacacatcgctaagcgaaagctaagctccagtacaggggcttttgcgcttcttccagaggaaaggtgaatccaatgggatccgcgtcaagtggagcaaggcaggtgg

SEQ ID NO: 634 - DNA, 10 bp, synthetic construct
  source 1..10: mol_type = other DNA
cgcttcttcc

SEQ ID NO: 635 - DNA, 287 bp, synthetic construct
  source 1..287: mol_type = other DNA
gataagcttgatatcgcctgcagccgaattctcagcctgcatttctgccagggctgagtcagcaactatggtgaattttccaaagtacatcactgctaatggaaacctgtattctgacctttgatagtgcataaataaacaattcggatcaacttctagagggtatataatgggggccagaacacatcgctaagcgaaagctaagctccagtacaggggcttttggccgcgagtcagaggaaaggtgaatccaatgggatccgcgtcaagtggagcaaggcaggtgg

SEQ ID NO: 636 - DNA, 10 bp, synthetic construct
  source 1..10: mol_type = other DNA
gccgcgagtc

SEQ ID NO: 637 - DNA, 286 bp, synthetic construct
  source 1..286: mol_type = other DNA
gataagcttgatatcgcctgcagccgaattctcagcctgcatttctgccagggtatcaggttacaagactctaaaggtgtgaaaacccaagttgctgagtcatgctgtcgtaaaataaacaatttctgtagatctgacctttgcggatcaacttctagagggtatataatgggggccagaacacatcgctaagcgaaagctaagctccagtacaggggcttttgattgaaccggagaggaaaggtgaatccaatgggatccgcgtcaagtggagcaaggcaggtgg

SEQ ID NO: 638 - DNA, 10 bp, synthetic construct
  source 1..10: mol_type = other DNA
attgaaccgg

SEQ ID NO: 639 - DNA, 290 bp, synthetic construct
  source 1..290: mol_type = other DNA
gataagcttgatatcgcctgcagccgaattctcagcctgcatttctgccagggaacactgcacattgtttatcctaaagataagaaagcggtcacaattttccataaacatagcgtgctaatggaatagtctatgcatgactcagcacggatcaacttctagagggtatataatgggggccagaacacatcgctaagcgaaagctaagctccagtacaggggcttttgagttccactcagaggaaaggtgaatccaatgggatccgcgtcaagtggagcaaggcaggtgg

SEQ ID NO: 640 - DNA, 10 bp, synthetic construct
  source 1..10: mol_type = other DNA
agttccactc

SEQ ID NO: 641 - DNA, 282 bp, synthetic construct
  source 1..282: mol_type = other DNA
gataagcttgatatcgcctgcagccgaattctcagcctgcatttctgccagggtggaaaaacgagtgctatgctgagtcatgctctaaataaacaggtgcataggagtccgaaaccgaaactaaacgtccgtagtcattcggatcaacttctagagggtatataatgggggccagaacacatcgctaagcgaaagctaagctccagtacaggggcttttgaccgcgtttcagaggaaaggtgaatccaatgggatccgcgtcaagtggagcaaggcaggtgg

SEQ ID NO: 642 - DNA, 10 bp, synthetic construct
  source 1..10: mol_type = other DNA
accgcgtttc

SEQ ID NO: 643 - DNA, 288 bp, synthetic construct
  source 1..288: mol_type = other DNA
gataagcttgatatcgcctgcagccgaattctcagcctgcatttctgccagggaacaatgtgcagtgtttcgaggtctgacctttgtcacaatgactagtcattatccgtctaccgaaaccgaaactaacgaaatatccattagccggatcaacttctagagggtatataatgggggccagaacacatcgctaagcgaaagctaagctccagtacaggggcttttgttgcgaagagagaggaaaggtgaatccaatgggatccgcgtcaagtggagcaaggcaggtgg

SEQ ID NO: 644 - DNA, 10 bp, synthetic construct
  source 1..10: mol_type = other DNA
ttgcgaagag

SEQ ID NO: 645 - DNA, 296 bp, synthetic construct
  source 1..296: mol_type = other DNA
gataagcttgatatcgcctgcagccgaattctcagcctgcatttctgccagggctagtttggcaacaggcatcttccacccacctgggaggcagggttcaacactctgcctctgaccttttttccttcctctgccacctgcttaggcagccagcggatcaacttctagagggtatataatgggggccagaacacatcgctaagcgaaagctaagctccagtacaggggcttttgccggatagctagaggaaaggtgaatccaatgggatccgcgtcaagtggagcaaggcaggtgg

SEQ ID NO: 646 - DNA, 10 bp, synthetic construct
  source 1..10: mol_type = other DNA
ccggatagct

SEQ ID NO: 647 - DNA, 296 bp, synthetic construct
  source 1..296: mol_type = other DNA
gataagcttgatatcgcctgcagccgaattctcagcctgcatttctgccagggagatgcacttcgcttttgttatttttttagtagagagggggtttcactggtttagccagaacagtctcgatctcctgacctcgtgatccgcccacctcggcggatcaacttctagagggtatataatgggggccagaacacatcgctaagcgaaagctaagctccagtacaggggcttttgaaatggacccagaggaaaggtgaatccaatgggatccgcgtcaagtggagcaaggcaggtgg

SEQ ID NO: 648 - DNA, 10 bp, synthetic construct
  source 1..10: mol_type = other DNA
aaatggaccc

SEQ ID NO: 649 - DNA, 288 bp, synthetic construct
  source 1..288: mol_type = other DNA
gataagcttgatatcgcctgcagccgaattctcagcctgcatttctgccaggggagtcataatgaaactgaactctctgacctttgtctctcacacatctgcttcctgaagagctggcacgtgactttatctaattcttatctttcggatcaacttctagagggtatataatgggggccagaacacatcgctaagcgaaagctaagctccagtacaggggcttttgctgttgggcgagaggaaaggtgaatccaatgggatccgcgtcaagtggagcaaggcaggtgg

SEQ ID NO: 650 - DNA, 10 bp, synthetic construct
  source 1..10: mol_type = other DNA
ctgttgggcg

SEQ ID NO: 651 - DNA, 287 bp, synthetic construct
  source 1..287: mol_type = other DNA
gataagcttgatatcgcctgcagccgaattctcagcctgcatttctgccagggtatcaggttacatggagtgtgtcacgtgccacgaagttaaaagataagaaagctttttatatggaaaatctcgactaaagaccggaagtggcggatcaacttctagagggtatataatgggggccagaacacatcgctaagcgaaagctaagctccagtacaggggcttttgacttctgctcagaggaaaggtgaatccaatgggatccgcgtcaagtggagcaaggcaggtgg

SEQ ID NO: 652 - DNA, 10 bp, synthetic construct
  source 1..10: mol_type = other DNA
acttctgctc

SEQ ID NO: 653 - DNA, 286 bp, synthetic construct
  source 1..286: mol_type = other DNA
gataagcttgatatcgcctgcagccgaattctcagcctgcatttctgccagggtatgtaaatattaaaggtattatcaggttactagtatgtaaataaacaattttctccgtaggcacgtgactcgcgacaattcttatctttcggatcaacttctagagggtatataatgggggccagaacacatcgctaagcgaaagctaagctccagtacaggggcttttgtgctctcgccagaggaaaggtgaatccaatgggatccgcgtcaagtggagcaaggcaggtgg

SEQ ID NO: 654 - DNA, 10 bp, synthetic construct
  source 1..10: mol_type = other DNA
tgctctcgcc

SEQ ID NO: 655 - DNA, 289 bp, synthetic construct
  source 1..289: mol_type = other DNA
gataagcttgatatcgcctgcagccgaattctcagcctgcatttctgccagggtagtttcggtttcggtatcgcatatgtaaatatatccatgtatgcacctgtttgcattgctagtcacgtgcctataacatgcatgactcagcacggatcaacttctagagggtatataatgggggccagaacacatcgctaagcgaaagctaagctccagtacaggggcttttgactgcccgccagaggaaaggtgaatccaatgggatccgcgtcaagtggagcaaggcaggtgg

SEQ ID NO: 656 - DNA, 10 bp, synthetic construct
  source 1..10: mol_type = other DNA
actgcccgcc

SEQ ID NO: 657 - DNA, 284 bp, synthetic construct
  source 1..284: mol_type = other DNA
gataagcttgatatcgcctgcagccgaattctcagcctgcatttctgccagggctgacctttgagggcgaataggcacgtgacttttcgttaatatttacatatggcgacttgctgagtcatgctgatgcgtgaaatgactcggatcaacttctagagggtatataatgggggccagaacacatcgctaagcgaaagctaagctccagtacaggggcttttgggccttcaaaagaggaaaggtgaatccaatgggatccgcgtcaagtggagcaaggcaggtgg

SEQ ID NO: 658 - DNA, 10 bp, synthetic construct
  source 1..10: mol_type = other DNA
ggccttcaaa

SEQ ID NO: 659 - DNA, 286 bp, synthetic construct
  source 1..286: mol_type = other DNA
gataagcttgatatcgcctgcagccgaattctcagcctgcatttctgccagggccgaaaccgaaactattcagaaagctaatggatccgaattgtgtaacctgataaacggtttagtcacgtgccacgccgaacgtaatgactcggatcaacttctagagggtatataatgggggccagaacacatcgctaagcgaaagctaagctccagtacaggggcttttgacgaggtgatagaggaaaggtgaatccaatgggatccgcgtcaagtggagcaaggcaggtgg

SEQ ID NO: 660 - DNA, 10 bp, synthetic construct
  source 1..10: mol_type = other DNA
acgaggtgat

SEQ ID NO: 661 - DNA, 286 bp, synthetic construct
  source 1..286: mol_type = other DNA
gataagcttgatatcgcctgcagccgaattctcagcctgcatttctgccaggggcatgactcagcattatcataaacaggtgcaagccactaacaatgtgcagtgtttctgtactgagtcagcaatctccggtgtcacgtgcccggatcaacttctagagggtatataatgggggccagaacacatcgctaagcgaaagctaagctccagtacaggggcttttggcgatccctgagaggaaaggtgaatccaatgggatccgcgtcaagtggagcaaggcaggtgg

SEQ ID NO: 662 - DNA, 10 bp, synthetic construct
  source 1..10: mol_type = other DNA
gcgatccctg

SEQ ID NO: 663 - DNA, 284 bp, synthetic construct
  source 1..284: mol_type = other DNA
gataagcttgatatcgcctgcagccgaattctcagcctgcatttctgccagggtggaaaatatcccgagtatgcacctgtttctggtgtccacttccggtctttctacagtttcattatgactctttgtaaagtaaacaaacggatcaacttctagagggtatataatgggggccagaacacatcgctaagcgaaagctaagctccagtacaggggcttttgtccgagtgacagaggaaaggtgaatccaatgggatccgcgtcaagtggagcaaggcaggtgg

SEQ ID NO: 664 - DNA, 10 bp, synthetic construct
  source 1..10: mol_type = other DNA
tccgagtgac

SEQ ID NO: 665 - DNA, 292 bp, synthetic construct
  source 1..292: mol_type = other DNA
gataagcttgatatcgcctgcagccgaattctcagcctgcatttctgccagggcagtttcattatgactcaacatagtttcggtttcggaggcatccacttccggtcttgttcgcattccattagcagcgtcgacaggaagcagatgtgcggatcaacttctagagggtatataatgggggccagaacacatcgctaagcgaaagctaagctccagtacaggggcttttggtgctccatgagaggaaaggtgaatccaatgggatccgcgtcaagtggagcaaggcaggtgg

SEQ ID NO: 666 - DNA, 10 bp, synthetic construct
  source 1..10: mol_type = other DNA
gtgctccatg

SEQ ID NO: 667 - DNA, 286 bp, synthetic construct
  source 1..286: mol_type = other DNA
gataagcttgatatcgcctgcagccgaattctcagcctgcatttctgccagggcagtttcattatgactctaggttaacaggtgcaatcccgatcaaagtcattagcgttctcgtcaaaggtcagtttcccgagttttttccacggatcaacttctagagggtatataatgggggccagaacacatcgctaagcgaaagctaagctccagtacaggggcttttgctgtactgcgagaggaaaggtgaatccaatgggatccgcgtcaagtggagcaaggcaggtgg

SEQ ID NO: 668 - DNA, 10 bp, synthetic construct
  source 1..10: mol_type = other DNA
ctgtactgcg

SEQ ID NO: 669 - DNA, 287 bp, synthetic construct
  source 1..287: mol_type = other DNA
gataagcttgatatcgcctgcagccgaattctcagcctgcatttctgccagggtttgtttacttattggtccttgcacctgttttgatagataaggtgtgaaatgctttgacgattttccatgaacaggtaccacttccggtctcggatcaacttctagagggtatataatgggggccagaacacatcgctaagcgaaagctaagctccagtacaggggcttttgtgtgatcgacagaggaaaggtgaatccaatgggatccgcgtcaagtggagcaaggcaggtgg

SEQ ID NO: 670 - DNA, 10 bp, synthetic construct
  source 1..10: mol_type = other DNA
tgtgatcgac

SEQ ID NO: 671 - DNA, 286 bp, synthetic construct
  source 1..286: mol_type = other DNA
gataagcttgatatcgcctgcagccgaattctcagcctgcatttctgccagggaaggtgtgaaaatcgcagtagaccggaagtggagtccaataaagataagaaattggtacaggaagcagatgtgtagttctctgacctttgcggatcaacttctagagggtatataatgggggccagaacacatcgctaagcgaaagctaagctccagtacaggggcttttgtatgcatcggagaggaaaggtgaatccaatgggatccgcgtcaagtggagcaaggcaggtgg

SEQ ID NO: 672 - DNA, 10 bp, synthetic construct
  source 1..10: mol_type = other DNA
tatgcatcgg

SEQ ID NO: 673 - DNA, 287 bp, synthetic construct
  source 1..287: mol_type = other DNA
gataagcttgatatcgcctgcagccgaattctcagcctgcatttctgccaggggctaatggataccgatattgctgagtcatgcatacgacaggaagcagatgtgtatctccgaaaccgaaactaacgggaccacttccggtctcggatcaacttctagagggtatataatgggggccagaacacatcgctaagcgaaagctaagctccagtacaggggcttttgggctccgatgagaggaaaggtgaatccaatgggatccgcgtcaagtggagcaaggcaggtgg

SEQ ID NO: 674 - DNA, 10 bp, synthetic construct
  source 1..10: mol_type = other DNA
ggctccgatg

SEQ ID NO: 675 - DNA, 288 bp, synthetic construct
  source 1..288: mol_type = other DNA
gataagcttgatatcgcctgcagccgaattctcagcctgcatttctgccagggtagtttcggtttcggtgcgtgttgctgactcagacgcaacatctgacctttgtatctgcaatatgtaaatattcagagactaagtaaacaaacggatcaacttctagagggtatataatgggggccagaacacatcgctaagcgaaagctaagctccagtacaggggcttttgaggaccctgtagaggaaaggtgaatccaatgggatccgcgtcaagtggagcaaggcaggtgg

SEQ ID NO: 676 - DNA, 10 bp, synthetic construct
  source 1..10: mol_type = other DNA
aggaccctgt

SEQ ID NO: 677 - DNA, 282 bp, synthetic construct
  source 1..282: mol_type = other DNA
gataagcttgatatcgcctgcagccgaattctcagcctgcatttctgccagggtggaaaaagctgactatatatgtaaatattcgacgtattgcacctgttaggtattcaaaataaacaattttacttaggtaatgactcggatcaacttctagagggtatataatgggggccagaacacatcgctaagcgaaagctaagctccagtacaggggcttttgtcaggttggcagaggaaaggtgaatccaatgggatccgcgtcaagtggagcaaggcaggtgg

SEQ ID NO: 678 - DNA, 10 bp, synthetic construct
  source 1..10: mol_type = other DNA
tcaggttggc

SEQ ID NO: 679 - DNA, 286 bp, synthetic construct
  source 1..286: mol_type = other DNA
gataagcttgatatcgcctgcagccgaattctcagcctgcatttctgccagggtatgtaaatataaaatcgttttcttatcttttggtgatcaggaagcagatgtgtagccatgattttccattgtagctcctcaaaggtcagcggatcaacttctagagggtatataatgggggccagaacacatcgctaagcgaaagctaagctccagtacaggggcttttgcagggccgtcagaggaaaggtgaatccaatgggatccgcgtcaagtggagcaaggcaggtgg

SEQ ID NO: 680 - DNA, 10 bp, synthetic construct
  source 1..10: mol_type = other DNA
cagggccgtc

SEQ ID NO: 681 - DNA, 286 bp, synthetic construct
  source 1..286: mol_type = other DNA
gataagcttgatatcgcctgcagccgaattctcagcctgcatttctgccagggttcttatctttatatccctcagctaatggaagatcggaaaatatttacataactccgctaaacaggtgcaagccaacaaactgacctttgcggatcaacttctagagggtatataatgggggccagaacacatcgctaagcgaaagctaagctccagtacaggggcttttgaaactgagccagaggaaaggtgaatccaatgggatccgcgtcaagtggagcaaggcaggtgg

SEQ ID NO: 682 - DNA, 10 bp, synthetic construct
  source 1..10: mol_type = other DNA
aaactgagcc

SEQ ID NO: 683 - DNA, 288 bp, synthetic construct
  source 1..288: mol_type = other DNA
gataagcttgatatcgcctgcagccgaattctcagcctgcatttctgccagggcaggaagcagatgtgtcttcgatatcaggttactcgaacgcacattttccaaagctccaatagtttcggtttcggtggtccaatccattagccggatcaacttctagagggtatataatgggggccagaacacatcgctaagcgaaagctaagctccagtacaggggcttttgccctatgaagagaggaaaggtgaatccaatgggatccgcgtcaagtggagcaaggcaggtgg

SEQ ID NO: 684 - DNA, 10 bp, synthetic construct
  source 1..10: mol_type = other DNA
ccctatgaag

SEQ ID NO: 685 - DNA, 288 bp, synthetic construct
  source 1..288: mol_type = other DNA
gataagcttgatatcgcctgcagccgaattctcagcctgcatttctgccagggtttcacaccttaactctctttatcaggttacagttacccgctaatgacttgccttgagaaaacaggtgcatggcccaccgaaaccgaaactacggatcaacttctagagggtatataatgggggccagaacacatcgctaagcgaaagctaagctccagtacaggggcttttgcctggcttccagaggaaaggtgaatccaatgggatccgcgtcaagtggagcaaggcaggtgg

SEQ ID NO: 686 - DNA, 10 bp, synthetic construct
  source 1..10: mol_type = other DNA
cctggcttcc

SEQ ID NO: 687 - protein, 6 aa, synthetic construct
  VARIANT 4..5: note = X can be any amino acid
DSGXXS

SEQ ID NO: 688 - protein, 4 aa, synthetic construct
KEGS

(19) United States
(12) Patent Application Publication
(10) Pub. No.: US 2025/0297276 A1
(43) Pub. Date: Sep. 25, 2025

(54) MACROPHAGE-SPECIFIC PROMOTERS AND USES THEREOF

(71) Applicant: Senti Biosciences, Inc., South San Francisco, CA (US)

(72) Inventors: Frances Deen Liu, San Mateo, CA (US); Michelle Elizabeth Hung, South San Francisco, CA (US); Assen Boyanov Roguev, San Francisco, CA (US); Myles Morgan Bliss Freeman, South San Francisco, CA (US)

(21) Appl. No.: 19/228,187

(22) Filed: Jun. 4, 2025

Related U.S. Application Data
(63) Continuation of application No. PCT/US23/82548, filed on Dec. 5, 2023.
(60) Provisional application No. 63/386,117, filed on Dec. 5, 2022.
(60) Provisional application No. 63/459,988, filed on Apr. 17, 2023.
(60) Provisional application No. 63/506,013, filed on Jun. 2, 2023.
(60) Provisional application No. 63/588,196, filed on Oct. 5, 2023.

(51) Int. Cl. C12N 15/85; A61K 48/00

(52) U.S. Cl. CPC C12N 15/85; A61K 48/0058; C12N 2830/008

(57) ABSTRACT

Described herein are compositions and methods for regulating expression of effector molecules using engineered macrophage-specific promoters. Immunoresponsive cells (such as macrophages) comprising the same are also described.

### CROSS REFERENCE TO RELATED APPLICATIONS

This application is a continuation of U.S. International Application No. PCT/US2023/082548, filed Dec. 5, 2023, which claims the benefit of U.S. Provisional Application Nos. 63/386,117, filed Dec. 5, 2022, 63/459,988, filed Apr. 17, 2023, 63/506,013, filed Jun. 2, 2023, and 63/588,196, filed Oct. 5, 2023, each of which is hereby incorporated by reference in their entirety for all purposes.

### SEQUENCE LISTING

The instant application contains a Sequence Listing which has been submitted via EFS-Web and is hereby incorporated by reference in its entirety. Said XML copy, created on Feb. 1, 2024, is named STB-046WO, and is 845,342 bytes in size.

### BACKGROUND

Cell-based therapy platforms provide promising avenues for treating a variety of diseases. Engineering of macrophages as cell therapies and drug delivery vehicles has become prominent as a potential immunotherapy. These engineered macrophages are typically genetically modified to express checkpoint inhibitors (e.g., PD-1/PD-L1 binders, SIRPα or CD47 blockers, etc.), immunomodulatory cytokines (e.g., interferons or interleukins), chimeric antigen receptors and/or other immune regulatory elements under control of a constitutive promoter. The constitutive expression of these engineered elements may not be desirable and may cause unwanted toxicities.

Given their promise, improvements in cell-based therapies are needed. An active area of exploration is engineering cell-based therapies to produce and/or secrete effector molecules such as cytokines, a process referred to as armoring, that enhance the cell-based therapy. Thus, additional methods of controlling and regulating the armoring of cell-based therapies, such as regulating production and/or secretion of payload effector molecules, are required.

### SUMMARY

This disclosure provides polarization-state specific promoters which enable the controlled expression of payloads only when macrophages encounter a given polarization cue. These polarization-state specific promoters not only provide selective payload expression but can also be used to prevent macrophage polarization plasticity.

Accordingly, in one aspect, described herein is an engineered macrophage-specific promoter system comprising: a regulatory element; and a heterologous payload; wherein the regulatory element exhibits greater activity in an M1 macrophage compared to an M2 or M0 macrophage, and wherein the regulatory element is or comprises an enhancer region that is derived from a promoter of a gene that is more highly expressed in M1 macrophage compared to M2 or M0 macrophages.

In some embodiments, the regulatory element is at least 100, 200, 300, 400, 500, 600, 700, 800, 900, 1000, 1100, 1200, 1300, 1400, 1500, 1600, 1700, 1800, 1900, 2000, 2200, 2400, 2500, 2600, 2800, or 3000 base pairs in length.

In some embodiments, the regulatory element is derived from a promoter of a gene, wherein the gene is selected from the group consisting of CCL19, CCR7, CXCL11, GBP5, IDO1, UBD, and UNQ6494.1. In some embodiments, the regulatory element is derived from a CCL19 promoter. In some embodiments, the regulatory element comprises a nucleotide sequence having at least 80%, 85%, 90%, 95%, 97.5%, 98%, 99%, or 100% identity to SEQ ID NO: 132. In some embodiments, the regulatory element is derived from a CCR7 promoter. In some embodiments, the regulatory element comprises a nucleotide sequence having at least 80%, 85%, 90%, 95%, 97.5%, 98%, 99%, or 100% identity to SEQ ID NO: 133. In some embodiments, the regulatory element is derived from a CXCL11 promoter. In some embodiments, the regulatory element comprises a nucleotide sequence having at least 80%, 85%, 90%, 95%, 97.5%, 98%, 99%, or 100% identity to SEQ ID NO: 134. In some embodiments, the regulatory element is derived from a GBP5 promoter. In some embodiments, the regulatory element comprises a nucleotide sequence having at least 80%, 85%, 90%, 95%, 97.5%, 98%, 99%, or 100% identity to SEQ ID NO: 135. In some embodiments, the regulatory element is derived from an IDO1 promoter. In some embodiments, the regulatory element comprises a nucleotide sequence having at least 80%, 85%, 90%, 95%, 97.5%, 98%, 99%, or 100% identity to SEQ ID NO: 136. In some embodiments, the regulatory element is derived from a UBD promoter. In some embodiments, the regulatory element comprises a nucleotide sequence having at least 80%, 85%, 90%, 95%, 97.5%, 98%, 99%, or 100% identity to SEQ ID NO: 137. In some embodiments, the regulatory element is derived from a UNQ6494.1 promoter. In some embodiments, the regulatory element comprises a nucleotide sequence having at least 80%, 85%, 90%, 95%, 97.5%, 98%, 99%, or 100% identity to SEQ ID NO: 138.

In some embodiments, the regulatory element: i. comprises a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256; and ii. does not comprise at least one repressive element selected from: SEQ ID NO: 226, SEQ ID NO: 234, SEQ ID NO: 236, SEQ ID NO: 238, SEQ ID NO: 246, and SEQ ID NO: 252. In some embodiments, the regulatory element further comprises a sixth transcriptional activating element as set forth in SEQ ID NO: 224 and/or a seventh transcriptional activating element as set forth in SEQ ID NO: 258. In some embodiments, the regulatory element further does not comprise SEQ ID NO: 228, SEQ ID NO: 230, SEQ ID NO: 232, SEQ ID NO: 242, SEQ ID NO:244, SEQ ID NO: 248, and SEQ ID NO: 250. In some embodiments, the regulatory element does not comprise the repressive elements as set forth in SEQ ID NO: 226, SEQ ID NO: 236, SEQ ID NO: 238, SEQ ID NO: 246, and SEQ ID NO: 252.

In some embodiments, the regulatory element comprises a sequence as set forth in GTTAAGTGGCTAGGGATAACATTGAGGCACTAAAGCATTATTGGTTCTGCAGTCA AGGGTAGGATAGATTGTTTTTTTTTTTTT (SEQ ID NO: 482), and a sequence as set forth in TTTGTGGTTTTATTGGTTTTCATATTACAAACAAAGAAACTAGAAAATGAAACCA TTCCAAAAGTGGAAGTAATTTCTCA (SEQ ID NO: 483). In some embodiments, the regulatory element comprises a sequence as set forth in GCTCTTCTAAAAATATGCGAAATGAGGTTTTTAGGGAGGTGTAGGTATGGCTGAA GAAAATCAAGGTGAATGAAGACAAGATCAATTGAGAATGTAGTTTCAGAAATAG CAAAGAAGCCAAAGTTTGAGGAAGTTAAGTGGCTAGGGATAACATTGAGGCACT AAAGCATTATTGGTTCTGCAGTCAAGGGTAGGATAGATTGTTTTTTTTTTTTTTGA GACGGAGTCTCACTCTGCTGCCCAGGC (SEQ ID NO: 484), a sequence as set forth in ATTTTGGTTTCAGTTTTCCTTAC (SEQ ID NO: 240), and a sequence as set forth in TTTGTGGTTTTATTGGTTTTCATATTACAAACAAAGAAACTAGAAAATGAAACCA TTCCAAAAGTGGAAGTAATTTCTCA (SEQ ID NO: 483).

In some embodiments, the regulatory element comprises a nucleotide sequence at least 80%, 85%, 90%, 95%, 97.5%, 98%, 99%, or 100% identical to SEQ ID NO: 456. In some embodiments, the regulatory element comprises a nucleotide sequence at least 80%, 85%, 90%, 95%, 97.5%, 98%, 99%, or 100% identical to SEQ ID NO: 457. In some embodiments, the regulatory element comprises a nucleotide sequence at least 80%, 85%, 90%, 95%, 97.5%, 98%, 99%, or 100% identical to SEQ ID NO: 458.

In some embodiments, the regulatory element: i. comprises a first transcriptional activating element as set forth in SEQ ID NO: 268 and a second transcriptional activating element as set forth in SEQ ID NO: 270; and ii. does not comprise at least one repressive element selected from: SEQ ID NO: 260, SEQ ID NO: 262, SEQ ID NO: 264, SEQ ID NO: 266, SEQ ID NO: 272, and SEQ ID NO: 391. In some embodiments, the regulatory element comprises at least one, at least two, at least three, at least four, or at least five tandem repeats of SEQ ID NO: 268 and SEQ ID NO: 270. In some embodiments, the regulatory element further comprises a third transcriptional activating element as set forth in SEQ ID NO: 291 and/or a fourth transcriptional activating element as set forth in: SEQ ID NO: 295. In some embodiments, the regulatory element does not comprise the repressive elements as set forth in SEQ ID NO: 262, SEQ ID NO: 264, SEQ ID NO: 272, and SEQ ID NO: 391, optionally wherein the regulatory element further does not comprise SEQ ID NO: 260 and/or SEQ ID NO: 266. In some embodiments, the regulatory element comprises a nucleotide sequence at least 80%, 85%, 90%, 95%, 97.5%, 98%, 99%, or 100% identical SEQ ID NO: 459. In some embodiments, the regulatory element comprises the nucleotide sequence as set forth in SEQ ID NO: 460. In some embodiments, the regulatory element comprises the nucleotide sequence as set forth in SEQ ID NO: 461.

In some embodiments, the regulatory element is operably linked to a minimal promoter, wherein optionally the minimal promoter comprises a sequence of a promoter selected from minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, SCP3, YB-SCP3, inducer molecule responsive promoters, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, hUBIb, and tandem repeats thereof. In some embodiments, the minimal promoter comprises a YB TATA promoter sequence.

In some embodiments, the regulatory element further comprises a translation initiator site, optionally wherein the translation initiator site is or comprises a Kozak sequence.

In some embodiments, the heterologous payload is selected from the group consisting of transcriptions factors, cytokines, receptors, enzymes, chemokines, antibodies, fragments of antibodies, miRNAs, and shRNAs.

In another aspect, provided herein is an engineered macrophage-specific promoter system comprising a regulatory element; and a heterologous payload; wherein the regulatory element exhibits greater activity in an M2 macrophage compared to an M1 or M0 macrophage, and wherein the regulatory element is or comprises an enhancer region that is derived from a promoter of a gene that is more highly expressed in M2 macrophage compared to M1 or M0 macrophages.

In some embodiments, the regulatory element is at least 100, 200, 300, 400, 500, 600, 700, 800, 900, 1000, 1100, 1200, 1300, 1400, 1500, 1600, 1700, 1800, 1900, 2000, 2200, 2400, 2500, 2600, 2800, or 3000 base pairs in length.

In some embodiments, the regulatory element is a promoter of a gene, wherein the gene is selected from the group consisting of CD28, SOCS3, PLXDC1, IL7R and ZNF704. In some embodiments, the regulatory element is derived from a CD28 promoter. In some embodiments, the regulatory element comprises a nucleotide sequence having at least 80%, 85%, 90%, 95%, 97.5%, 98%, 99%, or 100% identity to SEQ ID NO: 139. In some embodiments, the regulatory element is derived from a PLXDC1 promoter. In some embodiments, the regulatory element comprises a nucleotide sequence having at least 80%, 85%, 90%, 95%, 97.5%, 98%, 99%, or 100% identity to SEQ ID NO: 140. In some embodiments, the regulatory element is derived from a ZNF704 promoter. In some embodiments, the regulatory element comprises a nucleotide sequence having at least 80%, 85%, 90%, 95%, 97.5%, 98%, 99%, or 100% identity to SEQ ID NO: 141. In some embodiments, the regulatory element is derived from a IL7R promoter. In some embodiments, the regulatory element comprises a nucleotide sequence having at least 80%, 85%, 90%, 95%, 97.5%, 98%, 99%, or 100% identity to SEQ ID NO: 392. In some embodiments, the regulatory element is derived from a SOCS3 promoter. In some embodiments, the regulatory element comprises a nucleotide sequence having at least 80%, 85%, 90%, 95%, 97.5%, 98%, 99%, or 100% identity to SEQ ID NO: 393.

In some embodiments, the regulatory element is a promoter of a gene, wherein the gene is selected from the group consisting of: LNCAROD, MRC1, and ID3.

In some embodiments, the regulatory element is derived from a LNCAROD promoter. In some embodiments, the regulatory element derived from the LNCAROD promoter comprises: (i) a nucleotide sequence having at least 75%, at least 80%, at least 85%, at least 90%, at least 95%, at least 96%, at least 97%, at least 98%, at least 99% or 100% sequence identity to SEQ ID NO: 414; (ii) a nucleotide sequence having at least 75%, at least 80%, at least 85%, at least 90%, at least 95%, at least 96%, at least 97%, at least 98%, at least 99% or 100% sequence identity to SEQ ID NO: 415; (iii) a nucleotide sequence having at least 75%, at least 80%, at least 85%, at least 90%, at least 95%, at least 96%, at least 97%, at least 98%, at least 99% or 100% sequence identity to SEQ ID NO: 416; or (iv) a nucleotide sequence having at least 75%, at least 80%, at least 85%, at least 90%, at least 95%, at least 96%, at least 97%, at least 98%, at least 99% or 100% sequence identity to SEQ ID NO: 417.

In some embodiments, the regulatory element is derived from an ID3 promoter. In some embodiments, the regulatory element derived from the ID3 promoter comprises a nucleotide sequence having at least 75%, at least 80%, at least 85%, at least 90%, at least 95%, at least 96%, at least 97%, at least 98%, at least 99% or 100% sequence identity to SEQ ID NO: 418.

In some embodiments, the regulatory element is derived from an MRC1 promoter. In some embodiments, the regulatory element derived from the MRC1 promoter comprises a nucleotide sequence having at least 75%, at least 80%, at least 85%, at least 90%, at least 95%, at least 96%, at least 97%, at least 98%, at least 99% or 100% sequence identity to SEQ ID NO: 419.

In some embodiments, the heterologous payload is selected from the group consisting of transcriptions factors, cytokines, receptors, enzymes, chemokines, antibodies, fragments of antibodies, miRNAs, and shRNAs.

In another aspect, provided herein is an engineered macrophage-specific promoter comprising an ablation of at least one nucleotide motif, wherein the ablation increases specific activity of the engineered macrophage-specific promoter in M1 macrophages, as compared to activity of a corresponding engineered macrophage-specific promoter lacking the ablation in M1 macrophages.

In some embodiments, the wildtype macrophage promoter is a sequence selected from the group consisting of SEQ ID NOs: 132-138. In some embodiments, the wildtype macrophage promoter comprises the nucleotide sequence of SEQ ID NO: 132.

In some embodiments, the at least one nucleotide motif comprises a motif having a sequence within the nucleotide sequence of SEQ ID NO: 132, wherein the motif comprises a sequence selected from the group consisting of: position 63 to position 73 of SEQ ID NO: 132, position 80 to position 102 of SEQ ID NO: 132, position 141 to position 162 of SEQ ID NO: 132, position 212 to position 222 of SEQ ID NO: 132, position 229 to position 251 of SEQ ID NO: 132, position 307 to position 361 of SEQ ID NO: 132, position 365 to position 376 of SEQ ID NO: 132, position 559 to position 571 of SEQ ID NO: 132, position 617 to position 633 of SEQ ID NO: 132, position 782 to position 799 of SEQ ID NO: 132, position 852 to position 871 of SEQ ID NO: 132, position 886 to position 920 of SEQ ID NO: 132, position 933 to position 959 of SEQ ID NO: 132, position 1002 to position 1028 of SEQ ID NO: 132, position 1032 to position 1045 of SEQ ID NO: 132, position 1064 to position 1087 of SEQ ID NO: 132, position 1169 to position 1192 of SEQ ID NO: 132, position 1212 to position 1232 of SEQ ID NO: 132, position 1257 to position 1275 of SEQ ID NO: 132, position 1310 to position 1333 of SEQ ID NO: 132, position 1381 to position 1434 of SEQ ID NO: 132, position 1698 to position 1753 of SEQ ID NO: 132, position 1783 to position 1826 of SEQ ID NO: 132, position 1909 to position 1927 of SEQ ID NO: 132, position 1946 to position 1961 of SEQ ID NO: 132.

In some embodiments, the ablation comprises a substitution or deletion of one or more nucleotides of the at least one nucleotide motif.

In some embodiments, the at least one nucleotide motif comprises a motif having a sequence within the nucleotide sequence of SEQ ID NO: 132, wherein the motif corresponds to position 63 to position 73 of SEQ ID NO: 132.

In some embodiments, the ablation comprises a nucleotide substitution comprising the sequence CTTACCTACT (SEQ ID NO: 171) from position 63 to position 73 of SEQ ID NO: 132.

In some embodiments, the ablation comprises nucleotide deletions of position 63 to position 73 of SEQ ID NO: 132.

In some embodiments, the at least one nucleotide motif comprises a motif having a sequence within the nucleotide sequence of SEQ ID NO: 132, wherein the motif corresponds to position 80 to position 102 of SEQ ID NO: 132.

In some embodiments, the ablation comprises a nucleotide substitution comprising the sequence AATTCAGACGACAAACCATTCT (SEQ ID NO: 173) from position 80 to position 102 of SEQ ID NO: 132.

In some embodiments, the ablation comprises nucleotide deletions of position 80 to position 102 of SEQ ID NO: 132.

In some embodiments, the at least one nucleotide motif comprises a motif having a sequence within the nucleotide sequence of SEQ ID NO: 132, wherein the motif corresponds to position 141 to position 162 of SEQ ID NO: 132.

In some embodiments, the ablation comprises a nucleotide substitution comprising the sequence TTCTAAGTCCAATTCACGACA (SEQ ID NO:175) from position 141 to position 162 of SEQ ID NO:132.

In some embodiments, the ablation comprises nucleotide deletions of position 141 to position 162 of SEQ ID NO: 132.

In some embodiments, the at least one nucleotide motif comprises a motif having a sequence within the nucleotide sequence of SEQ ID NO: 132, wherein the motif corresponds to position 212 to position 222 of SEQ ID NO: 132.

In some embodiments, the ablation comprises a nucleotide substitution comprising the sequence GTTGAAGCTT (SEQ ID NO:177) from position 212 to position 222 of SEQ ID NO: 132.

In some embodiments, the ablation comprises nucleotide deletions of position 212 to position 222 of SEQ ID NO: 132.

In some embodiments, the at least one nucleotide motif comprises a motif having a sequence within the nucleotide sequence of SEQ ID NO: 132, wherein the motif corresponds to position 229 to position 251 of SEQ ID NO: 132.

In some embodiments, the ablation comprises a nucleotide substitution comprising the sequence GAGTCGTCAGACTCAATTATTA (SEQ ID NO:179) from position 229 to position 251 of SEQ ID NO: 132.

In some embodiments, the ablation comprises nucleotide deletions of position 229 to position 251 of SEQ ID NO: 132.

In some embodiments, the at least one nucleotide motif comprises a motif having a sequence within the nucleotide sequence of SEQ ID NO: 132, wherein the motif corresponds to position 307 to position 361 of SEQ ID NO: 132.

In some embodiments, the ablation comprises a nucleotide substitution comprising the sequence AATTGGAACCACGTATCTACTGCATTGTAACTACAACAGCTCGAGGTATTAGAT (SEQ ID NO:181) from position 307 to position 361 of SEQ ID NO: 132.

In some embodiments, the ablation comprises nucleotide deletions of position 307 to position 361 of SEQ ID NO: 132.

In some embodiments, the at least one nucleotide motif comprises a motif having a sequence within the nucleotide sequence of SEQ ID NO: 132, wherein the motif corresponds to position 365 to position 376 of SEQ ID NO: 132.

In some embodiments, the ablation comprises a nucleotide substitution comprising the sequence GGTGAATTTTC (SEQ ID NO:183) from position 365 to position 376 of SEQ ID NO: 132.

In some embodiments, the ablation comprises nucleotide deletions of position 365 to position 376 of SEQ ID NO: 132.

In some embodiments, the at least one nucleotide motif comprises a motif having a sequence within the nucleotide sequence of SEQ ID NO: 132, wherein the motif corresponds to position 559 to position 571 of SEQ ID NO: 132.

In some embodiments, the ablation comprises a nucleotide substitution comprising the sequence TACTCATCACTA (SEQ ID NO:185) from position 559 to position 571 of SEQ ID NO: 132.

In some embodiments, the ablation comprises nucleotide deletions of position 559 to position 571 of SEQ ID NO: 132.

In some embodiments, the at least one nucleotide motif comprises a motif having a sequence within the nucleotide sequence of SEQ ID NO: 132, wherein the motif corresponds to position 617 to position 633 of SEQ ID NO: 132.

In some embodiments, the ablation comprises a nucleotide substitution comprising the sequence TGCTAGTTGTCCAATA (SEQ ID NO:187) from position 617 to position 633 of SEQ ID NO: 132.

In some embodiments, the ablation comprises nucleotide deletions of position 617 to position 633 of SEQ ID NO: 132.

In some embodiments, the at least one nucleotide motif comprises a motif having a sequence within the nucleotide sequence of SEQ ID NO: 132, wherein the motif corresponds to position 782 to position 799 of SEQ ID NO: 132.

In some embodiments, the ablation comprises a nucleotide substitution comprising the sequence CGTGTGTCATATAGAAT (SEQ ID NO:189) from position 782 to position 799 of SEQ ID NO: 132.

In some embodiments, the ablation comprises nucleotide deletions of position 782 to position 799 of SEQ ID NO: 132.

In some embodiments, the at least one nucleotide motif comprises a motif having a sequence within the nucleotide sequence of SEQ ID NO: 132, wherein the motif corresponds to position 852 to position 871 of SEQ ID NO: 132.

In some embodiments, the ablation comprises a nucleotide substitution comprising the sequence AACAGTCTAAGTCCTCAAA (SEQ ID NO:191) from position 852 to position 871 of SEQ ID NO: 132.

In some embodiments, the ablation comprises nucleotide deletions of position 852 to position 871 of SEQ ID NO: 132.

In some embodiments, the at least one nucleotide motif comprises a motif having a sequence within the nucleotide sequence of SEQ ID NO: 132, wherein the motif corresponds to position 886 to position 920 of SEQ ID NO: 132.

In some embodiments, the ablation comprises a nucleotide substitution comprising the sequence ACTCTACGGAAGTAGCTTGTTTAAAACCTATAGT (SEQ ID NO:193) from position 886 to position 920 of SEQ ID NO: 132.

In some embodiments, the ablation comprises nucleotide deletions of position 886 to position 920 of SEQ ID NO: 132.

In some embodiments, the at least one nucleotide motif comprises a motif having a sequence within the nucleotide sequence of SEQ ID NO: 132, wherein the motif corresponds to position 933 to position 959 of SEQ ID NO: 132.

In some embodiments, the ablation comprises a nucleotide substitution comprising the sequence GTTCTACTAGTACAAAGGTACCAGTA (SEQ ID NO:195) from position 933 to position 959 of SEQ ID NO: 132.

In some embodiments, the ablation comprises nucleotide deletions of position 933 to position 959 of SEQ ID NO: 132.

In some embodiments, the at least one nucleotide motif comprises a motif having a sequence within the nucleotide sequence of SEQ ID NO: 132, wherein the motif corresponds to position 1002 to position 1028 of SEQ ID NO: 132.

In some embodiments, the ablation comprises a nucleotide substitution comprising the sequence TGAGTAAACTAACTTTCAACCGCTCT (SEQ ID NO:197) from position 1002 to position 1028 of SEQ ID NO: 132.

In some embodiments, the ablation comprises nucleotide deletions of position 1002 to position 1028 of SEQ ID NO: 132.

In some embodiments, the at least one nucleotide motif comprises a motif having a sequence within the nucleotide sequence of SEQ ID NO: 132, wherein the motif corresponds to position 1032 to position 1045 of SEQ ID NO: 132.

In some embodiments, the ablation comprises a nucleotide substitution comprising the sequence TCGTTACCATCTT (SEQ ID NO:199) from position 1032 to position 1045 of SEQ ID NO: 132.

In some embodiments, the ablation comprises nucleotide deletions of position 1032 to position 1045 of SEQ ID NO: 132.

In some embodiments, the at least one nucleotide motif comprises a motif having a sequence within the nucleotide sequence of SEQ ID NO: 132, wherein the motif corresponds to position 1064 to position 1087 of SEQ ID NO: 132.

In some embodiments, the ablation comprises a nucleotide substitution comprising the sequence AAACACCGTTTTGCTGTAATATC (SEQ ID NO:201) from position 1064 to position 1087 of SEQ ID NO: 132.

In some embodiments, the ablation comprises nucleotide deletions of position 1064 to position 1087 of SEQ ID NO: 132.

In some embodiments, the at least one nucleotide motif comprises a motif having a sequence within the nucleotide sequence of SEQ ID NO: 132, wherein the motif corresponds to position 1169 to position 1192 of SEQ ID NO: 132.

In some embodiments, the ablation comprises a nucleotide substitution comprising the sequence CGCGTAGAACTTCGTAACATTAA (SEQ ID NO:203) from position 1169 to position 1192 of SEQ ID NO: 132.

In some embodiments, the ablation comprises nucleotide deletions of position 1169 to position 1192 of SEQ ID NO: 132.

In some embodiments, the at least one nucleotide motif comprises a motif having a sequence within the nucleotide sequence of SEQ ID NO: 132, wherein the motif corresponds to position 1212 to position 1232 of SEQ ID NO: 132.

In some embodiments, the ablation comprises a nucleotide substitution comprising the sequence AGATAACGCCGTCATTGTAT (SEQ ID NO:205) from position 1212 to position 1232 of SEQ ID NO: 132.

In some embodiments, the ablation comprises nucleotide deletions of position 1212 to position 1232 of SEQ ID NO: 132.

In some embodiments, the at least one nucleotide motif comprises a motif having a sequence within the nucleotide sequence of SEQ ID NO: 132, wherein the motif corresponds to position 1257 to position 1275 of SEQ ID NO: 132.

In some embodiments, the ablation comprises a nucleotide substitution comprising the sequence TAACATCGTTCTCAGCTA (SEQ ID NO:207) from position 1257 to position 1275 of SEQ ID NO: 132.

In some embodiments, the ablation comprises nucleotide deletions of position 1257 to position 1275 of SEQ ID NO: 132.

In some embodiments, the at least one nucleotide motif comprises a motif having a sequence within the nucleotide sequence of SEQ ID NO: 132, wherein the motif corresponds to position 1310 to position 1333 of SEQ ID NO: 132.

In some embodiments, the ablation comprises a nucleotide substitution comprising the sequence ATATACAGTGTTCAGCGTGTTAC (SEQ ID NO:209) from position 1310 to position 1333 of SEQ ID NO: 132.

In some embodiments, the ablation comprises nucleotide deletions of position 1310 to position 1333 of SEQ ID NO: 132.

In some embodiments, the at least one nucleotide motif comprises a motif having a sequence within the nucleotide sequence of SEQ ID NO: 132, wherein the motif corresponds to position 1381 to position 1434 of SEQ ID NO: 132.

In some embodiments, the ablation comprises a nucleotide substitution comprising the sequence GACGTCTGTTAGTAGTATTACCCGTGTATTTCGGTCTTCGAGCAATTACTTTA (SEQ ID NO:211) from position 1381 to position 1434 of SEQ ID NO: 132.

In some embodiments, the ablation comprises nucleotide deletions of position 1381 to position 1434 of SEQ ID NO: 132.

In some embodiments, the at least one nucleotide motif comprises a motif having a sequence within the nucleotide sequence of SEQ ID NO: 132, wherein the motif corresponds to position 1698 to position 1753 of SEQ ID NO: 132.

In some embodiments, the ablation comprises a nucleotide substitution comprising the sequence GTGCATAAAAAGAAATTCACCACGAGTACCTATCTTGGTCTCGTTTGTTGCACTA (SEQ ID NO:213) from position 1698 to position 1753 of SEQ ID NO: 132.

In some embodiments, the ablation comprises nucleotide deletions of position 1698 to position 1753 of SEQ ID NO: 132.

In some embodiments, the at least one nucleotide motif comprises a motif having a sequence within the nucleotide sequence of SEQ ID NO: 132, wherein the motif corresponds to position 1783 to position 1826 of SEQ ID NO: 132.

In some embodiments, the ablation comprises a nucleotide substitution comprising the sequence AAAAACTACCAACCAGTTATCATTTCTCTGTGTAATATCTGAA (SEQ ID NO:215) from position 1783 to position 1826 of SEQ ID NO: 132.

In some embodiments, the ablation comprises nucleotide deletions of position 1783 to position 1826 of SEQ ID NO: 132.

In some embodiments, at least one nucleotide motif comprises a motif having a sequence within the nucleotide sequence of SEQ ID NO: 132, wherein the motif corresponds to position 1909 to position 1927 of SEQ ID NO: 132.

In some embodiments, the ablation comprises a nucleotide substitution comprising the sequence CGCAGAATATCGATATCT (SEQ ID NO:217) from position 1909 to position 1927 of SEQ ID NO: 132.

In some embodiments, the ablation comprises nucleotide deletions of position 1909 to position 1927 of SEQ ID NO: 132.

In some embodiments, the at least one nucleotide motif comprises a motif having a sequence within the nucleotide sequence of SEQ ID NO: 132, wherein the motif corresponds to position 1946 to position 1961 of SEQ ID NO: 132.

In some embodiments, the ablation comprises a nucleotide substitution comprising the sequence CGAATAGCACCTATA (SEQ ID NO:219) from position 1946 to position 1961 of SEQ ID NO: 132.

In some embodiments, the ablation comprises nucleotide deletions of position 1946 to position 1961 of SEQ ID NO: 132.

In some embodiments, the ablation comprises an ablation of at least two nucleotide motifs.

In some embodiments, the ablation comprises an ablation of at least three nucleotide motifs.

In some embodiments, the ablation comprises an ablation of at least four nucleotide motifs.

In some embodiments, the ablation comprises an ablation of at least five nucleotide motifs.

In some embodiments, the at least five nucleotide motifs comprise:
a nucleotide motif corresponding to position 365 to position 376 of SEQ ID NO: 132;
a nucleotide motif corresponding to position 1169 to position 1192 of SEQ ID NO: 132;
a nucleotide motif corresponding to position 1212 to position 1232 of SEQ ID NO: 132;
a nucleotide motif corresponding to position 1257 to position 1275 of SEQ ID NO: 132; and
a nucleotide motif corresponding to position 1381 to position 1434 of SEQ ID NO: 132.

In some embodiments, the ablation comprises a nucleotide substitution comprising the sequence GGTGAATTTTC (SEQ ID NO:183) from position 365 to position 376 of SEQ ID NO: 132.

In some embodiments, the ablation of the nucleotide motif corresponding to position 365 to position 376 of SEQ ID NO: 132 comprises a deletion of the nucleotide motif.

In some embodiments, the ablation comprises a nucleotide substitution comprising the sequence CGCGTAGAACTTCGTAACATTAA (SEQ ID NO:203) from position 1169 to position 1192 of SEQ ID NO: 132.

In some embodiments, the ablation of the nucleotide motif corresponding to position 1169 to position 1192 of SEQ ID NO:132 comprises a deletion of the nucleotide motif.

In some embodiments, the ablation comprises a nucleotide substitution comprising the sequence AGATAACGCCGTCATTGTAT (SEQ ID NO:205) from position 1212 to position 1232 of SEQ ID NO:132.

In some embodiments, the ablation of the nucleotide motif corresponding to position 1212 to position 1232 of SEQ ID NO:132 comprises a deletion of the nucleotide motif.

In some embodiments, the ablation comprises a nucleotide substitution comprising the sequence TAACATCGTTCTCAGCTA (SEQ ID NO:207) from position 1257 to position 1275 of SEQ ID NO:132.

In some embodiments, the ablation of the nucleotide motif corresponding to position 1257 to position 1275 of SEQ ID NO:132 comprises a deletion of the nucleotide motif.

In some embodiments, the ablation comprises a nucleotide substitution comprising the sequence GACGTCTGTTAGTAGTATTACCCGTGTATTTCGGTCTTCGAGCAATTACTTTA (SEQ ID NO:211) from position 1381 to position 1434 of SEQ ID NO:132.

In some embodiments, the ablation of the nucleotide motif corresponding to position 1381 to position 1434 of SEQ ID NO:132 comprises a deletion of the nucleotide motif.

In some embodiments, engineered macrophage-specific promoter comprises the nucleotide sequence of SEQ ID NO: 123.

In some embodiments, the engineered macrophage-specific promoter comprises the nucleotide sequence of SEQ ID NO: 125.

In some embodiments, the engineered macrophage-specific promoter further comprises an additional nucleotide motif selected from the group consisting of a sequence corresponding to position 1002 to position 1028 of SEQ ID NO: 132; a sequence corresponding to position 1310 to position 1333 of SEQ ID NO: 132; and a sequence corresponding to position 1909 to position 1927 of SEQ ID NO: 132.

In some embodiments, the ablation comprises an ablation of at least six nucleotide motifs.

In some embodiments, the ablation comprises an ablation of at least seven nucleotide motifs.

In some embodiments, the ablation comprises an ablation of at least eight nucleotide motifs.

In some embodiments, the at least eight nucleotide motifs comprise:
a nucleotide motif corresponding to position 365 to position 376 of SEQ ID NO: 132;
a nucleotide motif corresponding to position 1169 to position 1192 of SEQ ID NO: 132;
a nucleotide motif corresponding to position 1212 to position 1232 of SEQ ID NO: 132;
a nucleotide motif corresponding to position 1257 to position 1275 of SEQ ID NO: 132; and
a nucleotide motif corresponding to position 1381 to position 1434 of SEQ ID NO: 132.
a sequence corresponding to position 1002 to position 1028 of SEQ ID NO: 132;
a sequence corresponding to position 1310 to position 1333 of SEQ ID NO: 132; and
a sequence corresponding to position 1909 to position 1927 of SEQ ID NO: 132.

In some embodiments, the ablation comprises a nucleotide substitution comprising the sequence GGTGAATTTTC (SEQ ID NO:183) from position 365 to position 376 of SEQ ID NO:132.

In some embodiments, the ablation of the nucleotide motif corresponding to position 365 to position 376 of SEQ ID NO:132 comprises a deletion of the nucleotide motif.

In some embodiments, the ablation comprises a nucleotide substitution comprising the sequence CGCGTAGAACTTCGTAACATTAA (SEQ ID NO:203) from position 1169 to position 1192 of SEQ ID NO: 132.

In some embodiments, the ablation of the nucleotide motif corresponding to position 1169 to position 1192 of SEQ ID NO:132 comprises a deletion of the nucleotide motif.

In some embodiments, the ablation comprises a nucleotide substitution comprising the sequence AGATAACGCCGTCATTGTAT (SEQ ID NO:205) from position 1212 to position 1232 of SEQ ID NO: 132.

In some embodiments, the ablation of the nucleotide motif corresponding to position 1212 to position 1232 of SEQ ID NO:132 comprises a deletion of the nucleotide motif.

In some embodiments, the ablation comprises a nucleotide substitution comprising the sequence TAACATCGTTCTCAGCTA (SEQ ID NO:207) from position 1257 to position 1275 of SEQ ID NO:132.

In some embodiments, the ablation of the nucleotide motif corresponding to position 1257 to position 1275 of SEQ ID NO:132 comprises a deletion of the nucleotide motif.

In some embodiments, the ablation comprises a nucleotide substitution comprising the sequence GACGTCTGTTAGTAGTATTACCCGTGTATTTCGGTCTTCGAGCAATTACTTTA (SEQ ID NO:211) from position 1381 to position 1434 of SEQ ID NO:132.

In some embodiments, the ablation of the nucleotide motif corresponding to position 1381 to position 1434 of SEQ ID NO:132 comprises a deletion of the nucleotide motif.

In some embodiments, the ablation comprises a nucleotide substitution comprising the sequence TGAGTAAACTAACTTTCAACCGCTCT (SEQ ID NO:197) from position 1002 to position 1028 of SEQ ID NO:132.

In some embodiments, the ablation of the nucleotide motif corresponding to position 1002 to position 1028 of SEQ ID NO:132 comprises a deletion of the nucleotide motif.

In some embodiments, the ablation comprises a nucleotide substitution comprising the sequence ATATACAGTGTTCAGCGTGTTAC (SEQ ID NO:209) from position 1310 to position 1333 of SEQ ID NO:132.

In some embodiments, the ablation of the nucleotide motif corresponding to position 1310 to position 1333 of SEQ ID NO:132 comprises a deletion of the nucleotide motif.

In some embodiments, the ablation comprises a nucleotide substitution comprising the sequence CGCAGAATATCGATATCT (SEQ ID NO:217) from position 1909 to position 1927 of SEQ ID NO:132.

In some embodiments, the ablation of the nucleotide motif corresponding to position 1909 to position 1927 of SEQ ID NO:132 comprises a deletion of the nucleotide motif.

In some embodiments, the engineered macrophage-specific promoter comprises the nucleotide sequence of SEQ ID NO: 124.

In some embodiments, the engineered macrophage-specific promoter comprises the nucleotide sequence of SEQ ID NO: 126

In some embodiments, the wildtype macrophage promoter comprises the nucleotide sequence of SEQ ID NO: 136.

In some embodiments, the at least one nucleotide motif comprises a motif having a sequence within the nucleotide sequence of SEQ ID NO: 136, wherein the motif comprises a sequence selected from the group consisting of: to position 133 to position 144 of SEQ ID NO: 136, position 200 to 217 of SEQ ID NO: 136, position 225 to position 247 of SEQ ID NO: 136, position 303 to position 325 of SEQ ID NO: 136, position 332 to position 342 of SEQ ID NO: 136, position 391 to position 413 of SEQ ID NO: 136, position 423 to position 460 of SEQ ID NO: 136, position 467 to position 477 of SEQ ID NO: 136, position 693 to position 717 of SEQ ID NO: 136, position 738 to position 761 of SEQ ID NO: 136, position 838 to position 861 of SEQ ID NO: 136, position 1229 to position 1246 of SEQ ID NO: 136, position 1286 to position 1309 of SEQ ID NO: 136, position 1413 to position 1431 of SEQ ID NO: 136, position 1456 to position 1473 of SEQ ID NO: 136, to position 1530 to position 1544 of SEQ ID NO: 136, position 1577 to position 1590 of SEQ ID NO: 136, position 1816 to position 1836 of SEQ ID NO: 136, position 1852 to position 1872 of SEQ ID NO: 136, and to position 1876 to position to position 1896 of SEQ ID NO: 136.

In some embodiments, the ablation comprises a substitution or deletion of one or more nucleotides of the at least one nucleotide motif.

In some embodiments, the at least one nucleotide motif comprises a motif having a sequence within the nucleotide sequence of SEQ ID NO: 136, wherein the motif corresponds to position 133 to position 144 of SEQ ID NO: 136.

In some embodiments, the ablation comprises a nucleotide substitution comprising the sequence CTTACCTACTA (SEQ ID NO: 221) from position 133 to position 144 of SEQ ID NO: 136.

In some embodiments, the ablation comprises nucleotide deletions of position 133 to position 144 of SEQ ID NO: 136.

In some embodiments, the at least one nucleotide motif comprises a motif having a sequence within the nucleotide sequence of SEQ ID NO: 136, wherein the motif corresponds to position 200 to position 217 of SEQ ID NO: 136.

In some embodiments, the ablation comprises a nucleotide substitution comprising the sequence TAATTCGTCCGATAGAT (SEQ ID NO: 223) from position 200 to position 217 of SEQ ID NO: 136.

In some embodiments, the ablation comprises nucleotide deletions of position 200 to position 217 of SEQ ID NO: 136.

In some embodiments, the at least one nucleotide motif comprises a motif having a sequence within the nucleotide sequence of SEQ ID NO: 136, wherein the motif corresponds to position 225 to position 247 of SEQ ID NO: 136.

In some embodiments, the ablation comprises a nucleotide substitution comprising the sequence AATTCAGACGACAAACCATTCT (SEQ ID NO: 225) from position 225 to position 247 of SEQ ID NO: 136.

In some embodiments, the ablation comprises nucleotide deletions of position 225 to position 247 of SEQ ID NO: 136.

In some embodiments, the at least one nucleotide motif comprises a motif having a sequence within the nucleotide sequence of SEQ ID NO: 136, wherein the motif corresponds to position 303 to position 325 of SEQ ID NO: 136.

In some embodiments, the ablation comprises a nucleotide substitution comprising the sequence TTCTAAGTCCAATTCACGACAA (SEQ ID NO: 227) from position 303 to position 325 of SEQ ID NO: 136.

In some embodiments, the ablation comprises nucleotide deletions of position 303 to position 325 of SEQ ID NO: 136.

In some embodiments, the at least one nucleotide motif comprises a motif having a sequence within the nucleotide sequence of SEQ ID NO: 136, wherein the motif corresponds to position 332 to position 342 of SEQ ID NO: 136.

In some embodiments, the ablation comprises a nucleotide substitution comprising the sequence GTTGAAGCTT (SEQ ID NO: 229) from position 332 to position 342 of SEQ ID NO: 136.

In some embodiments, the ablation comprises nucleotide deletions of position 332 to position 342 of SEQ ID NO: 136.

In some embodiments, the at least one nucleotide motif comprises a motif having a sequence within the nucleotide sequence of SEQ ID NO: 136, wherein the motif corresponds to position 391 to position 413 of SEQ ID NO: 136.

In some embodiments, the ablation comprises a nucleotide substitution comprising the sequence AGTCGTCAGACTCAATTATTAC (SEQ ID NO: 231) from position 391 to position 413 of SEQ ID NO: 136.

In some embodiments, the ablation comprises nucleotide deletions of position 391 to position 413 of SEQ ID NO: 136.

In some embodiments, the at least one nucleotide motif comprises a motif having a sequence within the nucleotide sequence of SEQ ID NO: 136, wherein the motif corresponds to position 423 to position 460 of SEQ ID NO: 136.

In some embodiments, the ablation comprises a nucleotide substitution comprising the sequence TCCCTAGCGATCGAAGTTGATAAAACCTAAGTTTTGT (SEQ ID NO: 233) from position 423 to position 460 of SEQ ID NO: 136.

In some embodiments, the ablation comprises nucleotide deletions of position 423 to position 460 of SEQ ID NO: 136.

In some embodiments, the at least one nucleotide motif comprises a motif having a sequence within the nucleotide sequence of SEQ ID NO: 136, wherein the motif corresponds to position 467 to position 477 of SEQ ID NO: 136.

In some embodiments, the ablation comprises a nucleotide substitution comprising the sequence GCCTTCATAA (SEQ ID NO: 235) from position 467 to position 477 of SEQ ID NO: 136.

In some embodiments, the ablation comprises nucleotide deletions of position 467 to position 477 of SEQ ID NO: 136.

In some embodiments, the at least one nucleotide motif comprises a motif having a sequence within the nucleotide sequence of SEQ ID NO: 136, wherein the motif corresponds to position 693 to position 717 of SEQ ID NO: 136.

In some embodiments, the ablation comprises a nucleotide substitution comprising the sequence TCTCGCTAATAGGAGTAAGATACA (SEQ ID NO: 237) from position 693 to position 717 of SEQ ID NO: 136.

In some embodiments, the ablation comprises nucleotide deletions of position 693 to position 717 of SEQ ID NO: 136.

In some embodiments, the at least one nucleotide motif comprises a motif having a sequence within the nucleotide sequence of SEQ ID NO: 136, wherein the motif corresponds to position 738 to position 761 of SEQ ID NO: 136.

In some embodiments, the ablation comprises a nucleotide substitution comprising the sequence TTCTGCTGCAAGACCTATACTAT (SEQ ID NO: 239) from position 738 to position 761 of SEQ ID NO: 136.

In some embodiments, the ablation comprises nucleotide deletions of position 738 to position 761 of SEQ ID NO: 136.

In some embodiments, the at least one nucleotide motif comprises a motif having a sequence within the nucleotide sequence of SEQ ID NO: 136, wherein the motif corresponds to position 838 to position 861 of SEQ ID NO: 136.

In some embodiments, the ablation comprises a nucleotide substitution comprising the sequence CCACATTGCTATAGTGCTGTATA (SEQ ID NO: 241) from position 838 to position 861 of SEQ ID NO: 136.

In some embodiments, the ablation comprises nucleotide deletions of position 838 to position 861 of SEQ ID NO: 136.

In some embodiments, the at least one nucleotide motif comprises a motif having a sequence within the nucleotide sequence of SEQ ID NO: 136, wherein the motif corresponds to position 1229 to position 1246 of SEQ ID NO: 136.

In some embodiments, the ablation comprises a nucleotide substitution comprising the sequence TGCGTACCAGAATATTT (SEQ ID NO: 243) from position 1229 to position 1246 of SEQ ID NO: 136.

In some embodiments, the ablation comprises nucleotide deletions of position 1229 to position 1246 of SEQ ID NO: 136.

In some embodiments, the at least one nucleotide motif comprises a motif having a sequence within the nucleotide sequence of SEQ ID NO: 136, wherein the motif corresponds to position 1286 to position 1309 of SEQ ID NO: 136.

In some embodiments, the ablation comprises a nucleotide substitution comprising the sequence TGGTCACTATCACGTATATACCA (SEQ ID NO: 245) from position 1286 to position 1309 of SEQ ID NO: 136.

In some embodiments, the ablation comprises nucleotide deletions of position 1286 to position 1309 of SEQ ID NO: 136.

In some embodiments, the at least one nucleotide motif comprises a motif having a sequence within the nucleotide sequence of SEQ ID NO: 136, wherein the motif corresponds to position 1413 to position 1431 of SEQ ID NO: 136.

In some embodiments, the ablation comprises a nucleotide substitution comprising the sequence CGAGTTCGATAATACACT (SEQ ID NO: 247) from position 1413 to position 1431 of SEQ ID NO: 136.

In some embodiments, the ablation comprises nucleotide deletions of position 1413 to position 1431 of SEQ ID NO: 136.

In some embodiments, the at least one nucleotide motif comprises a motif having a sequence within the nucleotide sequence of SEQ ID NO: 136, wherein the motif corresponds to position 1456 to position 1473 of SEQ ID NO: 136.

In some embodiments, the ablation comprises a nucleotide substitution comprising the sequence AATACTGGTGCTTCAAT (SEQ ID NO: 249) from position 1456 to position 1473 of SEQ ID NO: 136.

In some embodiments, the ablation comprises nucleotide deletions of position 1456 to position 1473 of SEQ ID NO: 136.

In some embodiments, the at least one nucleotide motif comprises a motif having a sequence within the nucleotide sequence of SEQ ID NO: 136, wherein the motif corresponds to position 1530 to position 1544 of SEQ ID NO: 136.

In some embodiments, the ablation comprises a nucleotide substitution comprising the sequence CCGATAGAAAGAAT (SEQ ID NO: 251) from position 1530 to position 1544 of SEQ ID NO: 136.

In some embodiments, the ablation comprises nucleotide deletions of position 1530 to position 1544 of SEQ ID NO: 136.

In some embodiments, the at least one nucleotide motif comprises a motif having a sequence within the nucleotide sequence of SEQ ID NO: 136, wherein the motif corresponds to position 1577 to position 1590 of SEQ ID NO: 136.

In some embodiments, the ablation comprises a nucleotide substitution comprising the sequence TGTCTGTATAAAG (SEQ ID NO: 253) from position 1577 to position 1590 of SEQ ID NO: 136.

In some embodiments, the ablation comprises nucleotide deletions of position 1577 to position 1590 of SEQ ID NO: 136.

In some embodiments, the at least one nucleotide motif comprises a motif having a sequence within the nucleotide sequence of SEQ ID NO: 136, wherein the motif corresponds to position 1816 to position 1836 of SEQ ID NO: 136.

In some embodiments, the ablation comprises a nucleotide substitution comprising the sequence TGTTAAGCATACTAAACTGT (SEQ ID NO: 255) from position 1816 to position 1836 of SEQ ID NO: 136.

In some embodiments, the ablation comprises nucleotide deletions of position 1816 to position 1836 of SEQ ID NO: 136.

In some embodiments, the at least one nucleotide motif comprises a motif having a sequence within the nucleotide sequence of SEQ ID NO: 136, wherein the motif corresponds to position 1852 to position 1872 of SEQ ID NO: 136.

In some embodiments, the ablation comprises a nucleotide substitution comprising the sequence TTTCGAGCGACGCTTAATAT (SEQ ID NO: 257) from position 1852 to position 1872 of SEQ ID NO: 136.

In some embodiments, the ablation comprises nucleotide deletions of position 1852 to position 1872 of SEQ ID NO: 136.

In some embodiments, the at least one nucleotide motif comprises a motif having a sequence within the nucleotide sequence of SEQ ID NO: 136, wherein the motif corresponds to position 1876 to position to position 1896 of SEQ ID NO: 136.

In some embodiments, the ablation comprises a nucleotide substitution comprising the sequence TAGATAGTACGGGTTCCATA (SEQ ID NO: 259) from position 1876 to position to position 1896 of SEQ ID NO: 136.

In some embodiments, the ablation comprises nucleotide deletions of position 1876 to position to position 1896 of SEQ ID NO: 136.

In some embodiments, the wildtype macrophage promoter comprises the nucleotide sequence of SEQ ID NO: 137.

In some embodiments, the engineered macrophage-specific promoter: i. comprises a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256; and ii. does not comprise at least one repressive element selected from: SEQ ID NO: 226, SEQ ID NO: 234, SEQ ID NO: 236, SEQ ID NO: 238, SEQ ID NO: 246, and SEQ ID NO: 252. In some embodiments, the engineered macrophage-specific promoter further comprising a sixth transcriptional activating element as set forth in SEQ ID NO: 224 and/or a seventh transcriptional activating element as set forth in SEQ ID NO: 258. In some embodiments, the engineered macrophage-specific promoter further does not comprise SEQ ID NO: 228, SEQ ID NO: 230, SEQ ID NO: 232, SEQ ID NO: 242, SEQ ID NO:244, SEQ ID NO: 248, and SEQ ID NO: 250. In some embodiments, the engineered macrophage-specific promoter does not comprise the repressive elements as set forth in SEQ ID NO: 226, SEQ ID NO: 236, SEQ ID NO: 238, SEQ ID NO: 246, and SEQ ID NO: 252.

In some embodiments, the engineered macrophage-specific promoter comprises a sequence as set forth in GTTAAGTGGCTAGGGATAACATTGAGGCACTAAAGCATTATTGGTTCTGCAGTCA AGGGTAGGATAGATTGTTTTTTTTTTTTT (SEQ ID NO: 482), and a sequence as set forth in TTTGTGGTTTTATTGGTTTTCATATTACAAACAAAGAAACTAGAAAATGAAACCA TTCCAAAAGTGGAAGTAATTTCTCA (SEQ ID NO: 483). In some embodiments, the engineered macrophage-specific promoter comprises a sequence as set forth in GCTCTTCTAAAAATATGCGAAATGAGGTTTTTAGGGAGGTGTAGGTATGGCTGAA GAAAATCAAGGTGAATGAAGACAAGATCAATTGAGAATGTAGTTTCAGAAATAG CAAAGAAGCCAAAGTTTGAGGAAGTTAAGTGGCTAGGGATAACATTGAGGCACT AAAGCATTATTGGTTCTGCAGTCAAGGGTAGGATAGATTGTTTTTTTTTTTTTTGA GACGGAGTCTCACTCTGCTGCCCAGGC (SEQ ID NO: 484), a sequence as set forth in ATTTTGGTTTCAGTTTTCCTTAC (SEQ ID NO: 240), and a sequence as set forth in TTTGTGGTTTTATTGGTTTTCATATTACAAACAAAGAAACTAGAAAATGAAACCA TTCCAAAAGTGGAAGTAATTTCTCA (SEQ ID NO: 483).

In some embodiments, the engineered macrophage-specific promoter comprises a nucleotide sequence at least 80%, 85%, 90%, 95%, 97.5%, 98%, 99%, or 100% identical to SEQ ID NO: 456. In some embodiments, the engineered macrophage-specific promoter comprises a nucleotide sequence at least 80%, 85%, 90%, 95%, 97.5%, 98%, 99%, or 100% identical to SEQ ID NO: 457. In some embodiments, the engineered macrophage-specific promoter comprises a nucleotide sequence at least 80%, 85%, 90%, 95%, 97.5%, 98%, 99%, or 100% identical to SEQ ID NO: 458.

In some embodiments, the at least one nucleotide motif comprises a motif having a sequence within the nucleotide sequence of SEQ ID NO: 137, wherein the motif comprises a sequence selected from the group consisting of: to position 43 to position 60, position 107 to position 120 of SEQ ID NO: 137, position 210 to position 230 of SEQ ID NO: 137, position 345 to position 407 of SEQ ID NO: 137, position 427 to position 457 of SEQ ID NO: 137, position 468 to position 484 of SEQ ID NO: 137, position 560 to position 582, position 730 to position 746 of SEQ ID NO: 137, position 809 to position 820 of SEQ ID NO: 137, position 827 to position 837 of SEQ ID NO: 137, position 858 to position 878 of SEQ ID NO: 137, position 1291 to position 1302 of SEQ ID NO: 137, position 1321 to position 1341 of SEQ ID NO: 137, position 1435 to position 1463 of SEQ ID NO: 137, position 1530 to position 1541 of SEQ ID NO: 137, position 1707 to position 1718 of SEQ ID NO: 137, position 1834 to position 1863 of SEQ ID NO: 137, position 1870 to position 1882 of SEQ ID NO: 137, and to position 1913 to position 1929 of SEQ ID NO: 137.

In some embodiments, the engineered macrophage-specific promoter: i. comprises a first transcriptional activating element as set forth in SEQ ID NO: 268 and a second transcriptional activating element as set forth in SEQ ID NO: 270; and ii. does not comprise at least one repressive element selected from: SEQ ID NO: 260, SEQ ID NO: 262, SEQ ID NO: 264, SEQ ID NO: 266, SEQ ID NO: 272, and SEQ ID NO: 391. In some embodiments, the engineered macrophage-specific promoter comprises at least one, at least two, at least three, at least four, or at least five tandem repeats of SEQ ID NO: 268 and SEQ ID NO: 270. In some embodiments, the engineered macrophage-specific promoter further comprises a third transcriptional activating element as set forth in SEQ ID NO: 291 and/or a fourth transcriptional activating element as set forth in: SEQ ID NO: 295. In some embodiments, the engineered macrophage-specific promoter does not comprise the repressive elements as set forth in SEQ ID NO: 262, SEQ ID NO: 264, SEQ ID NO: 272, and SEQ ID NO: 391, optionally wherein the engineered macrophage-specific promoter further does not comprise SEQ ID NO: 260 and/or SEQ ID NO: 266. In some embodiments, the engineered macrophage-specific promoter comprises a nucleotide sequence at least 80%, 85%, 90%, 95%, 97.5%, 98%, 99%, or 100% identical SEQ ID NO: 459. In some embodiments, the engineered macrophage-specific promoter comprises the nucleotide sequence as set forth in SEQ ID NO: 460. In some embodiments, the engineered macrophage-specific promoter comprises the nucleotide sequence as set forth in SEQ ID NO: 461.

In some embodiments, the engineered macrophage-specific promoter is operably linked to a minimal promoter, wherein optionally the minimal promoter comprises a sequence of a promoter selected from minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, SCP3, YB-SCP3, inducer molecule responsive promoters, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, hUBIb, and tandem repeats thereof. In some embodiments, the minimal promoter comprises a YB TATA promoter sequence.

In some embodiments, the engineered macrophage-specific promoter further comprises a translation initiator site, optionally wherein the translation initiator site is or comprises a Kozak sequence.

In some embodiments, the ablation comprises a substitution or deletion of one or more nucleotides of the at least one nucleotide motif.

In some embodiments, the at least one nucleotide motif comprises a motif having a sequence within the nucleotide sequence of SEQ ID NO: 137, wherein the motif corresponds to position 43 to position 60 of SEQ ID NO: 137.

In some embodiments, the ablation comprises a nucleotide substitution comprising the sequence CTTACCTACTAGGTTAA (SEQ ID NO: 261) from position 43 to position 60 of SEQ ID NO: 137.

In some embodiments, the ablation comprises nucleotide deletions of position 43 to position 60 of SEQ ID NO: 137.

In some embodiments, the at least one nucleotide motif comprises a motif having a sequence within the nucleotide sequence of SEQ ID NO: 137, wherein the motif corresponds to position 107 to position 120 of SEQ ID NO: 137.

In some embodiments, the ablation comprises a nucleotide substitution comprising the sequence ACTCGAATTCAGA (SEQ ID NO: 263) from position 107 to position 120 of SEQ ID NO: 137.

In some embodiments, the ablation comprises nucleotide deletions of position 107 to position 120 of SEQ ID NO: 137.

In some embodiments, the at least one nucleotide motif comprises a motif having a sequence within the nucleotide sequence of SEQ ID NO: 137, wherein the motif corresponds to position 210 to position 230 of SEQ ID NO: 137.

In some embodiments, the ablation comprises a nucleotide substitution comprising the sequence ATTCTAGCCTTACAGCCTAA (SEQ ID NO: 265) from position 210 to position 230 of SEQ ID NO: 137.

In some embodiments, the ablation comprises nucleotide deletions of position 210 to position 230 of SEQ ID NO: 137.

In some embodiments, the at least one nucleotide motif comprises a motif having a sequence within the nucleotide sequence of SEQ ID NO: 137, wherein the motif corresponds to position 345 to position 407 of SEQ ID NO: 137.

In some embodiments, the ablation comprises a nucleotide substitution comprising the sequence ACTCTACGGAAGTAGCTTGTTTAAAACCTATAGTCTCTTCGGAGTCGTTCTACTA GTACAAA (SEQ ID NO: 267) from position 345 to position 407 of SEQ ID NO: 137.

In some embodiments, the ablation comprises nucleotide deletions of position 345 to position 407 of SEQ ID NO: 137.

In some embodiments, the at least one nucleotide motif comprises a motif having a sequence within the nucleotide sequence of SEQ ID NO: 137, wherein the motif corresponds to position 427 to position 457 of SEQ ID NO: 137.

In some embodiments, the ablation comprises a nucleotide substitution comprising the sequence TGAGTAAACTAACTTTCAACCGCTCTTCGT (SEQ ID NO: 269) from position 427 to position 457 of SEQ ID NO: 137.

In some embodiments, the ablation comprises nucleotide deletions of 427 to position 457 of SEQ ID NO: 137.

In some embodiments, the at least one nucleotide motif comprises a motif having a sequence within the nucleotide sequence of SEQ ID NO: 137, wherein the motif corresponds to position 468 to position 484 of SEQ ID NO: 137.

In some embodiments, the ablation comprises a nucleotide substitution comprising the sequence CTTAAACACCGTTTTG (SEQ ID NO: 271) from position 468 to position 484 of SEQ ID NO: 137.

In some embodiments, the ablation comprises nucleotide deletions of position 468 to position 484 of SEQ ID NO: 137.

In some embodiments, the at least one nucleotide motif comprises a motif having a sequence within the nucleotide sequence of SEQ ID NO: 137, wherein the motif corresponds to position 560 to position 582 of SEQ ID NO: 137.

In some embodiments, the ablation comprises a nucleotide substitution comprising the sequence CTGTAATATCATCCGCTCTTTA (SEQ ID NO: 273) from position 560 to position 582 of SEQ ID NO: 137.

In some embodiments, the ablation comprises nucleotide deletions of position 560 to position 582 of SEQ ID NO: 137.

In some embodiments, the at least one nucleotide motif comprises a motif having a sequence within the nucleotide sequence of SEQ ID NO: 137, wherein the motif corresponds to position 730 to position 746 of SEQ ID NO: 137.

In some embodiments, the ablation comprises a nucleotide substitution comprising the sequence TGATCGGCCAATATTT (SEQ ID NO: 274) from position 730 to position 746 of SEQ ID NO: 137.

In some embodiments, the ablation comprises nucleotide deletions of position 730 to position 746 of SEQ ID NO: 137.

In some embodiments, the at least one nucleotide motif comprises a motif having a sequence within the nucleotide sequence of SEQ ID NO: 137, wherein the motif corresponds to position 809 to position 820 of SEQ ID NO: 137.

In some embodiments, the ablation comprises a nucleotide substitution comprising the sequence TAGAACTTCGT (SEQ ID NO: 276) from position 809 to position 820 of SEQ ID NO: 137.

In some embodiments, the ablation comprises nucleotide deletions of position 809 to position 820 of SEQ ID NO: 137.

In some embodiments, the at least one nucleotide motif comprises a motif having a sequence within the nucleotide sequence of SEQ ID NO: 137, wherein the motif corresponds to position 827 to position 837 of SEQ ID NO: 137.

In some embodiments, the ablation comprises a nucleotide substitution comprising the sequence AACATTAAGT (SEQ ID NO: 278) from position 827 to position 837 of SEQ ID NO: 137.

In some embodiments, the ablation comprises nucleotide deletions of position 827 to position 837 of SEQ ID NO: 137.

In some embodiments, the at least one nucleotide motif comprises a motif having a sequence within the nucleotide sequence of SEQ ID NO: 137, wherein the motif corresponds to position 858 to position 878 of SEQ ID NO: 137.

In some embodiments, the ablation comprises a nucleotide substitution comprising the sequence TAGATAACGCCGTCATTGTA (SEQ ID NO: 280) from position 858 to position 878 of SEQ ID NO: 137.

In some embodiments, the ablation comprises nucleotide deletions of position 858 to position 878 of SEQ ID NO: 137.

In some embodiments, the at least one nucleotide motif comprises a motif having a sequence within the nucleotide sequence of SEQ ID NO: 137, wherein the motif corresponds to position 1291 to position 1302 of SEQ ID NO: 137.

In some embodiments, the ablation comprises a nucleotide substitution comprising the sequence TTTCTCTAACG (SEQ ID NO: 282) from position 1291 to position 1302 of SEQ ID NO: 137.

In some embodiments, the ablation comprises nucleotide deletions of position 1291 to position 1302 of SEQ ID NO: 137.

In some embodiments, the at least one nucleotide motif comprises a motif having a sequence within the nucleotide sequence of SEQ ID NO: 137, wherein the motif corresponds to position 1321 to position 1341 of SEQ ID NO: 137.

In some embodiments, the ablation comprises a nucleotide substitution comprising the sequence CTAACATCGTTCTCAGCTAA (SEQ ID NO: 284) from position 1321 to position 1341 of SEQ ID NO: 137.

In some embodiments, the ablation comprises nucleotide deletions of position 1321 to position 1341 of SEQ ID NO: 137.

In some embodiments, the at least one nucleotide motif comprises a motif having a sequence within the nucleotide sequence of SEQ ID NO: 137, wherein the motif corresponds to position 1435 to position 1463 of SEQ ID NO: 137.

In some embodiments, the ablation comprises a nucleotide substitution comprising the sequence TATACAGTGTTCAGCGTGTTACTTGTGA (SEQ ID NO: 286) from position 1435 to position 1463 of SEQ ID NO: 137.

In some embodiments, the ablation comprises nucleotide deletions of position 1435 to position 1463 of SEQ ID NO: 137.

In some embodiments, the at least one nucleotide motif comprises a motif having a sequence within the nucleotide sequence of SEQ ID NO: 137, wherein the motif corresponds to position 1530 to position 1541 of SEQ ID NO: 137.

In some embodiments, the ablation comprises a nucleotide substitution comprising the sequence CGTACAAGTAT (SEQ ID NO: 288) from position 1530 to position 1541 of SEQ ID NO: 137.

In some embodiments, the ablation comprises nucleotide deletions of position 1530 to position 1541 of SEQ ID NO: 137.

In some embodiments, the at least one nucleotide motif comprises a motif having a sequence within the nucleotide sequence of SEQ ID NO: 137, wherein the motif corresponds to position 1707 to position 1718 of SEQ ID NO: 137.

In some embodiments, the ablation comprises a nucleotide substitution comprising the sequence AGTCTCTGAAT (SEQ ID NO: 290) from position 1707 to position 1718 of SEQ ID NO: 137.

In some embodiments, the ablation comprises nucleotide deletions of position 1707 to position 1718 of SEQ ID NO: 137.

In some embodiments, the at least one nucleotide motif comprises a motif having a sequence within the nucleotide sequence of SEQ ID NO: 137, wherein the motif corresponds to position 1834 to position 1863 of SEQ ID NO: 137.

In some embodiments, the ablation comprises a nucleotide substitution comprising the sequence CCCTATATAATACCCGCTAGCATACAAAT (SEQ ID NO: 292) from position 1834 to position 1863 of SEQ ID NO: 137.

In some embodiments, the ablation comprises nucleotide deletions of position 1834 to position 1863 of SEQ ID NO: 137.

In some embodiments, the at least one nucleotide motif comprises a motif having a sequence within the nucleotide sequence of SEQ ID NO: 137, wherein the motif corresponds to position 1870 to position 1882 of SEQ ID NO: 137.

In some embodiments, the ablation comprises a nucleotide substitution comprising the sequence GTTGCTCATATA (SEQ ID NO: 294) from position 1870 to position 1882of SEQ ID NO: 137.

In some embodiments, the ablation comprises nucleotide deletions of position 1870 to position 1882 of SEQ ID NO: 137.

In some embodiments, the at least one nucleotide motif comprises a motif having a sequence within the nucleotide sequence of SEQ ID NO: 137, wherein the motif corresponds to position 1913 to position 1929 of SEQ ID NO: 137.

In some embodiments, the ablation comprises a nucleotide substitution comprising the sequence ACGTCTGTTAGTAGTA (SEQ ID NO: 296) from position 1913 to position 1929 of SEQ ID NO: 137.

In some embodiments, the ablation comprises nucleotide deletions of position 1913 to position 1929 of SEQ ID NO: 137.

In another aspect, provided herein is an engineered macrophage-specific promoter comprising an ablation of at least one nucleotide motif, wherein the ablation increases specific activity of the engineered macrophage-specific promoter in M2 macrophages, as compared to activity of a corresponding engineered macrophage-specific promoter lacking the ablation in M2 macrophages.

In some embodiments, the wildtype macrophage promoter is a sequence selected from the group consisting of SEQ ID NOs 139-141, 392, and 393.

In another aspect, provided herein is an engineered macrophage-specific promoter comprising at least one regulatory element, wherein the regulatory element exhibits greater activity in an M1 macrophage compared to an M2 or M0 macrophage or exhibits greater activity in an M2 macrophage compared to an M1 or M0 macrophage.

In some embodiments, the engineered macrophage-specific promoter comprises at least 2, at least 3, at least 4, or at least 5 regulatory elements. In some embodiments, the engineered macrophage-specific promoter comprises at least 5 regulatory elements. In some embodiments, each of the at least 5 regulatory elements are different. In some embodiments, each of the at least 5 regulatory elements are the same.

In some embodiments, the engineered macrophage-specific promoter exhibits increased activity in M1 macrophage compared to M2 macrophages.

In some embodiments, the engineered macrophage-specific promoter comprises a nucleotide sequence selected from the group consisting of SEQ ID NOs: 297-313 and SEQ ID NOs: 372-390.

In some embodiments, the engineered macrophage-specific promoter comprises a nucleotide sequence selected from: (i) a nucleotide sequence having at least 75%, at least 80%, at least 85%, at least 90%, at least 95%, at least 96%, at least 97%, at least 98%, at least 99% or 100% sequence identity to SEQ ID NO: 440; (ii) a nucleotide sequence having at least 75%, at least 80%, at least 85%, at least 90%, at least 95%, at least 96%, at least 97%, at least 98%, at least 99% or 100% sequence identity to SEQ ID NO: 441; (iii) a nucleotide sequence having at least 75%, at least 80%, at least 85%, at least 90%, at least 95%, at least 96%, at least 97%, at least 98%, at least 99% or 100% sequence identity to SEQ ID NO: 442; and (iv) a nucleotide sequence having at least 75%, at least 80%, at least 85%, at least 90%, at least 95%, at least 96%, at least 97%, at least 98%, at least 99% or 100% sequence identity to SEQ ID NO: 443.

In some embodiments, the engineered macrophage-specific promoter exhibits increased activity in M2 macrophages compared to M1 macrophages, M0 macrophages, or both M1 and M0 macrophages.

In some embodiments, the engineered macrophage-specific promoter comprises a nucleotide sequence having at least 80%, 85%, 90%, 95%, 97.5%, 98%, 99%, or 100% identity to a nucleotide sequence selected from the group consisting of SEQ ID NOs: 314-371.

In some embodiments, the engineered macrophage-specific promoter comprises a nucleotide sequence selected from: (i) a nucleotide sequence having at least 75%, at least 80%, at least 85%, at least 90%, at least 95%, at least 96%, at least 97%, at least 98%, at least 99% or 100% sequence identity to SEQ ID NO: 420; (ii) a nucleotide sequence having at least 75%, at least 80%, at least 85%, at least 90%, at least 95%, at least 96%, at least 97%, at least 98%, at least 99% or 100% sequence identity to SEQ ID NO: 421; (iii) a nucleotide sequence having at least 75%, at least 80%, at least 85%, at least 90%, at least 95%, at least 96%, at least 97%, at least 98%, at least 99% or 100% sequence identity to SEQ ID NO: 422; (iv) a nucleotide sequence having at least 75%, at least 80%, at least 85%, at least 90%, at least 95%, at least 96%, at least 97%, at least 98%, at least 99% or 100% sequence identity to SEQ ID NO: 423; (v) a nucleotide sequence having at least 75%, at least 80%, at least 85%, at least 90%, at least 95%, at least 96%, at least 97%, at least 98%, at least 99% or 100% sequence identity to SEQ ID NO: 424; (vi) a nucleotide sequence having at least 75%, at least 80%, at least 85%, at least 90%, at least 95%, at least 96%, at least 97%, at least 98%, at least 99% or 100% sequence identity to SEQ ID NO: 425; (vii) a nucleotide sequence having at least 75%, at least 80%, at least 85%, at least 90%, at least 95%, at least 96%, at least 97%, at least 98%, at least 99% or 100% sequence identity to SEQ ID NO: 426; (viii) a nucleotide sequence having at least 75%, at least 80%, at least 85%, at least 90%, at least 95%, at least 96%, at least 97%, at least 98%, at least 99% or 100% sequence identity to SEQ ID NO: 427; (ix) a nucleotide sequence having at least 75%, at least 80%, at least 85%, at least 90%, at least 95%, at least 96%, at least 97%, at least 98%, at least 99% or 100% sequence identity to SEQ ID NO: 428; (x) a nucleotide sequence having at least 75%, at least 80%, at least 85%, at least 90%, at least 95%, at least 96%, at least 97%, at least 98%, at least 99% or 100% sequence identity to SEQ ID NO: 429; (xi) a nucleotide sequence having at least 75%, at least 80%, at least 85%, at least 90%, at least 95%, at least 96%, at least 97%, at least 98%, at least 99% or 100% sequence identity to SEQ ID NO: 430; (xii) a nucleotide sequence having at least 75%, at least 80%, at least 85%, at least 90%, at least 95%, at least 96%, at least 97%, at least 98%, at least 99% or 100% sequence identity to SEQ ID NO: 431; (xiii) a nucleotide sequence having at least 75%, at least 80%, at least 85%, at least 90%, at least 95%, at least 96%, at least 97%, at least 98%, at least 99% or 100% sequence identity to SEQ ID NO: 432; (xiv) a nucleotide sequence having at least 75%, at least 80%, at least 85%, at least 90%, at least 95%, at least 96%, at least 97%, at least 98%, at least 99% or 100% sequence identity to SEQ ID NO: 433; (xv) a nucleotide sequence having at least 75%, at least 80%, at least 85%, at least 90%, at least 95%, at least 96%, at least 97%, at least 98%, at least 99% or 100% sequence identity to SEQ ID NO: 434; (xvi) a nucleotide sequence having at least 75%, at least 80%, at least 85%, at least 90%, at least 95%, at least 96%, at least 97%, at least 98%, at least 99% or 100% sequence identity to SEQ ID NO: 435; (xvii) a nucleotide sequence having at least 75%, at least 80%, at least 85%, at least 90%, at least 95%, at least 96%, at least 97%, at least 98%, at least 99% or 100% sequence identity to SEQ ID NO: 436; (xviii) a nucleotide sequence having at least 75%, at least 80%, at least 85%, at least 90%, at least 95%, at least 96%, at least 97%, at least 98%, at least 99% or 100% sequence identity to SEQ ID NO: 437; (xix) a nucleotide sequence having at least 75%, at least 80%, at least 85%, at least 90%, at least 95%, at least 96%, at least 97%, at least 98%, at least 99% or 100% sequence identity to SEQ ID NO: 438, and (xx) a nucleotide sequence having at least 75%, at least 80%, at least 85%, at least 90%, at least 95%, at least 96%, at least 97%, at least 98%, at least 99% or 100% sequence identity to SEQ ID NO: 439.

In some embodiments, the engineered macrophage-specific promoter further comprises a minimal promoter operably linked to the engineered macrophage-specific promoter. In some embodiments, the minimal promoter is derived from a promoter selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, inducer molecule responsive promoters, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, hUBIb, and tandem repeats thereof.

In some embodiments, the engineered macrophage-specific promoter comprise at least one regulatory element, wherein: i. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 420, and the minimal promoter comprises a sequence of a promoter selected from: minTK promoter; an SCP3 promoter, and a hybrid YBTATA-SCP3 (“YB-SCP3”) promoter; ii. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 425, and the minimal promoter comprises a sequence of a promoter selected from: a minTK promoter, an SCP3 promoter, a YB-SCP3 promoter, a YBTATA promoter, and a minCMV promoter; iii. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 426, and the minimal promoter comprises a sequence of a YB-SCP3 promoter; iv. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 427, and the minimal promoter comprises a sequence of a minCMV promoter; or v. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 423, and the minimal promoter comprises a sequence of a minTK promoter.

In some embodiments, the minTK comprises a nucleotide sequence having at least 80% identity to SEQ ID NO: 448. In some embodiments, the minTK comprises a nucleotide sequence as set forth in SEQ ID NO: 448. In some embodiments, the SCP3 comprises a nucleotide sequence having at least 80% identity to SEQ ID NO: 449. In some embodiments, the SCP3 comprises a nucleotide sequence as set forth in SEQ ID NO: 449. In some embodiments, the YB-SCP3 comprises a nucleotide sequence having at least 80% identity to SEQ ID NO: 450. In some embodiments, the YB-SCP3 comprises a nucleotide sequence as set forth in SEQ ID NO: 450. In some embodiments, the minCMV comprises a nucleotide sequence having at least 80% identity to SEQ ID NO: 447. In some embodiments, the minCMV comprises a nucleotide sequence as set forth in SEQ ID NO: 447.

In some embodiments, the engineered macrophage-specific promoter comprises at least one regulatory element, wherein the at least one regulatory element comprises a nucleotide sequence having at least 95% sequence identity to SEQ ID NO: 420. In some embodiments, the at least one regulatory element comprises a nucleotide sequence as set forth in SEQ ID NO: 420.

In some embodiments, the engineered macrophage-specific promoter comprises at least one regulatory element, wherein the at least one regulatory element comprises a nucleotide sequence having at least 95% sequence identity to SEQ ID NO: 425. In some embodiments, the at least one regulatory element comprises a nucleotide sequence as set forth in SEQ ID NO: 425.

In some embodiments, the engineered macrophage-specific promoter comprises at least one regulatory element, wherein the at least one regulatory element comprises a nucleotide sequence having at least 95% sequence identity to SEQ ID NO: 426. In some embodiments, the at least one regulatory element comprises a nucleotide sequence as set forth in SEQ ID NO: 426.

In some embodiments, the engineered macrophage-specific promoter comprises at least one regulatory element, wherein the at least one regulatory element comprises a nucleotide sequence having at least 95% sequence identity to SEQ ID NO: 427. In some embodiments, the at least one regulatory element comprises a nucleotide sequence as set forth in SEQ ID NO: 427.

In some embodiments, the engineered macrophage-specific promoter comprises at least one regulatory element, wherein the at least one regulatory element comprises a nucleotide sequence having at least 95% sequence identity to SEQ ID NO: 423. In some embodiments, the at least one regulatory element comprises a nucleotide sequence as set forth in SEQ ID NO: 423.

In some embodiments, the minimal promoter further comprises a flanking sequence.

In some embodiments, the engineered macrophage-specific promoter further comprises at least one inert sequence. In some embodiments, the inert sequence is derived from an insulating element.

In some embodiments, the engineered macrophage-specific promoter further comprises at least one molecular barcode.

In some embodiments, M2 macrophages are selected from the group consisting of M2a macrophages, M2b macrophages, and M2c macrophages.

In another aspect, provided herein is an engineered macrophage-specific promoter system comprising at least one regulatory element and a heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 132.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 133.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 134.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 135.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 136.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 137.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 138.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 139.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 140.

In another aspect, provided herein is an engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 141.

In another aspect, provided herein is an engineered macrophage-specific promoter system comprising at least one regulatory element and at least one heterologous payload, comprising a nucleotide sequence having at least 75%, at least 80%, at least 85%, at least 90%, at least 95%, at least 96%, at least 97%, at least 98%, at least 99% or 100% sequence identity to SEQ ID NO: 392.

In another aspect, provided herein is an engineered macrophage-specific promoter system comprising at least one regulatory element and at least one heterologous payload, comprising a nucleotide sequence having at least 75%, at least 80%, at least 85%, at least 90%, at least 95%, at least 96%, at least 97%, at least 98%, at least 99% or 100% sequence identity to SEQ ID NO: 393.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 142.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 143.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 144.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 145.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 146.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 147.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 148.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 149.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 150.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 151.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 152.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 153.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 154.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 155.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 156.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 157.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 158.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 159.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 160.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 161.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 162.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 163.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 1.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 2.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 3.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 4.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 5.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 6.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 7.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 8.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 9.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 10.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 11.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 12.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 13.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 14.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 15.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 16.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 17.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 18.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 19.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 20.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 21.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 22.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 23.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 24.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 25.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 26.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 27.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 28.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 29.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 30.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 81.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 82.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 88.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 89.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 90.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 91.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 92.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 96.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 97.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 119.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 120.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 121.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 122.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 297.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 298.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 299.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 300.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 301.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 302.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 303.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 304.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 305.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 306.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 307.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 308.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 309.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 310.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 311.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 312.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 313.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 372.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 373.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 374.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 375.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 376.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 377.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 378.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 379.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 380.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 381.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 382.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 383.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 384.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 385.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 386.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 387.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 388.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 389.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 390.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 314.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 315.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 316.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 317.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 318.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 319.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 320.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 321.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 322.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 323.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 324.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 325.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 326.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 327.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 328.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 329.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 330.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 331.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 332.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 333.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 334.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 335.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 336.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 337.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 338.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 339.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 340.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 341.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 342.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 343.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 344.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 345.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 346.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 347.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 348.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 349.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 350.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 351.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 352.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 353.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 354.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 355.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 356.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 357.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 358.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 359.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 360.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 361.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 362.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 363.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 364.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 365.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 366.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 367.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 368.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 369.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 370.

In another aspect, provided herein is an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 371.

In another aspect, provided herein is a heterologous construct comprising the engineered macrophage-specific promoter system of any one of the above embodiments; or the engineered macrophage-specific promoter of any one of the above embodiments operably linked to a polynucleotide comprising a nucleotide sequence encoding a polypeptide.

In some embodiments, the polypeptide comprises at least one effector molecule. In some embodiments, the polypeptide comprises a first effector molecule and a second effector molecule.

In some embodiments, the polynucleotide comprises a nucleotide sequence encoding the first effector molecule, a linker nucleotide sequence, and a nucleotide sequence encoding the second effector.

In some embodiments, the linker nucleotide sequence encodes one or more 2A ribosome skipping elements. In some embodiments, the one or more 2A ribosome skipping elements comprise elements that are each selected from the group consisting of: P2A, T2A, E2A, and F2A.

In some embodiments, the effector molecule is selected from a therapeutic class, wherein the therapeutic class is selected from the group consisting of: a cytokine, a chemokine, a homing molecule, a growth factor, a polynucleotide molecule, a co-activation molecule, a tumor microenvironment modifier, a receptor, a ligand, a transcription factor, an antibody, a peptide, and an enzyme.

In some embodiments, the transcription factor is a master regulator. In some embodiments, the transcription factor is a master regulator of polarization to an M1 macrophage. In some embodiments, the transcription factor is IRF7 or a derivative thereof, or p65/RelA or a derivative thereof. In some embodiments, the transcription factor is IRF7 or a derivative thereof, optionally wherein the transcription factor comprises an amino acid sequence having at least 75%, at least 80%, at least 85%, at least 90%, at least 95%, at least 96%, at least 97%, at least 98%, at least 99% or 100% sequence identity to SEQ ID NO: 401, or the amino acid sequence of the transcription factor is SEQ ID NO: 401. In some embodiments, the transcription factor is p65/RelA or a derivative thereof, optionally wherein the transcription factor comprises an amino acid sequence having at least 75%, at least 80%, at least 85%, at least 90%, at least 95%, at least 96%, at least 97%, at least 98%, at least 99% or 100% sequence identity to SEQ ID NO: 403, or the amino acid sequence of the transcription factor is SEQ ID NO: 403.

In some embodiments, the transcription factor is a master regulator of polarization to an M2 macrophage.

In some embodiments, the at least one effector molecule or each effector molecule comprises a cytokine.

In some embodiments, the cytokine is modified to comprise a membrane tethering domain. In some embodiments, the membrane tethering domain is or comprises a transmembrane-intracellular domain and/or transmembrane domain of a protein selected from: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, and BTLA, or a functional portion thereof.

In some embodiments, the membrane tethering domain is or comprises a transmembrane domain of B7-1 protein, or a functional portion thereof.

In some embodiments, the cytokine is IFNgamma.

In some embodiments, the cytokine and the tethering domain are linked by a linker.

In some embodiments, the cytokine is selected from the group consisting of: IL-1alpha, IL1-beta, IL2, IL4, IL6, IL7, IL10, IL12, an IL12p70 fusion protein, IL-12p40, IL-12p35, IL13, IL15, IL17A, IL18, IL21, IL22, Type I interferons, Interferon-gamma, GM-CSF, TGF-beta, M-CSF, and TNF-alpha. In some embodiments, the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, the cytokine is a master regulator of polarization to an M1 macrophage. In some embodiments, the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof. In some embodiments, the cytokine is IFN-7 or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 395, or the amino acid sequence of the cytokine is SEQ ID NO: 395. In some embodiments, the cytokine is TNF-α or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 397, or the amino acid sequence of the cytokine is SEQ ID NO: 397. In some embodiments, the cytokine is IL-12, an IL12p70 fusion protein, or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 399, or the amino acid sequence of the transcription factor is SEQ ID NO: 399.

In some embodiments, the cytokine is a master regulator of polarization to an M2 macrophage. In some embodiments, the cytokine is IL-10, IL-4, IL-13, IL-21, TGF-beta, M-CSF, or a derivative thereof. In some embodiments, the cytokine is IL-10 or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 405, or the amino acid sequence of the transcription factor is SEQ ID NO: 405. In some embodiments, the cytokine is IL-4 or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 407, or the amino acid sequence of the transcription factor is SEQ ID NO: 407.

In some embodiments, the at least one effector molecule or each effector molecule comprises a chemokine. In some embodiments, the chemokine is selected from the group consisting of: CCL21a, CXCL10, CXCL11, CXCL13, a CXCL10-CXCL11 fusion protein, CCL19, CXCL9, and XCL1.

In some embodiments, the at least one effector molecule or each effector molecule comprises a homing molecule. In some embodiments, the homing molecule is selected from the group consisting of: anti-integrin alpha4, beta7; anti-MAdCAM; CCR9; CXCR4; SDF1; MMP-2; CXCR1; CXCR7; CCR2; CCR4; and GPR15.

In some embodiments, the at least one effector molecule or each effector molecule comprises a growth factor. In some embodiments, the growth factor is selected from the group consisting of: FLT3L and GM-CSF.

In some embodiments, the at least one effector molecule or each effector molecule comprises a co-activation molecule. In some embodiments, the co-activation molecule is selected from the group consisting of: c-Jun, 4-1BBL and CD40L.

In some embodiments, the at least one effector molecule or each effector molecule comprises a tumor microenvironment modifier. In some embodiments, the tumor microenvironment modifier is selected from the group consisting of: an adenosine deaminase, a TGFbeta inhibitor, an immune checkpoint inhibitor, a VEGF inhibitor, and an HPGE2.

In some embodiments, each of the first effector molecule and the second effector molecule are from separate therapeutic classes. In some embodiments, each effector molecule is a human-derived effector molecule.

Also provided herein is a heterologous construct for inducing a macrophage to transition from an M1 state to an M2 state, comprising: either (i) the regulatory element derived from a promoter of a gene that is more highly expressed in M1 macrophage compared to M2 or M0 macrophages, or (ii) an engineered macrophage-specific promoter comprising an ablation of at least one nucleotide motif, wherein the ablation increases specific activity of the engineered macrophage-specific promoter in M1 macrophages, as compared to activity of a corresponding macrophage-specific promoter lacking the ablation in M1 macrophages; or (iii) an engineered macrophage-specific promoter comprising at least one regulatory element, wherein the regulatory element exhibits greater activity in an M1 macrophage compared to an M2 or M0 macrophage; and (b) a heterologous payload encoding a master regulator of polarization to an M2 macrophage, wherein the regulatory element or engineered macrophage-specific promoter of (a) is operably linked to the heterologous payload and configured to induce expression of the heterologous payload. In some embodiments, the master regulator of polarization to an M2 macrophage is IL-10, IL-4, IL-13, IL-21, TGF-beta, M-CSF, or a derivative thereof. In some embodiments, the master regulator of polarization to an M2 macrophage is IL-10. In some embodiments, (a) is a regulatory element derived from a CCL19 promoter, optionally comprising the nucleotide sequence of SEQ ID NO: 132. In some embodiments, the M2 state is an M2c state, an M2a state, or an M2b state.

Also provided herein is a heterologous construct for stabilizing a macrophage in an M1 polarization state, comprising: (a) either (i) the regulatory element derived from a promoter of a gene that is more highly expressed in M1 macrophage compared to M2 or M0 macrophages, or (ii) an engineered macrophage-specific promoter comprising an ablation of at least one nucleotide motif, wherein the ablation increases specific activity of the engineered macrophage-specific promoter in M1 macrophages, as compared to activity of a corresponding macrophage-specific promoter lacking the ablation in M1 macrophages; or (iii) an engineered macrophage-specific promoter comprising at least one regulatory element, wherein the regulatory element exhibits greater activity in an M1 macrophage compared to an M2 or M0 macrophage; and (b) a heterologous payload encoding a master regulator of polarization to an M1 macrophage, wherein the regulatory element or engineered macrophage-specific promoter of (a) is operably linked to the heterologous payload and configured to induce expression of the heterologous payload. In some embodiments, the master regulator of polarization to an M1 macrophage is a cytokine. In some embodiments, the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof. In some embodiments, the cytokine is IFN-γ or a derivative thereof. In some embodiments, the master regulator of polarization to an M1 macrophage is a transcription factor selected from IRF7 or a derivative thereof, or p65/RelA or a derivative thereof. In some embodiments, (a) is a regulatory element derived from a UBD1 promoter, an IDO1 promoter, or a CCL19 promoter. In some embodiments, (a) is a regulatory element derived from a UBD1 promoter, optionally wherein the regulatory element derived from the UBD1 promoter comprises the sequence of SEQ ID NO: 137. In some embodiments, (a) is a regulatory element derived from an IDO1 promoter, optionally wherein the regulatory element derived from the IDO1 promoter comprises the sequence of SEQ ID NO: 136: In some embodiments, (a) is a regulatory element derived from a CCL19 promoter, optionally wherein the regulatory element derived from the CCL19 promoter comprises the sequence of SEQ ID NO: 123 or 125.

Also provided herein is a heterologous construct for inducing a macrophage to transition from an M2 state to an M1 state, comprising: (a) either (i) the regulatory element derived from a promoter of a gene that is more highly expressed in M2 macrophage compared to M1 or M0 macrophages, or (ii) an engineered macrophage-specific promoter comprising an ablation of at least one nucleotide motif, wherein the ablation increases specific activity of the engineered macrophage-specific promoter in M2 macrophages, as compared to activity of a corresponding macrophage-specific promoter lacking the ablation in M2 macrophages; or (iii) an engineered macrophage-specific promoter comprising at least one regulatory element, wherein the regulatory element exhibits greater activity in an M2 macrophage compared to an M1 or M0 macrophage; and (b) a heterologous payload encoding a master regulator of polarization to an M1 macrophage, wherein the regulatory element or engineered macrophage-specific promoter of (a) is operably linked to the heterologous payload and configured to induce expression of the heterologous payload. In some embodiments, the master regulator of polarization to an M1 macrophage is a cytokine. In some embodiments, the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof. In some embodiments, the master regulator of polarization to an M1 macrophage is a transcription factor selected from IRF7 or a derivative thereof, or p65/RelA or a derivative thereof. In some embodiments, the master regulator of polarization to an M1 macrophage is IRF7 or a derivative thereof. In some embodiments, the derivative of IRF7 comprises IRF7 operably linked to a degron domain. In some embodiments, the degron domain is selected from: a PEST domain, HCV NS4 degron, GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 190) phospho-dependent degron, an Siah binding motif, an SPOP SBC docking motif, a PCNA binding PIP box, and derivatives thereof. In some embodiments, the degron domain is a PEST domain, optionally wherein the PEST comprises the amino acid sequence SEQ ID NO: 501 or a derivative thereof. In some embodiments, the engineered macrophage specific promoter comprises a regulatory element selected from: (i) a nucleotide sequence having at least 75%, at least 80%, at least 85%, at least 90%, at least 95%, at least 96%, at least 97%, at least 98%, at least 99% or 100% sequence identity to SEQ ID NO: 420; and (ii) a nucleotide sequence having at least 75%, at least 80%, at least 85%, at least 90%, at least 95%, at least 96%, at least 97%, at least 98%, at least 99% or 100% sequence identity to SEQ ID NO: 427. In some embodiments, the engineered macrophage specific promoter comprises a regulatory element having at least 95% sequence identity to SEQ ID NO: 420, optionally having 100% sequence identity to SEQ ID NO: 420. In some embodiments, the regulatory element is operably linked to a minTK minimal promoter or SCP3 minimal promoter. In some embodiments, the engineered macrophage specific promoter comprises a regulatory element having at least 95% sequence identity to SEQ ID NO: 427, optionally having 100% sequence identity to SEQ ID NO: 427. In some embodiments, the regulatory element is operably linked to a minCMV promoter.

Also provided herein is a heterologous construct for stabilizing a macrophage in an M2 polarization state, comprising: (a) either (i) the regulatory element derived from a promoter of a gene that is more highly expressed in M2 macrophage compared to M1 or M0 macrophages, or (ii) an engineered macrophage-specific promoter comprising an ablation of at least one nucleotide motif, wherein the ablation increases specific activity of the engineered macrophage-specific promoter in M2 macrophages, as compared to activity of a corresponding macrophage-specific promoter lacking the ablation in M2 macrophages; or (iii) an engineered macrophage-specific promoter comprising at least one regulatory element, wherein the regulatory element exhibits greater activity in an M2 macrophage compared to an M1 or M0 macrophage; and (b) a heterologous payload encoding a master regulator of polarization to an M2 macrophage, wherein the regulatory element or engineered macrophage-specific promoter of (a) is operably linked to the heterologous payload and configured to induce expression of the heterologous payload. In some embodiments, the master regulator of polarization to an M2 macrophage is IL-10, IL-4, IL-13, IL-21, TGF-beta, M-CSF, or a derivative thereof. In some embodiments, the M2 state is an M2c state, an M2a state, or an M2b state.

In another aspect, provided herein is a vector comprising the heterologous construct according to any one of the above embodiments.

In another aspect, provided herein is a dual expression vector comprising the heterologous construct according to any one of the above embodiments and a second construct comprising a nucleotide sequence encoding an activating immune receptor.

In another aspect, provided herein is an immunoresponsive cell comprising the heterologous construct according to any one of the above embodiments, the vector according to the above embodiment, or the dual expression vector according to the above embodiment. In some embodiments, the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

In some embodiments, the immunoresponsive cell is a macrophage. In some embodiments, the macrophage is a tumor-resident macrophage. In some embodiments, the immunoresponsive cell expresses an activating immune receptor. In some embodiments, the activating immune receptor comprises an antigen recognizing receptor. In some embodiments, the immunoresponsive cell is autologous. In some embodiments, the immunoresponsive cell is allogeneic.

In another aspect, provided herein is a pharmaceutical composition comprising the vector of the above embodiment, the dual expression vector according to the above embodiment, or the immunoresponsive cell according to any one of the above embodiments, and a pharmaceutically acceptable carrier, pharmaceutically acceptable excipient, or a combination thereof.

In another aspect, provided herein is a method of increasing expression of a target gene, the method comprising use of the engineered macrophage-specific promoter of any one of the above embodiments, the vector of the above embodiment, or the dual expression vector according the above embodiment to increase expression of the target gene. In some embodiments, the target gene is an immunomodulatory gene.

In another aspect, provided herein is a method of treating a subject in need thereof, the method comprising administering a therapeutically effective dose of the vector of the above embodiment, the dual expression vector according to the above embodiment, the immunoresponsive cell according to any one of the above embodiments, or the pharmaceutical composition according to the above embodiment.

In another aspect, provided herein is a kit for treating and/or preventing a disease or disorder, comprising the immunoresponsive cell according to any one of the above embodiments.

In another aspect, provided herein is a kit for treating and/or preventing a tumor, comprising the immunoresponsive cell according to any one of the above embodiments. In some embodiments, the kit further comprises written instructions for using the immunoresponsive cell for treating and/or preventing a tumor in a subject.

In another aspect, provided herein is a kit for treating and/or preventing a tumor, comprising the pharmaceutical composition according to the above embodiment.

In another aspect, provided herein is a kit for treating and/or preventing a disease or disorder, comprising the pharmaceutical composition according to the above embodiment.

In some embodiments, the kit of any one of the above aspects further comprises written instructions for using the pharmaceutical composition for treating and/or preventing a tumor in a subject.

The present disclosure further provides, an engineered macrophage-specific promoter system comprising: a regulatory element, wherein the regulatory element is derived from a promoter of a gene selected from the group consisting of CCL19, CCR7, CXCL11, GBP5, IDO1, UBD, and UNQ6494.1; and a heterologous payload, wherein the regulatory element exhibits greater activity in an M1 macrophage compared to an M2 or M0 macrophage. In some embodiments, the regulatory element is or comprises an enhancer region that is derived from a promoter of a gene that is more highly expressed in M1 macrophage compared to M2 or M0 macrophages. In some embodiments, the heterologous payload is selected from the group consisting of transcriptions factors, cytokines, receptors, enzymes, chemokines, antibodies, fragments of antibodies, miRNAs, and shRNAs. In some embodiments, the M2 macrophages are selected from the group consisting of M2a macrophages, M2b macrophages, and M2c macrophages. In some embodiments, the regulatory element comprises a sequence having at least 75%, at least 80%, at least 85%, at least 90%, at least 95%, at least 96%, at least 97%, at least 98%, at least 99% or 100% sequence identity to a nucleotide sequence selected from the group consisting of SEQ ID NO: 132-138.

The present disclosure provides, in some embodiments, an engineered macrophage-specific promoter system comprising: a regulatory element, wherein the regulatory element is or comprises an enhancer region that is derived from a promoter of a gene that is more highly expressed in M1 macrophage compared to M2 or M0 macrophages; and a heterologous payload, wherein the regulatory element exhibits greater activity in an M1 macrophage compared to an M2 or M0 macrophage. In some embodiments, the regulatory element is derived from a promoter of a gene selected from the group consisting of CCL19, CCR7, CXCL11, GBP5, IDO1, UBD, and UNQ6494.1. In some embodiments, the heterologous payload is selected from the group consisting of transcriptions factors, cytokines, receptors, enzymes, chemokines, antibodies, fragments of antibodies, miRNAs, and shRNAs. In some embodiments, the M2 macrophages are selected from the group consisting of M2a macrophages, M2b macrophages, and M2c macrophages. In some embodiments, the regulatory element comprises a sequence having at least 75%, at least 80%, at least 85%, at least 90%, at least 95%, at least 96%, at least 97%, at least 98%, at least 99% or 100% sequence identity to a nucleotide sequence selected from the group consisting of SEQ ID NO: 132-138.

The present disclosure also provides an engineered macrophage-specific promoter system comprising: a regulatory element, wherein the regulatory element is derived from a promoter of a gene selected from the group consisting of CD28, SOCS3, PLXDC1, IL7R ZNF704, LNCAROD, MRC1, and ID3; and a heterologous payload, wherein the regulatory element exhibits greater activity in an M2 macrophage compared to an M1 or M0 macrophage. In some embodiments, the heterologous payload is selected from the group consisting of transcriptions factors, cytokines, receptors, enzymes, chemokines, antibodies, fragments of antibodies, miRNAs, and shRNAs. In some embodiments, the regulatory element is or comprises an enhancer region that is derived from a promoter of a gene that is more highly expressed in M2 macrophage compared to M1 or M0 macrophages, In some embodiments, the M2 macrophages are selected from the group consisting of M2a macrophages, M2b macrophages, and M2c macrophages. In some embodiments, the regulatory element comprises a sequence having at least 75%, at least 80%, at least 85%, at least 90%, at least 95%, at least 96%, at least 97%, at least 98%, at least 99% or 100% sequence identity to a nucleotide sequence selected from the group consisting of SEQ ID NO: 139-141, 392, 393, and 414-419.

The present disclosure also provides, in some embodiments, an engineered macrophage-specific promoter system comprising: a regulatory element, wherein the regulatory element is or comprises an enhancer region that is derived from a promoter of a gene that is more highly expressed in M2 macrophage compared to M1 or M0 macrophages; and a heterologous payload, wherein the regulatory element exhibits greater activity in an M2 macrophage compared to an M1 or M0 macrophage. In some embodiments, the heterologous payload is selected from the group consisting of transcriptions factors, cytokines, receptors, enzymes, chemokines, antibodies, fragments of antibodies, miRNAs, and shRNAs. In some embodiments, the regulatory element is derived from a promoter of a gene selected from the group consisting of CD28, SOCS3, PLXDC1, IL7R ZNF704, LNCAROD, MRC1, and ID3. In some embodiments, the M2 macrophages (e.g., those used as a comparison) are selected from the group consisting of M2a macrophages, M2b macrophages, and M2c macrophages. In some embodiments, the regulatory element comprises a sequence having at least 75%, at least 80%, at least 85%, at least 90%, at least 95%, at least 96%, at least 97%, at least 98%, at least 99% or 100% sequence identity to a nucleotide sequence selected from the group consisting of SEQ ID NO: 139-141, 392, 393, and 414-419.

The present disclosure provides an engineered macrophage-specific promoter comprising an ablation of at least one nucleotide motif, wherein the ablation increases specific activity of the engineered macrophage-specific promoter in M1 macrophages, as compared to activity of a corresponding macrophage-specific promoter lacking the ablation in M1 macrophages. In some embodiments, the corresponding macrophage-specific promoter lacking the ablation in M1 macrophages is a wildtype macrophage promoter, and wherein the wildtype macrophage promoter comprises a sequence selected from the group consisting of SEQ ID NOs: 132-138, wherein the engineered macrophage-specific promoter comprises: a motif within the nucleotide sequence of SEQ ID NO: 132, wherein the motif comprises a sequence selected from the group consisting of: position 63 to position 73 of SEQ ID NO: 132, position 80 to position 102 of SEQ ID NO: 132, position 141 to position 162 of SEQ ID NO: 132, position 212 to position 222 of SEQ ID NO: 132, position 229 to position 251 of SEQ ID NO: 132, position 307 to position 361 of SEQ ID NO: 132, position 365 to position 376 of SEQ ID NO: 132, position 559 to position 571 of SEQ ID NO: 132, position 617 to position 633 of SEQ ID NO: 132, position 782 to position 799 of SEQ ID NO: 132, position 852 to position 871 of SEQ ID NO: 132, position 886 to position 920 of SEQ ID NO: 132, position 933 to position 959 of SEQ ID NO: 132, position 1002 to position 1028 of SEQ ID NO: 132, position 1032 to position 1045 of SEQ ID NO: 132, position 1064 to position 1087 of SEQ ID NO: 132, position 1169 to position 1192 of SEQ ID NO: 132, position 1212 to position 1232 of SEQ ID NO: 132, position 1257 to position 1275 of SEQ ID NO: 132, position 1310 to position 1333 of SEQ ID NO: 132, position 1381 to position 1434 of SEQ ID NO: 132, position 1698 to position 1753 of SEQ ID NO: 132, position 1783 to position 1826 of SEQ ID NO: 132, position 1909 to position 1927 of SEQ ID NO: 132, position 1946 to position 1961 of SEQ ID NO: 132; and/or a motif within the nucleotide sequence of SEQ ID NO: 136, wherein the motif comprises a sequence selected from the group consisting of: to position 133 to position 144 of SEQ ID NO: 136, position 200 to 217 of SEQ ID NO: 136, position 225 to position 247 of SEQ ID NO: 136, position 303 to position 325 of SEQ ID NO: 136, position 332 to position 342 of SEQ ID NO: 136, position 391 to position 413 of SEQ ID NO: 136, position 423 to position 460 of SEQ ID NO: 136, position 467 to position 477 of SEQ ID NO: 136, position 693 to position 717 of SEQ ID NO: 136, position 738 to position 761 of SEQ ID NO: 136, position 838 to position 861 of SEQ ID NO: 136, position 1229 to position 1246 of SEQ ID NO: 136, position 1286 to position 1309 of SEQ ID NO: 136, position 1413 to position 1431 of SEQ ID NO: 136, position 1456 to position 1473 of SEQ ID NO: 136, to position 1530 to position 1544 of SEQ ID NO: 136, position 1577 to position 1590 of SEQ ID NO: 136, position 1816 to position 1836 of SEQ ID NO: 136, position 1852 to position 1872 of SEQ ID NO: 136, and to position 1876 to position to position 1896 of SEQ ID NO: 136; and/or a motif within the nucleotide sequence of SEQ ID NO: 137, wherein the motif comprises a sequence selected from the group consisting of: to position 43 to position 60, position 107 to position 120 of SEQ ID NO: 137, position 210 to position 230 of SEQ ID NO: 137, position 345 to position 407 of SEQ ID NO: 137, position 427 to position 457 of SEQ ID NO: 137, position 468 to position 484 of SEQ ID NO: 137, position 560 to position 582, position 730 to position 746 of SEQ ID NO: 137, position 809 to position 820 of SEQ ID NO: 137, position 827 to position 837 of SEQ ID NO: 137, position 858 to position 878 of SEQ ID NO: 137, position 1291 to position 1302 of SEQ ID NO: 137, position 1321 to position 1341 of SEQ ID NO: 137, position 1435 to position 1463 of SEQ ID NO: 137, position 1530 to position 1541 of SEQ ID NO: 137, position 1707 to position 1718 of SEQ ID NO: 137, position 1834 to position 1863 of SEQ ID NO: 137, position 1870 to position 1882 of SEQ ID NO: 137, and to position 1913 to position 1929 of SEQ ID NO: 137. In some embodiments, the ablation comprises a substitution or deletion of one or more nucleotides of the at least one nucleotide motif.

The present disclosure provides an engineered macrophage-specific promoter comprising at least one regulatory element, wherein the regulatory element exhibits greater activity in an M1 macrophage compared to an M2 or M0 macrophage or exhibits greater activity in an M2 macrophage compared to an M1 or M0 macrophage. In some embodiments, the engineered macrophage-specific promoter comprises at least 2, at least 3, at least 4, or at least 5 regulatory elements. In some embodiments, each of the regulatory elements are the same or different. In some embodiments, the M2 macrophages are selected from the group consisting of M2a macrophages, M2b macrophages, and M2c macrophages.

In some embodiments, the at least one regulatory element comprises a nucleotide sequence selected from: a nucleotide sequence having at least 75%, at least 80%, at least 85%, at least 90%, at least 95%, at least 96%, at least 97%, at least 98%, at least 99% or 100% sequence identity to SEQ ID NO: 297-313; a nucleotide sequence having at least 75%, at least 80%, at least 85%, at least 90%, at least 95%, at least 96%, at least 97%, at least 98%, at least 99% or 100% sequence identity to SEQ ID NO: 372-390; a nucleotide sequence having at least 75%, at least 80%, at least 85%, at least 90%, at least 95%, at least 96%, at least 97%, at least 98%, at least 99% or 100% sequence identity to SEQ ID NO: 440-443, a nucleotide sequence having at least 75%, at least 80%, at least 85%, at least 90%, at least 95%, at least 96%, at least 97%, at least 98%, at least 99% or 100% sequence identity to SEQ ID NOs: 314-371, a nucleotide sequence having at least 75%, at least 80%, at least 85%, at least 90%, at least 95%, at least 96%, at least 97%, at least 98%, at least 99% or 100% sequence identity to SEQ ID NO: 420-439.

In some embodiments, a provided engineered macrophage-specific promoter further comprises a minimal promoter operably linked to the engineered macrophage-specific promoter. In some embodiments, the minimal promoter is derived from a promoter selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, inducer molecule responsive promoters, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, hUBIb, and tandem repeats thereof.

The present disclosure provides an engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 1-29, 81-82, 88-97, 119-122, 132-138, 142-163, 97-313, 139-141, 314-371, 390, 392-393, and 420-443. In some embodiments, the regulatory element or the engineered macrophage-specific promoter is operably linked to a minimal promoter. In some embodiments, the minimal promoter comprises a sequence of a promoter selected from minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, SCP3, YB-SCP3, inducer molecule responsive promoters, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, hUBIb, and tandem repeats thereof. In some embodiments, the engineered macrophage-specific promoter system further comprises a translation initiator site. In some embodiments, the translation initiator site is or comprises a Kozak sequence.

In some embodiments, the regulatory element or the engineered macrophage-specific promoter comprises: a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256; and does not comprise at least one repressive element selected from: SEQ ID NO: 226, SEQ ID NO: 234, SEQ ID NO: 236, SEQ ID NO: 238, SEQ ID NO: 246, and SEQ ID NO: 252. In some embodiments, the regulatory element or the engineered macrophage-specific promoter further comprises a sixth transcriptional activating element as set forth in SEQ ID NO: 224 and/or a seventh transcriptional activating element as set forth in SEQ ID NO: 258. In some embodiments, the regulatory element or the engineered macrophage-specific promoter further do not comprise SEQ ID NO: 228, SEQ ID NO: 230, SEQ ID NO: 232, SEQ ID NO: 242, SEQ ID NO:244, SEQ ID NO: 248, and SEQ ID NO: 250. In some embodiments, the regulatory element or the engineered macrophage-specific promoter does not comprise the repressive elements as set forth in SEQ ID NO: 226, SEQ ID NO: 236, SEQ ID NO: 238, SEQ ID NO: 246, and SEQ ID NO: 252. In some embodiments, the regulatory element or the engineered macrophage-specific promoter comprises: a sequence as set forth in GTTAAGTGGCTAGGGATAACATTGAGGCACTAAAGCATTATTGGTTCTGCAGTCA AGGGTAGGATAGATTGTTTTTTTTTTTTT (SEQ ID NO: 482), and a sequence as set forth in TTTGTGGTTTTATTGGTTTTCATATTACAAACAAAGAAACTAGAAAATGAAACCA TTCCAAAAGTGGAAGTAATTTCTCA (SEQ ID NO: 483), or a sequence as set forth in GCTCTTCTAAAAATATGCGAAATGAGGTTTTTAGGGAGGTGTAGGTATGGCTGAA GAAAATCAAGGTGAATGAAGACAAGATCAATTGAGAATGTAGTTTCAGAAATAG CAAAGAAGCCAAAGTTTGAGGAAGTTAAGTGGCTAGGGATAACATTGAGGCACT AAAGCATTATTGGTTCTGCAGTCAAGGGTAGGATAGATTGTTTTTTTTTTTTTTGA GACGGAGTCTCACTCTGCTGCCCAGGC (SEQ ID NO: 484), a sequence as set forth in ATTTTGGTTTCAGTTTTCCTTAC (SEQ ID NO: 240), and a sequence as set forth in TTTGTGGTTTTATTGGTTTTCATATTACAAACAAAGAAACTAGAAAATGAAACCA TTCCAAAAGTGGAAGTAATTTCTCA (SEQ ID NO: 483); or a first transcriptional activating element as set forth in SEQ ID NO: 268 and a second transcriptional activating element as set forth in SEQ ID NO: 270 and does not comprise at least one repressive element selected from: SEQ ID NO: 260, SEQ ID NO: 262, SEQ ID NO: 264, SEQ ID NO: 266, SEQ ID NO: 272, and SEQ ID NO: 391; or at least one, at least two, at least three, at least four, or at least five tandem repeats of SEQ ID NO: 268 and SEQ ID NO: 270. In some embodiments, the regulatory element or the engineered macrophage-specific promoter further comprises a third transcriptional activating element as set forth in SEQ ID NO: 291 and/or a fourth transcriptional activating element as set forth in: SEQ ID NO: 295. In some embodiments, the regulatory element or the engineered macrophage-specific promoter does not comprise the repressive elements as set forth in SEQ ID NO: 262, SEQ ID NO: 264, SEQ ID NO: 272, and SEQ ID NO: 391. In some embodiments, the regulatory element or the engineered macrophage-specific promoter further does not comprise SEQ ID NO: 260 and/or SEQ ID NO: 266.

The present disclosure provides a heterologous construct comprising any engineered macrophage-specific promoter system as described herein; or any engineered macrophage-specific promoter as described herein. In some embodiments, an engineered macrophage-specific promoter system as described herein; or an engineered macrophage-specific promoter as described herein is operably linked to a heterologous payload. In some embodiments, the heterologous payload is a polynucleotide comprising a nucleotide sequence encoding a polypeptide. In some embodiments, the polypeptide comprises at least one effector molecule. In some embodiments, the polypeptide comprises a first effector molecule and a second effector molecule. In some embodiments, the engineered macrophage specific promoter comprises a regulatory element selected from: a nucleotide sequence having at least 75%, at least 80%, at least 85%, at least 90%, at least 95%, at least 96%, at least 97%, at least 98%, at least 99% or 100% sequence identity to SEQ ID NO: 420; and a nucleotide sequence having at least 75%, at least 80%, at least 85%, at least 90%, at least 95%, at least 96%, at least 97%, at least 98%, at least 99% or 100% sequence identity to SEQ ID NO: 427. In some embodiments, the polynucleotide comprises a nucleotide sequence encoding the first effector molecule, a linker nucleotide sequence, and a nucleotide sequence encoding the second effector. In some embodiments, the linker nucleotide sequence encodes one or more 2A ribosome skipping elements. In some embodiments, the one or more 2A ribosome skipping elements comprise elements that are each selected from the group consisting of: P2A, T2A, E2A, and F2A.

In some embodiments, the at least one effector molecule or each effector molecule is selected from a therapeutic class, wherein the therapeutic class is selected from the group consisting of: a cytokine, a chemokine, a homing molecule, a growth factor, a polynucleotide molecule, a co-activation molecule, a tumor microenvironment modifier, a receptor, a ligand, a transcription factor, an antibody, a peptide, and an enzyme. In some embodiments, the transcription factor is a master regulator. In some embodiments, the transcription factor is a master regulator of polarization to an M1 macrophage. In some embodiments, the transcription factor is IRF7 or a derivative thereof, or p65/RelA or a derivative thereof. In some embodiments, the transcription factor is a master regulator of polarization to an M2 macrophage. In some embodiments, the at least one effector molecule or each effector molecule is or comprises a cytokine, chemokine, homing molecule, growth factor, or a tumor microenvironment modifier. In some embodiments, the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha. In some embodiments, the cytokine is a master regulator of polarization to an M1 macrophage. In some embodiments, the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof. In some embodiments, the cytokine is a master regulator of polarization to an M2 macrophage. In some embodiments, the cytokine is IL-10, IL-4, IL-13, IL-21, TGF-beta, M-CSF, or a derivative thereof. In some embodiments, the chemokine is selected from the group consisting of: CCL21a, CXCL10, CXCL11, CXCL13, a CXCL10-CXCL11 fusion protein, CCL19, CXCL9, and CXCL1. In some embodiments, the homing molecule is selected from the group consisting of: anti-integrin alpha4, beta7; anti-MAdCAM; CCR9; CXCR4; SDF1; MMP-2; CXCR1; CXCR7; CCR2; CCR4; and GPR15. In some embodiments, the growth factor is selected from the group consisting of: FLT3L and GM-CSF. In some embodiments, the co-activation molecule is selected from the group consisting of: c-Jun, 4-1BBL and CD40L. In some embodiments, the tumor microenvironment modifier is selected from the group consisting of: an adenosine deaminase, a TGFbeta inhibitor, an immune checkpoint inhibitor, a VEGF inhibitor, and an HPGE2. In some embodiments, each of the first effector molecule and the second effector molecule are from separate therapeutic classes. In some embodiments, each effector molecule is a human-derived effector molecule. In some embodiments, the cytokine is modified to comprise a membrane tethering domain. In some embodiments, the membrane tethering domain is or comprises a transmembrane-intracellular domain and/or transmembrane domain of a protein selected from: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, and BTLA, or a functional portion thereof. In some embodiments, the master regulator of polarization to an M1 macrophage is IRF7 or a derivative thereof. In some embodiments, the derivative of IRF7 comprises IRF7 operably linked to a degron domain. In some embodiments, the degron domain is selected from: a PEST domain, HCV NS4 degron, GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 190) phospho-dependent degron, an Siah binding motif, an SPOP SBC docking motif, a PCNA binding PIP box, and derivatives thereof. In some embodiments, the degron domain is a PEST domain. In some embodiments, the PEST comprises the amino acid sequence SEQ ID NO: 501 or a derivative thereof.

The present disclosure provides a heterologous construct for inducing a macrophage to transition from an M1 state to an M2 state, comprising: either a regulatory element derived from a promoter of a gene that is more highly expressed in M1 macrophage compared to M2 or M0 macrophages, as provided herein, or an engineered macrophage-specific promoter as provided herein; and a heterologous payload encoding a master regulator of polarization to an M2 macrophage, wherein the regulatory element or engineered macrophage-specific promoter of (a) is operably linked to the heterologous payload and configured to induce expression of the heterologous payload. In some embodiments, the master regulator of polarization to an M2 macrophage is IL-10, IL-4, IL-13, IL-21, TGF-beta, M-CSF, or a derivative thereof. In some embodiments, the master regulator of polarization to an M2 macrophage is IL-10. In some embodiments, the M2 state is an M2c state, an M2a state, or an M2b state. In some embodiments, (a) is a regulatory element derived from a CCL19 promoter. In some embodiments, (a) comprises the nucleotide sequence of SEQ ID NO: 132.

The present disclosure provides a heterologous construct for stabilizing a macrophage in an M1 polarization state, comprising: either a regulatory element derived from a promoter of a gene that is more highly expressed in M1 macrophage compared to M2 or M0 macrophages. In some embodiments, the regulatory element derived from a UBD1 promoter, an IDO1 promoter, or a CCL19 promoter, as described herein, or an engineered macrophage-specific promoter as described herein; and a heterologous payload encoding a master regulator of polarization to an M1 macrophage, wherein the regulatory element or engineered macrophage-specific promoter of (a) is operably linked to the heterologous payload and configured to induce expression of the heterologous payload. In some embodiments, the master regulator of polarization to an M1 macrophage is a cytokine. In some embodiments, the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof. In some embodiments, the master regulator of polarization to an M1 macrophage is a transcription factor selected from IRF7 or a derivative thereof, or p65/RelA or a derivative thereof.

The present disclosure provides a heterologous construct for inducing a macrophage to transition from an M2 state to an M1 state, comprising: either a regulatory element derived from a promoter of a gene that is more highly expressed in M2 macrophage compared to M1 or M0 macrophages, as described herein, or an engineered macrophage-specific promoter as described herein; and a heterologous payload encoding a master regulator of polarization to an M1 macrophage, wherein the regulatory element or engineered macrophage-specific promoter of (a) is operably linked to the heterologous payload and configured to induce expression of the heterologous payload. In some embodiments, the master regulator of polarization to an M1 macrophage is a cytokine. In some embodiments, the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof. In some embodiments, the master regulator of polarization to an M1 macrophage is a transcription factor selected from IRF7 or a derivative thereof, or p65/RelA or a derivative thereof.

The present disclosure provides a heterologous construct for stabilizing a macrophage in an M2 polarization state, comprising: either a regulatory element derived from a promoter of a gene that is more highly expressed in M2 macrophage compared to M1 or M0 macrophages, as described herein, or an engineered macrophage-specific promoter as described herein; and a heterologous payload encoding a master regulator of polarization to an M2 macrophage, wherein the regulatory element or engineered macrophage-specific promoter of (a) is operably linked to the heterologous payload and configured to induce expression of the heterologous payload. In some embodiments, the M2 state is an M2c state, an M2a state, or an M2b state. In some embodiments, the master regulator of polarization to an M2 macrophage is IL-10, IL-4, IL-13, IL-21, TGF-beta, M-CSF, or a derivative thereof.

The present disclosure provides a vector comprising any heterologous construct described herein.

The present disclosure provides a dual expression vector comprising any heterologous construct provided herein and a second construct comprising a nucleotide sequence encoding an activating immune receptor.

The present disclosure provides an immunoresponsive cell comprising any heterologous construct described herein, any vector described herein, or any dual expression vector described herein. IN some embodiments the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In some embodiments, the immunoresponsive cell is a macrophage. In some embodiments, the macrophage is a tumor-resident macrophage. In some embodiments, the immunoresponsive cell is autologous or allogeneic. In some embodiments, the immunoresponsive cell expresses an activating immune receptor. In some embodiments, the activating immune receptor comprises an antigen recognizing receptor.

The present disclosure provides a pharmaceutical composition comprising any vector described herein, any dual expression vector described herein, or any immunoresponsive cell described herein, and a pharmaceutically acceptable carrier, pharmaceutically acceptable excipient, or a combination thereof.

The present disclosure provides a method of increasing expression of a target gene, the method comprising use of any engineered macrophage-specific promoter described herein, any vector described herein, or any dual expression vector described herein, to increase expression of the target gene. In some embodiments, the target gene is an immunomodulatory gene.

The present disclosure provides a method of treating a subject in need thereof, the method comprising administering a therapeutically effective dose of any vector described herein, any dual expression vector described herein, any immunoresponsive cell described herein, or any pharmaceutical composition described herein.

The present disclosure provides a kit for treating and/or preventing a disease or disorder, comprising any immunoresponsive cell described herein or any pharmaceutical composition described herein. In some embodiments, the kit further comprises written instructions for using the immunoresponsive cell for treating and/or preventing a disease or disorder in a subject. In some embodiments, the disease is cancer.

### BRIEF DESCRIPTION OF THE DRAWINGS

The patent or application file contains at least one drawing executed in color. Copies of this patent or patent application with color drawing(s) will be provided by the Office upon request and payment of the necessary fees.

FIG. 1 depicts fluorescence level of a fluorescent reporter regulated by native promoters associated with macrophage phenotype linked to a fluorescent protein reporter. The x-axis shows the reporter expression level from cells transduced with that promoter-reporter pair normalized to cells transduced with no virus (NV). The y-axis shows the activity in M1 polarized cells divided by either M0 (circles) or M2c (squares).

FIG. 2 is a schematic of the engineering of native macrophage promoter sequences and selective ablation of regulatory motifs.

FIG. 3 depicts fluorescence level of a fluorescent reporter regulated by engineered macrophage-specific promoters, comparing M1 macrophage selectivity and M2 macrophage selectivity. Color (grayscale) indicates the reporter expression level from cells transduced with that promoter-reporter pair normalized to cells transduced with no virus (NV).

FIG. 4 depicts fluorescence level of a fluorescent reporter regulated by selected engineered macrophage-specific promoters. Color (grayscale) indicates the reporter expression level from cells transduced with that promoter-reporter pair normalized to cells transduced with no virus (NV). The x-axis shows the expression in M1 polarized cells divided by M0 polarized, and the y-axis shows the expression in M1 polarized cells divided by M2c polarized. SB07683 is a constitutive control; SB06353 is the original native promoter, and SB08123 to SB08126 engineered promoters.

FIG. 5 depicts re-screening of native promoters associated with macrophage phenotype linked to a fluorescent protein reporter using VPX accessory protein during lentiviral packaging for improved transduction efficiency.

FIG. 6 depicts fluorescence of selected native macrophage promoter sequences determined from bulk RNA-seq data, identified from both protein coding and noncoding genes.

FIG. 7 depicts fluorescence level of a fluorescent reporter regulated by of selected native M2 macrophage promoters. The x-axis shows the reporter expression level from cells transduced with that promoter-reporter pair normalized to cells transduced with no virus (NV). The y-axis shows the activity in M2c polarized cells divided by either M0 (circles) or M1 (triangles).

FIG. 8 is a schematic depicting engineered enhancers with length of up to 100 bp and made of arrays of transcription factor (TF) binding sites selected based on motif enrichment analysis.

FIG. 9 is a schematic of the primer binding sites for MPRA based next-generation sequencing analysis of our integrated promoter library constructs. Open arrows indicate primer binding sites used in Next generation sequencing (NGS).

FIG. 10 depicts schematically the general protocol of screening of engineered promoter sequences.

FIGS. 11A-C depicts engineered promoters identified from the promoter library that are selective for M1 vs M0 polarization state (FIG. 11A), M2c vs M0 polarization state (FIG. 11B), and M1 vs M2c polarization state (FIG. 11C).

FIGS. 12A-C depicts heat maps of engineered promoters identified from the promoter library that are selective for M1 vs M0 polarization state (FIG. 12A), M2c vs M0 polarization state (FIG. 12B), M1 vs M2c polarization state (FIG. 12C) and indicates distinct patterns of motif enrichment.

FIG. 13 provides selected hits from the SB07479 targeted M1 library screening.

FIG. 14 depicts macrophage polarization to M1 or M2 states, and phenotype plasticity of macrophages between M1 and M2 states.

FIG. 15 depicts exemplary promoter system designs for keeping macrophages in a stable M2 state or directing M1 macrophages from an M1 phenotype to M2 phenotype.

FIG. 16 depicts exemplary promoter system designs for keeping macrophages in a stable M1 state or directing M2 macrophages from an M2 phenotype to M1 phenotype.

FIG. 17 depicts polarization state selective activity and promoter strength as analyzed by flow cytometry. Color scale indicates the promoter strength (normalized to the EFS constitutive promoter). The x-axis shows M1/M0 state selectivity, and the y-axis shows M1/M2c state selectivity. SB07683 is a constitutive control, SB09385 includes the original native IDO1 promoter sequence (has the same IDO1 promoter sequence as SB05125), and SB09386-SB09405 include the promoter ablation variants of the IDO1 promoter (see Tables 1 and 2).

FIG. 18 depicts polarization state selective activity and promoter strength as analyzed by flow cytometry. Color scale indicates the promoter strength (normalized to the EFS constitutive promoter). The x-axis shows M1/M0 state selectivity, and the y-axis shows M1/M2c state selectivity. SB07683 is a constitutive control, SB09406 includes the original native UBD1 promoter sequence (has the same UBD1 promoter sequence as SB05132), and SB09407 through SB09425 include the ablation variants of the UBD1 native promoter (see Tables 1 and 2).

FIG. 19 depicts polarization state selective activity and promoter strength as analyzed by flow cytometry. Color scale indicates the promoter strength (normalized to the EFS constitutive promoter). The x-axis shows M1/M0 state selectivity, and the y-axis shows M1/M2c state selectivity. SB07683 is a constitutive control.

FIGS. 20A-B depict results from an experiment screening candidate master regulators of M1 state polarization in macrophages. FIG. 20A depicts principal component loadings for quantified variables, and FIG. 20B depicts principal component analysis of the master regulator candidates.

FIG. 21 depicts a schematic of the experimental design for candidate M1 phenotype lock circuit screening.

FIGS. 22A-C depict results from an experiment screening candidate M1 phenotype lock circuits. FIG. 22A depicts principal component loadings for quantified variables. FIG. 22B depicts aggregated phenotypes in the 2-dimensional principal component space of all candidate M1 lock circuits across all polarization conditions. FIG. 22C depicts aggregated phenotypes in the 2-dimensional principal component space of selected candidate M1 lock circuits as well as the positive control.

FIG. 23 depicts polarization state selective activity and promoter strength as analyzed by GFP expression. Color scale indicates the promoter strength as fold change over no virus.

FIG. 24 depicts IL-10 expression induced by the M1 polarization conditions as compared to the M0 polarization condition. M0, M1 Low, M1+(No LPS), and M1++(with LPS) are shown for each time point from left to right, respectively.

FIG. 25 depicts assessment of changes in cell phenotype due to phenotype switch circuit activity.

FIG. 26 depicts results of new M2 state selective promoter screening.

FIG. 27 depicts results of an additional round of new M2 state selective promoter screening.

FIG. 28A and FIG. 28B depict state-selective promoter activity and strength of engineered M2 promoters, derived from ATAC-Seq nominated enhancers.

FIG. 29 depicts state-selective promoter activity and strength of engineered M2 promoters, derived from MPRA library screening.

FIG. 30 depict state-selective promoter activity and strength of engineered M2 promoters, derived from re-engineered ATAC-Seq nominated enhancers.

FIG. 31 depicts promoter activity of Enhancers 1-8 paired with alternative core promoters, minPros 1-6. In each section (separated by dashed vertical lines) the activity of a minPRO paired with enhancers 1-8 is shown from left to right, respectively (e.g., minPRO1 with enhancer 1, minPRO1 with enhancer 2, minPRO1 with enhancer 3, and so forth). Each group of 3 of the same colored-bars represent a single enhancer and minimal promoter pairing that was tested in the M0, M1, and M2c polarization states, respectively (while the X axis of FIG. 31 shows only M0 labeling, promoter activity of each construct is depicted as three lines of the same color, representing activity in the M0, M1, and M2c polarization states, from left to right).

FIG. 32 depicts state-selective promoter activity and strength of select enhancers (selected from Enhancers 1-8) paired with alternative core promoters (selected from minPros 1-6).

FIG. 33 depicts state-selective promoter activity and strength of select enhancers (selected from Enhancers 1-8) paired with alternative core promoters (selected from minPros 1-6).

FIG. 34A depicts functional regions of the IDO1 native promoter sequence that were mapped from the ablation screening experiment of Example 2. Activating elements shown include SB09386, SB09387, SB09396, SB09403, and SB09404. Repressive elements shown include SB09389, SB09393, SB09394, SB09395, SB09399, and SB09402. Non-specific activator elements shown include SB09388 and SB09405. FIG. 34B depicts an exemplary map of select re-engineered IDO1 promoters (construct IDs SB12087, SB12090, and SB12091). Activating elements shown include SB09386, SB09387, SB09396, SB09403, and SB09404. Repressive elements shown include SB09389, SB09393, SB09394, SB09395, SB09399, and SB09402. Non-specific activator elements shown include SB09388 and SB09405. FIG. 34C depicts state-selective promoter activity and strength of select re-engineered IDO1 promoters. FIG. 34D depicts state-selective promoter activity and strength of select re-engineered IDO1 promoters compared to the native IDO1 promoter and single ablation IDO1 promoters. SB12087, SB12090, and SB12091 3^{rd} Gen Pro indicated in the graph.

FIG. 35A depicts functional regions of the UBD1 native promoter sequence that were mapped from the ablation screening experiment of Example 3. Activating elements shown include SB09411, SB09412, SB09423, and SB09425. Leaky elements shown include SB09407, SB09408, SB09409, SB09410, SB09413, and SB09414. FIG. 35B depicts an exemplary map of select re-engineered UBD1 promoters (construct IDs SB12093, SB12094, SB12095, SB12096, SB12097, SB12098, and SB12099). Activating elements shown include SB09411, SB09412, SB09423, and SB09425. Leaky elements shown include SB09407, SB09408, SB09409, SB09410, SB09413, and SB09414. FIG. 35C depicts state-selective promoter activity and strength of select re-engineered UBD1 promoters. FIG. 35D depicts state-selective promoter activity and strength of select re-engineered UBD1 promoters compared to the native UBD1 promoter and single ablation UBD1 promoters.

FIG. 36 depicts expression of M1-associated markers in M0-polarized cells that were previously transduced with constructs expressing soluble IFNg, tethered IFNg, or mCherry under control of constitutive EFS promoter.

FIG. 37 depicts expression of M2-associated markers in M0 and M2c polarized cells that were previously transduced with constructs expressing soluble IFNg, tethered IFNg, or mCherry under control of constitutive EFS promoter.

FIG. 38 depicts aggregated phenotypes in the 2-dimensional principal component space of select candidate M1 lock circuits expressing soluble IFNg across the following three polarization conditions: the M0 “basal” condition, the M1→M0 “repolarized” condition, and M1→M1 “target” condition.

FIG. 39 depicts aggregated phenotypes in the 2-dimensional principal component space of selected candidate M1 lock circuits expressing soluble IFNg across the following 3 polarization conditions: the M2c “polarized” condition, the M1→M2c “trans-polarized” condition, and M1→M1 “target” condition.

FIG. 40 depicts aggregated phenotypes in the 2-dimensional principal component space of selected candidate M1 lock circuits expressing membrane-tethered IFNg across the following 3 polarization conditions: the M0 “basal” condition, the M1→M0 “re-polarized” condition, and M1→M1 “target” condition.

FIG. 41 depicts aggregated phenotypes in the 2-dimensional principal component space of selected candidate M1 lock circuits expressing membrane-tethered IFNg across the following 3 polarization conditions: the M2c “polarized” condition, the M1→M2c “trans-polarized” condition, and M1→M1 “target” condition.

FIG. 42 depicts performance of SB11463 on TNFalpha, GROalpha, and IL-6.

FIG. 43 depicts performance of SB11503 on TNFalpha, GROalpha, and IL6.

FIG. 44A and FIG. 44B depict soluble and membrane tethered IFNg payload gene circuit performance in locking TNFalpha production under repolarization conditions (M1→M0).

FIG. 45A and FIG. 45B depict soluble and membrane tethered IFNg payload gene circuit performance in locking TNFalpha production under transpolarization conditions (M1→M2c).

FIGS. 46A-46D depict results of an experiment testing various M2→M1 phenotype switch constructs.

### DETAILED DESCRIPTION

### Definitions

Terms used in the claims and specification are defined as set forth below unless otherwise specified.

By the term “macrophage-specific promoter,” it is meant a promoter that is determined to have higher activity in one macrophage polarization state over another macrophage polarization state. Macrophages can transition between different polarization states, such as the M1 macrophage or M2 macrophage polarization state. For example, in some embodiments, a macrophage-specific promoter has higher activity in a macrophage in the M1 polarization state compared to a macrophage in the M2 polarization state. In some embodiments, polarization of M2 macrophages can transition M2 macrophages into different M2 macrophage subtypes depending on the stimulatory cues. These can include, but are not limited to, M2a, M2b, or M2c subtypes.

The term “ameliorating” refers to any therapeutically beneficial result in the treatment of a disease state, e.g., a cancer disease state, including prophylaxis, lessening in the severity or progression, remission, or cure thereof.

The term “in vitro” refers to processes that occur in a living cell growing separate from a living organism, e.g., growing in tissue culture.

The term “in vivo” refers to processes that occur in a living organism.

The term “mammal” as used herein includes both humans and non-human animals, and includes but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are set. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

### Polarization-Specific Promoters and Ablation Variants

In one aspect, described herein are polarization state-specific promoters (e.g., M1, M2, M0) for use in engineered macrophages.

As used herein, an “ablation” refers to a deletion, using any means of nucleotide deletion as known in the art (e.g., molecular cloning, CRISPR, etc.). Ablation may further comprise replacement of a segment of the nucleotide sequence with a transcriptionally inert segment of the same length. In some embodiments, ablation of the nucleotide motif increases activity and/or selectivity of the promoter (e.g., transcriptional levels downstream of the promoter following stimulation), wherein the increased activity and/or selectivity is relative to the promoter lacking such ablation.

Methods of quantifying transcriptional levels are known in the art and include, for example and without limitation, mRNA analysis by reverse-transcriptase quantitative polymerase chain reaction (RT-qPCR), fluorescent reporters, colorimetric reporters, etc. In some embodiments, the ablation increases inducibility by at least 0.5-fold, at least 1-fold, at least 2-fold, at least 3-fold, at least 4-fold, at least 5-fold, at least 6-fold, at least 7-fold, or at least 8-fold. It is contemplated herein that changes in inducibility of the engineered promoter may depend on the test system, e.g., the cell type comprising the promoter.

In some embodiments, the ablation comprises a substitution of a second nucleotide motif at the site of ablation (i.e., a second nucleotide motif sequence is inserted at a site of an ablation). In some embodiments, introduction of the second nucleotide motif in the ablation site does not introduce new regulatory sites in the engineered promoter (e.g., transcription factor binding sites). Such engineered promoters, as provided herein, comprising a deletion or a substitution of a nucleotide motif at the site of ablation are referred to herein as “ablation variants.”

In some embodiments, any of the promoters described herein may further comprise a translation initiator site at a 3′ end of the promoter, e.g., a consensus Kozak sequence. An exemplary consensus Kozak sequence may be or may include the nucleotide sequence GCCACC. In some embodiments, a translation initiator site (e.g., a Kozak sequence) comprises a flanking spacer sequence at the 5′ and/or 3′ end. In some embodiments, a spacer sequence comprises a nucleotide sequence of ACGCGTACCGGTGTC (SEQ ID NO: 496). In some embodiments, a translation initiator site with a flanking spacer sequence comprises a nucleotide sequence of ACGCGTACCGGTGTCGCCACC (SEQ ID NO: 497). In some embodiments, a promoter described herein does not comprise a translation initiator site.

Sequences of exemplary native and engineered promoters are provided in Table 1 below. Sequences of the wildtype ablation motif and the second nucleotide motif used for substitution of ablation variants, where applicable, are provided in Table 2.

TABLE 1
DNA sequences of exemplary native and engineered promoters

| PROMOTER | | | |
|---|---|---|---|
| ID | NAME | SEQUENCE | SEQ ID NO |
| SB05116/ | CCL19 | GATGAATTAATGAACAGATACCTGAATAAAAGGATAGAAGGC | 132 |
| SB06353 | | CAGGCACGGTGGCTCACGCCTGTAATCCCAACACTTTGGGAAG | |
| | | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB05117 | CCR7 | CAATTGAACCTCCACATTTAAAACTTTGCGCTTCTTTTGCCAGA | 133 |
| | | GATTCAGGTTTTAAAAAACAAAAAGCCTTTGTGCTTCAAAGGA | |
| | | CGCTATCAAGAAAGTGAAAAGATGATCCATAGTAAGGGAGAA | |
| | | AATATTTACAGATATGTATCAGATAAGGGATTGTAACCAGAAT | |
| | | ATATAAAGAAGTCTTACAACTCAATAATAAAAAATAAATAACC | |
| | | CAATTTTTTAAATGGGCAAAGGATTTGAATAGAAATTTCTCAA | |
| | | AAAATAATACAAATGGTCAATAAGAGCATGAAAAGATGCTCA | |
| | | GCATAGGTAGTCATTAGGAAAACGCATATCAAAACTACAAAGA | |
| | | GGCCGGGCATGGTGGCTCATGCCTGCAATCCCAGCACTTTGGG | |
| | | AGGCCGAGGCTGGCAGATCACCTTAGGTTAGGAGTTTGAGACC | |
| | | AGCTTGGCCAACATGGTGAAACCCCGTTTCTACTAAAAATACA | |
| | | AAAATTAGCCAGGCATGGTGGCACGTGCCTGTAGTCCCAGCTA | |
| | | CTTGGGAGGCTGAGGCAGGAGAATCACTTGAACCCAGGAGGT | |
| | | GGAGACTGCAGTAAGCCAAGATCGTGCCACTGCACTCCAGCCT | |
| | | GTGCAACAGAGTAAGACTCCATCTCCAAAAAAAAAAAAGAAA | |
| | | AAAGAAAAAAAACACAATGAGATACCACTTTACAGCCACTAG | |
| | | GATGGCTAGAATCAAAAGAATGGACAATAGTAAGTTTTGTTGA | |
| | | CAATGTGGAACCCTCACGCATTGTTGATGGGAACAAAATGGTG | |
| | | TAGTCACTTTGGAAAACAGTTTGATAGTTCGTCAAAATGTTAA | |
| | | ATATATAATTACCATATGACTCAACAATTTCACTTCTAGGTATA | |
| | | TACCCAAGAGAAATAAAGCATATGTCCACACAAACACTTGCAC | |
| | | ATAAATATTTATAGCAGCATCATTCATAATAGCTAAAAGGGAA | |
| | | AACAGCCCAAACATCCAACTGATGACAGACAAACAAAATGTG | |
| | | GCATATCCATACAATGGAATGTTATTCAGCCATAAAAAGGAAT | |
| | | GGGGAATGGATCCATACCACCACATAGGTAACCTTGAAAACAC | |
| | | TAACTGAAAGAAGCCAGATGTGAAGGTCACATATTGATACTTC | |
| | | TATTTATGTGAAATGTTCAGAATAGGAAAATCTATAGAGCCAG | |
| | | AAAGTAGATTTGTGGTTGCCAGGGGCTTTCGATGACAGGAGGG | |
| | | GAGGGGAGCAGTCATTGCTAGTGATTACAGGGTTCTTGAGAGG | |
| | | GAGTGAGAAAAAATGTTCTAAAATGTATTGTGGTGATGGTTGC | |
| | | ATAACTCTGTGAACATACAAAAACCATTGAATTCTATACTTTAA | |
| | | AAAGGTAAATTTTATGGGATGTGGATTATAGCTCAATAAAGCT | |
| | | GTCATTAAGAAAGGAAAAGGAAGGGAGGGGAGAAGAGGGAA | |
| | | AGGGGAGGGAGGGGAGGGGAGGGGAAGGGGGGAGAAAAAAG | |
| | | ATACATCGTGGCATATCTCTTCCCTTTTACCTGTCATTATGTGTC | |
| | | AGTGCCTCCCATTGGCCTAAACTACCCAGAAGCCAGAGGGAAA | |
| | | GCCATTTGGGACTATATTCCACTGTGACACTGAGCAGAGCAAG | |
| | | GGAAGGATGGGGACACTTCTCTCACACATCTCTGACTCCCCAA | |
| | | CATCTAACCCATGGCCTGGCATGTGATATTCATGCTATAAATAT | |
| | | TTGTTGAATCAGTAAATGGCCCCTCATCTAGATTCTGCCAGGCA | |
| | | GCGCAGGGGGCTTTTTGAATGTAAATGACCTAATGATTCAGGA | |
| | | TCCTATGACCAGCGACTGTCACCCTCTGTCACCTTTCTTCTGCC | |
| | | CCTTTATCTCTGAAGGCATTGAAGGGGCCCTGCATGAGTCAGG | |
| | | GAGGGCTGGGGGGAGGAGCAAGGGTGGGAGGGGCAGGGAAG | |
| | | AGGGTGGCTTCTCCGACAACTTAAAAGGGGCTTGAACCACTTC | |
| | | CTCCCCAGACAGGGGTAGTGCGAGGCCGGGCACAGCCTTCCTG | |
| | | TGTGGTTTTACCGCCCAGAGAGCGTC | |
| SB05120 | CXCL11 | TTAAAGAATTTAAATGCAGGATTATATCTAAATAAATATATAA | 134 |
| | | TTTCAAGATAAAATGCAAATGATAATTCTCAAAAAAAGAAGTT | |
| | | GAATAAGATAAAATAATAATCTAAAAGTAATAAACTGGCTATA | |
| | | AATTCCCCGATTATACCAACTTTTATTGGTAAAACTAGGAAATA | |
| | | GGAGAAAATTCTTGAAAGAAAGTTACAGTGATTTTCTCATGTT | |
| | | ACAAAGACAGGAGCTAAAAAAATATTATTGATGGTTGATGTAA | |
| | | ATAATTAGAAAAATTCAGGTAAAAATCTTTTTAAAGAATTTTA | |
| | | AGAATTAATATCAAAATGTAAAACAATGTATTCCTTTCAAATT | |
| | | ACTTAAGGAAGGCCAAGCACCGTGGCTCATCCCAGTAATCCTA | |
| | | GCACTTTGGGAGACCAAGGCAGGAGGATTGCTTGAGGCCTGGA | |
| | | GTTCATGGCTGCAGTGAGCTATGATTGTGCCACTCGACTCCAG | |
| | | AATGGTGACAGAGTGAGACCCATCTCATTAAAAAAAACAAAA | |
| | | ATGGCTTCAAAAAGTTAAATAAAATATATTTAAAGTTAGAAAG | |
| | | AAATCAAAGAAAACATAAAGAATCAGCAAGGAAGCATTTTAA | |
| | | AAATTACAGATTACCAGTTATACAGTAAATATATTTTATTAAGC | |
| | | TTACCCATTAGAAAGCTCCTTTGATATGATTTTCTTAAACATCT | |
| | | ACAAGTGACACACTTAAAACAAAGATTTAGGAAGCTTAAGAG | |
| | | GAAAAGATAAAGACAAAAAAATAGGAATTACATCAAAGTAGA | |
| | | ATCTCAGAGGTAAAACTATCACATGAGCAAAGAGGATCACTTT | |
| | | TTTTTATGATCCCTAATACAACTCATACAACTTCATGTACTAAG | |
| | | TAACAGTGCTAAAATATATATAAAGTAGCTTTCCACCATGGCC | |
| | | ACCATTGGAGGGCAGCAGCCATGACACTGCACTACCCTATAGC | |
| | | TGTGGGCCTCAACAAGGGCCACAAGGTGACCAAGAATGTGAG | |
| | | CAAGCCCAGGCACAGCCACTGTGGGCGCCTGACCAAACTCACT | |
| | | AAATTTGTGTGGGACATGATCCCAGAGGTGTGTGGCTTCACCC | |
| | | CTAACGAGTGGCGTGGCATGGAGTTACTGAAGGTCTCCAAGGT | |
| | | CAAACAGGCCCTCAAATTCATCAAGAAAAGGGTAGGGACAAA | |
| | | CATCTGCACCAAGAGAAGGCAGGAGGAGCTGAGCAACGTCCT | |
| | | GGCTGCCATGAGGAAAGCAGCTGCCAAGAAGGACTGAGCCCC | |
| | | CTGCCATCTGCCTATAATGAAAGCTTTGCAGAATAAAATAAAT | |
| | | ATAAAATAAAGTAATAAAATTAAATTAAATTTAAAAATAAAAT | |
| | | AAAGCAAAACAAAATAAAATATATAAAGTAAAAATTGTTGAA | |
| | | AATGCAAAACAATATGGACATAAATACAGAAACACAGGGAAA | |
| | | CTTCTTTAGGCACTCATTTACAGGTAAAAATATGAAATTGAATA | |
| | | AAGGTCATCTGGTGTCAAATAATATAGGCCTTATCTATTATAAG | |
| | | AGTTTGGACTGAAAAGCAAAAGTGAGATAATAAAAAAAAGCT | |
| | | TTTCAGAATATTATTTTGTATAGATATGTGAAGGATGAAGGGT | |
| | | GGGTGAAAGGACCAAAAACAGAAACACAGTCTTCCTGAATGA | |
| | | ATGACAATCAGAATTCCGCTGCCCAAAGTAGTCCGACAATTAA | |
| | | ATGGATTTCTAGGAAAAGCTACCTTAAGAAGGCTGGTTACCAT | |
| | | CTGGGTTTTCACAGTGCTTTCACATTCTTATCACTTTCAACACT | |
| | | ACTGCAAATAGGAAGGGACAGTAACATTTAGAAGAGAACAAA | |
| | | ACAGAAACTCTTGGAAGCAGGAAAGGTGCATGACTCAAAGAG | |
| | | GGAAATTCCTGTGCCATAAAAGGATTGCTGGTGTATAAAATGC | |
| | | TCTATATATGCCAATTATCAATTTCCTTTCATGTTCAGCATTTCT | |
| | | ACTCCTTCCAAGAAGAGCAGCAAAGCTGAAGTAGCAGCAGCA | |
| | | GCACCAGCAGCAACAGCAAAAAACAAAC | |
| SB05123 | GBP5 | ATGGCTGAAAACAGATGTTTTCTTTCAGAAATCCTTCCACTACC | 135 |
| | | ACTTCTGAGCATTTTCTCTTAGTATTTTTTTTTGGGCATTTTCCT | |
| | | TCTTTGCACTTTTTAAAATCTCCTGCAGCTCGCTCACTCTCTTTC | |
| | | ATAAGCCAGCAGTTATACTGATGTTAATAAATAGTTGTTTATTT | |
| | | ACTTTTGCTTTTTTAGAAAACAAGCTGAAAGAATTGAGTGAAA | |
| | | TGCCAGAGAGATACAGATAGCCAGTTGTAGGGCTGGGGTTGGA | |
| | | AATCAAGTCTAATTTAAGGCAAGGGCAAAATGTGCATTTTGTG | |
| | | TGTGTATTTTGAACCATTCTTGCATTGTTTTTATTTGCAAAGGTT | |
| | | ATAGGGCTCAAGCAGTTAGGGAGAATGCATCCCAGAACACTAA | |
| | | GTTATGAATAACAGAGCAACAGTTATGTTTGTGGCTTGAACAC | |
| | | ATTAAATCTGCAATAATGTTTTAGAAATTTGGCTTTACTAAAAG | |
| | | CATTAAAGATGCTATCCAGATGACATTGGGGACAACATGTCAA | |
| | | ATACTATTATTAATGTAGATATACAATGCTATTGTCATTCATAA | |
| | | GAGAGTCTGAACACTCAGACACATTCATTCCCTCTTCCTTAGTG | |
| | | ACATCATCCCAAATCATAAGATCTTGTAAGCTCTCAATATCTGC | |
| | | TTATAGATTTGGCTTATGTATCTTTTGTTTTTCTAGCTAATTGTT | |
| | | GTAGATCATCACTTCAAGGTGCCCATATCTTTCTAGTGGAAAA | |
| | | ATTATTCTGGCCTCCGCTGCATACAAATCAGGCAACCAGAATT | |
| | | CTACATATATAAGGTACTAATACTACATTGAAAATAGATTAGA | |
| | | ATGAGAAGAGACCTGCCATTTGAGTCTCTAAATTTGTTGCAAA | |
| | | ATTGCCTCTTTCTGGGTCAGGCTTGCACTAGGCATTTCCTGACT | |
| | | CCCATGTCCTAGTGCTACACCATTTGCAAATGGATATCCTATTT | |
| | | TCTGCTGCCAAGCAGTTTCTGTTTTAATTTATGTCAACATAATG | |
| | | ATTTCCAGGGCATTAGCTATGAGGAAGCAGGGAGAATGGGTCT | |
| | | TCATAATTGTAAAATGACAGTTTTTGAAATTTGCTAGTAAAGA | |
| | | GATGGAGAATCCAAACCTTTTCATTGTAAGATTTTAGTGAAAG | |
| | | AACTCGTCAAGCGGATAGTGAAGTTTTAGCATCCTTAGTAAAT | |
| | | AACCACCTTTAAAATGGAAGAAAACTATCCTTTATGATAAGGC | |
| | | AAAGTAGAAAAGCATATTTTTATGGCATTTGCTTTTTCTCATAT | |
| | | AGCTCAAACATTTTTTTTAACCAGAGACTCTTTAGGTTATAGGG | |
| | | ATTGATGCTCAGAAGTTAGAAAAAAATAGGTTAAGTATTATTA | |
| | | AAGGAAGTTGTTTTACTCATTCAGTCAATCATAAAATAATCATG | |
| | | CTTTGAAGTTTTGGTTTAATAAAGCACATTAGTAACTATGTAAA | |
| | | AAATTTCAAGTATATCACTAAGAAGCAAACAGCTATGATTTGT | |
| | | TAGATAATGCTGCATAATGACTTGGTAGTATTTAATGTAATGCA | |
| | | GTTTGACTGGACATAGATCTAACTGAAAAAAATGAAGTTGACA | |
| | | CAAAGTAAAAGGAACAGTGTAATATATTAATAAAGAACATCAT | |
| | | AAAGAATTAATATAGGAACCCAGGGAGGTAAAAACTAAAAAA | |
| | | CATAACTAAGCCAAAAACCAATGGAATAAGAGTTAGATTCAAA | |
| | | CTAGTAGGTTAGACGCAGGAAATGGATTTTCGTATGCCTATCA | |
| | | AATATTTAAGAACCAGTAGAATGGCCTATAGTAGACATATAAA | |
| | | GGGCCTAGGACTTACTTTTGTTTTAAAATAAAGTCTCTCTATTT | |
| | | TACAGGACTCTTTTTATTTTAAAACTTTTTTTTTCCAGAAACTTA | |
| | | GGCAGTGCTGGGGAGTAGTATGTCCCCAGGTTCAGTAAGCAAA | |
| | | ATGACTTCCTTTGCTTATTAAACAAACACCAGAAATTCCAGCCT | |
| | | ATCCCATCTGTTCCCTTGCAGGCAAAGTAACATCCTAGAC | |
| SB05125 | IDO1 | GCTCTTCTAAAAATATGCGAAATGAGGTTTTTAGGGAGGTGTA | 136 |
| SB09385 | | GGTATGGCTGAAGAAAATCAAGGTGAATGAAGACAAGATCAA | |
| | | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB05132 | UBD_1 | TCTCCTTCACCTTCCACCATGAGTAAAAGCTTCTTGAGGCCTCA | 137 |
| SB09406 | | CCAGAAGCAGATGCTGGTGCCATACTTCTTGTATAGTTGGAGG | |
| | | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB7017 | UNQ6494.1 | CAAGTCCACACTTTCTCTCCTCTCTCAGTGACAATCCAGTCCCA | 138 |
| | | GTAGGACGGTGACCCTGGGACTCGAGCTGTGTTCAGAGTCACC | |
| | | TCTTTTCGCTTTGATGTGCTCACTTTGCCTGATTTGAATTCACTC | |
| | | AGTCTTCCTCTTTCTCTGGCACATTCAATGCTTTGGGTTCAGTTC | |
| | | GTCTTGCTACCCAATTCCCTCCAGGGCTGACCATTTCCAAACCT | |
| | | TCAACCCTCACTCCCTTTCTCCCAATCCCACTCCAGCCTCACCT | |
| | | ACTCCACAGACCTTTCCAGGGGACGTTCAGCTGCTGCCCTTCTC | |
| | | TGTCCTCCCATGGACATACATTACCTTCTTCAGTTGCAGCTGAG | |
| | | GTGTCTTCACATTGCACTGTTTTCTCTAGTGTGTTTGTTCACGTG | |
| | | TCTTATTTTTGGCTTTCTTTATTAGCTGGAAGGGCCCTGGTGCG | |
| | | CCCACATCCCTCTCCAGAGAGTTCTGTCTGGTGATTTGCACACG | |
| | | TGAGGCCAGGAGAATTGCCATGTAAGGTCTGAGTGTCCACCTC | |
| | | TAGCTCTTTCTTGTCTCTTAAAGAAACAAAATACAGGGGCCGG | |
| | | GCACAATGGCTCATGCCTGTAATCCCAGCACTTTGGGAGGCCG | |
| | | AGGCCGGAGGATAGCTTGAGCTCAGGCATTCGAGACCAGCCTG | |
| | | GGCAACATGGTGAAACCTCATCTCTACTAAAAACACAAAAACT | |
| | | AACCAGGTGTGGTAGCACGTGCCTGTAATCCCAGCTACTTGGG | |
| | | AGGCTGGGACACGAGAATCACTTGCACCTGGGAGGTGGAGGTC | |
| | | GCAGTGAGCCGAAATCATAGCACTGCACTTCAGCCTGGGTGAC | |
| | | AGAACAAGACCCTGTCTCAAAAATATAAAATTAAATTTAAAAA | |
| | | TAATAAAATAAAATAAAATAAAATAAACAAAATACAGGGATG | |
| | | GGGGTGTGTGTTGATCCATGTGGCACCTCTTAGTGAGACTGCA | |
| | | ACAATCCCTTGATCCAGAAATAATCCCTGCTTCTTGCCCCTACA | |
| | | TCACAATTTACAATGTGGCACAATTATTCTGTAGCATTTGCATG | |
| | | TTAAATGGGCAAGAAAACTTAAAAAAGAATTCATTGAATGTCT | |
| | | CTGGTTAGAAAACAAAGACTCACCTCCTTTGTCAGAGAAGTGA | |
| | | CCTTTAGAAACCAGGCTTGCAGATGATACAAGATAAACTAAGA | |
| | | TAAACTTAACACCTGCAGTTTACACTGACATTCACATTTGCTAG | |
| | | CCTTAGGGACTCTCTCCAATGTTGTGAGGCTTTTCACATCACTT | |
| | | GAAAAGGAGCAAGAAAGACCTAGAATTCTTGAGTTTCATTAGG | |
| | | AACCTCCAGGGATGGAGGGGGCTGAGTATTTCTATCAGCCTGG | |
| | | TGGCTTGGAGCCCTGAGCTAGTGGCCCTCAAATATTTTAAGGTC | |
| | | GATTTGTGTCTGTCCTGAACCTCATTCACATGCAAATTCTTTTT | |
| | | AAATAGAAATTTGTAAATGGCGTGCACACCTCTAAGAGCGCTG | |
| | | CAGCTACTTGTCCAGTGTCTTGCCTTCTTTCTGAGTTACTGTTGG | |
| | | AGCCACGGCCAAAATTAGAGAGGCGTTTTCTGTAATGCTGAGA | |
| | | AAAGCAGGAAGCGGTGAGACGCTTGTAGCAGAACACGCAAAA | |
| | | CTGTTTGGCGGTGGCCCCCCCTCCACCTCCCACCCACCGCTCTG | |
| | | TAATTTCCCCAGGCAAGGCTGAAGGAGCCTTTCTTGGAAAACC | |
| | | CCTGATAACGTTGCTACACAAAGGTTGGGAGAGTCCCTGACGG | |
| | | CCTGTGAGGGACCATGTGGGGCTGGGGGTGGATAGGGGAGCTT | |
| | | GACTCTGCAGTCCCCAGGGCAGTCTGAGTCCCCAGTGCCTGGC | |
| | | GTGGGTGAGCTCAGGGCTGCTCCGTATGGGGGCATCCAGGCCT | |
| | | GGCTGCTGATGCCCTGTGGCTGAGGACACCTCCATCTGCATGA | |
| | | GCCAGTTTGGAAGTCCAGCTTGCCCCCCAGGCAGTGGAGGCCA | |
| | | GTAAGGGCTTCTCCCCAGATTGACCACTCAGATCAAATTGATC | |
| | | CA | |
| SB05140 | CD28 | GGTCATCAAAGAGTTTGACTGTAGCATTTTAAAATAGCGAAGA | 139 |
| | | AAAAAAGGTAACAACACTAGGGGTTTGTTAAACAATTTATGAT | |
| | | ATAATTAGAGAATGGAATATTATGCATCAATTACAAATAATGT | |
| | | TTATAAAGATACTTAAATGACATGGGAAAATGTTCAAGATATA | |
| | | ACAAATAATAGTACATATCTGATGAAAACTATAAGTAAAACAT | |
| | | GATATTAAACATGTAAAAATCTTAACAGACCAACAGGTGGAAG | |
| | | GCAGTGGACCAGAATATTAGCAGTGTTTATATTAGGGTGGTAA | |
| | | GAATGTGGATGAATCTTGTTTTAATGCTTATAATTTTTCTAAAC | |
| | | ATTCTCAATGAAATATGCCTTACTTTTATAGTCAGAATAAAAAG | |
| | | CAATACAAGTTATCTTTTAAAACAACCCACATACACAAAACCC | |
| | | CCAACTAGAGTAATTGCACAAAGCTTAGATTATCAAAAAGAAG | |
| | | AAAAAAATTACTGGAAAAGTTTTGAAGAAGTCATTGAGTACAA | |
| | | GATACTGACGTAGGTGCCGGAAGAAGAGAGCAGTTGGCCGTG | |
| | | CTGGTGGAATAACCCTCTCTGCAAAGGGCCTGGGAGTTGAAGA | |
| | | AGGGTGCAGTCGGGTGGTGGTTATGAATCTCAGAAATCCTGCC | |
| | | ACGGAGCCTCCTTTTGTGCCCTATTACTTAACCTTGAGGGACAT | |
| | | AGAGAGCATGAGACACCAAGGGGCTTTTGGTTGCCTTACTGTC | |
| | | CCACTAAGAACATAGAATGTTGTTTTGACTTTCCCTTTGCTTAG | |
| | | GGAACCTCCCCCAGATACTCAGCTGGCTGGCTGCTTGCACGTA | |
| | | GAATGGGTTTTGCAAAGTTCCTAGAAGTGAGTTGGAGGAGGCT | |
| | | TGACATAAATCAAGCACTGTGTGCTAAATGCTCCAGAGGGCTA | |
| | | CCTTATGTCCTACACAAATGTTACATTTCTAATATTTGTAACTC | |
| | | CTTTAAACGTTTATGCAGATGTTTCCCAATTCCTCAGTAGAATT | |
| | | CTTACCATTATCTTTTTGACACAACCTGTCCCCATCCTATGAAA | |
| | | ATCTCTCCTCTGCTAGAGACTTTATTCTTTCTTTCTCTCTCTCCC | |
| | | CCTTCCTTCCTTCCCTCCCTCCCTCTTTCTTTCTTTCCATCTTTCT | |
| | | TTCTTTCTTTCTTTCTTTCTTTCTTTCTTTCTTTCTTTCTTTCTTTC | |
| | | TTTCTCTTTCTTTCTTTCTTTTCTTTCTTTCTTTCTTTCTCTTTCTC | |
| | | TCTCTTTCTTTCTTCCTTTCTTTTTTCTCTCTCCCCTTCCTTCCTTC | |
| | | TTTCCTTCTTTTCTTTTCTTTTCTTTTCTTTTCTTTCTCTCTTTCTT | |
| | | TCTGTCTTTCTTTTCTCATTCTGTTGCCCTGGCTGGAGTGCAGTG | |
| | | GCATGATCTCGGCTCATAGCAGCCTCCACCTCCTGGGTTCAAGC | |
| | | GATTCTCCTGCCTTAGCCTCCCTAGTAGCTGGGATTACAGGTAC | |
| | | CCACCATGATGCCTGGCTAATTTTTTGTATTTTCAATGGAGACG | |
| | | GGGTTTCACCATGTTGGCCAGGCTCGTCTTGACCTCCTGGCCTC | |
| | | AAATGATCCACCCACTTTGGCCTCCCAAATTGCTGGCATTACAG | |
| | | GCGTGAGCCACTGCACCCGGCCTGTTCCTTCTTAAGAACACTTT | |
| | | GTCTCCCCTTTAATCTCTGCTGGATTTCAAGCACCCCTTTTACA | |
| | | CAACTCTTGATATCCATCAATAAAGAATAATTCCCATAAGCCC | |
| | | ATCATGTAGTGACCGACTATTTTTCAGTGACAAAAAAAAAGTC | |
| | | TTTAAAAATAGAAGTAAAAGTCTAAAGTCATCAAAACAACGTT | |
| | | ATATCCTGTGTGAAATGCTGCAGTCAGGATGCCTTGTGGTTTGA | |
| | | GTGCCTTGATCATGTGCCCTAAGGGGATGGTGGCGGTGGTGGT | |
| | | GGCCGTGGATGACGGAGACTCTCAGGCCTTGGCAGGTGCGTCT | |
| | | TTCAGTTCCCCTCACACTTCGGGTTCCTCGGGGAGGAGGGGCT | |
| | | GGAACCCTAGCCCATCGTCAGGACAAAG | |
| SB05149 | PLXDC1 | GCACTGCACTCCAGCCTAGGCAACGTGCGAGAACCTGTCTCAA | 140 |
| | | AAAACAAACAAAATACACACACACACACACACACACACACAC | |
| | | ACACACACACACACACACCCTGCAGGTGATTACGATCTCAAGT | |
| | | TCTAGAGGCTTACGTTAAAATTTACACATTTGCTTCTACTATTT | |
| | | ACAAGTTTATGTTTCAGCCTGAGCTCTTCTTGTAAGACAGACCA | |
| | | CGAGGTACCCCAGATGGAGGAATGTGCAAAGACGCCGGGGCT | |
| | | GGAGCTGGAATCCGTCCCGAGACTTCTCACCTTGACCTCGAGG | |
| | | AAGGCAGGAGGTCGGAGACAGGGGGTGGGCAGGCTGGGGAAA | |
| | | GAGGAAACCCGCAGGACAAGAGGGTTGTGGCTCGTCACGTGG | |
| | | CCACTTGAGGGCAGCAAGTAGCTGCAGCTGGAGGTAGCTGCCT | |
| | | ATCTGATTCTGTTTGAGAGTCCGCCAAGTTGGGATTTAAATTAG | |
| | | ACATCAAGAAGAATTTACAGACCGGCCTTGCCTTGTTCCACGG | |
| | | TAAGGGGACATTGGGTTTAGGGCTGGAGGAGGTAGTCTCTGGG | |
| | | ACTCTGGGCCCGGCGGAAGGAGGAACCGGCGTTCTAAGAGATT | |
| | | AGAGAGCCTTTAGTTGTTTCCTTTCTTAGGTGTTTTTTTTTCTTT | |
| | | TCTTTTCTTTTTTTTTTTTTTAACCTCAGTGCCACGGAATCCGTC | |
| | | TCTTCCAGCCCACAGTGTACCTTCTCCTGTCCCCGCCCATATCC | |
| | | CTCCTCCTGGAAAATTTCTCCAATGGCGCTCAGCGTTCGCACCC | |
| | | TTCCTCTCCGCGGTTTTCTAGTCCCTAGGCCCCAGCCCACTAGA | |
| | | AGCCAGGATGCCCCCTCACCCCGTCTCCCACTCTCCAGCGCCCC | |
| | | AGCTCCTTGCCGTCACAAGAACGAAGTAGGTCAGGGGAGGGA | |
| | | AGAGGCATCTTCGTGTCCTGACCTGATTCCTTGCCGTCTATTAA | |
| | | ATATCCTACATTGCGGGTATTTTTATTCTGTCCTGGAAGAAAAA | |
| | | ATAAAAAGATTATCTATCCCCAGAGCCTGTCAGAAGGTGGTGA | |
| | | CAGGTGGGGTGAGCCATTTCCTCGTCTGGGGCTGGCTCTGAAG | |
| | | ATTGATTGGGTCTGGCCCTGAGTAGGCCTGGAGGCCCCAGAGA | |
| | | GGCAGGAGGCCTGGGTTACCTACTCGTGGGGAGGGGTCCAGAC | |
| | | TGTTGCAGAGTAAGGGGTAGCCAGGCCCCCTCCACCCTCACGT | |
| | | CTGCTAGGCTTGGGGGGCAAATCGGTGCTGGAAATGGGGGGTG | |
| | | GAGGGATAAGGTGGAGTCATGGAAGGGGGGTGGGATTCAGGC | |
| | | CCCTCTTGGGGAGAAAAGCTGGGGGTCTTTGCTGCAGGGGGAG | |
| | | ATTTCTGGGGGTGGGGGCTGGAGGACAAAGCATCAGAGACGC | |
| | | ACAGAATTCCGAAGAACAGCTCAGGAAGTCGCAGAGCTTGGA | |
| | | GGAGCTGGGGGCGGGGAACAGTCAGGGGCCCGTGACCCAGGG | |
| | | ATGGGGCGCCTGTCCCTTCCAGGGGTCCAGTCCTCGGGCCTTCT | |
| | | CTCTGCAGCTTTCCCGGCGGAACGGAGGAGGGAAAGGGAGCC | |
| | | AGGAGCTCAGTGAGGAGCGCGGGGCGGCTGATGCTTCTAGGG | |
| | | GTTCTCTCAGCCCGAGGGGCTCCCGCCCTGTCCCCTACTCTGCA | |
| | | CCCCTTGCCTTGGGAGCGGCTGCAGAGCTCTAGGGGAGCGCTC | |
| | | CGTTAGGGCTGAGGGCAGGCGCCGGGAGAGCCCCCAACGACG | |
| | | CCGCGCCCCCCGCCCCGCCCCCTGCCCCAGCTCCCCCCCACCCC | |
| | | CACCGACCCAGCGGCCCCCCCAGCTCCCCGCCTGCTCCCGGAG | |
| | | GCCGCAGCCTCCAGCTCCGCTCGCGCTCTCGCCGCTCCTGCCGG | |
| | | CTCGCCCGGCCCCGCGCTCCGCCGTCTCCTCGCCGCCCGCCCCT | |
| | | CCGCCAGCCCCGGGGACCGCGCGGCCGCAGCCTGAGCCAGGG | |
| | | CCCCCTCCCTCGTCAGGACCGGGGCAGCAAGCAGGCCGGGGGC | |
| | | AGGTCCGGGCACCCACC | |
| SB05152 | ZNF704 | ACACTCCGTGCTGGGAGCACCACTACTCTCTTCAAAGCTGTCA | 141 |
| | | GACAGGGACGTGTAAGTCTGCAGAAGTTGCTGCTGCCTTTTGTT | |
| | | CAGCTGTGCCCTACCCCCAGAGGTGGAGTCTACAGAGGCAGGC | |
| | | AGGCTTCCTTGAGCTGCAGTGGGCTCCACCAGGTTCGAGCTTCC | |
| | | CAGCCGTTGTGCTTACCTACTCAAGCCTCAGCAGTGGCTGATGC | |
| | | CCCTCCCCCAGCCTTGCTGCCGCCTTGCAGTTTGATCTCAGACT | |
| | | GCTGTCCTTGCAGTGAGCAAGACTTCGTGGGTGTGGGACCCTC | |
| | | CGAGCCAGGCACGGGATATAGTCTCCTGGTGTGCTGTTTGCTA | |
| | | AGGCCATTGGAAAAGCGCAGTGTTAGGGTGAGAGTGTCCTGAT | |
| | | TTTCCAGGTACCATCTGTCACGGCTTCCCTTGGCTAGGAAAGGG | |
| | | AATTCCCTGACCCTTTGCACTTCCCGGATGAGGCGATGCCCCAC | |
| | | CCTGCTCTGTGGGCTGCACTCACTGTGTGACAAGCCCCATTGAG | |
| | | ATGAACCTGGTACCTCAGTTGGCAATGCAGAAATCCCCTGTCTT | |
| | | CTGCATCACTCAGGCTGGGAGCTGTAGACTGGAGCTGTTCCTA | |
| | | TATTTGGCCATTTTGGAACTTCCATCCTGACAATGGGCTAATAT | |
| | | CCAGAATCTACAAAGATCTTAAACAAATTTACAAGAAAACATC | |
| | | AAACAACCCCATCAAAAAGTAGGCAAAGGATATGAACAGACA | |
| | | CTTCTCAAAAGAAGACATTTGTGCAGCCAACAGACACATGAAA | |
| | | AAATGCTCATCATCACTGGTCATCAGAGAAATGCAAATCGAAA | |
| | | CCACAATGAGATACCATCTCACACCAGTTAGAATGGCAATCAT | |
| | | TAAAAAGTCAGGAAACAACAGGTGCTGGAGAGGATGTGGAGA | |
| | | AATAGGAACACTTTTACACTGTTGGTGGGACTGTAAACTAGTT | |
| | | CAACCATTGTGGAAGTCAGTGTGGCGATTCCTCAGGGATCTAG | |
| | | AACTAGAAATACCATATGACCCAGCCATCCCATTACTGGGTAT | |
| | | ATACCCAAAGGATTATAAATCATGCTGCTATAAAGACACATGC | |
| | | ACACATATGTTTATTTTGGCATGATTCACAATAGCAAAGAGTTG | |
| | | GAACCAACCCAAATGTCCCATCAATGATAGACTGGATTAAGAA | |
| | | ACTGTGGCACATATACACCATGGAATACTATGCAGCCATAAAA | |
| | | AAGGATGAGTTCATCACATGTTCTCACTCATAGGTGCGAATTA | |
| | | AACAATGAGAACACTTGGACACAGGGTGGGGAACATCATACA | |
| | | CCGGGGCCTGTCATGGGTTGGGGGGGGGGGGGAAGGGATAGC | |
| | | ATTAGGAGATATACCTAATGTAAATGATGAGTTAACGGGTGCA | |
| | | GCACACCAACATGGCACATGTATACATATGTAACAAACCTGCA | |
| | | CGTTGTGCACATTTACCCTAGAACTTAAAGTATAATTAAAAAA | |
| | | TTAAAATAAATTAATAAAAAAATCTTAACTGTTGGGGATGAAG | |
| | | GATAAGAAATAATTCTTCATGAACTACAGAGTGTGGTTAAGAC | |
| | | CAGTATTATTAGCCAAAAAGAAAGTTAGATTTTATACATTAGC | |
| | | TCTCTTCTATTAAACATGATAATCAGACTGAAATATTGAAGAGT | |
| | | TTACTTGAATTTTCTTAGTTGTGGGGGATAAAACAATGATGTTT | |
| | | TACATTTTAAAAAATTAGAGGTATTACACTTTTCAAATCTTTGT | |
| | | TTTTGTATACCTGGGAGGCATTTTTTTGTGTGTTTGTGATGTTTC | |
| | | TTATTTGCATTATTACAGATGCATTGATTGTATATTATTTTGTGA | |
| | | AGTTATTTTATTTCTATGTTGCATTTTTCTTATTAAAATTATACA | |
| | | ATCTTTCAGATGAACAGGAAGGACAGTGTCTGTAATCTCTCCTC | |
| | | ACCGCCATCTGCAGTAGTCGTACAAAGGTGATGTTTTATGTGA | |
| | | GTAAGAATTTCTGTGTTTCATTTCACAGGTGGGGAGCATTAAGC | |
| | | GGGAA | |
| SB09119 | IL7R | GCCCAGAGAAGGCCCTGACAAATGGAGAAGATGGAATATGTC | 392 |
| | | CCCTTCAGTTGGATTCTTGCTCCGAAGTATTCCTTGAGCCTATA | |
| | | CTCCAAAGTGTGGCATTATAACTGGTGCAGACGGAGTCTGTCT | |
| | | TAAAACTGAAAAGGAGGATCCAGAGACTGGTACAAATGTCCC | |
| | | ACCTCCTTTTTTTGTTACTTCGTTATTGTCAAATAATTGTAATGA | |
| | | TGGGACAAGCTAAATCATGAGTCTTTTAAAGTCTCTTCGATTTG | |
| | | CTTTACACCTGGGGGGTTGTTGCTTGTCTCTGCTAATTTTAGCC | |
| | | ATTTCACAGGCCCATGGGAGCAATTCTACCTAATGACAGTGAA | |
| | | TCCAACAGAAGTTCATAACTCACTGATCACTTAGTTTCTCTGCA | |
| | | CTCCTCACCTTATAACTGCAGTCTTGTGGCATTTTTCCAAAAGA | |
| | | GAGCCACTGTGAATCAAGCATAGAGTTTTATGGCCTGAATCCA | |
| | | GGTTAGATTATTTTTTTCAAGAAAAAGAAAAAAAAATCTAACA | |
| | | GTTACCAAGAAGCAGTCAGGCTTTACAAATATCACCACTTTAA | |
| | | CCATGAGGCCATATTAGGAAGGCTTTTGACTGGCATTCAGGTTT | |
| | | GGGGGAGTCTGAATTTTCTATGCCATGGTCTCAGATTTCTGCCC | |
| | | CATATACCTAGGCACTAATTTAGTTCATATGTACTATGTGTACC | |
| | | TGAAAAGTTGTGTGGCAATCAAATTTTCACAAATAGAATCCTG | |
| | | TTTTAAATACACTAAGAAAGTACCTACTTTATCCTTTAAACAAG | |
| | | AGGTCAGCAGACTTTTTCTACAAAGGGTCAGATAGTAAAGATT | |
| | | TTACACCTTTTGTACAATACAATCTCTATCTCATCTACTTAGCTC | |
| | | TGCCATTGTTGCATAAAAGCAGCTGTAGATGATACACAAATGG | |
| | | GTGAGGCTGTATTCCAATGAAACGTTATTTGCAAAAACAGGTG | |
| | | GTAGATTAAATTTGGTCCCAAGGCTTACTTGGGAAAAAAAAAG | |
| | | ATCTTTTGAAAAAGAAAAAATAAATGAATAATTTTTTAAAAAA | |
| | | TTGTTCCCTAGGTCATAGTTTGCCAGCCCCTGCCCTAAACAAAT | |
| | | AATTCTTGAATGCCTACTGTGGTGTGTAAGATATGAGTAAATA | |
| | | CCAGGGATACACAGAGAACAAAAGAGAAAAACTGCTATTCTT | |
| | | GTGAAACTTGGAAGTTGGAGGTAAGCTATTTAAAATAAACCCA | |
| | | CAATAAAGTACTTCACATAGTGCAGACTGTTTCTTTAAATCAAA | |
| | | ACTCACTCCAAACAACCAATTGATTCACTTTGTAAGTTTGAATT | |
| | | TTTGTCTTCAGATTCTTTTAAAGTGGGCCCTTAGTCAGGAGCGG | |
| | | TGGCTCATGCCTGTAGTCCTAGCACTTTGGGAGGCTGAGGCAG | |
| | | GCAGATCACTTGAGGTCAGGAGTTCGAGACAAGCCTGGCCAAC | |
| | | ATGGCGAAACCCCGTCTCCACTGAAAACACAAAAATTAGGCTG | |
| | | GCATAGTGGCATTTGCCTGTAGTCCTAGCTACTCAGGAGGCTG | |
| | | AGGCAGGAGAATTGCTTGAACCTGGGAGGTGGAAATTGCAGTG | |
| | | AGCCGAGATCATGCTATTGTACTCCAGCCTGGGCAACAAAGCA | |
| | | AGACTCTGTCTCAAAAAAATAAAAATTAAAAAAATAAAGTAGC | |
| | | CTCTAGCCTAAGATAGCTTGAGCCTAGGTGTGAATCTACTGCCT | |
| | | TACTCTGATGTAAGCACAGTAAGTGTGGGGGCTGCAGGGAATA | |
| | | TCCAGGAGGAACAATAATTTCAGAGGCTCTGTCTCTTCATGTCC | |
| | | TTGACCTCTGCTTACAGCAGCAATACTTTTACTCAGACTTCCTG | |
| | | TTTCTGGAACTTGCCTTCTTTTTTGCTGTGTTTATACTTCCCTTG | |
| | | TCTGTGGTTAGATAAGTATAAAGCCCTAGATCTAAGCTTCTCTG | |
| | | TCTTCCTCCCTCCCTCCCTTCCTCTTACTCTCATTCATTTCATAC | |
| | | ACACTGGCTCACACATCTACTCTCTCTCTCTATCTCTCTCAGA | |
| SB06376 | SOCS3 | AAGCGATCCTTCCACCTTGGCCTCCCAAGCAGCTGGGACTACA | 393 |
| | | GGCACAGGCCACCACTCCTGGCTAACTTTTTTCTATTTTTTGCA | |
| | | GAGATGGGGGTGCTCACTTTGCTGCCCAGGCTGGTCTTGAATG | |
| | | TCCATGCTCAAGTGATCCTCTCGCCTCCGCCTGCCAAAGGGAA | |
| | | GGAAAAGGTTCTGGCTCCAGTTTTACAGGTGGCAAAACGGAAG | |
| | | CCCAGAGAAACTGTGTGTCTTGTCCAAATCTCACTGAGTCCCCA | |
| | | GCACATGCTGGGGAGAGGTGATGGCACGGACTGAGGATACAG | |
| | | TGGCAAAGGCAGTGCGCACCTATCAATTCCAGGGCCGGCAAAA | |
| | | GGGCCAGTTCTGGGTTCTGGCATCCCAAGCTCCCTGTCCCTGGC | |
| | | GGCAGTCTCTTCCTTCCCTTTCAGCACCTCATTATCCTGGTATCT | |
| | | AGGAAGAGCGCAGGGCCCCAGGCCCACAGCGTCTGCATCCCG | |
| | | AGAGGTGAAAATCTAAATGCCACTGAGGCAGCTCTCGGGTGTC | |
| | | CCCAGCACCACACAGCCCATTTGGGGACAAGATGACAAAACTT | |
| | | GCCATCCTTCATAAGGCTGGCCCCGGGTCTCCAAGCCCAAACT | |
| | | ATCTACGTCAGTGCCAACGTGACCAGCAGCAAACCAATCTTTC | |
| | | ACCTCAATCACTTCCTCTCCTCCCCTCTACCCCCTCCCCGCCAG | |
| | | TGATCAAGTCCTCCCCCACCCCAAGAGAGGCAGGGGGTCACAT | |
| | | TCCAGGGGGTGTGGGCCATCTGGCAGTAGCCTGAACCCCGGAA | |
| | | AACTGCTTTCCGGAAATTCTCTCCTGCTAGAATTTTATTCCCAG | |
| | | GTCTGACCCCAGCCTGGGGTGTGAATGCCCCCTTCTCGGCCACC | |
| | | TTTCCAGCTCCGGAGACAGCCATTCCCGCAGATCCCTGGCGTG | |
| | | CCTATTCCAAGGCCTGGGCCGCGCAGCACCAAACTGCCCTCCC | |
| | | CTAGGAGACCCCAGGGCGCCCTCTGGCGGACGCGGGCCGTAGG | |
| | | TTGGGGCCATCGTTCCACCTCGAGCTCTCCAGCCCGGCCGAGA | |
| | | GCACCTGGCCCAACCCACACCGGACCAACCAGGTTTGCAGACC | |
| | | CCAGTGCATGAAAGCGTTTTCATAGGGGAGGGGAGGGGGGCTC | |
| | | CACACTCGCGTCAGGGTTGGCAAAGAACCTGGCAGTGGGCCGA | |
| | | GGCTGGGTAGCTGCCACGAAGGGGCGTCCCCTGCAGCCCTGGG | |
| | | TCTCCCCTCTGGAATCTGCCCGCAGGTGACTGTCGCACGTCTCC | |
| | | AACCTCCGGCTCCCGGGTCTGACCCCAGCCCCGCTGCCTGGCT | |
| | | GTGGGGTAGCCCCACTTCCCTTTCGGCTGGCCCGGGCCGCCTG | |
| | | ACCCGCAGTTGGGCCCTCGGGGAGGCCCGGGTGGCGGCGAGG | |
| | | GGGTGTCCCCGCGGGGTCCGGGAAAAGGGGAAGGGGAACCGG | |
| | | GAGGCTCTCCAGGTCGGCCTCCTAGAACTGCCCGCTCTCCCGA | |
| | | AGCGGCGCCCCCGCCTCTGCCAGAAATCAGCCTTCTTAGAAGG | |
| | | GAGGGGGGTGGGGGGAAAGTGTGAATGAGAAGTTGGGGGCGG | |
| | | AGCGCGCGGCGGGGAGGGGCCGCTGCCAGGAACGCGCCGCCG | |
| | | GGGCTGGCGCCGCGCCCACCGGCCGCCTCGGCCGCCTCTCGTC | |
| | | GCGCTTTGTCTCCGCGCGCGTCCCTCCCGGTCCCTGCCCCTGCT | |
| | | CGCGGCCCGCCCTCGGCGCCCGCGGCCCCTCCCTCACCCTCCGC | |
| | | GCTCAGCCTTTCTCTGCTGCGAGTAGTGACTAAACATTACAAG | |
| | | AAGGCCGGCCGCGCAGTTCCAGGAATCGGGGGGCGGGGCGCG | |
| | | GCGGCCGCCTATATACCCGCGAGCGCGGCCTCCGCGGCGGCTC | |
| | | CGACTTGGACTCCCTGCTCCGCTGCTGCCGCTTCGGCCCCGCAC | |
| | | GCAGCCAGCCGCCAGCCGCCCGCCCGGCCCAGCTCCCGCCGCG | |
| | | GCCCCTTGCCGCGGTCCCTCTCCTGGTCCCCTCCCGGTTGGTCC | |
| | | GGGGGTGCGCAGGGGG | |
| SB07097 | CCL19 | GATGAATTAATGAACAGATACCTGAATAAAAGGATAGAAGGC | 142 |
| | ablation | CAGGCACGGTGGCTCACGCCCTTACCTACTACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07098 | CCL19 | GATGAATTAATGAACAGATACCTGAATAAAAGGATAGAAGGC | 143 |
| | ablation | CAGGCACGGTGGCTCACGCCTGTAATCCCAACACTTTAATTCA | |
| | variant | GACGACAAACCATTCTATTTGAAGTCAGGAGTTCGAAACCAGG | |
| | | AGTTCGAAACCAGCCTAGCCAACAAGGTGAAACCTCACCTCTA | |
| | | CTAAAAATACCAAATTTAGCCGGGCATGATGGTGGGTGCCTGT | |
| | | AATCCCAGCTACTCAGGAGGCTGAGGCAGGAGAATCCCTTGAG | |
| | | CCCAGGAGGTGGAGGTTGCTCTGAGCCAAGATCGTGCCATTGT | |
| | | ACTCCAGCCTGGGTGACAGAGTGAGACTCTGTCTCAAAAAAAA | |
| | | AAAAAAAAAAAGGACAAAAGAAGAACAATGGCTAACAGAGCT | |
| | | TATTGTGCGTCAGCCTCTAAGCTAAGGGCTTTACTCAGGGTTCC | |
| | | TCACTTAATCCTAAGAAGTAGATCTTCTACCATTCCCATTTTAC | |
| | | AAATAAGGACACTAAGACACAGAAACTGGAGAATTTTCACCTT | |
| | | GTCGCAGTTAGTAAGTGGTGGAATCTGAATCCCAAGCTAGGCG | |
| | | CTCTGTCTGCAGAGTCTCCCTCCTCTGCACCGTGACTACCCGTG | |
| | | CTGTGCCATGACCAAGTGGCAGACAGGCGGTGTGGAGTGGAG | |
| | | GTAAAGACTGCAAACCCTAGAGCACACCTGGGTCCAAATCATG | |
| | | CTCCAGTACTTACTAGCTGTGTGGCTTTGTGCTCTGAGCCTCAT | |
| | | GTTCCTTATGTGTAAAATAAGAATAATAATGTGAAAATAAGAA | |
| | | AAACTACATCAATGGTATTGTTTTGGGAAAGTTAAGCAAGGTA | |
| | | ATAGTGGTAAAGTCCTTGACATAGAGCCTAGCACATAGTAAAC | |
| | | AATCAAAAATACCATTACTATTATTATGATTAATGAATGGAAA | |
| | | GAAGCTAAGTCAGAAATTTAAGAGGGAAATGTTCCCCCTCCCT | |
| | | CTCTCCATCCAAAAGAGGGTCTAGGGTTTGGGTGACGACGGGT | |
| | | GACTGGGCGTTTCACATTTAGTTTCTCTTTCAAGGCCTTCTCTTC | |
| | | TGACCTTTCTCATAAGGATGGGAAGTCACTTGTCCCCTCCCCCA | |
| | | TCGGCCGATTCCTTGAAATTTTAAGATTTGTCTGAATATGAGCT | |
| | | GGCCAGCCCCTCTCCTTAGAAATCCCCTCTTGGTTCCCAGGACT | |
| | | GAGAAAAGGGGGAAAATGAAGGGGGCTATTCCAAGCCAGTCT | |
| | | CCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07099 | CCL19 | GATGAATTAATGAACAGATACCTGAATAAAAGGATAGAAGGC | 144 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCATTCTAAGTCCAATTCACGACACTCACCTCTA | |
| | | CTAAAAATACCAAATTTAGCCGGGCATGATGGTGGGTGCCTGT | |
| | | AATCCCAGCTACTCAGGAGGCTGAGGCAGGAGAATCCCTTGAG | |
| | | CCCAGGAGGTGGAGGTTGCTCTGAGCCAAGATCGTGCCATTGT | |
| | | ACTCCAGCCTGGGTGACAGAGTGAGACTCTGTCTCAAAAAAAA | |
| | | AAAAAAAAAAAGGACAAAAGAAGAACAATGGCTAACAGAGCT | |
| | | TATTGTGCGTCAGCCTCTAAGCTAAGGGCTTTACTCAGGGTTCC | |
| | | TCACTTAATCCTAAGAAGTAGATCTTCTACCATTCCCATTTTAC | |
| | | AAATAAGGACACTAAGACACAGAAACTGGAGAATTTTCACCTT | |
| | | GTCGCAGTTAGTAAGTGGTGGAATCTGAATCCCAAGCTAGGCG | |
| | | CTCTGTCTGCAGAGTCTCCCTCCTCTGCACCGTGACTACCCGTG | |
| | | CTGTGCCATGACCAAGTGGCAGACAGGCGGTGTGGAGTGGAG | |
| | | GTAAAGACTGCAAACCCTAGAGCACACCTGGGTCCAAATCATG | |
| | | CTCCAGTACTTACTAGCTGTGTGGCTTTGTGCTCTGAGCCTCAT | |
| | | GTTCCTTATGTGTAAAATAAGAATAATAATGTGAAAATAAGAA | |
| | | AAACTACATCAATGGTATTGTTTTGGGAAAGTTAAGCAAGGTA | |
| | | ATAGTGGTAAAGTCCTTGACATAGAGCCTAGCACATAGTAAAC | |
| | | AATCAAAAATACCATTACTATTATTATGATTAATGAATGGAAA | |
| | | GAAGCTAAGTCAGAAATTTAAGAGGGAAATGTTCCCCCTCCCT | |
| | | CTCTCCATCCAAAAGAGGGTCTAGGGTTTGGGTGACGACGGGT | |
| | | GACTGGGCGTTTCACATTTAGTTTCTCTTTCAAGGCCTTCTCTTC | |
| | | TGACCTTTCTCATAAGGATGGGAAGTCACTTGTCCCCTCCCCCA | |
| | | TCGGCCGATTCCTTGAAATTTTAAGATTTGTCTGAATATGAGCT | |
| | | GGCCAGCCCCTCTCCTTAGAAATCCCCTCTTGGTTCCCAGGACT | |
| | | GAGAAAAGGGGGAAAATGAAGGGGGCTATTCCAAGCCAGTCT | |
| | | CCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07100 | CCL19 | GATGAATTAATGAACAGATACCTGAATAAAAGGATAGAAGGC | 145 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCGT | |
| | | TGAAGCTTGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07101 | CCL19 | GATGAATTAATGAACAGATACCTGAATAAAAGGATAGAAGGC | 146 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCGAGTCGTCAGACTCAATTATTACCTTGAG | |
| | | CCCAGGAGGTGGAGGTTGCTCTGAGCCAAGATCGTGCCATTGT | |
| | | ACTCCAGCCTGGGTGACAGAGTGAGACTCTGTCTCAAAAAAAA | |
| | | AAAAAAAAAAAGGACAAAAGAAGAACAATGGCTAACAGAGCT | |
| | | TATTGTGCGTCAGCCTCTAAGCTAAGGGCTTTACTCAGGGTTCC | |
| | | TCACTTAATCCTAAGAAGTAGATCTTCTACCATTCCCATTTTAC | |
| | | AAATAAGGACACTAAGACACAGAAACTGGAGAATTTTCACCTT | |
| | | GTCGCAGTTAGTAAGTGGTGGAATCTGAATCCCAAGCTAGGCG | |
| | | CTCTGTCTGCAGAGTCTCCCTCCTCTGCACCGTGACTACCCGTG | |
| | | CTGTGCCATGACCAAGTGGCAGACAGGCGGTGTGGAGTGGAG | |
| | | GTAAAGACTGCAAACCCTAGAGCACACCTGGGTCCAAATCATG | |
| | | CTCCAGTACTTACTAGCTGTGTGGCTTTGTGCTCTGAGCCTCAT | |
| | | GTTCCTTATGTGTAAAATAAGAATAATAATGTGAAAATAAGAA | |
| | | AAACTACATCAATGGTATTGTTTTGGGAAAGTTAAGCAAGGTA | |
| | | ATAGTGGTAAAGTCCTTGACATAGAGCCTAGCACATAGTAAAC | |
| | | AATCAAAAATACCATTACTATTATTATGATTAATGAATGGAAA | |
| | | GAAGCTAAGTCAGAAATTTAAGAGGGAAATGTTCCCCCTCCCT | |
| | | CTCTCCATCCAAAAGAGGGTCTAGGGTTTGGGTGACGACGGGT | |
| | | GACTGGGCGTTTCACATTTAGTTTCTCTTTCAAGGCCTTCTCTTC | |
| | | TGACCTTTCTCATAAGGATGGGAAGTCACTTGTCCCCTCCCCCA | |
| | | TCGGCCGATTCCTTGAAATTTTAAGATTTGTCTGAATATGAGCT | |
| | | GGCCAGCCCCTCTCCTTAGAAATCCCCTCTTGGTTCCCAGGACT | |
| | | GAGAAAAGGGGGAAAATGAAGGGGGCTATTCCAAGCCAGTCT | |
| | | CCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07102 | CCL19 | GATGAATTAATGAACAGATACCTGAATAAAAGGATAGAAGGC | 147 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAAATTGGAACCACGTATCTACTGCATTGTAACTACAAC | |
| | | AGCTCGAGGTATTAGATAAGAAGAACAATGGCTAACAGAGCTT | |
| | | ATTGTGCGTCAGCCTCTAAGCTAAGGGCTTTACTCAGGGTTCCT | |
| | | CACTTAATCCTAAGAAGTAGATCTTCTACCATTCCCATTTTACA | |
| | | AATAAGGACACTAAGACACAGAAACTGGAGAATTTTCACCTTG | |
| | | TCGCAGTTAGTAAGTGGTGGAATCTGAATCCCAAGCTAGGCGC | |
| | | TCTGTCTGCAGAGTCTCCCTCCTCTGCACCGTGACTACCCGTGC | |
| | | TGTGCCATGACCAAGTGGCAGACAGGCGGTGTGGAGTGGAGGT | |
| | | AAAGACTGCAAACCCTAGAGCACACCTGGGTCCAAATCATGCT | |
| | | CCAGTACTTACTAGCTGTGTGGCTTTGTGCTCTGAGCCTCATGT | |
| | | TCCTTATGTGTAAAATAAGAATAATAATGTGAAAATAAGAAAA | |
| | | ACTACATCAATGGTATTGTTTTGGGAAAGTTAAGCAAGGTAAT | |
| | | AGTGGTAAAGTCCTTGACATAGAGCCTAGCACATAGTAAACAA | |
| | | TCAAAAATACCATTACTATTATTATGATTAATGAATGGAAAGA | |
| | | AGCTAAGTCAGAAATTTAAGAGGGAAATGTTCCCCCTCCCTCT | |
| | | CTCCATCCAAAAGAGGGTCTAGGGTTTGGGTGACGACGGGTGA | |
| | | CTGGGCGTTTCACATTTAGTTTCTCTTTCAAGGCCTTCTCTTCTG | |
| | | ACCTTTCTCATAAGGATGGGAAGTCACTTGTCCCCTCCCCCATC | |
| | | GGCCGATTCCTTGAAATTTTAAGATTTGTCTGAATATGAGCTGG | |
| | | CCAGCCCCTCTCCTTAGAAATCCCCTCTTGGTTCCCAGGACTGA | |
| | | GAAAAGGGGGAAAATGAAGGGGGCTATTCCAAGCCAGTCTCC | |
| | | CTCCAGCTGTTCCTTTGACCCAGGTGTTGGACATAGGAAAACA | |
| | | GTAAATAGAGGAAGGGGAGGGTTTGCTGAGCCCCAGAGGCCT | |
| | | GAGATGTGGGATGGGGGTCCTGGGCCTCCTCCCAAGGTACCTG | |
| | | GCCTTTCAGCTCTGCTTGCCCCAAGGGAAGGACTGGTTCCCAC | |
| | | ATAGTGGGCAGGCACAGTGATGACCTTGGAGGCCACCCCAGGA | |
| | | TCTCATGGTTGTCCTGAGGGTGCCAGGGAGCCTTCTTCACCACC | |
| | | AGCCCAAGAAAGAAGCACCAGTGAGGACAAGGGATAAACCTA | |
| | | AGGAAGGGAGCACAGTTCCTCACTGTGGGCCCCACCTTTGACC | |
| | | ATCACTTCTGAACCCCAGCTTCATGGTTGATGATGCAAATGCCA | |
| | | TCAATGGGATGACAGGGCAATCCCTCTCTCTGGAGAAACAGTG | |
| | | CACAGCCCCCTTTACTGGGCCAGACTGCAGACTTAAGTTCTCA | |
| | | GAGCAGGAGCCAAGGAAAGCAGAAGGAAAGGGCAACCGCAG | |
| | | GGGGAAGAAGGGGGGCAGGGTGCGATTCTGCCAGAAAGAGGG | |
| | | GGGCAACAGGGGAATCACAGAATGGGACATGAAGGGGAATTT | |
| | | CAGGCAGAGAAAGTGAAGCTCTGGCCATAAATAGGAGTCAGC | |
| | | TGCAGGGCTCACATTCCCAGCCTCACATCACTCACACCTTGCAT | |
| | | TTCACCCCTGCATCCCAGTCGCCCTGCAGCCTCACACAGATCCT | |
| | | GCACACACCCAGACAGCTGGCGCTCACACATTCACCGTTGGCC | |
| | | TGCCTCTGTTCACCCTCC | |
| SB07103 | CCL19 | GATGAATTAATGAACAGATACCTGAATAAAAGGATAGAAGGC | 148 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAGGTGAATTTTCTAACAGAGC | |
| | | TTATTGTGCGTCAGCCTCTAAGCTAAGGGCTTTACTCAGGGTTC | |
| | | CTCACTTAATCCTAAGAAGTAGATCTTCTACCATTCCCATTTTA | |
| | | CAAATAAGGACACTAAGACACAGAAACTGGAGAATTTTCACCT | |
| | | TGTCGCAGTTAGTAAGTGGTGGAATCTGAATCCCAAGCTAGGC | |
| | | GCTCTGTCTGCAGAGTCTCCCTCCTCTGCACCGTGACTACCCGT | |
| | | GCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07104 | CCL19 | GATGAATTAATGAACAGATACCTGAATAAAAGGATAGAAGGC | 149 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CTACTCATCACTAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07105 | CCL19 | GATGAATTAATGAACAGATACCTGAATAAAAGGATAGAAGGC | 150 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCTGCTAGTTGTCCAATAGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07106 | CCL19 | GATGAATTAATGAACAGATACCTGAATAAAAGGATAGAAGGC | 151 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACGTGTGTCATATAGAATTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07107 | CCL19 | GATGAATTAATGAACAGATACCTGAATAAAAGGATAGAAGGC | 152 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCAAACAGTCTA | |
| | | AGTCCTCAAATACCATTACTATTATTATGATTAATGAATGGAAA | |
| | | GAAGCTAAGTCAGAAATTTAAGAGGGAAATGTTCCCCCTCCCT | |
| | | CTCTCCATCCAAAAGAGGGTCTAGGGTTTGGGTGACGACGGGT | |
| | | GACTGGGCGTTTCACATTTAGTTTCTCTTTCAAGGCCTTCTCTTC | |
| | | TGACCTTTCTCATAAGGATGGGAAGTCACTTGTCCCCTCCCCCA | |
| | | TCGGCCGATTCCTTGAAATTTTAAGATTTGTCTGAATATGAGCT | |
| | | GGCCAGCCCCTCTCCTTAGAAATCCCCTCTTGGTTCCCAGGACT | |
| | | GAGAAAAGGGGGAAAATGAAGGGGGCTATTCCAAGCCAGTCT | |
| | | CCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07108 | CCL19 | GATGAATTAATGAACAGATACCTGAATAAAAGGATAGAAGGC | 153 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATACTCTACGGAAGTAGCTT | |
| | | GTTTAAAACCTATAGTATTTAAGAGGGAAATGTTCCCCCTCCCT | |
| | | CTCTCCATCCAAAAGAGGGTCTAGGGTTTGGGTGACGACGGGT | |
| | | GACTGGGCGTTTCACATTTAGTTTCTCTTTCAAGGCCTTCTCTTC | |
| | | TGACCTTTCTCATAAGGATGGGAAGTCACTTGTCCCCTCCCCCA | |
| | | TCGGCCGATTCCTTGAAATTTTAAGATTTGTCTGAATATGAGCT | |
| | | GGCCAGCCCCTCTCCTTAGAAATCCCCTCTTGGTTCCCAGGACT | |
| | | GAGAAAAGGGGGAAAATGAAGGGGGCTATTCCAAGCCAGTCT | |
| | | CCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07109 | CCL19 | GATGAATTAATGAACAGATACCTGAATAAAAGGATAGAAGGC | 154 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAGTTCTACTAGTACA | |
| | | AAGGTACCAGTAAAAGAGGGTCTAGGGTTTGGGTGACGACGG | |
| | | GTGACTGGGCGTTTCACATTTAGTTTCTCTTTCAAGGCCTTCTCT | |
| | | TCTGACCTTTCTCATAAGGATGGGAAGTCACTTGTCCCCTCCCC | |
| | | CATCGGCCGATTCCTTGAAATTTTAAGATTTGTCTGAATATGAG | |
| | | CTGGCCAGCCCCTCTCCTTAGAAATCCCCTCTTGGTTCCCAGGA | |
| | | CTGAGAAAAGGGGGAAAATGAAGGGGGCTATTCCAAGCCAGT | |
| | | CTCCCTCCAGCTGTTCCTTTGACCCAGGTGTTGGACATAGGAAA | |
| | | ACAGTAAATAGAGGAAGGGGAGGGTTTGCTGAGCCCCAGAGG | |
| | | CCTGAGATGTGGGATGGGGGTCCTGGGCCTCCTCCCAAGGTAC | |
| | | CTGGCCTTTCAGCTCTGCTTGCCCCAAGGGAAGGACTGGTTCCC | |
| | | ACATAGTGGGCAGGCACAGTGATGACCTTGGAGGCCACCCCAG | |
| | | GATCTCATGGTTGTCCTGAGGGTGCCAGGGAGCCTTCTTCACCA | |
| | | CCAGCCCAAGAAAGAAGCACCAGTGAGGACAAGGGATAAACC | |
| | | TAAGGAAGGGAGCACAGTTCCTCACTGTGGGCCCCACCTTTGA | |
| | | CCATCACTTCTGAACCCCAGCTTCATGGTTGATGATGCAAATGC | |
| | | CATCAATGGGATGACAGGGCAATCCCTCTCTCTGGAGAAACAG | |
| | | TGCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07110 | CCL19 | GATGAATTAATGAACAGATACCTGAATAAAAGGATAGAAGGC | 155 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTGAGTAAACTAACTTTCAACCGCTCTTTCTCT | |
| | | TCTGACCTTTCTCATAAGGATGGGAAGTCACTTGTCCCCTCCCC | |
| | | CATCGGCCGATTCCTTGAAATTTTAAGATTTGTCTGAATATGAG | |
| | | CTGGCCAGCCCCTCTCCTTAGAAATCCCCTCTTGGTTCCCAGGA | |
| | | CTGAGAAAAGGGGGAAAATGAAGGGGGCTATTCCAAGCCAGT | |
| | | CTCCCTCCAGCTGTTCCTTTGACCCAGGTGTTGGACATAGGAAA | |
| | | ACAGTAAATAGAGGAAGGGGAGGGTTTGCTGAGCCCCAGAGG | |
| | | CCTGAGATGTGGGATGGGGGTCCTGGGCCTCCTCCCAAGGTAC | |
| | | CTGGCCTTTCAGCTCTGCTTGCCCCAAGGGAAGGACTGGTTCCC | |
| | | ACATAGTGGGCAGGCACAGTGATGACCTTGGAGGCCACCCCAG | |
| | | GATCTCATGGTTGTCCTGAGGGTGCCAGGGAGCCTTCTTCACCA | |
| | | CCAGCCCAAGAAAGAAGCACCAGTGAGGACAAGGGATAAACC | |
| | | TAAGGAAGGGAGCACAGTTCCTCACTGTGGGCCCCACCTTTGA | |
| | | CCATCACTTCTGAACCCCAGCTTCATGGTTGATGATGCAAATGC | |
| | | CATCAATGGGATGACAGGGCAATCCCTCTCTCTGGAGAAACAG | |
| | | TGCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07111 | CCL19 | GATGAATTAATGAACAGATACCTGAATAAAAGGATAGAAGGC | 156 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTTCG | |
| | | TTACCATCTTTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07112 | CCL19 | GATGAATTAATGAACAGATACCTGAATAAAAGGATAGAAGGC | 157 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCAAAACACCGTTTTGCT | |
| | | GTAATATCATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07113 | CCL19 | GATGAATTAATGAACAGATACCTGAATAAAAGGATAGAAGGC | 158 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGCGCGTAGAACTTCGTAACATTAAGCTATTCCAAGCCAGTCTC | |
| | | CCTCCAGCTGTTCCTTTGACCCAGGTGTTGGACATAGGAAAAC | |
| | | AGTAAATAGAGGAAGGGGAGGGTTTGCTGAGCCCCAGAGGCC | |
| | | TGAGATGTGGGATGGGGGTCCTGGGCCTCCTCCCAAGGTACCT | |
| | | GGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07114 | CCL19 | GATGAATTAATGAACAGATACCTGAATAAAAGGATAGAAGGC | 159 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCAGATAACGCCGTCATTGTATCAGGTGTTGGACATAGGAAA | |
| | | ACAGTAAATAGAGGAAGGGGAGGGTTTGCTGAGCCCCAGAGG | |
| | | CCTGAGATGTGGGATGGGGGTCCTGGGCCTCCTCCCAAGGTAC | |
| | | CTGGCCTTTCAGCTCTGCTTGCCCCAAGGGAAGGACTGGTTCCC | |
| | | ACATAGTGGGCAGGCACAGTGATGACCTTGGAGGCCACCCCAG | |
| | | GATCTCATGGTTGTCCTGAGGGTGCCAGGGAGCCTTCTTCACCA | |
| | | CCAGCCCAAGAAAGAAGCACCAGTGAGGACAAGGGATAAACC | |
| | | TAAGGAAGGGAGCACAGTTCCTCACTGTGGGCCCCACCTTTGA | |
| | | CCATCACTTCTGAACCCCAGCTTCATGGTTGATGATGCAAATGC | |
| | | CATCAATGGGATGACAGGGCAATCCCTCTCTCTGGAGAAACAG | |
| | | TGCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07115 | CCL19 | GATGAATTAATGAACAGATACCTGAATAAAAGGATAGAAGGC | 160 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTTAACATCGTTCTCAGCTAGTTTGCTGAGCCCCAGAGGCCT | |
| | | GAGATGTGGGATGGGGGTCCTGGGCCTCCTCCCAAGGTACCTG | |
| | | GCCTTTCAGCTCTGCTTGCCCCAAGGGAAGGACTGGTTCCCAC | |
| | | ATAGTGGGCAGGCACAGTGATGACCTTGGAGGCCACCCCAGGA | |
| | | TCTCATGGTTGTCCTGAGGGTGCCAGGGAGCCTTCTTCACCACC | |
| | | AGCCCAAGAAAGAAGCACCAGTGAGGACAAGGGATAAACCTA | |
| | | AGGAAGGGAGCACAGTTCCTCACTGTGGGCCCCACCTTTGACC | |
| | | ATCACTTCTGAACCCCAGCTTCATGGTTGATGATGCAAATGCCA | |
| | | TCAATGGGATGACAGGGCAATCCCTCTCTCTGGAGAAACAGTG | |
| | | CACAGCCCCCTTTACTGGGCCAGACTGCAGACTTAAGTTCTCA | |
| | | GAGCAGGAGCCAAGGAAAGCAGAAGGAAAGGGCAACCGCAG | |
| | | GGGGAAGAAGGGGGGCAGGGTGCGATTCTGCCAGAAAGAGGG | |
| | | GGGCAACAGGGGAATCACAGAATGGGACATGAAGGGGAATTT | |
| | | CAGGCAGAGAAAGTGAAGCTCTGGCCATAAATAGGAGTCAGC | |
| | | TGCAGGGCTCACATTCCCAGCCTCACATCACTCACACCTTGCAT | |
| | | TTCACCCCTGCATCCCAGTCGCCCTGCAGCCTCACACAGATCCT | |
| | | GCACACACCCAGACAGCTGGCGCTCACACATTCACCGTTGGCC | |
| | | TGCCTCTGTTCACCCTCC | |
| SB07116 | CCL19 | GATGAATTAATGAACAGATACCTGAATAAAAGGATAGAAGGC | 161 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGATATACAGTGTTCAGCGTGTTACGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACACCCAGACAGCTGGCGCTCACACATTCACCGTTGGC | |
| | | CTGCCTCTGTTCACCCTCC | |
| SB07117 | CCL19 | GATGAATTAATGAACAGATACCTGAATAAAAGGATAGAAGGC | 162 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCG | |
| | | ACGTCTGTTAGTAGTATTACCCGTGTATTTCGGTCTTCGAGCAA | |
| | | TTACTTTATTGTCCTGAGGGTGCCAGGGAGCCTTCTTCACCACC | |
| | | AGCCCAAGAAAGAAGCACCAGTGAGGACAAGGGATAAACCTA | |
| | | AGGAAGGGAGCACAGTTCCTCACTGTGGGCCCCACCTTTGACC | |
| | | ATCACTTCTGAACCCCAGCTTCATGGTTGATGATGCAAATGCCA | |
| | | TCAATGGGATGACAGGGCAATCCCTCTCTCTGGAGAAACAGTG | |
| | | CACAGCCCCCTTTACTGGGCCAGACTGCAGACTTAAGTTCTCA | |
| | | GAGCAGGAGCCAAGGAAAGCAGAAGGAAAGGGCAACCGCAG | |
| | | GGGGAAGAAGGGGGGCAGGGTGCGATTCTGCCAGAAAGAGGG | |
| | | GGGCAACAGGGGAATCACAGAATGGGACATGAAGGGGAATTT | |
| | | CAGGCAGAGAAAGTGAAGCTCTGGCCATAAATAGGAGTCAGC | |
| | | TGCAGGGCTCACATTCCCAGCCTCACATCACTCACACCTTGCAT | |
| | | TTCACCCCTGCATCCCAGTCGCCCTGCAGCCTCACACAGATCCT | |
| | | GCACACACCCAGACAGCTGGCGCTCACACATTCACCGTTGGCC | |
| | | TGCCTCTGTTCACCCTCC | |
| SB07118 | CCL19 | GATGAATTAATGAACAGATACCTGAATAAAAGGATAGAAGGC | 163 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGTGCATAAAAAGAAATTCACCACGAGTA | |
| | | CCTATCTTGGTCTCGTTTGTTGCACTACTGCCAGAAAGAGGGGG | |
| | | GCAACAGGGGAATCACAGAATGGGACATGAAGGGGAATTTCA | |
| | | GGCAGAGAAAGTGAAGCTCTGGCCATAAATAGGAGTCAGCTG | |
| | | CAGGGCTCACATTCCCAGCCTCACATCACTCACACCTTGCATTT | |
| | | CACCCCTGCATCCCAGTCGCCCTGCAGCCTCACACAGATCCTGC | |
| | | ACACACCCAGACAGCTGGCGCTCACACATTCACCGTTGGCCTG | |
| | | CCTCTGTTCACCCTCC | |
| SB07119 | CCL19 | GATGAATTAATGAACAGATACCTGAATAAAAGGATAGAAGGC | 1 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATAAAAACTACCAACCAGTTATCATTTCT | |
| | | CTGTGTAATATCTGAAAGCTCTGGCCATAAATAGGAGTCAGCT | |
| | | GCAGGGCTCACATTCCCAGCCTCACATCACTCACACCTTGCATT | |
| | | TCACCCCTGCATCCCAGTCGCCCTGCAGCCTCACACAGATCCTG | |
| | | CACACACCCAGACAGCTGGCGCTCACACATTCACCGTTGGCCT | |
| | | GCCTCTGTTCACCCTCC | |
| SB07120 | CCL19 | GATGAATTAATGAACAGATACCTGAATAAAAGGATAGAAGGC | 2 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCGCAGAATATCGATATCTTCACACAGATCC | |
| | | TGCACACACCCAGACAGCTGGCGCTCACACATTCACCGTTGGC | |
| | | CTGCCTCTGTTCACCCTCC | |
| SB07121 | CCL19 | GATGAATTAATGAACAGATACCTGAATAAAAGGATAGAAGGC | 3 |
| | ablation | CAGGCACGGTGGCTCACGCCTGTAATCCCAACACTTTGGGAAG | |
| | variant | CGGAGGCAAGCAGATCATTTGAAGTCAGGAGTTCGAAACCAG | |
| | | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAAGAACAATGGCTAACAGAG | |
| | | CTTATTGTGCGTCAGCCTCTAAGCTAAGGGCTTTACTCAGGGTT | |
| | | CCTCACTTAATCCTAAGAAGTAGATCTTCTACCATTCCCATTTT | |
| | | ACAAATAAGGACACTAAGACACAGAAACTGGAGAATTTTCACC | |
| | | TTGTCGCAGTTAGTAAGTGGTGGAATCTGAATCCCAAGCTAGG | |
| | | CGCTCTGTCTGCAGAGTCTCCCTCCTCTGCACCGTGACTACCCG | |
| | | TGCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGAGAAAAGGGGGAAAATGAAGGGGGCTATTCCAAGCCAGTC | |
| | | TCCCTCCAGCTGTTCCTTTGACCCAGGTGTTGGACATAGGAAAA | |
| | | CAGTAAATAGAGGAAGGGGAGGGTTTGCTGAGCCCCAGAGGC | |
| | | CTGAGATGTGGGATGGGGGTCCTGGGCCTCCTCCCAAGGTACC | |
| | | TGGCCTTTCAGCTCTGCTTGCCCCAAGGGAAGGACTGGTTCCCA | |
| | | CATAGTGGGCAGGCACAGTGATGACCTTGGAGGCCACCCCAGG | |
| | | ATCTCATGGTTGTCCTGAGGGTGCCAGGGAGCCTTCTTCACCAC | |
| | | CAGCCCAAGAAAGAAGCACCAGTGAGGACAAGGGATAAACCT | |
| | | AAGGAAGGGAGCACAGTTCCTCACTGTGGGCCCCACCTTTGAC | |
| | | CATCACTTCTGAACCCCAGCTTCATGGTTGATGATGCAAATGCC | |
| | | ATCAATGGGATGACAGGGCAATCCCTCTCTCTGGAGAAACAGT | |
| | | GCACAGCCCCCTTTACTGGGCCAGACTGCAGACTTAAGTTCTC | |
| | | AGAGCAGGAGCCAAGGAAAGCAGAAGGAAAGGGCAACCGCA | |
| | | GGGGGAAGAAGGGGGGCAGGGTGCGATTCTGCCAGAAAGAGG | |
| | | GGGGCAACAGGGGAATCACAGAATGGGACATGAAGGGGAATT | |
| | | TCAGGCAGAGAAAGTGAAGCTCTGGCCATAAATAGGAGTCAG | |
| | | CTGCAGGGCTCACATTCCCAGCCTCACATCACTCACACCTTGCA | |
| | | TTTCACCCCTGCATCCCAGTCGCCCTGCAGCCTCACACAGATCC | |
| | | TGCACACCGAATAGCACCTATAGCTCACACATTCACCGTTGGC | |
| | | CTGCCTCTGTTCACCCTCC | |
| SB09386 | IDO1 | GCTCTTCTAAAAATATGCGAAATGAGGTTTTTAGGGAGGTGTA | 4 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAACTTACCTACTAAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09387 | IDO1 | GCTCTTCTAAAAATATGCGAAATGAGGTTTTTAGGGAGGTGTA | 6 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGTAATTCGTCCGATAG | |
| | | ATTGAGACGGAGTCTCACTCTGCTGCCCAGGCTGGAGTGCAAT | |
| | | GGTGCAATCTTGGCTCACTGCAACCTCTGCCTCTCGGGCTCAAG | |
| | | CAATTCTCCTGCCTCAGCCTCCCGAGTAGCTGGGATTACAGGC | |
| | | ACATGCCACCATGCCCAGCTAATTTTTGTATTTTCAATAGAGAC | |
| | | TGGGTTTCACCATGTTGCCCAGGCTGGTCTTCAACTCCTGACCT | |
| | | CAAGTGATCCGCCCTCCTCAGCTTCCCAAAGTGCTGGGATTAC | |
| | | AGGCGTGAGCCACCACACCGGGGGGTAGGATAGATTTAGTGA | |
| | | GATGACTGGATAAACGGAATCAAGAAAAAGCTTTGTCAAAAA | |
| | | CTTATGCTTCTTAAAAACTTAATCCTGGGACAGAATCATCTAAA | |
| | | ACGTTGTTCCATGTCTTCACTTTGACTCACCCATAAAAACTTCA | |
| | | AGTACAAAGAATGAAAAATAACCACATATTTTCTAATGCTCAA | |
| | | TATTTTATTTGTAGTGTTGTTTTCTATATAGATATGATAGGTCTT | |
| | | CATGATTTTTTGTTTGTTTTCCTTGAACTGATTCCCAAAGTATTA | |
| | | GCCTCATGAATCATGTAGTCATAAGAAACACAGTCATTGTATT | |
| | | CTCTTTGCTGTATAATTTTGGTTTCAGTTTTCCTTACATTTCCTA | |
| | | TTCAAGGAACATTTTCCTGTAAAATGACAGGTTGAAGAAAACA | |
| | | GCCATAATTTAGTAGAGAATAGCGCGAGAGCTATTCTAGACTG | |
| | | TAACGAAAGCCATATGCTATCACAATTTAATTTATTTCAAGTAC | |
| | | TAATAAGCTGATGACAAAACAGCGATGTCTTTTAGTTTACTCAC | |
| | | ACGAACTATTTCTCTTTTCTCCTTTTGATCATCTAGAGGAACGG | |
| | | GCAACTTGGTTTCTTCTTTAGCTTCCTTGTTCTCATTAAGATTGA | |
| | | ACAATGCCTCTAAAGTGAACCACAGACTTGCATGCAAGCTGAA | |
| | | AACCTTTACCAAATGCAGTCTTAATTTGTACTTTGAGAAAAACA | |
| | | TTTTCAAGGTATTTTATCCTTTTCTCCAACTTTTGACATATTACA | |
| | | AAGTACCCAAATATGCCAGACTGTTGCCTCATCAGCCCCCCGC | |
| | | AGTCAGGTACAGTTAGATGCAAGGCAATCTTCCTAAAAGTTAC | |
| | | TTATTAGAGATGTGAGAAGGGCAAATGCTATCATTGGAAAAAC | |
| | | TGACAAAAGTCCCAATAGGAAAAATAAGGAAGTGGAGAGTTA | |
| | | CTATGTTTCTAATTTTTCATGTGCTTCTATTTTTTTCCTACTTCA | |
| | | GAGCCATTGACTAATAGTTGAGTATAACACAGGTTGTGTTTCC | |
| | | GGGCTGCTGAAACATGACACTAATATTTTCAAAGAACTGTGGA | |
| | | AGCCTAAAAGGAAGCCAATGAGAAATAACTAAATGAGAGTTT | |
| | | AGGACTGCAGCCTTCATTTTCATTCAAAGATTTAAAAGTTTCCA | |
| | | TAAAGTAAAATGTTCTTCTCCGGCCACCTGTTTTCATAGTTCTG | |
| | | TGTTTTCCTTCAGGCCTTTCTGGCTTCCTATATGGCAGTAAGAA | |
| | | AATGATGTGCTTAATGATTACAAATTTCATATGGAATACGAAC | |
| | | TTTCAGTTTGTACATATGATGCACAGAGATGCTTTTGTGGTTTT | |
| | | ATTGGTTTTCATATTACAAACAAAGAAACTAGAAAATGAAACC | |
| | | ATTCCAAAAGTGGAAGTAATTTCTCACTGCCCCTGTGATAAACT | |
| | | GTGGTCACTGGCTGTGGCAGCAACTATTATAAGATGCTCTGAA | |
| | | AACTCTTCAGACACTGAGGGGCACCAGAGGAGCAGACTACAA | |
| | | GA | |
| SB09388 | IDO1 | GCTCTTCTAAAAATATGCGAAATGAGGTTTTTAGGGAGGTGTA | 7 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAATTCAGACGACAAACCATTCTTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09389 | IDO1 | GCTCTTCTAAAAATATGCGAAATGAGGTTTTTAGGGAGGTGTA | 8 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCT | |
| | | TCTAAGTCCAATTCACGACAAGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09390 | IDO1 | GCTCTTCTAAAAATATGCGAAATGAGGTTTTTAGGGAGGTGTA | 9 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCGTTGAAGCTTGGCACA | |
| | | TGCCACCATGCCCAGCTAATTTTTGTATTTTCAATAGAGACTGG | |
| | | GTTTCACCATGTTGCCCAGGCTGGTCTTCAACTCCTGACCTCAA | |
| | | GTGATCCGCCCTCCTCAGCTTCCCAAAGTGCTGGGATTACAGG | |
| | | CGTGAGCCACCACACCGGGGGGTAGGATAGATTTAGTGAGATG | |
| | | ACTGGATAAACGGAATCAAGAAAAAGCTTTGTCAAAAACTTAT | |
| | | GCTTCTTAAAAACTTAATCCTGGGACAGAATCATCTAAAACGT | |
| | | TGTTCCATGTCTTCACTTTGACTCACCCATAAAAACTTCAAGTA | |
| | | CAAAGAATGAAAAATAACCACATATTTTCTAATGCTCAATATT | |
| | | TTATTTGTAGTGTTGTTTTCTATATAGATATGATAGGTCTTCATG | |
| | | ATTTTTTGTTTGTTTTCCTTGAACTGATTCCCAAAGTATTAGCCT | |
| | | CATGAATCATGTAGTCATAAGAAACACAGTCATTGTATTCTCTT | |
| | | TGCTGTATAATTTTGGTTTCAGTTTTCCTTACATTTCCTATTCAA | |
| | | GGAACATTTTCCTGTAAAATGACAGGTTGAAGAAAACAGCCAT | |
| | | AATTTAGTAGAGAATAGCGCGAGAGCTATTCTAGACTGTAACG | |
| | | AAAGCCATATGCTATCACAATTTAATTTATTTCAAGTACTAATA | |
| | | AGCTGATGACAAAACAGCGATGTCTTTTAGTTTACTCACACGA | |
| | | ACTATTTCTCTTTTCTCCTTTTGATCATCTAGAGGAACGGGCAA | |
| | | CTTGGTTTCTTCTTTAGCTTCCTTGTTCTCATTAAGATTGAACAA | |
| | | TGCCTCTAAAGTGAACCACAGACTTGCATGCAAGCTGAAAACC | |
| | | TTTACCAAATGCAGTCTTAATTTGTACTTTGAGAAAAACATTTT | |
| | | CAAGGTATTTTATCCTTTTCTCCAACTTTTGACATATTACAAAG | |
| | | TACCCAAATATGCCAGACTGTTGCCTCATCAGCCCCCCGCAGTC | |
| | | AGGTACAGTTAGATGCAAGGCAATCTTCCTAAAAGTTACTTAT | |
| | | TAGAGATGTGAGAAGGGCAAATGCTATCATTGGAAAAACTGAC | |
| | | AAAAGTCCCAATAGGAAAAATAAGGAAGTGGAGAGTTACTAT | |
| | | GTTTCTAATTTTTCATGTGCTTCTATTTTTTTCCTACTTCAGAGC | |
| | | CATTGACTAATAGTTGAGTATAACACAGGTTGTGTTTCCGGGCT | |
| | | GCTGAAACATGACACTAATATTTTCAAAGAACTGTGGAAGCCT | |
| | | AAAAGGAAGCCAATGAGAAATAACTAAATGAGAGTTTAGGAC | |
| | | TGCAGCCTTCATTTTCATTCAAAGATTTAAAAGTTTCCATAAAG | |
| | | TAAAATGTTCTTCTCCGGCCACCTGTTTTCATAGTTCTGTGTTTT | |
| | | CCTTCAGGCCTTTCTGGCTTCCTATATGGCAGTAAGAAAATGAT | |
| | | GTGCTTAATGATTACAAATTTCATATGGAATACGAACTTTCAGT | |
| | | TTGTACATATGATGCACAGAGATGCTTTTGTGGTTTTATTGGTT | |
| | | TTCATATTACAAACAAAGAAACTAGAAAATGAAACCATTCCAA | |
| | | AAGTGGAAGTAATTTCTCACTGCCCCTGTGATAAACTGTGGTC | |
| | | ACTGGCTGTGGCAGCAACTATTATAAGATGCTCTGAAAACTCT | |
| | | TCAGACACTGAGGGGCACCAGAGGAGCAGACTACAAGA | |
| SB09391 | IDO1 | GCTCTTCTAAAAATATGCGAAATGAGGTTTTTAGGGAGGTGTA | 10 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | AGTCGTCAGACTCAATTATTACTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09392 | IDO1 | GCTCTTCTAAAAATATGCGAAATGAGGTTTTTAGGGAGGTGTA | 11 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAATCCCTAGCGATC | |
| | | GAAGTTGATAAAACCTAAGTTTTGTAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09393 | IDO1 | GCTCTTCTAAAAATATGCGAAATGAGGTTTTTAGGGAGGTGTA | 12 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCGCCTTCATAAGG | |
| | | CGTGAGCCACCACACCGGGGGGTAGGATAGATTTAGTGAGATG | |
| | | ACTGGATAAACGGAATCAAGAAAAAGCTTTGTCAAAAACTTAT | |
| | | GCTTCTTAAAAACTTAATCCTGGGACAGAATCATCTAAAACGT | |
| | | TGTTCCATGTCTTCACTTTGACTCACCCATAAAAACTTCAAGTA | |
| | | CAAAGAATGAAAAATAACCACATATTTTCTAATGCTCAATATT | |
| | | TTATTTGTAGTGTTGTTTTCTATATAGATATGATAGGTCTTCATG | |
| | | ATTTTTTGTTTGTTTTCCTTGAACTGATTCCCAAAGTATTAGCCT | |
| | | CATGAATCATGTAGTCATAAGAAACACAGTCATTGTATTCTCTT | |
| | | TGCTGTATAATTTTGGTTTCAGTTTTCCTTACATTTCCTATTCAA | |
| | | GGAACATTTTCCTGTAAAATGACAGGTTGAAGAAAACAGCCAT | |
| | | AATTTAGTAGAGAATAGCGCGAGAGCTATTCTAGACTGTAACG | |
| | | AAAGCCATATGCTATCACAATTTAATTTATTTCAAGTACTAATA | |
| | | AGCTGATGACAAAACAGCGATGTCTTTTAGTTTACTCACACGA | |
| | | ACTATTTCTCTTTTCTCCTTTTGATCATCTAGAGGAACGGGCAA | |
| | | CTTGGTTTCTTCTTTAGCTTCCTTGTTCTCATTAAGATTGAACAA | |
| | | TGCCTCTAAAGTGAACCACAGACTTGCATGCAAGCTGAAAACC | |
| | | TTTACCAAATGCAGTCTTAATTTGTACTTTGAGAAAAACATTTT | |
| | | CAAGGTATTTTATCCTTTTCTCCAACTTTTGACATATTACAAAG | |
| | | TACCCAAATATGCCAGACTGTTGCCTCATCAGCCCCCCGCAGTC | |
| | | AGGTACAGTTAGATGCAAGGCAATCTTCCTAAAAGTTACTTAT | |
| | | TAGAGATGTGAGAAGGGCAAATGCTATCATTGGAAAAACTGAC | |
| | | AAAAGTCCCAATAGGAAAAATAAGGAAGTGGAGAGTTACTAT | |
| | | GTTTCTAATTTTTCATGTGCTTCTATTTTTTTCCTACTTCAGAGC | |
| | | CATTGACTAATAGTTGAGTATAACACAGGTTGTGTTTCCGGGCT | |
| | | GCTGAAACATGACACTAATATTTTCAAAGAACTGTGGAAGCCT | |
| | | AAAAGGAAGCCAATGAGAAATAACTAAATGAGAGTTTAGGAC | |
| | | TGCAGCCTTCATTTTCATTCAAAGATTTAAAAGTTTCCATAAAG | |
| | | TAAAATGTTCTTCTCCGGCCACCTGTTTTCATAGTTCTGTGTTTT | |
| | | CCTTCAGGCCTTTCTGGCTTCCTATATGGCAGTAAGAAAATGAT | |
| | | GTGCTTAATGATTACAAATTTCATATGGAATACGAACTTTCAGT | |
| | | TTGTACATATGATGCACAGAGATGCTTTTGTGGTTTTATTGGTT | |
| | | TTCATATTACAAACAAAGAAACTAGAAAATGAAACCATTCCAA | |
| | | AAGTGGAAGTAATTTCTCACTGCCCCTGTGATAAACTGTGGTC | |
| | | ACTGGCTGTGGCAGCAACTATTATAAGATGCTCTGAAAACTCT | |
| | | TCAGACACTGAGGGGCACCAGAGGAGCAGACTACAAGA | |
| SB09394 | IDO1 | GCTCTTCTAAAAATATGCGAAATGAGGTTTTTAGGGAGGTGTA | 13 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | CTCGCTAATAGGAGTAAGATACATATAGATATGATAGGTCTTC | |
| | | ATGATTTTTTGTTTGTTTTCCTTGAACTGATTCCCAAAGTATTAG | |
| | | CCTCATGAATCATGTAGTCATAAGAAACACAGTCATTGTATTCT | |
| | | CTTTGCTGTATAATTTTGGTTTCAGTTTTCCTTACATTTCCTATT | |
| | | CAAGGAACATTTTCCTGTAAAATGACAGGTTGAAGAAAACAGC | |
| | | CATAATTTAGTAGAGAATAGCGCGAGAGCTATTCTAGACTGTA | |
| | | ACGAAAGCCATATGCTATCACAATTTAATTTATTTCAAGTACTA | |
| | | ATAAGCTGATGACAAAACAGCGATGTCTTTTAGTTTACTCACA | |
| | | CGAACTATTTCTCTTTTCTCCTTTTGATCATCTAGAGGAACGGG | |
| | | CAACTTGGTTTCTTCTTTAGCTTCCTTGTTCTCATTAAGATTGAA | |
| | | CAATGCCTCTAAAGTGAACCACAGACTTGCATGCAAGCTGAAA | |
| | | ACCTTTACCAAATGCAGTCTTAATTTGTACTTTGAGAAAAACAT | |
| | | TTTCAAGGTATTTTATCCTTTTCTCCAACTTTTGACATATTACAA | |
| | | AGTACCCAAATATGCCAGACTGTTGCCTCATCAGCCCCCCGCA | |
| | | GTCAGGTACAGTTAGATGCAAGGCAATCTTCCTAAAAGTTACT | |
| | | TATTAGAGATGTGAGAAGGGCAAATGCTATCATTGGAAAAACT | |
| | | GACAAAAGTCCCAATAGGAAAAATAAGGAAGTGGAGAGTTAC | |
| | | TATGTTTCTAATTTTTCATGTGCTTCTATTTTTTTCCTACTTCAG | |
| | | AGCCATTGACTAATAGTTGAGTATAACACAGGTTGTGTTTCCG | |
| | | GGCTGCTGAAACATGACACTAATATTTTCAAAGAACTGTGGAA | |
| | | GCCTAAAAGGAAGCCAATGAGAAATAACTAAATGAGAGTTTA | |
| | | GGACTGCAGCCTTCATTTTCATTCAAAGATTTAAAAGTTTCCAT | |
| | | AAAGTAAAATGTTCTTCTCCGGCCACCTGTTTTCATAGTTCTGT | |
| | | GTTTTCCTTCAGGCCTTTCTGGCTTCCTATATGGCAGTAAGAAA | |
| | | ATGATGTGCTTAATGATTACAAATTTCATATGGAATACGAACTT | |
| | | TCAGTTTGTACATATGATGCACAGAGATGCTTTTGTGGTTTTAT | |
| | | TGGTTTTCATATTACAAACAAAGAAACTAGAAAATGAAACCAT | |
| | | TCCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09395 | IDO1 | GCTCTTCTAAAAATATGCGAAATGAGGTTTTTAGGGAGGTGTA | 14 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | TCTGCTGCAAGACCTATACTATAACTGATTCCCAAAGTATTAGC | |
| | | CTCATGAATCATGTAGTCATAAGAAACACAGTCATTGTATTCTC | |
| | | TTTGCTGTATAATTTTGGTTTCAGTTTTCCTTACATTTCCTATTC | |
| | | AAGGAACATTTTCCTGTAAAATGACAGGTTGAAGAAAACAGCC | |
| | | ATAATTTAGTAGAGAATAGCGCGAGAGCTATTCTAGACTGTAA | |
| | | CGAAAGCCATATGCTATCACAATTTAATTTATTTCAAGTACTAA | |
| | | TAAGCTGATGACAAAACAGCGATGTCTTTTAGTTTACTCACAC | |
| | | GAACTATTTCTCTTTTCTCCTTTTGATCATCTAGAGGAACGGGC | |
| | | AACTTGGTTTCTTCTTTAGCTTCCTTGTTCTCATTAAGATTGAAC | |
| | | AATGCCTCTAAAGTGAACCACAGACTTGCATGCAAGCTGAAAA | |
| | | CCTTTACCAAATGCAGTCTTAATTTGTACTTTGAGAAAAACATT | |
| | | TTCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09396 | IDO1 | GCTCTTCTAAAAATATGCGAAATGAGGTTTTTAGGGAGGTGTA | 15 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATACCACATTGCTATAGTGCTGTATAATTTCCTATTC | |
| | | AAGGAACATTTTCCTGTAAAATGACAGGTTGAAGAAAACAGCC | |
| | | ATAATTTAGTAGAGAATAGCGCGAGAGCTATTCTAGACTGTAA | |
| | | CGAAAGCCATATGCTATCACAATTTAATTTATTTCAAGTACTAA | |
| | | TAAGCTGATGACAAAACAGCGATGTCTTTTAGTTTACTCACAC | |
| | | GAACTATTTCTCTTTTCTCCTTTTGATCATCTAGAGGAACGGGC | |
| | | AACTTGGTTTCTTCTTTAGCTTCCTTGTTCTCATTAAGATTGAAC | |
| | | AATGCCTCTAAAGTGAACCACAGACTTGCATGCAAGCTGAAAA | |
| | | CCTTTACCAAATGCAGTCTTAATTTGTACTTTGAGAAAAACATT | |
| | | TTCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09397 | IDO1 | GCTCTTCTAAAAATATGCGAAATGAGGTTTTTAGGGAGGTGTA | 16 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTTGCGTACCAGAATATTTAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09398 | IDO1 | GCTCTTCTAAAAATATGCGAAATGAGGTTTTTAGGGAGGTGTA 1 | |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTTGGTCACTATCACGTATATACCAT | |
| | | CAGGTACAGTTAGATGCAAGGCAATCTTCCTAAAAGTTACTTA | |
| | | TTAGAGATGTGAGAAGGGCAAATGCTATCATTGGAAAAACTGA | |
| | | CAAAAGTCCCAATAGGAAAAATAAGGAAGTGGAGAGTTACTA | |
| | | TGTTTCTAATTTTTCATGTGCTTCTATTTTTTTCCTACTTCAGAG | |
| | | CCATTGACTAATAGTTGAGTATAACACAGGTTGTGTTTCCGGGC | |
| | | TGCTGAAACATGACACTAATATTTTCAAAGAACTGTGGAAGCC | |
| | | TAAAAGGAAGCCAATGAGAAATAACTAAATGAGAGTTTAGGA | |
| | | CTGCAGCCTTCATTTTCATTCAAAGATTTAAAAGTTTCCATAAA | |
| | | GTAAAATGTTCTTCTCCGGCCACCTGTTTTCATAGTTCTGTGTTT | |
| | | TCCTTCAGGCCTTTCTGGCTTCCTATATGGCAGTAAGAAAATGA | |
| | | TGTGCTTAATGATTACAAATTTCATATGGAATACGAACTTTCAG | |
| | | TTTGTACATATGATGCACAGAGATGCTTTTGTGGTTTTATTGGT | |
| | | TTTCATATTACAAACAAAGAAACTAGAAAATGAAACCATTCCA | |
| | | AAAGTGGAAGTAATTTCTCACTGCCCCTGTGATAAACTGTGGT | |
| | | CACTGGCTGTGGCAGCAACTATTATAAGATGCTCTGAAAACTC | |
| | | TTCAGACACTGAGGGGCACCAGAGGAGCAGACTACAAGA | |
| SB09399 | IDO1 | GCTCTTCTAAAAATATGCGAAATGAGGTTTTTAGGGAGGTGTA | 18 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGACGAGTTCGATAATACACTGTTACTA | |
| | | TGTTTCTAATTTTTCATGTGCTTCTATTTTTTTCCTACTTCAGAG | |
| | | CCATTGACTAATAGTTGAGTATAACACAGGTTGTGTTTCCGGGC | |
| | | TGCTGAAACATGACACTAATATTTTCAAAGAACTGTGGAAGCC | |
| | | TAAAAGGAAGCCAATGAGAAATAACTAAATGAGAGTTTAGGA | |
| | | CTGCAGCCTTCATTTTCATTCAAAGATTTAAAAGTTTCCATAAA | |
| | | GTAAAATGTTCTTCTCCGGCCACCTGTTTTCATAGTTCTGTGTTT | |
| | | TCCTTCAGGCCTTTCTGGCTTCCTATATGGCAGTAAGAAAATGA | |
| | | TGTGCTTAATGATTACAAATTTCATATGGAATACGAACTTTCAG | |
| | | TTTGTACATATGATGCACAGAGATGCTTTTGTGGTTTTATTGGT | |
| | | TTTCATATTACAAACAAAGAAACTAGAAAATGAAACCATTCCA | |
| | | AAAGTGGAAGTAATTTCTCACTGCCCCTGTGATAAACTGTGGT | |
| | | CACTGGCTGTGGCAGCAACTATTATAAGATGCTCTGAAAACTC | |
| | | TTCAGACACTGAGGGGCACCAGAGGAGCAGACTACAAGA | |
| SB09400 | IDO1 | GCTCTTCTAAAAATATGCGAAATGAGGTTTTTAGGGAGGTGTA | 19 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGAATACTGGTGCTTCAATTACTTCAG | |
| | | AGCCATTGACTAATAGTTGAGTATAACACAGGTTGTGTTTCCG | |
| | | GGCTGCTGAAACATGACACTAATATTTTCAAAGAACTGTGGAA | |
| | | GCCTAAAAGGAAGCCAATGAGAAATAACTAAATGAGAGTTTA | |
| | | GGACTGCAGCCTTCATTTTCATTCAAAGATTTAAAAGTTTCCAT | |
| | | AAAGTAAAATGTTCTTCTCCGGCCACCTGTTTTCATAGTTCTGT | |
| | | GTTTTCCTTCAGGCCTTTCTGGCTTCCTATATGGCAGTAAGAAA | |
| | | ATGATGTGCTTAATGATTACAAATTTCATATGGAATACGAACTT | |
| | | TCAGTTTGTACATATGATGCACAGAGATGCTTTTGTGGTTTTAT | |
| | | TGGTTTTCATATTACAAACAAAGAAACTAGAAAATGAAACCAT | |
| | | TCCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09401 | IDO1 | GCTCTTCTAAAAATATGCGAAATGAGGTTTTTAGGGAGGTGTA | 20 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCCCGATAGAAAGAATAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09402 | IDO1 | GCTCTTCTAAAAATATGCGAAATGAGGTTTTTAGGGAGGTGTA | 21 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGTGTCTGTATAAAGATAACTAAATGAGAGTTTAGG | |
| | | ACTGCAGCCTTCATTTTCATTCAAAGATTTAAAAGTTTCCATAA | |
| | | AGTAAAATGTTCTTCTCCGGCCACCTGTTTTCATAGTTCTGTGT | |
| | | TTTCCTTCAGGCCTTTCTGGCTTCCTATATGGCAGTAAGAAAAT | |
| | | GATGTGCTTAATGATTACAAATTTCATATGGAATACGAACTTTC | |
| | | AGTTTGTACATATGATGCACAGAGATGCTTTTGTGGTTTTATTG | |
| | | GTTTTCATATTACAAACAAAGAAACTAGAAAATGAAACCATTC | |
| | | CAAAAGTGGAAGTAATTTCTCACTGCCCCTGTGATAAACTGTG | |
| | | GTCACTGGCTGTGGCAGCAACTATTATAAGATGCTCTGAAAAC | |
| | | TCTTCAGACACTGAGGGGCACCAGAGGAGCAGACTACAAGA | |
| SB09403 | IDO1 | GCTCTTCTAAAAATATGCGAAATGAGGTTTTTAGGGAGGTGTA | 22 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTGTTAAGCATACT | |
| | | AAACTGTCATATTACAAACAAAGAAACTAGAAAATGAAACCAT | |
| | | TCCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09404 | IDO1 | GCTCTTCTAAAAATATGCGAAATGAGGTTTTTAGGGAGGTGTA | 23 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGTTTCGAGCGACGCTTAATATTC | |
| | | CAAAAGTGGAAGTAATTTCTCACTGCCCCTGTGATAAACTGTG | |
| | | GTCACTGGCTGTGGCAGCAACTATTATAAGATGCTCTGAAAAC | |
| | | TCTTCAGACACTGAGGGGCACCAGAGGAGCAGACTACAAGA | |
| SB09405 | IDO1 | GCTCTTCTAAAAATATGCGAAATGAGGTTTTTAGGGAGGTGTA | 24 |
| | ablation | GGTATGGCTGAAGAAAATCAAGGTGAATGAAGACAAGATCAA | |
| | variant | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ATTCTCCTGCCTCAGCCTCCCGAGTAGCTGGGATTACAGGCAC | |
| | | ATGCCACCATGCCCAGCTAATTTTTGTATTTTCAATAGAGACTG | |
| | | GGTTTCACCATGTTGCCCAGGCTGGTCTTCAACTCCTGACCTCA | |
| | | AGTGATCCGCCCTCCTCAGCTTCCCAAAGTGCTGGGATTACAG | |
| | | GCGTGAGCCACCACACCGGGGGGTAGGATAGATTTAGTGAGAT | |
| | | GACTGGATAAACGGAATCAAGAAAAAGCTTTGTCAAAAACTTA | |
| | | TGCTTCTTAAAAACTTAATCCTGGGACAGAATCATCTAAAACG | |
| | | TTGTTCCATGTCTTCACTTTGACTCACCCATAAAAACTTCAAGT | |
| | | ACAAAGAATGAAAAATAACCACATATTTTCTAATGCTCAATAT | |
| | | TTTATTTGTAGTGTTGTTTTCTATATAGATATGATAGGTCTTCAT | |
| | | GATTTTTTGTTTGTTTTCCTTGAACTGATTCCCAAAGTATTAGCC | |
| | | TCATGAATCATGTAGTCATAAGAAACACAGTCATTGTATTCTCT | |
| | | TTGCTGTATAATTTTGGTTTCAGTTTTCCTTACATTTCCTATTCA | |
| | | AGGAACATTTTCCTGTAAAATGACAGGTTGAAGAAAACAGCCA | |
| | | TAATTTAGTAGAGAATAGCGCGAGAGCTATTCTAGACTGTAAC | |
| | | GAAAGCCATATGCTATCACAATTTAATTTATTTCAAGTACTAAT | |
| | | AAGCTGATGACAAAACAGCGATGTCTTTTAGTTTACTCACACG | |
| | | AACTATTTCTCTTTTCTCCTTTTGATCATCTAGAGGAACGGGCA | |
| | | ACTTGGTTTCTTCTTTAGCTTCCTTGTTCTCATTAAGATTGAACA | |
| | | ATGCCTCTAAAGTGAACCACAGACTTGCATGCAAGCTGAAAAC | |
| | | CTTTACCAAATGCAGTCTTAATTTGTACTTTGAGAAAAACATTT | |
| | | TCAAGGTATTTTATCCTTTTCTCCAACTTTTGACATATTACAAA | |
| | | GTACCCAAATATGCCAGACTGTTGCCTCATCAGCCCCCCGCAG | |
| | | TCAGGTACAGTTAGATGCAAGGCAATCTTCCTAAAAGTTACTT | |
| | | ATTAGAGATGTGAGAAGGGCAAATGCTATCATTGGAAAAACTG | |
| | | ACAAAAGTCCCAATAGGAAAAATAAGGAAGTGGAGAGTTACT | |
| | | ATGTTTCTAATTTTTCATGTGCTTCTATTTTTTTCCTACTTCAGA | |
| | | GCCATTGACTAATAGTTGAGTATAACACAGGTTGTGTTTCCGG | |
| | | GCTGCTGAAACATGACACTAATATTTTCAAAGAACTGTGGAAG | |
| | | CCTAAAAGGAAGCCAATGAGAAATAACTAAATGAGAGTTTAG | |
| | | GACTGCAGCCTTCATTTTCATTCAAAGATTTAAAAGTTTCCATA | |
| | | AAGTAAAATGTTCTTCTCCGGCCACCTGTTTTCATAGTTCTGTG | |
| | | TTTTCCTTCAGGCCTTTCTGGCTTCCTATATGGCAGTAAGAAAA | |
| | | TGATGTGCTTAATGATTACAAATTTCATATGGAATACGAACTTT | |
| | | CAGTTTGTACATATGATGCACAGAGATGCTTTTGTGGTTTTATT | |
| | | GGTTTTCATATTACAAACAAAGAAACTAGAAAATGAAACCATT | |
| | | CCATAGATAGTACGGGTTCCATACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB09407 | UBD1 | TCTCCTTCACCTTCCACCATGAGTAAAAGCTTCTTGAGGCCTCT | 25 |
| | ablation | TACCTACTAGGTTAAGGTGCCATACTTCTTGTATAGTTGGAGGA | |
| | variant | ACTATGAATCAATTAAACTTTTTTTCCTTATAAATTACCCAGCC | |
| | | TCAAGTATTCCTTTATAGTATGCAAAAGAGACTAAAACAGCAA | |
| | | ATGAAAAGAGATCTGTCTCTGAAAGAACTTATTGTGACATAGA | |
| | | GAGAGACAGAAAAAAGTCAGATTGACACTGGGTAAGAAGCAA | |
| | | GGAGGTCAGGTCCCAAGATGAAGTCCTGCCTGTGGTCAGCAAA | |
| | | GGGGCACCAAGGTGTCTGGGACAGTCCTGGCCCTGGCTTTGGG | |
| | | CAGGGAGGGAATTTCCCATAGGAAGGGAAGAGTAAAGAGAGA | |
| | | GAGAGAGGTCAGAGTCCAGGTTTGTTGTTCATATGTTTTCTTGA | |
| | | AAGGGCACTATTTCCCAGAATCCAGGTCATCTCTGGGTAGGGA | |
| | | ATCCCCTGAATTAGTTTTTTCTGAGAGTATATTAGATTTGCCCT | |
| | | CACAGTAACCCCATGCTGGAAATACCCAACTCCAGTAGCACTT | |
| | | TTCTCCCTGCCTTTTCCCACTGAAATAACATAATAATATAGGAA | |
| | | GAATACAAGGACTATAGAAATACAGATTAGTGTTTGAACCCTT | |
| | | GCTTACCAGCTACTACTAATATGATTGTGGATGAGGTAGCTTCT | |
| | | TACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09408 | UBD1 | TCTCCTTCACCTTCCACCATGAGTAAAAGCTTCTTGAGGCCTCA | 26 |
| | ablation | CCAGAAGCAGATGCTGGTGCCATACTTCTTGTATAGTTGGAGG | |
| | variant | AACTATGAATCAATTAAACACTCGAATTCAGAAAATTACCCAG | |
| | | CCTCAAGTATTCCTTTATAGTATGCAAAAGAGACTAAAACAGC | |
| | | AAATGAAAAGAGATCTGTCTCTGAAAGAACTTATTGTGACATA | |
| | | GAGAGAGACAGAAAAAAGTCAGATTGACACTGGGTAAGAAGC | |
| | | AAGGAGGTCAGGTCCCAAGATGAAGTCCTGCCTGTGGTCAGCA | |
| | | AAGGGGCACCAAGGTGTCTGGGACAGTCCTGGCCCTGGCTTTG | |
| | | GGCAGGGAGGGAATTTCCCATAGGAAGGGAAGAGTAAAGAGA | |
| | | GAGAGAGAGGTCAGAGTCCAGGTTTGTTGTTCATATGTTTTCTT | |
| | | GAAAGGGCACTATTTCCCAGAATCCAGGTCATCTCTGGGTAGG | |
| | | GAATCCCCTGAATTAGTTTTTTCTGAGAGTATATTAGATTTGCC | |
| | | CTCACAGTAACCCCATGCTGGAAATACCCAACTCCAGTAGCAC | |
| | | TTTTCTCCCTGCCTTTTCCCACTGAAATAACATAATAATATAGG | |
| | | AAGAATACAAGGACTATAGAAATACAGATTAGTGTTTGAACCC | |
| | | TTGCTTACCAGCTACTACTAATATGATTGTGGATGAGGTAGCTT | |
| | | CTTACTTATTAACGGGGATACTAATAGAGGTGGTTCCTTACAAT | |
| | | GATTCCATTTATGATTTTTTATTTAATAGTGATACCAAAGCAAT | |
| | | ACATACTCAGTAGAAACCTACTTCAAGTTCCCATAAAATCATCT | |
| | | GCTTTTCACTTTCAGTACAGTATTTAATAACTTAAATGAGATAT | |
| | | TTCACACTTCAGTAGTAAATACACTTTTTGTTAGATAATTTTGT | |
| | | CCAACTGTATGCTAATGTAAGTGTTCTGAGCATGTTTAAGGCA | |
| | | GGTTAGGTTAAGCTATGATGTTTGGTGGGTTAGGTGTATTAAAT | |
| | | GCATTTCTGATTTTGGATATTTTCAGTGTACAATGGGTTTACAG | |
| | | GGATGTAACCCCATCATAAGTGAAGGAGCACCTGTACTTACTT | |
| | | CATTAAAATGCTGAAACAGTAAATAAGGTAACATTTAATAATA | |
| | | TGTTGTGCAGTTCTTGAAATTTAAGTACTCACCAAATATTACTT | |
| | | TTCCTTTTTTTGTTATTTACTTACTTTTCATTCATTTATTAATTCA | |
| | | TTTGTGCATTTAGTAAACATTTATAAATTATTTCCTGTGCCTGA | |
| | | CAGCATGCTGGAACAGTGCTAAAGATACAAGTTAATTAAGACA | |
| | | CAATCACGACCCCCAAGATTCCTACTCTTTTCTAAAGATTACAG | |
| | | ACAAGCAGACGATGCTATTGTTGAAGAAACATGCTCTGAGAGG | |
| | | CATTTGAAGGAAGTGTAGAGGATAGAAGATGGACACATAACC | |
| | | CAGGATGGGGAGGAAAAGAGTTAGGGAAGGCTTTTTGACGAA | |
| | | GATACTGTTTACACCGTGTGTTCTTATAAATTCATGGTGGTGGG | |
| | | GATAGAGTTGGAGGAAAAGGCATGCTCAGTGGCGTGGAGATG | |
| | | GCAGAGAGATTGGGGTGTTCAAGGATATGCCGGGAATTCAAGG | |
| | | AACGAGAATTCCCATAGACACAGACACAGCTAGACATAGAGA | |
| | | TCTGCAGCTTAGGTTTGGGCTGTGGGTATAGATCCAGGTGGCTT | |
| | | CAACAGACAAAGATCTTTCCTGAGAAAAGGGAAAAGTTTTCAA | |
| | | CACAGAAAGACCATCCCATGTTTGGAATGAGGTTTGCAAATAG | |
| | | ATTGCTTGAGGAGAGAAGTATGTGATCAGAAAGCATTCTTTGT | |
| | | CTATTAACTCCTGCCCAGCAAAAGTGAAAGAAAATTCATGGGA | |
| | | GCATGCAAGAACAAAGAGCACAGCAAAGCTGGACAAACACAG | |
| | | CAATCCAGGCAGGGGATTTCCAACTCAACTCTGGTATATAAGC | |
| | | TGCATGCAAAGTCCTTTTTCTGTCTCTGGTTTCTGGCCCCTTGTC | |
| | | TGCAGAG | |
| SB09409 | UBD1 | TCTCCTTCACCTTCCACCATGAGTAAAAGCTTCTTGAGGCCTCA | 27 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGATTCTAGC | |
| | | CTTACAGCCTAAAAAAGTCAGATTGACACTGGGTAAGAAGCAA | |
| | | GGAGGTCAGGTCCCAAGATGAAGTCCTGCCTGTGGTCAGCAAA | |
| | | GGGGCACCAAGGTGTCTGGGACAGTCCTGGCCCTGGCTTTGGG | |
| | | CAGGGAGGGAATTTCCCATAGGAAGGGAAGAGTAAAGAGAGA | |
| | | GAGAGAGGTCAGAGTCCAGGTTTGTTGTTCATATGTTTTCTTGA | |
| | | AAGGGCACTATTTCCCAGAATCCAGGTCATCTCTGGGTAGGGA | |
| | | ATCCCCTGAATTAGTTTTTTCTGAGAGTATATTAGATTTGCCCT | |
| | | CACAGTAACCCCATGCTGGAAATACCCAACTCCAGTAGCACTT | |
| | | TTCTCCCTGCCTTTTCCCACTGAAATAACATAATAATATAGGAA | |
| | | GAATACAAGGACTATAGAAATACAGATTAGTGTTTGAACCCTT | |
| | | GCTTACCAGCTACTACTAATATGATTGTGGATGAGGTAGCTTCT | |
| | | TACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09410 | UBD1 | TCTCCTTCACCTTCCACCATGAGTAAAAGCTTCTTGAGGCCTCA | 28 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGA | |
| | | CTCTACGGAAGTAGCTTGTTTAAAACCTATAGTCTCTTCGGAGT | |
| | | CGTTCTACTAGTACAAAGGTTTGTTGTTCATATGTTTTCTTGAA | |
| | | AGGGCACTATTTCCCAGAATCCAGGTCATCTCTGGGTAGGGAA | |
| | | TCCCCTGAATTAGTTTTTTCTGAGAGTATATTAGATTTGCCCTC | |
| | | ACAGTAACCCCATGCTGGAAATACCCAACTCCAGTAGCACTTT | |
| | | TCTCCCTGCCTTTTCCCACTGAAATAACATAATAATATAGGAAG | |
| | | AATACAAGGACTATAGAAATACAGATTAGTGTTTGAACCCTTG | |
| | | CTTACCAGCTACTACTAATATGATTGTGGATGAGGTAGCTTCTT | |
| | | ACTTATTAACGGGGATACTAATAGAGGTGGTTCCTTACAATGA | |
| | | TTCCATTTATGATTTTTTATTTAATAGTGATACCAAAGCAATAC | |
| | | ATACTCAGTAGAAACCTACTTCAAGTTCCCATAAAATCATCTGC | |
| | | TTTTCACTTTCAGTACAGTATTTAATAACTTAAATGAGATATTT | |
| | | CACACTTCAGTAGTAAATACACTTTTTGTTAGATAATTTTGTCC | |
| | | AACTGTATGCTAATGTAAGTGTTCTGAGCATGTTTAAGGCAGG | |
| | | TTAGGTTAAGCTATGATGTTTGGTGGGTTAGGTGTATTAAATGC | |
| | | ATTTCTGATTTTGGATATTTTCAGTGTACAATGGGTTTACAGGG | |
| | | ATGTAACCCCATCATAAGTGAAGGAGCACCTGTACTTACTTCA | |
| | | TTAAAATGCTGAAACAGTAAATAAGGTAACATTTAATAATATG | |
| | | TTGTGCAGTTCTTGAAATTTAAGTACTCACCAAATATTACTTTT | |
| | | CCTTTTTTTGTTATTTACTTACTTTTCATTCATTTATTAATTCATT | |
| | | TGTGCATTTAGTAAACATTTATAAATTATTTCCTGTGCCTGACA | |
| | | GCATGCTGGAACAGTGCTAAAGATACAAGTTAATTAAGACACA | |
| | | ATCACGACCCCCAAGATTCCTACTCTTTTCTAAAGATTACAGAC | |
| | | AAGCAGACGATGCTATTGTTGAAGAAACATGCTCTGAGAGGCA | |
| | | TTTGAAGGAAGTGTAGAGGATAGAAGATGGACACATAACCCA | |
| | | GGATGGGGAGGAAAAGAGTTAGGGAAGGCTTTTTGACGAAGA | |
| | | TACTGTTTACACCGTGTGTTCTTATAAATTCATGGTGGTGGGGA | |
| | | TAGAGTTGGAGGAAAAGGCATGCTCAGTGGCGTGGAGATGGC | |
| | | AGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09411 | UBD1 | TCTCCTTCACCTTCCACCATGAGTAAAAGCTTCTTGAGGCCTCA | 29 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTGAGT | |
| | | AAACTAACTTTCAACCGCTCTTCGTGGTCATCTCTGGGTAGGGA | |
| | | ATCCCCTGAATTAGTTTTTTCTGAGAGTATATTAGATTTGCCCT | |
| | | CACAGTAACCCCATGCTGGAAATACCCAACTCCAGTAGCACTT | |
| | | TTCTCCCTGCCTTTTCCCACTGAAATAACATAATAATATAGGAA | |
| | | GAATACAAGGACTATAGAAATACAGATTAGTGTTTGAACCCTT | |
| | | GCTTACCAGCTACTACTAATATGATTGTGGATGAGGTAGCTTCT | |
| | | TACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09412 | UBD1 | TCTCCTTCACCTTCCACCATGAGTAAAAGCTTCTTGAGGCCTCA | 30 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGCTTAAAC | |
| | | ACCGTTTTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09413 | UBD1 | TCTCCTTCACCTTCCACCATGAGTAAAAGCTTCTTGAGGCCTCA | 81 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | GTAATATCATCCGCTCTTTACTGAAATAACATAATAATATAGG | |
| | | AAGAATACAAGGACTATAGAAATACAGATTAGTGTTTGAACCC | |
| | | TTGCTTACCAGCTACTACTAATATGATTGTGGATGAGGTAGCTT | |
| | | CTTACTTATTAACGGGGATACTAATAGAGGTGGTTCCTTACAAT | |
| | | GATTCCATTTATGATTTTTTATTTAATAGTGATACCAAAGCAAT | |
| | | ACATACTCAGTAGAAACCTACTTCAAGTTCCCATAAAATCATCT | |
| | | GCTTTTCACTTTCAGTACAGTATTTAATAACTTAAATGAGATAT | |
| | | TTCACACTTCAGTAGTAAATACACTTTTTGTTAGATAATTTTGT | |
| | | CCAACTGTATGCTAATGTAAGTGTTCTGAGCATGTTTAAGGCA | |
| | | GGTTAGGTTAAGCTATGATGTTTGGTGGGTTAGGTGTATTAAAT | |
| | | GCATTTCTGATTTTGGATATTTTCAGTGTACAATGGGTTTACAG | |
| | | GGATGTAACCCCATCATAAGTGAAGGAGCACCTGTACTTACTT | |
| | | CATTAAAATGCTGAAACAGTAAATAAGGTAACATTTAATAATA | |
| | | TGTTGTGCAGTTCTTGAAATTTAAGTACTCACCAAATATTACTT | |
| | | TTCCTTTTTTTGTTATTTACTTACTTTTCATTCATTTATTAATTCA | |
| | | TTTGTGCATTTAGTAAACATTTATAAATTATTTCCTGTGCCTGA | |
| | | CAGCATGCTGGAACAGTGCTAAAGATACAAGTTAATTAAGACA | |
| | | CAATCACGACCCCCAAGATTCCTACTCTTTTCTAAAGATTACAG | |
| | | ACAAGCAGACGATGCTATTGTTGAAGAAACATGCTCTGAGAGG | |
| | | CATTTGAAGGAAGTGTAGAGGATAGAAGATGGACACATAACC | |
| | | CAGGATGGGGAGGAAAAGAGTTAGGGAAGGCTTTTTGACGAA | |
| | | GATACTGTTTACACCGTGTGTTCTTATAAATTCATGGTGGTGGG | |
| | | GATAGAGTTGGAGGAAAAGGCATGCTCAGTGGCGTGGAGATG | |
| | | GCAGAGAGATTGGGGTGTTCAAGGATATGCCGGGAATTCAAGG | |
| | | AACGAGAATTCCCATAGACACAGACACAGCTAGACATAGAGA | |
| | | TCTGCAGCTTAGGTTTGGGCTGTGGGTATAGATCCAGGTGGCTT | |
| | | CAACAGACAAAGATCTTTCCTGAGAAAAGGGAAAAGTTTTCAA | |
| | | CACAGAAAGACCATCCCATGTTTGGAATGAGGTTTGCAAATAG | |
| | | ATTGCTTGAGGAGAGAAGTATGTGATCAGAAAGCATTCTTTGT | |
| | | CTATTAACTCCTGCCCAGCAAAAGTGAAAGAAAATTCATGGGA | |
| | | GCATGCAAGAACAAAGAGCACAGCAAAGCTGGACAAACACAG | |
| | | CAATCCAGGCAGGGGATTTCCAACTCAACTCTGGTATATAAGC | |
| | | TGCATGCAAAGTCCTTTTTCTGTCTCTGGTTTCTGGCCCCTTGTC | |
| | | TGCAGAG | |
| SB09414 | UBD1 | TCTCCTTCACCTTCCACCATGAGTAAAAGCTTCTTGAGGCCTCA | 82 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTGATCGG | |
| | | CCAATATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09415 | UBD1 | TCTCCTTCACCTTCCACCATGAGTAAAAGCTTCTTGAGGCCTCA | 88 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTTAGAACTTCGTATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09416 | UBD1 | TCTCCTTCACCTTCCACCATGAGTAAAAGCTTCTTGAGGCCTCA | 89 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTAACATTAAGTAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09417 | UBD1 | TCTCCTTCACCTTCCACCATGAGTAAAAGCTTCTTGAGGCCTCA | 90 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTATAGATAACGCC | |
| | | GTCATTGTAAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09418 | UBD1 | TCTCCTTCACCTTCCACCATGAGTAAAAGCTTCTTGAGGCCTCA | 91 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACTTTCTCTAACGGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09419 | UBD1 | TCTCCTTCACCTTCCACCATGAGTAAAAGCTTCTTGAGGCCTCA | 92 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCACTAACATCGTTCTCAGCTAAAGATTACAG | |
| | | ACAAGCAGACGATGCTATTGTTGAAGAAACATGCTCTGAGAGG | |
| | | CATTTGAAGGAAGTGTAGAGGATAGAAGATGGACACATAACC | |
| | | CAGGATGGGGAGGAAAAGAGTTAGGGAAGGCTTTTTGACGAA | |
| | | GATACTGTTTACACCGTGTGTTCTTATAAATTCATGGTGGTGGG | |
| | | GATAGAGTTGGAGGAAAAGGCATGCTCAGTGGCGTGGAGATG | |
| | | GCAGAGAGATTGGGGTGTTCAAGGATATGCCGGGAATTCAAGG | |
| | | AACGAGAATTCCCATAGACACAGACACAGCTAGACATAGAGA | |
| | | TCTGCAGCTTAGGTTTGGGCTGTGGGTATAGATCCAGGTGGCTT | |
| | | CAACAGACAAAGATCTTTCCTGAGAAAAGGGAAAAGTTTTCAA | |
| | | CACAGAAAGACCATCCCATGTTTGGAATGAGGTTTGCAAATAG | |
| | | ATTGCTTGAGGAGAGAAGTATGTGATCAGAAAGCATTCTTTGT | |
| | | CTATTAACTCCTGCCCAGCAAAAGTGAAAGAAAATTCATGGGA | |
| | | GCATGCAAGAACAAAGAGCACAGCAAAGCTGGACAAACACAG | |
| | | CAATCCAGGCAGGGGATTTCCAACTCAACTCTGGTATATAAGC | |
| | | TGCATGCAAAGTCCTTTTTCTGTCTCTGGTTTCTGGCCCCTTGTC | |
| | | TGCAGAG | |
| SB09420 | UBD1 | TCTCCTTCACCTTCCACCATGAGTAAAAGCTTCTTGAGGCCTCA | 96 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCT | |
| | | ATACAGTGTTCAGCGTGTTACTTGTGAGGCTTTTTGACGAAGAT | |
| | | ACTGTTTACACCGTGTGTTCTTATAAATTCATGGTGGTGGGGAT | |
| | | AGAGTTGGAGGAAAAGGCATGCTCAGTGGCGTGGAGATGGCA | |
| | | GAGAGATTGGGGTGTTCAAGGATATGCCGGGAATTCAAGGAAC | |
| | | GAGAATTCCCATAGACACAGACACAGCTAGACATAGAGATCTG | |
| | | CAGCTTAGGTTTGGGCTGTGGGTATAGATCCAGGTGGCTTCAA | |
| | | CAGACAAAGATCTTTCCTGAGAAAAGGGAAAAGTTTTCAACAC | |
| | | AGAAAGACCATCCCATGTTTGGAATGAGGTTTGCAAATAGATT | |
| | | GCTTGAGGAGAGAAGTATGTGATCAGAAAGCATTCTTTGTCTA | |
| | | TTAACTCCTGCCCAGCAAAAGTGAAAGAAAATTCATGGGAGCA | |
| | | TGCAAGAACAAAGAGCACAGCAAAGCTGGACAAACACAGCAA | |
| | | TCCAGGCAGGGGATTTCCAACTCAACTCTGGTATATAAGCTGC | |
| | | ATGCAAAGTCCTTTTTCTGTCTCTGGTTTCTGGCCCCTTGTCTGC | |
| | | AGAG | |
| SB09421 | UBD1 | TCTCCTTCACCTTCCACCATGAGTAAAAGCTTCTTGAGGCCTCA | 97 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTCGTACAAGTATCATGCTCAGTGGCGTGGAGATGGC | |
| | | AGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09422 | UBD1 | TCTCCTTCACCTTCCACCATGAGTAAAAGCTTCTTGAGGCCTCA | 119 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCAGTCTCTGAATAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAGGCAGGGGATTTCCAACTCAACTCTGGTATATAAGCT | |
| | | GCATGCAAAGTCCTTTTTCTGTCTCTGGTTTCTGGCCCCTTGTCT | |
| | | GCAGAG | |
| SB09423 | UBD1 | TCTCCTTCACCTTCCACCATGAGTAAAAGCTTCTTGAGGCCTCA | 120 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCTATATAATACCCGCTAGCATACAAATAGC | |
| | | ATGCAAGAACAAAGAGCACAGCAAAGCTGGACAAACACAGCA | |
| | | ATCCAGGCAGGGGATTTCCAACTCAACTCTGGTATATAAGCTG | |
| | | CATGCAAAGTCCTTTTTCTGTCTCTGGTTTCTGGCCCCTTGTCTG | |
| | | CAGAG | |
| SB09424 | UBD1 | TCTCCTTCACCTTCCACCATGAGTAAAAGCTTCTTGAGGCCTCA | 121 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCGTTGCTCATATACACAGCAAAGCTGGACAAACACAGCA | |
| | | ATCCAGGCAGGGGATTTCCAACTCAACTCTGGTATATAAGCTG | |
| | | CATGCAAAGTCCTTTTTCTGTCTCTGGTTTCTGGCCCCTTGTCTG | |
| | | CAGAG | |
| SB09425 | UBD1 | TCTCCTTCACCTTCCACCATGAGTAAAAGCTTCTTGAGGCCTCA | 122 |
| | ablation | CCAGAAGCAGATGCTGGTGCCATACTTCTTGTATAGTTGGAGG | |
| | variant | AACTATGAATCAATTAAACTTTTTTTCCTTATAAATTACCCAGC | |
| | | CTCAAGTATTCCTTTATAGTATGCAAAAGAGACTAAAACAGCA | |
| | | AATGAAAAGAGATCTGTCTCTGAAAGAACTTATTGTGACATAG | |
| | | AGAGAGACAGAAAAAAGTCAGATTGACACTGGGTAAGAAGCA | |
| | | AGGAGGTCAGGTCCCAAGATGAAGTCCTGCCTGTGGTCAGCAA | |
| | | AGGGGCACCAAGGTGTCTGGGACAGTCCTGGCCCTGGCTTTGG | |
| | | GCAGGGAGGGAATTTCCCATAGGAAGGGAAGAGTAAAGAGAG | |
| | | AGAGAGAGGTCAGAGTCCAGGTTTGTTGTTCATATGTTTTCTTG | |
| | | AAAGGGCACTATTTCCCAGAATCCAGGTCATCTCTGGGTAGGG | |
| | | AATCCCCTGAATTAGTTTTTTCTGAGAGTATATTAGATTTGCCC | |
| | | TCACAGTAACCCCATGCTGGAAATACCCAACTCCAGTAGCACT | |
| | | TTTCTCCCTGCCTTTTCCCACTGAAATAACATAATAATATAGGA | |
| | | AGAATACAAGGACTATAGAAATACAGATTAGTGTTTGAACCCT | |
| | | TGCTTACCAGCTACTACTAATATGATTGTGGATGAGGTAGCTTC | |
| | | TTACTTATTAACGGGGATACTAATAGAGGTGGTTCCTTACAATG | |
| | | ATTCCATTTATGATTTTTTATTTAATAGTGATACCAAAGCAATA | |
| | | CATACTCAGTAGAAACCTACTTCAAGTTCCCATAAAATCATCTG | |
| | | CTTTTCACTTTCAGTACAGTATTTAATAACTTAAATGAGATATT | |
| | | TCACACTTCAGTAGTAAATACACTTTTTGTTAGATAATTTTGTC | |
| | | CAACTGTATGCTAATGTAAGTGTTCTGAGCATGTTTAAGGCAG | |
| | | GTTAGGTTAAGCTATGATGTTTGGTGGGTTAGGTGTATTAAATG | |
| | | CATTTCTGATTTTGGATATTTTCAGTGTACAATGGGTTTACAGG | |
| | | GATGTAACCCCATCATAAGTGAAGGAGCACCTGTACTTACTTC | |
| | | ATTAAAATGCTGAAACAGTAAATAAGGTAACATTTAATAATAT | |
| | | GTTGTGCAGTTCTTGAAATTTAAGTACTCACCAAATATTACTTT | |
| | | TCCTTTTTTTGTTATTTACTTACTTTTCATTCATTTATTAATTCAT | |
| | | TTGTGCATTTAGTAAACATTTATAAATTATTTCCTGTGCCTGAC | |
| | | AGCATGCTGGAACAGTGCTAAAGATACAAGTTAATTAAGACAC | |
| | | AATCACGACCCCCAAGATTCCTACTCTTTTCTAAAGATTACAGA | |
| | | CAAGCAGACGATGCTATTGTTGAAGAAACATGCTCTGAGAGGC | |
| | | ATTTGAAGGAAGTGTAGAGGATAGAAGATGGACACATAACCC | |
| | | AGGATGGGGAGGAAAAGAGTTAGGGAAGGCTTTTTGACGAAG | |
| | | ATACTGTTTACACCGTGTGTTCTTATAAATTCATGGTGGTGGGG | |
| | | ATAGAGTTGGAGGAAAAGGCATGCTCAGTGGCGTGGAGATGG | |
| | | CAGAGAGATTGGGGTGTTCAAGGATATGCCGGGAATTCAAGGA | |
| | | ACGAGAATTCCCATAGACACAGACACAGCTAGACATAGAGATC | |
| | | TGCAGCTTAGGTTTGGGCTGTGGGTATAGATCCAGGTGGCTTC | |
| | | AACAGACAAAGATCTTTCCTGAGAAAAGGGAAAAGTTTTCAAC | |
| | | ACAGAAAGACCATCCCATGTTTGGAATGAGGTTTGCAAATAGA | |
| | | TTGCTTGAGGAGAGAAGTATGTGATCAGAAAGCATTCTTTGTC | |
| | | TATTAACTCCTGCCCAGCAAAAGTGAAAGAAAATTCATGGGAG | |
| | | CATGCAAGAACAAAGAGCACAGCAAAGCTGGACAAACACAGC | |
| | | AATCCAACGTCTGTTAGTAGTACTCAACTCTGGTATATAAGCTG | |
| | | CATGCAAAGTCCTTTTTCTGTCTCTGGTTTCTGGCCCCTTGTCTG | |
| | | CAGAG | |
| SB08123 | Synthetic | GATGAATTAATGAACAGATACCTGAATAAAAGGATAGAAGGC | 123 |
| | variant of | CAGGCACGGTGGCTCACGCCTGTAATCCCAACACTTTGGGAAG | |
| | CCL19 | CGGAGGCAAGCAGATCATTTGAAGTCAGGAGTTCGAAACCAG | |
| | promoter | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAGGTGAATTTTCTAACAGAGC | |
| | | TTATTGTGCGTCAGCCTCTAAGCTAAGGGCTTTACTCAGGGTTC | |
| | | CTCACTTAATCCTAAGAAGTAGATCTTCTACCATTCCCATTTTA | |
| | | CAAATAAGGACACTAAGACACAGAAACTGGAGAATTTTCACCT | |
| | | TGTCGCAGTTAGTAAGTGGTGGAATCTGAATCCCAAGCTAGGC | |
| | | GCTCTGTCTGCAGAGTCTCCCTCCTCTGCACCGTGACTACCCGT | |
| | | GCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTCACATTTAGTTTCTCTTTCAAGGCCTTCTCTT | |
| | | CTGACCTTTCTCATAAGGATGGGAAGTCACTTGTCCCCTCCCCC | |
| | | ATCGGCCGATTCCTTGAAATTTTAAGATTTGTCTGAATATGAGC | |
| | | TGGCCAGCCCCTCTCCTTAGAAATCCCCTCTTGGTTCCCAGGAC | |
| | | TGCGCGTAGAACTTCGTAACATTAAGCTATTCCAAGCCAGTCTC | |
| | | CAGATAACGCCGTCATTGTATCAGGTGTTGGACATAGGAAAAC | |
| | | AGTTAACATCGTTCTCAGCTAGTTTGCTGAGCCCCAGAGGCCTG | |
| | | AGATGTGGGATGGGGGTCCTGGGCCTCCTCCCAAGGTACCTGG | |
| | | CCTTTCAGCTCTGCTTGCCCCAAGGGAAGGACTGGTTCCCGAC | |
| | | GTCTGTTAGTAGTATTACCCGTGTATTTCGGTCTTCGAGCAATT | |
| | | ACTTTATTGTCCTGAGGGTGCCAGGGAGCCTTCTTCACCACCAG | |
| | | CCCAAGAAAGAAGCACCAGTGAGGACAAGGGATAAACCTAAG | |
| | | GAAGGGAGCACAGTTCCTCACTGTGGGCCCCACCTTTGACCAT | |
| | | CACTTCTGAACCCCAGCTTCATGGTTGATGATGCAAATGCCATC | |
| | | AATGGGATGACAGGGCAATCCCTCTCTCTGGAGAAACAGTGCA | |
| | | CAGCCCCCTTTACTGGGCCAGACTGCAGACTTAAGTTCTCAGA | |
| | | GCAGGAGCCAAGGAAAGCAGAAGGAAAGGGCAACCGCAGGG | |
| | | GGAAGAAGGGGGGCAGGGTGCGATTCTGCCAGAAAGAGGGGG | |
| | | GCAACAGGGGAATCACAGAATGGGACATGAAGGGGAATTTCA | |
| | | GGCAGAGAAAGTGAAGCTCTGGCCATAAATAGGAGTCAGCTG | |
| | | CAGGGCTCACATTCCCAGCCTCACATCACTCACACCTTGCATTT | |
| | | CACCCCTGCATCCCAGTCGCCCTGCAGCCTCACACAGATCCTGC | |
| | | ACACACCCAGACAGCTGGCGCTCACACATTCACCGTTGGCCTG | |
| | | CCTCTGTTCACCCTCC | |
| SB08124 | Synthetic | GATGAATTAATGAACAGATACCTGAATAAAAGGATAGAAGGC | 124 |
| | variant of | CAGGCACGGTGGCTCACGCCTGTAATCCCAACACTTTGGGAAG | |
| | CCL19 | CGGAGGCAAGCAGATCATTTGAAGTCAGGAGTTCGAAACCAG | |
| | promoter | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGAGGTGAATTTTCTAACAGAGC | |
| | | TTATTGTGCGTCAGCCTCTAAGCTAAGGGCTTTACTCAGGGTTC | |
| | | CTCACTTAATCCTAAGAAGTAGATCTTCTACCATTCCCATTTTA | |
| | | CAAATAAGGACACTAAGACACAGAAACTGGAGAATTTTCACCT | |
| | | TGTCGCAGTTAGTAAGTGGTGGAATCTGAATCCCAAGCTAGGC | |
| | | GCTCTGTCTGCAGAGTCTCCCTCCTCTGCACCGTGACTACCCGT | |
| | | GCTGTGCCATGACCAAGTGGCAGACAGGCGGTGTGGAGTGGA | |
| | | GGTAAAGACTGCAAACCCTAGAGCACACCTGGGTCCAAATCAT | |
| | | GCTCCAGTACTTACTAGCTGTGTGGCTTTGTGCTCTGAGCCTCA | |
| | | TGTTCCTTATGTGTAAAATAAGAATAATAATGTGAAAATAAGA | |
| | | AAAACTACATCAATGGTATTGTTTTGGGAAAGTTAAGCAAGGT | |
| | | AATAGTGGTAAAGTCCTTGACATAGAGCCTAGCACATAGTAAA | |
| | | CAATCAAAAATACCATTACTATTATTATGATTAATGAATGGAA | |
| | | AGAAGCTAAGTCAGAAATTTAAGAGGGAAATGTTCCCCCTCCC | |
| | | TCTCTCCATCCAAAAGAGGGTCTAGGGTTTGGGTGACGACGGG | |
| | | TGACTGGGCGTTTGAGTAAACTAACTTTCAACCGCTCTTTCTCT | |
| | | TCTGACCTTTCTCATAAGGATGGGAAGTCACTTGTCCCCTCCCC | |
| | | CATCGGCCGATTCCTTGAAATTTTAAGATTTGTCTGAATATGAG | |
| | | CTGGCCAGCCCCTCTCCTTAGAAATCCCCTCTTGGTTCCCAGGA | |
| | | CTGCGCGTAGAACTTCGTAACATTAAGCTATTCCAAGCCAGTCT | |
| | | CCAGATAACGCCGTCATTGTATCAGGTGTTGGACATAGGAAAA | |
| | | CAGTTAACATCGTTCTCAGCTAGTTTGCTGAGCCCCAGAGGCCT | |
| | | GAGATGTGGGATGATATACAGTGTTCAGCGTGTTACGTACCTG | |
| | | GCCTTTCAGCTCTGCTTGCCCCAAGGGAAGGACTGGTTCCCGA | |
| | | CGTCTGTTAGTAGTATTACCCGTGTATTTCGGTCTTCGAGCAAT | |
| | | TACTTTATTGTCCTGAGGGTGCCAGGGAGCCTTCTTCACCACCA | |
| | | GCCCAAGAAAGAAGCACCAGTGAGGACAAGGGATAAACCTAA | |
| | | GGAAGGGAGCACAGTTCCTCACTGTGGGCCCCACCTTTGACCA | |
| | | TCACTTCTGAACCCCAGCTTCATGGTTGATGATGCAAATGCCAT | |
| | | CAATGGGATGACAGGGCAATCCCTCTCTCTGGAGAAACAGTGC | |
| | | ACAGCCCCCTTTACTGGGCCAGACTGCAGACTTAAGTTCTCAG | |
| | | AGCAGGAGCCAAGGAAAGCAGAAGGAAAGGGCAACCGCAGG | |
| | | GGGAAGAAGGGGGGCAGGGTGCGATTCTGCCAGAAAGAGGGG | |
| | | GGCAACAGGGGAATCACAGAATGGGACATGAAGGGGAATTTC | |
| | | AGGCAGAGAAAGTGAAGCTCTGGCCATAAATAGGAGTCAGCT | |
| | | GCAGGGCTCACATTCCCAGCCTCACATCACTCACACCTTGCATT | |
| | | TCACCCCTGCATCGCAGAATATCGATATCTTCACACAGATCCTG | |
| | | CACACACCCAGACAGCTGGCGCTCACACATTCACCGTTGGCCT | |
| | | GCCTCTGTTCACCCTCC | |
| SB08125 | Synthetic | GATGAATTAATGAACAGATACCTGAATAAAAGGATAGAAGGC | 125 |
| | variant of | CAGGCACGGTGGCTCACGCCTGTAATCCCAACACTTTGGGAAG | |
| | CCL19 | CGGAGGCAAGCAGATCATTTGAAGTCAGGAGTTCGAAACCAG | |
| | promoter | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGATAACAGAGCTTATTGTGCGT | |
| | | CAGCCTCTAAGCTAAGGGCTTTACTCAGGGTTCCTCACTTAATC | |
| | | CTAAGAAGTAGATCTTCTACCATTCCCATTTTACAAATAAGGAC | |
| | | ACTAAGACACAGAAACTGGAGAATTTTCACCTTGTCGCAGTTA | |
| | | GTAAGTGGTGGAATCTGAATCCCAAGCTAGGCGCTCTGTCTGC | |
| | | AGAGTCTCCCTCCTCTGCACCGTGACTACCCGTGCTGTGCCATG | |
| | | ACCAAGTGGCAGACAGGCGGTGTGGAGTGGAGGTAAAGACTG | |
| | | CAAACCCTAGAGCACACCTGGGTCCAAATCATGCTCCAGTACT | |
| | | TACTAGCTGTGTGGCTTTGTGCTCTGAGCCTCATGTTCCTTATG | |
| | | TGTAAAATAAGAATAATAATGTGAAAATAAGAAAAACTACATC | |
| | | AATGGTATTGTTTTGGGAAAGTTAAGCAAGGTAATAGTGGTAA | |
| | | AGTCCTTGACATAGAGCCTAGCACATAGTAAACAATCAAAAAT | |
| | | ACCATTACTATTATTATGATTAATGAATGGAAAGAAGCTAAGT | |
| | | CAGAAATTTAAGAGGGAAATGTTCCCCCTCCCTCTCTCCATCCA | |
| | | AAAGAGGGTCTAGGGTTTGGGTGACGACGGGTGACTGGGCGTT | |
| | | TCACATTTAGTTTCTCTTTCAAGGCCTTCTCTTCTGACCTTTCTC | |
| | | ATAAGGATGGGAAGTCACTTGTCCCCTCCCCCATCGGCCGATT | |
| | | CCTTGAAATTTTAAGATTTGTCTGAATATGAGCTGGCCAGCCCC | |
| | | TCTCCTTAGAAATCCCCTCTTGGTTCCCAGGACTGGCTATTCCA | |
| | | AGCCAGTCTCCCAGGTGTTGGACATAGGAAAACAGTGTTTGCT | |
| | | GAGCCCCAGAGGCCTGAGATGTGGGATGGGGGTCCTGGGCCTC | |
| | | CTCCCAAGGTACCTGGCCTTTCAGCTCTGCTTGCCCCAAGGGAA | |
| | | GGACTGGTTCCCTTGTCCTGAGGGTGCCAGGGAGCCTTCTTCAC | |
| | | CACCAGCCCAAGAAAGAAGCACCAGTGAGGACAAGGGATAAA | |
| | | CCTAAGGAAGGGAGCACAGTTCCTCACTGTGGGCCCCACCTTT | |
| | | GACCATCACTTCTGAACCCCAGCTTCATGGTTGATGATGCAAAT | |
| | | GCCATCAATGGGATGACAGGGCAATCCCTCTCTCTGGAGAAAC | |
| | | AGTGCACAGCCCCCTTTACTGGGCCAGACTGCAGACTTAAGTT | |
| | | CTCAGAGCAGGAGCCAAGGAAAGCAGAAGGAAAGGGCAACCG | |
| | | CAGGGGGAAGAAGGGGGGCAGGGTGCGATTCTGCCAGAAAGA | |
| | | GGGGGGCAACAGGGGAATCACAGAATGGGACATGAAGGGGAA | |
| | | TTTCAGGCAGAGAAAGTGAAGCTCTGGCCATAAATAGGAGTCA | |
| | | GCTGCAGGGCTCACATTCCCAGCCTCACATCACTCACACCTTGC | |
| | | ATTTCACCCCTGCATCCCAGTCGCCCTGCAGCCTCACACAGATC | |
| | | CTGCACACACCCAGACAGCTGGCGCTCACACATTCACCGTTGG | |
| | | CCTGCCTCTGTTCACCCTCC | |
| SB08126 | Synthetic | GATGAATTAATGAACAGATACCTGAATAAAAGGATAGAAGGC | 126 |
| | variant of | CAGGCACGGTGGCTCACGCCTGTAATCCCAACACTTTGGGAAG | |
| | CCL19 | CGGAGGCAAGCAGATCATTTGAAGTCAGGAGTTCGAAACCAG | |
| | promoter | GAGTTCGAAACCAGCCTAGCCAACAAGGTGAAACCTCACCTCT | |
| | | ACTAAAAATACCAAATTTAGCCGGGCATGATGGTGGGTGCCTG | |
| | | TAATCCCAGCTACTCAGGAGGCTGAGGCAGGAGAATCCCTTGA | |
| | | GCCCAGGAGGTGGAGGTTGCTCTGAGCCAAGATCGTGCCATTG | |
| | | TACTCCAGCCTGGGTGACAGAGTGAGACTCTGTCTCAAAAAAA | |
| | | AAAAAAAAAAAAGGACAAAAGATAACAGAGCTTATTGTGCGT | |
| | | CAGCCTCTAAGCTAAGGGCTTTACTCAGGGTTCCTCACTTAATC | |
| | | CTAAGAAGTAGATCTTCTACCATTCCCATTTTACAAATAAGGAC | |
| | | ACTAAGACACAGAAACTGGAGAATTTTCACCTTGTCGCAGTTA | |
| | | GTAAGTGGTGGAATCTGAATCCCAAGCTAGGCGCTCTGTCTGC | |
| | | AGAGTCTCCCTCCTCTGCACCGTGACTACCCGTGCTGTGCCATG | |
| | | ACCAAGTGGCAGACAGGCGGTGTGGAGTGGAGGTAAAGACTG | |
| | | CAAACCCTAGAGCACACCTGGGTCCAAATCATGCTCCAGTACT | |
| | | TACTAGCTGTGTGGCTTTGTGCTCTGAGCCTCATGTTCCTTATG | |
| | | TGTAAAATAAGAATAATAATGTGAAAATAAGAAAAACTACATC | |
| | | AATGGTATTGTTTTGGGAAAGTTAAGCAAGGTAATAGTGGTAA | |
| | | AGTCCTTGACATAGAGCCTAGCACATAGTAAACAATCAAAAAT | |
| | | ACCATTACTATTATTATGATTAATGAATGGAAAGAAGCTAAGT | |
| | | CAGAAATTTAAGAGGGAAATGTTCCCCCTCCCTCTCTCCATCCA | |
| | | AAAGAGGGTCTAGGGTTTGGGTGACGACGGGTGACTGGGCGTT | |
| | | TTCTCTTCTGACCTTTCTCATAAGGATGGGAAGTCACTTGTCCC | |
| | | CTCCCCCATCGGCCGATTCCTTGAAATTTTAAGATTTGTCTGAA | |
| | | TATGAGCTGGCCAGCCCCTCTCCTTAGAAATCCCCTCTTGGTTC | |
| | | CCAGGACTGGCTATTCCAAGCCAGTCTCCCAGGTGTTGGACAT | |
| | | AGGAAAACAGTGTTTGCTGAGCCCCAGAGGCCTGAGATGTGGG | |
| | | ATGGTACCTGGCCTTTCAGCTCTGCTTGCCCCAAGGGAAGGAC | |
| | | TGGTTCCCTTGTCCTGAGGGTGCCAGGGAGCCTTCTTCACCACC | |
| | | AGCCCAAGAAAGAAGCACCAGTGAGGACAAGGGATAAACCTA | |
| | | AGGAAGGGAGCACAGTTCCTCACTGTGGGCCCCACCTTTGACC | |
| | | ATCACTTCTGAACCCCAGCTTCATGGTTGATGATGCAAATGCCA | |
| | | TCAATGGGATGACAGGGCAATCCCTCTCTCTGGAGAAACAGTG | |
| | | CACAGCCCCCTTTACTGGGCCAGACTGCAGACTTAAGTTCTCA | |
| | | GAGCAGGAGCCAAGGAAAGCAGAAGGAAAGGGCAACCGCAG | |
| | | GGGGAAGAAGGGGGGCAGGGTGCGATTCTGCCAGAAAGAGGG | |
| | | GGGCAACAGGGGAATCACAGAATGGGACATGAAGGGGAATTT | |
| | | CAGGCAGAGAAAGTGAAGCTCTGGCCATAAATAGGAGTCAGC | |
| | | TGCAGGGCTCACATTCCCAGCCTCACATCACTCACACCTTGCAT | |
| | | TTCACCCCTGCATTCACACAGATCCTGCACACACCCAGACAGC | |
| | | TGGCGCTCACACATTCACCGTTGGCCTGCCTCTGTTCACCCTCC | |
| SB09758 | LNCARO | AAGGTCTCTCTCTGTTACCCAGGCTGGAGTGCAATGGAATATG | 414 |
| | Dv1 | CATAGCTTACTACAGCCTCTATTCTTGGGCTCAAGCAATCCACC | |
| | | TACCTCAGCTTCCCAAGTAGATAGGACTACACGTGCACCACCA | |
| | | TGCTCTGCTAATTAAAAAAACAAAATCTGTAGAGATGGAGCTT | |
| | | TGCTGTGTTACCCAATCTGGTCTCAAATTCCTGGCCTCAAGTAG | |
| | | TCCTCCTGCCTCAGCCTCACAAAGCACTAGGATTACAGGCGTG | |
| | | AGCCATTGTGCCTGGCCTTACTGTAGAACTTTATAATTCATTAT | |
| | | CTTCTTTACCCAGGTGTACATAGTATTAGTGAAATAAAATACAT | |
| | | TAAATTAAAGCCATTTTTAATTTTTTGGTATACTATGGAATTAG | |
| | | GCATATTTGAAATTACTTCTCATATGACATTGTAAACATAGCCA | |
| | | AATTATTATCATTTTCTTCCTGTAGCAATAAGTCTGAACATAAA | |
| | | ATCTTCCATTTTCTTATACAAAATGAAGCTTTAAAAAATCAAGA | |
| | | GCATTTAATTTGTTTCATATATAGGCCTTCTCAACTGCGATCTT | |
| | | GCCCCAGAGAAGAGAAAAAACTACACAGAAACCTTAAGCATT | |
| | | AAGACACATTATTGTAACTAAGAAAGAGCATAACGTTAATAGT | |
| | | GGCTTTTTCTAAGGTGTAAAATAATGAATTTTTAAGATACTTTT | |
| | | CCTTATTTTTCATATTTTGTATATTAAATATATACCACTTCTATC | |
| | | ATTTAAAATATCTATTATGTTTTTAAAATAAAGGCTTTCTTTCC | |
| | | GCTTTCTTTCAGGCCAGAGGTAAAAGAAACAGAGAAGAAGAG | |
| | | ATAATAATTCTAACAGAAATATTACAGCCTTTACAACTTGCATG | |
| | | CATACAGTTATACATTAAGAGACTGAAAAAGGCAGTTTCTTTT | |
| | | ACTGCTAATTCTAGTAGTGATGAAACTCAGGCTCAAGTATTCA | |
| | | GGTGACTTGTTTGAGGTCACACAGTGCATAAATGGTTGCACCT | |
| | | GAGCTGAAATATGAGTTGTCCAACTCTTGTAAGTGCATGCTCC | |
| | | ATCACATCATCCTGAGTCAGTAATGAGGATGCCATTGTACCCTT | |
| | | CAAGCAGCCCTGGAGAGTTTATCTCTGCATGCTGGAGCTAATA | |
| | | ATAATAGTGAGTATTTATATAGTCCACACTAGGTACCACATTCT | |
| | | GCTTTAAGTGCTTTACAAATAATAACTCATTTCATCCATACAGC | |
| | | AACTTCATGGGGTCGATACCATTACTATCATCCTGTTTTACATA | |
| | | TAAGGAAACTGAGGTTACATAACTAGGCCCTAGATCACTCAGC | |
| | | TAGAGGTAAGACTGGGATACAAGCAGACTGACTTCAGAGCCCA | |
| | | TGCTTCACCATCACATTAAGTAGCCTGCTGATTTGTCTTTATTT | |
| | | GTTATGTTAACAGGAAAGGATTGCTTTCCTTTAGTGCAAGGTA | |
| | | AGCCACAAGGCAGATTATAGGAAATAGTCTACAAAAACTGAA | |
| | | GGGAGCAGCATCAATTTTCCCAAGGAATTTTTTTTCCTTTATAC | |
| | | TTCTTTATCATTAGAGCCCAATTTGGACAAATTCTTTTCCACTCT | |
| | | GAGCCTCAGTCAATGTTTGGCTCATTTCATGAAGGAAACTGAT | |
| | | GGTAGAGGCAAGAGTACACTGCTCAGCAGACCCTTTGTCCAAG | |
| | | GGTAATTTCATTGGCCTCCACCATTGTTTGCCTGTTAAACTGTG | |
| | | GCTTACAACATTTCAAGGAGCAAAGACAAAATGTTACACTTGT | |
| | | ACTGTGGTGAAACTTTCCATTTCAAAGAAATAGATTCCAGCAG | |
| | | GCGTAAAGCTACCTGGAAGCAAAGGGTTTGTTATGGGGTTATT | |
| | | ATAAAATAGAAAAACAAAAACTCAATCTGGAACATGACCCCTT | |
| | | TGTCGTACACATGCATTGCATCTGGCCTGCTGTACAATGTGGAC | |
| | | CACTGGCAGAGACTGTGTTCACACATGGAACACAGGAGAGAG | |
| | | CTACTCTAAGAAGAAAAAGTTAGGAAAACTAGATTCTTTTCTTT | |
| | | T | |
| SB09760 | LNCARO | GGAGAAGAAGGAGTCATAGAGAAAGGCTTTTCATCAGTCATGG | 415 |
| | Dv3 | ACCTAAAAGCGTGCTCTGTAATGGTGGACTTCAGACAGAGATA | |
| | | GAGAGATCCTAGATGAGTCATCCTGGGATGCAGGATGCAGTTG | |
| | | CTTCTTTTGTTTTGAGAATGCTTGGCTGCTTGCTCCATTCCTACT | |
| | | CATCAGTAGAAGCTTGTCTTAGTGTTTCATTTTCAAGGACCTCT | |
| | | TGATATAAACTACGGTTTGAAGGACATTCCGTTCCTAAGTGGG | |
| | | ACAGGAGCAGAGCCTGGGCTGCCATGACCAGTTCTGACATATC | |
| | | TCAGGTTGCTGCATTACCAGAGCATTCCAAAGTTATTCCTGAGA | |
| | | ACTACAAGTGAGAAAGGTTGGAAAACTGGGTCAGTTCAAGGCC | |
| | | TCCCAAAGAACCATCCTGCAGCTATCACGAGATATGTCTGTCT | |
| | | GTCTGTCTCTTAAGTAGAGAAGGCAGAGATTAAAAATAAAAAA | |
| | | GATTGGAACAACAAAGATATAGGGAAAAATAATTTTGGAAGCT | |
| | | ACCTTTGTGAAATCTGTAGAAAGATGTAGAGAAGAGGAGCTGG | |
| | | TAACAGCCATGTCTGGACTCCCGGTCACCCTATTACCACAACCT | |
| | | GTTACCCCAACCCCATCACTTCACATAAGAAGCAAAGTTCATG | |
| | | CTAGCCTAGTAGCCCATTTCCTCCAAGACAATTCCCATCAAGCC | |
| | | TGGCCAGGTGGCTCAGCCCAGACCGAAGCATGGAGGATGTGCA | |
| | | GTGTGAAAAAGCACAGACTGAGATGACCATGCAGAAAAGATT | |
| | | TGTATAAGAAAGCTGGGGCCACCCCTGTAGCCTTGTTTTTATAA | |
| | | TACTTTCCTGTGTCAAAAAAATTAATGAAGTTAATTTAGAATGA | |
| | | GAAATGATACATAATGCCTATAGGTATCTCTGAGAAGATTGCT | |
| | | ACAGAGACTAAGAGGATCATAGACCTTCCTTCTGGAATGTTCC | |
| | | TGTTCCTATCTGTAGGATGAATGAGTGGAATCAGTGACTCAGA | |
| | | CAAAGCCACTGTTGCACAACTGTATGCATGCACAGTGAACTCT | |
| | | GCAGACTCTGCAGATGGTTCCTGTAAATAGCGCTTAAAACCAC | |
| | | AGTGGAAAAGCCACATGCTCCTGATACACTTGGGGGGACATAA | |
| | | AGGGAAACCCACACACCCAAGGAGGAGGGCTCTGGCCATGCA | |
| | | AGGCTGAAGGCCCAGTGTACTGATATGAAAACTGAAGCTCAGA | |
| | | CAGAAGAGGCGACGTTGCTTTCTTAACCACTGGATTTGCAGTG | |
| | | CTGTGGTTCTCAGAAGAGGCCAGGACAGGTTAATCCACTCACT | |
| | | TGGGAGGCAGGTACCCACATTTCTCTCCAGCTGTTACCTCCAAC | |
| | | CTTTAGTTCCACCGGTCAAAGAAGATATTTTGTTTTGCCTTGTC | |
| | | CTTTGCTAAAGTGATGGTACTTGTTTGCTTTCTCTCTGACAGAA | |
| | | AATGGCAGCTTTGGTTGTTCCCTCTTCCTGCCAATTCCTGTTGTT | |
| | | TTCAGTCATTTTAATTTTAGCCTTCTTCTGGAAGGTGCGAGTGG | |
| | | TATCTCAGCATGATTTCCTATTTCCCTCATGAAAAATGATATCA | |
| | | AATACTTTAGAGTTTCTTCATCAAACTTTTGTGTTTCTTCTCTTG | |
| | | TGCATTTTCTATTCAAGTCCTATGCCTACTTTTAAAAATTTCTTT | |
| | | ACATTTTTATTGATAAATAAAATGTAAGCATTTTAATGCTTAAT | |
| | | GAAATGCTTAATGTTGTGAGCATTTCTATATAAACTAGAGGCA | |
| | | AGTTTTTTTCAGTTGAATGTTATTAATAATTTTGAACAGTCTCCT | |
| | | CATTTTATCACTGGTGTCTTTTGATGAGCAGAGTTTTCATGTTT | |
| | | AATGAAAAGTAATCTACACTTTTTTTCTTTTTTCATTAGCATTTT | |
| | | AATTTGCTACCCAAAACATTCTTGACTACTCCTATTTCATGAAG | |
| | | CTATTCTTCTGTTTTCTCTTAAATGACTCAGCTTTTCACATTTTG | |
| | | ACTTAGCTTTTACATTTCTTGACTTAACTTTTACATTAT | |
| SB09762 | LNCARO | AAGGAGTCATAGAGAAAGGCTTTTCATCAGTCATGGACCTAAA | 416 |
| | Dv5 | AGCGTGCTCTGTAATGGTGGACTTCAGACAGAGATAGAGAGAT | |
| | | CCTAGATGAGTCATCCTGGGATGCAGGATGCAGTTGCTTCTTTT | |
| | | GTTTTGAGAATGCTTGGCTGCTTGCTCCATTCCTACTCATCAGT | |
| | | AGAAGCTTGTCTTAGTGTTTCATTTTCAAGGACCTCTTGATATA | |
| | | AACTACGGTTTGAAGGACATTCCGTTCCTAAGTGGGACAGGAG | |
| | | CAGAGCCTGGGCTGCCATGACCAGTTCTGACATATCTCAGGTT | |
| | | GCTGCATTACCAGAGCATTCCAAAGTTATTCCTGAGAACTACA | |
| | | AGTGAGAAAGGTTGGAAAACTGGGTCAGTTCAAGGCCTCCCAA | |
| | | AGAACCATCCTGCAGCTATCACGAGATATGTCTGTCTGTCTGTC | |
| | | TCTTAAGTAGAGAAGGCAGAGATTAAAAATAAAAAAGATTGG | |
| | | AACAACAAAGATATAGGGAAAAATAATTTTGGAAGCTACCTTT | |
| | | GTGAAATCTGTAGAAAGATGTAGAGAAGAGGAGCTGGTAACA | |
| | | GCCATGTCTGGACTCCCGGTCACCCTATTACCACAACCTGTTAC | |
| | | CCCAACCCCATCACTTCACATAAGAAGCAAAGTTCATGCTAGC | |
| | | CTAGTAGCCCATTTCCTCCAAGACAATTCCCATCAAGCCTGGCC | |
| | | AGGTGGCTCAGCCCAGACCGAAGCATGGAGGATGTGCAGTGTG | |
| | | AAAAAGCACAGACTGAGATGACCATGCAGAAAAGATTTGTAT | |
| | | AAGAAAGCTGGGGCCACCCCTGTAGCCTTGTTTTTATAATACTT | |
| | | TCCTGTGTCAAAAAAATTAATGAAGTTAATTTAGAATGAGAAA | |
| | | TGATACATAATGCCTATAGGTATCTCTGAGAAGATTGCTACAG | |
| | | AGACTAAGAGGATCATAGACCTTCCTTCTGGAATGTTCCTGTTC | |
| | | CTATCTGTAGGATGAATGAGTGGAATCAGTGACTCAGACAAAG | |
| | | CCACTGTTGCACAACTGTATGCATGCACAGTGAACTCTGCAGA | |
| | | CTCTGCAGATGGTTCCTGTAAATAGCGCTTAAAACCACAGTGG | |
| | | AAAAGCCACATGCTCCTGATACACTTGGGGGGACATAAAGGGA | |
| | | AACCCACACACCCAAGGAGGAGGGCTCTGGCCATGCAAGGCT | |
| | | GAAGGCCCAGTGTACTGATATGAAAACTGAAGCTCAGACAGA | |
| | | AGAGGCGACGTTGCTTTCTTAACCACTGGATTTGCAGTGCTGTG | |
| | | GTTCTCAGAAGAGGCCAGGACAGGTTAATCCACTCACTTGGGA | |
| | | GGCAGGTACCCACATTTCTCTCCAGCTGTTACCTCCAACCTTTA | |
| | | GTTCCACCGGTCAAAGAAGATATTTTGTTTTGCCTTGTCCTTTG | |
| | | CTAAAGTGATGGTACTTGTTTGCTTTCTCTCTGACAGAAAATGG | |
| | | CAGCTTTGGTTGTTCCCTCTTCCTGCCAATTCCTGTTGTTTTCAG | |
| | | TCATTTTAATTTTAGCCTTCTTCTGGAAGGTGCGAGTGGTATCT | |
| | | CAGCATGATTTCCTATTTCCCTCATGAAAAATGATATCAAATAC | |
| | | TTTAGAGTTTCTTCATCAAACTTTTGTGTTTCTTCTCTTGTGCAT | |
| | | TTTCTATTCAAGTCCTATGCCTACTTTTAAAAATTTCTTTACATT | |
| | | TTTATTGATAAATAAAATGTAAGCATTTTAATGCTTAATGAAAT | |
| | | GCTTAATGTTGTGAGCATTTCTATATAAACTAGAGGCAAGTTTT | |
| | | TTTCAGTTGAATGTTATTAATAATTTTGAACAGTCTCCTCATTTT | |
| | | ATCACTGGTGTCTTTTGATGAGCAGAGTTTTCATGTTTAATGAA | |
| | | AAGTAATCTACACTTTTTTTCTTTTTTCATTAGCATTTTAATTTG | |
| | | CTACCCAAAACATTCTTGACTACTCCTATTTCATGAAGCTATTC | |
| | | TTCTGTTTTCTCTTAAATGACTCAGCTTTTCACATTTTGACTTAG | |
| | | CTTTTACATTTCTTGACTTAACTTTTACATTATGCTCTTA | |
| SB09763 | LNCARO | CATCCTGGGATGCAGGATGCAGTTGCTTCTTTTGTTTTGAGAAT | 417 |
| | Dv6 | GCTTGGCTGCTTGCTCCATTCCTACTCATCAGTAGAAGCTTGTC | |
| | | TTAGTGTTTCATTTTCAAGGACCTCTTGATATAAACTACGGTTT | |
| | | GAAGGACATTCCGTTCCTAAGTGGGACAGGAGCAGAGCCTGGG | |
| | | CTGCCATGACCAGTTCTGACATATCTCAGGTTGCTGCATTACCA | |
| | | GAGCATTCCAAAGTTATTCCTGAGAACTACAAGTGAGAAAGGT | |
| | | TGGAAAACTGGGTCAGTTCAAGGCCTCCCAAAGAACCATCCTG | |
| | | CAGCTATCACGAGATATGTCTGTCTGTCTGTCTCTTAAGTAGAG | |
| | | AAGGCAGAGATTAAAAATAAAAAAGATTGGAACAACAAAGAT | |
| | | ATAGGGAAAAATAATTTTGGAAGCTACCTTTGTGAAATCTGTA | |
| | | GAAAGATGTAGAGAAGAGGAGCTGGTAACAGCCATGTCTGGA | |
| | | CTCCCGGTCACCCTATTACCACAACCTGTTACCCCAACCCCATC | |
| | | ACTTCACATAAGAAGCAAAGTTCATGCTAGCCTAGTAGCCCAT | |
| | | TTCCTCCAAGACAATTCCCATCAAGCCTGGCCAGGTGGCTCAG | |
| | | CCCAGACCGAAGCATGGAGGATGTGCAGTGTGAAAAAGCACA | |
| | | GACTGAGATGACCATGCAGAAAAGATTTGTATAAGAAAGCTGG | |
| | | GGCCACCCCTGTAGCCTTGTTTTTATAATACTTTCCTGTGTCAA | |
| | | AAAAATTAATGAAGTTAATTTAGAATGAGAAATGATACATAAT | |
| | | GCCTATAGGTATCTCTGAGAAGATTGCTACAGAGACTAAGAGG | |
| | | ATCATAGACCTTCCTTCTGGAATGTTCCTGTTCCTATCTGTAGG | |
| | | ATGAATGAGTGGAATCAGTGACTCAGACAAAGCCACTGTTGCA | |
| | | CAACTGTATGCATGCACAGTGAACTCTGCAGACTCTGCAGATG | |
| | | GTTCCTGTAAATAGCGCTTAAAACCACAGTGGAAAAGCCACAT | |
| | | GCTCCTGATACACTTGGGGGGACATAAAGGGAAACCCACACAC | |
| | | CCAAGGAGGAGGGCTCTGGCCATGCAAGGCTGAAGGCCCAGT | |
| | | GTACTGATATGAAAACTGAAGCTCAGACAGAAGAGGCGACGTT | |
| | | GCTTTCTTAACCACTGGATTTGCAGTGCTGTGGTTCTCAGAAGA | |
| | | GGCCAGGACAGGTTAATCCACTCACTTGGGAGGCAGGTACCCA | |
| | | CATTTCTCTCCAGCTGTTACCTCCAACCTTTAGTTCCACCGGTC | |
| | | AAAGAAGATATTTTGTTTTGCCTTGTCCTTTGCTAAAGTGATGG | |
| | | TACTTGTTTGCTTTCTCTCTGACAGAAAATGGCAGCTTTGGTTG | |
| | | TTCCCTCTTCCTGCCAATTCCTGTTGTTTTCAGTCATTTTAATTT | |
| | | TAGCCTTCTTCTGGAAGGTGCGAGTGGTATCTCAGCATGATTTC | |
| | | CTATTTCCCTCATGAAAAATGATATCAAATACTTTAGAGTTTCT | |
| | | TCATCAAACTTTTGTGTTTCTTCTCTTGTGCATTTTCTATTCAAG | |
| | | TCCTATGCCTACTTTTAAAAATTTCTTTACATTTTTATTGATAAA | |
| | | TAAAATGTAAGCATTTTAATGCTTAATGAAATGCTTAATGTTGT | |
| | | GAGCATTTCTATATAAACTAGAGGCAAGTTTTTTTCAGTTGAAT | |
| | | GTTATTAATAATTTTGAACAGTCTCCTCATTTTATCACTGGTGT | |
| | | CTTTTGATGAGCAGAGTTTTCATGTTTAATGAAAAGTAATCTAC | |
| | | ACTTTTTTTCTTTTTTCATTAGCATTTTAATTTGCTACCCAAAAC | |
| | | ATTCTTGACTACTCCTATTTCATGAAGCTATTCTTCTGTTTTCTC | |
| | | TTAAATGACTCAGCTTTTCACATTTTGACTTAGCTTTTACATTTC | |
| | | TTGACTTAACTTTTACATTATGCTCTTATATTAATACTGAGTTA | |
| | | ATTTGCATTGATGGTGTGTGGGAGTATTCAAGGCAGATTATCTC | |
| | | TTTTAATAAATCTACAGTGATCTAAGCAATATTTGTA | |
| SB09785 | ID3 | GAAGTCTGCAAGATAGGAAAAGAAGGATCTTCTCTTCGGTTAT | 418 |
| | | TCAGGAGTTCCTACAGCCATTATTTTGCCTTTCCTGACCAAATA | |
| | | GCTGGCACTTGACGGCTGTAGCATGTAAAGACTGACTTGCCCA | |
| | | CTCACAGGTTTTCTTCCTCTTTTCTTACTGAAGAGCCTGTCCTTG | |
| | | AAAAAAACATTCCAGGAAGAAATCTTTCTTGGGCCACAGAGCC | |
| | | CTGTGGTTTGATGGAAAAGAACGAGGCTTCAAGACTGGCTCTG | |
| | | CTATTACTTGTAGTGTGACATTGGGCATGTCTTAAATGCCTTCC | |
| | | TTGGGCCCCTCAGCATCTTTCACCTACATATCCAAATTTCACAC | |
| | | AACCTTGGACTTCTACAATTGGAAAGGATTTTAAATAATCACTT | |
| | | AGTACCATCGTCTATTTTACTCACAAGGCAATTAAAGCCAGAG | |
| | | TACCTGGGCTTGTTCAAGATTACATGACAAATTATTGTCAGGGC | |
| | | TAGCATGATGAACCCAGGGCTCTCAATTCCCAGTCAGTGTCCTT | |
| | | TTCAACCGTTTTGCGACTCTCTTTGGCTCTGAGCCCCCCTTGTCC | |
| | | TGTTAATCAGTGCCACACATTATCGAACTCTTGTTTCCTGTGTT | |
| | | AATCTTGTCTCCCCAAATTGTGAGATTCTTGAAAGCAGGAGCC | |
| | | ACTGTTTTATGCGGAGGGACTCCATAAATATTCACCAATGATTC | |
| | | ATTAGGAAAGCAACTATTGGGGGAATCCCACTTGGTCATTTCC | |
| | | AAAATGGAAAGCAGCATCTTGGAAGTGCAGGTCCCCAAATGTC | |
| | | TAGGGAGGTGACAAGAAGGCAAGCAAAGAATCAAAGAATACT | |
| | | GAGCACAGTGTCCTGTTGCAATACAGGACTTAGTTTGCCGGCC | |
| | | CTCTGGTTCCTTTCCTGGTCTTTCAGATGGAGCTGGAGCTACTT | |
| | | CAGCAGGGCTAAGAATAATCAAGTCCAGGTGGCCTGCCCTTCC | |
| | | ATAGCAGAAAGGGACAGACAATAGAGCAATGAAATTACCACT | |
| | | GTTTTCTACACAGAGGTGTGTGGAAAGTGAAGAGAGGGGAAGT | |
| | | TAAGAAATTTGCTGACCCAATTGGGTGGGGGGGGGCTCTTAAA | |
| | | AAGGTTTATGTAGAAATTGTCCCATCTGTGCCTCCATATTTAAA | |
| | | ACCTTGTCCAAAATTCTAATTGAACTCACAAAGCACAACTGTG | |
| | | CATATATTTAGTTCGTTATGGAGTTTATGGGGCAGAGGGTCCA | |
| | | GGCTCCTTTTTTCCCCATGCAAAAAGCATGGGCTCAAGCTTTCT | |
| | | TCTTTTCCCCTGTTGCTCAAATAAATAGTGTTCTTTGCTCAAAC | |
| | | CCCCTTTCCCTCCTCCTTCTGCAATCTCAGCGCCTAGCGCAAAT | |
| | | CTGTTTTCTTCATTGTAACCTCAGCTTCACCGCAATTAATTTTTT | |
| | | TTCCCTCTGGTCACAAGATAATTCCTGACGCCAGTGAGTCTGGA | |
| | | GGTCAGACGAACAGCAAATTGGGGAACAAGGCGGCACTAATT | |
| | | CCTTACAAGTTTCCCTTGAAAAATCTTTCGCTTAAAAAAAACGG | |
| | | GGGGTGGGGGGAGCTTCTTTGCTGTTCAGGGATTTATGACCTC | |
| | | GGAGGAGCTGTGGCTCGAACCAGTGTTGGGCTAAAGGCGGACT | |
| | | GGCAGGGGGCAGGGAAGCTCAAAGATCTGGGGTGCTGCCAGG | |
| | | AAAAAGCAAATTCTGGAAGTTAATGGTTTTGAGTGATTTTTAA | |
| | | ATCCTTGCTGGCGGAGAGGCCCGCCTCTCCCCGGTATCAGCGC | |
| | | TTCCTCATTCTTTGAATCCGCGGCTCCGCGGTCTTCGGCGTCAG | |
| | | ACCAGCCGGAGGAAGCCTGTTTGCAATTTAAGCGGGCTGTGAA | |
| | | CGCCCAGGGCCGGCGGGGGCAGGGCCGAGGCGGGCCATTTTG | |
| | | AATAAAGAGGCGTGCCTTCCAGGCAGGCTCTATAAGTGACCGC | |
| | | CGCGGCGAGCGTGCGCGCGTTGCAGGTCACTGTAGCGGGACTT | |
| | | CTTTTGGTTTTCTTTCTCTTTGGGGCACCTCTGGACTCACTCCCC | |
| | | AGC | |
| SB05148 | MRC1 | GAATCGCTTGAACCCGGGAGGTGGAGGTTGCAGTGAGCCCAGA | 419 |
| | | TTGCACCACTGCACTCCAGCCTGGGCGACAGAGCAAGACTCTG | |
| | | TCTCAAAGAAACAAACCAACCCTGAATGTGATTATATACATAA | |
| | | TTCAATTAAATGTATTTGCTTCTGAAATATATATAAATGTAAAT | |
| | | TAGGCAGTCACTTTTGTATATGATTTATTTATATTTGAAAGCCA | |
| | | CAAATGACCCATTTAAACTATTATTTTCATAAGCCAGTGAAAC | |
| | | AATGTCTGAGAAACATTTTTGTTTTGTCTGTTCTGTTCTATAACC | |
| | | ATCATTTTTTTTTTCAGTCATGTACAGCCTTAGTGACAAAGAAA | |
| | | CTTTGGTCCTCTGTCCTACATTTTCACTATCTTTTTCCCTCCGGT | |
| | | CAGGATAATCTCAAATTTACATGTTAAAAACAATCAGTAAGAG | |
| | | AACTACATCACATTTCTAATAGGATGGAAACTTTTCAACTTTAT | |
| | | CACAAAGACAACGAATGTGGAGGCTTTCCGTTTGAAGATAAAA | |
| | | CTATTCATTTAAAAAATTTTAAAAATTACAATGTTTCCAGTAGC | |
| | | TTCTTTTTGAATTACTAACATATTCCACACTCTAGTAACGGTTT | |
| | | GGCCAGCTAATCGTTAGTTTCTGCTTTAAAATGTTCTAAATTCC | |
| | | TGTTCTACTTTTGAAAAATGACAACATAAATGTTTGGAGGGTTA | |
| | | TTTTCTGCTTAATGAAAGATCTAGAAACATATTTTATTCTAAGA | |
| | | AAGAATTCCACTTGCCTTTAAATAAAGATATACCTTTTGACCAA | |
| | | ACAATCAGATTTTCTTTTTCTTTTTTTTCTTTTCTTTTTTTTTTTT | |
| | | GAGATGGAGTTTCGCGTCTGTCGCCCAGGCTGGAGTGTAGTGG | |
| | | TGCGATCCTGACTCACTGTAACTTCCACTTCCCAGGTTCAAACG | |
| | | ATTCTGTTGCCTCAGCCTCCTGAGTAGCTGGGCTTACAGGTGTG | |
| | | CATGATCACACCCGGCTAACTTTTGTATTTTTAGTAGAGACGGG | |
| | | TTTTTGCCATGTTGACCAGGCTGGTTTCAAACTCCTGACCTCGG | |
| | | GTGATCTGACTGCCTCGGCCTCCCAAACTGCTGGGATTGCAGG | |
| | | CGTGAGCCATTGTGCCTGGCCAGATTTTCTTTTTCTAGCAAGGG | |
| | | GACCCACTTAAACTTGAAGAGGACCGGGATGGTTGAGGCTGGG | |
| | | CAGCAAGGCTTTACTGCAAATCCTTTACCACTGTTTTTTCTGGC | |
| | | TTTCTAGAGAACGTTCTAGCAAAAGGTTTCTAGAACTTTCTCCT | |
| | | TCCTGGCCTGACTGACATTCCCTCTTAGGTGTAGCCTCCTTTTC | |
| | | ACTTTTCTTCTGCCTGGAGGAAATGAAGCTCCACGGAACTTTCT | |
| | | GTTGAAACTTTCCAAGAAAAAAAAGAAAGGCTCTAAGCACTGA | |
| | | ATGTGGAAACTGAAGGGGATGAGCTTCAACTCTGAAGTGTTTC | |
| | | CAGCGTAAAACTGTCCTTTCCAGGGCCCGTGTGGCTGTCACTTC | |
| | | AGAGTGGAGGTTGTCTGCTGAGGGACCCCTGACTCAGCTGCTT | |
| | | CCCAGGGGAAGCTCCGTCTTCCGGCACAGGTAATGGCCTGCAG | |
| | | CTTGATCTCCACCCAGCCCCATCTGAGCAGGCCGGGAGCTCCC | |
| | | AGGCTGTTTCACTTCTCTCCTTCCTGACTCCTCACCATCACCATC | |
| | | GCCCTCTCTCCTCCCCACCCCGCCACTCCTCTCCCACACGTGTC | |
| | | CCTTTCTCCCCTTCCTCTGCGTCTGCTCTTCTCAGAAGTTAGCTT | |
| | | ACGAAGCAAAGTTGTTACTTTGAATTCCTGTTTTTCCAGCCACC | |
| | | CTCATGTGACAGGATGTCTCCTCAGTAGAGGCTTTCCCTAAATT | |
| | | CAGGAGCCCTTTAAAAGGGAGGGCTTCCTCTGTAGTTCTTTTCA | |
| | | GCTGGGCAGCTCTGGGAACTTGGATTAGGTGGAGAGGCAGTTG | |
| | | GGGGGCCTCGTTGTTTTGCGTCTTAGTTCCGCCCTCCTGTCCAT | |
| | | CAGGAGAAGGAAAGGATAAACCCTGGGCC | |
| SB12087 | IDO1_1_ | GCTCTTCTAAAAATATGCGAAATGAGGTTTTTAGGGAGGTGTA | 456 |
| | +_m_27 | GGTATGGCTGAAGAAAATCAAGGTGAATGAAGACAAGATCAA | |
| | | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCA | |
| | | ACTGATTCCCAAAGTATTAGCCTCATGAATCATGTAGTCATAA | |
| | | GAAACACAGTCATTGTATTCTCTTTGCTGTATAATTTTGGTTTC | |
| | | AGTTTTCCTTACATTTCCTATTCAAGGAACATTTTCCTGTAAAA | |
| | | TGACAGGTTGAAGAAAACAGCCATAATTTAGTAGAGAATAGCG | |
| | | CGAGAGCTATTCTAGACTGTAACGAAAGCCATATGCTATCACA | |
| | | ATTTAATTTATTTCAAGTACTAATAAGCTGATGACAAAACAGC | |
| | | GATGTCTTTTAGTTTACTCACACGAACTATTTCTCTTTTCTCCTT | |
| | | TTGATCATCTAGAGGAACGGGCAACTTGGTTTCTTCTTTAGCTT | |
| | | CCTTGTTCTCATTAAGATTGAACAATGCCTCTAAAGTGAACCAC | |
| | | AGACTTGCATGCAAGCTGAAAACCTTTACCAAATGCAGTCTTA | |
| | | ATTTGTACTTTGAGAAAAACATTTTCAAGGTATAACTAAATGA | |
| | | GAGTTTAGGACTGCAGCCTTCATTTTCATTCAAAGATTTAAAAG | |
| | | TTTCCATAAAGTAAAATGTTCTTCTCCGGCCACCTGTTTTCATA | |
| | | GTTCTGTGTTTTCCTTCAGGCCTTTCTGGCTTCCTATATGGCAGT | |
| | | AAGAAAATGATGTGCTTAATGATTACAAATTTCATATGGAATA | |
| | | CGAACTTTCAGTTTGTACATATGATGCACAGAGATGCTTTTGTG | |
| | | GTTTTATTGGTTTTCATATTACAAACAAAGAAACTAGAAAATG | |
| | | AAACCATTCCAAAAGTGGAAGTAATTTCTCACTGCCCCTGTGA | |
| | | TAAACTGTGGTCACTGGCTGTGGCAGCAACTATTATAAGATGC | |
| | | TCTGAAAACTCTTCAGACACTGAGGGGCACCAGAGGAGCAGAC | |
| | | TACAAGA | |
| SB12090 | IDO1_1_ | GCTCTTCTAAAAATATGCGAAATGAGGTTTTTAGGGAGGTGTA | 457 |
| | +_m_30 | GGTATGGCTGAAGAAAATCAAGGTGAATGAAGACAAGATCAA | |
| | | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCtt | |
| | | ctaagtccaattcacgacaaGAGTAGCTGGGATTACAGGCACATGCCACCA | |
| | | TGCCCAGCTAATTTTTGTATTTTCAATAGAGACTGGGTTTCACC | |
| | | ATGTTGCCCAGGCTGGTCTTCAACTCCTGACCTCAAGTGATCCG | |
| | | CCCTCCTCAGCTTCCCAAAGTGCgccttcataaGGCGTGAGCCACCA | |
| | | CACCGGGGGGTAGGATAGATTTAGTGAGATGACTGGATAAACG | |
| | | GAATCAAGAAAAAGCTTTGTCAAAAACTTATGCTTCTTAAAAA | |
| | | CTTAATCCTGGGACAGAATCATCTAAAACGTTGTTCCATGTCTT | |
| | | CACTTTGACTCACCCATAAAAACTTCAAGTACAAAGAATGAAA | |
| | | AATAACCACATATTTTCTAATGCTCAATAtctcgctaataggagtaagataca | |
| | | TATAGATATGATAGGTCTTCAttctgctgcaagacctatactatAACTGATTC | |
| | | CCAAAGTATTAGCCTCATGAATCATGTAGTCATAAGAAACACA | |
| | | GTCATTGTATTCTCTTTGCTGTATAATTTTGGTTTCAGTTTTCCT | |
| | | TACATTTCCTATTCAAGGAACATTTTCCTGTAAAATGACAGGTT | |
| | | GAAGAAAACAGCCATAATTTAGTAGAGAATAGCGCGAGAGCT | |
| | | ATTCTAGACTGTAACGAAAGCCATATGCTATCACAATTTAATTT | |
| | | ATTTCAAGTACTAATAAGCTGATGACAAAACAGCGATGTCTTT | |
| | | TAGTTTACTCACACGAACTATTTCTCTTTTCTCCTTTTGATCATC | |
| | | TAGAGGAACGGGCAACTTGGTTTCTTCTTTAGCTTCCTTGTTCT | |
| | | CATTAAGATTGAACAATGCCTCTAAAGTGAACCACAGACTTGC | |
| | | ATGCAAGCTGAAAACCTTTACCAAATGCAGTCTTAATTTGTACT | |
| | | TTGAGAAAAACATTTTCAAGGTATTTTATCCTTTTCTCCAACTT | |
| | | TTGACATATTACAAAGTACCCAAATATGCCAGACTGTTGCCTC | |
| | | ATCAGCCCCCCGCAGTCAGGTACAGTTAGATGCAAGGCAATCT | |
| | | TCCTAAAAGTTACTTATTAGAGATGTGAGAAGGGCAAATGCTA | |
| | | TCATTGGAAAAACTGACAAAAGTCCCAATAGGAcgagttcgataataca | |
| | | ctGTTACTATGTTTCTAATTTTTCATGTGCTTCTATTTTTTTCCTA | |
| | | CTTCAGAGCCATTGACTAATAGTTGAGTATAACACAGGTTGTG | |
| | | TTTCCGGGCTGCTGAAACATGACACTAATATTTTCAAAGAACT | |
| | | GTGGAAGCCTAAAAGGtgtctgtataaagATAACTAAATGAGAGTTTA | |
| | | GGACTGCAGCCTTCATTTTCATTCAAAGATTTAAAAGTTTCCAT | |
| | | AAAGTAAAATGTTCTTCTCCGGCCACCTGTTTTCATAGTTCTGT | |
| | | GTTTTCCTTCAGGCCTTTCTGGCTTCCTATATGGCAGTAAGAAA | |
| | | ATGATGTGCTTAATGATTACAAATTTCATATGGAATACGAACTT | |
| | | TCAGTTTGTACATATGATGCACAGAGATGCTTTTGTGGTTTTAT | |
| | | TGGTTTTCATATTACAAACAAAGAAACTAGAAAATGAAACCAT | |
| | | TCCAAAAGTGGAAGTAATTTCTCACTGCCCCTGTGATAAACTGT | |
| | | GGTCACTGGCTGTGGCAGCAACTATTATAAGATGCTCTGAAAA | |
| | | CTCTTCAGACACTGAGGGGCACCAGAGGAGCAGACTACAAGA | |
| SB12091 | IDO1_1_ | GCTCTTCTAAAAATATGCGAAATGAGGTTTTTAGGGAGGTGTA | 458 |
| | +_m_31 | GGTATGGCTGAAGAAAATCAAGGTGAATGAAGACAAGATCAA | |
| | | TTGAGAATGTAGTTTCAGAAATAGCAAAGAAGCCAAAGTTTGA | |
| | | GGAAGTTAAGTGGCTAGGGATAACATTGAGGCACTAAAGCATT | |
| | | ATTGGTTCTGCAGTCAAGGGTAGGATAGATTGTTTTTTTTTTTTT | |
| | | TGAGACGGAGTCTCACTCTGCTGCCCAGGCTGGAGTGCAATGG | |
| | | TGCAATCTTGGCTCACTGCAACCTCTGCCTCTCGGGCTCAAGCG | |
| | | AGTAGCTGGGATTACAGGCACATGCCACCATGCCCAGCTAATT | |
| | | TTTGTATTTTCAATAGAGACTGGGTTTCACCATGTTGCCCAGGC | |
| | | TGGTCTTCAACTCCTGACCTCAAGTGATCCGCCCTCCTCAGCTT | |
| | | CCCAAAGTGCGGCGTGAGCCACCACACCGGGGGGTAGGATAG | |
| | | ATTTAGTGAGATGACTGGATAAACGGAATCAAGAAAAAGCTTT | |
| | | GTCAAAAACTTATGCTTCTTAAAAACTTAATCCTGGGACAGAA | |
| | | TCATCTAAAACGTTGTTCCATGTCTTCACTTTGACTCACCCATA | |
| | | AAAACTTCAAGTACAAAGAATGAAAAATAACCACATATTTTCT | |
| | | AATGCTCAATATATAGATATGATAGGTCTTCAAACTGATTCCCA | |
| | | AAGTATTAGCCTCATGAATCATGTAGTCATAAGAAACACAGTC | |
| | | ATTGTATTCTCTTTGCTGTATAATTTTGGTTTCAGTTTTCCTTAC | |
| | | ATTTCCTATTCAAGGAACATTTTCCTGTAAAATGACAGGTTGAA | |
| | | GAAAACAGCCATAATTTAGTAGAGAATAGCGCGAGAGCTATTC | |
| | | TAGACTGTAACGAAAGCCATATGCTATCACAATTTAATTTATTT | |
| | | CAAGTACTAATAAGCTGATGACAAAACAGCGATGTCTTTTAGT | |
| | | TTACTCACACGAACTATTTCTCTTTTCTCCTTTTGATCATCTAGA | |
| | | GGAACGGGCAACTTGGTTTCTTCTTTAGCTTCCTTGTTCTCATT | |
| | | AAGATTGAACAATGCCTCTAAAGTGAACCACAGACTTGCATGC | |
| | | AAGCTGAAAACCTTTACCAAATGCAGTCTTAATTTGTACTTTGA | |
| | | GAAAAACATTTTCAAGGTATTTTATCCTTTTCTCCAACTTTTGA | |
| | | CATATTACAAAGTACCCAAATATGCCAGACTGTTGCCTCATCA | |
| | | GCCCCCCGCAGTCAGGTACAGTTAGATGCAAGGCAATCTTCCT | |
| | | AAAAGTTACTTATTAGAGATGTGAGAAGGGCAAATGCTATCAT | |
| | | TGGAAAAACTGACAAAAGTCCCAATAGGAGTTACTATGTTTCT | |
| | | AATTTTTCATGTGCTTCTATTTTTTTCCTACTTCAGAGCCATTGA | |
| | | CTAATAGTTGAGTATAACACAGGTTGTGTTTCCGGGCTGCTGA | |
| | | AACATGACACTAATATTTTCAAAGAACTGTGGAAGCCTAAAAG | |
| | | GATAACTAAATGAGAGTTTAGGACTGCAGCCTTCATTTTCATTC | |
| | | AAAGATTTAAAAGTTTCCATAAAGTAAAATGTTCTTCTCCGGCC | |
| | | ACCTGTTTTCATAGTTCTGTGTTTTCCTTCAGGCCTTTCTGGCTT | |
| | | CCTATATGGCAGTAAGAAAATGATGTGCTTAATGATTACAAAT | |
| | | TTCATATGGAATACGAACTTTCAGTTTGTACATATGATGCACAG | |
| | | AGATGCTTTTGTGGTTTTATTGGTTTTCATATTACAAACAAAGA | |
| | | AACTAGAAAATGAAACCATTCCAAAAGTGGAAGTAATTTCTCA | |
| | | CTGCCCCTGTGATAAACTGTGGTCACTGGCTGTGGCAGCAACT | |
| | | ATTATAAGATGCTCTGAAAACTCTTCAGACACTGAGGGGCACC | |
| | | AGAGGAGCAGACTACAAGA | |
| SB12093 | UBD_1_-_ | GGTTTGTTGTTCATATGTTTTCTTGAAAGGGCACTATTTCCCAG | 459 |
| | m_20 | AATCCAGGTCATCTCTGGGTAGGGAATCCCCTGAATTAGTTTTT | |
| | | TCTGAGAGTATATTAGATTTGCCCTCACAGTAACCCCATGCTGG | |
| | | AAATACCCAACTCCAGTAGCAACTAGTTCTAGAGGGTATATAA | |
| | | TGGGGGCCAACGCGTaccggtgtcGCCACC | |
| SB12094 | UBD_1_-_ | GGTTTGTTGTTCATATGTTTTCTTGAAAGGGCACTATTTCCCAG | 460 |
| | m_21 | AATCCAGGTCATCTCTGGGTAGGGAATCCCCTGAATTAGTTTTT | |
| | | TCTGAGAGTATATTAGATTTGCCCTCACAGTAACCCCATGCTGG | |
| | | AAATACCCAACTCCAGTAGCAGGTTTGTTGTTCATATGTTTTCT | |
| | | TGAAAGGGCACTATTTCCCAGAATCCAGGTCATCTCTGGGTAG | |
| | | GGAATCCCCTGAATTAGTTTTTTCTGAGAGTATATTAGATTTGC | |
| | | CCTCACAGTAACCCCATGCTGGAAATACCCAACTCCAGTAGCA | |
| | | GGTTTGTTGTTCATATGTTTTCTTGAAAGGGCACTATTTCCCAG | |
| | | AATCCAGGTCATCTCTGGGTAGGGAATCCCCTGAATTAGTTTTT | |
| | | TCTGAGAGTATATTAGATTTGCCCTCACAGTAACCCCATGCTGG | |
| | | AAATACCCAACTCCAGTAGCAACTAGTTCTAGAGGGTATATAA | |
| | | TGGGGGCCAACGCGTaccggtgtcGCCACC | |
| SB12095 | UBD_1_-_ | GGTTTGTTGTTCATATGTTTTCTTGAAAGGGCACTATTTCCCAG | 461 |
| | m_22 | AATCCAGGTCATCTCTGGGTAGGGAATCCCCTGAATTAGTTTTT | |
| | | TCTGAGAGTATATTAGATTTGCCCTCACAGTAACCCCATGCTGG | |
| | | AAATACCCAACTCCAGTAGCActgtaatatcatccgctctttaCTGAAATAAC | |
| | | ATAATAATATAGGAAGAATACAAGGACTATAGAAATACAGATT | |
| | | AGTGTTTGAACCCTTGCTTACCAGCTACTACTAATATGATTGTG | |
| | | GATGAGGTAGCTTCTTACTTATTAACGGGGATACTAATAGAGG | |
| | | TGGTTCCTtgatcggccaatatttATGATTTTTTATTTAATAGTGATACCA | |
| | | AAGCAATACATACTCAGTAGAAACCTACTTCAAGTTCCCATAA | |
| | | AATCATCTGCTTTTCACTTTCAGTACAGTATTTAATAACTTAAA | |
| | | TGAGATATTTCACACTTCAGTAGTAAATACACTTTTTGTTAGAT | |
| | | AATTTTGTCCAACTGTATGCTAATGTAAGTGTTCTGAGCATGTT | |
| | | TAAGGCAGGTTAGGTTAAGCTATGATGTTTGGTGGGTTAGGTG | |
| | | TATTAAATGCATTTCTGATTTTGGATATTTTCAGTGTACAATGG | |
| | | GTTTACAGGGATGTAACCCCATCATAAGTGAAGGAGCACCTGT | |
| | | ACTTACTTCATTAAAATGCTGAAACAGTAAATAAGGTAACATT | |
| | | TAATAATATGTTGTGCAGTTCTTGAAATTTAAGTACTCACCAAA | |
| | | TATTACTTTTCCTTTTTTTGTTATTTACTTACTTTTCATTCATTTA | |
| | | TTAATTCATTTGTGCATTTAGTAAACATTTATAAATTATTTCCTG | |
| | | TGCCTGACAGCATGCTGGAACAGTGCTAAAGATACAAGTTAAT | |
| | | TAAGACACAATCACGACCCCCAAGATTCCTACTCTTTTCTAAAG | |
| | | ATTACAGACAAGCAGACGATGCTATTGTTGAAGAAACATGCTC | |
| | | TGAGAGGCATTTGAAGGAAGTGTAGAGGATAGAAGATGGACA | |
| | | CATAACCCAGGATGGGGAGGAAAAGAGTTAGGGAAGGCTTTTT | |
| | | GACGAAGATACTGTTTACACCGTGTGTTCTTATAAATTCATGGT | |
| | | GGTGGGGATAGAGTTGGAGGAAAAGGCATGCTCAGTGGCGTG | |
| | | GAGATGGCAGAGAGATTGGGGTGTTCAAGGATATGCCGGGAA | |
| | | TTCAAGGAACGAGAATTCCCATAGACACAGACACAGCTAGACA | |
| | | TAGAGATCTGCAGCTTAGGTTTGGGCTGTGGGTATAGATCCAG | |
| | | GTGGCTTCAACAGACAAAGATCTTTCCTGAGAAAAGGGAAAAG | |
| | | TTTTCAACACAGAAAGACCATCCCATGTTTGGAATGAGGTTTG | |
| | | CAAATAGATTGCTTGAGGAGAGAAGTATGTGATCAGAAAGCAT | |
| | | TCTTTGTCTATTAACTCCTGCCCAGCAAAAGTGAAAGAAAATTC | |
| | | ATGGGAGCATGCAAGAACAAAGAGCACAGCAAAGCTGGACAA | |
| | | ACACAGCAATCCAGGCAGGGGATTTCCAACTCAACTCTGGTAT | |
| | | ATAAGCTGCATGCAAAGTCCTTTTTCTGTCTCTGGTTTCTGGCC | |
| | | CCTTGTCTGCAGAG | |
| SB12096 | UBD_1_-_ | TCTCCTTCACCTTCCACCATGAGTAAAAGCTTCTTGAGGCCTetta | 462 |
| | m_23 | cctactaggttaaGGTGCCATACTTCTTGTATAGTTGGAGGAACTATGA | |
| | | ATCAATTAAACactegaattcagaAAATTACCCAGCCTCAAGTATTCCT | |
| | | TTATAGTATGCAAAAGAGACTAAAACAGCAAATGAAAAGAGA | |
| | | TCTGTCTCTGAAAGAACTTATTGattctagccttacagcctaaAAAAGTCAG | |
| | | ATTGACACTGGGTAAGAAGCAAGGAGGTCAGGTCCCAAGATG | |
| | | AAGTCCTGCCTGTGGTCAGCAAAGGGGCACCAAGGTGTCTGGG | |
| | | ACAGTCCTGGCCCTGGCTTTGactctacggaagtagcttgtttaaaacctatagtct | |
| | | cttcggagtcgttctactagtacaaaGGTTTGTTGTTCATATGTTTTCTTGAAAGG | |
| | | GCACTATTTCCCAGAATCCAGGTCATCTCTGGGTAGGGAATCC | |
| | | CCTGAATTAGTTTTTTCTGAGAGTATATTAGATTTGCCCTCACA | |
| | | GTAACCCCATGCTGGAAATACCCAACTCCAGTAGCActgtaatatcatc | |
| | | cgctctttaCTGAAATAACATAATAATATAGGAAGAATACAAGGACT | |
| | | ATAGAAATACAGATTAGTGTTTGAACCCTTGCTTACCAGCTACT | |
| | | ACTAATATGATTGTGGATGAGGTAGCTTCTTACTTATTAACGGG | |
| | | GATACTAATAGAGGTGGTTCCTtgatcggccaatatttATGATTTTTTATT | |
| | | TAATAGTGATACCAAAGCAATACATACTCAGTAGAAACCTACT | |
| | | TCAAGTTCCCATAAAATCATCTGCTTTTCACTTTCAGTACAGTA | |
| | | TTTAATAACTTAAATGAGATATTTCACACTTCAGTAGTAAATAC | |
| | | ACTTTTTGTTAGATAATTTTGTCCAACTGTATGCTAATGTAAGT | |
| | | GTTCTGAGCATGTTTAAGGCAGGTTAGGTTAAGCTATGATGTTT | |
| | | GGTGGGTTAGGTGTATTAAATGCATTTCTGATTTTGGATATTTT | |
| | | CAGTGTACAATGGGTTTACAGGGATGTAACCCCATCATAAGTG | |
| | | AAGGAGCACCTGTACTTACTTCATTAAAATGCTGAAACAGTAA | |
| | | ATAAGGTAACATTTAATAATATGTTGTGCAGTTCTTGAAATTTA | |
| | | AGTACTCACCAAATATTACTTTTCCTTTTTTTGTTATTTACTTAC | |
| | | TTTTCATTCATTTATTAATTCATTTGTGCATTTAGTAAACATTTA | |
| | | TAAATTATTTCCTGTGCCTGACAGCATGCTGGAACAGTGCTAA | |
| | | AGATACAAGTTAATTAAGACACAATCACGACCCCCAAGATTCC | |
| | | TACTCTTTTCTAAAGATTACAGACAAGCAGACGATGCTATTGTT | |
| | | GAAGAAACATGCTCTGAGAGGCATTTGAAGGAAGTGTAGAGG | |
| | | ATAGAAGATGGACACATAACCCAGGATGGGGAGGAAAAGAGT | |
| | | TAGGGAAGGCTTTTTGACGAAGATACTGTTTACACCGTGTGTTC | |
| | | TTATAAATTCATGGTGGTGGGGATAGAGTTGGAGGAAAAGGCA | |
| | | TGCTCAGTGGCGTGGAGATGGCAGAGAGATTGGGGTGTTCAAG | |
| | | GATATGCCGGGAATTCAAGGAACGAGAATTCCCATAGACACAG | |
| | | ACACAGCTAGACATAGAGATCTGCAGCTTAGGTTTGGGCTGTG | |
| | | GGTATAGATCCAGGTGGCTTCAACAGACAAAGATCTTTCCTGA | |
| | | GAAAAGGGAAAAGTTTTCAACACAGAAAGACCATCCCATGTTT | |
| | | GGAATGAGGTTTGCAAATAGATTGCTTGAGGAGAGAAGTATGT | |
| | | GATCAGAAAGCATTCTTTGTCTATTAACTCCTGCCCAGCAAAA | |
| | | GTGAAAGAAAATTCATGGGAGCATGCAAGAACAAAGAGCACA | |
| | | GCAAAGCTGGACAAACACAGCAATCCAGGCAGGGGATTTCCA | |
| | | ACTCAACTCTGGTATATAAGCTGCATGCAAAGTCCTTTTTCTGT | |
| | | CTCTGGTTTCTGGCCCCTTGTCTGCAGAG | |
| SB12097 | UBD_1_-_ | TCTCCTTCACCTTCCACCATGAGTAAAAGCTTCTTGAGGCCTGG | 463 |
| | m_24 | TGCCATACTTCTTGTATAGTTGGAGGAACTATGAATCAATTAAA | |
| | | CAAATTACCCAGCCTCAAGTATTCCTTTATAGTATGCAAAAGA | |
| | | GACTAAAACAGCAAATGAAAAGAGATCTGTCTCTGAAAGAACT | |
| | | TATTGAAAAGTCAGATTGACACTGGGTAAGAAGCAAGGAGGTC | |
| | | AGGTCCCAAGATGAAGTCCTGCCTGTGGTCAGCAAAGGGGCAC | |
| | | CAAGGTGTCTGGGACAGTCCTGGCCCTGGCTTTGGGTTTGTTGT | |
| | | TCATATGTTTTCTTGAAAGGGCACTATTTCCCAGAATCCAGGTC | |
| | | ATCTCTGGGTAGGGAATCCCCTGAATTAGTTTTTTCTGAGAGTA | |
| | | TATTAGATTTGCCCTCACAGTAACCCCATGCTGGAAATACCCA | |
| | | ACTCCAGTAGCACTGAAATAACATAATAATATAGGAAGAATAC | |
| | | AAGGACTATAGAAATACAGATTAGTGTTTGAACCCTTGCTTAC | |
| | | CAGCTACTACTAATATGATTGTGGATGAGGTAGCTTCTTACTTA | |
| | | TTAACGGGGATACTAATAGAGGTGGTTCCTATGATTTTTTATTT | |
| | | AATAGTGATACCAAAGCAATACATACTCAGTAGAAACCTACTT | |
| | | CAAGTTCCCATAAAATCATCTGCTTTTCACTTTCAGTACAGTAT | |
| | | TTAATAACTTAAATGAGATATTTCACACTTCAGTAGTAAATACA | |
| | | CTTTTTGTTAGATAATTTTGTCCAACTGTATGCTAATGTAAGTG | |
| | | TTCTGAGCATGTTTAAGGCAGGTTAGGTTAAGCTATGATGTTTG | |
| | | GTGGGTTAGGTGTATTAAATGCATTTCTGATTTTGGATATTTTC | |
| | | AGTGTACAATGGGTTTACAGGGATGTAACCCCATCATAAGTGA | |
| | | AGGAGCACCTGTACTTACTTCATTAAAATGCTGAAACAGTAAA | |
| | | TAAGGTAACATTTAATAATATGTTGTGCAGTTCTTGAAATTTAA | |
| | | GTACTCACCAAATATTACTTTTCCTTTTTTTGTTATTTACTTACT | |
| | | TTTCATTCATTTATTAATTCATTTGTGCATTTAGTAAACATTTAT | |
| | | AAATTATTTCCTGTGCCTGACAGCATGCTGGAACAGTGCTAAA | |
| | | GATACAAGTTAATTAAGACACAATCACGACCCCCAAGATTCCT | |
| | | ACTCTTTTCTAAAGATTACAGACAAGCAGACGATGCTATTGTTG | |
| | | AAGAAACATGCTCTGAGAGGCATTTGAAGGAAGTGTAGAGGA | |
| | | TAGAAGATGGACACATAACCCAGGATGGGGAGGAAAAGAGTT | |
| | | AGGGAAGGCTTTTTGACGAAGATACTGTTTACACCGTGTGTTCT | |
| | | TATAAATTCATGGTGGTGGGGATAGAGTTGGAGGAAAAGGCAT | |
| | | GCTCAGTGGCGTGGAGATGGCAGAGAGATTGGGGTGTTCAAGG | |
| | | ATATGCCGGGAATTCAAGGAACGAGAATTCCCATAGACACAGA | |
| | | CACAGCTAGACATAGAGATCTGCAGCTTAGGTTTGGGCTGTGG | |
| | | GTATAGATCCAGGTGGCTTCAACAGACAAAGATCTTTCCTGAG | |
| | | AAAAGGGAAAAGTTTTCAACACAGAAAGACCATCCCATGTTTG | |
| | | GAATGAGGTTTGCAAATAGATTGCTTGAGGAGAGAAGTATGTG | |
| | | ATCAGAAAGCATTCTTTGTCTATTAACTCCTGCCCAGCAAAAGT | |
| | | GAAAGAAAATTCATGGGAGCATGCAAGAACAAAGAGCACAGC | |
| | | AAAGCTGGACAAACACAGCAATCCAGGCAGGGGATTTCCAACT | |
| | | CAACTCTGGTATATAAGCTGCATGCAAAGTCCTTTTTCTGTCTC | |
| | | TGGTTTCTGGCCCCTTGTCTGCAGAG | |
| SB12098 | UBD_1_-_ | TCTCCTTCACCTTCCACCATGAGTAAAAGCTTCTTGAGGCCTCA | 464 |
| | m_25 | CCAGAAGCAGATGCTGGTGCCATACTTCTTGTATAGTTGGAGG | |
| | | AACTATGAATCAATTAAACactegaattcagaAAATTACCCAGCCTCA | |
| | | AGTATTCCTTTATAGTATGCAAAAGAGACTAAAACAGCAAATG | |
| | | AAAAGAGATCTGTCTCTGAAAGAACTTATTGattctagccttacagcctaa | |
| | | AAAAGTCAGATTGACACTGGGTAAGAAGCAAGGAGGTCAGGT | |
| | | CCCAAGATGAAGTCCTGCCTGTGGTCAGCAAAGGGGCACCAAG | |
| | | GTGTCTGGGACAGTCCTGGCCCTGGCTTTGGGCAGGGAGGGAA | |
| | | TTTCCCATAGGAAGGGAAGAGTAAAGAGAGAGAGAGAGGTCA | |
| | | GAGTCCAGGTTTGTTGTTCATATGTTTTCTTGAAAGGGCACTAT | |
| | | TTCCCAGAATCCAGGTCATCTCTGGGTAGGGAATCCCCTGAATT | |
| | | AGTTTTTTCTGAGAGTATATTAGATTTGCCCTCACAGTAACCCC | |
| | | ATGCTGGAAATACCCAACTCCAGTAGCActgtaatatcatccgctctttaCTG | |
| | | AAATAACATAATAATATAGGAAGAATACAAGGACTATAGAAA | |
| | | TACAGATTAGTGTTTGAACCCTTGCTTACCAGCTACTACTAATA | |
| | | TGATTGTGGATGAGGTAGCTTCTTACTTATTAACGGGGATACTA | |
| | | ATAGAGGTGGTTCCTtgatcggccaatatttATGATTTTTTATTTAATAGT | |
| | | GATACCAAAGCAATACATACTCAGTAGAAACCTACTTCAAGTT | |
| | | CCCATAAAATCATCTGCTTTTCACTTTCAGTACAGTATTTAATA | |
| | | ACTTAAATGAGATATTTCACACTTCAGTAGTAAATACACTTTTT | |
| | | GTTAGATAATTTTGTCCAACTGTATGCTAATGTAAGTGTTCTGA | |
| | | GCATGTTTAAGGCAGGTTAGGTTAAGCTATGATGTTTGGTGGG | |
| | | TTAGGTGTATTAAATGCATTTCTGATTTTGGATATTTTCAGTGT | |
| | | ACAATGGGTTTACAGGGATGTAACCCCATCATAAGTGAAGGAG | |
| | | CACCTGTACTTACTTCATTAAAATGCTGAAACAGTAAATAAGG | |
| | | TAACATTTAATAATATGTTGTGCAGTTCTTGAAATTTAAGTACT | |
| | | CACCAAATATTACTTTTCCTTTTTTTGTTATTTACTTACTTTTCA | |
| | | TTCATTTATTAATTCATTTGTGCATTTAGTAAACATTTATAAATT | |
| | | ATTTCCTGTGCCTGACAGCATGCTGGAACAGTGCTAAAGATAC | |
| | | AAGTTAATTAAGACACAATCACGACCCCCAAGATTCCTACTCT | |
| | | TTTCTAAAGATTACAGACAAGCAGACGATGCTATTGTTGAAGA | |
| | | AACATGCTCTGAGAGGCATTTGAAGGAAGTGTAGAGGATAGA | |
| | | AGATGGACACATAACCCAGGATGGGGAGGAAAAGAGTTAGGG | |
| | | AAGGCTTTTTGACGAAGATACTGTTTACACCGTGTGTTCTTATA | |
| | | AATTCATGGTGGTGGGGATAGAGTTGGAGGAAAAGGCATGCTC | |
| | | AGTGGCGTGGAGATGGCAGAGAGATTGGGGTGTTCAAGGATAT | |
| | | GCCGGGAATTCAAGGAACGAGAATTCCCATAGACACAGACAC | |
| | | AGCTAGACATAGAGATCTGCAGCTTAGGTTTGGGCTGTGGGTA | |
| | | TAGATCCAGGTGGCTTCAACAGACAAAGATCTTTCCTGAGAAA | |
| | | AGGGAAAAGTTTTCAACACAGAAAGACCATCCCATGTTTGGAA | |
| | | TGAGGTTTGCAAATAGATTGCTTGAGGAGAGAAGTATGTGATC | |
| | | AGAAAGCATTCTTTGTCTATTAACTCCTGCCCAGCAAAAGTGA | |
| | | AAGAAAATTCATGGGAGCATGCAAGAACAAAGAGCACAGCAA | |
| | | AGCTGGACAAACACAGCAATCCAGGCAGGGGATTTCCAACTCA | |
| | | ACTCTGGTATATAAGCTGCATGCAAAGTCCTTTTTCTGTCTCTG | |
| | | GTTTCTGGCCCCTTGTCTGCAGAG | |
| SB12099 | UBD_1_-_ | TCTCCTTCACCTTCCACCATGAGTAAAAGCTTCTTGAGGCCTCA | 465 |
| | m_28 | CCAGAAGCAGATGCTGGTGCCATACTTCTTGTATAGTTGGAGG | |
| | | AACTATGAATCAATTAAACAAATTACCCAGCCTCAAGTATTCC | |
| | | TTTATAGTATGCAAAAGAGACTAAAACAGCAAATGAAAAGAG | |
| | | ATCTGTCTCTGAAAGAACTTATTGAAAAGTCAGATTGACACTG | |
| | | GGTAAGAAGCAAGGAGGTCAGGTCCCAAGATGAAGTCCTGCCT | |
| | | GTGGTCAGCAAAGGGGCACCAAGGTGTCTGGGACAGTCCTGGC | |
| | | CCTGGCTTTGGGCAGGGAGGGAATTTCCCATAGGAAGGGAAGA | |
| | | GTAAAGAGAGAGAGAGAGGTCAGAGTCCAGGTTTGTTGTTCAT | |
| | | ATGTTTTCTTGAAAGGGCACTATTTCCCAGAATCCAGGTCATCT | |
| | | CTGGGTAGGGAATCCCCTGAATTAGTTTTTTCTGAGAGTATATT | |
| | | AGATTTGCCCTCACAGTAACCCCATGCTGGAAATACCCAACTC | |
| | | CAGTAGCACTGAAATAACATAATAATATAGGAAGAATACAAG | |
| | | GACTATAGAAATACAGATTAGTGTTTGAACCCTTGCTTACCAG | |
| | | CTACTACTAATATGATTGTGGATGAGGTAGCTTCTTACTTATTA | |
| | | ACGGGGATACTAATAGAGGTGGTTCCTATGATTTTTTATTTAAT | |
| | | AGTGATACCAAAGCAATACATACTCAGTAGAAACCTACTTCAA | |
| | | GTTCCCATAAAATCATCTGCTTTTCACTTTCAGTACAGTATTTA | |
| | | ATAACTTAAATGAGATATTTCACACTTCAGTAGTAAATACACTT | |
| | | TTTGTTAGATAATTTTGTCCAACTGTATGCTAATGTAAGTGTTC | |
| | | TGAGCATGTTTAAGGCAGGTTAGGTTAAGCTATGATGTTTGGT | |
| | | GGGTTAGGTGTATTAAATGCATTTCTGATTTTGGATATTTTCAG | |
| | | TGTACAATGGGTTTACAGGGATGTAACCCCATCATAAGTGAAG | |
| | | GAGCACCTGTACTTACTTCATTAAAATGCTGAAACAGTAAATA | |
| | | AGGTAACATTTAATAATATGTTGTGCAGTTCTTGAAATTTAAGT | |
| | | ACTCACCAAATATTACTTTTCCTTTTTTTGTTATTTACTTACTTT | |
| | | TCATTCATTTATTAATTCATTTGTGCATTTAGTAAACATTTATAA | |
| | | ATTATTTCCTGTGCCTGACAGCATGCTGGAACAGTGCTAAAGA | |
| | | TACAAGTTAATTAAGACACAATCACGACCCCCAAGATTCCTAC | |
| | | TCTTTTCTAAAGATTACAGACAAGCAGACGATGCTATTGTTGA | |
| | | AGAAACATGCTCTGAGAGGCATTTGAAGGAAGTGTAGAGGAT | |
| | | AGAAGATGGACACATAACCCAGGATGGGGAGGAAAAGAGTTA | |
| | | GGGAAGGCTTTTTGACGAAGATACTGTTTACACCGTGTGTTCTT | |
| | | ATAAATTCATGGTGGTGGGGATAGAGTTGGAGGAAAAGGCAT | |
| | | GCTCAGTGGCGTGGAGATGGCAGAGAGATTGGGGTGTTCAAGG | |
| | | ATATGCCGGGAATTCAAGGAACGAGAATTCCCATAGACACAGA | |
| | | CACAGCTAGACATAGAGATCTGCAGCTTAGGTTTGGGCTGTGG | |
| | | GTATAGATCCAGGTGGCTTCAACAGACAAAGATCTTTCCTGAG | |
| | | AAAAGGGAAAAGTTTTCAACACAGAAAGACCATCCCATGTTTG | |
| | | GAATGAGGTTTGCAAATAGATTGCTTGAGGAGAGAAGTATGTG | |
| | | ATCAGAAAGCATTCTTTGTCTATTAACTCCTGCCCAGCAAAAGT | |
| | | GAAAGAAAATTCATGGGAGCATGCAAGAACAAAGAGCACAGC | |
| | | AAAGCTGGACAAACACAGCAATCCAGGCAGGGGATTTCCAACT | |
| | | CAACTCTGGTATATAAGCTGCATGCAAAGTCCTTTTTCTGTCTC | |
| | | TGGTTTCTGGCCCCTTGTCTGCAGAG | |
| SB12093 | UBD_1_-_ | GGTTTGTTGTTCATATGTTTTCTTGAAAGGGCACTATTTCCCAG | 494 |
| | m_20 | AATCCAGGTCATCTCTGGGTAGGGAATCCCCTGAATTAGTTTTT | |
| | | TCTGAGAGTATATTAGATTTGCCCTCACAGTAACCCCATGCTGG | |
| | | AAATACCCAACTCCAGTAGCAACTAGTTCTAGAGGGTATATAA | |
| | | TGGGGGCCA | |
| SB12094 | UBD_1_-_ | GGTTTGTTGTTCATATGTTTTCTTGAAAGGGCACTATTTCCCAG | 495 |
| | m_21 | AATCCAGGTCATCTCTGGGTAGGGAATCCCCTGAATTAGTTTTT | |
| | | TCTGAGAGTATATTAGATTTGCCCTCACAGTAACCCCATGCTGG | |
| | | AAATACCCAACTCCAGTAGCAGGTTTGTTGTTCATATGTTTTCT | |
| | | TGAAAGGGCACTATTTCCCAGAATCCAGGTCATCTCTGGGTAG | |
| | | GGAATCCCCTGAATTAGTTTTTTCTGAGAGTATATTAGATTTGC | |
| | | CCTCACAGTAACCCCATGCTGGAAATACCCAACTCCAGTAGCA | |
| | | GGTTTGTTGTTCATATGTTTTCTTGAAAGGGCACTATTTCCCAG | |
| | | AATCCAGGTCATCTCTGGGTAGGGAATCCCCTGAATTAGTTTTT | |
| | | TCTGAGAGTATATTAGATTTGCCCTCACAGTAACCCCATGCTGG | |
| | | AAATACCCAACTCCAGTAGCAACTAGTTCTAGAGGGTATATAA | |
| | | TGGGGGCCA | |

TABLE 2
DNA sequences of the wildtype ablation motifs and the second nucleotide motifs for
promoter ablation variants with substitutions described in Table 1 above. For clarity:
SB07097- SB07121 are promoter ablation variants of SB05116; SB09386-SB09405 are
promoter ablation variants of SB05125; SB09407-SB09425 are promoter ablation
variants of SB05132.

| | | | | Second Nucleotide |
|---|---|---|---|---|
| Promoter ID | Wildtype Sequence | NT start pos. | NT end pos. | Motif |
| SB07097 | TGTAATCCCA | 63 | 73 | CTTACCTACT |
| | SEQ ID NO: 170 | | | SEQ ID NO: 171 |
| SB07098 | GGGAAGCGGAG | 80 | 102 | AATTCAGACGAC |
| | GCAAGCAGATC | | | AAACCATTCT |
| | SEQ ID NO: 172 | | | SEQ ID NO: 173 |
| SB07099 | GCCTAGCCAACA | 141 | 162 | TTCTAAGTCCAA |
| | AGGTGAAAC | | | TTCACGACA |
| | SEQ ID NO: 174 | | | SEQ ID NO: 175 |
| SB07100 | TGTAATCCCA | 212 | 222 | GTTGAAGCTT |
| | SEQ ID NO: 176 | | | SEQ ID NO: 177 |
| SB07101 | AGGAGGCTGAG | 229 | 251 | GAGTCGTCAGAC |
| | GCAGGAGAATC | | | TCAATTATTA |
| | SEQ ID NO: 178 | | | SEQ ID NO: 179 |
| SB07102 | GCCTGGGTGACA | 307 | 361 | AATTGGAACCAC |
| | GAGTGAGACTCT | | | GTATCTACTGCA |
| | GTCTCAAAAAA | | | TTGTAACTACAA |
| | AAAAAAAAAAA | | | CAGCTCGAGGTA |
| | AAGGACAA | | | TTAGAT |
| | SEQ ID NO: 180 | | | SEQ ID NO: 181 |
| SB07103 | AGAACAATGGC | 365 | 376 | GGTGAATTTTC |
| | SEQ ID NO: 182 | | | SEQ ID NO: 183 |
| SB07104 | GCTCTGTCTGCA | 559 | 571 | TACTCATCACTA |
| | SEQ ID NO: 184 | | | SEQ ID NO: 185 |
| SB07105 | AAGTGGCAGAC | 617 | 633 | TGCTAGTTGTCC |
| | AGGCG | | | AATA |
| | SEQ ID NO: 186 | | | SEQ ID NO: 187 |
| SB07106 | CATCAATGGTAT | 782 | 799 | CGTGTGTCATAT |
| | TGTTT | | | AGAAT |
| | SEQ ID NO: 188 | | | SEQ ID NO: 189 |
| SB07107 | CATAGTAAACA | 852 | 871 | AACAGTCTAAGT |
| | ATCAAAAA | | | CCTCAAA |
| | SEQ ID NO: 190 | | | SEQ ID NO: 191 |
| SB07108 | TATGATTAATGA | 886 | 920 | ACTCTACGGAAG |
| | ATGGAAAGAAG | | | TAGCTTGTTTAA |
| | CTAAGTCAGAA | | | |
| | SEQ ID NO: 192 | | | AACCTATAGT |
| | | | | SEQ ID NO: 193 |
| SB07109 | ATGTTCCCCCTC | 933 | 959 | GTTCTACTAGTA |
| | CCTCTCTCCATC | | | CAAAGGTACCA |
| | CA | | | GTA |
| | SEQ ID NO: 194 | | | SEQ ID NO: 195 |
| SB07110 | TCACATTTAGTT | 1002 | 1028 | TGAGTAAACTAA |
| | TCTCTTTCAAGG | | | CTTTCAACCGCT |
| | CC | | | CT |
| | SEQ ID NO: 196 | | | SEQ ID NO: 197 |
| SB07111 | CTTCTGACCTTT | 1032 | 1045 | TCGTTACCATCT |
| | C | | | T |
| | SEQ ID NO: 198 | | | SEQ ID NO: 199 |
| SB07112 | CTTGTCCCCTCC | 1064 | 1087 | AAACACCGTTTT |
| | CCCATCGGCCG | | | GCTGTAATATC |
| | SEQ ID NO: 200 | | | SEQ ID NO: 201 |
| SB07113 | AGAAAAGGGGG | 1169 | 1192 | CGCGTAGAACTT |
| | AAAATGAAGGG | | | CGTAACATTAA |
| | G | | | SEQ ID NO: 203 |
| | SEQ ID NO: 202 | | | |
| SB07114 | CTCCAGCTGTTC | 1212 | 1232 | AGATAACGCCGT |
| | CTTTGACC | | | CATTGTAT |
| | SEQ ID NO: 204 | | | SEQ ID NO: 205 |
| SB07115 | AAATAGAGGAA | 1257 | 1275 | TAACATCGTTCT |
| | GGGGAGG | | | CAGCTA |
| | SEQ ID NO: 206 | | | SEQ ID NO: 207 |
| SB07116 | GGGGTCCTGGGC | 1310 | 1333 | ATATACAGTGTT |
| | CTCCTCCCAAG | | | CAGCGTGTTAC |
| | SEQ ID NO: 208 | | | SEQ ID NO: 209 |
| SB07117 | ACATAGTGGGC | 1381 | 1434 | GACGTCTGTTAG |
| | AGGCACAGTGA | | | TAGTATTACCCG |
| | TGACCTTGGAGG | | | TGTATTTCGGTC |
| | CCACCCCAGGAT | | | TTCGAGCAATTA |
| | CTCATGG | | | CTTTA |
| | SEQ ID NO: 210 | | | SEQ ID NO: 211 |
| SB07118 | GGAAAGCAGAA | 1698 | 1753 | GTGCATAAAAA |
| | GGAAAGGGCAA | | | GAAATTCACCAC |
| | CCGCAGGGGGA | | | GAGTACCTATCT |
| | AGAAGGGGGGC | | | TGGTCTCGTTTG |
| | AGGGTGCGATT | | | TTGCACTA |
| | SEQ ID NO: 212 | | | SEQ ID NO: 213 |
| SB07119 | CACAGAATGGG | 1783 | 1826 | AAAAACTACCA |
| | ACATGAAGGGG | | | ACCAGTTATCAT |
| | AATTTCAGGCAG | | | TTCTCTGTGTAA |
| | AGAAAGTGA | | | TATCTGAA |
| | SEQ ID NO: 214 | | | SEQ ID NO: 215 |
| SB07120 | CCCAGTCGCCCT | 1909 | 1927 | CGCAGAATATCG |
| | GCAGCC | | | ATATCT |
| | SEQ ID NO: 216 | | | SEQ ID NO: 217 |
| SB07121 | ACCCAGACAGCT | 1946 | 1961 | CGAATAGCACCT |
| | GGC | | | ATA |
| | SEQ ID NO: 218 | | | SEQ ID NO: 219 |
| SB09386 | GTTAAGTGGCT | 133 | 144 | CTTACCTACTA |
| | SEQ ID NO: 220 | | | SEQ ID NO: 221 |
| SB09387 | ATTGTTTTTTTTT | 200 | 217 | TAATTCGTCCGA |
| | TTTT | | | TAGAT |
| | SEQ ID NO: 222 | | | SEQ ID NO: 223 |
| SB09388 | AGTCTCACTCTG | 225 | 247 | AATTCAGACGAC |
| | CTGCCCAGGC | | | AAACCATTCT |
| | SEQ ID NO: 224 | | | SEQ ID NO: 225 |
| SB09389 | AATTCTCCTGCC | 303 | 325 | TTCTAAGTCCAA |
| | TCAGCCTCCC | | | TTCACGACAA |
| | SEQ ID NO: 226 | | | SEQ ID NO: 227 |
| SB09390 | TGGGATTACA | 332 | 342 | GTTGAAGCTT |
| | SEQ ID NO: 228 | | | SEQ ID NO: 229 |
| SB09391 | GGTTTCACCATG | 391 | 413 | AGTCGTCAGACT |
| | TTGCCCAGGC | | | CAATTATTAC |
| | SEQ ID NO: 230 | | | SEQ ID NO: 231 |
| SB09392 | CTCCTGACCTCA | 423 | 460 | TCCCTAGCGATC |
| | AGTGATCCGCCC | | | GAAGTTGATAA |
| | TCCTCAGCTTCC | | | AACCTAAGTTTT |
| | C | | | GT |
| | SEQ ID NO: 232 | | | SEQ ID NO: 233 |
| SB09393 | TGGGATTACA | 332 | 342 | GCCTTCATAA |
| | SEQ ID NO: 234 | | | SEQ ID NO: 235 |
| SB09394 | TTTTATTTGTAG | 693 | 717 | TCTCGCTAATAG |
| | TGTTGTTTTCTA | | | GAGTAAGATAC |
| | SEQ ID NO: 236 | | | A |
| | | | | SEQ ID NO: 237 |
| SB09395 | TGATTTTTTGTTT | 738 | 761 | TTCTGCTGCAAG |
| | GTTTTCCTTG | | | ACCTATACTAT |
| | SEQ ID NO: 238 | | | SEQ ID NO: 239 |
| SB09396 | ATTTTGGTTTCA | 838 | 861 | CCACATTGCTAT |
| | GTTTTCCTTAC | | | AGTGCTGTATA |
| | SEQ ID NO: 240 | | | SEQ ID NO: 241 |
| SB09397 | ATTTTATCCTTTT | 1229 | 1246 | TGCGTACCAGAA |
| | CTCC | | | TATTT |
| | SEQ ID NO: 242 | | | SEQ ID NO: 243 |
| SB09398 | GTTGCCTCATCA | 1286 | 1309 | TGGTCACTATCA |
| | GCCCCCCGCAG | | | CGTATATACCA |
| | SEQ ID NO: 244 | | | SEQ ID NO: 245 |
| SB09399 | AAAATAAGGAA | 1413 | 1431 | CGAGTTCGATAA |
| | GTGGAGA | | | TACACT |
| | SEQ ID NO: 246 | | | SEQ ID NO: 247 |
| SB09400 | TGCTTCTATTTTT | 1456 | 1473 | AATACTGGTGCT |
| | TTCC | | | TCAAT |
| | SEQ ID NO: 248 | | | SEQ ID NO: 249 |
| SB09401 | TGAAACATGAC | 1530 | 1544 | CCGATAGAAAG |
| | ACT | | | AAT |
| | SEQ ID NO: 250 | | | SEQ ID NO: 251 |
| SB09402 | AAGCCAATGAG | 1577 | 1590 | TGTCTGTATAAA |
| | AA | | | G |
| | SEQ ID NO: 252 | | | SEQ ID NO: 253 |
| SB09403 | TTTGTGGTTTTA | 1816 | 1836 | TGTTAAGCATAC |
| | TTGGTTTT | | | TAAACTGT |
| | SEQ ID NO: 254 | | | SEQ ID NO: 255 |
| SB09404 | AAACTAGAAAA | 1852 | 1872 | TTTCGAGCGACG |
| | TGAAACCAT | | | CTTAATAT |
| | SEQ ID NO: 256 | | | SEQ ID NO: 257 |
| SB09405 | AAAGTGGAAGT | 1876 | 1896 | TAGATAGTACGG |
| | AATTTCTCA | | | GTTCCATA |
| | SEQ ID NO: 258 | | | SEQ ID NO: 259 |
| SB09407 | CACCAGAAGCA | 43 | 60 | CTTACCTACTAG |
| | GATGCT | | | GTTAA |
| | SEQ ID NO: 260 | | | SEQ ID NO: 261 |
| SB09408 | TTTTTTTCCTTAT | 107 | 120 | ACTCGAATTCAG |
| | | | | A |
| | SEQ ID NO: 262 | | | SEQ ID NO: 263 |
| SB09409 | TGACATAGAGA | 210 | 230 | ATTCTAGCCTTA |
| | GAGACAGAA | | | CAGCCTAA |
| | SEQ ID NO: 264 | | | SEQ ID NO: 265 |
| SB09410 | GGCAGGGAGGG | 345 | 407 | ACTCTACGGAAG |
| | AATTTCCCATAG | | | TAGCTTGTTTAA |
| | GAAGGGAAGAG | | | AACCTATAGTCT |
| | TAAAGAGAGAG | | | CTTCGGAGTCGT |
| | AGAGAGGTCAG | | | TCTACTAGTACA |
| | AGTCCA | | | AA |
| | SEQ ID NO: 266 | | | SEQ ID NO: 267 |
| SB09411 | TCTTGAAAGGGC | 427 | 457 | TGAGTAAACTAA |
| | ACTATTTCCCAG | | | CTTTCAACCGCT |
| | AATCCA | | | CTTCGT |
| | SEQ ID NO: 268 | | | SEQ ID NO: 269 |
| SB09412 | GGTAGGGAATC | 468 | 484 | CTTAAACACCGT |
| | CCCTG | | | TTTG |
| | SEQ ID NO: 270 | | | SEQ ID NO: 271 |
| SB09413 | CTTTTCTCCCTG | 560 | 582 | CTGTAATATCAT |
| | CCTTTTCCCA | | | CCGCTCTTTA |
| | SEQ ID NO: 272 | | | SEQ ID NO: 273 |
| SB09414 | TACAATGATTCC | 730 | 746 | TGATCGGCCAAT |
| | ATTT | | | ATTT |
| | SEQ ID NO: 391 | | | SEQ ID NO: 274 |
| SB09415 | CCCATAAAATC | 809 | 820 | TAGAACTTCGT |
| | SEQ ID NO: 275 | | | SEQ ID NO: 276 |
| SB09416 | TTTCACTTTC | 827 | 837 | AACATTAAGT |
| | SEQ ID NO: 277 | | | SEQ ID NO: 278 |
| SB09417 | AATGAGATATTT | 858 | 878 | TAGATAACGCCG |
| | CACACTTC | | | TCATTGTA |
| | SEQ ID NO: 279 | | | SEQ ID NO: 280 |
| SB09418 | AAGTTAATTAA | 1291 | 1302 | TTTCTCTAACG |
| | SEQ ID NO: 281 | | | SEQ ID NO: 282 |
| SB09419 | AGATTCCTACTC | 1321 | 1341 | CTAACATCGTTC |
| | TTTTCTAA | | | TCAGCTAA |
| | SEQ ID NO: 283 | | | SEQ ID NO: 284 |
| SB09420 | CAGGATGGGGA | 1435 | 1463 | TATACAGTGTTC |
| | GGAAAAGAGTT | | | AGCGTGTTACTT |
| | AGGGAA | | | GTGA |
| | SEQ ID NO: 285 | | | SEQ ID NO: 286 |
| SB09421 | GGAGGAAAAGG | 1530 | 1541 | CGTACAAGTAT |
| | SEQ ID NO: 287 | | | SEQ ID NO: 288 |
| SB09422 | TTTCCTGAGAA | 1707 | 1718 | AGTCTCTGAAT |
| | SEQ ID NO: 289 | | | SEQ ID NO: 290 |
| SB09423 | CCCAGCAAAAG | 1834 | 1863 | CCCTATATAATA |
| | TGAAAGAAAAT | | | CCCGCTAGCATA |
| | TCATGGG | | | CAAAT |
| | SEQ ID NO: 291 | | | SEQ ID NO: 292 |
| SB09424 | AAGAACAAAGA | 1870 | 1882 | GTTGCTCATATA |
| | G | | | SEQ ID NO: 294 |
| | SEQ ID NO: 293 | | | |
| SB09425 | GGCAGGGGATTT | 1913 | 1929 | ACGTCTGTTAGT |
| | CCAA | | | AGTA |
| | SEQ ID NO: 295 | | | SEQ ID NO: 296 |

In some embodiments, a promoter of the present disclosure may comprise one or more transcriptional activating elements. In some embodiments, a transcriptional activating element is or comprises a nucleotide sequence as shown in Table 2. In some embodiments, a promoter of the present disclosure comprises at least one transcriptional activating element. In some embodiments, a promoter of the present disclosure comprises at least two transcriptional activating elements. In some embodiments, a promoter of the present disclosure comprises at least three transcriptional activating elements. In some embodiments, a promoter of the present disclosure comprises at least four transcriptional activating elements. In some embodiments, a promoter of the present disclosure comprises at least five transcriptional activating elements. In some embodiments, a promoter of the present disclosure comprises at least six transcriptional activating elements. In some embodiments, a promoter of the present disclosure comprises at least seven transcriptional activating elements. In some embodiments, a promoter of the present disclosure comprises at least eight transcriptional activating elements. In some embodiments, a promoter of the present disclosure comprises at least nine transcriptional activating elements. In some embodiments, a promoter of the present disclosure comprises at least ten transcriptional activating elements. In some embodiments, two or more transcriptional activating elements are contiguous. In some embodiments, two or more transcriptional activating elements are non-contiguous.

In some embodiments, a promoter of the present disclosure does not comprise one or more repressive elements. In some embodiments, a repressive element is or comprises a nucleotide sequence as shown in Table 2. In some embodiments, a promoter of the present disclosure does not comprise at least one repressive element. In some embodiments, a promoter of the present disclosure does not comprise at least two repressive elements. In some embodiments, a promoter of the present disclosure does not comprise at least three repressive elements. In some embodiments, a promoter of the present disclosure does not comprise at least four repressive elements. In some embodiments, a promoter of the present disclosure does not comprise at least five repressive elements. In some embodiments, a promoter of the present disclosure does not comprise at least six repressive elements. In some embodiments, a promoter of the present disclosure does not comprise at least seven repressive elements. In some embodiments, a promoter of the present disclosure does not comprise at least eight repressive elements. In some embodiments, a promoter of the present disclosure does not comprise at least nine repressive elements. In some embodiments, a promoter of the present disclosure does not comprise at least ten repressive elements. In some embodiments, two or more repressive elements are contiguous. In some embodiments, two or more repressive elements are non-contiguous.

In some embodiments, a promoter of the present disclosure may comprise an ablation of at least two nucleotide motifs relative to a promoter from Table 1. In some embodiments, a promoter of the present disclosure may comprise an ablation of at least three nucleotide motifs relative to a promoter from Table 1. In some embodiments, a promoter of the present disclosure may comprise an ablation of at least four nucleotide motifs relative to a promoter from Table 1. In some embodiments, a promoter of the present disclosure may comprise an ablation of at least five nucleotide motifs relative to a promoter from Table 1. In some embodiments, a promoter of the present disclosure may comprise an ablation of at least six nucleotide motifs relative to a promoter from Table 1. In some embodiments, a promoter of the present disclosure may comprise an ablation of at least seven nucleotide motifs relative to a promoter from Table 1. In some embodiments, a promoter of the present disclosure may comprise an ablation of at least eight nucleotide motifs relative to a promoter from Table 1. In some embodiments, a promoter of the present disclosure may comprise an ablation of at least nine nucleotide motifs relative to a promoter from Table 1. In some embodiments, a promoter of the present disclosure may comprise an ablation of ten or more nucleotide motifs relative to a promoter from Table 1. In some embodiments, the nucleotide motifs are selected from Table 2. In certain embodiments, a promoter of the present disclosure comprises ablation and substitution of a nucleotide motif as presented in Table 2.

In some embodiments, an engineered promoter of the present disclosure may comprise an ablation of two nucleotide motifs relative to SEQ ID NO:132. In some embodiments, an engineered promoter of the present disclosure may comprise an ablation of three nucleotide motifs relative to SEQ ID NO:132. In some embodiments, an engineered promoter of the present disclosure may comprise an ablation of four nucleotide motifs relative to SEQ ID NO:132. In some embodiments, an engineered promoter of the present disclosure may comprise an ablation of five nucleotide motifs relative to SEQ ID NO:132. In some embodiments, an engineered promoter of the present disclosure may comprise an ablation of six nucleotide motifs relative to SEQ ID NO:132. In some embodiments, an engineered promoter of the present disclosure may comprise an ablation of seven nucleotide motifs relative to SEQ ID NO:132. In some embodiments, an engineered promoter of the present disclosure may comprise an ablation of eight nucleotide motifs relative to SEQ ID NO:132. In some embodiments, a engineered promoter of the present disclosure may comprise an ablation of nine nucleotide motifs relative to SEQ ID NO:132. In some embodiments, a engineered promoter of the present disclosure may comprise an ablation of ten nucleotide motifs relative to SEQ ID NO:132. In some embodiments, the nucleotide motifs are selected from Table 2.

In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:143. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:144. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:145. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:146. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:147. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:148. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:149. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:150. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:151. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:152. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:153. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:154. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:155. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:156. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:157. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:158. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:159. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:160. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:161. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:162. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:163. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:1. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:2. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:3.

In some embodiments, an engineered promoter ablation variant of the present disclosure comprises the nucleotide sequence of SEQ ID NO:143. In some embodiments, an engineered promoter ablation variant of the present disclosure comprises the nucleotide sequence of SEQ ID NO:144. In some embodiments, an engineered promoter ablation variant of the present disclosure comprises the nucleotide sequence of SEQ ID NO:145. In some embodiments, an engineered promoter ablation variant of the present disclosure comprises the nucleotide sequence of SEQ ID NO:146. In some embodiments, an engineered promoter ablation variant of the present disclosure comprises the nucleotide sequence of SEQ ID NO:147. In some embodiments, an engineered promoter ablation variant of the present disclosure comprises the nucleotide sequence of SEQ ID NO:148. In some embodiments, an engineered promoter ablation variant of the present disclosure comprises the nucleotide sequence of SEQ ID NO:149. In some embodiments, an engineered promoter ablation variant of the present disclosure comprises the nucleotide sequence of SEQ ID NO:150. In some embodiments, an engineered promoter ablation variant of the present disclosure comprises the nucleotide sequence of SEQ ID NO:151. In some embodiments, an engineered promoter ablation variant of the present disclosure comprises the nucleotide sequence of SEQ ID NO:152. In some embodiments, an engineered promoter ablation variant of the present disclosure comprises the nucleotide sequence of SEQ ID NO:153. In some embodiments, an engineered promoter ablation variant of the present disclosure comprises the nucleotide sequence of SEQ ID NO:154. In some embodiments, an engineered promoter ablation variant of the present disclosure comprises the nucleotide sequence of SEQ ID NO:155. In some embodiments, an engineered promoter ablation variant of the present disclosure comprises the nucleotide sequence of SEQ ID NO:156. In some embodiments, an engineered promoter ablation variant of the present disclosure comprises the nucleotide sequence of SEQ ID NO:157. In some embodiments, an engineered promoter ablation variant of the present disclosure comprises the nucleotide sequence of SEQ ID NO:158. In some embodiments, an engineered promoter ablation variant of the present disclosure comprises the nucleotide sequence of SEQ ID NO:159. In some embodiments, an engineered promoter ablation variant of the present disclosure comprises the nucleotide sequence of SEQ ID NO:160. In some embodiments, an engineered promoter ablation variant of the present disclosure comprises the nucleotide sequence of SEQ ID NO:161. In some embodiments, an engineered promoter ablation variant of the present disclosure comprises the nucleotide sequence of SEQ ID NO:162. In some embodiments, an engineered promoter ablation variant of the present disclosure comprises the nucleotide sequence of SEQ ID NO:163. In some embodiments, an engineered promoter ablation variant of the present disclosure comprises the nucleotide sequence of SEQ ID NO:1. In some embodiments, an engineered promoter ablation variant of the present disclosure comprises the nucleotide sequence of SEQ ID NO:2. In some embodiments, an engineered promoter ablation variant of the present disclosure comprises the nucleotide sequence of SEQ ID NO:3.

In some embodiments, an engineered promoter of the present disclosure may comprise an ablation of two nucleotide motifs relative to SEQ ID NO:136. In some embodiments, an engineered promoter of the present disclosure may comprise an ablation of three nucleotide motifs relative to SEQ ID NO: 136. In some embodiments, an engineered promoter of the present disclosure may comprise an ablation of four nucleotide motifs relative to SEQ ID NO: 136. In some embodiments, an engineered promoter of the present disclosure may comprise an ablation of five nucleotide motifs relative to SEQ ID NO: 136. In some embodiments, an engineered promoter of the present disclosure may comprise an ablation of six nucleotide motifs relative to SEQ ID NO: 136. In some embodiments, an engineered promoter of the present disclosure may comprise an ablation of seven nucleotide motifs relative to SEQ ID NO: 136. In some embodiments, an engineered promoter of the present disclosure may comprise an ablation of eight nucleotide motifs relative to SEQ ID NO: 136. In some embodiments, an engineered promoter of the present disclosure may comprise an ablation of nine nucleotide motifs relative to SEQ ID NO: 136. In some embodiments, an engineered promoter of the present disclosure may comprise an ablation of ten nucleotide motifs relative to SEQ ID NO: 136. In some embodiments, the nucleotide motifs are selected from Table 2.

In some embodiments, an engineered promoter of the present disclosure may comprise an ablation of two nucleotide motifs relative to SEQ ID NO:392. In some embodiments, an engineered promoter of the present disclosure may comprise an ablation of three nucleotide motifs relative to SEQ ID NO: 392. In some embodiments, an engineered promoter of the present disclosure may comprise an ablation of four nucleotide motifs relative to SEQ ID NO: 392. In some embodiments, an engineered promoter of the present disclosure may comprise an ablation of five nucleotide motifs relative to SEQ ID NO: 392. In some embodiments, an engineered promoter of the present disclosure may comprise an ablation of six nucleotide motifs relative to SEQ ID NO: 392. In some embodiments, an engineered promoter of the present disclosure may comprise an ablation of seven nucleotide motifs relative to SEQ ID NO: 392. In some embodiments, an engineered promoter of the present disclosure may comprise an ablation of eight nucleotide motifs relative to SEQ ID NO: 392. In some embodiments, an engineered promoter of the present disclosure may comprise an ablation of nine nucleotide motifs relative to SEQ ID NO: 392. In some embodiments, an engineered promoter of the present disclosure may comprise an ablation of ten nucleotide motifs relative to SEQ ID NO: 392. In some embodiments, the nucleotide motifs are selected from Table 2.

In some embodiments, an engineered promoter of the present disclosure may comprise an ablation of two nucleotide motifs relative to SEQ ID NO:393. In some embodiments, an engineered promoter of the present disclosure may comprise an ablation of three nucleotide motifs relative to SEQ ID NO: 393. In some embodiments, an engineered promoter of the present disclosure may comprise an ablation of four nucleotide motifs relative to SEQ ID NO: 393. In some embodiments, an engineered promoter of the present disclosure may comprise an ablation of five nucleotide motifs relative to SEQ ID NO: 393. In some embodiments, an engineered promoter of the present disclosure may comprise an ablation of six nucleotide motifs relative to SEQ ID NO: 393. In some embodiments, an engineered promoter of the present disclosure may comprise an ablation of seven nucleotide motifs relative to SEQ ID NO: 393. In some embodiments, an engineered promoter of the present disclosure may comprise an ablation of eight nucleotide motifs relative to SEQ ID NO: 393. In some embodiments, an engineered promoter of the present disclosure may comprise an ablation of nine nucleotide motifs relative to SEQ ID NO: 393. In some embodiments, an engineered promoter of the present disclosure may comprise an ablation of ten nucleotide motifs relative to SEQ ID NO: 393. In some embodiments, the nucleotide motifs are selected from Table 2.

In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:4. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:5. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:6. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:7. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:8. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:9. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:10. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:11. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:12. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:13. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:14. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:15. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:16. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:17. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:18. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:19. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:20. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:21. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:22. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:23. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:24.

In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:456. In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:457. In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:458.

In some embodiments, an engineered promoter ablation variant of the present disclosure comprises the nucleotide sequence of SEQ ID NO:4. In some embodiments, an engineered promoter ablation variant of the present disclosure comprises the nucleotide sequence of SEQ ID NO:5. In some embodiments, an engineered promoter ablation variant of the present disclosure comprises the nucleotide sequence of SEQ ID NO:6. In some embodiments, an engineered promoter ablation variant of the present disclosure comprises the nucleotide sequence of SEQ ID NO:7. In some embodiments, an engineered promoter ablation variant of the present disclosure comprises the nucleotide sequence of SEQ ID NO:8. In some embodiments, an engineered promoter ablation variant of the present disclosure comprises the nucleotide sequence of SEQ ID NO:9. In some embodiments, an engineered promoter ablation variant of the present disclosure comprises the nucleotide sequence of SEQ ID NO:10. In some embodiments, an engineered promoter ablation variant of the present disclosure comprises the nucleotide sequence of SEQ ID NO:11. In some embodiments, an engineered promoter ablation variant of the present disclosure comprises the nucleotide sequence of SEQ ID NO:12. In some embodiments, an engineered promoter ablation variant of the present disclosure comprises the nucleotide sequence of SEQ ID NO:13. In some embodiments, an engineered promoter ablation variant of the present disclosure comprises the nucleotide sequence of SEQ ID NO:14. In some embodiments, an engineered promoter ablation variant of the present disclosure comprises the nucleotide sequence of SEQ ID NO:15. In some embodiments, an engineered promoter ablation variant of the present disclosure comprises the nucleotide sequence of SEQ ID NO:16. In some embodiments, an engineered promoter ablation variant of the present disclosure comprises the nucleotide sequence of SEQ ID NO:17. In some embodiments, an engineered promoter ablation variant of the present disclosure comprises the nucleotide sequence of SEQ ID NO:18. In some embodiments, an engineered promoter ablation variant of the present disclosure comprises the nucleotide sequence of SEQ ID NO:19. In some embodiments, an engineered promoter ablation variant of the present disclosure comprises the nucleotide sequence of SEQ ID NO:20. In some embodiments, an engineered promoter ablation variant of the present disclosure comprises the nucleotide sequence of SEQ ID NO:21. In some embodiments, an engineered promoter ablation variant of the present disclosure comprises the nucleotide sequence of SEQ ID NO:22. In some embodiments, an engineered promoter ablation variant of the present disclosure comprises the nucleotide sequence of SEQ ID NO:23. In some embodiments, an engineered promoter ablation variant of the present disclosure comprises the nucleotide sequence of SEQ ID NO:24. In some embodiments, an engineered macrophage specific promoter or macrophage specific promoter system of the present disclosure comprises the nucleotide sequence of SEQ ID NO:456. In some embodiments, an engineered macrophage specific promoter or macrophage specific promoter system of the present disclosure comprises the nucleotide sequence of SEQ ID NO:457. In some embodiments, an engineered macrophage specific promoter or macrophage specific promoter system of the present disclosure comprises the nucleotide sequence of SEQ ID NO:458.

In some embodiments, an engineered promoter of the present disclosure may comprise an ablation of two nucleotide motifs relative to SEQ ID NO:137. In some embodiments, an engineered promoter of the present disclosure may comprise an ablation of three nucleotide motifs relative to SEQ ID NO: 137. In some embodiments, an engineered promoter of the present disclosure may comprise an ablation of four nucleotide motifs relative to SEQ ID NO: 137. In some embodiments, an engineered promoter of the present disclosure may comprise an ablation of five nucleotide motifs relative to SEQ ID NO: 137. In some embodiments, an engineered promoter of the present disclosure may comprise an ablation of six nucleotide motifs relative to SEQ ID NO: 137. In some embodiments, an engineered promoter of the present disclosure may comprise an ablation of seven nucleotide motifs relative to SEQ ID NO: 137. In some embodiments, an engineered promoter of the present disclosure may comprise an ablation of eight nucleotide motifs relative to SEQ ID NO: 137. In some embodiments, an engineered promoter of the present disclosure may comprise an ablation of nine nucleotide motifs relative to SEQ ID NO: 137. In some embodiments, an engineered promoter of the present disclosure may comprise an ablation of ten nucleotide motifs relative to SEQ ID NO: 137. In some embodiments, the nucleotide motifs are selected from Table 2.

In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:25. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:26. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:27. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:28. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:29. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:30. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:81. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:82. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:88. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:89. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:90. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:91. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:92. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:96. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:97. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:119. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:120. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:121. In some embodiments, an engineered promoter ablation variant of the present disclosure comprises the nucleotide sequence having at least 80%, at least 85%, at least 90%, at least 95%, at least 96%, at least 97%, at least 98%, at least 99%, or 100% sequence identity to SEQ ID NO:122.

In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:459. In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:460. In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:461. In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:462. In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:463. In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:464. In some embodiments, an engineered macrophage specific promoter or macrophage specific promoter system of the present disclosure comprises the nucleotide sequence having at least 80%, at least 85%, at least 90%, at least 95%, at least 96%, at least 97%, at least 98%, at least 99%, or 100% sequence identity to SEQ ID NO:465.

In some embodiments, a promoter ablation variant of the present disclosure does not comprise substitution at the ablation. In some embodiments, a promoter ablation variant of the present disclosure comprises a deletion at the ablation. In some embodiments, an engineered promoter of the present disclosure comprises a nucleotide sequence selected from the group consisting having at least 80%, at least 85%, at least 90%, at least 95%, at least 96%, at least 97%, at least 98%, at least 99%, or 100% sequence identity to SEQ ID NOs: 297-390. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 297. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 298. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 299.

In some embodiments, an engineered promoter ablation variant of the present disclosure comprises the nucleotide sequence of SEQ ID NO:25. In some embodiments, an engineered promoter ablation variant of the present disclosure comprises the nucleotide sequence of SEQ ID NO:26. In some embodiments, an engineered promoter ablation variant of the present disclosure comprises the nucleotide sequence of SEQ ID NO:27. In some embodiments, an engineered promoter ablation variant of the present disclosure comprises the nucleotide sequence of SEQ ID NO:28. In some embodiments, an engineered promoter ablation variant of the present disclosure comprises the nucleotide sequence of SEQ ID NO:29. In some embodiments, an engineered promoter ablation variant of the present disclosure comprises the nucleotide sequence of SEQ ID NO:30. In some embodiments, an engineered promoter ablation variant of the present disclosure comprises the nucleotide sequence of SEQ ID NO:81. In some embodiments, an engineered promoter ablation variant of the present disclosure comprises the nucleotide sequence of SEQ ID NO:82. In some embodiments, an engineered promoter ablation variant of the present disclosure comprises the nucleotide sequence of SEQ ID NO:88. In some embodiments, an engineered promoter ablation variant of the present disclosure comprises the nucleotide sequence of SEQ ID NO:89. In some embodiments, an engineered promoter ablation variant of the present disclosure comprises the nucleotide sequence of SEQ ID NO:90. In some embodiments, an engineered promoter ablation variant of the present disclosure comprises the nucleotide sequence of SEQ ID NO:91. In some embodiments, an engineered promoter ablation variant of the present disclosure comprises the nucleotide sequence of SEQ ID NO:92. In some embodiments, an engineered promoter ablation variant of the present disclosure comprises the nucleotide sequence of SEQ ID NO:96. In some embodiments, an engineered promoter ablation variant of the present disclosure comprises the nucleotide sequence of SEQ ID NO:97. In some embodiments, an engineered promoter ablation variant of the present disclosure comprises the nucleotide sequence of SEQ ID NO:119. In some embodiments, an engineered promoter ablation variant of the present disclosure comprises the nucleotide sequence of SEQ ID NO:120. In some embodiments, an engineered promoter ablation variant of the present disclosure comprises the nucleotide sequence of SEQ ID NO:121. In some embodiments, an engineered promoter ablation variant of the present disclosure comprises the nucleotide sequence of SEQ ID NO:122.

In some embodiments, an engineered macrophage specific promoter or macrophage specific promoter system of the present disclosure comprises the nucleotide sequence of SEQ ID NO:459. In some embodiments, an engineered macrophage specific promoter or macrophage specific promoter system of the present disclosure comprises the nucleotide sequence of SEQ ID NO:460. In some embodiments, an engineered macrophage specific promoter or macrophage specific promoter system of the present disclosure comprises the nucleotide sequence of SEQ ID NO:461. In some embodiments, an engineered macrophage specific promoter or macrophage specific promoter system of the present disclosure comprises the nucleotide sequence of SEQ ID NO:462. In some embodiments, an engineered macrophage specific promoter or macrophage specific promoter system of the present disclosure comprises the nucleotide sequence of SEQ ID NO:463. In some embodiments, an engineered macrophage specific promoter or macrophage specific promoter system of the present disclosure comprises the nucleotide sequence of SEQ ID NO:464. In some embodiments, an engineered macrophage specific promoter or macrophage specific promoter system of the present disclosure comprises the nucleotide sequence of SEQ ID NO:465.

In some embodiments, a promoter ablation variant of the present disclosure does not comprise substitution at the ablation. In some embodiments, a promoter ablation variant of the present disclosure comprises a deletion at the ablation.

In some embodiments, an engineered promoter of the present disclosure comprises a nucleotide sequence selected from the group consisting of SEQ ID NOs: 297-390. In some embodiments, an engineered promoter of the present disclosure comprises the nucleotide sequence of SEQ ID NO: 297. In some embodiments, an engineered promoter of the present disclosure comprises the nucleotide sequence of SEQ ID NO: 298. In some embodiments, an engineered promoter of the present disclosure comprises the nucleotide sequence of SEQ ID NO: 299.

In some embodiments, a engineered promoter of the present disclosure (e.g., a promoter ablation variant or engineered promoter) comprises a nucleotide sequence selected from the group consisting of SEQ ID NOs: 297-390. In some embodiments, an engineered promoter of the present disclosure comprises the nucleotide sequence of SEQ ID NO: 297. In some embodiments, an engineered promoter of the present disclosure comprises the nucleotide sequence of SEQ ID NO: 298. In some embodiments, an engineered promoter of the present disclosure comprises the nucleotide sequence of SEQ ID NO: 299.

In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 297. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 298. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 299. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 300. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 301. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 302. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 303. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 304. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 305. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 306. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 307. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 308. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 309. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 310. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 311. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 312. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 313. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 314. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 315. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 316. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 317. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 318. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 319. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 320. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 321. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 322. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 323. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 324. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 325. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 326. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 327. In some embodiments, an engineered promoter of the present disclosure comprises a nucleotide sequence having at least 80%, at least 85%, at least 90%, at least 95%, at least 96, 97, 98, 99, or 100% sequence identity to SEQ ID NO: 328. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 329. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 330. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 331. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 332. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 333. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 334. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 335. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 336. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 337. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 338. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 339. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 340. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 341. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 342. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 343. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 344. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 345. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 346. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 347. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 348. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 349. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 350. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 351. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 352. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 353. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 354. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 355. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 356. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 357. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 358. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 359. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 360. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 361. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 362. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 363. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 364. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 365. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 366. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 367. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 368. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 369. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 370. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 371. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 372. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 373. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 374. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 375. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 376. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 377. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 378. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 379. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 380. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 381. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 382. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 383. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 384. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 385. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 386. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 387. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 388. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 389. In some embodiments, an engineered promoter of the present disclosure comprises the nucleotide sequence having at least 80%, at least 85%, at least 90%, at least 95%, at least 96%, at least 97%, at least 98%, at least 99%, or 100% sequence identity to SEQ ID NO: 390.

In some embodiments, an engineered promoter of the present disclosure comprises the nucleotide sequence of SEQ ID NO: 300. In some embodiments, an engineered promoter of the present disclosure comprises the nucleotide sequence of SEQ ID NO: 301. In some embodiments, an engineered promoter of the present disclosure comprises the nucleotide sequence of SEQ ID NO: 302. In some embodiments, an engineered promoter of the present disclosure comprises the nucleotide sequence of SEQ ID NO: 303. In some embodiments, an engineered promoter of the present disclosure comprises the nucleotide sequence of SEQ ID NO: 304.

In some embodiments, an engineered promoter of the present disclosure comprises the nucleotide sequence of SEQ ID NO: 305. In some embodiments, an engineered promoter of the present disclosure comprises the nucleotide sequence of SEQ ID NO: 306. In some embodiments, an engineered promoter of the present disclosure comprises the nucleotide sequence of SEQ ID NO: 307. In some embodiments, an engineered promoter of the present disclosure comprises the nucleotide sequence of SEQ ID NO: 308. In some embodiments, an engineered promoter of the present disclosure comprises the nucleotide sequence of SEQ ID NO: 309.

In some embodiments, an engineered promoter of the present disclosure comprises the nucleotide sequence of SEQ ID NO: 310. In some embodiments, an engineered promoter of the present disclosure comprises the nucleotide sequence of SEQ ID NO: 311. In some embodiments, an engineered promoter of the present disclosure comprises the nucleotide sequence of SEQ ID NO: 312. In some embodiments, an engineered promoter of the present disclosure comprises the nucleotide sequence of SEQ ID NO: 313. In some embodiments, an engineered promoter of the present disclosure comprises the nucleotide sequence of SEQ ID NO: 314. In some embodiments, an engineered promoter of the present disclosure comprises the nucleotide sequence of SEQ ID NO: 315. In some embodiments, an engineered promoter of the present disclosure comprises the nucleotide sequence of SEQ ID NO: 316. In some embodiments, an engineered promoter of the present disclosure comprises the nucleotide sequence of SEQ ID NO: 317. In some embodiments, an engineered promoter of the present disclosure comprises the nucleotide sequence of SEQ ID NO: 318. In some embodiments, an engineered promoter of the present disclosure comprises the nucleotide sequence of SEQ ID NO: 319. In some embodiments, an engineered promoter of the present disclosure comprises the nucleotide sequence of SEQ ID NO: 320. In some embodiments, an engineered promoter of the present disclosure comprises the nucleotide sequence of SEQ ID NO: 321. In some embodiments, an engineered promoter of the present disclosure comprises the nucleotide sequence of SEQ ID NO: 322.

In some embodiments, an engineered promoter of the present disclosure comprises the nucleotide sequence of SEQ ID NO: 323. In some embodiments, an engineered promoter of the present disclosure comprises the nucleotide sequence of SEQ ID NO: 324. In some embodiments, an engineered promoter of the present disclosure comprises the nucleotide sequence of SEQ ID NO: 325. In some embodiments, an engineered promoter of the present disclosure comprises the nucleotide sequence of SEQ ID NO: 326. In some embodiments, an engineered promoter of the present disclosure comprises the nucleotide sequence of SEQ ID NO: 327.

In some embodiments, an engineered promoter of the present disclosure comprises the nucleotide sequence of SEQ ID NO: 328. In some embodiments, an engineered promoter of the present disclosure comprises the nucleotide sequence of SEQ ID NO: 329. In some embodiments, an engineered promoter of the present disclosure comprises the nucleotide sequence of SEQ ID NO: 330. In some embodiments, an engineered promoter of the present disclosure comprises the nucleotide sequence of SEQ ID NO: 331. In some embodiments, an engineered promoter of the present disclosure comprises the nucleotide sequence of SEQ ID NO: 332. In some embodiments, an engineered promoter of the present disclosure comprises the nucleotide sequence of SEQ ID NO: 333. In some embodiments, an engineered promoter of the present disclosure comprises the nucleotide sequence of SEQ ID NO: 334. In some embodiments, an engineered promoter of the present disclosure comprises the nucleotide sequence of SEQ ID NO: 335. In some embodiments, an engineered promoter of the present disclosure comprises the nucleotide sequence of SEQ ID NO: 336. In some embodiments, an engineered promoter of the present disclosure comprises the nucleotide sequence of SEQ ID NO: 337. In some embodiments, an engineered promoter of the present disclosure comprises the nucleotide sequence of SEQ ID NO: 338. In some embodiments, an engineered promoter of the present disclosure comprises the nucleotide sequence of SEQ ID NO: 339. In some embodiments, an engineered promoter of the present disclosure comprises the nucleotide sequence of SEQ ID NO: 340. In some embodiments, an engineered promoter of the present disclosure comprises the nucleotide sequence of SEQ ID NO: 341. In some embodiments, an engineered promoter of the present disclosure comprises the nucleotide sequence of SEQ ID NO: 342. In some embodiments, an engineered promoter of the present disclosure comprises the nucleotide sequence of SEQ ID NO: 343. In some embodiments, an engineered promoter of the present disclosure comprises the nucleotide sequence of SEQ ID NO: 344. In some embodiments, an engineered promoter of the present disclosure comprises the nucleotide sequence of SEQ ID NO: 345. In some embodiments, an engineered promoter of the present disclosure comprises the nucleotide sequence of SEQ ID NO: 346. In some embodiments, an engineered promoter of the present disclosure comprises the nucleotide sequence of SEQ ID NO: 347. In some embodiments, an engineered promoter of the present disclosure comprises the nucleotide sequence of SEQ ID NO: 348. In some embodiments, an engineered promoter of the present disclosure comprises the nucleotide sequence of SEQ ID NO: 349. In some embodiments, an engineered promoter of the present disclosure comprises the nucleotide sequence of SEQ ID NO: 350. In some embodiments, an engineered promoter of the present disclosure comprises the nucleotide sequence of SEQ ID NO: 351. In some embodiments, an engineered promoter of the present disclosure comprises the nucleotide sequence of SEQ ID NO: 352. In some embodiments, an engineered promoter of the present disclosure comprises the nucleotide sequence of SEQ ID NO: 353. In some embodiments, an engineered promoter of the present disclosure comprises the nucleotide sequence of SEQ ID NO: 354. In some embodiments, an engineered promoter of the present disclosure comprises the nucleotide sequence of SEQ ID NO: 355. In some embodiments, an engineered promoter of the present disclosure comprises the nucleotide sequence of SEQ ID NO: 356. In some embodiments, an engineered promoter of the present disclosure comprises the nucleotide sequence of SEQ ID NO: 357. In some embodiments, an engineered promoter of the present disclosure comprises the nucleotide sequence of SEQ ID NO: 358. In some embodiments, an engineered promoter of the present disclosure comprises the nucleotide sequence of SEQ ID NO: 359. In some embodiments, an engineered promoter of the present disclosure comprises the nucleotide sequence of SEQ ID NO: 360. In some embodiments, an engineered promoter of the present disclosure comprises the nucleotide sequence of SEQ ID NO: 361. In some embodiments, an engineered promoter of the present disclosure comprises the nucleotide sequence of SEQ ID NO: 362. In some embodiments, an engineered promoter of the present disclosure comprises the nucleotide sequence of SEQ ID NO: 363. In some embodiments, an engineered promoter of the present disclosure comprises the nucleotide sequence of SEQ ID NO: 364. In some embodiments, an engineered promoter of the present disclosure comprises the nucleotide sequence of SEQ ID NO: 365. In some embodiments, an engineered promoter of the present disclosure comprises the nucleotide sequence of SEQ ID NO: 366. In some embodiments, an engineered promoter of the present disclosure comprises the nucleotide sequence of SEQ ID NO: 367. In some embodiments, an engineered promoter of the present disclosure comprises the nucleotide sequence of SEQ ID NO: 368. In some embodiments, an engineered promoter of the present disclosure comprises the nucleotide sequence of SEQ ID NO: 369. In some embodiments, an engineered promoter of the present disclosure comprises the nucleotide sequence of SEQ ID NO: 370. In some embodiments, an engineered promoter of the present disclosure comprises the nucleotide sequence of SEQ ID NO: 371. In some embodiments, an engineered promoter of the present disclosure comprises the nucleotide sequence of SEQ ID NO: 372. In some embodiments, an engineered promoter of the present disclosure comprises the nucleotide sequence of SEQ ID NO: 373. In some embodiments, an engineered promoter of the present disclosure comprises the nucleotide sequence of SEQ ID NO: 374. In some embodiments, an engineered promoter of the present disclosure comprises the nucleotide sequence of SEQ ID NO: 375. In some embodiments, an engineered promoter of the present disclosure comprises the nucleotide sequence of SEQ ID NO: 376. In some embodiments, an engineered promoter of the present disclosure comprises the nucleotide sequence of SEQ ID NO: 377. In some embodiments, an engineered promoter of the present disclosure comprises the nucleotide sequence of SEQ ID NO: 378. In some embodiments, an engineered promoter of the present disclosure comprises the nucleotide sequence of SEQ ID NO: 379. In some embodiments, an engineered promoter of the present disclosure comprises the nucleotide sequence of SEQ ID NO: 380. In some embodiments, an engineered promoter of the present disclosure comprises the nucleotide sequence of SEQ ID NO: 381. In some embodiments, an engineered promoter of the present disclosure comprises the nucleotide sequence of SEQ ID NO: 382. In some embodiments, an engineered promoter of the present disclosure comprises the nucleotide sequence of SEQ ID NO: 383. In some embodiments, an engineered promoter of the present disclosure comprises the nucleotide sequence of SEQ ID NO: 384. In some embodiments, an engineered promoter of the present disclosure comprises the nucleotide sequence of SEQ ID NO: 385. In some embodiments, an engineered promoter of the present disclosure comprises the nucleotide sequence of SEQ ID NO: 386. In some embodiments, an engineered promoter of the present disclosure comprises the nucleotide sequence of SEQ ID NO: 387. In some embodiments, an engineered promoter of the present disclosure comprises the nucleotide sequence of SEQ ID NO: 388. In some embodiments, an engineered promoter of the present disclosure comprises the nucleotide sequence of SEQ ID NO: 389. In some embodiments, an engineered promoter of the present disclosure comprises the nucleotide sequence of SEQ ID NO: 390.

In some embodiments, an engineered promoter of the present disclosure comprises a nucleotide sequence as set forth in GTTAAGTGGCTAGGGATAACATTGAGGCACTAAAGCATTATTGGTTCTGCAGTCA AGGGTAGGATAGATTGTTTTTTTTTTTTT (SEQ ID NO: 482), and a nucleotide sequence as set forth in

(SEQ ID NO: 483)
TTTGTGGTTTTATTGGTTTTCATATTACAAACAAAGAAACTAGAAAATGA
AACCATTCCAAAAGTGGAAGTAATTTCTCA.

In some embodiments, an engineered promoter of the present disclosure comprises a nucleotide sequence as set forth in GCTCTTCTAAAAATATGCGAAATGAGGTTTTTAGGGAGGTGTAGGTATGGCTGAA GAAAATCAAGGTGAATGAAGACAAGATCAATTGAGAATGTAGTTTCAGAAATAG CAAAGAAGCCAAAGTTTGAGGAAGTTAAGTGGCTAGGGATAACATTGAGGCACT AAAGCATTATTGGTTCTGCAGTCAAGGGTAGGATAGATTGTTTTTTTTTTTTTTGA GACGGAGTCTCACTCTGCTGCCCAGGC (SEQ ID NO: 484), a nucleotide sequence as set forth in ATTTTGGTTTCAGTTTTCCTTAC (SEQ ID NO: 240), and a nucleotide sequence as set forth in

(SEQ ID NO: 483)
TTTGTGGTTTTATTGGTTTTCATATTACAAACAAAGAAACTAGAAAATGA
AACCATTCCAAAAGTGGAAGTAATTTCTCA.

In some embodiments, an engineered promoter of the present disclosure comprises a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256. In some embodiments, an engineered promoter of the present disclosure comprises a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256, and does not comprise at least one repressive element selected from: SEQ ID NO: 226, SEQ ID NO: 234, SEQ ID NO: 236, SEQ ID NO: 238, SEQ ID NO: 246, and SEQ ID NO: 252. In some embodiments, an engineered promoter of the present disclosure comprises a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256, and does not comprise repressive elements as set forth in SEQ ID NO: 226, SEQ ID NO: 234, SEQ ID NO: 236, SEQ ID NO: 238, SEQ ID NO: 246, and SEQ ID NO: 252. In some embodiments, an engineered promoter further comprising a sixth transcriptional activating element as set forth in SEQ ID NO: 224. In some embodiments, an engineered promoter further comprises a seventh transcriptional activating element as set forth in SEQ ID NO: 258.

In some embodiments, an engineered promoter of the present disclosure comprises, from 5′ to 3′, a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256. In some embodiments, an engineered promoter of the present disclosure comprises, from 5′ to 3′, a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256, and does not comprise at least one repressive element selected from: SEQ ID NO: 226, SEQ ID NO: 234, SEQ ID NO: 236, SEQ ID NO: 238, SEQ ID NO: 246, and SEQ ID NO: 252. In some embodiments, an engineered promoter of the present disclosure comprises, from 5′ to 3′, a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256, and does not comprise repressive elements as set forth in SEQ ID NO: 226, SEQ ID NO: 234, SEQ ID NO: 236, SEQ ID NO: 238, SEQ ID NO: 246, and SEQ ID NO: 252. In some embodiments, an engineered promoter further comprising a sixth transcriptional activating element as set forth in SEQ ID NO: 224. In some embodiments, an engineered promoter further comprises a seventh transcriptional activating element as set forth in SEQ ID NO: 258.

In some embodiments, an engineered promoter of the present disclosure does not comprise SEQ ID NO: 228, SEQ ID NO: 230, SEQ ID NO: 232, SEQ ID NO: 242, SEQ ID NO: 244, SEQ ID NO: 248, and SEQ ID NO: 250.

In some embodiments, an engineered promoter of the present disclosure comprises a first transcriptional activating element as set forth in SEQ ID NO: 268 and a second transcriptional activating element as set forth in SEQ ID NO: 270. In some embodiments, an engineered promoter of the present disclosure comprises a first transcriptional activating element as set forth in SEQ ID NO: 268 and a second transcriptional activating element as set forth in SEQ ID NO: 270, and does not comprise at least one repressive element selected from: SEQ ID NO: 260, SEQ ID NO: 262, SEQ ID NO: 264, SEQ ID NO: 266, SEQ ID NO: 272, and SEQ ID NO: 391. In some embodiments, an engineered promoter of the present disclosure comprises a first transcriptional activating element as set forth in SEQ ID NO: 268 and a second transcriptional activating element as set forth in SEQ ID NO: 270, and does not comprise repressive elements as set forth in SEQ ID NO: 260, SEQ ID NO: 262, SEQ ID NO: 264, SEQ ID NO: 266, SEQ ID NO: 272, and SEQ ID NO: 391. In some embodiments, an engineered promoter further comprises a third transcriptional activating element as set forth in SEQ ID NO: 291. In some embodiments, an engineered promoter further comprises a fourth activating element as set forth in SEQ ID NO: 295.

In some embodiments, an engineered promoter of the present disclosure comprises, from 5′ to 3′, a first transcriptional activating element as set forth in SEQ ID NO: 268 and a second transcriptional activating element as set forth in SEQ ID NO: 270. In some embodiments, an engineered promoter of the present disclosure comprises, from 5′ to 3′, a first transcriptional activating element as set forth in SEQ ID NO: 268 and a second transcriptional activating element as set forth in SEQ ID NO: 270, and does not comprise at least one repressive element selected from: SEQ ID NO: 260, SEQ ID NO: 262, SEQ ID NO: 264, SEQ ID NO: 266, SEQ ID NO: 272, and SEQ ID NO: 391. In some embodiments, an engineered promoter of the present disclosure comprises, from 5′ to 3′, a first transcriptional activating element as set forth in SEQ ID NO: 268 and a second transcriptional activating element as set forth in SEQ ID NO: 270, and does not comprise repressive elements as set forth in SEQ ID NO: 260, SEQ ID NO: 262, SEQ ID NO: 264, SEQ ID NO: 266, SEQ ID NO: 272, and SEQ ID NO: 391. In some embodiments, an engineered promoter further comprises a third transcriptional activating element as set forth in SEQ ID NO: 291. In some embodiments, an engineered promoter further comprises a fourth activating element as set forth in SEQ ID NO: 295.

In some embodiments, an engineered promoter of the present disclosure does not comprise SEQ ID NO: 260. In some embodiments, an engineered promoter does not comprise SEQ ID NO: 266. In some embodiments, an engineered promoter of the present disclosure does not comprise SEQ ID NO: 260 or SEQ ID NO: 266.

In some embodiments, an engineered promoter of the present disclosure comprises at least one, at least two, at least three, at least four, at least five, at least six, at least seven, at least eight, at least nine, at least ten tandem repeats of one or more activating elements. In some embodiments, an engineered promoter comprises at least one tandem repeat of one or more activating elements. In some embodiments, an engineered promoter comprises at least two tandem repeats of one or more activating elements. In some embodiments, an engineered promoter comprises at least three tandem repeats of one or more activating elements. In some embodiments, an engineered promoter comprises at least four tandem repeats of one or more activating elements. In some embodiments, an engineered promoter comprises at least five tandem repeats of one or more activating elements. In some embodiments, two or more tandem repeats are contiguous. In some embodiments, two or more tandem repeats are non-contiguous.

In some embodiments, an engineered promoter of the present disclosure comprises at least one, at least two, at least three, at least four, at least five, at least six, at least seven, at least eight, at least nine, at least ten tandem repeats of SEQ ID NO: 268 and SEQ ID NO: 270. In some embodiments, an engineered promoter of the present disclosure comprises at least one tandem repeat of SEQ ID NO: 268 and SEQ ID NO: 270. In some embodiments, an engineered promoter of the present disclosure comprises at least two tandem repeats of SEQ ID NO: 268 and SEQ ID NO: 270. In some embodiments, an engineered promoter of the present disclosure comprises at least three tandem repeats of SEQ ID NO: 268 and SEQ ID NO: 270. In some embodiments, an engineered promoter of the present disclosure comprises at least four tandem repeats of SEQ ID NO: 268 and SEQ ID NO: 270. In some embodiments, an engineered promoter of the present disclosure comprises at least five tandem repeats of SEQ ID NO: 268 and SEQ ID NO: 270.

In some embodiments, an engineered promoter of the present disclosure comprises at least one regulatory element, such as an enhancer sequence. Exemplary polarization-specific enhancers are disclosed herein. In some embodiments, the engineered macrophage-specific promoter comprises at least 2, at least 3, at least 4, or at least 5 regulatory elements. In some embodiments, each of the at least 5 regulatory elements are different. In some embodiments, each of the at least 5 regulatory elements are the same.

In some embodiments, an engineered promoter of the present disclosure further comprises a minimal promoter sequence. The minimal promoter may be operably linked to the at least one regulatory element. In some embodiments, the minimal promoter is derived from a promoter selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, inducer molecule responsive promoters, CMV, EFS, SCP3, YB-SCP3, SFFV, SV40, MND, PGK, UbC, hEF1aV1, hCAGG, hEF1aV2, hACTb, heIF4A1, hGAPDH, hGRP78, hGRP94, hHSP70, hKINb, hUBIb, and tandem repeats thereof. In some embodiments, a minimal promoter is or comprises a YBTATA promoter. In some embodiments, a minimal promoter is or comprises a minCMV promoter. In some embodiments, a minimal promoter is or comprises a minTK promoter. In some embodiments, a minimal promoter is or comprises a SCP3 promoter. In some embodiments, a minimal promoter is or comprises a YB-SCP3 promoter. In some embodiments, a minimal promoter comprises a flanking spacer sequence at the N- and/or C-terminus of the minimal promoter. Exemplary minimal promoter sequences are shown in Table 10. In some embodiments, an engineered promoter of the present disclosure comprises a minimal promoter (e.g., any minimal promoter described herein, e.g., those provided in Table 10) and an engineered polarization specific enhancer (e.g., any engineered polarization specific enhancer described herein, e.g., those provided in Table 3 or Table 8).

TABLE 3
Additional Engineered Polarization-Specific Enhancers.

| SEQ | | Polarization |
|---|---|---|
| ID | | State |
| NO | Enhancer DNA Sequence | Specificity |
| 297 | GATGAGTCATCACGTCCGAACACTTCCGCCTTACAGCTGG | M1 |
| | GAGCAGGTGCAGTCGCGTAATCACGTCACCATTATCCAAG | |
| | GGGATTCCCCT | |
| 298 | TCACTTTCACTTTCTTATAACTGCACCTGCTCCCTACGAC | M1 |
| | TCCGGGGAATTCAGTCCACAAGATGAGTCATCAGCTGGCT | |
| | AGTCACGTGAC | |
| 299 | GGGAAACGGAAACCGAAACTGTATAGGGGAATCCCCTACG | M1 |
| | TTCATTGCGTGGGCGTTGGAGTTGTATCACGTCACCTTGC | |
| | GTTCTCACTTCCTGCTC | |
| 300 | TCAGTTTCATTTTCCAACTAAGATTGTGCAATATTCCACT | M1 |
| | TAGTCACGTGACTTGGTCCCACACTTCCGCCTAAGAGGGA | |
| | AACGGAAACCGAAACTG | |
| 301 | GGGAAACGGAAACCGAAACTGAAGTTGCGTGGGCGTAGGT | M1 |
| | AGTCTGCACCTGCTCCCTTAGAAGAGCAGGAAGTGAGAGT | |
| | CAGAGGTCACTGCAGGTCA | |
| 302 | GGGAGCAGGTGCAGATGAAGAACGCCCACGCATTGATGAC | M1 |
| | TTTTCCAGGAAAATCACAGTTTCGGTTTCCGTTTCCCTAG | |
| | GAAAATGAAACTGA | |
| 303 | GGAAAATGAAACTGAATCCTCTCACTTCCTGCTCTAGGCA | M1 |
| | GGGAGCAGGTGCAGTGATGCTGATGACTCATCATCGAGTA | |
| | ACGAAACCGAAACT | |
| 304 | AACAGCTGTTTCTCAGGGAAACGGAAACCGAAACTGTATT | M1 |
| | AGGCGGAAGTGTTGCTTATCACTTTCACTTTCAACGCTAA | |
| | TCACGTCACC | |
| 305 | CACTTCCGCCTTAGAGGAAACGAAACCGAAACTATTCTAT | M1 |
| | ATCACGTCACCTATCTACAAGGGGATTCCCCTAAGAGATC | |
| | AGTTTCATTTTCC | |
| 306 | AGGGGAATCCCCTTGTCGAATGATGAGTCATCTACGTCTA | M1 |
| | ACGAAACCGAAACTATACTCAGGTGACGTGATTGCTCAAG | |
| | GAAAATGAAACTGA | |
| 307 | GATGAGTCATCTGGTCAGAGGTCACTGCAGGTCATTACTG | M1 |
| | TCACGTGACACCAGTGATACGCCCACGCAATACAACAGTT | |
| | TCATTATGACTC | |
| 308 | GTCACGTGACACATCTAGGAAAATGAAACTGAATGGGAAA | M1 |
| | CGGAAACCGAAACTGTTAGTTTCGGTTTCGTTAGGTTGTG | |
| | AATTCCCCGG | |
| 309 | TGACCTGCAGTGACCTCTGTCAAGGGGATTCCCCTAATCA | M1 |
| | GTTTCGGTTTCCGTTTCCCTCAGAGTCACGTGACTCGTCG | |
| | AGGTAACAGCTGTT | |
| 310 | GGGAAACGGAAACCGAAACTGATGAAGGCGGAAGTGTGTT | M1 |
| | CGACACCGGGGAATTCATATCAAATATTGCACAATCTAGT | |
| | CTGGAAAATGAAACTGA | |
| 311 | GAAAGTGAAAGTGAATTTCGTCCGGGGAATTCTCAGACTT | M1 |
| | AGATGAGTCATCTTCCATAGTACGCCCACGCATTAGTATT | |
| | AAACAGCTGTT | |
| 312 | GAATTCCCCGGACAAGCCTTGATGACTCATCTAGTCAGTT | M1 |
| | TCGGTTTCCGTTTCCCAATTGACCTGCAGTGACCTCTGAA | |
| | AGAAAGTGAAAGTGA | |
| 313 | GACACCTGTTTGCGGCTATAAACAGCTGTTAGTCGGGTTT | M1 |
| | ACCAGCTGCTTCTCGTCTGAAAGTGAAAGGGATAACATGG | |
| | AAGAGAAAGTGA | |
| 314 | CACTTCCGCCTTAGAGGGAAACGGAAACCGAAACTGACAA | M2c |
| | GGGGAATCCCCTTCATGTCTGCACCTGCTCCCAAGGCGAG | |
| | GTGACGTGAT | |
| 315 | AGGGGATTCCCCTTGGCGGCTTTTCCTGGAAATACGTTAG | M2c |
| | GGAGCAGGTGCAGAGCGAAGTTTCGGTTTCGTTTCGTTAT | |
| | AGGCGGAAGTG | |
| 316 | ATCACGTCACCTATCACTCTCACTTCCTGCTCAAGGCTAG | M2c |
| | ATGACTCATCATTTCAGAGGTCACTGCAGGTCATAACAGT | |
| | TTCGGTTTCCGTTTCCC | |
| 317 | TTTCCAGGAAAATTCTTAAAGATGACTCATCTAGCGATTC | M2c |
| | AGTTTCATTTTCCTGCGCTAGGTGACGTGATTTAAAGCTA | |
| | GGGGAATCCCCT | |
| 318 | AACAGCTGTTATAGCGCAATGCGTGGGCGTATTAGGGAAA | M2c |
| | CGGAAACCGAAACTGATCTCCGGGGAATTCAAGTGCAAGA | |
| | TTGTGCAATA | |
| 319 | GGAAAATGAAACTGATGAATTTAACAGCTGTTTGTACATT | M2c |
| | GAAAGTGAAAGTGAAAAACTAGAATTCCCCGGATTAGGGA | |
| | AACGGAAACCGAAACTG | |
| 320 | CTGCACCTGCTCCCACCTATCTCCACCTGCCGCCTCCGTT | M2c |
| | CTGCACCTGCCCTCAGCATAACGAAACCGAAACTTTCCAA | |
| | TCGAAAGCGAAACA | |
| 321 | CTGCACCTGCCCTCATGTCACCGCACCTGCCGCCATCCCA | M2c |
| | CTCCACCTGCCGCCACGGCATTTTCTAGGAAGAGTCCTCG | |
| | ACTTCCGGGAAG | |
| 322 | GGAAAATGAAACTGAATTTCAGTTTCATTATGACTCACAG | M2c |
| | CTGTCACGTGACAGAGAGTTTCACTTTCACTTTCATTGCT | |
| | CTGCACCTGCTCCC | |
| 323 | TGCGTGGGCGTTATCGGAGGGAGCAGGTGCAGTTGTGAAA | M2c |
| | AGATCAAAGGAATAAACGTGATGACTCATCATATTGACCT | |
| | GCAGTGACCTCTG | |
| 324 | GGGAGCAGGTGCAGTGTCCCTCACTTCCGCCTTCCGATAT | M2c |
| | CAGTTTCATTTTCCAGTTCTAGGGGAATCCCCTAGAGGGA | |
| | AACGGAAACCGAAACTG | |
| 325 | AGTTTCGGTTTCGTTTTCAGTTTCGGTTTCCGTTTCCCAA | M2c |
| | GTTGTCACGTGACAACGCTACTAGGCGGAAGTGTATCTTG | |
| | AGTCATAATGAAACTG | |
| 326 | AGGCGGAAGTGTTGTATCAGTTTCATTATGACTCACCGTT | M2c |
| | ATTGCACAATCACGACATCTCACTTCCTGCTCAGTTGACC | |
| | TGCAGTGACCTCTG | |
| 327 | ACGCCCACGCAAGTCTCATCACTTTCACTTTCTACCCAGA | M2c |
| | GCAGGAAGTGAGTACGTATCCGGGGAATTCAGTAAACGAG | |
| | TCACGTGAC | |
| 328 | GTCACGTGACATAGTACTAGGGGAATCCCCTAAAGTCTAT | M2c |
| | GCGTGGGCGTAGTCGATATTTTCCTGGAAATCGTCAGAGG | |
| | TCACTGCAGGTCA | |
| 329 | GTCACGTGACACCAACAGTTTCGGTTTCCGTTTCCCAATA | M2c |
| | GGGGAATCCCCTAGAACGCTGATGAGTCATCAAAATCTCT | |
| | CACTTCCTGCTC | |
| 330 | CCGCACCTGCCGCCTTGCCACTCCACCTGCCGCCAAACAA | M2c |
| | AGGTCACCTCAGGCCAACTCTGAGGGCACTAAAGGTCAAT | |
| | ACAGAGGTCACTGCAGGTCA | |
| 331 | GTCACGTGCCTCCAGCAGTCTGAGTCAGCATGATTAGCAA | M2c |
| | AGTAAACAAATATGTAGAGTCATAATGAAACTGAATCTAA | |
| | AAGATAAGAA | |
| 332 | CAGTTTCATTATGACTCAGAAACAATGTGCAGTGTTTGCA | M2c |
| | GATGTCACGTGCCTCCTCGTTAGTTTCGGTTTCGGTCGCT | |
| | TGTAAATGACT | |
| 333 | TAGTTTCGGTTTCGGTACAGAGTCATAATGAAACTGAGAC | M2c |
| | AAAAGGTGTGAAAACCATTCGCATAGTCATTTACCCTAAG | |
| | CAGGCACGTGAC | |
| 334 | AAGTAAACAAATTGAAAGTCCACTTCCGGTCTAATACCTT | M2c |
| | GTCACGTGCCTCGTCAATAAAATAAACAATTTACCTACAT | |
| | ATCAGGTTAC | |
| 335 | GGCACGTGACATAGCGACTCAAGTCATTTCGTCTGTGAAT | M2c |
| | GCACCTGTTATACTACTCCACTTCCGGTCTTATGTAAATT | |
| | CTTATCTTT | |
| 336 | CCACTTCCGGTCTTGTCCGTAAACAGGTGCAAGTAACTAA | M2c |
| | GTAACCTGATAATACGCGTACTAATGACTTATGTCCCTGT | |
| | GGCACGTGAC | |
| 337 | GTAACCTGATATATCCATTATGCTGACTCAGATCTCAAAC | M2c |
| | AATGTGCAGTGTTTATCTCCACTTCCGGTCTACTGACAAG | |
| | TCACGTGCC | |
| 338 | CCGAAACCGAAACTAATAGCTTATATTTACATAATCGATG | M2c |
| | ACAAAATGACTTCCTCGAATTAAATAAACAATTTAGCATC | |
| | CTGTCACGTGCC | |
| 339 | GTAACCTGATATCCCCGAATGGCACGTGACTTCACTACCG | M2c |
| | AAACCGAAACTAAACGTCTTTCTTATCTTTACTTTATCAT | |
| | TTGTTTACTT | |
| 340 | CAAAGGTCAGACTCGATCAAAATAAACAATTTAAGTTATA | M2c |
| | GTCACGTGCCTACTTTATAGTTTCGGTTTCGGATGTGACA | |
| | GCTAATGGA | |
| 341 | TTTTCCATCAGTGCGTAGCATGACTCAGCATTCGTAGACA | M2c |
| | AGTCATTTATACTAATAAAATAAACAATTAGACTTGTTGT | |
| | CACGTGCC | |
| 342 | TGCACCTGTTTCATTCCATGCTGAGTCATGCAGGACCCTG | M2c |
| | GCACGTGACATTTTATAACACTGCACATTGTTAGTGGCTG | |
| | CTAATGGA | |
| 343 | ATATTTACATAATACTAGCGCTTGGAAAAACACGTTCATA | M2c |
| | GACCGGAAGTGGTCTTACAGTTTCATTATGACTCTTGGCA | |
| | CAAAGGTCAG | |
| 344 | TTTGTTTACTTTTTAAACATTGCTGACTCAGACAGTGACA | M2c |
| | GGAAGCAGATGTGAGTCGTAGACCGGAAGTGGTAACTGAG | |
| | TCATAATGAAACTG | |
| 345 | CACATCTGCTTCCTGTAGAGTCCACTTCCGGTCTAGTACG | M2c |
| | AACAAAGGTCAGTGTAGTTTAGTTTCGGTTTCGGACTTCA | |
| | GTTTCATTATGACTC | |
| 346 | GAGTCATAATGAAACTGAAGTCCGAAACCGAAACTAACAT | M2c |
| | AAGCATGACTCAGCATCGGGGTATATCAGGTTACTCTTCG | |
| | CGTATATTTACATA | |
| 347 | CAGTTTCATTATGACTCTCGCTAAAGGTGTGAAATCCCGA | M2c |
| | TTTTATGTAAATATTAATTCTCTGTAGTCATTATAGCAAT | |
| | AAGTTCCATTAGC | |
| 348 | TTTTCCAAAGCCGATCAGTTTCATTATGACTCAACACAGG | M2c |
| | AAGCAGATGTGTCATAGAAAAGATAAGAAACAACATTTAA | |
| | CAGGTGCA | |
| 349 | AACACTGCACATTGTTTAAAGAAAAGATAAGAAACCGAGT | M2c |
| | CAGGAAGCAGATGTGACTAATAGTAACCTGATATTGAAAC | |
| | AGTTTCATTATGACTC | |
| 350 | TTTCACACCTTTTGGGTTAACTGAGTCAGCATTACGACTC | M2c |
| | AAAATGACTTCACCAGATTGCATGACTCAGCAAATGTGAG | |
| | TCATAATGAAACTG | |
| 351 | AACACTGCACATTGTTTCTCAGTTTCATTATGACTCTGGT | M2c |
| | GTGTAACCTGATATGGTGTTCACATCTGCTTCCTGAATCA | |
| | TATAAATGACT | |
| 352 | CCACTTCCGGTCTTTTGACAAATTGTTTATTTTTTTAGAG | M2c |
| | ATGCACCTGTTACACTAGAAGTAACCTGATAAAGAACATA | |
| | CAAAGGTCAG | |
| 353 | TCCATTAGCAGGCCTAATAATTGTTTATTTATAGATCTAC | M2c |
| | TGACCTTTGAATAGCGCCTAAATGACTTCGTTGCGATCCA | |
| | CTTCCGGTCT | |
| 354 | AGACCGGAAGTGGACGTAACACATCTGCTTCCTGTAGTTA | M2c |
| | AAATAAACAATTAAAGTCGAGTAACCTGATATCGTGTAAC | |
| | AATGTGCAGTGTT | |
| 355 | AGACCGGAAGTGGAGCCGTCTTGCACCTGTTATCCGTGTA | M2c |
| | TATCAGGTTACTTCAGTGAACTTTTTCCATAACGTTAAAC | |
| | AATGTGCAGTGTT | |
| 356 | GCATGACTCAGCATACTGTTTTATCAGGTTACAAGCCGAA | M2c |
| | TGTAATGACTACATTTAATAAGACCGGAAGTGGAGCTTAC | |
| | TTTTCACACCTT | |
| 357 | AATGACTACACAACTACGAGCTAATGGAAGCAACCCGTTG | M2c |
| | CTGACTCAGTAGTACAAAAAGTAAACAAAAGTCGCCTTTA | |
| | TGTAAATAT | |
| 358 | TTTGTTTACTTTTCCTATTAATTGTTTATTTAAGTAGCAT | M2c |
| | AACAGGTGCATGCCCTACCGAAACCGAAACTATGCATTTA | |
| | AAGATAAGAA | |
| 359 | AAAGATAAGAAACCTCTTAAAAGTAAACAAAAAGTATAGT | M2c |
| | TTTCACACCTTTATATTCTAAATAAACAATTATGGTACAA | |
| | TCCATTAGC | |
| 360 | CCGAAACCGAAACTATGCTCTAAAGGTGTGAAAATCGACG | M2c |
| | ATGCTGAGTCATGCACTATATTAAGTAAACAAAACTTAAT | |
| | CAGGAAGCAGATGTG | |
| 361 | TTTGTTTACTTACCAAATCAGTAACCTGATATACGACATA | M2c |
| | CAAAGGTCAGTGTCACTGCGTTTTTCCAACCGTAAGTTAC | |
| | TGAGTCAGCA | |
| 362 | AGTCATTTAGGTCCTTCGATTGGAAAAATCACAAGGGTTG | M2c |
| | CTGACTCAGTATAGCGAAAATAAACAATTAATTCACAAAA | |
| | GATAAGAA | |
| 363 | CTGAGTCAGCAATCTGTGAAATTGTTTATTTTCGTACACT | M2c |
| | TTTTTCCAATCTTAGTTCCGAAACCGAAACTAAAGCACAG | |
| | GAAGCAGATGTG | |
| 364 | AACAATGTGCAGTGTTTTGGTTGCTGAGTCATGCACGCAT | M2c |
| | TAGTTTCGGTTTCGGTGAGACTAACAGGTGCATCACGAAA | |
| | TAATTGTTTATTT | |
| 365 | CTGAGTCAGCAACTATGGTGAATTTTCCAAAGTACATCAC | M2c |
| | TGCTAATGGAAACCTGTATTCTGACCTTTGATAGTGCATA | |
| | AATAAACAATT | |
| 366 | TATCAGGTTACAAGACTCTAAAGGTGTGAAAACCCAAGTT | M2c |
| | GCTGAGTCATGCTGTCGTAAAATAAACAATTTCTGTAGAT | |
| | CTGACCTTTG | |
| 367 | AACACTGCACATTGTTTATCCTAAAGATAAGAAAGCGGTC | M2c |
| | ACAATTTTCCATAAACATAGCGTGCTAATGGAATAGTCTA | |
| | TGCATGACTCAGCA | |
| 368 | TGGAAAAACGAGTGCTATGCTGAGTCATGCTCTAAATAAA | M2c |
| | CAGGTGCATAGGAGTCCGAAACCGAAACTAAACGTCCGTA | |
| | GTCATT | |
| 369 | AACAATGTGCAGTGTTTCGAGGTCTGACCTTTGTCACAAT | M2c |
| | GACTAGTCATTATCCGTCTACCGAAACCGAAACTAACGAA | |
| | ATATCCATTAGC | |
| 370 | CTAGTTTGGCAACAGGCATCTTCCACCCACCTGGGAGGCA | M2c |
| | GGGTTCAACACTCTGCCTCTGACCTTTTTTCCTTCCTCTG | |
| | CCACCTGCTTAGGCAGCCAG | |
| 371 | AGATGCACTTCGCTTTTGTTATTTTTTTAGTAGAGAGGGG | M2c |
| | GTTTCACTGGTTTAGCCAGAACAGTCTCGATCTCCTGACC | |
| | TCGTGATCCGCCCACCTCGG | |
| 372 | GAGTCATAATGAAACTGAACTCTCTGACCTTTGTCTCTCA | M1 |
| | CACATCTGCTTCCTGAAGAGCTGGCACGTGACTTTATCTA | |
| | ATTCTTATCTTT | |
| 373 | TATCAGGTTACATGGAGTGTGTCACGTGCCACGAAGTTAA | M1 |
| | AAGATAAGAAAGCTTTTTATATGGAAAATCTCGACTAAAG | |
| | ACCGGAAGTGG | |
| 374 | TATGTAAATATTAAAGGTATTATCAGGTTACTAGTATGTA | M1 |
| | AATAAACAATTTTCTCCGTAGGCACGTGACTCGCGACAAT | |
| | TCTTATCTTT | |
| 375 | TAGTTTCGGTTTCGGTATCGCATATGTAAATATATCCATG | M1 |
| | TATGCACCTGTTTGCATTGCTAGTCACGTGCCTATAACAT | |
| | GCATGACTCAGCA | |
| 376 | CTGACCTTTGAGGGCGAATAGGCACGTGACTTTTCGTTAA | M1 |
| | TATTTACATATGGCGACTTGCTGAGTCATGCTGATGCGTG | |
| | AAATGACT | |
| 377 | CCGAAACCGAAACTATTCAGAAAGCTAATGGATCCGAATT | M1 |
| | GTGTAACCTGATAAACGGTTTAGTCACGTGCCACGCCGAA | |
| | CGTAATGACT | |
| 378 | GCATGACTCAGCATTATCATAAACAGGTGCAAGCCACTAA | M1 |
| | CAATGTGCAGTGTTTCTGTACTGAGTCAGCAATCTCCGGT | |
| | GTCACGTGCC | |
| 379 | TGGAAAATATCCCGAGTATGCACCTGTTTCTGGTGTCCAC | M1 |
| | TTCCGGTCTTTCTACAGTTTCATTATGACTCTTTGTAAAG | |
| | TAAACAAA | |
| 380 | CAGTTTCATTATGACTCAACATAGTTTCGGTTTCGGAGGC | M1 |
| | ATCCACTTCCGGTCTTGTTCGCATTCCATTAGCAGCGTCG | |
| | ACAGGAAGCAGATGTG | |
| 381 | CAGTTTCATTATGACTCTAGGTTAACAGGTGCAATCCCGA | M1 |
| | TCAAAGTCATTAGCGTTCTCGTCAAAGGTCAGTTTCCCGA | |
| | GTTTTTTCCA | |
| 382 | TTTGTTTACTTATTGGTCCTTGCACCTGTTTTGATAGATA | M1 |
| | AGGTGTGAAATGCTTTGACGATTTTCCATGAACAGGTACC | |
| | ACTTCCGGTCT | |
| 383 | AAGGTGTGAAAATCGCAGTAGACCGGAAGTGGAGTCCAAT | M1 |
| | AAAGATAAGAAATTGGTACAGGAAGCAGATGTGTAGTTCT | |
| | CTGACCTTTG | |
| 384 | GCTAATGGATACCGATATTGCTGAGTCATGCATACGACAG | M1 |
| | GAAGCAGATGTGTATCTCCGAAACCGAAACTAACGGGACC | |
| | ACTTCCGGTCT | |
| 385 | TAGTTTCGGTTTCGGTGCGTGTTGCTGACTCAGACGCAAC | M1 |
| | ATCTGACCTTTGTATCTGCAATATGTAAATATTCAGAGAC | |
| | TAAGTAAACAAA | |
| 386 | TGGAAAAAGCTGACTATATATGTAAATATTCGACGTATTG | M1 |
| | CACCTGTTAGGTATTCAAAATAAACAATTTTACTTAGGTA | |
| | ATGACT | |
| 387 | TATGTAAATATAAAATCGTTTTCTTATCTTTTGGTGATCA | M1 |
| | GGAAGCAGATGTGTAGCCATGATTTTCCATTGTAGCTCCT | |
| | CAAAGGTCAG | |
| 388 | TTCTTATCTTTATATCCCTCAGCTAATGGAAGATCGGAAA | M1 |
| | ATATTTACATAACTCCGCTAAACAGGTGCAAGCCAACAAA | |
| | CTGACCTTTG | |
| 389 | CAGGAAGCAGATGTGTCTTCGATATCAGGTTACTCGAACG | M1 |
| | CACATTTTCCAAAGCTCCAATAGTTTCGGTTTCGGTGGTC | |
| | CAATCCATTAGC | |
| 390 | TTTCACACCTTAACTCTCTTTATCAGGTTACAGTTACCCG | M1 |
| | CTAATGACTTGCCTTGAGAAAACAGGTGCATGGCCCACCG | |
| | AAACCGAAACTA | |

Table 3 depicts exemplary enhancer sequences of exemplary macrophage promoters described herein. Table 8 depicts additional exemplary enhancer sequences of exemplary macrophage promoters described herein.

In some embodiments, a promoter or variant as described above may be operably linked to a nucleotide sequence encoding a polypeptide, e.g., an effector molecule described herein.

### Engineered Multicistronic and Multiple Promoter Systems

In some embodiments, engineered nucleic acids are configured to produce multiple effector molecules. For example, nucleic acids may be configured to produce 2-20 different effector molecules. In some embodiments, nucleic acids are configured to produce 2-20, 2-19, 2-18, 2-17, 2-16, 2-15, 2-14, 2-13, 2-12, 2-11, 2-10, 2-9, 2-8, 2-7,2-6, 2-5,2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17,7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 effector molecules. In some embodiments, nucleic acids are configured to produce 1, 2, 3, 4, 5, 6, 7, 8, 9, 10, 11, 12, 13, 14, 15, 16, 17, 18, 19, or 20 effector molecules.

In some embodiments, engineered nucleic acids can be multicistronic, i.e., more than one separate polypeptide (e.g., multiple exogenous polynucleotides or effector molecules) can be produced from a single mRNA transcript. Engineered nucleic acids can be multicistronic through the use of various linkers, e.g., a polynucleotide sequence encoding a first exogenous polynucleotide or effector molecule can be linked to a nucleotide sequence encoding a second exogenous polynucleotide or effector molecule, such as in a first gene:linker:second gene 5′ to 3′ orientation. A linker polynucleotide sequence can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

In some embodiments, when an expression cassette provided herein (e.g., a second expression cassette) comprises two or more units of (L₁-E)x, each L₁ linker polynucleotide sequence is operably associated with the translation of each effector molecule as a separate polypeptide.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide.

In general, a multicistronic system can use any number or combination of linkers, to express any number of genes or portions thereof (e.g., An engineered nucleic acid can encode a first, a second, and a third effector molecule, each separated by linkers such that separate polypeptides encoded by the first, second, and third effector molecules are produced).

“Linkers,” as used herein can refer to polypeptides that link a first polypeptide sequence and a second polypeptide sequence or the multicistronic linkers described above.

### Effector Molecules

Any suitable effector molecule known in the art can be encoded by the engineered nucleic acid or expressed by the engineered cell. Suitable effector molecules can be grouped into therapeutic classes based on structure similarity, sequence similarity, or function. Effector molecule therapeutic classes include, but are not limited to, cytokines, chemokines, homing molecules, growth factors, co-activation molecules, tumor microenvironment modifiers, receptors, ligands, transcription factors, antibodies, polynucleotides, peptides, shRNAs, miRNAs, and enzymes.

In some embodiments, at least one effector molecule or each effector molecule is independently selected from a therapeutic class, wherein the therapeutic class is selected from: a cytokine, a chemokine, a homing molecule, a growth factor, a co-activation molecule, a tumor microenvironment modifier a, a receptor, a ligand, a transcription factor, an antibody, a polynucleotide, a peptide, and an enzyme.

In some embodiments, at least one effector molecule or each effector molecule is independently selected from a therapeutic class, wherein the therapeutic class is selected from: a cytokine, a chemokine, a homing molecule, a growth factor, a co-activation molecule, a tumor microenvironment modifier a, a receptor, a ligand, a transcription factor, an antibody, a peptide, and an enzyme.

In some embodiments, an effector molecule is a transcription factor. The transcription factor may be a master regulator of polarization to an M1 macrophage. Exemplary transcription factor M1 master regulators include, e.g., IRF7, p65/RelA, and derivatives thereof.

In some embodiments, a transcription factor as used in accordance with the present disclosure is IRF7. In some embodiments, an IRF7 comprises an amino acid sequence having at least 70%, at least 80%, at least 90%, at least 95%, at least 96%, at least 97%, at least 98%, at least 99%, or greater sequence identity to SEQ ID NO: 401. In some embodiments, an IRF7 comprises an amino acid sequence as set forth in SEQ ID NO: 401.

In some embodiments, a transcription factor as used in accordance with the present disclosure is p65/RelA. In some embodiments, a p65/RelA comprises an amino acid sequence having at least 70%, at least 80%, at least 90%, at least 95%, at least 96%, at least 97%, at least 98%, at least 99%, or greater sequence identity to SEQ ID NO: 403. In some embodiments, a p65/RelA comprises an amino acid sequence as set forth in SEQ ID NO: 403.

In some embodiments, an effector molecule is a chemokine. Chemokines are small cytokines or signaling proteins secreted by cells that can induce directed chemotaxis in cells. Chemokines can be classified into four main subfamilies: CXC, CC, CX3C and XC, all of which exert biological effects by binding selectively to chemokine receptors located on the surface of target cells. Non-limiting examples of chemokines that may be encoded by the engineered nucleic acids of the present disclosure include: CCL21a, CXCL10, CXCL11, CXCL13, a CXCL10-CXCL11 fusion protein, CCL19, CXCL9, and XCL1, or any combination thereof. In some embodiments, the chemokine is selected from: CCL21a, CXCL10, CXCL11, CXCL13, a CXCL10-CXCL11 fusion protein, CCL19, CXCL9, and XCL1.

In some embodiments, a effector molecule is a cytokine. Non-limiting examples of cytokines that may be encoded by the engineered nucleic acids of the present disclosure include: IL-1alpha, IL1-beta, IL2, IL4, IL6, IL7, IL10, IL13, IL12, an IL12p70 fusion protein, IL12p35m IL12-p40, IL15, IL17A, IL18, IL21, IL22, IL-23, TGF-beta, M-CSF, Type I interferons, Interferon-alpha, GM-CSF, Interferon-gamma, and TNF-alpha, or any combination thereof. In some embodiments, the cytokine is selected from: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, the cytokine is a master regulator of polarization to an M1 macrophage. Exemplary cytokines that are master regulators of M1 polarization include, e.g., IFNgamma, IFNalpha, TNF alpha, GM-CSF, IL-12, IL-12p70, IL-12p40, IL-12p35, IL-6, IL-23, IL-1alpha, IL-1beta, and derivatives thereof.

In some embodiments, the cytokine is a master regulator of polarization to an M2 macrophage. Exemplary cytokines that are master regulators of M2 polarization include, e.g., IL-10, IL-4, IL-13, IL-21, TGF-beta, M-CSF, and derivatives thereof.

In some embodiments, a cytokine as used in accordance with the present disclosure is IFN-gamma. In some embodiments, an IFN-gamma comprises an amino acid sequence having at least 70%, at least 80%, at least 90%, at least 95%, at least 96%, at least 97%, at least 98%, at least 99%, or greater sequence identity to SEQ ID NO: 395. In some embodiments, an IFN-gamma comprises an amino acid sequence as set forth in SEQ ID NO: 395.

In some embodiments, a cytokine as used in accordance with the present disclosure is TNF-alpha. In some embodiments, a TNF-alpha comprises an amino acid sequence having at least 70%, at least 80%, at least 90%, at least 95%, at least 96%, at least 97%, at least 98%, at least 99%, or greater sequence identity to SEQ ID NO: 397. In some embodiments, a TNF-alpha comprises an amino acid sequence as set forth in SEQ ID NO: 397.

In some embodiments, a cytokine as used in accordance with the present disclosure is IL-12p70. In some embodiments, an IL-12p70 comprises an amino acid sequence having at least 70%, at least 80%, at least 90%, at least 95%, at least 96%, at least 97%, at least 98%, at least 99%, or greater sequence identity to SEQ ID NO: 399. In some embodiments, an IL-12p70 comprises an amino acid sequence as set forth in SEQ ID NO: 399.

In some embodiments, a cytokine as used in accordance with the present disclosure is IL-10. In some embodiments, an IL-10 comprises an amino acid sequence having at least 70%, at least 80%, at least 90%, at least 95%, at least 96%, at least 97%, at least 98%, at least 99%, or greater sequence identity to SEQ ID NO: 405. In some embodiments, an IL-10 comprises an amino acid sequence as set forth in SEQ ID NO: 405.

In some embodiments, a cytokine as used in accordance with the present disclosure is IL-4. In some embodiments, an IL-4 comprises an amino acid sequence having at least 70%, at least 80%, at least 90%, at least 95%, at least 96%, at least 97%, at least 98%, at least 99%, or greater sequence identity to SEQ ID NO: 407. In some embodiments, an IL-4 comprises an amino acid sequence as set forth in SEQ ID NO: 407. In some embodiments, an effector molecule (e.g., a cytokine, or any effector molecule described herein) is engineered so that it is tethered to the membrane of a cell. In some embodiments, a tethered effector molecule (e.g., a tethered cytokine) is tethered to the membrane of a cell by operably linking the tethered effector molecule to a transmembrane domain (e.g., a transmembrane domain of a membrane protein).

In some embodiments, effector molecules provided for herein contain a cell-membrane tethering domain (referred to as “MT” in the formula E-L-MT or MT-L-E, where “E” is an effector molecule (e.g., a cytokine) and “L” is a linker, e.g., a linker comprising a cleavable linker). In general, the cell-membrane tethering domain can be any amino acid sequence motif capable of directing a polypeptide of interest (e.g., an effector molecule, e.g., a cytokine) to be localized to (e.g., inserted into), or otherwise associated with, the cell membrane of the cell expressing the polypeptide of interest. The cell-membrane tethering domain can be a transmembrane-intracellular domain. The cell-membrane tethering domain can be a transmembrane domain. The cell-membrane tethering domain can be an integral membrane protein domain (e.g., a transmembrane domain). The cell-membrane tethering domain can be derived from a Type I, Type II, or Type III transmembrane protein. The cell-membrane tethering domain can include post-translational modification tag, or motif capable of post-translational modification to modify the polypeptide of interest to include a post-translational modification tag, where the post-translational modification tag allows association with a cell membrane. Examples of post-translational modification tags include, but are not limited to, lipid-anchor domains (e.g., a GPI lipid-anchor, a myristoylation tag, or palmitoylation tag). Examples of cell-membrane tethering domains include, but are not limited to, a transmembrane-intracellular domain and/or transmembrane domain derived from PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, or BTLA. The cell membrane tethering domain can be a cell surface receptor or a cell membrane-bound portion thereof.

In some embodiments, the cell-membrane tethering domain is or comprises a transmembrane domain of a B7-1 protein, or a functional portion thereof. In some embodiments, the transmembrane domain comprises the sequence

(SEQ ID NO: 481)

LLPSWAITLISVNGIFVICCLTYCFAPRCRERRRNERLRRESVRPV

In some embodiments, the cell-membrane tethering domain comprises a transmembrane domain of a CD8 polypeptide, or a functional portion thereof. Any suitable CD8 polypeptide may be used. Exemplary CD8 polypeptides include, without limitation, NCBI Reference Nos. NP_001139345 and AAA92533.1. Examples of CD8 transmembrane domains include IYIWAPLAGTCGVLLLSLVIT (SEQ ID NO: 485), IYIWAPLAGTCGVLLLSLVITLYCNHR (SEQ ID NO: 486), and IYIWAPLAGTCGVLLLSLVITLYCNHRN (SEQ ID NO: 487). In some embodiments, the transmembrane domain comprises the sequence IYIWAPLAGTCGVLLLSLVIT (SEQ ID NO: 485). In some embodiments, the transmembrane domain comprises the sequence IYIWAPLAGTCGVLLLSLVITLYCNHR (SEQ ID NO: 486). In some embodiments, the transmembrane domain comprises the sequence IYIWAPLAGTCGVLLLSLVITLYCNHRN (SEQ ID NO: 487). In some embodiments, the cell-membrane tethering domain comprises a hinge and transmembrane domain derived from CD8. In some embodiments, the CD8 hinge comprises the sequence

(SEQ ID NO: 488)

TTTPAPRPPTPAPTIALQPLSLRPEACRPAAGGAVHTRGLDFACD

In some embodiments, the CD8 hinge comprises the sequence

(SEQ ID NO: 489)
AAAFVPVFLPAKPTTTPAPRPPTPAPTIASQPLSLRPEACRPAAGGAVHT
RGLDFACDIYIWAPLAGTCGVLLLSLVITLYCNHRN

In some embodiments, the cell membrane tethering domain is either: (1) C-terminal of a linker and N-terminal of any intracellular domain, if present (in other words, the cell membrane tethering domain is in between a linker and, if present, an intracellular domain); or (2) N-terminal of a linker and C-terminal of any intracellular domain, if present (also between the linker and, if present, an intracellular domain with domain orientation inverted). The cell membrane tethering domain can be connected to an intracellular domain, if present, by a polypeptide linker, i.e., a polypeptide sequence not generally considered to be part of the cell membrane tethering domain or the intracellular domain. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 490]), A(EAAAK)₃A (SEQ ID NO: 491), and Whitlow linkers (e.g., a “KEGS” linker such as the amino acid sequence KESGSVSSEQLAQFRSLD (SEQ ID NO: 492), an eGK linker such as the amino acid sequence EGKSSGSGSESKST (SEQ ID NO: 493), and linkers described in more detail in U.S. Pat. No. 5,990,275 herein incorporated by reference). Other polypeptide linkers may be selected based on desired properties (e.g., length, flexibility, amino acid composition etc.) and are known to those skilled in the art.

In some embodiments, a linker is a cleavable linker, e.g., comprising a protease cleavage site. In some embodiments, the cell membrane tethering domain can be connected to a protease cleavage site by a polypeptide linker, i.e., a polypeptide sequence not generally considered to be part of cell membrane tethering domain or protease cleavage site.

In some embodiments, the cell-membrane tethering domain is oriented such that the effector molecule and optionally the protease cleavage site are extracellularly exposed following insertion into, or association with, the cell membrane. In some embodiments wherein the linker comprises a protease cleavage site, the effector molecule and the protease cleavage site are extracellularly exposed such that the protease cleavage site is capable of being cleaved by its respective protease and releasing (“secreting”) the effector molecule into the extracellular space.

In some embodiments a transmembrane domain comprises a B7-1 transmembrane domain. In some embodiments a B7-1 transmembrane domain comprises an amino acid sequence as set forth in SEQ ID NO: 481. In some embodiments, an effector molecule is operably linked to a transmembrane domain via a linker. In some embodiments, a linker comprises an amino acid sequence as set forth in SEQ ID NO: 478.

In some embodiments, a tethered effector molecule is a tethered cytokine. In some embodiments, a tethered cytokine is a tethered IL-10 cytokine. In some embodiments, a tethered IL-10 cytokine comprises an amino acid sequence having at least 70%, at least 80%, at least 90%, at least 95%, at least 96%, at least 97%, at least 98%, at least 99%, or greater sequence identity to SEQ ID NO: 467. In some embodiments, a tethered IL-10 cytokine comprises an amino acid sequence as set forth in SEQ ID NO: 467.

In some embodiments, a tethered cytokine is a tethered IFNg cytokine. In some embodiments, a tethered IFNg cytokine comprises an amino acid sequence having at least 70%, at least 80%, at least 90%, at least 95%, at least 96%, at least 97%, at least 98%, at least 99%, or greater sequence identity to SEQ ID NO: 469. In some embodiments, a tethered IFNg cytokine comprises an amino acid sequence as set forth in SEQ ID NO: 469. In some embodiments, a tethered IFNg cytokine comprises a truncated IFNg. In some embodiments, a truncated IFNg comprises an amino acid sequence as set forth in SEQ ID NO: 477.

In some embodiments, engineered nucleic acids are configured to produce at least one homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. Non-limiting examples of homing molecules include CXCR1, CCR9, CXCR2, CXCR3, CXCR4, CCR2, CCR4, FPR2, VEGFR, IL6R, CXCR1, CSCR7, PDGFR, anti-integrin alpha4,beta7; anti-MAdCAM; CCR9; CXCR4; SDF1; MMP-2; CXCR1; CXCR7; CCR2; CCR4; and GPR15, or any combination thereof. In some embodiments, the homing molecule is selected from: anti-integrin alpha4,beta7; anti-MAdCAM; CCR9; CXCR4; SDF1; MMP-2; CXCR1; CXCR7; CCR2; CCR4; and GPR15.

In some embodiments, engineered nucleic acids are configured to produce at least one growth factor. Suitable growth factors for use as an effector molecule include, but are not limited to, FLT3L and GM-CSF, or any combination thereof. In some embodiments, the growth factor is selected from: FLT3L and GM-CSF.

In some embodiments, engineered nucleic acids are configured to produce at least one co-activation molecule. Suitable co-activation molecules for use as an effector molecule include, but are not limited to, c-Jun, 4-1BBL and CD40L, or any combination thereof. In some embodiments, the co-activation molecule is selected from: c-Jun, 4-1BBL and CD40L.

A “tumor microenvironment” is the cellular environment in which a tumor exists, including surrounding blood vessels, immune cells, fibroblasts, bone marrow-derived inflammatory cells, lymphocytes, signaling molecules and the extracellular matrix (ECM) (see, e.g., Pattabiraman, D. R. & Weinberg, R. A. Nature Reviews Drug Discovery 13, 497-512 (2014); Balkwill, F. R. et al. J Cell Sci 125, 5591-5596, 2012; and Li, H. et al. J Cell Biochem 101(4), 805-15, 2007). Suitable tumor microenvironment modifiers for use as an effector molecule include, but are not limited to, adenosine deaminase, TGFbeta inhibitors, immune checkpoint inhibitors, VEGF inhibitors, and HPGE2, or any combination thereof. In some embodiments, the tumor microenvironment modifier is selected from: adenosine deaminase, TGFbeta inhibitors, immune checkpoint inhibitors, VEGF inhibitors, and HPGE2.

In some embodiments, engineered nucleic acids are configured to produce at least one TGFbeta inhibitor. Suitable TGFbeta inhibitors for use as an effector molecule include, but are not limited to, an anti-TGFbeta peptide, an anti-TGFbeta antibody, a TGFb-TRAP, or combinations thereof. In some embodiments, the TGFbeta inhibitors are selected from: an anti-TGFbeta peptide, an anti-TGFbeta antibody, a TGFb-TRAP, and combinations thereof.

In some embodiments, engineered nucleic acids are configured to produce at least one immune checkpoint inhibitor. Suitable immune checkpoint inhibitors for use as an effector molecule include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies, or any combination thereof. In some embodiments, the immune checkpoint inhibitors are selected from: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies.

Illustrative immune checkpoint inhibitors include pembrolizumab (anti-PD-1; MK-3475/Keytruda®—Merck), nivolumamb (anti-PD-1; Opdivo®—BMS), pidilizumab (anti-PD-1 antibody; CT-011—Teva/CureTech), AMP224 (anti-PD-1; NCI), avelumab (anti-PD-L1; Bavencio®—Pfizer), durvalumab (anti-PD-L1; MEDI4736/Imfinzi®—Medimmune/AstraZeneca), atezolizumab (anti-PD-L1; Tecentriq®-Roche/Genentech), BMS-936559 (anti-PD-L1—BMS), tremelimumab (anti-CTLA-4; Medimmune/AstraZeneca), ipilimumab (anti-CTLA-4; Yervoy®—BMS), lirilumab (anti-KIR; BMS), monalizumab (anti-NKG2A; Innate Pharma/AstraZeneca).

In some embodiments, engineered nucleic acids are configured to produce at least one VEGF inhibitor. Suitable VEGF inhibitors for use as an effector molecule include, but are not limited to, anti-VEGF antibodies, anti-VEGF peptides, or combinations thereof. In some embodiments, the VEGF inhibitors comprise anti-VEGF antibodies, anti-VEGF peptides, or combinations thereof.

In some embodiments, each effector molecule is a human-derived effector molecule.

In some embodiments, an effector molecule is operably linked to a degron domain. The degron domain can be any amino acid sequence motif capable of directing regulated degradation, such as regulated degradation through a ubiquitin-mediated pathway. Exemplary degron domains can include, e.g., a PEST domain, HCV NS4 degron, GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 190) phospho-dependent degron, an Siah binding motif, an SPOP SBC docking motif, a PCNA binding PIP box, and derivatives thereof. Exemplary degron domains are described in International Patent Application Pub. No. WO2022155500A1 and WO2022/266396A1, which are hereby incorporated by reference.

In some embodiments, the degron domain is a PEST domain. PEST domains comprise sequences enriched with Pro, Glu, Ser, and Thr. Exemplary PEST domains are described in Cancer Res., 64 (2004), pp. 8821-8825; Journal of Biological Chemistry 274.43 (1999): 30874-30881; International Patent Application Pub. No. WO2022155500A1; and WO2022/266396A1, which are hereby incorporated by reference.

In some embodiments, the PEST domain comprises the amino acid sequence SEQ ID NO: 501 or a derivative thereof (e.g., a functional fragment thereof). In some embodiments, the derivative of SEQ ID NO: 501 comprises a sequence at least 75%, at least 80%, at least 85%, at least 90%, at least 95%, at least 96%, at least 97%, at least 98%, or at least 99% identical to SEQ ID NO: 501.

In some embodiments, engineered nucleic acids are configured to produce at least one reporter molecule. In some embodiments, a reporter molecule is a fluorescent protein (e.g., luciferase, nanoluciferase, GFP, or variants or derivatives thereof). In some embodiments, a reporter molecule is an EGFP. In some embodiments, an EGFP comprises an amino acid sequence as set forth in SEQ ID NO: 409. In some embodiments, a reporter molecule comprises mCherry. In some embodiments, an mCherry comprises an amino acid sequence as set forth in SEQ ID NO: 411. In some embodiments, a reporter molecule comprises a nanoLuc. In some embodiments, a nanoLuc comprises an amino acid sequence as set forth in SEQ ID NO: 413.

Sequences of exemplary effector molecules and other payloads (e.g., reporter molecules) are in Table 7.

### Secretion Signals

In general, the one or more effector molecules comprise a secretion signal peptide (also referred to as a signal peptide or signal sequence) at the effector molecule's N-terminus that direct newly synthesized proteins destined for secretion or membrane insertion to the proper protein processing pathways. In embodiments with two or more effector molecules, each effector molecule can comprise a secretion signal (S). In embodiments with two or more effector molecules, each effector molecule can comprise a secretion signal such that each effector molecule is secreted from an engineered cell. In some embodiments, an expression cassette (e.g., a second expression cassette) comprising one or more units of (L-E)x further comprises a polynucleotide sequence encoding a secretion signal peptide (S). In some embodiments, for each X the corresponding secretion signal peptide is operably associated with the effector molecule. In some embodiments, the second expression cassette comprising a promoter and a second exogenous polynucleotide sequence having the formula: (L-S-E)x.

The secretion signal peptide operably associated with a effector molecule can be a native secretion signal peptide native secretion signal peptide (e.g., the secretion signal peptide generally endogenously associated with the given effector molecule). The secretion signal peptide operably associated with a effector molecule can be a non-native secretion signal peptide native secretion signal peptide. Non-native secretion signal peptides can promote improved expression and function, such as maintained secretion, in particular environments, such as tumor microenvironments. Non-limiting examples of non-native secretion signal peptide are shown in Table 4.

TABLE 4
Exemplary Signal Secretion Peptides

| | | Source | |
|---|---|---|---|
| Name | Protein SEQUENCE | (Uniprot) | DNA SEQUENCE |
| IL-12 | MCHQQLVISWFSLV | P29460 | ATGTGTCACCAGCAGCTCGTTATA |
| | FLASPLVA (SEQ ID | | TCCTGGTTTAGTTTGGTGTTTCTC |
| | NO: 56) | | GCTTCACCCCTGGTGGCA (SEQ |
| | | | ID NO: 31) |
| IL-12 (Codon | MCHQQLVISWFSLV | | ATGTGCCATCAGCAACTCGTCATC |
| Optimized) | FLASPLVA (SEQ ID | | TCCTGGTTCTCCCTTGTGTTCCTC |
| | NO: 57) | | GCTTCCCCTCTGGTCGCC (SEQ |
| | | | ID NO: 32) |
| IL-2 (Optimized) | MQLLSCIALILALV | | ATGCAACTGCTGTCATGTATCGCA |
| | (SEQ ID NO: 58) | | CTCATCCTGGCGCTGGTA (SEQ |
| | | | ID NO: 33) |
| IL-2 (Native) | MYRMQLLSCIALSL | P60568 | ATGTATCGGATGCAACTTTTGAGC |
| | ALVTNS (SEQ ID | | TGCATCGCATTGTCTCTGGCGCTG |
| | NO: 59) | | GTGACAAATTCC (SEQ ID NO: |
| | | | 34) |
| Trypsinogen-2 | MNLLLILTFVAAAV | P07478 | ATGAATCTCTTGCTCATACTTACG |
| | A (SEQ ID NO: 60) | | TTTGTCGCTGCTGCCGTTGCG |
| | | | (SEQ ID NO: 35) |
| Gaussia | MGVKVLFALICIAV | | ATGGGCGTGAAGGTCTTGTTTGCC |
| Luciferase | AEA (SEQ ID NO: | | CTTATCTGCATAGCTGTTGCGGAG |
| | 61) | | GCG (SEQ ID NO: 36) |
| CD5 | MPMGSLQPLATLY | P06127 | ATGCCGATGGGGAGCCTTCAACCT |
| | LLGMLVASCLG | | TTGGCAACGCTTTATCTTCTGGGG |
| | (SEQ ID NO: 62) | | ATGTTGGTTGCTAGTTGCCTTGGG |
| | | | (SEQ ID NO: 37) |
| IgKVII (mouse) | METDTLLLWVLLL | | ATGGAAACTGACACGTTGTTGCTG |
| | WVPGSTGD (SEQ ID | | TGGGTATTGCTCTTGTGGGTCCCA |
| | NO: 63) | | GGATCTACGGGCGAC (SEQ ID |
| | | | NO: 38) |
| IgKVII (human) | MDMRVPAQLLGLL | P01597 | ATGGATATGAGGGTTCCCGCCCAG |
| | LLWLRGARC (SEQ | | CTTTTGGGGCTGCTTTTGTTGTGG |
| | ID NO: 64) | | CTTCGAGGGGCTCGGTGT (SEQ |
| | | | ID NO: 39) |
| VSV-G | MKCLLYLAFLFIGV | | ATGAAGTGTCTGTTGTACCTGGCG |
| | NC (SEQ ID NO: | | TTTCTGTTCATTGGTGTAAACTGT |
| | 65) | | (SEQ ID NO: 40) |
| Prolactin | MNIKGSPWKGSLLL | P01236 | ATGAATATCAAAGGAAGTCCGTGG |
| | LLVSNLLLCQSVAP | | AAGGGTAGTCTCCTGCTGCTCCTC |
| | (SEQ ID NO: 66) | | GTATCTAACCTTCTCCTTTGTCAA |
| | | | TCCGTGGCACCC (SEQ ID NO: |
| | | | 41) |
| Serum albumin | MKWVTFISLLFLFS | P02768 | ATGAAATGGGTAACATTCATATCA |
| preproprotein | SAYS (SEQ ID NO: | | CTTCTCTTTCTGTTCAGCTCTGCG |
| | 67) | | TATTCT (SEQ ID NO: 42) |
| Azurocidin | MTRLTVLALLAGLL | 20160 | ATGACAAGGCTTACTGTTTTGGCT |
| preproprotein | ASSRA (SEQ ID NO: | | CTCCTCGCTGGACTCTTGGCTTCC |
| | 68) | | TCCCGAGCA (SEQ ID NO: |
| | | | 43) |
| Osteonectin | MRA WIFFLLCLAGR | P09486 | ATGAGGGCTTGGATTTTTTTTCTG |
| (BM40) | ALA (SEQ ID NO: | | CTCTGCCTTGCCGGTCGAGCCCTG |
| | 69) | | GCG (SEQ ID NO: 44) |
| CD33 | MPLLLLLPLLWAG | P20138 | ATGCCTCTTCTGCTTTTGCTTCCT |
| | ALA (SEQ ID NO: | | CTTTTGTGGGCAGGTGCCCTCGCA |
| | 70) | | (SEQ ID NO: 45) |
| IL-6 | MNSFSTSAFGPVAF | P05231 | ATGAACTCTTTCTCAACCTCTGCG |
| | SLGLLLVLPAAFPA | | TTTGGTCCGGTCGCTTTCTCCCTT |
| | P (SEQ ID NO: 71) | | GGGCTCCTGCTTGTCTTGCCAGCA |
| | | | GCGTTTCCTGCGCCA (SEQ ID |
| | | | NO: 46) |
| IL-8 | MTSKLAVALLAAF | P10145 | ATGACAAGTAAACTGGCGGTAGCC |
| | LISAALC (SEQ ID | | TTGCTCGCGGCCTTTTTGATTTCC |
| | NO: 72) | | GCAGCCCTTTGT (SEQ ID NO: |
| | | | 47) |
| CCL2 | MKVSAALLCLLLIA | P13500 | ATGAAGGTAAGTGCAGCGTTGCTT |
| | ATFIPQGLA (SEQ ID | | TGCCTTCTCCTCATTGCAGCGACC |
| | NO: 73) | | TTTATTCCTCAAGGGCTGGCC |
| | | | (SEQ ID NO: 48) |
| TIMP2 | MGAAARTLRLALG | P16035 | ATGGGAGCGGCAGCTAGAACACTT |
| | LLLLATLLRPADA | | CGACTTGCCCTTGGGCTCTTGCTC |
| | (SEQ ID NO: 74) | | CTTGCAACCCTCCTTAGACCTGCC |
| | | | GACGCA (SEQ ID NO: 49) |
| VEGFB | MSPLLRRLLLAALL | P49765 | ATGTCACCGTTGTTGCGGAGATTG |
| | QLAPAQA (SEQ ID | | CTGTTGGCCGCACTTTTGCAACTG |
| | NO: 75) | | GCTCCTGCTCAAGCC (SEQ ID |
| | | | NO: 50) |
| Osteoprotegerin | MNNLLCCALVFLDI | O00300 | ATGAATAACCTGCTCTGTTGTGCG |
| | SIKWTTQ (SEQ ID | | CTCGTGTTCCTGGACATTTCTATA |
| | NO: 76) | | AAATGGACAACGCAA (SEQ ID |
| | | | NO: 51) |
| Serpin E1 | MQMSPALTCLVLG | P05121 | ATGCAAATGTCTCCTGCCCTTACC |
| | LALVFGEGSA (SEQ | | TGTCTCGTACTTGGTCTTGCGCTC |
| | ID NO: 77) | | GTATTTGGAGAGGGATCAGCC |
| | | | (SEQ ID NO: 52) |
| GROalpha | MARAALSAAPSNP | P09341 | ATGGCAAGGGCTGCACTCAGTGCT |
| | RLLRVALLLLLLVA | | GCCCCGTCTAATCCCAGATTGCTT |
| | AGRRAAG (SEQ ID | | CGAGTTGCATTGCTTCTTCTGTTG |
| | NO: 78) | | CTGGTTGCAGCTGGTAGGAGAGCA |
| | | | GCGGGT (SEQ ID NO: 53) |
| CXCL12 | MNAKVVVVLVLVL | P48061 | ATGAATGCAAAAGTCGTGGTCGTG |
| | TALCLSDG (SEQ ID | | CTGGTTTTGGTTCTGACGGCGTTG |
| | NO: 79) | | TGTCTTAGTGATGGG (SEQ ID |
| | | | NO: 54) |
| IL-21 (Codon | MERIVICLMVIFLGT | Q9HBE4 | ATGGAACGCATTGTGATCTGCCTG |
| Optimized) | LVHKSSS (SEQ ID | | ATGGTCATCTTCCTGGGCACCTTA |
| | NO: 80) | | GTGCACAAGTCGAGCAGC (SEQ |
| | | | ID NO: 55) |
| CD8a | MALPVTALLLPLAL | P01732 | ATGGCTCTCCCTGTAACTGCCCTG |
| | LLHAARP | | CTTCTTCCCCTTGCCTTGCTTCTC |
| | (SEQ ID NO: 83) | | CATGCCGCTAGACCC (SEQ ID |
| | | | NO: 84) |
| GMCSFR | MLLLVTSLLLCELP | P15509 | ATGCTGCTGCTGGTCACATCTCTG |
| | HPAFLLIP | | CTGCTGTGCGAGCTGCCCCATCCT |
| | (SEQ ID NO: 85) | | GCCTTTCTGCTGATCCCT (SEQ |
| | | | ID NO: 86) |
| | | | ATGCTGCTGCTGGTTACATCTCTG |
| | | | CTGCTGTGCGAGCTGCCCCATCCT |
| | | | GCCTTTCTGCTGATCCCT (SEQ |
| | | | ID NO: 87) |

### Antigen Recognizing Receptors

Certain aspects of the present disclosure relate to an engineered nucleic acid (e.g., any engineered nucleic acid described herein) comprising a polynucleotide sequence encoding an antigen recognizing receptor. In some embodiments, an engineered nucleic acid of the present disclosure comprises a first expression cassette that comprises an antigen recognizing receptor. In some embodiments, the first expression cassette comprises a polynucleotide sequence encoding the antigen recognizing receptor that is operably linked to the first promoter. Suitable antigen recognizing receptors for use as an effector molecule recognize antigens that include, but are not limited to, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin (MSLN), MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1, or any combination thereof.

In some embodiments, the antigen recognizing receptor recognizes an antigen selected from: 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.

In some embodiments, the antigen recognizing receptor comprises an antigen-binding domain. In some embodiments, the antigen-binding domain comprises an antibody, an antigen-binding fragment of an antibody, a F(ab) fragment, a F(ab′) fragment, a single chain variable fragment (scFv), or a single-domain antibody (sdAb). In some embodiments, the antigen-binding domain comprises a single chain variable fragment (scFv). In some embodiments, the scFv comprises a heavy chain variable domain (VH) and a light chain variable domain (VL). In some embodiments, the VH and VL are separated by a peptide linker.

An scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain. In some embodiments, the scFv comprises the structure VH-L-VL or VL-L-VH, wherein VH is the heavy chain variable domain, L is the peptide linker, and VL is the light chain variable domain.

An sdAb is a molecule in which one variable domain of an antibody specifically binds to an antigen without the presence of the other variable domain.

A F(ab) fragment contains the constant domain (CL) of the light chain and the first constant domain (CH1) of the heavy chain along with the variable domains VL and VH on the light and heavy chains respectively. F(ab′) fragments differ from Fab fragments by the addition of a few residues at the carboxy terminus of the heavy chain CH1 domain including one or more cysteines from the antibody hinge region. F(ab′)₂ fragments contain two Fab′ fragments joined, near the hinge region, by disulfide bonds.

In some embodiments, the antigen recognizing receptor is a chimeric antigen receptor (CAR) or T cell receptor (TCR). In some embodiments, the antigen recognizing receptor is a CAR. In some embodiments, the CAR comprises one or more intracellular signaling domains, and the one or more intracellular signaling domains are selected from: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, and a MyD88 intracellular signaling domain. In some embodiments, the CAR comprises a CD3zeta-chain intracellular signaling domain and one or more additional intracellular signaling domains (e.g., co-stimulatory domains) selected from a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

In some embodiments, the CAR further comprises a transmembrane domain, and the transmembrane domain is selected from: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceR1g transmembrane domain, and an NKG2D transmembrane domain.

In some embodiments, the CAR further comprises a spacer region (e.g., hinge domain) between the antigen-binding domain and the transmembrane domain. A spacer or hinge domain is any oligopeptide or polypeptide that functions to link the transmembrane domain to the extracellular domain and/or the intracellular signaling domain in the polypeptide chain. Spacer or hinge domains provide flexibility to the inhibitory chimeric receptor or tumor-targeting chimeric receptor, or domains thereof, or prevent steric hindrance of the inhibitory chimeric receptor or tumor-targeting chimeric receptor, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of an inhibitory chimeric receptor or tumor-targeting chimeric receptor.

Exemplary spacer or hinge domains may include, without limitation an IgG domain (such as an IgG1 hinge, an IgG2 hinge, an IgG3 hinge, or an IgG4 hinge), an IgD hinge domain, a CD8a hinge domain, and a CD28 hinge domain. In some embodiments, the spacer or hinge domain is an IgG domain, an IgD domain, a CD8a hinge domain, or a CD28 hinge domain.

Exemplary spacer or hinge domain protein sequences are shown in Table 5. Exemplary spacer or hinge domain nucleotide sequences are shown in Table 6.

TABLE 5

| | SEQ ID | |
|---|---|---|
| Amino Acid Sequence | NO: | Description |
| AAAIEVMYPPPYLDNEKSNGTIIHVKGKHLCPSPLF | 100 | CD28 hinge |
| PGPSKP | | |
| ESKYGPPCPSCP | 101 | IgG4 minimal hinge |
| ESKYGPPAPSAP | 102 | IgG4 minimal hinge, no disulfides |
| ESKYGPPCPPCP | 103 | IgG4 S228P minimal hinge, enhanced |
| | | disulfide formation |
| EPKSCDKTHTCP | 104 | IgG1 minimal hinge |
| AAAFVPVFLPAKPTTTPAPRPPTPAPTIASQPLSLR | 105 | Extended CD8a hinge |
| PEACRPAAGGAVHTRGLDFACDIYIWAPLAGTCGVL | | |
| LLSLVITLYCNHRN | | |
| TTTPAPRPPTPAPTIALQPLSLRPEACRPAAGGAVH | 106 | CD8a hinge |
| TRGLDFACD | | |
| ACPTGLYTHSGECCKACNLGEGVAQPCGANQTVCEP | 107 | LNGFR hinge |
| CLDSVTFSDVVSATEPCKPCTECVGLQSMSAPCVEA | | |
| DDAVCRCAYGYYQDETTGRCEACRVCEAGSGLVFSC | | |
| QDKQNTVCEECPDGTYSDEADAEC | | |
| ACPTGLYTHSGECCKACNLGEGVAQPCGANQTVC | 108 | Truncated LNGFR hinge (TNFR- |
| | | Cys1) |
| AVGODTQEVIVVPHSLPFKV | 109 | PDGFR-beta extracellular linker |

TABLE 6

| Nucleic Acid Sequence | SEQ ID NO: | Description |
|---|---|---|
| GCAGCAGCTATCGAGGTGATGTATCCTCCGC | 110 | CD28 hinge |
| CCTACCTGGATAATGAAAAGAGTAATGGGA | | |
| CTATCATTCATGTAAAAGGGAAGCATCTTTG | | |
| TCCTTCTCCCCTTTTCCCCGGTCCGTCTAAAC | | |
| CT | | |
| GAA AGC AAG TAC GGT CCA CCT TGC CCT | 111 | IgG4 minimal hinge |
| AGC TGT CCG | | |
| GAA TCC AAG TAC GGC CCC CCA GCG CCT | 112 | IgG4 minimal hinge, no |
| AGT GCC CCA | | disulfides |
| GAA TCT AAA TAT GGC CCG CCA TGC CCG | 113 | IgG4 S228P minimal hinge, |
| CCT TGC CCA | | enhanced disulfide formation |
| GAA CCG AAG TCT TGT GAT AAA ACT CAT | 114 | IgG1 minimal hinge |
| ACG TGC CCG | | |
| GCT GCT GCT TTC GTA CCC GTG TTC CTC | 115 | Extended CD8a hinge |
| CCT GCT AAG CCT ACG ACT ACC CCC GCA | | |
| CCG AGA CCA CCC ACG CCA GCA CCC ACG | | |
| ATTGCT AGC CAG CCC CTT AGT TTG CGA | | |
| CCA GAA GCT TGT CGG CCT GCT GCT GGT | | |
| GGC GCG GTA CAT ACC CGC GGC CTT GAT | | |
| TTT GCTTGC GAT ATA TAT ATC TGG GCG | | |
| CCT CTG GCC GGA ACA TGC GGG GTC CTC | | |
| CTC CTT TCT CTG GTT ATT ACT CTC TAC | | |
| TGT AAT CACAGG AAT | | |
| GCC TGC CCG ACC GGG CTC TAC ACT CAT | 116 | LNGFR hinge |
| AGC GGG GAA TGT TGT AAG GCA TGT AAC | | |
| TTG GGT GAG GGC GTC GCA CAG CCC TGC | | |
| GGAGCT AAC CAA ACA GTG TGC GAA CCC | | |
| TGC CTC GAT AGT GTG ACG TTC TCT GAT | | |
| GTT GTA TCA GCT ACA GAG CCT TGC AAA | | |
| CCA TGTACT GAG TGC GTT GGA CTT CAG | | |
| TCA ATG AGC GCT CCA TGT GTG GAG GCA | | |
| GAT GAT GCG GTC TGT CGA TGT GCT TAC | | |
| GGA TAC TACCAA GAC GAG ACA ACA GGG | | |
| CGG TGC GAG GCC TGT AGA GTT TGT GAG | | |
| GCG GGC TCC GGG CTG GTG TTT TCA TGT | | |
| CAA GAC AAG CAAAAT ACG GTC TGT GAA | | |
| GAG TGC CCT GAT GGC ACC TAC TCA GAC | | |
| GAA GCA GAT GCA GAA TGC | | |
| GCC TGC CCT ACA GGA CTC TAC ACG CAT | 117 | Truncated LNGFR hinge (TNFR- |
| AGC GGT GAG TGT TGT AAA GCA TGC AAC | | Cys1) |
| CTC GGG GAA GGT GTA GCC CAG CCA TGC | | |
| GGG GCT AAC CAA ACC GTT TGC | | |
| GCTGTGGGCCAGGACACGCAGGAGGTCATC | 118 | PDGFR-beta extracellular |
| GTGGTGCCACACTCCTTGCCCTTTAAGGTG | | linker |

Suitable transmembrane domains, spacer or hinge domains, and intracellular domains for use in a CAR are generally described in Stoiber et al, Cells 2019, 8(5), 472; Guedan et al, Mol Therapy: Met & Clinic Dev, 2019 12:145-156; and Sadelain et al, Cancer Discov; 2013, 3(4); 388-98, each of which are hereby incorporated by reference in their entirety.

In some embodiments, the CAR further comprises a secretion signal peptide. Any suitable secretion signal peptide of the present disclosure may be used.

### Post-Transcriptional Regulatory Elements

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element.

### Immunoresponsive Cells

Certain aspects of the present disclosure relate to a cell, such as an immunoresponsive cell, that has been genetically engineered to comprise one or more nucleic acids of the present disclosure, and to methods of using such cells for treating solid tumors.

In some embodiments, the cell is a mammalian cell. In some embodiments, the mammalian cell is a primary cell. In some embodiments, the mammalian cell is a cell line. In some embodiments, the mammalian cell a bone marrow cell, a blood cell, a skin cell, bone cell, a muscle cell, a neuronal cell, a fat cell, a liver cell, or a heart cell. In some embodiments, the cell is a stem cell. Exemplary stem cells include, without limitation embryonic stem cells (ESCs), induced pluripotent stem cells (iPSCs), adult stem cells, and tissue-specific stem cells, such as hematopoietic stem cells (blood stem cells), mesenchymal stem cells (MSC), neural stem cells, epithelial stem cells, or skin stem cells. In some embodiments, the cell is a cell that is derived or differentiated from a stem cell of the present disclosure. In some embodiments, the cell is an immune cell. Immune cells of the present disclosure may be isolated or differentiated from a stem cell of the present disclosure (e.g., from an ESC or iPSC). Exemplary immune cells include, without limitation, T cells (e.g., helper T cells, cytotoxic T cells, memory T cells, regulatory T cells, natural killer T cells, alpha beta T cells, and gamma delta T cells), B cells, natural killer (NK) cells, dendritic cells, myeloid cells, macrophages, and monocytes. In some embodiments, the cell is a neuronal cell. Neuronal cells of the present disclosure may be isolated or differentiated from a stem cell of the present disclosure (e.g., from an ESC or iPSC). Exemplary neuronal cells include, without limitation, neural progenitor cells, neurons (e.g., sensory neurons, motor neurons, cholinergic neurons, GABAergic neurons, glutamatergic neurons, dopaminergic neurons, or serotonergic neurons), astrocytes, oligodendrocytes, and microglia.

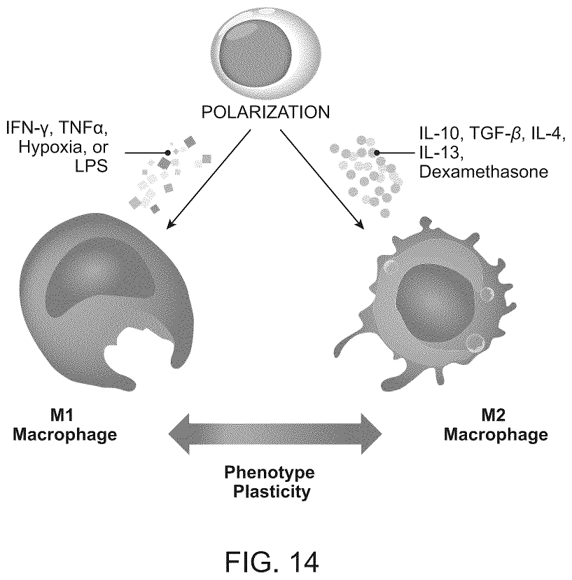

In some embodiments, the cell is an immunoresponsive cell. Immunoresponsive cells of the present disclosure may be isolated or differentiated from a stem cell of the present disclosure (e.g., from an ESC or iPSC). Exemplary immunoresponsive cells of the present disclosure include, without limitation, cells of the lymphoid lineage. The lymphoid lineage, comprising B cells, T cells, and natural killer (NK) cells, provides for the production of antibodies, regulation of the cellular immune system, detection of foreign agents in the blood, detection of cells foreign to the host, and the like. Examples of immunoresponsive cells of the lymphoid lineage include, without limitation, T cells, Natural Killer (NK) cells, embryonic stem cells, pluripotent stem cells, and induced pluripotent stem cells (e.g., those from which lymphoid cells may be derived or differentiated). Macrophages are white blood cell that phagocytose and degrade cellular debris, foreign substances, microbes, cancer cells, etc. In addition to their role in phagocytosis, these cells play an important role in development, tissue maintenance and repair, and in both innate and adaptive immunity in that they recruit and influence other cells including immune cells such as lymphocytes. Macrophages can exist in many phenotypes, including phenotypes that have been referred to as M1 and M2. Macrophages that perform primarily pro-inflammatory functions are called M1 macrophages (i.e., CD86⁺/CD68⁺), whereas macrophages that decrease inflammation and encourage and regulate tissue repair are called M2 macrophages (i.e., CD206⁺/CD68⁺). Engineering of macrophages is described, e.g., in WO2017044487, Brempelis K J et al. J Immunother Cancer. 2020; 8(2):e001356, and Xia et al., Adv. Mater. 2020, 32, 2002054. Macrophages can be polarized to M1 or M2 states by various extracellular cues. For example, when encountering inflammatory cues such as LPS, TNFα or IFNγ, macrophages can be polarized to a M1 state. Alternatively, when encountering anti-inflammatory cues such as IL-4, TGF-β,IL-10, or dexamethasone, macrophages can be polarized to a M2 state. These polarization phenotypes can be plastic depending on what the cell encounters, e.g., can transition between polarization states depending on the surrounding microenvironment. The plasticity of macrophage polarization state can lead to undesired loss of macrophage activity in vivo when the cells encounter an opposing cue. For example, an M1-polarized cell that is phagocytic may lose its inflammatory or phagocytic ability in the presence of anti-inflammatory cytokines such as IL-4, TGF-β or IL-10. See FIG. 14. This plasticity can be undesirable when engineered macrophages are being used as a cell therapy with either inflammatory or anti-inflammatory activity. There is a need for technologies that enable the synthetic control of macrophage polarization logic.

In the instant disclosure, it is contemplated that M1 and/or M2 phenotype may be “locked” on or undergo a phenotype switch in a manner that is controlled by a state-specific specific promoter. Such lock would prevent the macrophage plasticity and result in regulated expression of the target macrophage activity.

Macrophage polarization state-specific promoters provided herein are useful for implementing macrophage polarization logic, e.g., in a macrophage state-selective manner. Engineered macrophage-specific promoter systems described herein can beneficially provide synthetic macrophage polarization logic, for example, by keeping macrophages in a desired phenotype state (“phenotype lock”) or driving macrophages to switch from an undesired phenotype state to a desired phenotype state (“phenotype switch”).

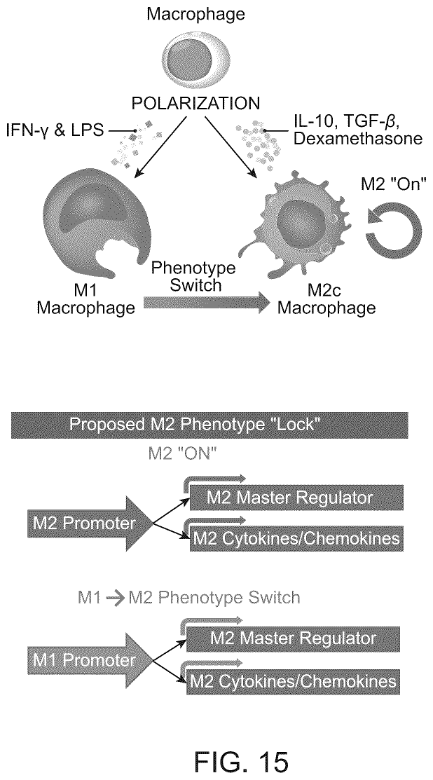

For example, a promoter system can include a promoter having greater activity in an M2 macrophage as compared to an M1 or M0 macrophage (also referred to herein as an M2 promoter, an M2-specifc promoter), operably linked to a polynucleotide encoding an effector molecule that acts as an M2 master regulator, e.g., an effector molecule that controls macrophage polarization state by directing macrophages to an M2 state. The M2 master regulator can be, e.g., an M2 transcription factor or M2 cytokine. Without wishing to be bound by theory, such a promoter system can be used to keep M2 macrophages in a stable M2 state (“M2 Phenotype Lock”), e.g., even in M2 macrophages exposed to opposing cues from the environment. See FIG. 15

For other example, a promoter system can include a promoter having greater activity in an M1 macrophage as compared to an M2 or M0 macrophage (also referred to herein as an M1 promoter, an M1-specifc promoter), operably linked to a polynucleotide encoding an effector molecule that act as an M2 master regulator, e.g., an M2 transcription factor or M2 cytokine. Without wishing to be bound by theory, such a promoter system can be used to direct M1 macrophages from an M1 phenotype to a M2 phenotype (“M1 to M2 Phenotype switch”). See FIG. 15.

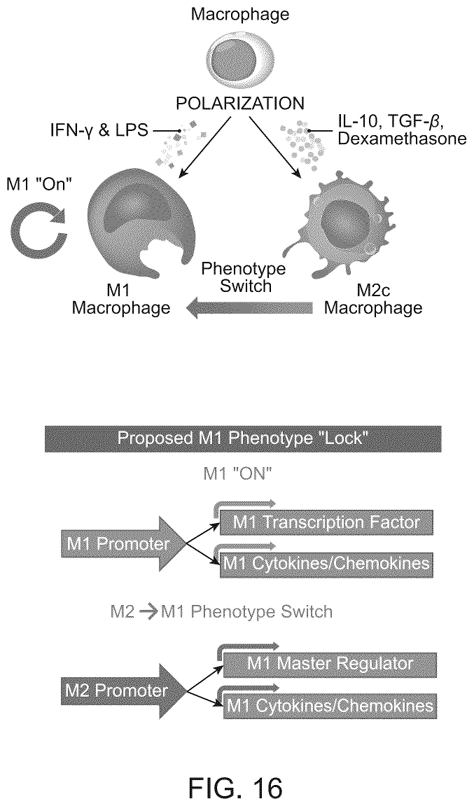

For other example, a promoter system can include a promoter having greater activity in an M1 macrophage as compared to an M2 or M0 macrophage (also referred to herein as an M1 promoter, an M1-specifc promoter), operably linked to a polynucleotide encoding an effector molecule that act as an M1 master regulator e.g., an effector molecule that controls macrophage polarization state by directing macrophages to an M1 state. The M1 master regulator can be, e.g., an M1 transcription factor or M1 cytokine. Without wishing to be bound by theory, such a promoter system can be used to keep M1 macrophages in a stable M1 state (“M1 Phenotype Lock”), e.g., even in M1 macrophages exposed to opposing cues from the environment. See FIG. 16

For other example, a promoter system can include a promoter having greater activity in an M2 macrophage as compared to an M1 or M0 macrophage (also referred to herein as an M2 promoter, an M2-specifc promoter), operably linked to a polynucleotide encoding an effector molecule that act as an M1 master regulator, e.g., an M1 transcription factor or M1 cytokine. Without wishing to be bound by theory, such a promoter system can be used to direct M2 macrophages from an M2 phenotype to a M1 phenotype (“M2 to M1 Phenotype switch”). See FIG. 16.

T cells can be lymphocytes that mature in the thymus and are chiefly responsible for cell-mediated immunity. T cells are involved in the adaptive immune system. In some embodiments, T cells of the present disclosure can be any type of T cells, including, without limitation, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells), natural killer T cells, mucosal associated invariant T cells, and T6 T cells. Cytotoxic T cells (CTL or killer T cells) are a subset of T lymphocytes capable of inducing the death of infected somatic or tumor cells. A patient's own T cells may be genetically modified to target specific antigens through the introduction of one or more chimeric receptors, such as a chimeric TCRs or CARs.

Natural killer (NK) cells can be lymphocytes that are part of cell-mediated immunity and act during the innate immune response. NK cells do not require prior activation in order to perform their cytotoxic effect on target cells.

In some embodiments, an immunoresponsive cell of the present disclosure is a T cell. T cells of the present disclosure may be autologous, allogeneic, or derived in vitro from engineered progenitor or stem cells.

In some embodiments, an immunoresponsive cell of the present disclosure is a universal T cell with deficient TCR-ap. Methods of developing universal T cells are described in the art, for example, in Valton et al., Molecular Therapy (2015); 23 9, 1507-1518, and Torikai et al., Blood 2012 119:5697-5705.

In some embodiments, an immunoresponsive cell of the present disclosure is an isolated immunoresponsive cell comprising one or more chimeric receptors of the present disclosure. In some embodiments, the immunoresponsive cell comprises one or more, two or more, three or more, four or more, five or more, six or more, seven or more, eight or more, nine or more, or ten or more chimeric receptors of the present disclosure.

In some embodiments, an immunoresponsive cell is a T cell. In some embodiments, an immunoresponsive cell is a Natural Killer (NK) cell. In some embodiments, an immunoresponsive cell is a macrophage. In some embodiments, an immunoresponsive cell is an M1 macrophage. In some embodiments, an immunoresponsive cell is an M2 macrophage. In some embodiments, the M2 macrophage is selected from the group consisting of M2a, M2b, and M2c subtypes.

In some embodiments, an immunoresponsive cell expresses or is capable of expressing an immune receptor. Immune receptors generally are capable of inducing signal transduction or changes in protein expression in the immune receptor-expressing cell that results in the modulation of an immune response upon binding to a cognate ligand (e.g., regulate, activate, initiate, stimulate, increase, prevent, attenuate, inhibit, reduce, decrease, inhibit, or suppress an immune response). For example, when CD3 chains present in a TCR/CAR cluster in response to ligand binding, an immunoreceptor tyrosine-based activation motifs (ITAMs)-meditated signal transduction cascade is produced. Specifically, in certain embodiments, when an endogenous TCR, exogenous TCR, chimeric TCR, or a CAR (specifically an activating CAR) binds their respective antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated that in turn can initiate a T cell activation pathway and ultimately activates transcription factors, such as NF-κB and AP-1. These transcription factors are capable of inducing global gene expression of the T cell to increase IL-2 production for proliferation and expression of master regulator T cell proteins in order to initiate a T cell mediated immune response, such as cytokine production and/or T cell mediated killing.

### Cells Expressing Chimeric Antigen Receptors

In some embodiments, a cell of the present disclosure (e.g., an immunoresponsive cell) comprises two or more chimeric receptors. In some embodiments, the cell comprises two or more chimeric receptors, wherein a first of the two or more chimeric receptors is an activating chimeric receptor and a second of the two or more chimeric receptors is a chimeric inhibitory receptor. In some embodiments, the cell comprises a first activating chimeric receptor and a second activating chimeric receptor. In some embodiments, the cell comprises three or more chimeric receptors, wherein at least one of the three or more chimeric receptors is an activating chimeric receptor. In some embodiments, the cell comprises three or more chimeric receptors, wherein at least one of the three or more chimeric receptors is a chimeric inhibitory receptor. In some embodiments, the cell comprises four or more chimeric receptors. In some embodiments, the cell comprises five or more chimeric receptors.

In some embodiments, each of the two or more chimeric receptors comprise a different antigen-binding domain, e.g., that binds to the same antigen or to a different antigen. In some embodiments each antigen bound by the two or more chimeric receptors are expressed on the same cell, such as an epithelial cell type (e.g., same epithelial cell type).

In embodiments where a cell of the present disclosure (e.g., an immunoresponsive cell) expresses two or more distinct chimeric receptors, the antigen-binding domain of each of the different chimeric receptors may be designed such that the antigen-binding domains do not interact with one another. For example, a cell of the present disclosure (e.g., an immunoresponsive cell) expressing a first chimeric receptor and a second chimeric receptor may comprise a first chimeric receptor that comprises an antigen-binding domain that does not form an association with the antigen-binding domain of the second chimeric receptor. For example, the antigen-binding domain of the first chimeric receptor may comprise an antibody fragment, such as an scFv, while the antigen-binding domain of the second chimeric receptor may comprise a V_{H}H.

Without wishing to be bound by theory, it is believed that in cells having a plurality of chimeric membrane embedded receptors that each comprise an antigen-binding domain, interactions between the antigen-binding domains of each of the receptors can be undesirable, because such interactions may inhibit the ability of one or more of the antigen-binding domains to bind their cognate antigens. Accordingly, in embodiments where cells of the present disclosure (e.g., immunoresponsive cells) express two or more chimeric receptors, the chimeric receptors comprise antigen-binding domains that minimize such inhibitory interactions. In one embodiment, the antigen-binding domain of one chimeric receptor comprises an scFv and the antigen-binding domain of the second chimeric receptor comprises a single VH domain, e.g., a camelid, shark, or lamprey single VH domain, or a single VH domain derived from a human or mouse sequence.

In some embodiments, when present on the surface of a cell, binding of the antigen-binding domain of the first chimeric receptor to its cognate antigen is not substantially reduced by the presence of the second chimeric receptor. In some embodiments, binding of the antigen-binding domain of the first chimeric receptor to its cognate antigen in the presence of the second chimeric receptor is 85%, 90%, 95%, 96%, 97%, 98%, or 99% of binding of the antigen-binding domain of the first chimeric receptor to its cognate antigen in the absence of the second chimeric receptor. In some embodiments, when present on the surface of a cell, the antigen-binding domains of the first chimeric receptor and the second chimeric receptor associate with one another less than if both were scFv antigen-binding domains. In some embodiments, the antigen-binding domains of the first chimeric receptor and the second chimeric receptor associate with one another 85%, 90%, 95%, 96%, 97%, 98%, or 99% less than if both were scFv antigen-binding domains.

### Co-Stimulatory Ligands

In some embodiments, a cell of the present disclosure (e.g., an immunoresponsive cell) can further include one or more recombinant or exogenous co-stimulatory ligands. For example, the cell can be further transduced with one or more co-stimulatory ligands, such that the cell co-expresses or is induced to co-express one or more chimeric receptors and one or more co-stimulatory ligands. Without wishing to be bound by theory, it is believed that the interaction between the one or more chimeric receptors and the one or more co-stimulatory ligands may provide a non-antigen-specific signal important for full activation of the cell. Examples of suitable co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Examples of suitable TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta (LTP), CD257/B cell-activating factor (B AFF)/Bly s/THANK/Tall-1, glucocorticoid-induced TNF Receptor ligand (GITRL), and TNF-related apoptosis-inducing ligand (TRAIL), LIGHT (TNFSF 14). The immunoglobulin (Ig) superfamily is a large group of cell surface and soluble proteins that are involved in the recognition, binding, or adhesion processes of cells. These proteins share structural features with immunoglobulins and possess an immunoglobulin domain (fold). Examples of suitable immunoglobulin superfamily ligands include, without limitation, CD80 and CD86, both ligands for CD28, PD-L1/(B7-H1) that are ligands for PD-1. In certain embodiments, the one or more co-stimulatory ligands are selected from 4-1BBL, CD80, CD86, CD70, OX40L, CD48, TNFRSF14, PD-L1, and combinations thereof.

In some embodiments, a cell of the present disclosure (e.g., an immunoresponsive cell) comprises one or more recombinant or exogenous co-stimulatory ligands regulated by an engineered promoter described herein, e.g., an engineered macrophage-specific promoter from Table 1. In certain embodiments, a TNF superfamily member (e.g., nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta (LTP), CD257/B cell-activating factor (B AFF)/Bly s/THANK/Tall-1, glucocorticoid-induced TNF Receptor ligand (GITRL), and TNF-related apoptosis-inducing ligand (TRAIL), LIGHT (TNFSF 14)) is regulated by an engineered promoter described herein, e.g., an engineered macrophage-specific promoter from Table 1. In certain embodiments, an Ig superfamily member ligand (e.g., CD80, CD86, PD-L1, and B7-H1) is regulated by an engineered promoter described herein, e.g., an engineered macrophage-specific promoter from Table 1. In certain embodiments, a co-stimulatory ligand (e.g., 4-1BBL, CD80, CD86, CD70, OX40L, CD48, TNFRSF14, PD-L1, and combinations thereof) is regulated by an engineered promoter described herein, e.g., an engineered promoter from Table 1.

### Chemokine Receptors

In some embodiments, a cell of the present disclosure (e.g., an immunoresponsive cell) comprises one or more chimeric receptors and may further include one or more chemokine receptors. For example, transgenic expression of chemokine receptor CCR2b or CXCR2 in cells, such as T cells, enhances trafficking to CCL2-secreting or CXCL1-secreting solid tumors (Craddock et al, J Immunother. 2010 October; 33(8):780-8 and Kershaw et al. Hum Gene Ther. 2002 Nov. 1; 13(16): 1971-80). Without wishing to be bound by theory, it is believed that chemokine receptors expressed on chimeric receptor-expressing cells of the present disclosure may recognize chemokines secreted by tumors and improve targeting of the cell to the tumor, which may facilitate the infiltration of the cell to the tumor and enhance the antitumor efficacy of the cell. Chemokine receptors of the present disclosure may include a naturally occurring chemokine receptor, a recombinant chemokine receptor, or a chemokine-binding fragment thereof. Examples of suitable chemokine receptors that may expressed on a cell of the present disclosure include, without limitation, a CXC chemokine receptor, such as CXCR1, CXCR2, CXCR3, CXCR4, CXCR5, CXCR6, or CXCR7; a CC chemokine receptor, such as CCR1, CCR2, CCR3, CCR4, CCR5, CCR6, CCR7, CCR8, CCR9, CCR10, or CCR11; a CX3C chemokine receptor, such as CX3CR1; an XC chemokine receptor, such as XCR1; and chemokine-binding fragments thereof. In some embodiments, the chemokine receptor to be expressed on the cell is chosen based on the chemokines secreted by the tumor.

In some embodiments, a cell of the present disclosure (e.g., an immunoresponsive cell) comprises one or more chemokine receptors regulated by an engineered promoter described herein, e.g., a engineered promoter from Table 1. Examples of such chemokine receptors include, for example and without limitation, a CXC chemokine receptor, such as CXCR1, CXCR2, CXCR3, CXCR4, CXCR5, CXCR6, or CXCR7; a CC chemokine receptor, such as CCR1, CCR2, CCR3, CCR4, CCR5, CCR6, CCR7, CCR8, CCR9, CCR10, or CCR11; a CX3C chemokine receptor, such as CX3CR1; an XC chemokine receptor, such as XCR1; and chemokine-binding fragments thereof. In certain embodiments, the chemokine receptor regulated by the engineered promoter described herein, e.g., a engineered promoter from Table 1, is chosen based on the chemokines secreted by the tumor.

### Chimeric Receptor Regulation

Some embodiments of the present disclosure relate to regulating one or more chimeric receptor activities of chimeric receptor-expressing cells of the present disclosure. There are several ways chimeric receptor activities can be regulated. In some embodiments, a regulatable chimeric receptor, wherein one or more chimeric receptor activities can be controlled, may be desirable to optimize the safety and/or efficacy of the chimeric receptor therapy. For example, inducing apoptosis using a caspase fused to a dimerization domain (see, e.g., Di et al., N Engl. J. Med. 2011 Nov. 3; 365(18): 1673-1683) can be used as a safety switch in the chimeric receptor therapy. In some embodiments, a chimeric receptor-expressing cell of the present disclosure can also express an inducible Caspase-9 (iCaspase-9) that, upon administration of a dimerizer drug, such as rimiducid (IUPAC name: [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl)butanoyl]piperidine-2-carbonyl]oxypropyl]phenoxy]acetyl]amino]ethylamino]-2-oxoethoxy]phenyl]propyl](2S)-1-[(2S)-2-(3,4,5-trimethoxyphenyl)butanoyl]piperidine-2-carboxylate), induces activation of the Caspase-9 and results in apoptosis of the cells. In some embodiments, the iCaspase-9 contains a binding domain that comprises a chemical inducer of dimerization (CID) that mediates dimerization in the presence of the CID, which results in inducible and selective depletion of the chimeric receptor-expressing cells.

Alternatively, in some embodiments a chimeric receptor of the present disclosure may be regulated by utilizing a small molecule or an antibody that deactivates or otherwise inhibits chimeric receptor activity. For example, an antibody may delete the chimeric receptor-expressing cells by inducing antibody dependent cell-mediated cytotoxicity (ADCC). In some embodiments, a chimeric receptor-expressing cell of the present disclosure may further express an antigen that is recognized by a molecule that is capable of inducing cell death by ADCC or complement-induced cell death. For example, a chimeric receptor-expressing cell of the present disclosure may further express a receptor capable of being targeted by an antibody or antibody fragment. Examples of suitable receptors that may be targeted by an antibody or antibody fragment include, without limitation, EpCAM, VEGFR, integrins (e.g., αvβ3, α4, αI^{3/4}, α4β7, α5β1, αvβ3, αv), members of the TNF receptor superfamily (e.g., TRAIL-R1 and TRAIL-R2), PDGF receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof.

In some embodiments, a chimeric receptor-expressing cell of the present disclosure may also express a truncated epidermal growth factor receptor (EGFR) that lacks signaling capacity but retains an epitope that is recognized by molecules capable of inducing ADCC (e.g., WO2011/056894).

In some embodiments, a chimeric receptor-expressing cell of the present disclosure further includes a highly expressing compact marker/suicide gene that combines target epitopes from both CD32 and CD20 antigens in the chimeric receptor-expressing cell, which binds an anti-CD20 antibody (e.g., rituximab) resulting in selective depletion of the chimeric receptor-expressing cell by ADCC. Other methods for depleting chimeric receptor-expressing cells of the present disclosure my include, without limitation, administration of a monoclonal anti-CD52 antibody that selectively binds and targets the chimeric receptor-expressing cell for destruction by inducing ADCC. In some embodiments, the chimeric receptor-expressing cell can be selectively targeted using a chimeric receptor ligand, such as an anti-idiotypic antibody. In some embodiments, the anti-idiotypic antibody can cause effector cell activity, such as ADCC or ADC activity. In some embodiments, the chimeric receptor ligand can be further coupled to an agent that induces cell killing, such as a toxin. In some embodiments, a chimeric receptor-expressing cell of the present disclosure may further express a target protein recognized by a cell depleting agent of the present disclosure. In some embodiments, the target protein is CD20 and the cell depleting agent is an anti-CD20 antibody. In such embodiments, the cell depleting agent is administered once it is desirable to reduce or eliminate the chimeric receptor-expressing cell. In some embodiments, the cell depleting agent is an anti-CD52 antibody.

In some embodiments, a regulated chimeric receptor comprises a set of polypeptides, in which the components of a chimeric receptor of the present disclosure are partitioned on separate polypeptides or members. For example, the set of polypeptides may include a dimerization switch that, when in the presence of a dimerization molecule, can couple the polypeptides to one another to form a functional chimeric receptor.

### Chimeric Receptor-Encoding Polynucleotide Constructs

Certain aspects of the present disclosure relate to polynucleotides (e.g., isolated polynucleotides) encoding one or more chimeric receptors of the present disclosure. In some embodiments, the polynucleotide is an RNA construct, such as a messenger RNA (mRNA) transcript or a modified RNA. In some embodiments, the polynucleotide is a DNA construct.

In some embodiments, a polynucleotide of the present disclosure encodes a chimeric receptor that comprises one or more antigen-binding domain, where each domain binds to a target antigen, a transmembrane domain, and one or more intracellular signaling domains. In some embodiments, the polynucleotide encodes a chimeric receptor that comprises an antigen-binding domain, a transmembrane domain, a primary signaling domain (e.g., CD3-zeta domain), and one or more costimulatory signaling domains. In some embodiments, the polynucleotide further comprises a nucleic acid sequence encoding a spacer region. In some embodiments, the antigen-binding domain is connected to the transmembrane domain by the spacer region. In some embodiments, the nucleic acid further comprises a nucleotide sequence encoding a leader sequence.

The polynucleotides of the present disclosure may be obtained using any suitable recombinant methods known in the art, including, without limitation, by screening libraries from cells expressing the gene of interest, by deriving the gene of interest from a vector known to include the gene, or by isolating the gene of interest directly from cells and tissues containing the gene using standard techniques. Alternatively, the gene of interest may be produced engineeredally.

In some embodiments, a polynucleotide of the present disclosure in comprised within a vector. In some embodiments, a polynucleotide of the present disclosure is expressed in a cell via transposons, a CRISPR/Cas9 system, a TALEN, or a zinc finger nuclease.

In some embodiments, expression of a polynucleotide encoding a chimeric receptor of the present disclosure may be achieved by operably linking the nucleic acid to a promoter and incorporating the construct into an expression vector. A suitable vector can replicate and integrate in eukaryotic cells. Typical cloning vectors contain transcription and translation terminators, initiation sequences, and promoters useful for regulating expression of the desired nucleic acid.

In some embodiments, expression constructs of the present disclosure may also be used for nucleic acid immunization and gene therapy, using standard gene delivery protocols (e.g., U.S. Pat. Nos. 5,399,346, 5,580,859, and 5,589,466). In some embodiments, a vector of the present disclosure is a gene therapy vector.

A polynucleotide of the present disclosure can be cloned into a number of types of vectors. For example, the polynucleotide can be cloned into a vector including, without limitation, a plasmid, a phagemid, a phage derivative, an animal virus, or a cosmid. In some embodiments, the vector may be an expression vector, a replication vector, a probe generation vector, or a sequencing vector.

In some embodiments, the plasmid vector comprises a transposon/transposase system to incorporate the polynucleotides of the present disclosure into the host cell genome. Methods of expressing proteins in immune cells using a transposon and transposase plasmid system are generally described in Chicaybam L, Hum Gene Ther. 2019 April; 30(4):511-522. doi: 10.1089/hum.2018.218; and Ptackovi P, Cytotherapy. 2018 April; 20(4):507-520. doi: 10.1016/j.jcyt.2017.10.001, each of which is hereby incorporated by reference in their entirety. In some embodiments, the transposon system is the Sleeping Beauty transposon/transposase or the piggyBac transposon/transposase.

In some embodiments, an expression vector of the present disclosure may be provided to a cell in the form of a viral vector. Suitable viral vector systems are well known in the art. For example, viral vectors may be derived from retroviruses, adenoviruses, adeno-associated viruses, herpes viruses, and lentiviruses. In some embodiments, a vector of the present disclosure is a lentiviral vector. Lentiviral vectors are suitable for long-term gene transfer as such vectors allow long-term, stable integration of a transgene and its propagation in daughter cells. Lentiviral vectors are also advantageous over vectors derived from onco-retroviruses (e.g., murine leukemia viruses) in that lentiviral vectors can transduce non-proliferating cells. In some embodiments, a vector of the present disclosure is an adenoviral vector (A5/35). In some embodiments, a vector of the present disclosure contains an origin of replication functional in at least one organism, a promoter sequence, convenient restriction endonuclease sites, and one or more selectable markers (e.g., WO01/96584; WO01/29058; and U.S. Pat. No. 6,326,193). A number of viral based systems have been developed for gene transfer into mammalian cells. A selected gene can be inserted into a vector and packaged in retroviral particles using techniques known in the art. The recombinant virus can then be isolated and delivered to mammalian cells either in vivo or ex vivo. A number of retroviral systems are known in the art.

In some embodiments, vectors of the present disclosure include additional promoter elements, such as enhancers that regulate the frequency of transcriptional initiation. Enhancers are typically located in a region that is 30 bp to 110 bp upstream of the start site, although a number of promoters have been shown to contain functional elements downstream of the start site as well. The spacing between promoter elements may be flexible, so that promoter function is preserved when elements are inverted or moved relative to one another. For example, in the thymidine kinase (tk) promoter the spacing between promoter elements can be increased to 50 bp apart before activity begins to decline. Depending on the promoter, individual elements may function either cooperatively or independently to activate transcription. Exemplary promoters may include, without limitation, the SFFV gene promoter, the EFS gene promoter, the CMV IE gene promoter, the EF1a promoter, the ubiquitin C promoter, and the phosphoglycerokinase (PGK) promoter.

In some embodiments, a promoter that is capable of expressing a polynucleotide of the present disclosure in a mammalian cell, such as an immunoresponsive cell of the present disclosure, is the EF1a promoter. The native EF1a promoter drives expression of the alpha subunit of the elongation factor-1 complex, which is responsible for the enzymatic delivery of aminoacyl tRNAs to the ribosome. The EF1a promoter has been widely used in mammalian expression plasmids and has been shown to be effective in driving chimeric receptor expression from polynucleotide cloned into a lentiviral vector.

In some embodiments, a promoter that is capable of expressing a polynucleotide of the present disclosure in a mammalian cell, such as an immunoresponsive cell of the present disclosure, is a constitutive promoter. For example, a suitable constitutive promoter is the spleen focus forming virus (SFFV) promoter. Another example of a suitable constitutive promoter is the immediate early cytomegalovirus (CMV) promoter. The CMV promoter is a strong constitutive promoter that is capable of driving high levels of expression of any polynucleotide sequence operatively linked to the promoter. Other suitable constitutive promoters include, without limitation, a ubiquitin C (UbiC) promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, an actin promoter, a myosin promoter, an elongation factor-1a promoter, a hemoglobin promoter, and a creatine kinase promoter.

In some embodiments, a promoter that is capable of expressing a polynucleotide of the present disclosure in a mammalian cell, such as an immunoresponsive cell of the present disclosure, is an inducible promoter. Use of an inducible promoter may provide a molecular switch that is capable of inducing or repressing expression of a polynucleotide of the present disclosure when the promoter is operatively linked to the polynucleotide. Examples of inducible promoters include, without limitation, a metallothionine promoter, a glucocorticoid promoter, a progesterone promoter, and a tetracycline promoter.

In some embodiments, a vector of the present disclosure may further comprise a signal sequence to facilitate secretion, a polyadenylation signal and transcription terminator, an element allowing episomal replication, and/or elements allowing for selection.

In some embodiments, a vector of the present disclosure can further comprise a selectable marker gene and/or reporter gene to facilitate identification and selection of chimeric receptor-expressing cells from a population of cells that have been transduced with the vector. In some embodiments, the selectable marker may be encoded by a polynucleotide that is separate from the vector and used in a co-transfection procedure. Either selectable marker or reporter gene may be flanked with appropriate regulator sequences to allow expression in host cells. Examples of selectable markers include, without limitation, antibiotic-resistance genes, such as neo and the like.

In some embodiments, reporter genes may be used for identifying transduced cells and for evaluating the functionality of regulatory sequences. As disclosed herein, a reporter gene is a gene that is not present in or expressed by the recipient organism or tissue and that encodes a polypeptide whose expression results in an easily detectable property, such as enzymatic activity. Expression of the reporter gene can be assayed at a suitable time after the polynucleotide has been introduced into the recipient cells. Examples of reporter genes include, without limitation, genes encoding for luciferase, genes encoding for beta-galactosidase, genes encoding for chloramphenicol acetyl transferase, genes encoding for secreted alkaline phosphatase, and genes encoding for green fluorescent protein. Suitable expression systems are well known in the art and may be prepared using known techniques or obtained commercially. In some embodiments, a construct with a minimal 5′ flanking region showing the highest level of expression of the reporter gene is identified as the promoter. Such promoter regions may be linked to a reporter gene and used to evaluate agents for the ability to modulate promoter-driven transcription.

In some embodiments, a vector comprising a polynucleotide sequence encoding a chimeric receptor of the present disclosure further comprises a second polynucleotide encoding a polypeptide that increases the activity of the chimeric receptor.

In embodiments where a chimeric receptor-expressing cell comprises two or more chimeric receptors, a single polynucleotide may encode the two or more chimeric receptors under a single regulatory control element (e.g., promoter) or under separate regulatory control elements for each chimeric receptor-encoding nucleotide sequence comprised in the polynucleotide. In some embodiments where a chimeric receptor-expressing cell comprises two or more chimeric receptors, each chimeric receptor may be encoded by a separate polynucleotide. In some embodiments, each separate polynucleotide comprises its own control element (e.g., promoter). In some embodiments, a single polynucleotide encodes the two or more chimeric receptors and the chimeric receptor-encoding nucleotide sequences are in the same reading frame and are expressed as a single polypeptide chain. In such embodiments, the two or more chimeric receptors may be separated by one or more peptide cleavage sites, such as auto-cleavage sites or substrates for an intracellular protease. Suitable peptide cleavage sites may include, without limitation, a T2A peptide cleavage site, a P2A peptide cleavage site, an E2A peptide cleavage sire, and an F2A peptide cleavage site. In some embodiments, the two or more chimeric receptors comprise a T2A peptide cleavage site. In some embodiments, the two or more chimeric receptors comprise an E2A peptide cleavage site. In some embodiments, the two or more chimeric receptors comprise a T2A and an E2A peptide cleavage site.

Methods of introducing and expressing genes into a cell are well known in the art. For example, in some embodiments, an expression vector can be transferred into a host cell by physical, chemical, or biological means. Examples of physical means for introducing a polynucleotide into a host cell include, without limitation, calcium phosphate precipitation, lipofection, particle bombardment, microinjection, and electroporation. Examples of chemical means for introducing a polynucleotide into a host cell include, without limitation, colloidal dispersion systems, macromolecule complexes, nanocapsules, microspheres, beads, and lipid-based systems including oil-in-water emulsions, micelles, mixed micelles, and liposomes. Examples of biological means for introducing a polynucleotide into a host cell include, without limitation, the use of DNA and RNA vectors.

In some embodiments, liposomes may be used as a non-viral delivery system to introduce a polynucleotide or vector of the present disclosure into a host cell in vitro, ex vivo, or in vivo. In some embodiments, the polynucleotide may be associated with a lipid, for example by being encapsulated in the aqueous interior of a liposome, being interspersed within the lipid bilayer of a liposome, being attached to a liposome via a linking molecule that is associated with both the liposome and the polynucleotide, being entrapped in a liposome, being complexed with a liposome, being dispersed in a solution containing a lipid, being mixed with a lipid, being combined with a lipid, being contained as a suspension in a lipid, being contained or complexed with a micelle, or otherwise being associated with a lipid. As disclosed herein, lipid-associated polynucleotide or vector compositions are not limited to any particular structure in solution. In some embodiments, such compositions may be present in a bilayer structure, as micelles or with a “collapsed” structure. Such compositions may also be interspersed in a solution, forming aggregates that are not uniform in size or shape. As disclosed herein, lipids are fatty substances that may be naturally occurring or engineered. In some embodiments, lipids can include the fatty droplets that naturally occur in the cytoplasm or the class of compounds that contain long-chain aliphatic hydrocarbons and their derivatives, such as fatty acids, alcohols, amines, amino alcohols, and aldehydes. Suitable lipids may be obtained from commercial sources and include, without limitation, dimyristyl phosphatidylcholine (“DMPC”), dicetylphosphate (“DCP”), cholesterol, and dimyristylphosphatidylglycerol (“DMPG”). Stock solutions of lipids in chloroform or chloroform/methanol can be stored at about −20° C. Chloroform is used as the solvent, as it is more readily evaporated than methanol. As used herein, a “liposome” may encompass a variety of single and multilamellar lipid vehicles formed by the generation of enclosed lipid bilayers or aggregates. In some embodiments, liposomes can be characterized as having vesicular structures with a phospholipid bilayer membrane and an inner aqueous medium. In some embodiments, multilamellar liposomes may have multiple lipid layers separated by aqueous medium. Multilamellar liposomes can form spontaneously when phospholipids are suspended in an excess of aqueous solution. In some embodiments, lipid components may undergo self-rearrangement before the formation of closed structures and can entrap water and dissolved solutes between the lipid bilayers. In some embodiments, the lipids may assume a micellar structure or merely exist as nonuniform aggregates of lipid molecules.

In some embodiments, a polynucleotide or vector of the present disclosure is introduced into a mammalian host cell, such as an immunoresponsive cell of the present disclosure. In some embodiments, the presence of a polynucleotide or vector of the present disclosure in a host cell may be confirmed by any suitable assay known in the art, including without limitation Southern blot assays, Northern blot assays, RT-PCR, PCR, ELISA assays, and Western blot assays.

In some embodiments, a polynucleotide or vector of the present disclosure is stably transduced into an immunoresponsive cell of the present disclosure. In some embodiments, cells that exhibit stable expression of the polynucleotide or vector express the encoded chimeric receptor for at least 1 week, at least 2 weeks, at least 3 weeks, at least 4 weeks, at least 5 weeks, at least 6 weeks, at least 7 weeks, at least 8 weeks, at least 3 months, at least 6 months, at least 9 months, or at least 12 months after transduction.

In embodiments where a chimeric receptor of the present disclosure is transiently expressed in a cell, a chimeric receptor-encoding polynucleotide or vector of the present disclosure is transfected into an immunoresponsive cell of the present disclosure. In some embodiments the immunoresponsive cell expresses the chimeric receptor for about 4 days, about 5 days, about 6 days, about 7 days, about 8 days, about 9 days, about 10 days, about 11 days, about 12 days, about 13 days, about 14 days, or about 15 days after transfection.

### Methods of Engineering Cells

Also provided herein are compositions and methods for engineering cells to produce one or more effectors molecules encoded by any engineered nucleic acid comprising the first and second expression cassettes as described herein or otherwise known in the art.

In general, cells are engineered to produce effector molecules through introduction (i.e., delivery) of one or more polynucleotides of the present disclosure comprising the first promoter and the exogenous polynucleotide sequence encoding at least one effector molecule and/or the second expression cassette comprising an second exogenous sequence encoding one or more effector molecules into the cell's cytosol and/or nucleus. For example, the polynucleotide expression cassettes encoding the one or more effector molecules can be any of the engineered nucleic acids described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

In some embodiments, the engineered cell is transduced using an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. In some embodiments, the oncolytic virus is a recombinant oncolytic virus comprising the first expression cassette and the second expression cassette. In some embodiments, the oncolytic virus further comprises the third expression cassette.

The virus, including any of the oncolytic viruses described herein, can be a recombinant virus that encodes one more transgenes encoding one or more effector molecules, such as any of the engineered nucleic acids described herein. The virus, including any of the oncolytic viruses described herein, can be a recombinant virus that encodes one more transgenes encoding one or more of the two or more effector molecules, such as any of the engineered nucleic acids described herein. In some embodiments, the cell is engineered via transduction with an oncolytic virus.

### Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, a engineered nucleic acid can also encompass a engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, a engineered virally-derived nucleic acid, e.g., a recombinant virus or a engineered virus, can encode one or more transgenes, including, but not limited to, any of the engineered nucleic acids described herein that encode one or more effector molecules. The one or more transgenes encoding the one or more effector molecules can be configured to express the one or more effector molecules. A viral vector-based delivery platform can encode one or more genes in addition to the one or more transgenes (e.g., transgenes encoding the one or more effector molecules), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more effector molecules. One viral vector can deliver more than one engineered nucleic acids, such as one vector that delivers engineered nucleic acids that are configured to produce two or more effector molecules. More than one viral vector can deliver more than one engineered nucleic acids, such as more than one vector that delivers one or more engineered nucleic acid configured to produce one or more effector molecules. The number of viral vectors used can depend on the packaging capacity of the above-mentioned viral vector-based vaccine platforms, and one skilled in the art can select the appropriate number of viral vectors.

In general, any of the viral vector-based systems can be used for the in vitro production of molecules, such as effector molecules, or used in vivo and ex vivo gene therapy procedures, e.g., for in vivo delivery of the engineered nucleic acids encoding one or more effector molecules. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., a engineered cell) can express, and in some case secrete, the one or more effector molecules. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., a engineered nucleic acid) encoding one or more effector molecules. The transgenes encoding the one or more effector molecules can be configured to express the one or more effector molecules.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more effector molecules) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of effector molecules, or used in vivo and ex vivo gene therapy procedures, e.g., for in vivo delivery of the engineered nucleic acids encoding one or more effector molecules (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

### Lipid Structure Delivery Systems

Engineered nucleic acids of the present disclosure (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., a engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, a engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are engineered lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be engineered or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical engineered lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of a engineered nucleic acid and/or viral delivery system can be used to avoid degradation, while also avoiding potential off-target affects. In certain examples, a engineered nucleic acid and/or viral delivery system is fully encapsulated within the delivery vehicle, such as within the aqueous interior of an LNP. Encapsulation of a engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with a engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., a engineered nucleic acid and/or viral delivery system) can be further treated or engineered to prepare them for administration.

### Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery-A Review. Nanomaterials 2017, 7(5), 94), herein incorporated by reference for all purposes.

### Genomic Editing Systems

A genomic editing systems can be used to engineer a host genome to encode a engineered nucleic acid, such as a engineered nucleic acid of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate a engineered nucleic acid, such as a engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., a engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode a engineered nucleic acid, such as a engineered nucleic acid of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more effector molecules).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode a engineered nucleic acid, such as a engineered nucleic acid encoding one or more of the effector molecules described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and a RNA(s) that directs cleavage to a particular target sequence. An exemplary CRISPR-mediated gene editing system is the CRISPR/Cas9 systems comprised of a Cas9 nuclease and a RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is a engineered site-specific nuclease, which is composed of the DNA-binding domain of TALE (transcription activator-like effectors) and the catalytic domain of restriction endonuclease Fok1. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other Engineering Delivery Systems

Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60.), each herein incorporated by reference for all purposes.

### Methods of Use

Methods for treatment of diseases are also encompassed by this disclosure. Said methods include administering a therapeutically effective amount of a engineered nucleic acid, engineered cell, or isolated cell as described above. In some aspects, provided herein are methods of treating a subject in need thereof, the method comprising administering a therapeutically effective dose of any of the engineered cells, isolated cells, or compositions disclosed herein.

In some aspects, provided herein is a method of increasing expression of a target gene, e.g. a tumor suppressor gene.

In some aspects, provided herein a methods of increasing expression of a target gene, e.g. an immunomodulatory gene. Exemplary immunomodulatory genes include, for example and without limitation, cytokines, chemokines, receptors thereof, and derivatives thereof.

In some aspects, provided herein are methods of stimulating a cell-mediated immune response to a tumor cell in a subject, the method comprising administering to a subject having a tumor a therapeutically effective dose of any of the engineered cells, isolated cells, or compositions disclosed herein.

In some aspects, provided herein are methods of providing an anti-tumor immunity in a subject, the method comprising administering to a subject in need thereof a therapeutically effective dose of any of the engineered cells, isolated cells, or compositions disclosed herein.

In some aspects, provided herein are methods of treating a subject having cancer, the method comprising administering a therapeutically effective dose of any of the engineered cells, isolated cells, or compositions disclosed herein.

In some aspects, provided herein are methods of reducing tumor volume in a subject, the method comprising administering to a subject having a tumor a composition comprising any of the engineered cells, isolated cells, or compositions disclosed herein.

In some embodiments, the administering comprises systemic administration. In some embodiments, the administering comprises intratumoral administration. In some embodiments, the isolated cell is derived from the subject. In some embodiments, the isolated cell is allogeneic with reference to the subject.

In some embodiments, the method further comprises administering a checkpoint inhibitor. the checkpoint inhibitor is selected from: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GAL9 antibody, an anti-A2AR antibody, an anti-phosphatidylserine antibody, an anti-CD27 antibody, an anti-TNFa antibody, an anti-TREM1 antibody, and an anti-TREM2 antibody. In some embodiments, the method further comprises administering an anti-CD40 antibody.

In some embodiments, the tumor is selected from: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

Some methods comprise selecting a subject (or patient population) having a tumor (or cancer) and treating that subject with engineered cells or delivery vehicles that modulate tumor-mediated immunosuppressive mechanisms.

The methods provided herein also include delivering a preparation of engineered cells or delivery vehicles. A preparation, in some embodiments, is a substantially pure preparation, containing, for example, less than 5% (e.g., less than 4%, 3%, 2%, or 1%) of cells other tha engineered cells. A preparation may comprise 1×10⁵ cells/kg to 1×10⁷ cells/kg cells.

### In Vivo Expression

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the effector molecules described herein. The methods provided herein also include delivering a composition in vivo capable of producing two or more of the effector molecules described herein. Compositions capable of in vivo production of effector molecules include, but are not limited to, any of the engineered nucleic acids described herein. Compositions capable of in vivo production of effector molecules can be a naked mRNA or a naked plasmid.

### Pharmaceutical Compositions

The engineered nucleic acid or engineered cell can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or engineered oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Whether it is a polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Kits

Certain aspects of the present disclosure relate to kits for the treatment and/or prevention of disease or disorder. In certain embodiments, the disease or disorder is a cancer (e.g., solid tumors). In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., immunoresponsive cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing cancer (e.g., a solid tumor). In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### Enumerated Embodiments

Embodiment 1: An engineered macrophage-specific promoter system comprising: a regulatory element and a heterologous payload;
wherein the regulatory element exhibits greater activity in an M1 macrophage compared to an M2 or M0 macrophage, and wherein the regulatory element is or comprises an enhancer region that is derived from a promoter of a gene that is more highly expressed in M1 macrophage compared to M2 or M0 macrophages.
   Embodiment 2: The engineered macrophage-specific promoter system of embodiment 1, wherein the regulatory element is at least 100, 200, 300, 400, 500, 600, 700, 800, 900, 1000, 1100, 1200, 1300, 1400, 1500, 1600, 1700, 1800, 1900, 2000, 2200, 2400, 2500, 2600, 2800, or 3000 base pairs in length.
   Embodiment 3: The engineered macrophage-specific promoter system of embodiment 1 or 2, wherein the regulatory element is derived from a promoter of a gene, wherein the gene is selected from the group consisting of CCL19, CCR7, CXCL11, GBP5, IDO1, UBD, and UNQ6494.1.
   Embodiment 4: The engineered macrophage-specific promoter system of any one of embodiments 1-3, wherein the regulatory element is derived from a CCL19 promoter.
   Embodiment 5: The engineered macrophage-specific promoter system of any one of embodiments 1-3, wherein the regulatory element comprises the nucleotide sequence of SEQ ID NO: 132.
   Embodiment 6: The engineered macrophage-specific promoter system of any one of embodiments 1-3, wherein the regulatory element is derived from a CCR7 promoter.
   Embodiment 7: The engineered macrophage-specific promoter system of any one of embodiments 1-3, wherein the regulatory element comprises the nucleotide sequence of SEQ ID NO: 133.
   Embodiment 8: The engineered macrophage-specific promoter system of any one of embodiments 1-3, wherein the regulatory element is derived from a CXCL11 promoter.
   Embodiment 9: The engineered macrophage-specific promoter system of any one of embodiments 1-3, wherein the regulatory element comprises the nucleotide sequence of SEQ ID NO: 134.
   Embodiment 10: The engineered macrophage-specific promoter system of any one of embodiments 1-3, wherein the regulatory element is derived from a GBP5 promoter.
   Embodiment 11: The engineered macrophage-specific promoter system of any one of embodiments 1-3, wherein the regulatory element comprises the nucleotide sequence of SEQ ID NO: 135.
   Embodiment 12: The engineered macrophage-specific promoter system of any one of embodiments 1-3, wherein the regulatory element is derived from an IDO1 promoter.
   Embodiment 13: The engineered macrophage-specific promoter system of any one of embodiments 1-3, wherein the regulatory element comprises the nucleotide sequence of SEQ ID NO: 136.
   Embodiment 14: The engineered macrophage-specific promoter system of any one of embodiments 1-3, wherein the regulatory element is derived from a UBD promoter.
   Embodiment 15: The engineered macrophage-specific promoter system of any one of embodiments 1-3, wherein the regulatory element comprises the nucleotide sequence of SEQ ID NO: 137.
   Embodiment 16: The engineered macrophage-specific promoter system of any one of embodiments 1-3, wherein the regulatory element is derived from a UNQ6494.1 promoter.
   Embodiment 17: The engineered macrophage-specific promoter system of any one of embodiments 1-3, wherein the regulatory element comprises the nucleotide sequence of SEQ ID NO: 138.
   Embodiment 18: The engineered macrophage-specific promoter system of any one of embodiments 1-17, wherein the heterologous payload is selected from the group consisting of: transcriptions factors, cytokines, receptors, enzymes, chemokines, antibodies, fragments of antibodies, miRNAs, and shRNAs.
   Embodiment 19: An engineered macrophage-specific promoter system comprising
i. a regulatory element; and
ii. a heterologous payload;
wherein the regulatory element exhibits greater activity in an M2 macrophage compared to an M1 or M0 macrophage, and wherein the regulatory element is or comprises an enhancer region that is derived from a promoter of a gene that is more highly expressed in M2 macrophage compared to M1 or M0 macrophages.
   Embodiment 20: The engineered macrophage-specific promoter system of embodiment 19, wherein the regulatory element is at least 100, 200, 300, 400, 500, 600, 700, 800, 900, 1000, 1100, 1200, 1300, 1400, 1500, 1600, 1700, 1800, 1900, 2000, 2200, 2400, 2500, 2600, 2800, or 3000 base pairs in length.
   Embodiment 21: The engineered macrophage-specific promoter system of embodiment 19 or 20, wherein the regulatory element is a promoter of a gene, wherein the gene is selected from the group consisting of CD28, SOCS3, PLXDC1, IL7R and ZNF704.
   Embodiment 22: The engineered macrophage-specific promoter system of any one of embodiments 19-21, wherein the regulatory element is derived from a CD28 promoter.
   Embodiment 23: The engineered macrophage-specific promoter system of any one of embodiments 19-21, wherein the regulatory element comprises the nucleotide sequence of SEQ ID NO: 139.
   Embodiment 24: The engineered macrophage-specific promoter system of any one of embodiments 19-21, wherein the regulatory element is derived from a PLXDC1 promoter
   Embodiment 25: The engineered macrophage-specific promoter system of any one of embodiments 19-21, wherein the regulatory element comprises the nucleotide sequence of SEQ ID NO: 140.
   Embodiment 26: The engineered macrophage-specific promoter system of any one of embodiments 19-21, wherein the regulatory element is derived from a ZNF704 promoter.
   Embodiment 27: The engineered macrophage-specific promoter system of any one of embodiments 19-21, wherein the regulatory element comprises the nucleotide sequence of SEQ ID NO: 141.
   Embodiment 28: The engineered macrophage-specific promoter system of any one of embodiments 19-21, wherein the regulatory element is derived from a IL7R promoter.
   Embodiment 29: The engineered macrophage-specific promoter system of any one of embodiments 19-21, wherein the regulatory element comprises the nucleotide sequence of SEQ ID NO: 392.
   Embodiment 30: The engineered macrophage-specific promoter system of any one of embodiments 19-21, wherein the regulatory element is derived from a SOCS3 promoter.
   Embodiment 31: The engineered macrophage-specific promoter system of any one of embodiments 19-21, wherein the regulatory element comprises the nucleotide sequence of SEQ ID NO: 393.
   Embodiment 32: The engineered macrophage-specific promoter system of embodiment 19 or 20, wherein the regulatory element is a promoter of a gene, wherein the gene is selected from the group consisting of: LNCAROD, MRC1, and ID3.
   Embodiment 33: The engineered macrophage-specific promoter system of embodiment 32, wherein the regulatory element is derived from a LNCAROD promoter.
   Embodiment 34: The engineered macrophage-specific promoter system of embodiment 33, wherein the regulatory element derived from the LNCAROD promoter comprises:
(i) a nucleotide sequence having at least 75%, at least 80%, at least 85%, at least 90%, at least 95%, at least 96%, at least 97%, at least 98%, at least 99% or 100% sequence identity to SEQ ID NO: 414,
(ii) a nucleotide sequence having at least 75%, at least 80%, at least 85%, at least 90%, at least 95%, at least 96%, at least 97%, at least 98%, at least 99% or 100% sequence identity to SEQ ID NO: 415,
(iii) a nucleotide sequence having at least 75%, at least 80%, at least 85%, at least 90%, at least 95%, at least 96%, at least 97%, at least 98%, at least 99% or 100% sequence identity to SEQ ID NO: 416, or
(iv) a nucleotide sequence having at least 75%, at least 80%, at least 85%, at least 90%, at least 95%, at least 96%, at least 97%, at least 98%, at least 99% or 100% sequence identity to SEQ ID NO: 417.
   Embodiment 35: The engineered macrophage-specific promoter system of embodiment 32, wherein the regulatory element is derived from an ID3 promoter.
   Embodiment 36: The engineered macrophage-specific promoter system of embodiment 35, wherein the regulatory element derived from the ID3 promoter comprises a nucleotide sequence having at least 75%, at least 80%, at least 85%, at least 90%, at least 95%, at least 96%, at least 97%, at least 98%, at least 99% or 100% sequence identity to SEQ ID NO: 418.
   Embodiment 37: The engineered macrophage-specific promoter system of embodiment 32, wherein the regulatory element is derived from an MRC1 promoter.
   Embodiment 38: The engineered macrophage-specific promoter system of embodiment 37, wherein the regulatory element derived from the MRC1 promoter comprises a nucleotide sequence having at least 75%, at least 80%, at least 85%, at least 90%, at least 95%, at least 96%, at least 97%, at least 98%, at least 99% or 100% sequence identity to SEQ ID NO: 419.
   Embodiment 39: The engineered macrophage-specific promoter system of any one of embodiments 19-38, wherein the heterologous payload is selected from the group consisting of transcriptions factors, cytokines, receptors, enzymes, chemokines, antibodies, fragments of antibodies, miRNAs, and shRNAs.
   Embodiment 40: An engineered macrophage-specific promoter comprising an ablation of at least one nucleotide motif, wherein the ablation increases specific activity of the engineered macrophage-specific promoter in M1 macrophages, as compared to activity of a corresponding macrophage-specific promoter lacking the ablation in M1 macrophages.
   Embodiment 41: The engineered macrophage-specific promoter of embodiment 40, wherein the corresponding macrophage-specific promoter lacking the ablation in M1 macrophages is a wildtype macrophage promoter, and wherein the wildtype macrophage promoter comprises a sequence selected from the group consisting of SEQ ID NOs: 132-138.
   Embodiment 42: The engineered macrophage-specific promoter of embodiment 40 or 41, wherein the wildtype macrophage promoter comprises the nucleotide sequence of SEQ ID NO: 132.
   Embodiment 43: The engineered macrophage-specific promoter of any one of embodiments 40-42, wherein the at least one nucleotide motif comprises a motif having a sequence within the nucleotide sequence of SEQ ID NO: 132, wherein the motif comprises a sequence selected from the group consisting of: position 63 to position 73 of SEQ ID NO: 132, position 80 to position 102 of SEQ ID NO: 132, position 141 to position 162 of SEQ ID NO: 132, position 212 to position 222 of SEQ ID NO: 132, position 229 to position 251 of SEQ ID NO: 132, position 307 to position 361 of SEQ ID NO: 132, position 365 to position 376 of SEQ ID NO: 132, position 559 to position 571 of SEQ ID NO: 132, position 617 to position 633 of SEQ ID NO: 132, position 782 to position 799 of SEQ ID NO: 132, position 852 to position 871 of SEQ ID NO: 132, position 886 to position 920 of SEQ ID NO: 132, position 933 to position 959 of SEQ ID NO: 132, position 1002 to position 1028 of SEQ ID NO: 132, position 1032 to position 1045 of SEQ ID NO: 132, position 1064 to position 1087 of SEQ ID NO: 132, position 1169 to position 1192 of SEQ ID NO: 132, position 1212 to position 1232 of SEQ ID NO: 132, position 1257 to position 1275 of SEQ ID NO: 132, position 1310 to position 1333 of SEQ ID NO: 132, position 1381 to position 1434 of SEQ ID NO: 132, position 1698 to position 1753 of SEQ ID NO: 132, position 1783 to position 1826 of SEQ ID NO: 132, position 1909 to position 1927 of SEQ ID NO: 132, position 1946 to position 1961 of SEQ ID NO: 132.
   Embodiment 44: The engineered macrophage-specific promoter of any one of embodiments 40-43, wherein the ablation comprises a substitution or deletion of one or more nucleotides of the at least one nucleotide motif.
   Embodiment 45: The engineered macrophage-specific promoter of any one of embodiments 40-44, wherein the at least one nucleotide motif comprises a motif having a sequence within the nucleotide sequence of SEQ ID NO: 132, wherein the motif corresponds to position 63 to position 73 of SEQ ID NO: 132.
   Embodiment 46: The engineered macrophage-specific promoter of any one of embodiments 40-44, wherein the ablation comprises a nucleotide substitution comprising the sequence CTTACCTACT (SEQ ID NO: 171) from position 63 to position 73 of SEQ ID NO: 132.
   Embodiment 47: The engineered macrophage-specific promoter of any one of embodiments 40-44, wherein the ablation comprises nucleotide deletions of position 63 to position 73 of SEQ ID NO: 132.
   Embodiment 48: The engineered macrophage-specific promoter of any one of embodiments 40-47, wherein the at least one nucleotide motif comprises a motif having a sequence within the nucleotide sequence of SEQ ID NO: 132, wherein the motif corresponds to position 80 to position 102 of SEQ ID NO: 132.
   Embodiment 49: The engineered macrophage-specific promoter of any one of embodiments 40-47, wherein the ablation comprises a nucleotide substitution comprising the sequence AATTCAGACGACAAACCATTCT (SEQ ID NO: 173) from position 80 to position 102 of SEQ ID NO: 132.
   Embodiment 50: The engineered macrophage-specific promoter of any one of embodiments 40-47, wherein the ablation comprises nucleotide deletions of position 80 to position 102 of SEQ ID NO: 132.
   Embodiment 51: The engineered macrophage-specific promoter of any one of embodiments 40-50, wherein the at least one nucleotide motif comprises a motif having a sequence within the nucleotide sequence of SEQ ID NO: 132, wherein the motif corresponds to position 141 to position 162 of SEQ ID NO: 132.
   Embodiment 52: The engineered macrophage-specific promoter of any one of embodiments 40-50, wherein the ablation comprises a nucleotide substitution comprising the sequence TTCTAAGTCCAATTCACGACA (SEQ ID NO:175) from position 141 to position 162 of SEQ ID NO:132.
   Embodiment 53: The engineered macrophage-specific promoter of any one of embodiments 40-50, wherein the ablation comprises nucleotide deletions of position 141 to position 162 of SEQ ID NO: 132.
   Embodiment 54: The engineered macrophage-specific promoter of any one of embodiments 40-53, wherein the at least one nucleotide motif comprises a motif having a sequence within the nucleotide sequence of SEQ ID NO: 132, wherein the motif corresponds to position 212 to position 222 of SEQ ID NO: 132.
   Embodiment 55: The engineered macrophage-specific promoter of any one of embodiments 40-53, wherein the ablation comprises a nucleotide substitution comprising the sequence GTTGAAGCTT (SEQ ID NO:177) from position 212 to position 222 of SEQ ID NO: 132.
   Embodiment 56: The engineered macrophage-specific promoter of any one of embodiments 40-53, wherein the ablation comprises nucleotide deletions of position 212 to position 222 of SEQ ID NO: 132.
   Embodiment 57: The engineered macrophage-specific promoter of any one of embodiments 40-56, wherein the at least one nucleotide motif comprises a motif having a sequence within the nucleotide sequence of SEQ ID NO: 132, wherein the motif corresponds to position 229 to position 251 of SEQ ID NO: 132.
   Embodiment 58: The engineered macrophage-specific promoter of any one of embodiments 40-56, wherein the ablation comprises a nucleotide substitution comprising the sequence GAGTCGTCAGACTCAATTATTA (SEQ ID NO:179) from position 229 to position 251 of SEQ ID NO: 132.
   Embodiment 59: The engineered macrophage-specific promoter of any one of embodiments 40-56, wherein the ablation comprises nucleotide deletions of position 229 to position 251 of SEQ ID NO: 132.
   Embodiment 60: The engineered macrophage-specific promoter of any one of embodiments 40-59, wherein the at least one nucleotide motif comprises a motif having a sequence within the nucleotide sequence of SEQ ID NO: 132, wherein the motif corresponds to position 307 to position 361 of SEQ ID NO: 132.
   Embodiment 61: The engineered macrophage-specific promoter of any one of embodiments 40-59, wherein the ablation comprises a nucleotide substitution comprising the sequence AATTGGAACCACGTATCTACTGCATTGTAACTACAACAGCTCGAGGTATTAGAT (SEQ ID NO:181) from position 307 to position 361 of SEQ ID NO: 132.
   Embodiment 62: The engineered macrophage-specific promoter of any one of embodiments 40-59, wherein the ablation comprises nucleotide deletions of position 307 to position 361 of SEQ ID NO: 132.
   Embodiment 63: The engineered macrophage-specific promoter of any one of embodiments 40-62, wherein the at least one nucleotide motif comprises a motif having a sequence within the nucleotide sequence of SEQ ID NO: 132, wherein the motif corresponds to position 365 to position 376 of SEQ ID NO: 132.
   Embodiment 64: The engineered macrophage-specific promoter of any one of embodiments 40-62, wherein the ablation comprises a nucleotide substitution comprising the sequence GGTGAATTTTC (SEQ ID NO:183) from position 365 to position 376 of SEQ ID NO: 132.
   Embodiment 65: The engineered macrophage-specific promoter of any one of embodiments 40-62, wherein the ablation comprises nucleotide deletions of position 365 to position 376 of SEQ ID NO: 132.
   Embodiment 66: The engineered macrophage-specific promoter of any one of embodiments 40-65, wherein the at least one nucleotide motif comprises a motif having a sequence within the nucleotide sequence of SEQ ID NO: 132, wherein the motif corresponds to position 559 to position 571 of SEQ ID NO: 132.
   Embodiment 67: The engineered macrophage-specific promoter of any one of embodiments 40-65, wherein the ablation comprises a nucleotide substitution comprising the sequence TACTCATCACTA (SEQ ID NO:185) from position 559 to position 571 of SEQ ID NO: 132.
   Embodiment 68: The engineered macrophage-specific promoter of any one of embodiments 40-65, wherein the ablation comprises nucleotide deletions of position 559 to position 571 of SEQ ID NO: 132.
   Embodiment 69: The engineered macrophage-specific promoter of any one of embodiments 40-68, wherein the at least one nucleotide motif comprises a motif having a sequence within the nucleotide sequence of SEQ ID NO: 132, wherein the motif corresponds to position 617 to position 633 of SEQ ID NO: 132.
   Embodiment 70: The engineered macrophage-specific promoter of any one of embodiments 40-68, wherein the ablation comprises a nucleotide substitution comprising the sequence TGCTAGTTGTCCAATA (SEQ ID NO:187) from position 617 to position 633 of SEQ ID NO: 132.
   Embodiment 71: The engineered macrophage-specific promoter of any one of embodiments 40-68, wherein the ablation comprises nucleotide deletions of position 617 to position 633 of SEQ ID NO: 132.
   Embodiment 72: The engineered macrophage-specific promoter of any one of embodiments 40-71, wherein the at least one nucleotide motif comprises a motif having a sequence within the nucleotide sequence of SEQ ID NO: 132, wherein the motif corresponds to position 782 to position 799 of SEQ ID NO: 132.
   Embodiment 73: The engineered macrophage-specific promoter of any one of embodiments 40-71, wherein the ablation comprises a nucleotide substitution comprising the sequence CGTGTGTCATATAGAAT (SEQ ID NO:189) from position 782 to position 799 of SEQ ID NO: 132.
   Embodiment 74: The engineered macrophage-specific promoter of any one of embodiments 40-71, wherein the ablation comprises nucleotide deletions of position 782 to position 799 of SEQ ID NO: 132.
   Embodiment 75: The engineered macrophage-specific promoter of any one of embodiments 40-74, wherein the at least one nucleotide motif comprises a motif having a sequence within the nucleotide sequence of SEQ ID NO: 132, wherein the motif corresponds to position 852 to position 871 of SEQ ID NO: 132.
   Embodiment 76: The engineered macrophage-specific promoter of any one of embodiments 40-74, wherein the ablation comprises a nucleotide substitution comprising the sequence AACAGTCTAAGTCCTCAAA (SEQ ID NO:191) from position 852 to position 871 of SEQ ID NO: 132.
   Embodiment 77: The engineered macrophage-specific promoter of any one of embodiments 40-74, wherein the ablation comprises nucleotide deletions of position 852 to position 871 of SEQ ID NO: 132.
   Embodiment 78: The engineered macrophage-specific promoter of any one of embodiments 40-77, wherein the at least one nucleotide motif comprises a motif having a sequence within the nucleotide sequence of SEQ ID NO: 132, wherein the motif corresponds to position 886 to position 920 of SEQ ID NO: 132.
   Embodiment 79: The engineered macrophage-specific promoter of any one of embodiments 40-77, wherein the ablation comprises a nucleotide substitution comprising the sequence ACTCTACGGAAGTAGCTTGTTTAAAACCTATAGT (SEQ ID NO:193) from position 886 to position 920 of SEQ ID NO: 132.
   Embodiment 80: The engineered macrophage-specific promoter of any one of embodiments 40-77, wherein the ablation comprises nucleotide deletions of position 886 to position 920 of SEQ ID NO: 132.
   Embodiment 81: The engineered macrophage-specific promoter of any one of embodiments 40-80, wherein the at least one nucleotide motif comprises a motif having a sequence within the nucleotide sequence of SEQ ID NO: 132, wherein the motif corresponds to position 933 to position 959 of SEQ ID NO: 132.
   Embodiment 82: The engineered macrophage-specific promoter of any one of embodiments 40-80, wherein the ablation comprises a nucleotide substitution comprising the sequence GTTCTACTAGTACAAAGGTACCAGTA (SEQ ID NO:195) from position 933 to position 959 of SEQ ID NO: 132.
   Embodiment 83: The engineered macrophage-specific promoter of any one of embodiments 40-80, wherein the ablation comprises nucleotide deletions of position 933 to position 959 of SEQ ID NO: 132.
   Embodiment 84: The engineered macrophage-specific promoter of any one of embodiments 40-83, wherein the at least one nucleotide motif comprises a motif having a sequence within the nucleotide sequence of SEQ ID NO: 132, wherein the motif corresponds to position 1002 to position 1028 of SEQ ID NO: 132.
   Embodiment 85: The engineered macrophage-specific promoter of any one of embodiments 40-83, wherein the ablation comprises a nucleotide substitution comprising the sequence TGAGTAAACTAACTTTCAACCGCTCT (SEQ ID NO:197) from position 1002 to position 1028 of SEQ ID NO: 132.
   Embodiment 86: The engineered macrophage-specific promoter of any one of embodiments 40-83, wherein the ablation comprises nucleotide deletions of position 1002 to position 1028 of SEQ ID NO: 132.
   Embodiment 87: The engineered macrophage-specific promoter of any one of embodiments 40-86, wherein the at least one nucleotide motif comprises a motif having a sequence within the nucleotide sequence of SEQ ID NO: 132, wherein the motif corresponds to position 1032 to position 1045 of SEQ ID NO: 132.
   Embodiment 88: The engineered macrophage-specific promoter of any one of embodiments 40-86, wherein the ablation comprises a nucleotide substitution comprising the sequence TCGTTACCATCTT (SEQ ID NO:199) from position 1032 to position 1045 of SEQ ID NO: 132.
   Embodiment 89: The engineered macrophage-specific promoter of any one of embodiments 40-86, wherein the ablation comprises nucleotide deletions of position 1032 to position 1045 of SEQ ID NO: 132.
   Embodiment 90: The engineered macrophage-specific promoter of any one of embodiments 40-89, wherein the at least one nucleotide motif comprises a motif having a sequence within the nucleotide sequence of SEQ ID NO: 132, wherein the motif corresponds to position 1064 to position 1087 of SEQ ID NO: 132.
   Embodiment 91: The engineered macrophage-specific promoter of any one of embodiments 40-89, wherein the ablation comprises a nucleotide substitution comprising the sequence AAACACCGTTTTGCTGTAATATC (SEQ ID NO:201) from position 1064 to position 1087 of SEQ ID NO: 132.
   Embodiment 92: The engineered macrophage-specific promoter of any one of embodiments 40-89, wherein the ablation comprises nucleotide deletions of position 1064 to position 1087 of SEQ ID NO: 132.
   Embodiment 93: The engineered macrophage-specific promoter of any one of embodiments 40-92, wherein the at least one nucleotide motif comprises a motif having a sequence within the nucleotide sequence of SEQ ID NO: 132, wherein the motif corresponds to position 1169 to position 1192 of SEQ ID NO: 132.
   Embodiment 94: The engineered macrophage-specific promoter of any one of embodiments 40-92, wherein the ablation comprises a nucleotide substitution comprising the sequence CGCGTAGAACTTCGTAACATTAA (SEQ ID NO:203) from position 1169 to position 1192 of SEQ ID NO: 132.
   Embodiment 95: The engineered macrophage-specific promoter of any one of embodiments 40-92, wherein the ablation comprises nucleotide deletions of position 1169 to position 1192 of SEQ ID NO: 132.
   Embodiment 96: The engineered macrophage-specific promoter of any one of embodiments 40-95, wherein the at least one nucleotide motif comprises a motif having a sequence within the nucleotide sequence of SEQ ID NO: 132, wherein the motif corresponds to position 1212 to position 1232 of SEQ ID NO: 132.
   Embodiment 97: The engineered macrophage-specific promoter of any one of embodiments 40-95, wherein the ablation comprises a nucleotide substitution comprising the sequence AGATAACGCCGTCATTGTAT (SEQ ID NO:205) from position 1212 to position 1232 of SEQ ID NO: 132.
   Embodiment 98: The engineered macrophage-specific promoter of any one of embodiments 40-95, wherein the ablation comprises nucleotide deletions of position 1212 to position 1232 of SEQ ID NO: 132.
   Embodiment 99: The engineered macrophage-specific promoter of any one of embodiments 40-98, wherein the at least one nucleotide motif comprises a motif having a sequence within the nucleotide sequence of SEQ ID NO: 132, wherein the motif corresponds to position 1257 to position 1275 of SEQ ID NO: 132.
   Embodiment 100: The engineered macrophage-specific promoter of any one of embodiments 40-98, wherein the ablation comprises a nucleotide substitution comprising the sequence TAACATCGTTCTCAGCTA (SEQ ID NO:207) from position 1257 to position 1275 of SEQ ID NO: 132.
   Embodiment 101: The engineered macrophage-specific promoter of any one of embodiments 40-98, wherein the ablation comprises nucleotide deletions of position 1257 to position 1275 of SEQ ID NO: 132.
   Embodiment 102: The engineered macrophage-specific promoter of any one of embodiments 40-101, wherein the at least one nucleotide motif comprises a motif having a sequence within the nucleotide sequence of SEQ ID NO: 132, wherein the motif corresponds to position 1310 to position 1333 of SEQ ID NO: 132.
   Embodiment 103: The engineered macrophage-specific promoter of any one of embodiments 40-101, wherein the ablation comprises a nucleotide substitution comprising the sequence ATATACAGTGTTCAGCGTGTTAC (SEQ ID NO:209) from position 1310 to position 1333 of SEQ ID NO: 132.
   Embodiment 104: The engineered macrophage-specific promoter of any one of embodiments 40-101, wherein the ablation comprises nucleotide deletions of position 1310 to position 1333 of SEQ ID NO: 132.
   Embodiment 105: The engineered macrophage-specific promoter of any one of embodiments 40-104, wherein the at least one nucleotide motif comprises a motif having a sequence within the nucleotide sequence of SEQ ID NO: 132, wherein the motif corresponds to position 1381 to position 1434 of SEQ ID NO: 132.
   Embodiment 106: The engineered macrophage-specific promoter of any one of embodiments 40-104, wherein the ablation comprises a nucleotide substitution comprising the sequence GACGTCTGTTAGTAGTATTACCCGTGTATTTCGGTCTTCGAGCAATTACTTTA (SEQ ID NO:211) from position 1381 to position 1434 of SEQ ID NO: 132.
   Embodiment 107: The engineered macrophage-specific promoter of any one of embodiments 40-104, wherein the ablation comprises nucleotide deletions of position 1381 to position 1434 of SEQ ID NO: 132.
   Embodiment 108: The engineered macrophage-specific promoter of any one of embodiments 40-107, wherein the at least one nucleotide motif comprises a motif having a sequence within the nucleotide sequence of SEQ ID NO: 132, wherein the motif corresponds to position 1698 to position 1753 of SEQ ID NO: 132.
   Embodiment 109: The engineered macrophage-specific promoter of any one of embodiments 40-107, wherein the ablation comprises a nucleotide substitution comprising the sequence GTGCATAAAAAGAAATTCACCACGAGTACCTATCTTGGTCTCGTTTGTTGCACTA (SEQ ID NO:213) from position 1698 to position 1753 of SEQ ID NO: 132.
   Embodiment 110: The engineered macrophage-specific promoter of any one of embodiments 40-107, wherein the ablation comprises nucleotide deletions of position 1698 to position 1753 of SEQ ID NO: 132.
   Embodiment 111: The engineered macrophage-specific promoter of any one of embodiments 40-110, wherein the at least one nucleotide motif comprises a motif having a sequence within the nucleotide sequence of SEQ ID NO: 132, wherein the motif corresponds to position 1783 to position 1826 of SEQ ID NO: 132.
   Embodiment 112: The engineered macrophage-specific promoter of any one of embodiments 40-110, wherein the ablation comprises a nucleotide substitution comprising the sequence AAAAACTACCAACCAGTTATCATTTCTCTGTGTAATATCTGAA (SEQ ID NO:215) from position 1783 to position 1826 of SEQ ID NO: 132.
   Embodiment 113: The engineered macrophage-specific promoter of any one of embodiments 40-110, wherein the ablation comprises nucleotide deletions of position 1783 to position 1826 of SEQ ID NO: 132.
   Embodiment 114: The engineered macrophage-specific promoter of any one of embodiments 40-113, wherein at least one nucleotide motif comprises a motif having a sequence within the nucleotide sequence of SEQ ID NO: 132, wherein the motif corresponds to position 1909 to position 1927 of SEQ ID NO: 132.
   Embodiment 115: The engineered macrophage-specific promoter of any one of embodiments 40-113, wherein the ablation comprises a nucleotide substitution comprising the sequence CGCAGAATATCGATATCT (SEQ ID NO:217) from position 1909 to position 1927 of SEQ ID NO: 132.
   Embodiment 116: The engineered macrophage-specific promoter of any one of embodiments 40-113, wherein the ablation comprises nucleotide deletions of position 1909 to position 1927 of SEQ ID NO: 132.
   Embodiment 117: The engineered macrophage-specific promoter of any one of embodiments 40-116, wherein the at least one nucleotide motif comprises a motif having a sequence within the nucleotide sequence of SEQ ID NO: 132, wherein the motif corresponds to position 1946 to position 1961 of SEQ ID NO: 132.
   Embodiment 118: The engineered macrophage-specific promoter of any one of embodiments 40-116, wherein the ablation comprises a nucleotide substitution comprising the sequence CGAATAGCACCTATA (SEQ ID NO:219) from position 1946 to position 1961 of SEQ ID NO: 132.
   Embodiment 119: The engineered macrophage-specific promoter of any one of embodiments 40-116, wherein the ablation comprises nucleotide deletions of position 1946 to position 1961 of SEQ ID NO: 132.
   Embodiment 120: The engineered macrophage-specific promoter of any one of embodiments 40-119, wherein the ablation comprises an ablation of at least two nucleotide motifs.
   Embodiment 121: The engineered macrophage-specific promoter of any one of embodiments 40-120, wherein the ablation comprises an ablation of at least three nucleotide motifs.
   Embodiment 122: The engineered macrophage-specific promoter of any one of embodiments 40-121, wherein the ablation comprises an ablation of at least four nucleotide motifs.
   Embodiment 123: The engineered macrophage-specific promoter of any one of embodiments 40-122, wherein the ablation comprises an ablation of at least five nucleotide motifs.
   Embodiment 124: The engineered macrophage-specific promoter of embodiment 123, wherein the at least five nucleotide motifs comprise:
i. a nucleotide motif corresponding to position 365 to position 376 of SEQ ID NO: 132;
ii. a nucleotide motif corresponding to position 1169 to position 1192 of SEQ ID NO: 132;
iii. a nucleotide motif corresponding to position 1212 to position 1232 of SEQ ID NO: 132;
iv. a nucleotide motif corresponding to position 1257 to position 1275 of SEQ ID NO: 132; and
v. a nucleotide motif corresponding to position 1381 to position 1434 of SEQ ID NO: 132.
   Embodiment 125: The engineered macrophage-specific promoter of embodiment 124, wherein the ablation comprises a nucleotide substitution comprising the sequence GGTGAATTTTC (SEQ ID NO:183) from position 365 to position 376 of SEQ ID NO: 132.
   Embodiment 126: The engineered macrophage-specific promoter of embodiment 124, wherein the ablation of the nucleotide motif corresponding to position 365 to position 376 of SEQ ID NO: 132 comprises a deletion of the nucleotide motif.
   Embodiment 127: The engineered macrophage-specific promoter of any one of embodiments 124-126, wherein the ablation comprises a nucleotide substitution comprising the sequence CGCGTAGAACTTCGTAACATTAA (SEQ ID NO:203) from position 1169 to position 1192 of SEQ ID NO: 132.
   Embodiment 128: The engineered macrophage-specific promoter of any one of embodiments 124-126, wherein the ablation of the nucleotide motif corresponding to position 1169 to position 1192 of SEQ ID NO:132 comprises a deletion of the nucleotide motif.
   Embodiment 129: The engineered macrophage-specific promoter of any one of embodiments 124-128, wherein the ablation comprises a nucleotide substitution comprising the sequence AGATAACGCCGTCATTGTAT (SEQ ID NO:205) from position 1212 to position 1232 of SEQ ID NO:132.
   Embodiment 130: The engineered macrophage-specific promoter of any one of embodiments 124-128, wherein the ablation of the nucleotide motif corresponding to position 1212 to position 1232 of SEQ ID NO:132 comprises a deletion of the nucleotide motif.
   Embodiment 131: The engineered macrophage-specific promoter of any one of embodiments 124-130, wherein the ablation comprises a nucleotide substitution comprising the sequence TAACATCGTTCTCAGCTA (SEQ ID NO:207) from position 1257 to position 1275 of SEQ ID NO:132.
   Embodiment 132: The engineered macrophage-specific promoter of any one of embodiments 124-130, wherein the ablation of the nucleotide motif corresponding to position 1257 to position 1275 of SEQ ID NO:132 comprises a deletion of the nucleotide motif.
   Embodiment 133: The engineered macrophage-specific promoter of any one of embodiments 124-132, wherein the ablation comprises a nucleotide substitution comprising the sequence GACGTCTGTTAGTAGTATTACCCGTGTATTTCGGTCTTCGAGCAATTACTTTA (SEQ ID NO:211) from position 1381 to position 1434 of SEQ ID NO:132.
   Embodiment 134: The engineered macrophage-specific promoter of any one of embodiments 124-132, wherein the ablation of the nucleotide motif corresponding to position 1381 to position 1434 of SEQ ID NO:132 comprises a deletion of the nucleotide motif.
   Embodiment 135: The engineered macrophage-specific promoter of embodiment 124-134, wherein the engineered macrophage-specific promoter comprises the nucleotide sequence of SEQ ID NO: 123.
   Embodiment 136: The engineered macrophage-specific promoter of embodiment 124-134, wherein the engineered macrophage-specific promoter comprises the nucleotide sequence of SEQ ID NO: 125.
   Embodiment 137: The engineered macrophage-specific promoter of embodiment 124-136, wherein the engineered macrophage-specific promoter further comprises an additional nucleotide motif selected from the group consisting of
i. a sequence corresponding to position 1002 to position 1028 of SEQ ID NO: 132;
ii. a sequence corresponding to position 1310 to position 1333 of SEQ ID NO: 132; and
iii. a sequence corresponding to position 1909 to position 1927 of SEQ ID NO: 132.
   Embodiment 138: The engineered macrophage-specific promoter of any one of embodiments 40-137, wherein the ablation comprises an ablation of at least six nucleotide motifs.
   Embodiment 139: The engineered macrophage-specific promoter of any one of embodiments 40-138, wherein the ablation comprises an ablation of at least seven nucleotide motifs.
   Embodiment 140: The engineered macrophage-specific promoter of any one of embodiments 40-139, wherein the ablation comprises an ablation of at least eight nucleotide motifs.
   Embodiment 141: The engineered macrophage-specific promoter of embodiment 140, wherein the at least eight nucleotide motifs comprise:
i. a nucleotide motif corresponding to position 365 to position 376 of SEQ ID NO: 132;
ii. a nucleotide motif corresponding to position 1169 to position 1192 of SEQ ID NO: 132;
iii. a nucleotide motif corresponding to position 1212 to position 1232 of SEQ ID NO: 132;
iv. a nucleotide motif corresponding to position 1257 to position 1275 of SEQ ID NO: 132; and
v. a nucleotide motif corresponding to position 1381 to position 1434 of SEQ ID NO: 132.
vi. a sequence corresponding to position 1002 to position 1028 of SEQ ID NO: 132;
vii. a sequence corresponding to position 1310 to position 1333 of SEQ ID NO: 132; and
viii. a sequence corresponding to position 1909 to position 1927 of SEQ ID NO: 132.
   Embodiment 142: The engineered macrophage-specific promoter of embodiment 141, wherein the ablation comprises a nucleotide substitution comprising the sequence GGTGAATTTTC (SEQ ID NO:183) from position 365 to position 376 of SEQ ID NO:132.
   Embodiment 143: The engineered macrophage-specific promoter of embodiment 141, wherein the ablation of the nucleotide motif corresponding to position 365 to position 376 of SEQ ID NO:132 comprises a deletion of the nucleotide motif.
   Embodiment 144: The engineered macrophage-specific promoter of any one of embodiments 141-143, wherein the ablation comprises a nucleotide substitution comprising the sequence CGCGTAGAACTTCGTAACATTAA (SEQ ID NO:203) from position 1169 to position 1192 of SEQ ID NO: 132.
   Embodiment 145: The engineered macrophage-specific promoter of any one of embodiments 141-143, wherein the ablation of the nucleotide motif corresponding to position 1169 to position 1192 of SEQ ID NO:132 comprises a deletion of the nucleotide motif.
   Embodiment 146: The engineered macrophage-specific promoter of any one of embodiments 141-145, wherein the ablation comprises a nucleotide substitution comprising the sequence AGATAACGCCGTCATTGTAT (SEQ ID NO:205) from position 1212 to position 1232 of SEQ ID NO: 132.
   Embodiment 147: The engineered macrophage-specific promoter of any one of embodiments 141-145, wherein the ablation of the nucleotide motif corresponding to position 1212 to position 1232 of SEQ ID NO:132 comprises a deletion of the nucleotide motif.
   Embodiment 148: The engineered macrophage-specific promoter of any one of embodiments 141-147, wherein the ablation comprises a nucleotide substitution comprising the sequence TAACATCGTTCTCAGCTA (SEQ ID NO:207) from position 1257 to position 1275 of SEQ ID NO:132.
   Embodiment 149: The engineered macrophage-specific promoter of any one of embodiments 141-147, wherein the ablation of the nucleotide motif corresponding to position 1257 to position 1275 of SEQ ID NO:132 comprises a deletion of the nucleotide motif.
   Embodiment 150: The engineered macrophage-specific promoter of any one of embodiments 141-149, wherein the ablation comprises a nucleotide substitution comprising the sequence GACGTCTGTTAGTAGTATTACCCGTGTATTTCGGTCTTCGAGCAATTACTTTA (SEQ ID NO:211) from position 1381 to position 1434 of SEQ ID NO:132.
   Embodiment 151: The engineered macrophage-specific promoter of any one of embodiments 141-149, wherein the ablation of the nucleotide motif corresponding to position 1381 to position 1434 of SEQ ID NO:132 comprises a deletion of the nucleotide motif.
   Embodiment 152: The engineered macrophage-specific promoter of any one of embodiments 141-151, wherein the ablation comprises a nucleotide substitution comprising the sequence TGAGTAAACTAACTTTCAACCGCTCT (SEQ ID NO:197) from position 1002 to position 1028 of SEQ ID NO:132.
   Embodiment 153: The engineered macrophage-specific promoter of any one of embodiments 141-151, wherein the ablation of the nucleotide motif corresponding to position 1002 to position 1028 of SEQ ID NO:132 comprises a deletion of the nucleotide motif.
   Embodiment 154: The engineered macrophage-specific promoter of any one of embodiments 141-153, wherein the ablation comprises a nucleotide substitution comprising the sequence ATATACAGTGTTCAGCGTGTTAC (SEQ ID NO:209) from position 1310 to position 1333 of SEQ ID NO:132.
   Embodiment 155: The engineered macrophage-specific promoter of any one of embodiments 141-153, wherein the ablation of the nucleotide motif corresponding to position 1310 to position 1333 of SEQ ID NO:132 comprises a deletion of the nucleotide motif.
   Embodiment 156: The engineered macrophage-specific promoter of any one of embodiments 141-155, wherein the ablation comprises a nucleotide substitution comprising the sequence CGCAGAATATCGATATCT (SEQ ID NO:217) from position 1909 to position 1927 of SEQ ID NO:132.
   Embodiment 157: The engineered macrophage-specific promoter of any one of embodiments 141-155, wherein the ablation of the nucleotide motif corresponding to position 1909 to position 1927 of SEQ ID NO:132 comprises a deletion of the nucleotide motif.
   Embodiment 158: The engineered macrophage-specific promoter of embodiment of any one of embodiments 141-157, wherein the engineered macrophage-specific promoter comprises the nucleotide sequence of SEQ ID NO: 124.
   Embodiment 159: The engineered macrophage-specific promoter of any one of embodiments 141-157, wherein the engineered macrophage-specific promoter comprises the nucleotide sequence of SEQ ID NO: 126
   Embodiment 160: The engineered macrophage-specific promoter of embodiment 40 or 41, wherein the wildtype macrophage promoter comprises the nucleotide sequence of SEQ ID NO: 136.
   Embodiment 161: The engineered macrophage-specific promoter according to any one of embodiments 40, 41, and 160, wherein the at least one nucleotide motif comprises a motif having a sequence within the nucleotide sequence of SEQ ID NO: 136, wherein the motif comprises a sequence selected from the group consisting of: to position 133 to position 144 of SEQ ID NO: 136, position 200 to 217 of SEQ ID NO: 136, position 225 to position 247 of SEQ ID NO: 136, position 303 to position 325 of SEQ ID NO: 136, position 332 to position 342 of SEQ ID NO: 136, position 391 to position 413 of SEQ ID NO: 136, position 423 to position 460 of SEQ ID NO: 136, position 467 to position 477 of SEQ ID NO: 136, position 693 to position 717 of SEQ ID NO: 136, position 738 to position 761 of SEQ ID NO: 136, position 838 to position 861 of SEQ ID NO: 136, position 1229 to position 1246 of SEQ ID NO: 136, position 1286 to position 1309 of SEQ ID NO: 136, position 1413 to position 1431 of SEQ ID NO: 136, position 1456 to position 1473 of SEQ ID NO: 136, to position 1530 to position 1544 of SEQ ID NO: 136, position 1577 to position 1590 of SEQ ID NO: 136, position 1816 to position 1836 of SEQ ID NO: 136, position 1852 to position 1872 of SEQ ID NO: 136, and to position 1876 to position to position 1896 of SEQ ID NO: 136.
   Embodiment 162: The engineered macrophage-specific promoter of embodiment 160 or 161, wherein the ablation comprises a substitution or deletion of one or more nucleotides of the at least one nucleotide motif.
   Embodiment 163: The engineered macrophage-specific promoter of any one of embodiments 160-162, wherein the at least one nucleotide motif comprises a motif having a sequence within the nucleotide sequence of SEQ ID NO: 136, wherein the motif corresponds to position 133 to position 144 of SEQ ID NO: 136.
   Embodiment 164: The engineered macrophage-specific promoter of any one of embodiments 160-162, wherein the ablation comprises a nucleotide substitution comprising the sequence CTTACCTACTA (SEQ ID NO: 221) from position 133 to position 144 of SEQ ID NO: 136.
   Embodiment 165: The engineered macrophage-specific promoter of any one of embodiments 160-162, wherein the ablation comprises nucleotide deletions of position 133 to position 144 of SEQ ID NO: 136.
   Embodiment 166: The engineered macrophage-specific promoter of any one of embodiments 160-165, wherein the at least one nucleotide motif comprises a motif having a sequence within the nucleotide sequence of SEQ ID NO: 136, wherein the motif corresponds to position 200 to position 217 of SEQ ID NO: 136.
   Embodiment 167: The engineered macrophage-specific promoter of any one of embodiments 160-165, wherein the ablation comprises a nucleotide substitution comprising the sequence TAATTCGTCCGATAGAT (SEQ ID NO: 223) from position 200 to position 217 of SEQ ID NO: 136.
   Embodiment 168: The engineered macrophage-specific promoter of any one of embodiments 160-165, wherein the ablation comprises nucleotide deletions of position 200 to position 217 of SEQ ID NO: 136.
   Embodiment 169: The engineered macrophage-specific promoter of any one of embodiments 160-168, wherein the at least one nucleotide motif comprises a motif having a sequence within the nucleotide sequence of SEQ ID NO: 136, wherein the motif corresponds to position 225 to position 247 of SEQ ID NO: 136.
   Embodiment 170: The engineered macrophage-specific promoter of any one of embodiments 160-168, wherein the ablation comprises a nucleotide substitution comprising the sequence AATTCAGACGACAAACCATTCT (SEQ ID NO: 225) from position 225 to position 247 of SEQ ID NO: 136.
   Embodiment 171: The engineered macrophage-specific promoter of any one of embodiments 160-168, wherein the ablation comprises nucleotide deletions of position 225 to position 247 of SEQ ID NO: 136.
   Embodiment 172: The engineered macrophage-specific promoter of any one of embodiments 160-171, wherein the at least one nucleotide motif comprises a motif having a sequence within the nucleotide sequence of SEQ ID NO: 136, wherein the motif corresponds to position 303 to position 325 of SEQ ID NO: 136.
   Embodiment 173: The engineered macrophage-specific promoter of any one of embodiments 160-171, wherein the ablation comprises a nucleotide substitution comprising the sequence TTCTAAGTCCAATTCACGACAA (SEQ ID NO: 227) from position 303 to position 325 of SEQ ID NO: 136.
   Embodiment 174: The engineered macrophage-specific promoter of any one of embodiments 160-171, wherein the ablation comprises nucleotide deletions of position 303 to position 325 of SEQ ID NO: 136.
   Embodiment 175: The engineered macrophage-specific promoter of any one of embodiments 160-174, wherein the at least one nucleotide motif comprises a motif having a sequence within the nucleotide sequence of SEQ ID NO: 136, wherein the motif corresponds to position 332 to position 342 of SEQ ID NO: 136.
   Embodiment 176: The engineered macrophage-specific promoter of any one of embodiments 160-174, wherein the ablation comprises a nucleotide substitution comprising the sequence GTTGAAGCTT (SEQ ID NO: 229) from position 332 to position 342 of SEQ ID NO: 136.
   Embodiment 177: The engineered macrophage-specific promoter of any one of embodiments 160-174, wherein the ablation comprises nucleotide deletions of position 332 to position 342 of SEQ ID NO: 136.
   Embodiment 178: The engineered macrophage-specific promoter of any one of embodiments 160-177, wherein the at least one nucleotide motif comprises a motif having a sequence within the nucleotide sequence of SEQ ID NO: 136, wherein the motif corresponds to position 391 to position 413 of SEQ ID NO: 136.
   Embodiment 179: The engineered macrophage-specific promoter of any one of embodiments 160-177, wherein the ablation comprises a nucleotide substitution comprising the sequence AGTCGTCAGACTCAATTATTAC (SEQ ID NO: 231) from position 391 to position 413 of SEQ ID NO: 136.
   Embodiment 180: The engineered macrophage-specific promoter of any one of embodiments 160-177, wherein the ablation comprises nucleotide deletions of position 391 to position 413 of SEQ ID NO: 136.
   Embodiment 181: The engineered macrophage-specific promoter of any one of embodiments 160-180, wherein the at least one nucleotide motif comprises a motif having a sequence within the nucleotide sequence of SEQ ID NO: 136, wherein the motif corresponds to position 423 to position 460 of SEQ ID NO: 136.
   Embodiment 182: The engineered macrophage-specific promoter of any one of embodiments 160-180, wherein the ablation comprises a nucleotide substitution comprising the sequence TCCCTAGCGATCGAAGTTGATAAAACCTAAGTTTTGT (SEQ ID NO: 233) from position 423 to position 460 of SEQ ID NO: 136.
   Embodiment 183: The engineered macrophage-specific promoter of any one of embodiments 160-180, wherein the ablation comprises nucleotide deletions of position 423 to position 460 of SEQ ID NO: 136.
   Embodiment 184: The engineered macrophage-specific promoter of any one of embodiments 160-183, wherein the at least one nucleotide motif comprises a motif having a sequence within the nucleotide sequence of SEQ ID NO: 136, wherein the motif corresponds to position 467 to position 477 of SEQ ID NO: 136.
   Embodiment 185: The engineered macrophage-specific promoter of any one of embodiments 160-183, wherein the ablation comprises a nucleotide substitution comprising the sequence GCCTTCATAA (SEQ ID NO: 235) from position 467 to position 477 of SEQ ID NO: 136.
   Embodiment 186: The engineered macrophage-specific promoter of any one of embodiments 160-183, wherein the ablation comprises nucleotide deletions of position 467 to position 477 of SEQ ID NO: 136.
   Embodiment 187: The engineered macrophage-specific promoter of any one of embodiments 160-186, wherein the at least one nucleotide motif comprises a motif having a sequence within the nucleotide sequence of SEQ ID NO: 136, wherein the motif corresponds to position 693 to position 717 of SEQ ID NO: 136.
   Embodiment 188: The engineered macrophage-specific promoter of any one of embodiments 160-186, wherein the ablation comprises a nucleotide substitution comprising the sequence TCTCGCTAATAGGAGTAAGATACA (SEQ ID NO: 237) from position 693 to position 717 of SEQ ID NO: 136.
   Embodiment 189: The engineered macrophage-specific promoter of any one of embodiments 160-186, wherein the ablation comprises nucleotide deletions of position 693 to position 717 of SEQ ID NO: 136.
   Embodiment 190: The engineered macrophage-specific promoter of any one of embodiments 160-189, wherein the at least one nucleotide motif comprises a motif having a sequence within the nucleotide sequence of SEQ ID NO: 136, wherein the motif corresponds to position 738 to position 761 of SEQ ID NO: 136.
   Embodiment 191: The engineered macrophage-specific promoter of any one of embodiments 160-189, wherein the ablation comprises a nucleotide substitution comprising the sequence TTCTGCTGCAAGACCTATACTAT (SEQ ID NO: 239) from position 738 to position 761 of SEQ ID NO: 136.
   Embodiment 192: The engineered macrophage-specific promoter of any one of embodiments 160-189, wherein the ablation comprises nucleotide deletions of position 738 to position 761 of SEQ ID NO: 136.
   Embodiment 193: The engineered macrophage-specific promoter of any one of embodiments 160-192, wherein the at least one nucleotide motif comprises a motif having a sequence within the nucleotide sequence of SEQ ID NO: 136, wherein the motif corresponds to position 838 to position 861 of SEQ ID NO: 136.
   Embodiment 194: The engineered macrophage-specific promoter of any one of embodiments 160-192, wherein the ablation comprises a nucleotide substitution comprising the sequence CCACATTGCTATAGTGCTGTATA (SEQ ID NO: 241) from position 838 to position 861 of SEQ ID NO: 136.
   Embodiment 195: The engineered macrophage-specific promoter of any one of embodiments 160-192, wherein the ablation comprises nucleotide deletions of position 838 to position 861 of SEQ ID NO: 136.
   Embodiment 196: The engineered macrophage-specific promoter of any one of embodiments 160-195, wherein the at least one nucleotide motif comprises a motif having a sequence within the nucleotide sequence of SEQ ID NO: 136, wherein the motif corresponds to position 1229 to position 1246 of SEQ ID NO: 136.
   Embodiment 197: The engineered macrophage-specific promoter of any one of embodiments 160-195, wherein the ablation comprises a nucleotide substitution comprising the sequence TGCGTACCAGAATATTT (SEQ ID NO: 243) from position 1229 to position 1246 of SEQ ID NO: 136.
   Embodiment 198: The engineered macrophage-specific promoter of any one of embodiments 160-195, wherein the ablation comprises nucleotide deletions of position 1229 to position 1246 of SEQ ID NO: 136.
   Embodiment 199: The engineered macrophage-specific promoter of any one of embodiments 160-198, wherein the at least one nucleotide motif comprises a motif having a sequence within the nucleotide sequence of SEQ ID NO: 136, wherein the motif corresponds to position 1286 to position 1309 of SEQ ID NO: 136.
   Embodiment 200: The engineered macrophage-specific promoter of any one of embodiments 160-198, wherein the ablation comprises a nucleotide substitution comprising the sequence TGGTCACTATCACGTATATACCA (SEQ ID NO: 245) from position 1286 to position 1309 of SEQ ID NO: 136.
   Embodiment 201: The engineered macrophage-specific promoter of any one of embodiments 160-198, wherein the ablation comprises nucleotide deletions of position 1286 to position 1309 of SEQ ID NO: 136.
   Embodiment 202: The engineered macrophage-specific promoter of any one of embodiments 160-201, wherein the at least one nucleotide motif comprises a motif having a sequence within the nucleotide sequence of SEQ ID NO: 136, wherein the motif corresponds to position 1413 to position 1431 of SEQ ID NO: 136.
   Embodiment 203: The engineered macrophage-specific promoter of any one of embodiments 160-201, wherein the ablation comprises a nucleotide substitution comprising the sequence CGAGTTCGATAATACACT (SEQ ID NO: 247) from position 1413 to position 1431 of SEQ ID NO: 136.
   Embodiment 204: The engineered macrophage-specific promoter of any one of embodiments 160-201, wherein the ablation comprises nucleotide deletions of position 1413 to position 1431 of SEQ ID NO: 136.
   Embodiment 205: The engineered macrophage-specific promoter of any one of embodiments 160-204, wherein the at least one nucleotide motif comprises a motif having a sequence within the nucleotide sequence of SEQ ID NO: 136, wherein the motif corresponds to position 1456 to position 1473 of SEQ ID NO: 136.
   Embodiment 206: The engineered macrophage-specific promoter of any one of embodiments 160-204, wherein the ablation comprises a nucleotide substitution comprising the sequence AATACTGGTGCTTCAAT (SEQ ID NO: 249) from position 1456 to position 1473 of SEQ ID NO: 136.
   Embodiment 207: The engineered macrophage-specific promoter of any one of embodiments 160-204, wherein the ablation comprises nucleotide deletions of position 1456 to position 1473 of SEQ ID NO: 136.
   Embodiment 208: The engineered macrophage-specific promoter of any one of embodiments 160-207, wherein the at least one nucleotide motif comprises a motif having a sequence within the nucleotide sequence of SEQ ID NO: 136, wherein the motif corresponds to position 1530 to position 1544 of SEQ ID NO: 136.
   Embodiment 209: The engineered macrophage-specific promoter of any one of embodiments 160-207, wherein the ablation comprises a nucleotide substitution comprising the sequence CCGATAGAAAGAAT (SEQ ID NO: 251) from position 1530 to position 1544 of SEQ ID NO: 136.
   Embodiment 210: The engineered macrophage-specific promoter of any one of embodiments 160-207, wherein the ablation comprises nucleotide deletions of position 1530 to position 1544 of SEQ ID NO: 136.
   Embodiment 211: The engineered macrophage-specific promoter of any one of embodiments 160-210, wherein the at least one nucleotide motif comprises a motif having a sequence within the nucleotide sequence of SEQ ID NO: 136, wherein the motif corresponds to position 1577 to position 1590 of SEQ ID NO: 136.
   Embodiment 212: The engineered macrophage-specific promoter of any one of embodiments 160-210, wherein the ablation comprises a nucleotide substitution comprising the sequence TGTCTGTATAAAG (SEQ ID NO: 253) from position 1577 to position 1590 of SEQ ID NO: 136.
   Embodiment 213: The engineered macrophage-specific promoter of any one of embodiments 160-210, wherein the ablation comprises nucleotide deletions of position 1577 to position 1590 of SEQ ID NO: 136.
   Embodiment 214: The engineered macrophage-specific promoter of any one of embodiments 160-213, wherein the at least one nucleotide motif comprises a motif having a sequence within the nucleotide sequence of SEQ ID NO: 136, wherein the motif corresponds to position 1816 to position 1836 of SEQ ID NO: 136.
   Embodiment 215: The engineered macrophage-specific promoter of any one of embodiments 160-213, wherein the ablation comprises a nucleotide substitution comprising the sequence TGTTAAGCATACTAAACTGT (SEQ ID NO: 255) from position 1816 to position 1836 of SEQ ID NO: 136.
   Embodiment 216: The engineered macrophage-specific promoter of any one of embodiments 160-213, wherein the ablation comprises nucleotide deletions of position 1816 to position 1836 of SEQ ID NO: 136.
   Embodiment 217: The engineered macrophage-specific promoter of any one of embodiments 160-216, wherein the at least one nucleotide motif comprises a motif having a sequence within the nucleotide sequence of SEQ ID NO: 136, wherein the motif corresponds to position 1852 to position 1872 of SEQ ID NO: 136.
   Embodiment 218: The engineered macrophage-specific promoter of any one of embodiments 160-216, wherein the ablation comprises a nucleotide substitution comprising the sequence TTTCGAGCGACGCTTAATAT (SEQ ID NO: 257) from position 1852 to position 1872 of SEQ ID NO: 136.
   Embodiment 219: The engineered macrophage-specific promoter of any one of embodiments 160-216, wherein the ablation comprises nucleotide deletions of position 1852 to position 1872 of SEQ ID NO: 136.
   Embodiment 220: The engineered macrophage-specific promoter of any one of embodiments 160-219, wherein the at least one nucleotide motif comprises a motif having a sequence within the nucleotide sequence of SEQ ID NO: 136, wherein the motif corresponds to position 1876 to position to position 1896 of SEQ ID NO: 136.
   Embodiment 221: The engineered macrophage-specific promoter of any one of embodiments 160-219, wherein the ablation comprises a nucleotide substitution comprising the sequence TAGATAGTACGGGTTCCATA (SEQ ID NO: 259) from position 1876 to position to position 1896 of SEQ ID NO: 136.
   Embodiment 222: The engineered macrophage-specific promoter of any one of embodiments 160-219, wherein the ablation comprises nucleotide deletions of position 1876 to position to position 1896 of SEQ ID NO: 136.
   Embodiment 223: The engineered macrophage-specific promoter of embodiment 40 or 41, wherein the wildtype macrophage promoter comprises the nucleotide sequence of SEQ ID NO: 137.
   Embodiment 224: The engineered macrophage-specific promoter according to any one of embodiments 40, 41, and 223, wherein the at least one nucleotide motif comprises a motif having a sequence within the nucleotide sequence of SEQ ID NO: 137, wherein the motif comprises a sequence selected from the group consisting of: to position 43 to position 60, position 107 to position 120 of SEQ ID NO: 137, position 210 to position 230 of SEQ ID NO: 137, position 345 to position 407 of SEQ ID NO: 137, position 427 to position 457 of SEQ ID NO: 137, position 468 to position 484 of SEQ ID NO: 137, position 560 to position 582, position 730 to position 746 of SEQ ID NO: 137, position 809 to position 820 of SEQ ID NO: ZZZ, position 827 to position 837 of SEQ ID NO: 137, position 858 to position 878 of SEQ ID NO: 137, position 1291 to position 1302 of SEQ ID NO: 137, position 1321 to position 1341 of SEQ ID NO: 137, position 1435 to position 1463 of SEQ ID NO: 137, position 1530 to position 1541 of SEQ ID NO: 137, position 1707 to position 1718 of SEQ ID NO: 137, position 1834 to position 1863 of SEQ ID NO: 137, position 1870 to position 1882 of SEQ ID NO: 137, and to position 1913 to position 1929 of SEQ ID NO: 137.
   Embodiment 225: The engineered macrophage-specific promoter of embodiment 223 or 224, wherein the ablation comprises a substitution or deletion of one or more nucleotides of the at least one nucleotide motif.
   Embodiment 226: The engineered macrophage-specific promoter of any one of embodiments 223-225, wherein the at least one nucleotide motif comprises a motif having a sequence within the nucleotide sequence of SEQ ID NO: 137, wherein the motif corresponds to position 43 to position 60 of SEQ ID NO: 137.
   Embodiment 227: The engineered macrophage-specific promoter of any one of embodiments 223-225, wherein the ablation comprises a nucleotide substitution comprising the sequence CTTACCTACTAGGTTAA (SEQ ID NO: 261) from position 43 to position 60 of SEQ ID NO: 137.
   Embodiment 228: The engineered macrophage-specific promoter of any one of embodiments 223-225, wherein the ablation comprises nucleotide deletions of position 43 to position 60 of SEQ ID NO: 137.
   Embodiment 229: The engineered macrophage-specific promoter of any one of embodiments 223-228, wherein the at least one nucleotide motif comprises a motif having a sequence within the nucleotide sequence of SEQ ID NO: 137, wherein the motif corresponds to position 107 to position 120 of SEQ ID NO: 137.
   Embodiment 230: The engineered macrophage-specific promoter of any one of embodiments 223-228, wherein the ablation comprises a nucleotide substitution comprising the sequence ACTCGAATTCAGA (SEQ ID NO: 263) from position 107 to position 120 of SEQ ID NO: 137.
   Embodiment 231: The engineered macrophage-specific promoter of any one of embodiments 223-228, wherein the ablation comprises nucleotide deletions of position 107 to position 120 of SEQ ID NO: 137.
   Embodiment 232: The engineered macrophage-specific promoter of any one of embodiments 223-231, wherein the at least one nucleotide motif comprises a motif having a sequence within the nucleotide sequence of SEQ ID NO: 137, wherein the motif corresponds to position 210 to position 230 of SEQ ID NO: 137.
   Embodiment 233: The engineered macrophage-specific promoter of any one of embodiments 223-231, wherein the ablation comprises a nucleotide substitution comprising the sequence ATTCTAGCCTTACAGCCTAA (SEQ ID NO: 265) from position 210 to position 230 of SEQ ID NO: 137.
   Embodiment 234: The engineered macrophage-specific promoter of any one of embodiments 223-231, wherein the ablation comprises nucleotide deletions of position 210 to position 230 of SEQ ID NO: 137.
   Embodiment 235: The engineered macrophage-specific promoter of any one of embodiments 223-234, wherein the at least one nucleotide motif comprises a motif having a sequence within the nucleotide sequence of SEQ ID NO: 137, wherein the motif corresponds to position 345 to position 407 of SEQ ID NO: 137.
   Embodiment 236: The engineered macrophage-specific promoter of any one of embodiments 223-234, wherein the ablation comprises a nucleotide substitution comprising the sequence ACTCTACGGAAGTAGCTTGTTTAAAACCTATAGTCTCTTCGGAGTCGTTCTACTA GTACAAA (SEQ ID NO: 267) from position 345 to position 407 of SEQ ID NO: 137.
   Embodiment 237: The engineered macrophage-specific promoter of any one of embodiments 223-234, wherein the ablation comprises nucleotide deletions of position 345 to position 407 of SEQ ID NO: 137.
   Embodiment 238: The engineered macrophage-specific promoter of any one of embodiments 223-237, wherein the at least one nucleotide motif comprises a motif having a sequence within the nucleotide sequence of SEQ ID NO: 137, wherein the motif corresponds to position 427 to position 457 of SEQ ID NO: 137.
   Embodiment 239: The engineered macrophage-specific promoter of any one of embodiments 223-237, wherein the ablation comprises a nucleotide substitution comprising the sequence TGAGTAAACTAACTTTCAACCGCTCTTCGT (SEQ ID NO: 269) from position 427 to position 457 of SEQ ID NO: 137.
   Embodiment 240: The engineered macrophage-specific promoter of any one of embodiments 223-237, wherein the ablation comprises nucleotide deletions of 427 to position 457 of SEQ ID NO: 137.
   Embodiment 241: The engineered macrophage-specific promoter of any one of embodiments 223-240, wherein the at least one nucleotide motif comprises a motif having a sequence within the nucleotide sequence of SEQ ID NO: 137, wherein the motif corresponds to position 468 to position 484 of SEQ ID NO: 137.
   Embodiment 242: The engineered macrophage-specific promoter of any one of embodiments 223-240, wherein the ablation comprises a nucleotide substitution comprising the sequence CTTAAACACCGTTTTG (SEQ ID NO: 271) from position 468 to position 484 of SEQ ID NO: 137.
   Embodiment 243: The engineered macrophage-specific promoter of any one of embodiments 223-240, wherein the ablation comprises nucleotide deletions of position 468 to position 484 of SEQ ID NO: 137.
   Embodiment 244: The engineered macrophage-specific promoter of any one of embodiments 223-243, wherein the at least one nucleotide motif comprises a motif having a sequence within the nucleotide sequence of SEQ ID NO: 137, wherein the motif corresponds to position 560 to position 582 of SEQ ID NO: 137.
   Embodiment 245: The engineered macrophage-specific promoter of any one of embodiments 223-243, wherein the ablation comprises a nucleotide substitution comprising the sequence CTGTAATATCATCCGCTCTTTA (SEQ ID NO: 273) from position 560 to position 582 of SEQ ID NO: 137.
   Embodiment 246: The engineered macrophage-specific promoter of any one of embodiments 223-243, wherein the ablation comprises nucleotide deletions of position 560 to position 582 of SEQ ID NO: 137.
   Embodiment 247: The engineered macrophage-specific promoter of any one of embodiments 223-246, wherein the at least one nucleotide motif comprises a motif having a sequence within the nucleotide sequence of SEQ ID NO: 137, wherein the motif corresponds to position 730 to position 746 of SEQ ID NO: 137.
   Embodiment 248: The engineered macrophage-specific promoter of any one of embodiments 223-246, wherein the ablation comprises a nucleotide substitution comprising the sequence TGATCGGCCAATATTT (SEQ ID NO: 274) from position 730 to position 746 of SEQ ID NO: 137.
   Embodiment 249: The engineered macrophage-specific promoter of any one of embodiments 223-246, wherein the ablation comprises nucleotide deletions of position 730 to position 746 of SEQ ID NO: 137.
   Embodiment 250: The engineered macrophage-specific promoter of any one of embodiments 223-249, wherein the at least one nucleotide motif comprises a motif having a sequence within the nucleotide sequence of SEQ ID NO: 137, wherein the motif corresponds to position 809 to position 820 of SEQ ID NO: 137.
   Embodiment 251: The engineered macrophage-specific promoter of any one of embodiments 223-249, wherein the ablation comprises a nucleotide substitution comprising the sequence TAGAACTTCGT (SEQ ID NO: 276) from position 809 to position 820 of SEQ ID NO: 137.
   Embodiment 252: The engineered macrophage-specific promoter of any one of embodiments 223-249, wherein the ablation comprises nucleotide deletions of position 809 to position 820 of SEQ ID NO: 137.
   Embodiment 253: The engineered macrophage-specific promoter of any one of embodiments 223-252, wherein the at least one nucleotide motif comprises a motif having a sequence within the nucleotide sequence of SEQ ID NO: 137, wherein the motif corresponds to position 827 to position 837 of SEQ ID NO: 137.
   Embodiment 254: The engineered macrophage-specific promoter of any one of embodiments 223-252, wherein the ablation comprises a nucleotide substitution comprising the sequence AACATTAAGT (SEQ ID NO: 278) from position 827 to position 837 of SEQ ID NO: 137.
   Embodiment 255: The engineered macrophage-specific promoter of any one of embodiments 223-252, wherein the ablation comprises nucleotide deletions of position 827 to position 837 of SEQ ID NO: 137.
   Embodiment 256: The engineered macrophage-specific promoter of any one of embodiments 223-255, wherein the at least one nucleotide motif comprises a motif having a sequence within the nucleotide sequence of SEQ ID NO: 137, wherein the motif corresponds to position 858 to position 878 of SEQ ID NO: 137.
   Embodiment 257: The engineered macrophage-specific promoter of any one of embodiments 223-255, wherein the ablation comprises a nucleotide substitution comprising the sequence TAGATAACGCCGTCATTGTA (SEQ ID NO: 280) from position 858 to position 878 of SEQ ID NO: 137.
   Embodiment 258: The engineered macrophage-specific promoter of any one of embodiments 223-255, wherein the ablation comprises nucleotide deletions of position 858 to position 878 of SEQ ID NO: 137.
   Embodiment 259: The engineered macrophage-specific promoter of any one of embodiments 223-258, wherein the at least one nucleotide motif comprises a motif having a sequence within the nucleotide sequence of SEQ ID NO: 137, wherein the motif corresponds to position 1291 to position 1302 of SEQ ID NO: 137.
   Embodiment 260: The engineered macrophage-specific promoter of any one of embodiments 223-258, wherein the ablation comprises a nucleotide substitution comprising the sequence TTTCTCTAACG (SEQ ID NO: 282) from position 1291 to position 1302 of SEQ ID NO: 137.
   Embodiment 261: The engineered macrophage-specific promoter of any one of embodiments 223-258, wherein the ablation comprises nucleotide deletions of position 1291 to position 1302 of SEQ ID NO: 137.
   Embodiment 262: The engineered macrophage-specific promoter of any one of embodiments 223-261, wherein the at least one nucleotide motif comprises a motif having a sequence within the nucleotide sequence of SEQ ID NO: 137, wherein the motif corresponds to position 1321 to position 1341 of SEQ ID NO: 137.
   Embodiment 263: The engineered macrophage-specific promoter of any one of embodiments 223-261, wherein the ablation comprises a nucleotide substitution comprising the sequence CTAACATCGTTCTCAGCTAA (SEQ ID NO: 284) from position 1321 to position 1341 of SEQ ID NO: 137.
   Embodiment 264: The engineered macrophage-specific promoter of any one of embodiments 223-261, wherein the ablation comprises nucleotide deletions of position 1321 to position 1341 of SEQ ID NO: 137.
   Embodiment 265: The engineered macrophage-specific promoter of any one of embodiments 223-264, wherein the at least one nucleotide motif comprises a motif having a sequence within the nucleotide sequence of SEQ ID NO: 137, wherein the motif corresponds to position 1435 to position 1463 of SEQ ID NO: 137.
   Embodiment 266: The engineered macrophage-specific promoter of any one of embodiments 223-264, wherein the ablation comprises a nucleotide substitution comprising the sequence TATACAGTGTTCAGCGTGTTACTTGTGA (SEQ ID NO: 286) from position 1435 to position 1463 of SEQ ID NO: 137.
   Embodiment 267: The engineered macrophage-specific promoter of any one of embodiments 223-264, wherein the ablation comprises nucleotide deletions of position 1435 to position 1463 of SEQ ID NO: 137.
   Embodiment 268: The engineered macrophage-specific promoter of any one of embodiments 223-267, wherein the at least one nucleotide motif comprises a motif having a sequence within the nucleotide sequence of SEQ ID NO: 137, wherein the motif corresponds to position 1530 to position 1541 of SEQ ID NO: 137.
   Embodiment 269: The engineered macrophage-specific promoter of any one of embodiments 223-267, wherein the ablation comprises a nucleotide substitution comprising the sequence CGTACAAGTAT (SEQ ID NO: 288) from position 1530 to position 1541 of SEQ ID NO: 137.
   Embodiment 270: The engineered macrophage-specific promoter of any one of embodiments 223-267, wherein the ablation comprises nucleotide deletions of position 1530 to position 1541 of SEQ ID NO: 137.
   Embodiment 271: The engineered macrophage-specific promoter of any one of embodiments 223-270, wherein the at least one nucleotide motif comprises a motif having a sequence within the nucleotide sequence of SEQ ID NO: 137, wherein the motif corresponds to position 1707 to position 1718 of SEQ ID NO: 137.
   Embodiment 272: The engineered macrophage-specific promoter of any one of embodiments 223-270, wherein the ablation comprises a nucleotide substitution comprising the sequence AGTCTCTGAAT (SEQ ID NO: 290) from position 1707 to position 1718 of SEQ ID NO: 137.
   Embodiment 273: The engineered macrophage-specific promoter of any one of embodiments 223-270, wherein the ablation comprises nucleotide deletions of position 1707 to position 1718 of SEQ ID NO: 137.
   Embodiment 274: The engineered macrophage-specific promoter of any one of embodiments 223-273, wherein the at least one nucleotide motif comprises a motif having a sequence within the nucleotide sequence of SEQ ID NO: 137, wherein the motif corresponds to position 1834 to position 1863 of SEQ ID NO: 137.
   Embodiment 275: The engineered macrophage-specific promoter of any one of embodiments 223-273, wherein the ablation comprises a nucleotide substitution comprising the sequence CCCTATATAATACCCGCTAGCATACAAAT (SEQ ID NO: 292) from position 1834 to position 1863 of SEQ ID NO: 137.
   Embodiment 276: The engineered macrophage-specific promoter of any one of embodiments 223-273, wherein the ablation comprises nucleotide deletions of position 1834 to position 1863 of SEQ ID NO: 137.
   Embodiment 277: The engineered macrophage-specific promoter of any one of embodiments 223-276, wherein the at least one nucleotide motif comprises a motif having a sequence within the nucleotide sequence of SEQ ID NO: 137, wherein the motif corresponds to position 1870 to position 1882 of SEQ ID NO: 137.
   Embodiment 278: The engineered macrophage-specific promoter of any one of embodiments 223-276, wherein the ablation comprises a nucleotide substitution comprising the sequence GTTGCTCATATA (SEQ ID NO: 294) from position 1870 to position 1882of SEQ ID NO: 137.
   Embodiment 279: The engineered macrophage-specific promoter of any one of embodiments 223-276, wherein the ablation comprises nucleotide deletions of position 1870 to position 1882 of SEQ ID NO: 137.
   Embodiment 280: The engineered macrophage-specific promoter of any one of embodiments 223-279, wherein the at least one nucleotide motif comprises a motif having a sequence within the nucleotide sequence of SEQ ID NO: 137, wherein the motif corresponds to position 1913 to position 1929 of SEQ ID NO: 137.
   Embodiment 281: The engineered macrophage-specific promoter of any one of embodiments 223-279, wherein the ablation comprises a nucleotide substitution comprising the sequence ACGTCTGTTAGTAGTA (SEQ ID NO: 296) from position 1913 to position 1929 of SEQ ID NO: 137.
   Embodiment 282: The engineered macrophage-specific promoter of any one of embodiments 223-279, wherein the ablation comprises nucleotide deletions of position 1913 to position 1929 of SEQ ID NO: 137.
   Embodiment 283: An engineered macrophage-specific promoter comprising an ablation of at least one nucleotide motif, wherein the ablation increases specific activity of the engineered macrophage-specific promoter in M2 macrophages, as compared to activity of a corresponding macrophage-specific promoter lacking the ablation in M2 macrophages.
   Embodiment 284: The engineered macrophage-specific promoter of embodiment 283, wherein the corresponding macrophage-specific promoter lacking the ablation in M1 macrophages is a wildtype macrophage promoter, and wherein the wildtype macrophage promoter comprises a sequence selected from the group consisting of SEQ ID NOs 139-141, 392, and 393.
   Embodiment 285: An engineered macrophage-specific promoter comprising at least one regulatory element, wherein the regulatory element exhibits greater activity in an M1 macrophage compared to an M2 or M0 macrophage or exhibits greater activity in an M2 macrophage compared to an M1 or M0 macrophage.
   Embodiment 286: The engineered macrophage-specific promoter of embodiment 285, wherein the engineered macrophage-specific promoter comprises at least 2, at least 3, at least 4, or at least 5 regulatory elements.
   Embodiment 287: The engineered macrophage-specific promoter of any one of embodiments 285 or 279, wherein the engineered macrophage-specific promoter comprises at least 5 regulatory elements.
   Embodiment 288: The engineered macrophage-specific promoter of any one of embodiments 285-287, wherein each of the at least 5 regulatory elements are different.
   Embodiment 289: The engineered macrophage-specific promoter of any one of embodiments 285-287, wherein each of the at least 5 regulatory elements are the same.
   Embodiment 290: The engineered macrophage-specific promoter of any one of embodiments 285-289, wherein the engineered macrophage-specific promoter exhibits increased activity in M1 macrophages compared to M2 macrophages.
   Embodiment 291: The engineered macrophage-specific promoter of any one of embodiments 285-290, wherein the at least one regulatory element comprises a nucleotide sequence selected from the group consisting of SEQ ID NOs: 297-313 and SEQ ID NOs: 372-390.
   Embodiment 292: The engineered macrophage-specific promoter of any one of embodiments 285-290, wherein the at least one regulatory element comprises a nucleotide sequence selected from:
(i) a nucleotide sequence having at least 75%, at least 80%, at least 85%, at least 90%, at least 95%, at least 96%, at least 97%, at least 98%, at least 99% or 100% sequence identity to SEQ ID NO: 440,
(ii) a nucleotide sequence having at least 75%, at least 80%, at least 85%, at least 90%, at least 95%, at least 96%, at least 97%, at least 98%, at least 99% or 100% sequence identity to SEQ ID NO: 441,
(iii) a nucleotide sequence having at least 75%, at least 80%, at least 85%, at least 90%, at least 95%, at least 96%, at least 97%, at least 98%, at least 99% or 100% sequence identity to SEQ ID NO: 442, and
(iv) a nucleotide sequence having at least 75%, at least 80%, at least 85%, at least 90%, at least 95%, at least 96%, at least 97%, at least 98%, at least 99% or 100% sequence identity to SEQ ID NO: 443.
   Embodiment 293: The engineered macrophage-specific promoter of any one of embodiments 285-289, wherein the engineered macrophage-specific promoter exhibits increased activity in M2 macrophages compared to M1 macrophages, M0 macrophages, or both M1 and M0 macrophages.
   Embodiment 294: The engineered macrophage-specific promoter of any one of embodiments 285-289, and 293, wherein the at least one regulatory element comprises a nucleotide sequence selected from the group consisting of SEQ ID NOs: 314-371.
   Embodiment 295: The engineered macrophage-specific promoter of any one of embodiments 285-289, and 293, wherein the at least one regulatory element comprises a nucleotide sequence selected from:
(i) a nucleotide sequence having at least 75%, at least 80%, at least 85%, at least 90%, at least 95%, at least 96%, at least 97%, at least 98%, at least 99% or 100% sequence identity to SEQ ID NO: 420,
(ii) a nucleotide sequence having at least 75%, at least 80%, at least 85%, at least 90%, at least 95%, at least 96%, at least 97%, at least 98%, at least 99% or 100% sequence identity to SEQ ID NO: 421,
(iii) a nucleotide sequence having at least 75%, at least 80%, at least 85%, at least 90%, at least 95%, at least 96%, at least 97%, at least 98%, at least 99% or 100% sequence identity to SEQ ID NO: 422,
(iv) a nucleotide sequence having at least 75%, at least 80%, at least 85%, at least 90%, at least 95%, at least 96%, at least 97%, at least 98%, at least 99% or 100% sequence identity to SEQ ID NO: 423,
(v) a nucleotide sequence having at least 75%, at least 80%, at least 85%, at least 90%, at least 95%, at least 96%, at least 97%, at least 98%, at least 99% or 100% sequence identity to SEQ ID NO: 424,
(vi) a nucleotide sequence having at least 75%, at least 80%, at least 85%, at least 90%, at least 95%, at least 96%, at least 97%, at least 98%, at least 99% or 100% sequence identity to SEQ ID NO: 425,
(vii) a nucleotide sequence having at least 75%, at least 80%, at least 85%, at least 90%, at least 95%, at least 96%, at least 97%, at least 98%, at least 99% or 100% sequence identity to SEQ ID NO: 426,
(viii) a nucleotide sequence having at least 75%, at least 80%, at least 85%, at least 90%, at least 95%, at least 96%, at least 97%, at least 98%, at least 99% or 100% sequence identity to SEQ ID NO: 427,
(ix) a nucleotide sequence having at least 75%, at least 80%, at least 85%, at least 90%, at least 95%, at least 96%, at least 97%, at least 98%, at least 99% or 100% sequence identity to SEQ ID NO: 428,
(x) a nucleotide sequence having at least 75%, at least 80%, at least 85%, at least 90%, at least 95%, at least 96%, at least 97%, at least 98%, at least 99% or 100% sequence identity to SEQ ID NO: 429,
(xi) a nucleotide sequence having at least 75%, at least 80%, at least 85%, at least 90%, at least 95%, at least 96%, at least 97%, at least 98%, at least 99% or 100% sequence identity to SEQ ID NO: 430,
(xii) a nucleotide sequence having at least 75%, at least 80%, at least 85%, at least 90%, at least 95%, at least 96%, at least 97%, at least 98%, at least 99% or 100% sequence identity to SEQ ID NO: 431,
(xiii) a nucleotide sequence having at least 75%, at least 80%, at least 85%, at least 90%, at least 95%, at least 96%, at least 97%, at least 98%, at least 99% or 100% sequence identity to SEQ ID NO: 432,
(xiv) a nucleotide sequence having at least 75%, at least 80%, at least 85%, at least 90%, at least 95%, at least 96%, at least 97%, at least 98%, at least 99% or 100% sequence identity to SEQ ID NO: 433,
(xv) a nucleotide sequence having at least 75%, at least 80%, at least 85%, at least 90%, at least 95%, at least 96%, at least 97%, at least 98%, at least 99% or 100% sequence identity to SEQ ID NO: 434,
(xvi) a nucleotide sequence having at least 75%, at least 80%, at least 85%, at least 90%, at least 95%, at least 96%, at least 97%, at least 98%, at least 99% or 100% sequence identity to SEQ ID NO: 435,
(xvii) a nucleotide sequence having at least 75%, at least 80%, at least 85%, at least 90%, at least 95%, at least 96%, at least 97%, at least 98%, at least 99% or 100% sequence identity to SEQ ID NO: 436,
(xviii) a nucleotide sequence having at least 75%, at least 80%, at least 85%, at least 90%, at least 95%, at least 96%, at least 97%, at least 98%, at least 99% or 100% sequence identity to SEQ ID NO: 437,
(xix) a nucleotide sequence having at least 75%, at least 80%, at least 85%, at least 90%, at least 95%, at least 96%, at least 97%, at least 98%, at least 99% or 100% sequence identity to SEQ ID NO: 438, and
(xx) a nucleotide sequence having at least 75%, at least 80%, at least 85%, at least 90%, at least 95%, at least 96%, at least 97%, at least 98%, at least 99% or 100% sequence identity to SEQ ID NO: 439.
   Embodiment 296: The engineered macrophage-specific promoter of any one of embodiments 285-295, further comprising a minimal promoter operably linked to the engineered macrophage-specific promoter.
   Embodiment 297: The engineered macrophage-specific promoter of embodiment 296, wherein the minimal promoter is derived from a promoter selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, inducer molecule responsive promoters, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, hUBIb, and tandem repeats thereof.
   Embodiment 298: The engineered macrophage-specific promoter of any one of embodiments 1-297, further comprises at least one inert sequence.
   Embodiment 299: The engineered macrophage-specific promoter of embodiment 298, wherein the inert sequence is derived from an insulating element.
   Embodiment 300: The engineered macrophage-specific promoter of any one of embodiments 1-299, further comprising at least one molecular barcode.
   Embodiment 301: The engineered macrophage-specific promoter of any one of embodiments 1-300, wherein M2 macrophages are selected from the group consisting of M2a macrophages, M2b macrophages, and M2c macrophages.
   Embodiment 302: An engineered macrophage-specific promoter system comprising at least one regulatory element and a heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 132.
   Embodiment 303: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 133.
   Embodiment 304: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 134.
   Embodiment 305: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 135.
   Embodiment 306: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 136.
   Embodiment 307: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 137.
   Embodiment 308: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 138.
   Embodiment 309: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 142.
   Embodiment 310: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 143.
   Embodiment 311: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 144.
   Embodiment 312: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 145.
   Embodiment 313: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 146.
   Embodiment 314: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 147.
   Embodiment 315: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 316: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 149.
   Embodiment 317: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 150.
   Embodiment 318: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 151.
   Embodiment 319: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 152.
   Embodiment 320: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 153.
   Embodiment 321: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 154.
   Embodiment 322: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 155.
   Embodiment 323: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 156.
   Embodiment 324: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 157.
   Embodiment 325: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 158.
   Embodiment 326: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 159.
   Embodiment 327: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 160.
   Embodiment 328: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 161.
   Embodiment 329: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 162.
   Embodiment 330: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 331: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 1.
   Embodiment 332: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 2.
   Embodiment 333: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 3.
   Embodiment 334: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 4.
   Embodiment 335: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 5.
   Embodiment 336: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 6.
   Embodiment 337: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 7.
   Embodiment 338: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 8.
   Embodiment 339: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 9.
   Embodiment 340: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 10.
   Embodiment 341: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 11.
   Embodiment 342: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 12.
   Embodiment 343: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 13.
   Embodiment 344: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 14.
   Embodiment 345: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 346: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 16.
   Embodiment 347: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 17.
   Embodiment 348: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 18.
   Embodiment 349: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 19.
   Embodiment 350: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 20.
   Embodiment 351: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 21.
   Embodiment 352: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 22.
   Embodiment 353: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 23.
   Embodiment 354: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 24.
   Embodiment 355: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 25.
   Embodiment 356: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 26.
   Embodiment 357: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 27.
   Embodiment 358: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 28.
   Embodiment 359: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 29.
   Embodiment 360: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 361: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 81.
   Embodiment 362: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 82.
   Embodiment 363: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 88.
   Embodiment 364: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 89.
   Embodiment 365: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 90.
   Embodiment 366: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 91.
   Embodiment 367: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 92.
   Embodiment 368: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 96.
   Embodiment 369: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 97.
   Embodiment 370: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 119.
   Embodiment 371: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 120.
   Embodiment 372: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 121.
   Embodiment 373: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 122.
   Embodiment 374: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 297.
   Embodiment 375: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 376: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 299.
   Embodiment 377: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 300.
   Embodiment 378: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 301.
   Embodiment 379: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 302.
   Embodiment 380: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 303.
   Embodiment 381: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 304.
   Embodiment 382: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 305.
   Embodiment 383: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 306.
   Embodiment 384: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 307.
   Embodiment 385: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 308.
   Embodiment 386: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 309.
   Embodiment 387: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 310.
   Embodiment 388: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 311.
   Embodiment 389: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 312.
   Embodiment 390: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 391: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 372.
   Embodiment 392: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 373.
   Embodiment 393: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 374.
   Embodiment 394: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 375.
   Embodiment 395: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 376.
   Embodiment 396: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 377.
   Embodiment 397: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 378.
   Embodiment 398: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 379.
   Embodiment 399: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 380.
   Embodiment 400: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 381.
   Embodiment 401: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 382.
   Embodiment 402: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 383.
   Embodiment 403: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 384.
   Embodiment 404: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 385.
   Embodiment 405: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 406: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 387.
   Embodiment 407: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 388.
   Embodiment 408: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 389.
   Embodiment 409: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 390.
   Embodiment 410: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 139.
   Embodiment 411: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 140.
   Embodiment 412: An engineered macrophage-specific promoter system comprising at least one regulatory element and at least one heterologous payload, wherein the at least one regulatory element comprises a sequence having at least 75%, at least 80%, at least 85%, at least 90%, at least 95%, at least 96%, at least 97%, at least 98%, at least 99% or 100% sequence identity to SEQ ID NO: 141.
   Embodiment 413: An engineered macrophage-specific promoter system comprising at least one regulatory element and at least one heterologous payload, comprising a nucleotide sequence having at least 75%, at least 80%, at least 85%, at least 90%, at least 95%, at least 96%, at least 97%, at least 98%, at least 99% or 100% sequence identity to SEQ ID NO: 392.
   Embodiment 414: An engineered macrophage-specific promoter system comprising at least one regulatory element and at least one heterologous payload, comprising a nucleotide sequence having at least 75%, at least 80%, at least 85%, at least 90%, at least 95%, at least 96%, at least 97%, at least 98%, at least 99% or 100% sequence identity to SEQ ID NO: 393.
   Embodiment 415: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 314.
   Embodiment 416: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 315.
   Embodiment 417: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 316.
   Embodiment 418: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 317.
   Embodiment 419: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 318.
   Embodiment 420: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 319.
   Embodiment 421: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 320.
   Embodiment 422: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 321.
   Embodiment 423: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 322.
   Embodiment 424: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 323.
   Embodiment 425: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 324.
   Embodiment 426: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 325.
   Embodiment 427: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 428: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 327.
   Embodiment 429: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 328.
   Embodiment 430: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 329.
   Embodiment 431: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 330.
   Embodiment 432: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 331.
   Embodiment 433: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 332.
   Embodiment 434: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 333.
   Embodiment 435: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 334.
   Embodiment 436: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 335.
   Embodiment 437: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 336.
   Embodiment 438: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 337.
   Embodiment 439: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 338.
   Embodiment 440: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 339.
   Embodiment 441: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 340.
   Embodiment 442: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 443: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 342.
   Embodiment 444: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 343.
   Embodiment 445: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 344.
   Embodiment 446: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 345.
   Embodiment 447: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 346.
   Embodiment 448: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 347.
   Embodiment 449: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 348.
   Embodiment 450: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 349.
   Embodiment 451: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 350.
   Embodiment 452: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 351.
   Embodiment 453: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 352.
   Embodiment 454: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 353.
   Embodiment 455: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 354.
   Embodiment 456: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 355.
   Embodiment 457: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 458: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 357.
   Embodiment 459: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 358.
   Embodiment 460: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 359.
   Embodiment 461: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 360.
   Embodiment 462: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 361.
   Embodiment 463: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 362.
   Embodiment 464: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 363.
   Embodiment 465: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 364.
   Embodiment 466: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 365.
   Embodiment 467: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 366.
   Embodiment 468: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 367.
   Embodiment 469: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 368.
   Embodiment 470: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 369.
   Embodiment 471: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 370.
   Embodiment 472: An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or
   Embodiment 473: A heterologous construct comprising
i. the engineered macrophage-specific promoter system of any one of embodiments 1-39, 302-308, or 568-586; or
ii. the engineered macrophage-specific promoter of any one of embodiments 40-301, 309-472, or 548-586 operably linked to a heterologous payload, optionally wherein the heterologous payload is a polynucleotide comprising a nucleotide sequence encoding a polypeptide.
   Embodiment 474: The heterologous construct of embodiment 473, wherein the polypeptide comprises at least one effector molecule.
   Embodiment 475: The heterologous construct of embodiment 473 or 474, wherein the polypeptide comprises a first effector molecule and a second effector molecule.
   Embodiment 476: The heterologous construct of any one of embodiments 473-475, wherein the polynucleotide comprises a nucleotide sequence encoding the first effector molecule, a linker nucleotide sequence, and a nucleotide sequence encoding the second effector.
   Embodiment 477: The heterologous construct according to embodiment 476, wherein the linker nucleotide sequence encodes one or more 2A ribosome skipping elements.
   Embodiment 478: The heterologous construct according to embodiment 477, wherein the one or more 2A ribosome skipping elements comprise elements that are each selected from the group consisting of: P2A, T2A, E2A, and F2A.
   Embodiment 479: The heterologous construct of any one of embodiments 473-478, wherein the at least one effector molecule or each effector molecule is selected from a therapeutic class, wherein the therapeutic class is selected from the group consisting of: a cytokine, a chemokine, a homing molecule, a growth factor, a polynucleotide molecule, a co-activation molecule, a tumor microenvironment modifier, a receptor, a ligand, a transcription factor, an antibody, a peptide, and an enzyme.
   Embodiment 480: The heterologous construct of embodiment 479, wherein the transcription factor is a master regulator.
   Embodiment 481: The heterologous construct of embodiment 479, wherein the transcription factor is a master regulator of polarization to an M1 macrophage.
   Embodiment 482: The heterologous construct of embodiment 481, wherein the transcription factor is IRF7 or a derivative thereof, or p65/RelA or a derivative thereof.
   Embodiment 483: The heterologous construct of embodiment 482, wherein the transcription factor is IRF7 or a derivative thereof, optionally wherein the transcription factor comprises an amino acid sequence having at least 75%, at least 80%, at least 85%, at least 90%, at least 95%, at least 96%, at least 97%, at least 98%, at least 99% or 100% sequence identity to SEQ ID NO: 401, or wherein the amino acid sequence of the transcription factor is SEQ ID NO: 401.
   Embodiment 484: The heterologous construct of embodiment 482, wherein the transcription factor is p65/RelA or a derivative thereof, optionally wherein the transcription factor comprises an amino acid sequence having at least 75%, at least 80%, at least 85%, at least 90%, at least 95%, at least 96%, at least 97%, at least 98%, at least 99% or 100% sequence identity to SEQ ID NO: 403, or wherein the amino acid sequence of the transcription factor is SEQ ID NO: 403.
   Embodiment 485: The heterologous construct of embodiment 479, wherein the transcription factor is a master regulator of polarization to an M2 macrophage.
   Embodiment 486: The heterologous construct of embodiment 479, wherein the at least one effector molecule or each effector molecule is a cytokine.
   Embodiment 487: The heterologous construct of embodiment 486, wherein the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.
   Embodiment 488: The heterologous construct of any one of embodiments 486-487, wherein the cytokine is a master regulator of polarization to an M1 macrophage.
   Embodiment 489: The heterologous construct of embodiment 488, wherein the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof.
   Embodiment 490: The heterologous construct of embodiment 489, wherein the cytokine is IFN-γ or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 395, or wherein the amino acid sequence of the transcription factor is SEQ ID NO: 395.
   Embodiment 491: The heterologous construct of embodiment 489, wherein the cytokine is TNF-α or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 397, or wherein the amino acid sequence of the transcription factor is SEQ ID NO: 397.
   Embodiment 492: The heterologous construct of embodiment 489, wherein the cytokine is IL-12, an IL12p70 fusion protein, or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 399, or wherein the amino acid sequence of the transcription factor is SEQ ID NO: 399.
   Embodiment 493: The heterologous construct of any one of embodiments 486-487, wherein the cytokine is a master regulator of polarization to an M2 macrophage.
   Embodiment 494: The heterologous construct of embodiment 493, wherein the cytokine is IL-10, IL-4, IL-13, IL-21, TGF-beta, M-CSF, or a derivative thereof.
   Embodiment 495: The heterologous construct of embodiment 494, wherein the cytokine is IL-10 or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 405, or wherein the amino acid sequence of the transcription factor is SEQ ID NO: 405.
   Embodiment 496: The heterologous construct of embodiment 494, wherein the cytokine is IL-4 or a derivative thereof, optionally wherein the cytokine comprises an amino acid sequence having at least 75%, at least 80%, at least 85%, at least 90%, at least 95%, at least 96%, at least 97%, at least 98%, at least 99% or 100% sequence identity to SEQ ID NO: 407, or wherein the amino acid sequence of the transcription factor is SEQ ID NO: 407.
   Embodiment 497: The heterologous construct of embodiment 479, wherein the at least one effector molecule or each effector molecule is a chemokine.
   Embodiment 498: The heterologous construct according to embodiment 497, wherein the chemokine is selected from the group consisting of: CCL21a, CXCL10, CXCL11, CXCL13, a CXCL10-CXCL11 fusion protein, CCL19, CXCL9, and CXCL1.
   Embodiment 499: The heterologous construct of embodiment 479, wherein the at least one effector molecule or each effector molecule is a homing molecule.
   Embodiment 500: The heterologous construct according to embodiment 499, wherein the homing molecule is selected from the group consisting of: anti-integrin alpha4, beta7; anti-MAdCAM; CCR9; CXCR4; SDF1; MMP-2; CXCR1; CXCR7; CCR2; CCR4; and GPR15.
   Embodiment 501: The heterologous construct of embodiment 479, wherein the at least one effector molecule or each effector molecule is a growth factor.
   Embodiment 502: The heterologous construct according to embodiment 501, wherein the growth factor is selected from the group consisting of: FLT3L and GM-CSF.
   Embodiment 503: The heterologous construct of embodiment 479, wherein the at least one effector molecule or each effector molecule is a co-activation molecule.
   Embodiment 504: The heterologous construct according to embodiment 503, wherein the co-activation molecule is selected from the group consisting of: c-Jun, 4-1BBL and CD40L.
   Embodiment 505: The heterologous construct of embodiment 479, wherein the at least one effector molecule or each effector molecule is or comprises a tumor microenvironment modifier.
   Embodiment 506: The heterologous construct according to embodiment 505, wherein the tumor microenvironment modifier is selected from the group consisting of: an adenosine deaminase, a TGFbeta inhibitor, an immune checkpoint inhibitor, a VEGF inhibitor, and an HPGE2.
   Embodiment 507: The heterologous construct according to any one of embodiments 475-506, wherein each of the first effector molecule and the second effector molecule are from separate therapeutic classes.
   Embodiment 508: The heterologous construct according to any one of embodiments 475
507, wherein each effector molecule is a human-derived effector molecule.
   Embodiment 509: A heterologous construct for inducing a macrophage to transition from an M1 state to an M2 state, comprising:
a) either (i) the regulatory element derived from a promoter of a gene that is more highly expressed in M1 macrophage compared to M2 or M0 macrophages, as applied to any one of embodiments 1-18, or (ii) the engineered macrophage-specific promoter of any one of embodiments 40-282, 285-292, and 296-409; and
b) a heterologous payload encoding a master regulator of polarization to an M2 macrophage, wherein the regulatory element or engineered macrophage-specific promoter of (a) is operably linked to the heterologous payload and configured to induce expression of the heterologous payload.
   Embodiment 510: The heterologous construct of embodiment 509, wherein the master regulator of polarization to an M2 macrophage is IL-10, IL-4, IL-13, IL-21, TGF-beta, M-CSF, or a derivative thereof.
   Embodiment 511: The heterologous construct of embodiment 510, wherein the master regulator of polarization to an M2 macrophage is IL-10.
   Embodiment 512: The heterologous construct of any one of embodiments 509-511, wherein (a) is a regulatory element derived from a CCL19 promoter, optionally comprising the nucleotide sequence of SEQ ID NO: 132.
   Embodiment 513: The heterologous construct of any one of embodiments 509-512, wherein the M2 state is an M2c state, an M2a state, or an M2b state.
   Embodiment 514: A heterologous construct for stabilizing a macrophage in an M1 polarization state, comprising:
a) either (i) the regulatory element derived from a promoter of a gene that is more highly expressed in M1 macrophage compared to M2 or M0 macrophages, as applied to any one of embodiments 1-18, or (ii) the engineered macrophage-specific promoter of any one of embodiments 40-282, 285-292, and 296-409; and
b) a heterologous payload encoding a master regulator of polarization to an M1 macrophage,
wherein the regulatory element or engineered macrophage-specific promoter of (a) is operably linked to the heterologous payload and configured to induce expression of the heterologous payload.
   Embodiment 515: The heterologous construct of embodiment 514, wherein the master regulator of polarization to an M1 macrophage is a cytokine.
   Embodiment 516: The heterologous construct of embodiment 515, wherein the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof.
   Embodiment 517: The heterologous construct of embodiment 516, wherein the cytokine is IFN-γ or a derivative thereof.
   Embodiment 518: The heterologous construct of embodiment 514, wherein the master regulator of polarization to an M1 macrophage is a transcription factor selected from IRF7 or a derivative thereof, or p65/RelA or a derivative thereof.
   Embodiment 519: The heterologous construct of any one of embodiments 514-518, wherein (a) is a regulatory element derived from a UBD1 promoter, an IDO1 promoter, or a CCL19 promoter.
   Embodiment 520: The heterologous construct of embodiment 519, wherein:
(i) (a) is a regulatory element derived from a UBD1 promoter, optionally wherein the regulatory element derived from the UBD1 promoter comprises the sequence of SEQ ID NO: 137.
(ii) (a) is a regulatory element derived from an IDO1 promoter, optionally wherein the regulatory element derived from the IDO1 promoter comprises the sequence of SEQ ID NO: 136:
(iii) (a) is a regulatory element derived from a CCL19 promoter, optionally wherein the regulatory element derived from the CCL19 promoter comprises the sequence of SEQ ID NO: 123 or 125.
   Embodiment 521: A heterologous construct for inducing a macrophage to transition from an M2 state to an M1 state, comprising:
a) either (i) the regulatory element derived from a promoter of a gene that is more highly expressed in M2 macrophage compared to M1 or M0 macrophages, as applied to any one of embodiments 19-39, or (ii) the engineered macrophage-specific promoter of any one of embodiments 283-289, 293-301, 410-472, and 548-567; and
b) a heterologous payload encoding a master regulator of polarization to an M1 macrophage, wherein the regulatory element or engineered macrophage-specific promoter of (a) is operably linked to the heterologous payload and configured to induce expression of the heterologous payload.
   Embodiment 522: The heterologous construct of embodiment 521, wherein the master regulator of polarization to an M1 macrophage is a cytokine.
   Embodiment 523: The heterologous construct of embodiment 522, wherein the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof.
   Embodiment 524: The heterologous construct of embodiment 521, wherein the master regulator of polarization to an M1 macrophage is a transcription factor selected from IRF7 or a derivative thereof, or p65/RelA or a derivative thereof.
   Embodiment 525: A heterologous construct for stabilizing a macrophage in an M2 polarization state, comprising:
a) either (i) the regulatory element derived from a promoter of a gene that is more highly expressed in M2 macrophage compared to M1 or M0 macrophages, as applied to any one of embodiments 19-39, or (ii) the engineered macrophage-specific promoter of any one of embodiments 283-289, 293-301, 410-472, and 548-567; and
b) a heterologous payload encoding a master regulator of polarization to an M2 macrophage, wherein the regulatory element or engineered macrophage-specific promoter of (a) is operably linked to the heterologous payload and configured to induce expression of the heterologous payload.
   Embodiment 526: The heterologous construct of embodiment 525, wherein the master regulator of polarization to an M2 macrophage is IL-10, IL-4, IL-13, IL-21, TGF-beta, M-CSF, or a derivative thereof.
   Embodiment 527: The heterologous construct of embodiment 525 or 526, wherein the M2 state is an M2c state, an M2a state, or an M2b state.
   Embodiment 528: A vector comprising the heterologous construct according to any one of embodiments 473-527, 587-600.
   Embodiment 529: A dual expression vector comprising the heterologous construct according to any one of embodiments 473-527 or 587-600 and a second construct comprising a nucleotide sequence encoding an activating immune receptor.
   Embodiment 530: An immunoresponsive cell comprising the heterologous construct according to any one of embodiments 473-527 or 587-600, the vector according to embodiment 528, or the dual expression vector according to embodiment 529.
   Embodiment 531: The immunoresponsive cell according to embodiment 530, wherein the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.
   Embodiment 532: The immunoresponsive cell according to embodiment 531, wherein the immunoresponsive cell is a macrophage.
   Embodiment 533: The immunoresponsive cell of embodiment 532, wherein the macrophage is a tumor-resident macrophage.
   Embodiment 534: The immunoresponsive cell according to any one of embodiments 530-533, wherein the immunoresponsive cell expresses an activating immune receptor.
   Embodiment 535: The immunoresponsive cell according to embodiment 534, wherein the activating immune receptor comprises an antigen recognizing receptor.
   Embodiment 536: The immunoresponsive cell according to any one of embodiments 530-535, wherein the immunoresponsive cell is autologous.
   Embodiment 537: The immunoresponsive cell according to any one of embodiments 530-535, wherein the immunoresponsive cell is allogeneic.
   Embodiment 538: A pharmaceutical composition comprising the vector of embodiment 528, the dual expression vector according to embodiment 529, or the immunoresponsive cell according to any one of embodiments 530-537, and a pharmaceutically acceptable carrier, pharmaceutically acceptable excipient, or a combination thereof.
   Embodiment 539: A method of increasing expression of a target gene, the method comprising use of the engineered macrophage-specific promoter of any one of embodiments 1-472 and 548-586, the vector of embodiment 528, or the dual expression vector according to embodiment 529 to increase expression of the target gene.
   Embodiment 540: The method of embodiment 539, wherein the target gene is an immunomodulatory gene.
   Embodiment 541: A method of treating a subject in need thereof, the method comprising administering a therapeutically effective dose of the vector of embodiment 528, the dual expression vector according to embodiment 529, the immunoresponsive cell according to any one of embodiments 530-537, or the pharmaceutical composition according to embodiment 538.
   Embodiment 542: A kit for treating and/or preventing a disease or disorder, comprising the immunoresponsive cell according to any one of embodiments 530-537.
   Embodiment 543: A kit for treating and/or preventing a tumor, comprising the immunoresponsive cell according to any one of embodiments 530-537.
   Embodiment 544: The kit according to embodiment 542 or 543, wherein the kit further comprises written instructions for using the immunoresponsive cell for treating and/or preventing a tumor in a subject.
   Embodiment 545: A kit for treating and/or preventing a tumor, comprising the pharmaceutical composition according to embodiment 538.
   Embodiment 546: A kit for treating and/or preventing a disease or disorder, comprising the pharmaceutical composition according to embodiment 538.
   Embodiment 547: The kit according to embodiment 546, wherein the kit further comprises written instructions for using the pharmaceutical composition for treating and/or preventing a tumor in a subject.
   Embodiment 548: The engineered macrophage-specific promoter of embodiment 296, wherein:
i. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 420, and the minimal promoter comprises a sequence of a promoter selected from: minTK promoter; an SCP3 promoter, and a hybrid YBTATA-SCP3 (“YB-SCP3”) promoter;
ii. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 425, and the minimal promoter comprises a sequence of a promoter selected from: a minTK promoter, an SCP3 promoter, a YB-SCP3 promoter, a YBTATA promoter, and a minCMV promoter;
iii. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 426, and the minimal promoter comprises a sequence of a YB-SCP3 promoter;
iv. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 427, and the minimal promoter comprises a sequence of a minCMV promoter; or
v. the at least one regulatory element comprises a nucleotide sequence having at least 75%, at least 80%, at least 85%, at least 90%, at least 95%, at least 96%, at least 97%, at least 98%, at least 99% or 100% sequence identity to SEQ ID NO: 423, and the minimal promoter comprises a sequence of a minTK promoter.
   Embodiment 549: The engineered macrophage-specific promoter of embodiment 548, wherein the at least one regulatory element comprises a nucleotide sequence having at least 95% sequence identity to SEQ ID NO: 420.
   Embodiment 550: The engineered macrophage-specific promoter of embodiment 548, wherein the at least one regulatory element comprises a nucleotide sequence as set forth in SEQ ID NO: 420.
   Embodiment 551: The engineered macrophage-specific promoter of embodiment 548, wherein the at least one regulatory element comprises a nucleotide sequence having at least 95% sequence identity to SEQ ID NO: 425.
   Embodiment 552: The engineered macrophage-specific promoter of embodiment 548, wherein the at least one regulatory element comprises a nucleotide sequence as set forth in SEQ ID NO: 425.
   Embodiment 553: The engineered macrophage-specific promoter of embodiment 548, wherein the at least one regulatory element comprises a nucleotide sequence having at least 95% sequence identity to SEQ ID NO: 426.
   Embodiment 554: The engineered macrophage-specific promoter of embodiment 548, wherein the at least one regulatory element comprises a nucleotide sequence as set forth in SEQ ID NO: 426.
   Embodiment 555: The engineered macrophage-specific promoter of embodiment 548, wherein the at least one regulatory element comprises a nucleotide sequence having at least 95% sequence identity to SEQ ID NO: 427.
   Embodiment 556: The engineered macrophage-specific promoter of embodiment 548, wherein the at least one regulatory element comprises a nucleotide sequence as set forth in SEQ ID NO: 427.
   Embodiment 557: The engineered macrophage-specific promoter of embodiment 548, wherein the at least one regulatory element comprises a nucleotide sequence having at least 95% sequence identity to SEQ ID NO: 423.
   Embodiment 558: The engineered macrophage-specific promoter of embodiment 548, wherein the at least one regulatory element comprises a nucleotide sequence as set forth in SEQ ID NO: 423.
   Embodiment 559: The engineered macrophage-specific promoter of any one of embodiments 548-552, 556, or 557, wherein the minTK comprises a nucleotide sequence having at least 80% identity to SEQ ID NO: 448.
   Embodiment 560: The engineered macrophage-specific promoter of any one of embodiments 548-552, 556, or 557, wherein the minTK comprises a nucleotide sequence as set forth in SEQ ID NO: 448.
   Embodiment 561: The engineered macrophage-specific promoter of any one of embodiments 548-552, wherein the SCP3 comprises a nucleotide sequence having at least 80% identity to SEQ ID NO: 449.
   Embodiment 562: The engineered macrophage-specific promoter of any one of embodiments 548-552, wherein the SCP3 comprises a nucleotide sequence as set forth in SEQ ID NO: 449.
   Embodiment 563: The engineered macrophage-specific promoter of any one of embodiments 548-554, wherein the YB-SCP3 comprises a nucleotide sequence having at least 80% identity to SEQ ID NO: 450.
   Embodiment 564: The engineered macrophage-specific promoter of any one of embodiments 548-554, wherein the YB-SCP3 comprises a nucleotide sequence as set forth in SEQ ID NO: 450.
   Embodiment 565: The engineered macrophage-specific promoter of any one of embodiments 548, 551, 552, or 555-556, wherein the minCMV comprises a nucleotide sequence having at least 80% identity to SEQ ID NO: 447.
   Embodiment 566: The engineered macrophage-specific promoter of any one of embodiments 548, 551, 552, or 555-556, wherein the minCMV comprises a nucleotide sequence as set forth in SEQ ID NO: 447.
   Embodiment 567: The engineered macrophage-specific promoter of any one of embodiments 548-566, wherein the minimal promoter further comprises a flanking sequence.
   Embodiment 568: The engineered macrophage-specific promoter system of embodiment 12 or the engineered macrophage-specific promoter of embodiment 161, wherein the regulatory element or the engineered macrophage-specific promoter:
i. comprises a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256; and
ii. does not comprise at least one repressive element selected from: SEQ ID NO: 226, SEQ ID NO: 234, SEQ ID NO: 236, SEQ ID NO: 238, SEQ ID NO: 246, and SEQ ID NO: 252.
   Embodiment 569: The engineered macrophage-specific promoter system or the engineered macrophage-specific promoter of embodiment 568, further comprising a sixth transcriptional activating element as set forth in SEQ ID NO: 224 and/or a seventh transcriptional activating element as set forth in SEQ ID NO: 258.
   Embodiment 570: The engineered macrophage-specific promoter system or the engineered macrophage-specific promoter of any one of embodiments 568-569, wherein the regulatory element or the engineered macrophage-specific promoter further do not comprise SEQ ID NO: 228, SEQ ID NO: 230, SEQ ID NO: 232, SEQ ID NO: 242, SEQ ID NO:244, SEQ ID NO: 248, and SEQ ID NO: 250.
   Embodiment 571: The engineered macrophage-specific promoter system or the engineered macrophage-specific promoter of any one of embodiments 568-570, wherein the regulatory element or the engineered macrophage-specific promoter does not comprise the repressive elements as set forth in SEQ ID NO: 226, SEQ ID NO: 236, SEQ ID NO: 238, SEQ ID NO: 246, and SEQ ID NO: 252.
   Embodiment 572: The engineered macrophage-specific promoter system or the engineered macrophage-specific promoter of any one of embodiments 568-571, wherein the regulatory element or the engineered macrophage-specific promoter comprises a sequence as set forth in GTTAAGTGGCTAGGGATAACATTGAGGCACTAAAGCATTATTGGTTCTGCAGTCA AGGGTAGGATAGATTGTTTTTTTTTTTTT (SEQ ID NO: 482), and a sequence as set forth in

(SEQ ID NO: 483)
TTTGTGGTTTTATTGGTTTTCATATTACAAACAAAGAAACTAGAAAATGA
AACCATTCCAAAAGTGGAAGTAATTTCTCA

Embodiment 573: The engineered macrophage-specific promoter system or the engineered macrophage-specific promoter of any one of embodiments 568-572, wherein the regulatory element or the engineered macrophage-specific promoter comprises a sequence as set forth in GCTCTTCTAAAAATATGCGAAATGAGGTTTTTAGGGAGGTGTAGGTATGGCTGAA GAAAATCAAGGTGAATGAAGACAAGATCAATTGAGAATGTAGTTTCAGAAATAG CAAAGAAGCCAAAGTTTGAGGAAGTTAAGTGGCTAGGGATAACATTGAGGCACT AAAGCATTATTGGTTCTGCAGTCAAGGGTAGGATAGATTGTTTTTTTTTTTTTTGA GACGGAGTCTCACTCTGCTGCCCAGGC (SEQ ID NO: 484), a sequence as set forth in ATTTTGGTTTCAGTTTTCCTTAC (SEQ ID NO: 240), and a sequence as set forth in TTTGTGGTTTTATTGGTTTTCATATTACAAACAAAGAAACTAGAAAATGAAACCA TTCCAAAAGTGGAAGTAATTTCTCA (SEQ ID NO: 483).
Embodiment 574: The engineered macrophage-specific promoter system or the engineered macrophage-specific promoter of embodiment 568-573, wherein the regulatory element or the engineered macrophage-specific promoter comprises a nucleotide sequence at least 80%, 85%, 90%, 95%, 97.5%, 98%, 99%, or 100% identical to SEQ ID NO: 456.
Embodiment 575: The engineered macrophage-specific promoter system or the engineered macrophage-specific promoter of any one of embodiments 568, 569, or 571-573, wherein the regulatory element or the engineered macrophage-specific promoter comprises a nucleotide sequence at least 80%, 85%, 90%, 95%, 97.5%, 98%, 99%, or 100% identical to SEQ ID NO: 457.
Embodiment 576: The engineered macrophage-specific promoter system or the engineered macrophage-specific promoter of any one of embodiments 568, 569, or 571-573, wherein the regulatory element or the engineered macrophage-specific promoter comprises a nucleotide sequence at least 80%, 85%, 90%, 95%, 97.5%, 98%, 99%, or 100% identical to SEQ ID NO: 458.
Embodiment 577: The engineered macrophage-specific promoter system of embodiment 14 or the engineered macrophage-specific promoter of embodiment 224, wherein the regulatory element or the engineered macrophage-specific promoter:
i. comprises a first transcriptional activating element as set forth in SEQ ID NO: 268 and a second transcriptional activating element as set forth in SEQ ID NO: 270; and
ii. does not comprise at least one repressive element selected from: SEQ ID NO: 260, SEQ ID NO: 262, SEQ ID NO: 264, SEQ ID NO: 266, SEQ ID NO: 272, and SEQ ID NO: 391.
   Embodiment 578: The engineered macrophage-specific promoter system or the engineered macrophage-specific promoter of embodiment 577, wherein the regulatory element or the engineered macrophage-specific promoter comprises at least one, at least two, at least three, at least four, or at least five tandem repeats of SEQ ID NO: 268 and SEQ ID NO: 270.
   Embodiment 579: The engineered macrophage-specific promoter system or the engineered macrophage-specific promoter of any one of embodiments 577-578, further comprising a third transcriptional activating element as set forth in SEQ ID NO: 291 and/or a fourth transcriptional activating element as set forth in: SEQ ID NO: 295.
   Embodiment 580: The engineered macrophage-specific promoter system or the engineered macrophage-specific promoter of any one of embodiments 577-579, wherein the regulatory element or the engineered macrophage-specific promoter does not comprise the repressive elements as set forth in SEQ ID NO: 262, SEQ ID NO: 264, SEQ ID NO: 272, and SEQ ID NO: 391, optionally wherein the regulatory element or the engineered macrophage-specific promoter further does not comprise SEQ ID NO: 260 and/or SEQ ID NO: 266.
   Embodiment 581: The engineered macrophage-specific promoter system or the engineered macrophage-specific promoter of embodiment 577 or 580, wherein the regulatory element or the engineered macrophage-specific promoter comprise a nucleotide sequence at least 80%, 85%, 90%, 95%, 97.5%, 98%, 99%, or 100% identical SEQ ID NO: 459.
   Embodiment 582: The engineered macrophage-specific promoter system or the engineered macrophage-specific promoter of any one of embodiments 577, 578, or 580, wherein the regulatory element or the engineered macrophage-specific promoter comprise the nucleotide sequence as set forth in SEQ ID NO: 460.
   Embodiment 583: The engineered macrophage-specific promoter system or the engineered macrophage-specific promoter of any one of embodiments 577, 579, or 580, wherein the regulatory element or the engineered macrophage-specific promoter comprise the nucleotide sequence as set forth in SEQ ID NO: 461.
   Embodiment 584: The engineered macrophage-specific promoter system or the engineered macrophage-specific promoter of any one of embodiments 577-583, wherein the regulatory element or the engineered macrophage-specific promoter is operably linked to a minimal promoter, wherein optionally the minimal promoter comprises a sequence of a promoter selected from minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, SCP3, YB-SCP3, inducer molecule responsive promoters, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, hUBIb, and tandem repeats thereof.
   Embodiment 585: The engineered macrophage-specific promoter system or the engineered macrophage-specific promoter of embodiment 584, wherein the minimal promoter comprises a YB TATA promoter sequence.
   Embodiment 586: The engineered macrophage-specific promoter system or the engineered macrophage-specific promoter of any one of embodiments 577-585, further comprising a translation initiator site, optionally wherein the translation initiator site is or comprises a Kozak sequence.
   Embodiment 587: The heterologous construct of embodiment 486 or embodiment 489, wherein the cytokine is modified to comprise a membrane tethering domain.
   Embodiment 588: The heterologous construct of embodiment 587, wherein the membrane tethering domain is or comprises a transmembrane-intracellular domain and/or transmembrane domain of a protein selected from: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, and BTLA, or a functional portion thereof.
   Embodiment 589: The heterologous construct of embodiment 587, wherein the membrane tethering domain is or comprises a transmembrane domain of B7-1 protein, or a functional portion thereof.
   Embodiment 590: The heterologous construct of any one of embodiments 587-589, wherein the cytokine is IFNgamma.
   Embodiment 591: The heterologous construct of any one of embodiments 587-590, wherein the cytokine and the tethering domain are linked by a linker.
   Embodiment 592: The heterologous construct of embodiment 524, wherein the master regulator of polarization to an M1 macrophage is IRF7 or a derivative thereof.
   Embodiment 593: The heterologous construct of embodiment 592, wherein the derivative of IRF7 comprises IRF7 operably linked to a degron domain.
   Embodiment 594: The heterologous construct of embodiment 593, wherein the degron domain is selected from: a PEST domain, HCV NS4 degron, GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 190) phospho-dependent degron, an Siah binding motif, an SPOP SBC docking motif, a PCNA binding PIP box, and derivatives thereof.
   Embodiment 595: The heterologous construct of embodiment 594, wherein the degron domain is a PEST domain, optionally wherein the PEST comprises the amino acid sequence SEQ ID NO: 501 or a derivative thereof.
   Embodiment 596: The heterologous construct of any one of embodiments 592-595, wherein the engineered macrophage specific promoter comprises a regulatory element selected from:
i. a nucleotide sequence having at least 75%, at least 80%, at least 85%, at least 90%, at least 95%, at least 96%, at least 97%, at least 98%, at least 99% or 100% sequence identity to SEQ ID NO: 420; and ii. a nucleotide sequence having at least 75%, at least 80%, at least 85%, at least 90%, at least 95%, at least 96%, at least 97%, at least 98%, at least 99% or 100% sequence identity to SEQ ID NO: 427.
   Embodiment 597: The heterologous construct of embodiment 596, wherein the engineered macrophage specific promoter comprises a regulatory element having at least 95% sequence identity to SEQ ID NO: 420, optionally having 100% sequence identity to SEQ ID NO: 420.
   Embodiment 598: The heterologous construct of embodiment 597, wherein the regulatory element is operably linked to a minTK minimal promoter or SCP3 minimal promoter.
   Embodiment 599: The heterologous construct of embodiment 596, wherein the engineered macrophage specific promoter comprises a regulatory element having at least 95% sequence identity to SEQ ID NO: 427, optionally having 100% sequence identity to SEQ ID NO: 427.
   Embodiment 600: The heterologous construct of embodiment 599, wherein the regulatory element is operably linked to a minCMV promoter.

### Additional Sequence Tables

TABLE 7
Sequences Of Exemplary Effector Molecules And
Reporter Molecules

| Name | |
|---|---|
| | IFN-gamma |
| Nucleotide | ATGAAATATACAAGTTATATCTTGGCTTTTCAGCTCTGCATCGTTTTGGGTT |
| | CTCTTGGCTGTTACTGCCAGGACCCATATGTAAAAGAAGCAGAAAACCTTAA |
| | GAAATATTTTAATGCAGGTCATTCAGATGTAGCGGATAATGGAACTCTTTTC |
| | TTAGGCATTTTGAAGAATTGGAAAGAGGAGAGTGACAGAAAAATAATGCAGA |
| | GCCAAATTGTCTCCTTTTACTTCAAACTTTTTAAAAACTTTAAAGATGACCA |
| | GAGCATCCAAAAGAGTGTGGAGACCATCAAGGAAGACATGAATGTCAAGTTT |
| | TTCAATAGCAACAAAAAGAAACGAGATGACTTCGAAAAGCTGACTAATTATT |
| | CGGTAACTGACTTGAATGTCCAACGCAAAGCAATACATGAACTCATCCAAGT |
| | GATGGCTGAACTGTCGCCAGCAGCTAAAACAGGGAAGCGAAAAAGGAGTCAG |
| | ATGCTGTTTCGAGGTCGAAGAGCATCCCAG (SEQ ID NO: 394) |
| Amino acid | MKYTSYILAFQLCIVLGSLGCYCQDPYVKEAENLKKYFNAGHSDVADNGTLF |
| | LGILKNWKEESDRKIMQSQIVSFYFKLFKNFKDDQSIQKSVETIKEDMNVKF |
| | FNSNKKKRDDFEKLTNYSVTDLNVQRKAIHELIQVMAELSPAAKTGKRKRSQ |
| | MLFRGRRASQ (SEQ ID NO: 395) |
| | TNF-alpha |
| Nucleotide | ATGAGCACTGAAAGCATGATCCGGGACGTGGAGCTGGCCGAGGAGGCGCTCC |
| | CCAAGAAGACAGGGGGGCCCCAGGGCTCCAGGCGGTGCTTGTTCCTCAGCCT |
| | CTTCTCCTTCCTGATCGTGGCAGGCGCCACCACGCTCTTCTGCCTGCTGCAC |
| | TTTGGAGTGATCGGCCCCCAGAGGGAAGAGTTCCCCAGGGACCTCTCTCTAA |
| | TCAGCCCTCTGGCCCAGGCAGTCAGATCATCTTCTCGAACCCCGAGTGACAA |
| | GCCTGTAGCCCATGTTGTAGCAAACCCTCAAGCTGAGGGGCAGCTCCAGTGG |
| | CTGAACCGCCGGGCCAATGCCCTCCTGGCCAATGGCGTGGAGCTGAGAGATA |
| | ACCAGCTGGTGGTGCCATCAGAGGGCCTGTACCTCATCTACTCCCAGGTCCT |
| | CTTCAAGGGCCAAGGCTGCCCCTCCACCCATGTGCTCCTCACCCACACCATC |
| | AGCCGCATCGCCGTCTCCTACCAGACCAAGGTCAACCTCCTCTCTGCCATCA |
| | AGAGCCCCTGCCAGAGGGAGACCCCAGAGGGGGCTGAGGCCAAGCCCTGGTA |
| | TGAGCCCATCTATCTGGGAGGGGTCTTCCAGCTGGAGAAGGGTGACCGACTC |
| | AGCGCTGAGATCAATCGGCCCGACTATCTCGACTTTGCCGAGTCTGGGCAGG |
| | TCTACTTTGGGATCATTGCCCTG (SEQ ID NO: 396) |
| Amino acid | MSTESMIRDVELAEEALPKKTGGPQGSRRCLFLSLFSFLIVAGATTLFCLLH |
| | FGVIGPQREEFPRDLSLISPLAQAVRSSSRTPSDKPVAHVVANPQAEGQLQW |
| | LNRRANALLANGVELRDNQLVVPSEGLYLIYSQVLFKGQGCPSTHVLLTHTI |
| | SRIAVSYQTKVNLLSAIKSPCQRETPEGAEAKPWYEPIYLGGVFQLEKGDRL |
| | SAEINRPDYLDFAESGQVYFGIIAL (SEQ ID NO: 397) |
| | IL-12p70 |
| Nucleotide | ATGTGCCATCAGCAACTCGTCATCTCCTGGTTCTCCCTTGTGTTCCTCGCTT |
| | CCCCTCTGGTCGCCATTTGGGAACTGAAGAAGGACGTCTACGTGGTCGAGCT |
| | GGATTGGTACCCGGACGCCCCTGGAGAAATGGTCGTGCTGACTTGCGATACG |
| | CCAGAAGAGGACGGCATAACCTGGACCCTGGATCAGAGCTCCGAGGTGCTCG |
| | GAAGCGGAAAGACCCTGACCATTCAAGTCAAGGAGTTCGGCGACGCGGGCCA |
| | GTACACTTGCCACAAGGGTGGCGAAGTGCTGTCCCACTCCCTGCTGCTGCTG |
| | CACAAGAAAGAGGATGGAATCTGGTCCACTGACATCCTCAAGGACCAAAAAG |
| | AACCGAAGAACAAGACCTTCCTCCGCTGCGAAGCCAAGAACTACAGCGGTCG |
| | GTTCACCTGTTGGTGGCTGACGACAATCTCCACCGACCTGACTTTCTCCGTG |
| | AAGTCGTCACGGGGATCAAGCGATCCTCAGGGCGTGACCTGTGGAGCCGCCA |
| | CTCTGTCCGCCGAGAGAGTCAGGGGAGACAACAAGGAATATGAGTACTCCGT |
| | GGAATGCCAGGAGGACAGCGCCTGCCCTGCCGCGGAAGAGTCCCTGCCTATC |
| | GAGGTCATGGTCGATGCCGTGCATAAGCTGAAATACGAGAACTACACTTCCT |
| | CCTTCTTTATCCGCGACATCATCAAGCCTGACCCCCCCAAGAACTTGCAGCT |
| | GAAGCCACTCAAGAACTCCCGCCAAGTGGAAGTGTCTTGGGAATATCCAGAC |
| | ACTTGGAGCACCCCGCACTCATACTTCTCGCTCACTTTCTGTGTGCAAGTGC |
| | AGGGAAAGTCCAAACGGGAGAAGAAAGACCGGGTGTTCACCGACAAAACCTC |
| | CGCCACTGTGATTTGTCGGAAGAACGCGTCAATCAGCGTCCGGGCGCAGGAT |
| | AGATACTACTCGTCCTCCTGGAGCGAATGGGCCAGCGTGCCTTGTTCCGGTG |
| | GCGGATCAGGCGGAGGTTCAGGAGGAGGCTCCGGAGGAGGTTCCCGGAACCT |
| | CCCTGTGGCAACCCCCGACCCTGGAATGTTCCCGTGCCTACACCACTCCCAA |
| | AACCTCCTGAGGGCTGTGTCGAACATGTTGCAGAAGGCCCGCCAGACCCTTG |
| | AGTTCTACCCCTGCACCTCGGAAGAAATTGATCACGAGGACATCACCAAGGA |
| | CAAGACCTCGACCGTGGAAGCCTGCCTGCCGCTGGAACTGACCAAGAACGAA |
| | TCGTGTCTGAACTCCCGCGAGACAAGCTTTATCACTAACGGCAGCTGCCTGG |
| | CGTCGAGAAAGACCTCATTCATGATGGCGCTCTGTCTTTCCTCGATCTACGA |
| | AGATCTGAAGATGTATCAGGTCGAGTTCAAGACCATGAACGCCAAGCTGCTC |
| | ATGGACCCGAAGCGGCAGATCTTCCTGGACCAGAATATGCTCGCCGTGATTG |
| | ATGAACTGATGCAGGCCCTGAATTTCAACTCCGAGACTGTGCCTCAAAAGTC |
| | CAGCCTGGAAGAACCGGACTTCTACAAGACCAAGATCAAGCTGTGCATCCTG |
| | TTGCACGCTTTCCGCATTCGAGCCGTGACCATTGACCGCGTGATGTCCTACC |
| | TGAACGCCAGT (SEQ ID NO: 398) |
| Amino acid | MCHQQLVISWFSLVFLASPLVAIWELKKDVYVVELDWYPDAPGEMVVLTCDT |
| | PEEDGITWTLDQSSEVLGSGKTLTIQVKEFGDAGQYTCHKGGEVLSHSLLLL |
| | HKKEDGIWSTDILKDQKEPKNKTFLRCEAKNYSGRFTCWWLTTISTDLTFSV |
| | KSSRGSSDPQGVTCGAATLSAERVRGDNKEYEYSVECQEDSACPAAEESLPI |
| | EVMVDAVHKLKYENYTSSFFIRDIIKPDPPKNLQLKPLKNSRQVEVSWEYPD |
| | TWSTPHSYFSLTFCVQVQGKSKREKKDRVFTDKTSATVICRKNASISVRAQD |
| | RYYSSSWSEWASVPCSGGGSGGGSGGGSGGGSRNLPVATPDPGMFPCLHHSQ |
| | NLLRAVSNMLQKARQTLEFYPCTSEEIDHEDITKDKTSTVEACLPLELTKNE |
| | SCLNSRETSFITNGSCLASRKTSFMMALCLSSIYEDLKMYQVEFKTMNAKLL |
| | MDPKRQIFLDQNMLAVIDELMQALNFNSETVPQKSSLEEPDFYKTKIKLCIL |
| | LHAFRIRAVTIDRVMSYLNAS (SEQ ID NO: 399) |
| | IRF7 |
| Nucleotide | ATGGCCTTGGCTCCTGAGAGGGCAGCCCCACGCGTGCTGTTCGGAGAGTGGC |
| | TCCTTGGAGAGATCAGCAGCGGCTGCTATGAGGGGCTGCAGTGGCTGGACGA |
| | GGCCCGCACCTGTTTCCGCGTGCCCTGGAAGCACTTCGCGCGCAAGGACCTG |
| | AGCGAGGCCGACGCGCGCATCTTCAAGGCCTGGGCTGTGGCCCGCGGCAGGT |
| | GGCCGCCTAGCAGCAGGGGAGGTGGCCCGCCCCCCGAGGCTGAGACTGCGGA |
| | GCGCGCCGGCTGGAAAACCAACTTCCGCTGCGCACTGCGCAGCACGCGTCGC |
| | TTCGTGATGCTGCGGGATAACTCGGGGGACCCGGCCGACCCGCACAAGGTGT |
| | ACGCGCTCAGCCGGGAGCTGTGCTGGCGAGAAGGCCCAGGCACGGACCAGAC |
| | TGAGGCAGAGGCCCCCGCAGCTGTCCCACCACCACAGGGTGGGCCCCCAGGG |
| | CCATTCCTGGCACACACACATGCTGGACTCCAAGCCCCAGGCCCCCTCCCTG |
| | CCCCAGCTGGTGACAAGGGGGACCTCCTGCTCCAGGCAGTGCAACAGAGCTG |
| | CCTGGCAGACCATCTGCTGACAGCGTCATGGGGGGCAGATCCAGTCCCAACC |
| | AAGGCTCCTGGAGAGGGACAAGAAGGGCTTCCCCTGACTGGGGCCTGTGCTG |
| | GAGGCCCAGGGCTCCCTGCTGGGGAGCTGTACGGGTGGGCAGTAGAGACGAC |
| | CCCCAGCCCCGGGCCCCAGCCCGCGGCACTAACGACAGGCGAGGCCGCGGCC |
| | CCAGAGTCCCCGCACCAGGCAGAGCCGTACCTGTCACCCTCCCCAAGCGCCT |
| | GCACCGCGGTGCAAGAGCCCAGCCCAGGGGCGCTGGACGTGACCATCATGTA |
| | CAAGGGCCGCACGGTGCTGCAGAAGGTGGTGGGACACCCGAGCTGCACGTTC |
| | CTATACGGCCCCCCAGACCCAGCTGTCCGGGCCACAGACCCCCAGCAGGTAG |
| | CATTCCCCAGCCCTGCCGAGCTCCCGGACCAGAAGCAGCTGCGCTACACGGA |
| | GGAACTGCTGCGGCACGTGGCCCCTGGGTTGCACCTGGAGCTTCGGGGGCCA |
| | CAGCTGTGGGCCCGGCGCATGGGCAAGTGCAAGGTGTACTGGGAGGTGGGCG |
| | GACCCCCAGGCTCCGCCAGCCCCTCCACCCCAGCCTGCCTGCTGCCTCGGAA |
| | CTGTGACACCCCCATCTTCGACTTCAGAGTCTTCTTCCAAGAGCTGGTGGAA |
| | TTCCGGGCACGGCAGCGCCGTGGCTCCCCACGCTATACCATCTACCTGGGCT |
| | TCGGGCAGGACCTGTCAGCTGGGAGGCCCAAGGAGAAGAGCCTGGTCCTGGT |
| | GAAGCTGGAACCCTGGCTGTGCCGAGTGCACCTAGAGGGCACGCAGCGTGAG |
| | GGTGTGTCTTCCCTGGATAGCAGCAGCCTCAGCCTCTGCCTGTCCAGCGCCA |
| | ACAGCCTCTATGACGACATCGAGTGCTTCCTTATGGAGCTGGAGCAGCCCGC |
| | C (SEQ ID NO: 400) |
| Amino acid | MALAPERAAPRVLFGEWLLGEISSGCYEGLQWLDEARTCFRVPWKHFARKDL |
| | SEADARIFKAWAVARGRWPPSSRGGGPPPEAETAERAGWKTNFRCALRSTRR |
| | FVMLRDNSGDPADPHKVYALSRELCWREGPGTDQTEAEAPAAVPPPQGGPPG |
| | PFLAHTHAGLQAPGPLPAPAGDKGDLLLQAVQQSCLADHLLTASWGADPVPT |
| | KAPGEGQEGLPLTGACAGGPGLPAGELYGWAVETTPSPGPQPAALTTGEAAA |
| | PESPHQAEPYLSPSPSACTAVQEPSPGALDVTIMYKGRTVLQKVVGHPSCTF |
| | LYGPPDPAVRATDPQQVAFPSPAELPDQKQLRYTEELLRHVAPGLHLELRGP |
| | QLWARRMGKCKVYWEVGGPPGSASPSTPACLLPRNCDTPIFDFRVFFQELVE |
| | FRARQRRGSPRYTIYLGFGQDLSAGRPKEKSLVLVKLEPWLCRVHLEGTQRE |
| | GVSSLDSSSLSLCLSSANSLYDDIECFLMELEQPA (SEQ ID NO: 401) |
| | p65/RelA |
| Nucleotide | ATGGACGAACTGTTCCCCCTCATCTTCCCGGCAGAGCCAGCCCAGGCCTCTG |
| | GCCCCTATGTGGAGATCATTGAGCAGCCCAAGCAGCGGGGCATGCGCTTCCG |
| | CTACAAGTGCGAGGGGCGCTCCGCGGGCAGCATCCCAGGCGAGAGGAGCACA |
| | GATACCACCAAGACCCACCCCACCATCAAGATCAATGGCTACACAGGACCAG |
| | GGACAGTGCGCATCTCCCTGGTCACCAAGGACCCTCCTCACCGGCCTCACCC |
| | CCACGAGCTTGTAGGAAAGGACTGCCGGGATGGCTTCTATGAGGCTGAGCTC |
| | TGCCCGGACCGCTGCATCCACAGTTTCCAGAACCTGGGAATCCAGTGTGTGA |
| | AGAAGCGGGACCTGGAGCAGGCTATCAGTCAGCGCATCCAGACCAACAACAA |
| | CCCCTTCCAAGTTCCTATAGAAGAGCAGCGTGGGGACTACGACCTGAATGCT |
| | GTGCGGCTCTGCTTCCAGGTGACAGTGCGGGACCCATCAGGCAGGCCCCTCC |
| | GCCTGCCGCCTGTCCTTTCTCATCCCATCTTTGACAATCGTGCCCCCAACAC |
| | TGCCGAGCTCAAGATCTGCCGAGTGAACCGAAACTCTGGCAGCTGCCTCGGT |
| | GGGGATGAGATCTTCCTACTGTGTGACAAGGTGCAGAAAGAGGACATTGAGG |
| | TGTATTTCACGGGACCAGGCTGGGAGGCCCGAGGCTCCTTTTCGCAAGCTGA |
| | TGTGCACCGACAAGTGGCCATTGTGTTCCGGACCCCTCCCTACGCAGACCCC |
| | AGCCTGCAGGCTCCTGTGCGTGTCTCCATGCAGCTGCGGCGGCCTTCCGACC |
| | GGGAGCTCAGTGAGCCCATGGAATTCCAGTACCTGCCAGATACAGACGATCG |
| | TCACCGGATTGAGGAGAAACGTAAAAGGACATATGAGACCTTCAAGAGCATC |
| | ATGAAGAAGAGTCCTTTCAGCGGACCCACCGACCCCCGGCCTCCACCTCGAC |
| | GCATTGCTGTGCCTTCCCGCAGCTCAGCTTCTGTCCCCAAGCCAGCACCCCA |
| | GCCCTATCCCTTTACGTCATCCCTGAGCACCATCAACTATGATGAGTTTCCC |
| | ACCATGGTGTTTCCTTCTGGGCAGATCAGCCAGGCCTCGGCCTTGGCCCCGG |
| | CCCCTCCCCAAGTCCTGCCCCAGGCTCCAGCCCCTGCCCCTGCTCCAGCCAT |
| | GGTATCAGCTCTGGCCCAGGCCCCAGCCCCTGTCCCAGTCCTAGCCCCAGGC |
| | CCTCCTCAGGCTGTGGCCCCACCTGCCCCCAAGCCCACCCAGGCTGGGGAAG |
| | GAACGCTGTCAGAGGCCCTGCTGCAGCTGCAGTTTGATGATGAAGACCTGGG |
| | GGCCTTGCTTGGCAACAGCACAGACCCAGCTGTGTTCACAGACCTGGCATCC |
| | GTCGACAACTCCGAGTTTCAGCAGCTGCTGAACCAGGGCATACCTGTGGCCC |
| | CCCACACAACTGAGCCCATGCTGATGGAGTACCCTGAGGCTATAACTCGCCT |
| | AGTGACAGGGGCCCAGAGGCCCCCCGACCCAGCTCCTGCTCCACTGGGGGCC |
| | CCGGGGCTCCCCAATGGCCTCCTTTCAGGAGATGAAGACTTCTCCTCCATTG |
| | CGGACATGGACTTCTCAGCCCTGCTGAGTCAGATCAGCTCC (SEQ ID |
| | NO: 402) |
| Amino acid | MDELFPLIFPAEPAQASGPYVEIIEQPKQRGMRFRYKCEGRSAGSIPGERST |
| | DTTKTHPTIKINGYTGPGTVRISLVTKDPPHRPHPHELVGKDCRDGFYEAEL |
| | CPDRCIHSFQNLGIQCVKKRDLEQAISQRIQTNNNPFQVPIEEQRGDYDLNA |
| | VRLCFQVTVRDPSGRPLRLPPVLSHPIFDNRAPNTAELKICRVNRNSGSCLG |
| | GDEIFLLCDKVQKEDIEVYFTGPGWEARGSFSQADVHRQVAIVFRTPPYADP |
| | SLQAPVRVSMQLRRPSDRELSEPMEFQYLPDTDDRHRIEEKRKRTYETFKSI |
| | MKKSPFSGPTDPRPPPRRIAVPSRSSASVPKPAPQPYPFTSSLSTINYDEFP |
| | TMVFPSGQISQASALAPAPPQVLPQAPAPAPAPAMVSALAQAPAPVPVLAPG |
| | PPQAVAPPAPKPTQAGEGTLSEALLQLQFDDEDLGALLGNSTDPAVFTDLAS |
| | VDNSEFQQLLNQGIPVAPHTTEPMLMEYPEAITRLVTGAQRPPDPAPAPLGA |
| | PGLPNGLLSGDEDFSSIADMDFSALLSQISS (SEQ ID NO: 403) |
| | IL-10 |
| Nucleotide | ATGCACAGCTCAGCACTGCTCTGTTGCCTGGTCCTCCTGACTGGGGTGAGGG |
| | CCAGCCCAGGCCAGGGCACCCAGTCTGAGAACAGCTGCACCCACTTCCCAGG |
| | CAACCTGCCTAACATGCTTCGAGATCTCCGAGATGCCTTCAGCAGAGTGAAG |
| | ACTTTCTTTCAAATGAAGGATCAGCTGGACAACTTGTTGTTAAAGGAGTCCT |
| | TGCTGGAGGACTTTAAGGGTTACCTGGGTTGCCAAGCCTTGTCTGAGATGAT |
| | CCAGTTTTACCTGGAGGAGGTGATGCCCCAAGCTGAGAACCAAGACCCAGAC |
| | ATCAAGGCGCATGTGAACTCCCTGGGGGAGAACCTGAAGACCCTCAGGCTGA |
| | GGCTACGGCGCTGTCATCGATTTCTTCCCTGTGAAAACAAGAGCAAGGCCGT |
| | GGAGCAGGTGAAGAATGCCTTTAATAAGCTCCAAGAGAAAGGCATCTACAAA |
| | GCCATGAGTGAGTTTGACATCTTCATCAACTACATAGAAGCCTACATGACAA |
| | TGAAGATACGAAAC (SEQ ID NO: 404) |
| Amino acid | MHSSALLCCLVLLTGVRASPGQGTQSENSCTHFPGNLPNMLRDLRDAFSRVK |
| | TFFQMKDQLDNLLLKESLLEDFKGYLGCQALSEMIQFYLEEVMPQAENQDPD |
| | IKAHVNSLGENLKTLRLRLRRCHRFLPCENKSKAVEQVKNAFNKLQEKGIYK |
| | AMSEFDIFINYIEAYMTMKIRN (SEQ ID NO: 405) |
| | IL-4 |
| Nucleotide | ATGGGTCTCACCTCCCAACTGCTTCCCCCTCTGTTCTTCCTGCTAGCATGTG |
| | CCGGCAACTTTGTCCACGGACACAAGTGCGATATCACCTTACAGGAGATCAT |
| | CAAAACTTTGAACAGCCTCACAGAGCAGAAGACTCTGTGCACCGAGTTGACC |
| | GTAACAGACATCTTTGCTGCCTCCAAGAACACAACTGAGAAGGAAACCTTCT |
| | GCAGGGCTGCGACTGTGCTCCGGCAGTTCTACAGCCACCATGAGAAGGACAC |
| | TCGCTGCCTGGGTGCGACTGCACAGCAGTTCCACAGGCACAAGCAGCTGATC |
| | CGATTCCTGAAACGGCTCGACAGGAACCTCTGGGGCCTGGCGGGCTTGAATT |
| | CCTGTCCTGTGAAGGAAGCCAACCAGAGTACGTTGGAAAACTTCTTGGAAAG |
| | GCTAAAGACGATCATGAGAGAGAAATATTCAAAGTGTTCGAGC (SEQ ID |
| | NO: 406) |
| Amino acid | MGLTSQLLPPLFFLLACAGNFVHGHKCDITLQEIIKTLNSLTEQKTLCTELT |
| | VTDIFAASKNTTEKETFCRAATVLRQFYSHHEKDTRCLGATAQQFHRHKQLI |
| | RFLKRLDRNLWGLAGLNSCPVKEANQSTLENFLERLKTIMREKYSKCSS |
| | (SEQ ID NO: 407) |
| | EGFP |
| Nucleotide | ATGGTGAGCAAGGGCGAGGAGCTGTTCACCGGGGTGGTGCCCATCCTGGTCG |
| | AGCTGGACGGCGACGTAAACGGCCACAAGTTCAGCGTGTCCGGCGAGGGCGA |
| | GGGCGATGCCACCTACGGCAAGCTGACCCTGAAGTTCATCTGCACCACCGGC |
| | AAGCTGCCCGTGCCCTGGCCCACCCTCGTGACCACCCTGACCTACGGCGTGC |
| | AGTGCTTCAGCCGCTACCCCGACCACATGAAGCAGCACGACTTCTTCAAGTC |
| | CGCCATGCCCGAAGGCTACGTCCAGGAGCGCACCATCTTCTTCAAGGACGAC |
| | GGCAACTACAAGACCCGCGCCGAGGTGAAGTTCGAGGGCGACACCCTGGTGA |
| | ACCGCATCGAGCTGAAGGGCATCGACTTCAAGGAGGACGGCAACATCCTGGG |
| | GCACAAGCTGGAGTACAACTACAACAGCCACAACGTCTATATCATGGCCGAC |
| | AAGCAGAAGAATGGCATCAAGGTGAACTTCAAGATCCGCCACAACATCGAGG |
| | ACGGCAGCGTGCAGCTCGCCGACCACTACCAGCAGAACACCCCCATCGGCGA |
| | CGGCCCCGTGCTGCTGCCCGACAACCACTACCTGAGCACCCAGTCCGCCCTG |
| | AGCAAAGACCCCAACGAGAAGCGCGATCACATGGTCCTGCTGGAGTTCGTGA |
| | CCGCCGCCGGGATCACTCTCGGCATGGACGAGCTGTACAAG (SEQ ID |
| | NO: 408) |
| Amino acid | MVSKGEELFTGVVPILVELDGDVNGHKFSVSGEGEGDATYGKLTLKFICTTG |
| | KLPVPWPTLVTTLTYGVQCFSRYPDHMKQHDFFKSAMPEGYVQERTIFFKDD |
| | GNYKTRAEVKFEGDTLVNRIELKGIDFKEDGNILGHKLEYNYNSHNVYIMAD |
| | KQKNGIKVNFKIRHNIEDGSVQLADHYQQNTPIGDGPVLLPDNHYLSTQSAL |
| | SKDPNEKRDHMVLLEFVTAAGITLGMDELYK (SEQ ID NO: 409) |
| | mCherry |
| Nucleotide | atggtgagcaagggcgaggaggataacatggccatcatcaaggagttcatgc |
| | gcttcaaggtgcacatggagggctccgtgaacggccacgagttcgagatcga |
| | gggcgagggcgagggccgcccctacgagggcacccagaccgccaagctgaag |
| | gtgaccaagggtggccccctgcccttcgcctgggacatcctgtcccctcagt |
| | tcatgtacggctccaaggcctacgtgaagcaccccgccgacatccccgacta |
| | cttgaagctgtccttccccgagggcttcaagtgggagcgcgtgatgaacttc |
| | gaggacggcggcgtggtgaccgtgacccaggactcctccctgcaggacggcg |
| | agttcatctacaaggtgaagctgcgcggcaccaacttcccctccgacggccc |
| | cgtaatgcagaagaagaccatgggctggcaggcctcctccgagcggatgtac |
| | cccgaggacggcgccctgaagggcgagatcaagcagaggctgaagctgaagg |
| | acggcggccactacgacgctgaggtcaagaccacctacaaggccaagaagcc |
| | cgtgcagctgcccggcgcctacaacgtcaacatcaagttggacatcacctcc |
| | cacaacgaggactacaccatcgtggaacagtacgaacgcgccgagggccgcc |
| | actccaccggcggcatggacgagctgtacaag (SEQ ID NO: 410) |
| Amino acid | MVSKGEEDNMAIIKEFMRFKVHMEGSVNGHEFEIEGEGEGRPYEGTQTAKLK |
| | VTKGGPLPFAWDILSPQFMYGSKAYVKHPADIPDYLKLSFPEGFKWERVMNF |
| | EDGGVVTVTQDSSLQDGEFIYKVKLRGTNFPSDGPVMQKKTMGWQASSERMY |
| | PEDGALKGEIKQRLKLKDGGHYDAEVKTTYKAKKPVQLPGAYNVNIKLDITS |
| | HNEDYTIVEQYERAEGRHSTGGMDELYK (SEQ ID NO: 411) |
| | nanoLuc |
| Nucleotide | ATGAACAGCTTCAGCACCAGCGCCTTTGGACCCGTGGCCTTTTCTCTGGGAC |
| | TGCTGCTGGTTCTGCCTGCCGCTTTTCCTGCTCCTGTGTTCACCCTGGAAGA |
| | TTTCGTCGGCGATTGGAGACAGACCGCCGGCTACAATCTGGACCAGGTGTTG |
| | GAGCAAGGCGGCGTGTCCAGCCTGTTTCAGAACCTGGGAGTGTCCGTGACAC |
| | CCATCCAGAGAATCGTGCTGAGCGGCGAGAACGGCCTGAAGATCGACATCCA |
| | CGTGATCATCCCTTACGAGGGCCTGTCCGGCGATCAGATGGGACAGATCGAG |
| | AAGATCTTTAAGGTGGTGTACCCCGTGGACGACCACCACTTCAAAGTGATCC |
| | TGCACTACGGCACCCTGGTCATCGATGGCGTGACCCCTAACATGATCGACTA |
| | CTTCGGCAGACCCTACGAGGGAATCGCCGTGTTCGACGGCAAGAAAATCACC |
| | GTGACCGGCACACTGTGGAACGGCAACAAGATCATCGACGAGCGGCTGATCA |
| | ACCCCGATGGCAGCCTGCTGTTCAGAGTGACCATCAACGGCGTGACAGGATG |
| | GCGGCTGTGCGAGAGAATTCTGGCC (SEQ ID NO: 412) |
| Amino acid | MNSFSTSAFGPVAFSLGLLLVLPAAFPAPVFTLEDFVGDWRQTAGYNLDQVL |
| | EQGGVSSLFQNLGVSVTPIQRIVLSGENGLKIDIHVIIPYEGLSGDQMGQIE |
| | KIFKVVYPVDDHHFKVILHYGTLVIDGVTPNMIDYFGRPYEGIAVFDGKKIT |
| | VTGTLWNGNKIIDERLINPDGSLLFRVTINGVTGWRLCERILA (SEQ ID |
| | NO: 413) |
| | Tethered IL-10 |
| Nucleotide | ATGCACAGCTCAGCACTGCTCTGTTGCCTGGTCCTCCTGACTGGGGTGAGGG |
| | CCAGCCCAGGCCAGGGCACCCAGTCTGAGAACAGCTGCACCCACTTCCCAGG |
| | CAACCTGCCTAACATGCTTCGAGATCTCCGAGATGCCTTCAGCAGAGTGAAG |
| | ACTTTCTTTCAAATGAAGGATCAGCTGGACAACTTGTTGTTAAAGGAGTCCT |
| | TGCTGGAGGACTTTAAGGGTTACCTGGGTTGCCAAGCCTTGTCTGAGATGAT |
| | CCAGTTTTACCTGGAGGAGGTGATGCCCCAAGCTGAGAACCAAGACCCAGAC |
| | ATCAAGGCGCATGTGAACTCCCTGGGGGAGAACCTGAAGACCCTCAGGCTGA |
| | GGCTACGGCGCTGTCATCGATTTCTTCCCTGTGAAAACAAGAGCAAGGCCGT |
| | GGAGCAGGTGAAGAATGCCTTTAATAAGCTCCAAGAGAAAGGCATCTACAAA |
| | GCCATGAGTGAGTTTGACATCTTCATCAACTACATAGAAGCCTACATGACAA |
| | TGAAGATACGAAACTCAGGCGGCGGTGGTAGTGGAGGCGGAGGCTCAGGCGG |
| | CGGAGGTTCCGGAGGTGGCGGTTCCGGCGGAGGATCTCTTCAATTGCTGCCT |
| | AGCTGGGCCATCACACTGATCTCCGTGAACGGCATCTTCGTGATCTGCTGCC |
| | TGACCTACTGCTTCGCCCCTAGATGCAGAGAGCGGAGAAGAAACGAGCGGCT |
| | GAGAAGAGAAAGCGTGCGGCCTGTG (SEQ ID NO: 466) |
| Amino acid | MHSSALLCCLVLLTGVRASPGQGTQSENSCTHFPGNLPNMLRDLRDAFSRVK |
| | TFFQMKDQLDNLLLKESLLEDFKGYLGCQALSEMIQFYLEEVMPQAENQDPD |
| | IKAHVNSLGENLKTLRLRLRRCHRFLPCENKSKAVEQVKNAFNKLQEKGIYK |
| | AMSEFDIFINYIEAYMTMKIRNSGGGGSGGGGSGGGGSGGGGSGGGSLQLLP |
| | SWAITLISVNGIFVICCLTYCFAPRCRERRRNERLRRESVRPV (SEQ ID |
| | NO: 467) |
| | Tethered IFNg |
| Nucleotide | ATGAAATATACAAGTTATATCTTGGCTTTTCAGCTCTGCATCGTTTTGGGTT |
| | CTCTTGGCTGTTACTGCCAGGACCCATATGTAAAAGAAGCAGAAAACCTTAA |
| | GAAATATTTTAATGCAGGTCATTCAGATGTAGCGGATAATGGAACTCTTTTC |
| | TTAGGCATTTTGAAGAATTGGAAAGAGGAGAGTGACAGAAAAATAATGCAGA |
| | GCCAAATTGTCTCCTTTTACTTCAAACTTTTTAAAAACTTTAAAGATGACCA |
| | GAGCATCCAAAAGAGTGTGGAGACCATCAAGGAAGACATGAATGTCAAGTTT |
| | TTCAATAGCAACAAAAAGAAACGAGATGACTTCGAAAAGCTGACTAATTATT |
| | CGGTAACTGACTTGAATGTCCAACGCAAAGCAATACATGAACTCATCCAAGT |
| | GATGGCTGAACTGTCGCCAGCAGCTAAAACAGGGTCAGGCGGCGGTGGTAGT |
| | GGAGGCGGAGGCTCAGGCGGCGGAGGTTCCGGAGGTGGCGGTTCCGGCGGAG |
| | GATCTCTTCAATTGCTGCCTAGCTGGGCCATCACACTGATCTCCGTGAACGG |
| | CATCTTCGTGATCTGCTGCCTGACCTACTGCTTCGCCCCTAGATGCAGAGAG |
| | CGGAGAAGAAACGAGCGGCTGAGAAGAGAAAGCGTGCGGCCTGTG (SEQ |
| | ID NO: 468) |
| Amino acid | MKYTSYILAFQLCIVLGSLGCYCQDPYVKEAENLKKYFNAGHSDVADNGTLF |
| | LGILKNWKEESDRKIMQSQIVSFYFKLFKNFKDDQSIQKSVETIKEDMNVKF |
| | FNSNKKKRDDFEKLTNYSVTDLNVQRKAIHELIQVMAELSPAAKTGSGGGGS |
| | GGGGSGGGGSGGGGSGGGSLQLLPSWAITLISVNGIFVICCLTYCFAPRCRE |
| | RRRNERLRRESVRPV (SEQ ID NO: 469) |
| | Soluble IFNg with mCherry Reporter |
| | (linked with E2A-T2A linker) |
| Nucleotide | ATGAAATATACAAGTTATATCTTGGCTTTTCAGCTCTGCATCGTTTTGGGTT |
| | CTCTTGGCTGTTACTGCCAGGACCCATATGTAAAAGAAGCAGAAAACCTTAA |
| | GAAATATTTTAATGCAGGTCATTCAGATGTAGCGGATAATGGAACTCTTTTC |
| | TTAGGCATTTTGAAGAATTGGAAAGAGGAGAGTGACAGAAAAATAATGCAGA |
| | GCCAAATTGTCTCCTTTTACTTCAAACTTTTTAAAAACTTTAAAGATGACCA |
| | GAGCATCCAAAAGAGTGTGGAGACCATCAAGGAAGACATGAATGTCAAGTTT |
| | TTCAATAGCAACAAAAAGAAACGAGATGACTTCGAAAAGCTGACTAATTATT |
| | CGGTAACTGACTTGAATGTCCAACGCAAAGCAATACATGAACTCATCCAAGT |
| | GATGGCTGAACTGTCGCCAGCAGCTAAAACAGGGAAGCGAAAAAGGAGTCAG |
| | ATGCTGTTTCGAGGTCGAAGAGCATCCCAGGGTAGCGGCCAGTGTACCAACT |
| | ACGCCCTGCTGAAACTGGCCGGCGACGTGGAATCTAATCCTGGACCTGGATC |
| | TGGCGAGGGACGCGGGAGTCTACTGACGTGTGGAGACGTGGAGGAAAACCCT |
| | GGACCTatggtgagcaagggcgaggaggataacatggccatcatcaaggagt |
| | tcatgcgcttcaaggtgcacatggagggctccgtgaacggccacgagttcga |
| | gatcgagggcgagggcgagggccgcccctacgagggcacccagaccgccaag |
| | ctgaaggtgaccaagggtggccccctgcccttcgcctgggacatcctgtccc |
| | ctcagttcatgtacggctccaaggcctacgtgaagcaccccgccgacatccc |
| | cgactacttgaagctgtccttccccgagggcttcaagtgggagcgcgtgatg |
| | aacttcgaggacggcggcgtggtgaccgtgacccaggactcctccctgcagg |
| | acggcgagttcatctacaaggtgaagctgcgcggcaccaacttcccctccga |
| | cggccccgtaatgcagaagaagaccatgggctggcaggcctcctccgagcgg |
| | atgtaccccgaggacggcgccctgaagggcgagatcaagcagaggctgaagc |
| | tgaaggacggcggccactacgacgctgaggtcaagaccacctacaaggccaa |
| | gaagcccgtgcagctgcccggcgcctacaacgtcaacatcaagttggacatc |
| | acctcccacaacgaggactacaccatcgtggaacagtacgaacgcgccgagg |
| | gccgccactccaccggcggcatggacgagctgtacaag (SEQ ID NO: |
| | 470) |
| Amino acid | MKYTSYILAFQLCIVLGSLGCYCQDPYVKEAENLKKYFNAGHSDVADNGTLF |
| | LGILKNWKEESDRKIMQSQIVSFYFKLFKNFKDDQSIQKSVETIKEDMNVKF |
| | FNSNKKKRDDFEKLTNYSVTDLNVQRKAIHELIQVMAELSPAAKTGKRKRSQ |
| | MLFRGRRASQGSGQCTNYALLKLAGDVESNPGPGSGEGRGSLLTCGDVEENP |
| | GPMVSKGEEDNMAIIKEFMRFKVHMEGSVNGHEFEIEGEGEGRPYEGTQTAK |
| | LKVTKGGPLPFAWDILSPQFMYGSKAYVKHPADIPDYLKLSFPEGFKWERVM |
| | NFEDGGVVTVTQDSSLQDGEFIYKVKLRGTNFPSDGPVMQKKTMGWQASSER |
| | MYPEDGALKGEIKQRLKLKDGGHYDAEVKTTYKAKKPVQLPGAYNVNIKLDI |
| | TSHNEDYTIVEQYERAEGRHSTGGMDELYK* (SEQ ID NO: 471) |
| | Tethered IFNg with mCherry Reporter |
| | (linked with E2A-T2A linker) |
| Nucleotide | ATGAAATATACAAGTTATATCTTGGCTTTTCAGCTCTGCATCGTTTTGGGTT |
| | CTCTTGGCTGTTACTGCCAGGACCCATATGTAAAAGAAGCAGAAAACCTTAA |
| | GAAATATTTTAATGCAGGTCATTCAGATGTAGCGGATAATGGAACTCTTTTC |
| | TTAGGCATTTTGAAGAATTGGAAAGAGGAGAGTGACAGAAAAATAATGCAGA |
| | GCCAAATTGTCTCCTTTTACTTCAAACTTTTTAAAAACTTTAAAGATGACCA |
| | GAGCATCCAAAAGAGTGTGGAGACCATCAAGGAAGACATGAATGTCAAGTTT |
| | TTCAATAGCAACAAAAAGAAACGAGATGACTTCGAAAAGCTGACTAATTATT |
| | CGGTAACTGACTTGAATGTCCAACGCAAAGCAATACATGAACTCATCCAAGT |
| | GATGGCTGAACTGTCGCCAGCAGCTAAAACAGGGTCAGGCGGCGGTGGTAGT |
| | GGAGGCGGAGGCTCAGGCGGCGGAGGTTCCGGAGGTGGCGGTTCCGGCGGAG |
| | GATCTCTTCAATTGCTGCCTAGCTGGGCCATCACACTGATCTCCGTGAACGG |
| | CATCTTCGTGATCTGCTGCCTGACCTACTGCTTCGCCCCTAGATGCAGAGAG |
| | CGGAGAAGAAACGAGCGGCTGAGAAGAGAAAGCGTGCGGCCTGTGGGTAGCG |
| | GCCAGTGTACCAACTACGCCCTGCTGAAACTGGCCGGCGACGTGGAATCTAA |
| | TCCTGGACCTGGATCTGGCGAGGGACGCGGGAGTCTACTGACGTGTGGAGAC |
| | GTGGAGGAAAACCCTGGACCTatggtgagcaagggcgaggaggataacatgg |
| | ccatcatcaaggagttcatgcgcttcaaggtgcacatggagggctccgtgaa |
| | cggccacgagttcgagatcgagggcgagggcgagggccgcccctacgagggc |
| | acccagaccgccaagctgaaggtgaccaagggtggccccctgcccttcgcct |
| | gggacatcctgtcccctcagttcatgtacggctccaaggcctacgtgaagca |
| | ccccgccgacatccccgactacttgaagctgtccttccccgagggcttcaag |
| | tgggagcgcgtgatgaacttcgaggacggcggcgtggtgaccgtgacccagg |
| | actcctccctgcaggacggcgagttcatctacaaggtgaagctgcgcggcac |
| | caacttcccctccgacggccccgtaatgcagaagaagaccatgggctggcag |
| | gcctcctccgagcggatgtaccccgaggacggcgccctgaagggcgagatca |
| | agcagaggctgaagctgaaggacggcggccactacgacgctgaggtcaagac |
| | cacctacaaggccaagaagcccgtgcagctgcccggcgcctacaacgtcaac |
| | atcaagttggacatcacctcccacaacgaggactacaccatgtggaacagta |
| | cgaacgcgccgagggccgccactccaccggcggcatggacgagctgtacaag |
| | (SEQ ID NO: 472) |
| Amino acid | MKYTSYILAFQLCIVLGSLGCYCQDPYVKEAENLKKYFNAGHSDVADNGTLF |
| | LGILKNWKEESDRKIMQSQIVSFYFKLFKNFKDDQSIQKSVETIKEDMNVKF |
| | FNSNKKKRDDFEKLTNYSVTDLNVQRKAIHELIQVMAELSPAAKTGSGGGGS |
| | GGGGSGGGGSGGGGSGGGSLQLLPSWAITLISVNGIFVICCLTYCFAPRCRE |
| | RRRNERLRRESVRPVGSGQCTNYALLKLAGDVESNPGPGSGEGRGSLLTCGD |
| | VEENPGPMVSKGEEDNMAIIKEFMRFKVHMEGSVNGHEFEIEGEGEGRPYEG |
| | TQTAKLKVTKGGPLPFAWDILSPQFMYGSKAYVKHPADIPDYLKLSFPEGFK |
| | WERVMNFEDGGVVTVTQDSSLQDGEFIYKVKLRGTNFPSDGPVMQKKTMGWQ |
| | ASSERMYPEDGALKGEIKQRLKLKDGGHYDAEVKTTYKAKKPVQLPGAYNVN |
| | IKLDITSHNEDYTIVEQYERAEGRHSTGGMDELYK* (SEQ ID NO: |
| | 473) |
| | Tethered IL-10 with mCherry Reporter |
| | (linked with E2A-T2A linker) |
| Nucleotide | ATGCACAGCTCAGCACTGCTCTGTTGCCTGGTCCTCCTGACTGGGGTGAGGG |
| | CCAGCCCAGGCCAGGGCACCCAGTCTGAGAACAGCTGCACCCACTTCCCAGG |
| | CAACCTGCCTAACATGCTTCGAGATCTCCGAGATGCCTTCAGCAGAGTGAAG |
| | ACTTTCTTTCAAATGAAGGATCAGCTGGACAACTTGTTGTTAAAGGAGTCCT |
| | TGCTGGAGGACTTTAAGGGTTACCTGGGTTGCCAAGCCTTGTCTGAGATGAT |
| | CCAGTTTTACCTGGAGGAGGTGATGCCCCAAGCTGAGAACCAAGACCCAGAC |
| | ATCAAGGCGCATGTGAACTCCCTGGGGGAGAACCTGAAGACCCTCAGGCTGA |
| | GGCTACGGCGCTGTCATCGATTTCTTCCCTGTGAAAACAAGAGCAAGGCCGT |
| | GGAGCAGGTGAAGAATGCCTTTAATAAGCTCCAAGAGAAAGGCATCTACAAA |
| | GCCATGAGTGAGTTTGACATCTTCATCAACTACATAGAAGCCTACATGACAA |
| | TGAAGATACGAAACTCAGGCGGCGGTGGTAGTGGAGGCGGAGGCTCAGGCGG |
| | CGGAGGTTCCGGAGGTGGCGGTTCCGGCGGAGGATCTCTTCAATTGCTGCCT |
| | AGCTGGGCCATCACACTGATCTCCGTGAACGGCATCTTCGTGATCTGCTGCC |
| | TGACCTACTGCTTCGCCCCTAGATGCAGAGAGCGGAGAAGAAACGAGCGGCT |
| | GAGAAGAGAAAGCGTGCGGCCTGTGGGTAGCGGCCAGTGTACCAACTACGCC |
| | CTGCTGAAACTGGCCGGCGACGTGGAATCTAATCCTGGACCTGGATCTGGCG |
| | AGGGACGCGGGAGTCTACTGACGTGTGGAGACGTGGAGGAAAACCCTGGACC |
| | Tatggtgagcaagggcgaggaggataacatggccatcatcaaggagttcatg |
| | cgcttcaaggtgcacatggagggctccgtgaacggccacgagttcgagatcg |
| | agggcgagggcgagggccgcccctacgagggcacccagaccgccaagctgaa |
| | ggtgaccaagggtggccccctgcccttcgcctgggacatcctgtcccctcag |
| | ttcatgtacggctccaaggcctacgtgaagcaccccgccgacatccccgact |
| | acttgaagctgtccttccccgagggcttcaagtgggagcgcgtgatgaactt |
| | cgaggacggcggcgtggtgaccgtgacccaggactcctccctgcaggacggc |
| | gagttcatctacaaggtgaagctgcgcggcaccaacttcccctccgacggcc |
| | ccgtaatgcagaagaagaccatgggctggcaggcctcctccgagcggatgta |
| | ccccgaggacggcgccctgaagggcgagatcaagcagaggctgaagctgaag |
| | gacggcggccactacgacgctgaggtcaagaccacctacaaggccaagaagc |
| | ccgtgcagctgcccggcgcctacaacgtcaacatcaagttggacatcacctc |
| | ccacaacgaggactacaccatcgtggaacagtacgaacgcgccgagggccgc |
| | cactccaccggcggcatggacgagctgtacaag (SEQ ID NO: 474) |
| Amino acid | MHSSALLCCLVLLTGVRASPGQGTQSENSCTHFPGNLPNMLRDLRDAFSRVK |
| | TFFQMKDQLDNLLLKESLLEDFKGYLGCQALSEMIQFYLEEVMPQAENQDPD |
| | IKAHVNSLGENLKTLRLRLRRCHRFLPCENKSKAVEQVKNAFNKLQEKGIYK |
| | AMSEFDIFINYIEAYMTMKIRNSGGGGSGGGGSGGGGSGGGGSGGGSLQLLP |
| | SWAITLISVNGIFVICCLTYCFAPRCRERRRNERLRRESVRPVGSGQCTNYA |
| | LLKLAGDVESNPGPGSGEGRGSLLTCGDVEENPGPMVSKGEEDNMAIIKEFM |
| | RFKVHMEGSVNGHEFEIEGEGEGRPYEGTQTAKLKVTKGGPLPFAWDILSPQ |
| | FMYGSKAYVKHPADIPDYLKLSFPEGFKWERVMNFEDGGVVTVTQDSSLQDG |
| | EFIYKVKLRGTNFPSDGPVMQKKTMGWQASSERMYPEDGALKGEIKQRLKLK |
| | DGGHYDAEVKTTYKAKKPVQLPGAYNVNIKLDITSHNEDYTIVEQYERAEGR |
| | HSTGGMDELYK* (SEQ ID NO: 475) |
| | Truncated human IFNg |
| Nucleotide | ATGAAATATACAAGTTATATCTTGGCTTTTCAGCTCTGCATCGTTTTGGGTT |
| | CTCTTGGCTGTTACTGCCAGGACCCATATGTAAAAGAAGCAGAAAACCTTAA |
| | GAAATATTTTAATGCAGGTCATTCAGATGTAGCGGATAATGGAACTCTTTTC |
| | TTAGGCATTTTGAAGAATTGGAAAGAGGAGAGTGACAGAAAAATAATGCAGA |
| | GCCAAATTGTCTCCTTTTACTTCAAACTTTTTAAAAACTTTAAAGATGACCA |
| | GAGCATCCAAAAGAGTGTGGAGACCATCAAGGAAGACATGAATGTCAAGTTT |
| | TTCAATAGCAACAAAAAGAAACGAGATGACTTCGAAAAGCTGACTAATTATT |
| | CGGTAACTGACTTGAATGTCCAACGCAAAGCAATACATGAACTCATCCAAGT |
| | GATGGCTGAACTGTCGCCAGCAGCTAAAACAGGG (SEQ ID NO: 476) |
| Amino acid | MKYTSYILAFQLCIVLGSLGCYCQDPYVKEAENLKKYFNAGHSDVADNGTLF |
| | LGILKNWKEESDRKIMQSQIVSFYFKLFKNFKDDQSIQKSVETIKEDMNVKF |
| | FNSNKKKRDDFEKLTNYSVTDLNVQRKAIHELIQVMAELSPAAKTG (SEQ |
| | ID NO: 477) |
| | Linker |
| Nucleotide | TCAGGCGGCGGTGGTAGTGGAGGCGGAGGCTCAGGCGGCGGAGGTTCCGGAG |
| | GTGGCGGTTCCGGCGGAGGATCTCTTCAA (SEQ ID NO: 478) |
| Amino acid | SGGGGSGGGGSG (SEQ ID NO: 479) |
| | B7-1 TM Domain |
| Nucleotide | TTGCTGCCTAGCTGGGCCATCACACTGATCTCCGTGAACGGCATCTTCGTGA |
| | TCTGCTGCCTGACCTACTGCTTCGCCCCTAGATGCAGAGAGCGGAGAAGAAA |
| | CGAGCGGCTGAGAAGAGAAAGCGTGCGGCCTGTG (SEQ ID NO: 480) |
| Amino acid | LLPSWAITLISVNGIFVICCLTYCFAPRCRERRRNERLRRESVRPV (SEQ |
| | ID NO: 481) |
| | IRF7-PEST |
| Nucleotide | ATGGCCTTGGCTCCTGAGAGGGCAGCCCCACGCGTGCTGTTCGGAGAGTGGC |
| | TCCTTGGAGAGATCAGCAGCGGCTGCTATGAGGGGCTGCAGTGGCTGGACGA |
| | GGCCCGCACCTGTTTCCGCGTGCCCTGGAAGCACTTCGCGCGCAAGGACCTG |
| | AGCGAGGCCGACGCGCGCATCTTCAAGGCCTGGGCTGTGGCCCGCGGCAGGT |
| | GGCCGCCTAGCAGCAGGGGAGGTGGCCCGCCCCCCGAGGCTGAGACTGCGGA |
| | GCGCGCCGGCTGGAAAACCAACTTCCGCTGCGCACTGCGCAGCACGCGTCGC |
| | TTCGTGATGCTGCGGGATAACTCGGGGGACCCGGCCGACCCGCACAAGGTGT |
| | ACGCGCTCAGCCGGGAGCTGTGCTGGCGAGAAGGCCCAGGCACGGACCAGAC |
| | TGAGGCAGAGGCCCCCGCAGCTGTCCCACCACCACAGGGTGGGCCCCCAGGG |
| | CCATTCCTGGCACACACACATGCTGGACTCCAAGCCCCAGGCCCCCTCCCTG |
| | CCCCAGCTGGTGACAAGGGGGACCTCCTGCTCCAGGCAGTGCAACAGAGCTG |
| | CCTGGCAGACCATCTGCTGACAGCGTCATGGGGGGCAGATCCAGTCCCAACC |
| | AAGGCTCCTGGAGAGGGACAAGAAGGGCTTCCCCTGACTGGGGCCTGTGCTG |
| | GAGGCCCAGGGCTCCCTGCTGGGGAGCTGTACGGGTGGGCAGTAGAGACGAC |
| | CCCCAGCCCCGGGCCCCAGCCCGCGGCACTAACGACAGGCGAGGCCGCGGCC |
| | CCAGAGTCCCCGCACCAGGCAGAGCCGTACCTGTCACCCTCCCCAAGCGCCT |
| | GCACCGCGGTGCAAGAGCCCAGCCCAGGGGCGCTGGACGTGACCATCATGTA |
| | CAAGGGCCGCACGGTGCTGCAGAAGGTGGTGGGACACCCGAGCTGCACGTTC |
| | CTATACGGCCCCCCAGACCCAGCTGTCCGGGCCACAGACCCCCAGCAGGTAG |
| | CATTCCCCAGCCCTGCCGAGCTCCCGGACCAGAAGCAGCTGCGCTACACGGA |
| | GGAACTGCTGCGGCACGTGGCCCCTGGGTTGCACCTGGAGCTTCGGGGGCCA |
| | CAGCTGTGGGCCCGGCGCATGGGCAAGTGCAAGGTGTACTGGGAGGTGGGCG |
| | GACCCCCAGGCTCCGCCAGCCCCTCCACCCCAGCCTGCCTGCTGCCTCGGAA |
| | CTGTGACACCCCCATCTTCGACTTCAGAGTCTTCTTCCAAGAGCTGGTGGAA |
| | TTCCGGGCACGGCAGCGCCGTGGCTCCCCACGCTATACCATCTACCTGGGCT |
| | TCGGGCAGGACCTGTCAGCTGGGAGGCCCAAGGAGAAGAGCCTGGTCCTGGT |
| | GAAGCTGGAACCCTGGCTGTGCCGAGTGCACCTAGAGGGCACGCAGCGTGAG |
| | GGTGTGTCTTCCCTGGATAGCAGCAGCCTCAGCCTCTGCCTGTCCAGCGCCA |
| | ACAGCCTCTATGACGACATCGAGTGCTTCCTTATGGAGCTGGAGCAGCCCGC |
| | CAGCCACGGCTTTCCGCCTGAGGTTGAAGAGCAAGCCGCCGGTACATTGCCT |
| | ATGTCCTGCGCACAAGAAAGCGGTATGGACCGGCACCCAGCCGCTTGTGCTT |
| | CAGCTCGCATCAACGTC (SEQ ID NO: 498) |
| Amino acid | MALAPERAAPRVLFGEWLLGEISSGCYEGLQWLDEARTCFRVPWKHFARKDL |
| | SEADARIFKAWAVARGRWPPSSRGGGPPPEAETAERAGWKTNFRCALRSTRR |
| | FVMLRDNSGDPADPHKVYALSRELCWREGPGTDQTEAEAPAAVPPPQGGPPG |
| | PFLAHTHAGLQAPGPLPAPAGDKGDLLLQAVQQSCLADHLLTASWGADPVPT |
| | KAPGEGQEGLPLTGACAGGPGLPAGELYGWAVETTPSPGPQPAALTTGEAAA |
| | PESPHQAEPYLSPSPSACTAVQEPSPGALDVTIMYKGRTVLQKVVGHPSCTF |
| | LYGPPDPAVRATDPQQVAFPSPAELPDQKQLRYTEELLRHVAPGLHLELRGP |
| | QLWARRMGKCKVYWEVGGPPGSASPSTPACLLPRNCDTPIFDFRVFFQELVE |
| | FRARQRRGSPRYTIYLGFGQDLSAGRPKEKSLVLVKLEPWLCRVHLEGTQRE |
| | GVSSLDSSSLSLCLSSANSLYDDIECFLMELEQPASHGFPPEVEEQAAGTLP |
| | MSCAQESGMDRHPAACASARINV (SEQ ID NO: 499) |
| | PEST degron |
| Nucleotide | AGCCACGGCTTTCCGCCTGAGGTTGAAGAGCAAGCCGCCGGTACATTGCCTA |
| | TGTCCTGCGCACAAGAAAGCGGTATGGACCGGCACCCAGCCGCTTGTGCTTC |
| | AGCTCGCATCAACGTC (SEQ ID NO: 500) |
| Amino acid | SHGFPPEVEEQAAGTLPMSCAQESGMDRHPAACASARINV (SEQ ID NO: |
| | 501) |

TABLE 8
Additional Engineered Polarization-Specific Enhancers

| SB | SEQ | | Polarization |
|---|---|---|---|
| Construct | ID | | State |
| ID | NO | Enhancer DNA Sequence | Specificity |
| SB09935/ | 420 | TTATAGTATTAGGTCAATGTGTTATTATTTTAAAGTGTTC | M2 |
| SB11755/ | | TGTGTTCAACGAGGCTCAGAAAATGCTGGCCTCAAGACCT | |
| SB11771/ | | CTGAGGACCTCTGTCATTTCCAGAAACATGTGGACTTTTT | |
| SB11779/ | | GCTTCAAAAAGCATTCCTTGAGTAATCTGTTCTGCGGGGT | |
| SB12984/ | | ACATGACATTTATACTTCTCTGAGAATATGGTGTTTGTTT | |
| SB12987 | | TGAAATGCCTTATCTGTCTCTGCAAATGCTGTCAATACAT | |
| Enhancer 1 | | CTGTGTTAGCACAGGACGTTCCAGAAGACAGGAAGCTTAC | |
| | | ATGTGATCTGTTCTCGTAATATGTCTAGGGCTATGACGTA | |
| | | TTCAGTGCTAATGGACCTCAGTTACAGCACAAGGAGACAA | |
| | | GGTGGAGCAATAGCATTTTGTTATTGGGAACTAGGCTACA | |
| | | CTTACTTCCTTATGCACAATAAGAGGCATCCACCTAAAGA | |
| | | ATGATGCAAAATAGACAATCAACACAGAAATTAGGCAGAC | |
| | | ACTATCACTCTAAGAGCAGATGTTGGAGTCGCCTTGATTA | |
| | | GACTTTGGATCCTGGCTTTCGCACTTA | |
| SB09955/ | 421 | ATAGGTTTAGAGCCTTGCTCCTGCAGCTTCAAAAACAAGG | M2 |
| Enhancer 2 | | AGGCAAAAATTGAGATTGAATATAGCACAGAGTGTGGGTG | |
| | | ACACCAAACCACGGACTGAGTTGATAGGCTGCAGGGAAGG | |
| | | AAGTGAAAAGTTCACCTTGTCCTTCCGTCTAACAGAGGAC | |
| | | TGCTGGTTTTGCAGAGGTTTGCTTTTTTTTTTTTTTCCAT | |
| | | TCAATAAAAATAGCTCGGTGGGACAACATATTATAGCATT | |
| | | CGTGGAAATAAAAACTAGGAACCACATCATAGGATGTTCT | |
| | | GCTCCAAACTTGGCGCTGATCCCCCAGGCAGCTCCAAGTT | |
| | | CCCTCTGATTCAGGCAGCAGCGACGGAGGCCCTCCCCTGT | |
| | | CCCAGGTGGGACCCGGCCTTCAGAGAGGAAGGGAGGTGGT | |
| | | GGTATGAGGCATCAGCTGTTTCCTTTGCCTGTGGGCCAAG | |
| | | ATCCTTGCCATGGCAGAAATACAACTTTGTCCCCAGTGTC | |
| | | TGCCTTCTGCACTGTAAAAAGCAAGTTTCCATCACAAAGG | |
| | | GTTGCGATTCAGGAAACCAGGGTTAGAGGCAAACGGGTCT | |
| | | TATTTGAAAATGGTAAAACCTGGCCAGGCCTTGGACTTCC | |
| | | TGCATTGGTTGCTCTCCTGGGACCTGGGACCAGCCCAGCA | |
| | | GGGTGGGGTGGGGCAGCAATGCAGGTTACAGGATGTGAAA | |
| | | GTGGGGACTGAGGTTAGGAAAAAGAATAAAAGGCTGTGAC | |
| | | CAGAACACACCCAGCAGAGGCCATGTGCAGTTCTCTTCTT | |
| | | CAGGGTTTCCTGTGGGTACACTGAGAACATTGTGTTCTGG | |
| | | CTGAGAACTTATCCGGCGGAGCCTGCTGCATACGTGGCCC | |
| | | CTGGGTCCTAGTGCTGCCTCTTACCTAGCCTCAGGCCTGG | |
| | | CTCCCCTGCCTGCACCTGCGTGCAGCCCACTGTATGACCC | |
| | | AGGGAGGCTGTCTCCATTGTGTCCTC | |
| SB09956/ | 422 | GCCCATTTGAGTCCCAAGATCTGCCAACAGATCCTTCCTC | M2 |
| Enhancer 3 | | CACGTCTGAAATGATGTTGCGTCAGGCAGAGGCACACAGC | |
| | | CCCGACTGTCTCCAGTACAGACATTTCTTCAGGCCACTTC | |
| | | ACCTTCCTCTGCTTACAGCTCCCTCCGGGCAGGCGCGGGG | |
| | | CCTGCCTGTGCTCATCCTGCAGGCAGAGAACATTGCCAGG | |
| | | TCAGCAGTATACACCGTCCCCTCAGACACAAGGAGTATTA | |
| | | TTTAGTCTAGAAACTCTCAAAGGAAATATCCGCTTGCACG | |
| | | TGAAAAATCTCTGGAATTTCTCTGTTCCTCTCTGTCCAAC | |
| | | GTCCTTGTATCTGCAAAACCTCCTTGAAAAGCTCCTGTGT | |
| | | ACAACTGCTGCCTTGTTCGGGCCCGACTGCTTCAGGCCTG | |
| | | ACCCCAGGGCACTTCTGTCCCAGCCAGTGTCATTCAGAGC | |
| | | GATTTCTGCTCCTCCAAGACGGGCTGTGCCCATGGTGAAG | |
| | | GAAAGAGGAGGGGTAAGGGGAGAAGGAGGTGGGGGGATGA | |
| | | GGTGAAGAACAGAGAGGGAGGAGAAGGAGGGAGAGGAAGG | |
| | | GGAGGACCCTCTGAAG | |
| SB09959/ | 423 | AATTAGTGCATTGGTTTATCAGGAACTCTTTTCTTGGTGC | M2 |
| SB11758/ | | TCTATTCTGCTCTCCATGACCTTGGTTGCCTGAGTGATAC | |
| Enhancer 4 | | ATCCTTTAATTTGTTCCTACATCTTGCTTGGATTTTCCCC | |
| | | TGCTCATCTCTGTCTCTGCTTATACAGGATGTGTTTTATT | |
| | | GCCACACAGACTTAGCTGGTAGCCTCTGTGGCATCATCCA | |
| | | ACACTGATGTCCCTCAGTCAACTCTAGAACACCCATAGGT | |
| | | GGCCTCTGTAAGAGACGTTTCCAGGAACAGAATGCCAAAC | |
| | | ATACTGTTTATCAGAGTTCTTGAGTTGCACAATGGGAGGA | |
| | | AATTGTTGCTTAGGATTTCAAAGGACACCAGAGAATTTCA | |
| | | ACACACACCTGGAATGGTGCCCTGTTGCTTGTCCAGTCAA | |
| | | TGCCAAGTTAATGATGGGAAGGAATAGCTGGTGTGTCACG | |
| | | TGATTGCCAAGCACTGTCGCAAATCCTTCCTGAGGTCAAC | |
| | | ACTCCCAACCCCGCCAAACTCTACAGCTCACAAATCAGCT | |
| | | GCAAGTGGAGCATAAAAAATGCAACATTGTGGAATTAACT | |
| | | ACTTATGGGCCACA | |
| SB09963/ | 424 | CTATCAGCAATTCATTGAGCCTTGTGATTTCCTGGGTGAT | M2 |
| Enhancer 5 | | AGAGAAACAGGCCATTGAAACGAGTCCCTAGTCCTAGCTC | |
| | | ATGGCATCCAAATAGCAAGATCAGTGACATAGACTATTCA | |
| | | ATGTGAACTAGGAGGAAATCTGCCTCATCACCATTAAAAA | |
| | | TGATGAGACTACATGGTCAGTGGGTGTTAAGCAACATTGC | |
| | | CAGGAAAAACGGGCAGGCCAAGGTTCAGATGGGAAATGTT | |
| | | GGCATGCAATGTTGATTTAAGTAATCTGGATAAAAAATAC | |
| | | TTTTCATGCTGAGACACGAGTCATGAGGCGCTAGAATATA | |
| | | GGGATAACTTCCTTTCAACTCTTTAAAAAGTACATATGGT | |
| | | AAATATTTGCTGATTTAGACTATGTAGAGAAGAATCAAAA | |
| | | TGAATCAGTAAAACATCCAAATAGGGAAGTATGGGTGTGG | |
| | | AAAGCATGTATTTTGAGCAGAATTTAACGAAGCCGACCAA | |
| | | GGGTTCCCTATGGAAGGAACTATGGAATGAAGGACTGAAC | |
| | | ATTTGATTAAAGAGCAGTT | |
| SB09964/ | 425 | TTGTCTTGTCGTGATTGCAAGTGGGATGTGGTCAATTAGT | M2 |
| SB11760/ | | CGAGCTATGCTTTATGCATCCAGACCCTTATAATTGAGGA | |
| SB11776/ | | GCTGGCATTCTTGCCTGTCTGGGCAGGCAATTGCTCAGCA | |
| SB11784/ | | GGGAGCCTCATGGCCAGACAGGCAAGAGGGAAGAGGGCAA | |
| SB11752/ | | TGTGGTTGCTCAATGAGCCTTTAGAACAAATTGATCCGGA | |
| Enhancer 6 | | CTGTTTGTGGTTCCAAAAGCCACCCAGTACTTGCTATGTG | |
| | | GTTTTCCTTGTGGACAATTGGGCAATGGGAGGGGGTGGGG | |
| | | ACAGGAAACCTTAACCTCTGCCTAGCCACTCACAGCAAGA | |
| | | TGAGAGGCAACTGCTCAGTGCGTGTCTTCAGCTCATGCCC | |
| | | TCTTTCGCCTGTCTTGGCCCATCTTCTTCAGGGATGGGAA | |
| | | CCAGCATGATGAGGGAGAAGTTACTCAAGGGGCCGTCTTC | |
| | | TTGGCTTCAGCCCTGTGGAAGGCCTAGTGGGCTTTGGACC | |
| | | TCACAGCCAATCAGGTGAGTCACTTGAGCAACTGAAGAAC | |
| | | CAAGACAGCTTATGGCTGGGCTCCAGGATGGGGAGGTCCC | |
| | | AGGAGAGGGCAGAGCAGGAGAGAAGGCTCTAAGGTTAATT | |
| | | GGAAAGAACAGAACAGTGAAAACC | |
| SB09965/ | 426 | ATAAATTTCTTGATAGAATTTAAATGTTAAGTGTCCTGAA | M2 |
| SB11785/ | | ATGTGTTTACTTCTGGATCAGAAATGAGAATGTACTGAAA | |
| Enhancer 7 | | CAGGAAGTCAGCTTCACAGCAGGAAGCAGACCTCAAAGAA | |
| | | ATGTGACTCACATAGTCTTTTGAATGTGCTCCACTTGGGG | |
| | | ACACAATGTTCCCCACAGCTTGCCCATCTCATCCATTCTA | |
| | | ACTTTCCCATGGGACAAAAAGCATCATAACAAAGAATTAG | |
| | | AGGAGA | |
| SB09966/ | 427 | ATCAAAAATTTTGGTCAGACATGAGATCTTCTCCCTGTCA | M2 |
| SB11754/ | | GACTGTCTTTTTATAGACATTGTGTCACAGGTTCTGAAAT | |
| SB12983 | | GTTGAAAGGGTTTTGTTTCCTGACTCAGGCTACTTGATTC | |
| Enhancer 8 | | TGCCTATTCACAGAAGTCTGCAGCACCCACCGAGGCTGGC | |
| | | CACGTGATTGGATATGGCCTTTCTTTAGCTCTGCAGATAT | |
| | | GTGGATATATTCATTTGCTAGGGCTGCAATAACAAATACC | |
| | | ACAGTCTGGGTTGCTTGCACAACAGAAATTTATTGTCTCA | |
| | | CAGCTCTGGAGACTAGAAGTCCAGGATCAAGGTCTTAGCC | |
| | | AATTTGGTTCCTTCTGGGGGCTATGAGAGAGACACTGTTC | |
| | | CATGCCTCTCCTAGCTTCTGGTGGTTGGCTGGTTTGCTGG | |
| | | CCATCTTTGG | |
| SB09846 | 428 | CCACTTCCGGGTTCAGACCACTTCCGGGTTCTCGCCACTT | M2 |
| | | CCGGGTTCGACCCACTTCCGGGTTCCTACCACTTCCGGGT | |
| | | TCACTCCACTTCCGGGTTC | |
| SB10543 | 429 | CTGACCTTTGTGCCATACTAGTCACGTGCCTTCCGAAAAC | M2 |
| | | ACTGCACATTGTTTCCGGTAAATAAACAATTAAAATCTTC | |
| | | TGAGTCAGCA | |
| SB10544 | 430 | TGCACCTGTTACGGTTGTATGTCACGTGCCTTCGGGTAAC | M2 |
| | | ACTGCACATTGTTTCCACACATCTGCTTCCTGTGCTCCAC | |
| | | TGAGTCAGCA | |
| SB10545 | 431 | CTGAGTCAGCAAGGGTGCTTAAGTAAACAAATGCCGACTT | M2 |
| | | ATTGGAAAAACTGCTAACAGTTTCATTATGACTCACGGTC | |
| | | CACTTCCGGTCT | |
| SB10552 | 432 | TTTCACACCTTTCGTTTAACAATGTGCAGTGTTATGTATC | M2 |
| | | TGAGTCAGCAAATAACCACCACTTCCGGTCTAGACGCGAT | |
| | | TTTTTCCA | |
| SB10555 | 433 | ACGTCAATGGGTGGAGTATTTACGGTAAACTGCCCACTTG | M2 |
| | | GCAGTACATCAAGTGTATCATATGCCAAGTACGCCCCCTA | |
| | | TTGACGTCAATGACG | |
| SB11267 | 434 | AGGCGGAAGTGAGCTCTGAGTCATAATGAAACTGACCAGA | M2 |
| | | TTTCCAGGAAAACGATGACCTGCAGTGACCTCTGTGTATA | |
| | | TTGCACAATC | |
| SB11268 | 435 | TATTGCACAATCTAAGCATTTTTCCTGGAAAAGATTGCCT | M2 |
| | | GATGACTCATCTATTATGCTATCACGTCACCACTGGGTCT | |
| | | CACTTCCGCCT | |
| SB11279 | 436 | TATTGCACAATCAGCTAGCATTTCCAGGAAAAACGGAACA | M2 |
| | | AGGCGGAAGTGTTTTCGTCAGATGACTCATCTATCGGCGA | |
| | | AACAGCTGTT | |
| SB11288 | 437 | TATTGCACAATCTAGCTATATTTCCAGGAAAAGTATAGCA | M2 |
| | | TGCGTGGGCGTAGATCTGATGTCACGTGACTTCACGATCT | |
| | | CACTTCCTGCTC | |
| SB11293 | 438 | GAGCAGGAAGTGAGTCGCCATTCCTTTGATCTTTTGACTG | M2 |
| | | CAGTCACGTGACTGAGGAGCATTTCCAGGAAAACCGAACT | |
| | | AAGGCGGAAGTG | |
| SB11297 | 439 | TGACCTGCAGTGACCTCTGTGAGTCATAATGAAACTGAGG | M2 |
| | | TCTCACTTCCTGCTCTCCGTTTGATGAGTCATCTTCGCGC | |
| | | AGATTGTGCAATA | |
| SB10560 | 440 | CCGAAACCGAAACTAATACAATCAAAGGTCAGTTGAGATA | M1 |
| | | GAATGGAAAAACCAGCCAGAGTCATAATGAAACTGTACAC | |
| | | ATATGTAAATAT | |
| SB10562 | 441 | AAGGTGTGAAATGCTAGATAATTGTTTATTTAGCTACGAA | M1 |
| | | AGTAAACAAAAAATGTAAAGATGGAAAAACCCTATAGAGT | |
| | | CATAATGAAACTG | |
| SB10564 | 442 | GAGTCATAATGAAACTGAAGGTAAAAGATAAGAAAAAAAC | M1 |
| | | TCCGAAACCGAAACTAAGTGTTTCAAAGGTCAGAAATTCC | |
| | | CAAAATAAACAATT | |
| SB10570 | 443 | AATGGCCCGCCTGGCTGACCGCCCAACGACCCCCGCCCAT | M1 |
| | | TGACGTCAATAATGACGTATGTTCCCATAGTAACGCCAAT | |
| | | AGGGACTTTCCATTG | |

TABLE 9
Exemplary Constitutive Promoter Sequences

| Name | Sequence |
|---|---|
| SFFV | GTAACGCCATTTTGCAAGGCATGGAAAAATACCAAACCAAGAATAGAGAAGTTCAGATC |
| | AAGGGCGGGTACATGAAAATAGCTAACGTTGGGCCAAACAGGATATCTGCGGTGAGCAG |
| | TTTCGGCCCCGGCCCGGGGCCAAGAACAGATGGTCACCGCAGTTTCGGCCCCGGCCCGA |
| | GGCCAAGAACAGATGGTCCCCAGATATGGCCCAACCCTCAGCAGTTTCTTAAGACCCAT |
| | CAGATGTTTCCAGGCTCCCCCAAGGACCTGAAATGACCCTGCGCCTTATTTGAATTAAC |
| | CAATCAGCCTGCTTCTCGCTTCTGTTCGCGCGCTTCTGCTTCCCGAGCTCTATAAAAGA |
| | GCTCACAACCCCTCACTCGGCGCGCCAGTCCTCCGACAGACTGAGTCGCCCGGG (SEQ |
| | ID NO: 444) |
| EFS | GGATCTGCGATCGCTCCGGTGCCCGTCAGTGGGCAGAGCGCACATCGCCCACAGTCCCC |
| | GAGAAGTTGGGGGGAGGGGTCGGCAATTGAACCGGTGCCTAGAGAAGGTGGCGCGGGGT |
| | AAACTGGGAAAGTGATGTCGTGTACTGGCTCCGCCTTTTTCCCGAGGGTGGGGGAGAAC |
| | CGTATATAAGTGCAGTAGTCGCCGTGAACGTTCTTTTTCGCAACGGGTTTGCCGCCAGA |
| | ACACAGCTGAAGCTTCGAGGGGCTCGCATCTCTCCTTCACGCGCCCGCCGCCCTACCTG |
| | AGGCCGCCATCCACGCCGGTTGAGTCGCGTTCTGCCGCCTCCCGCCTGTGGTGCCTCCT |
| | GAACTGCGTCCGCCGTCTAGGTAAGTTTAAAGCTCAGGTCGAGACCGGGCCTTTGTCCG |
| | GCGCTCCCTTGGAGCCTACCTAGACTCAGCCGGCTCTCCACGCTTTGCCTGACCCTGCT |
| | TGCTCAACTCTACGTCTTTGTTTCGTTTTCTGTTCTGCGCCGTTACAGATCCAAGCTGT |
| | GACCGGCGCCTAC (SEQ ID NO: 445) |

TABLE 10
Exemplary Minimal Promoter Sequences

| Name | Sequence |
|---|---|
| YBTATA | TCTAGAGGGTATATAATGGGGGCCA (SEQ ID NO: 446) |
| (also | |
| referred to | |
| herein as | |
| minPRO1) | |
| minCMV | taggcgtgtacggtgggaggcctatataagcagagctcgtttagtgaaccgtcag |
| (also | atcgcctgga (SEQ ID NO: 447) |
| referred to | |
| herein as | |
| minPRO2) | |
| minTK (also | ttcgcatattaaggtgacgcgtgtggcctcgaacaccgagcgaccctgcagcgac |
| referred to | ccgcttaa (SEQ ID NO: 448) |
| herein as | |
| minPRO3) | |
| SCP3 (also | AGGTCTATATAAGCAGAGCTCGTTTAGTGAACCGTCAGTCCGCCTGGAGACCTCG |
| referred to | AGCCGAGTGGTCGTGCCTCCATAGAA (SEQ ID NO: 449) |
| herein as | |
| minPRO4) | |
| YB-SCP3 | TCTAGAGGGTATATAAGCAGAGCTCGTTTAGTGAACCGTCAGTCCGCCTGGAGAC |
| (also | CTCGAGCCGAGTGGTCGTGCCTCCATAGAA (SEQ ID NO: 450) |
| referred to | |
| herein as | |
| minPRO5) | |
| minCMV | CGGATCAACTtaggcgtgtacggtgggaggcctatataagcagagctcgtttagt |
| with | gaaccgtcagatcgcctggaGGATCCgcgtcaagtggagcaaggcaggtggacag |
| flanking | tCCTGCAGGggagctacc (SEQ ID NO: 451) |
| spacers | |
| minTK with | CGGATCAACTttcgcatattaaggtgacgcgtgtggcctcgaacaccgagcgacc |
| flanking | ctgcagcgacccgcttaaGGATCCgcgtcaagtggagcaaggcaggtggacagtC |
| spacers | CTGCAGGggagctacc (SEQ ID NO: 452) |
| SCP3 with | CGGATCAACTAGGTCTATATAAGCAGAGCTCGTTTAGTGAACCGTCAGTCCGCCT |
| flanking | GGAGACCTCGAGCCGAGTGGTCGTGCCTCCATAGAAGGATCCgcgtcaagtggag |
| spacers | caaggcaggtggacagtCCTGCAGGggagctacc (SEQ ID NO: 453) |
| YB-SCP3 | CGGATCAACTTCTAGAGGGTATATAAGCAGAGCTCGTTTAGTGAACCGTCAGTCC |
| with | GCCTGGAGACCTCGAGCCGAGTGGTCGTGCCTCCATAGAAGGATCCgcgtcaagt |
| flanking | ggagcaaggcaggtggacagtCCTGCAGGggagctacc (SEQ ID NO: 454) |
| spacers | |
| YBTATA | CGGATCAACTTCTAGAGGGTATATAATGGGGGCCAGAACACATCGCTAAGCGAA |
| with | AGCTAAGCTCCAGTACAGGGGCTTTTGGTGACCGGTCAGAGGAAAGGTGAATCC |
| flanking | AATGGGATCCgcgtcaagtggagcaaggcaggtggacagtCCTGCAGGggagct |
| spacers | acc (SEQ ID NO: 455) |

### EXAMPLES

Below are examples of specific embodiments for carrying out the present invention. The examples are offered for illustrative purposes are not intended to limit the scope of the present invention in any way.

Efforts have been made to ensure accuracy with respect to numbers used (e.g., amounts, temperatures, etc.), but some experimental error and deviation should, of course, be allowed for.

### Example 1: Generation of Macrophage Polarization Reporter Constructs and Assessment of Same

Engineered macrophage promoters were screened for and identified as polarization state-specific promoters in two ways: 1) re-engineering of native promoter sequences from the human genome and 2) rationally designing fully engineered promoter sequences.

For method 1), native promoters associated with genes up-regulated in the desired polarization state (based on RNA-seq data) were screened.

### Native Promoter Screening

Candidate native promoters for genes that were significantly up-regulated in the “on” target polarization state compared to the “off” target states were bioinformatically identified, and native promoter candidates were identified from publicly available data sets. RNA sequencing data of macrophages under polarization conditions was generated to identify additional native promoter candidates.

The 2 kb sequence upstream of the transcriptional start site (TSS) or translational start codon in the human genome of each gene of interest was used as the native promoter candidate, as described above. The native promoter candidate was then linked to a fluorescent protein reporter for screening promoter activity (GFP, mCherry, or NanoLuc). When expression was leaky, a PEST-Tag was added to increase protein turnover.

For screening promoter activity, the promoter sequence was linked to a fluorescent reporter to identify potential polarization-state specific activity. The promoter-reporter constructs were lentivirally transduced into primary human macrophages. Briefly, VSV-G pseudotyped lentivirus was generated via co-transfection into HEK293 Ts, collected, and concentrated. After transduction, the cells were unpolarized (M0) or polarized to the M1 or M2c macrophage states. To identify M1-state specific promoters, constructs were identified that turned on higher reporter expression in the M1 state, but not the M2c or M0 states. To identify M2c-state specific promoters, constructs were identified that turned on higher reporter expression in the M2c state, but not the M1 or M0 states. For lentiviral transduction, briefly, individual lentiviral constructs were cloned/synthesized containing the candidate promoters driving GFP or mCherry reporter. At day 0 (D=0), monocytes were isolated from previously frozen primary human peripheral blood mononuclear cell (PBMCs) using CD14+ selection via magnetic activated cell sorting (MACS). From D=0 to D=4, the CD14+ monocytes were differentiated to M0 macrophages in media containing M-CSF. At D=4, the M0 macrophages were passaged into plates for subsequent transduction. At D=5, the M0 macrophages were transduced with lentiviral vectors containing the promoter-reporter construct, and at day=6 the macrophages were kept in M0 state, or polarized to M1 state (with LPS & IFNγ) or M2c state (with IL-10, TGFβ, and dexamethasone). At D=8, all cells were assessed via flow cytometry for promoter activity and/or cell phenotype. Promoter activity was assessed in each polarization state by measuring reporter activity relative to a constitutive control and untransduced control in each polarization state

A variety of payloads were expressed from these promoter constructs for screening. Reporter constructs included the expression of GFP, mCherry, RFP, and secreted NanoLuc. To ascertain whether these promoter constructs were capable of driving phenotypic changes, these promoters were also designed to drive cytokine payloads such as IL-10, IL-4, TGFβ, IFNα, IFNγ, IL-12p70 or TNFα. It is contemplated that alternate payloads for use in such assay include transcription factors, receptors, enzymes, chemokines, antibodies, fragments of antibodies, miRNAs, shRNAs, etc.

These promoter-reporter constructs were then transduced into primary human macrophages according to the methods described above. A constitutive promoter was included in all conditions to normalize for differences in polarization conditions and as a standard for promoter strength.

M1 Promoter Strength was quantified as described below:

M1 Promoter polarization state selective activity was quantified as described below: ( [symbol] )

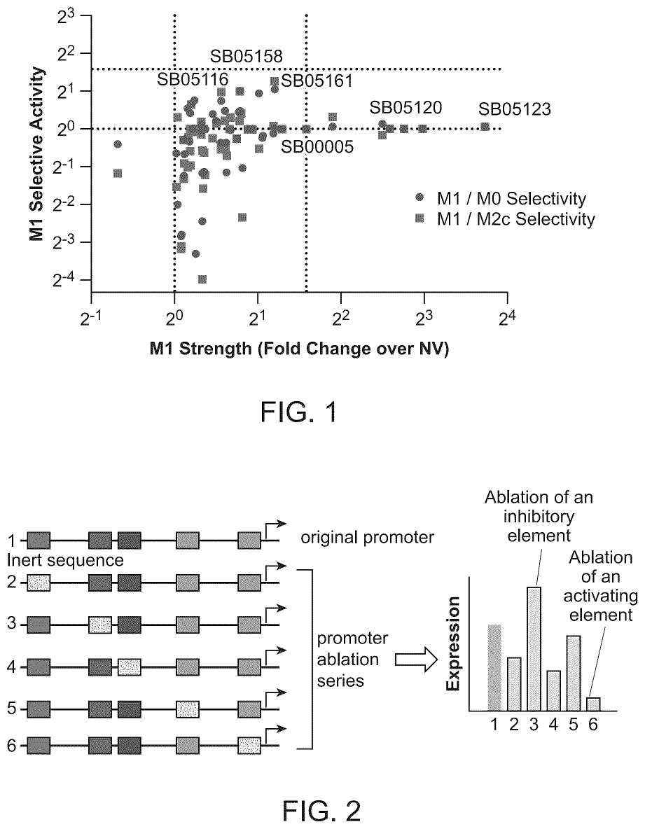

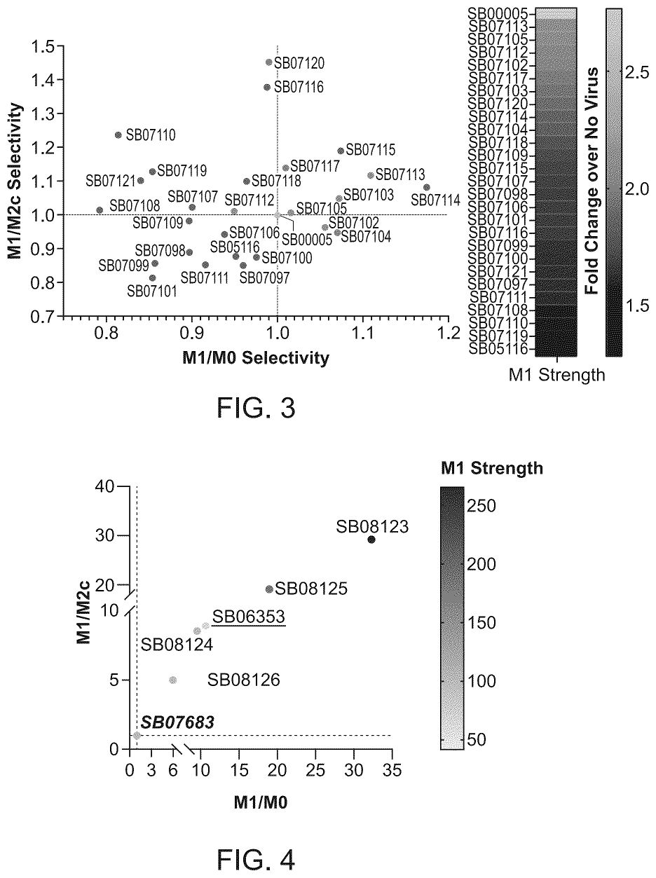

In certain cases, e.g., where native M1 macrophage promoters did not exhibit strong or selective activity (FIG. 1), native promoter sequences were re-engineered into engineered promoters. Bioinformatically-identified transcription factor binding sites in native promoter sequences were then identified and systematically ablated to determine regulatory element activity (FIG. 2). Activity from promoter ablation variants are shown in FIG. 3.

SB07103, SB07113, SB07114, SB07115, and SB07117 ablation variants showed improved M1 selectivity over both M0 SB07116, and SB07110 showed M1 se ( [symbol] ) to SB07101 all showed a decrease in M1 selectivity, indicating the stretch of “neighboring” patches may need to be maintained. SB0005 is a constitutive promoter control that drives high reporter expression regardless of cell state and was used as control.

New engineered promoters were designed: SB08123, SB08124, SB08125, and SB08126. The SB06353 promoter was chosen for follow-up studies, shown in FIG. 4. SB08123 and SB08125 improved selectivity and strength of the original promoter SB06353. SB08123 showed the highest strength and specificity, followed by SB08125. SB08124 showed similar selectivity to the original promoter SB06353 but higher strength.

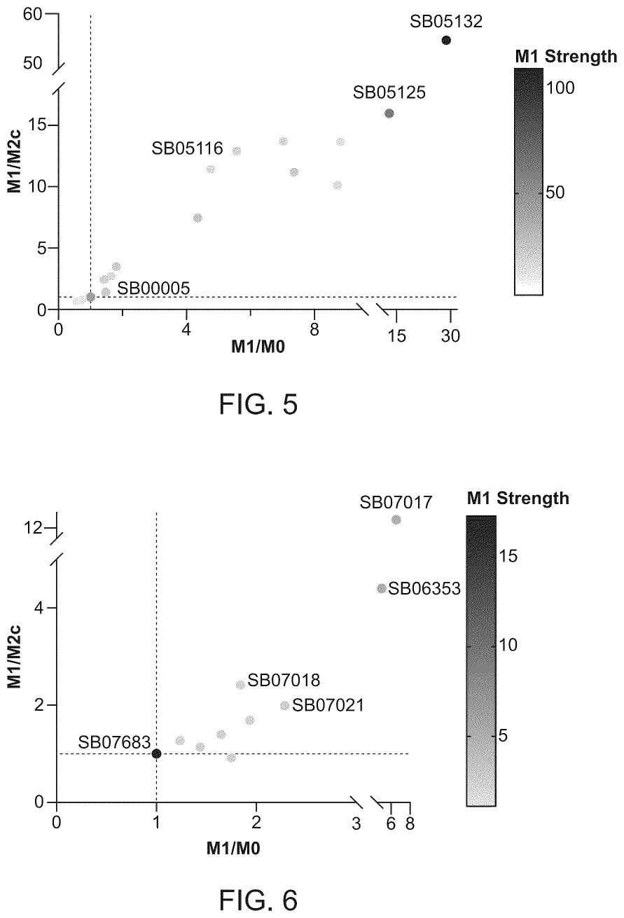

Native promoters were re-screened with VPX lentivirus to identity better promoter hits (FIG. 5). Incorporation of the VPX accessory protein into lentiviral vectors improved promoter performance metrics. SB05132 and SB05125 were identified as new native M1 promoter candidates that could be used as polarization-state specific promoters.

Promoters for non-coding genes also were identified as potential state-specific promoters (FIG. 6). In such genes, the promoter was identified as the 2 kb region upstream of a non-coding RNA. SB07017 showed the highest M1 promoter selectivity, higher than previous positive control SB06353.

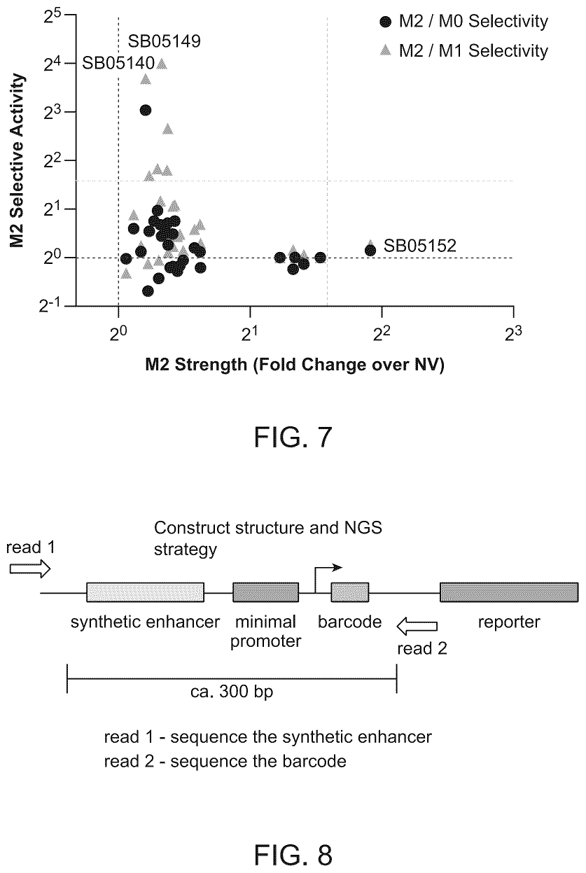

Native candidate M2 promoters showed good selectivity, but poor promoter strength (FIG. 7). SB05140 was highly M2c selective, but very weak; SB05152 was very strong, but leaky; and SB05149 is highly M2c selective, but very weak and only selective over M1 state (not M0).

M2 Promoter Strength was quantified as described below:

$${GFP}_{min} = Minimal GFP Singal \to from No Virus Control$$

$${GFP}_{max} = Minimal GFP Singal \to from SB 00005$$

$${GFP}_{Pro} = Candidate Promoter GFP Signal \to from candidate promoter$$

$$Normalized Change in GFP Expression = \frac{{GFP}_{Pro} - {GFP}_{min}}{{GFP}_{max} - {GFP}_{min}}$$

$$Square brackets [ ] represent values$$

$$in the underscored polarization state$$

$$M 2 Strength = \frac{{[ {GFP}_{Pro} ]}_{M 2}}{{[ {GFP}_{min} ]}_{M 2}}$$

M2 Promoter polarization state selective activity was quantified as described below:

$$M 2 vs . M 0 Selectivity = \frac{{[ \frac{{GFP}_{Pro} - {GFP}_{min}}{{GFP}_{max} - {GFP}_{min}} ]}_{M 2}}{{[ \frac{{GFP}_{Pro} - {GFP}_{min}}{{GFP}_{max} - {GFP}_{min}} ]}_{M 0}}$$

$$M 2 vs . M 1 Selectivity = \frac{{[ \frac{{GFP}_{Pro} - {GFP}_{min}}{{GFP}_{max} - {GFP}_{min}} ]}_{M 2}}{{[ \frac{{GFP}_{Pro} - {GFP}_{min}}{{GFP}_{max} - {GFP}_{min}} ]}_{M 1}}$$

### Engineered Promoter Design

For method 2) engineered promoters were screened based on transcription factors that are upregulated in either the M1 or M2 states. Existing literature data and RNA-sequencing data were mined to identify the most highly expressed transcription factors (TFs) in response to M1 or M2 polarization cues in macrophages. Top transcription factors or enhancers and their corresponding binding sites associated with each polarization state (from both RNA-sequencing and ATAC-sequencing data) were then identified bioinformatically. The top motifs or transcription factor binding sites (TFBS) were combinatorially rearranged into 100 bp engineered enhancers upstream of a minimal promoter (YBTATA) to create an engineered promoter library. TFBS were variably spaced so that the centers of each binding site was optimally spaced to be on the same side of a DNA helical turn. Unique barcodes were designed to be unique to each engineered promoter library member and downstream of the minimal promoter. After screening the combinatorial library of engineered promoters in macrophages, top promoters that had higher expression in one polarization state, but not the other, were identified.

The library was made up out of several segments incorporating TF binding motifs as well as fragments of naturally occurring enhancer sequences, and control sequences.

Construct abundance within the library varied over more than 2 orders of magnitude. 99.7% of the constructs displayed abundances above 10 counts at this sequencing depth of approximately 100 times the library diversity. A graphical schematic is shown in FIG. 8.

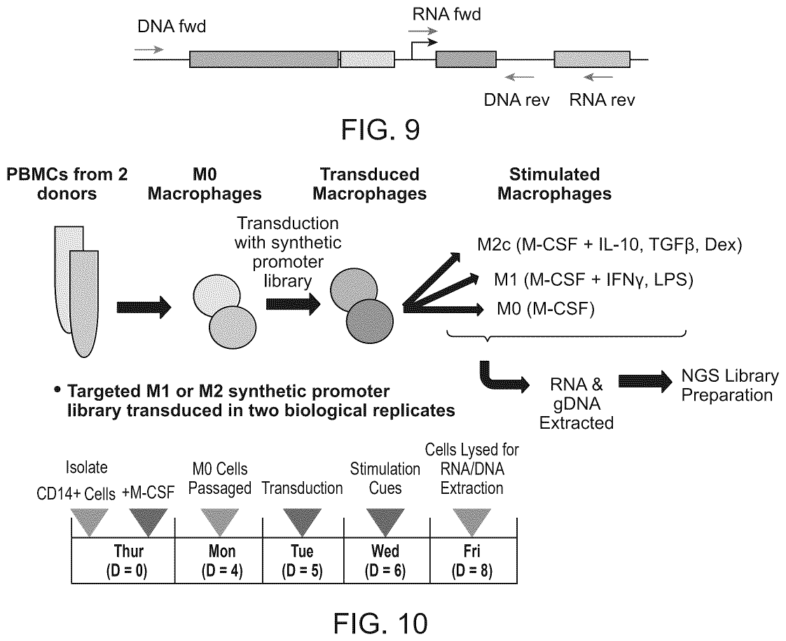

The library of promoters was constructed in lentiviral backbone for transduction. Macrophages were transduced with lentiviral library. gDNA and RNA libraries were prepared from the same samples for NGS with the following primer binding sites depicted in (FIG. 9). Relative promoter activity for each polarization condition was calculated by normalizing RNA counts reads to gDNA reads within a given sample. The workflow is depicted in FIG. 10.

The MPRA libraries exhibited relatively uniform count distribution, as was determined using the below formula:

$$Expression ( \underline{E} ) of individual enhancer$$

$$E = Cn\_RNA / Cn\_DNA$$

$$Cn\_RNA and Cn\_DNA are depth - normalized$$

$$counts from DNA and RNA seq respectively$$

$$Differential expression ( \underline{Ed} ) for$$

$$individual enhancer in different conditions$$

$$Ed = Ex , c 1 / Ex , c 2$$

$$x is a particular enhancer$$

$$c 1 and c 2 are conditions 1 and 2 respectively$$

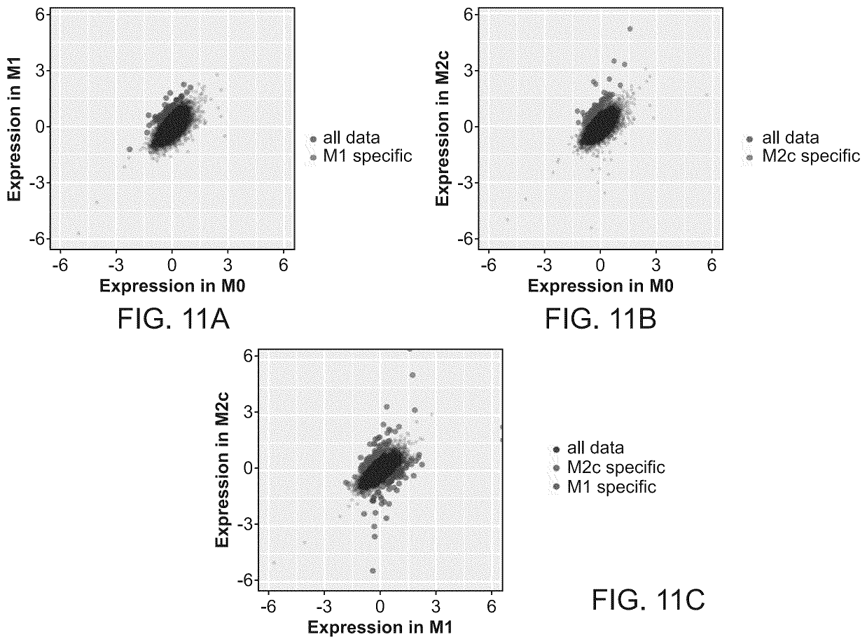

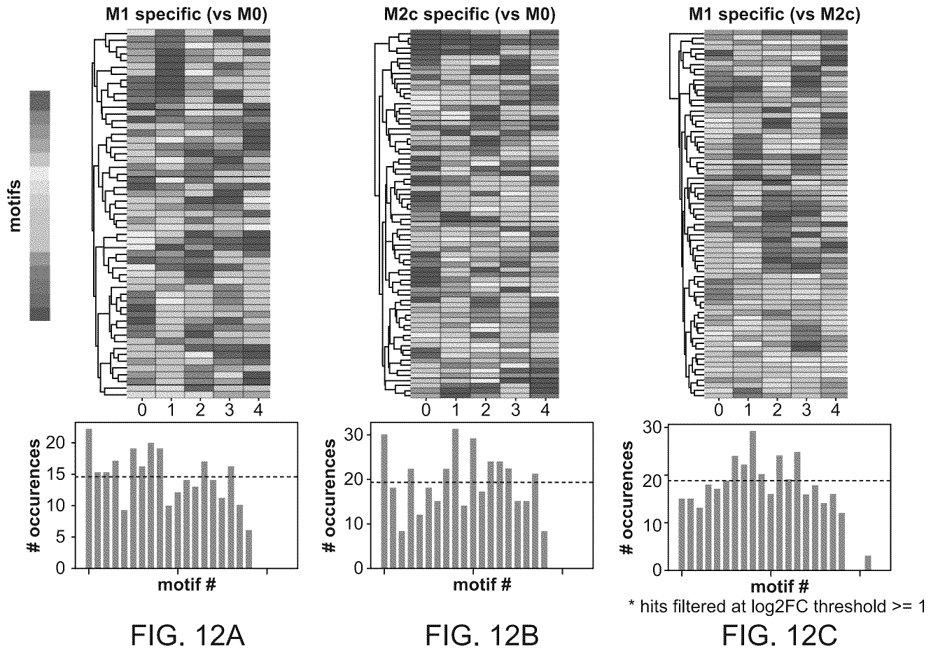

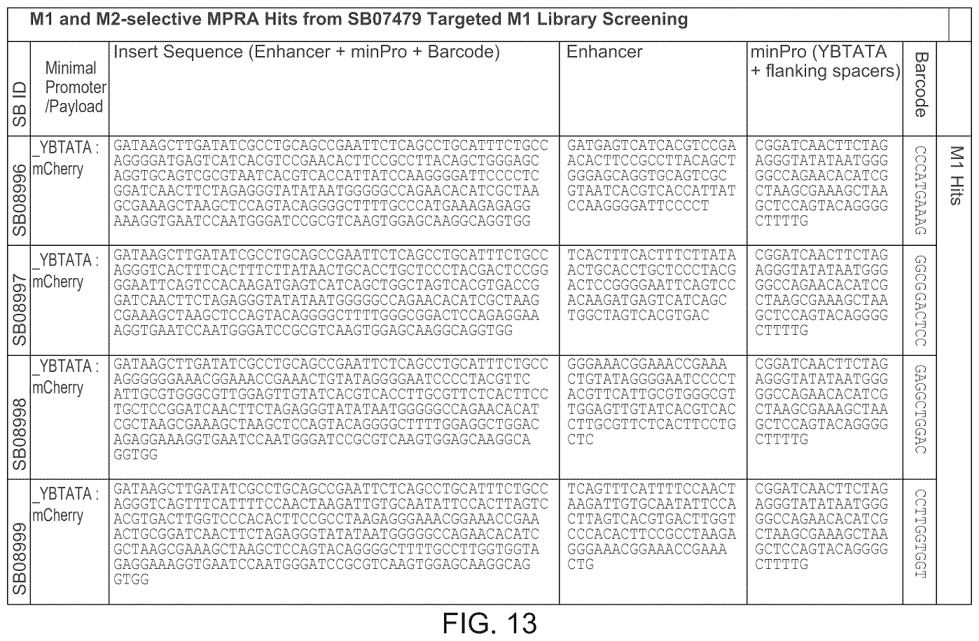

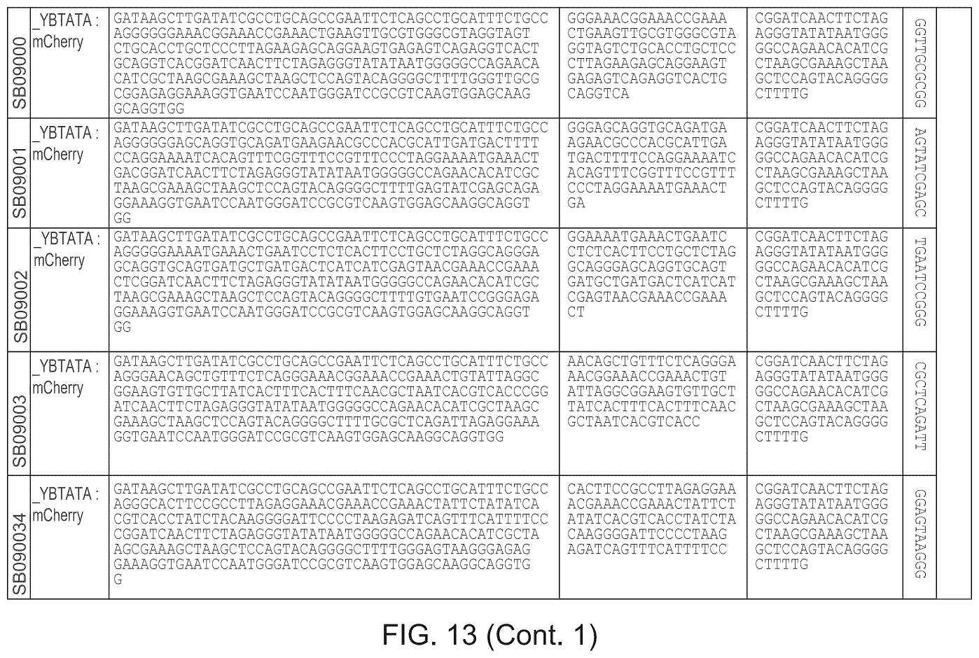

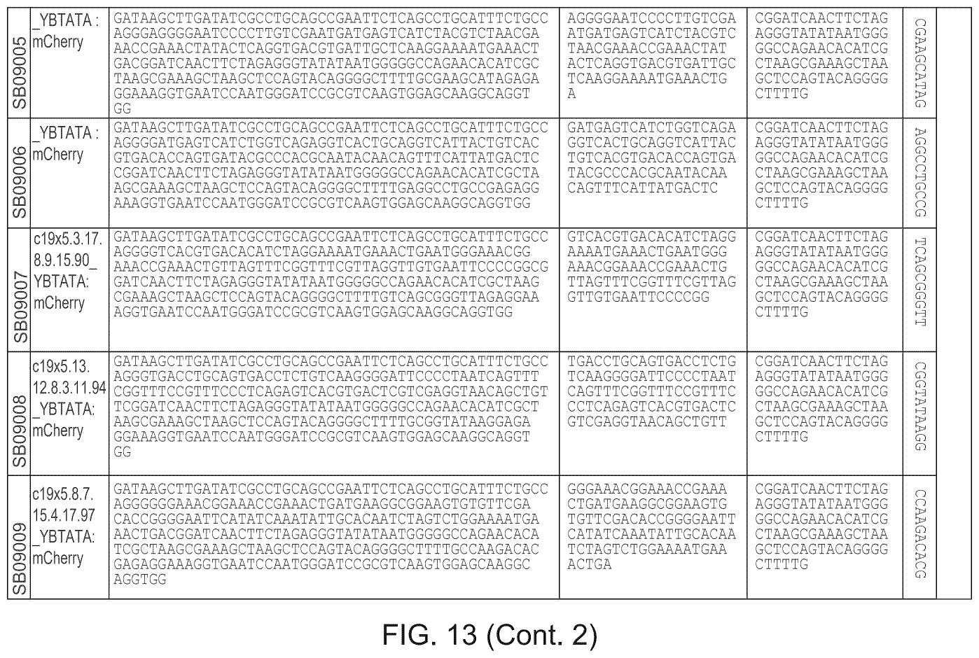

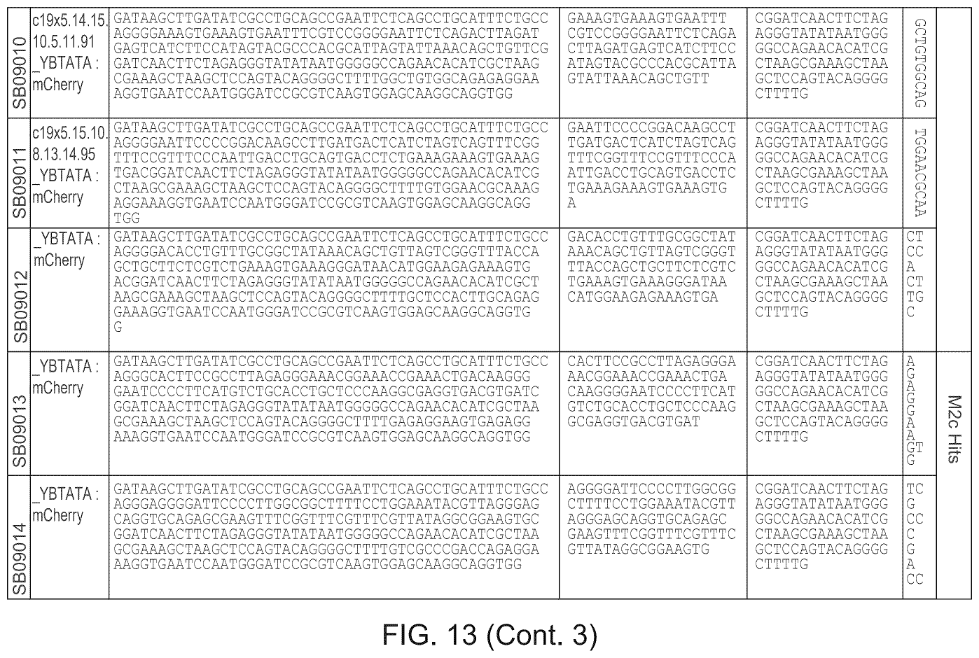

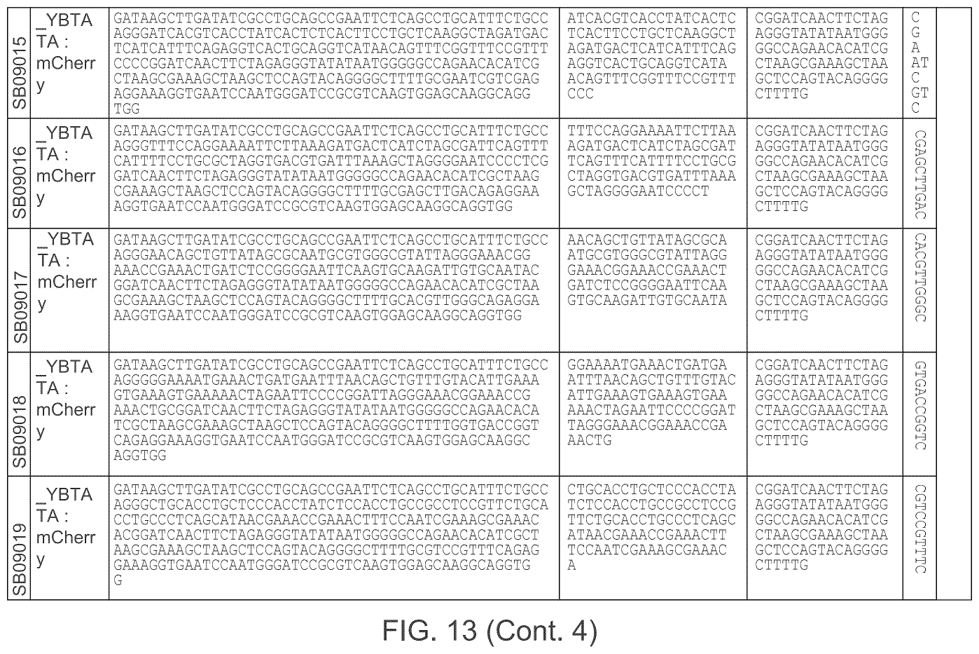

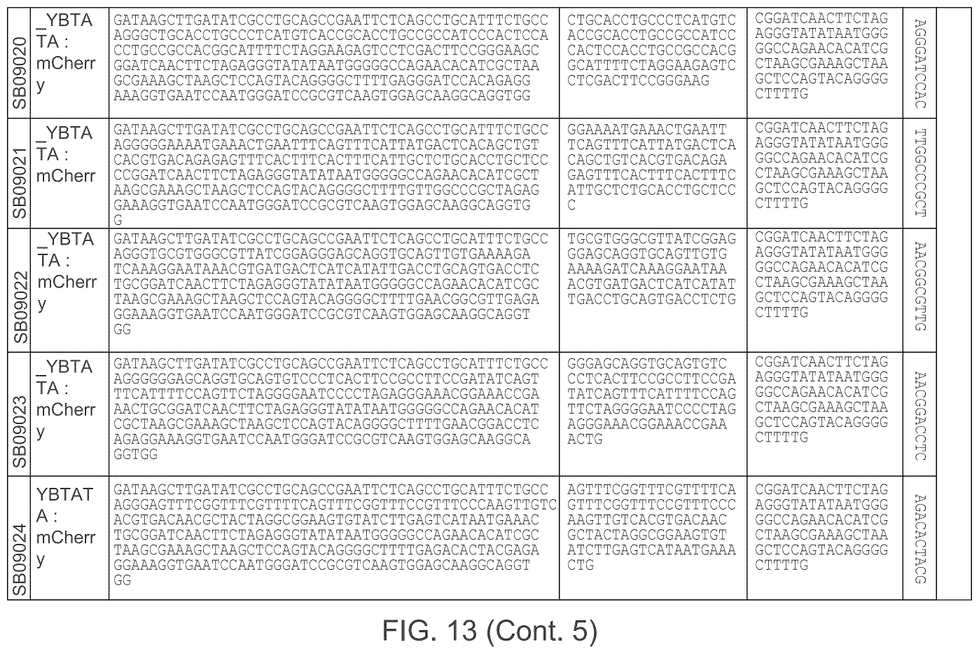

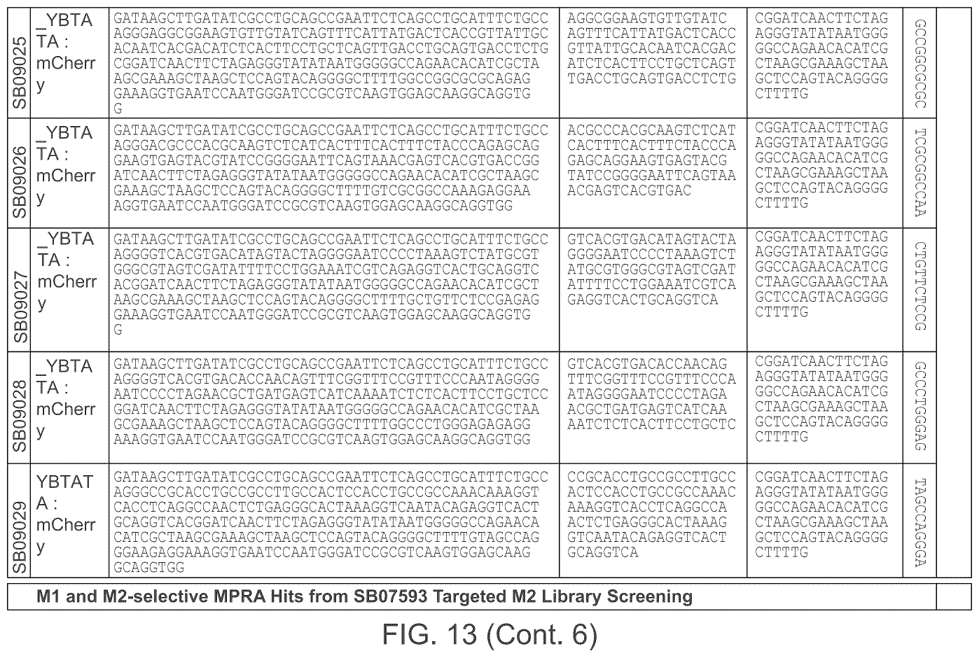

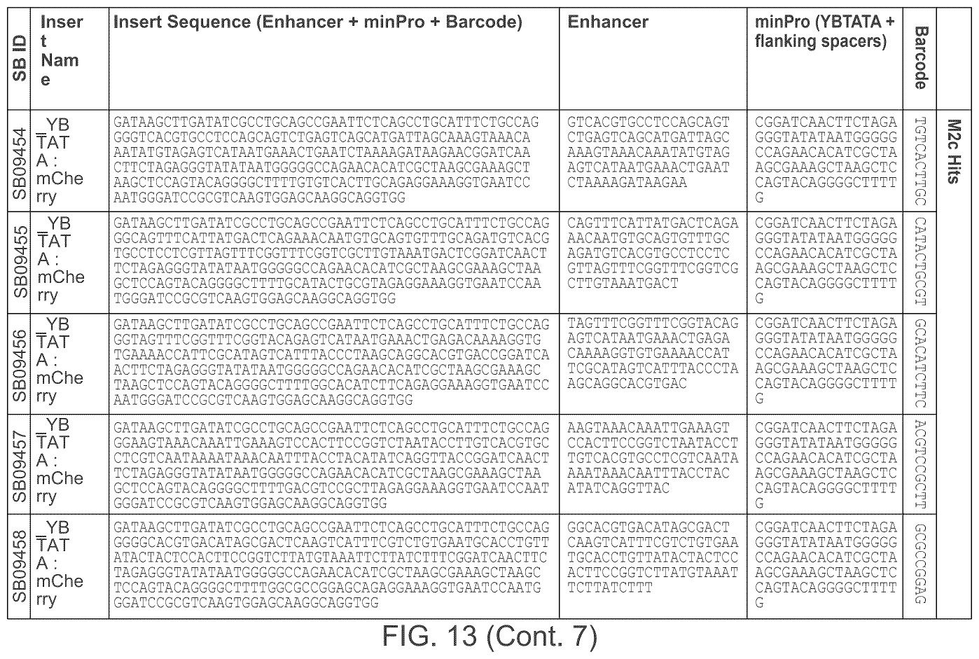

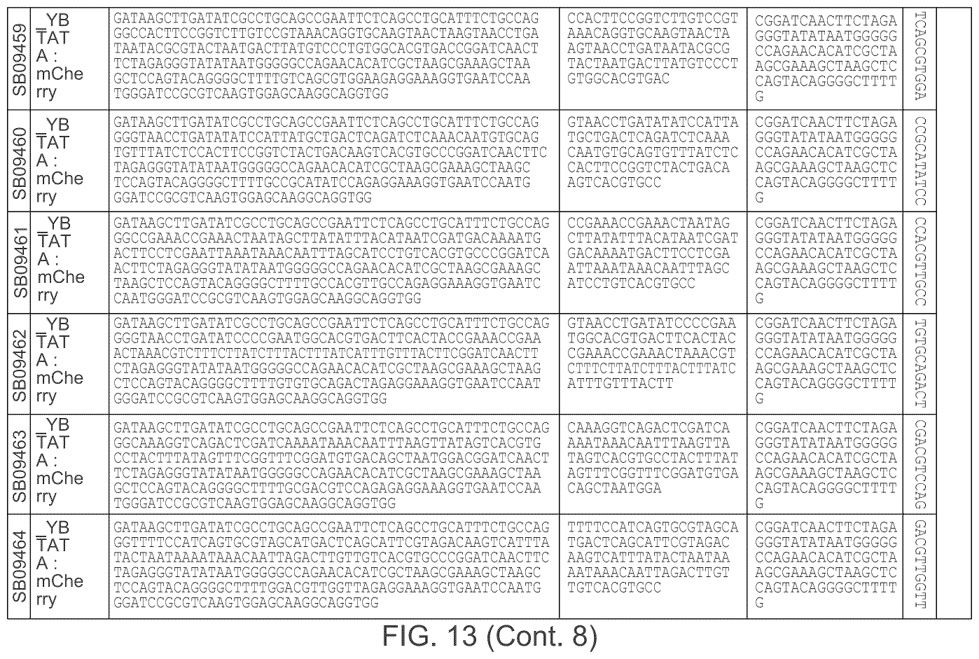

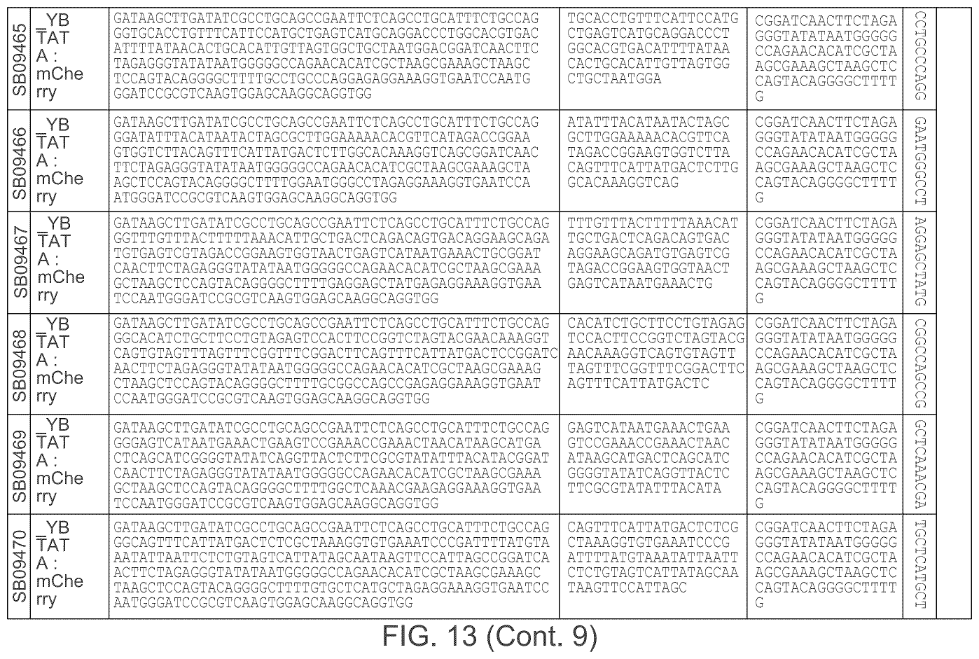

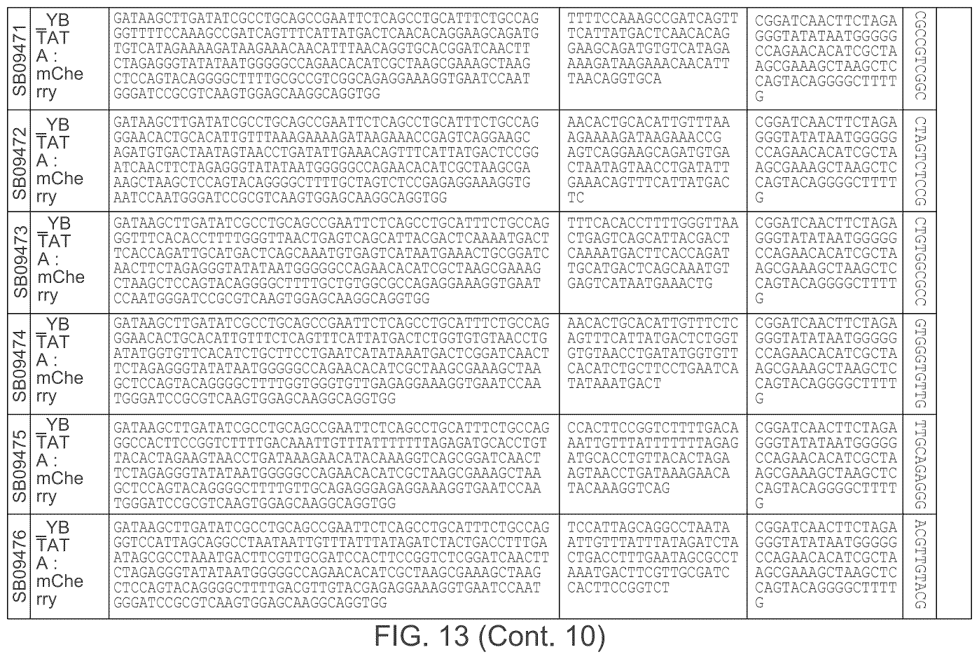

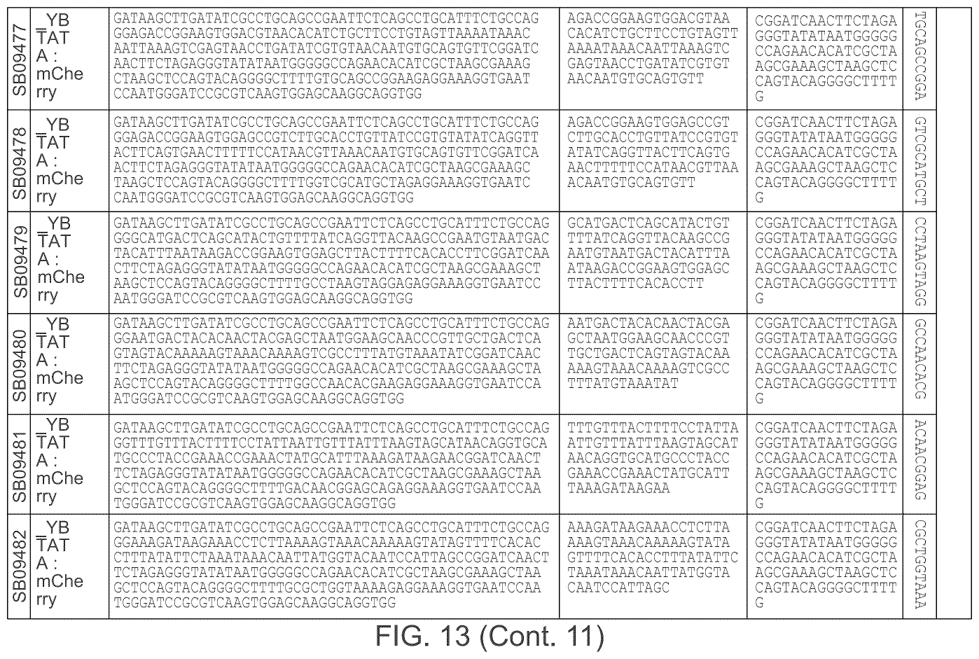

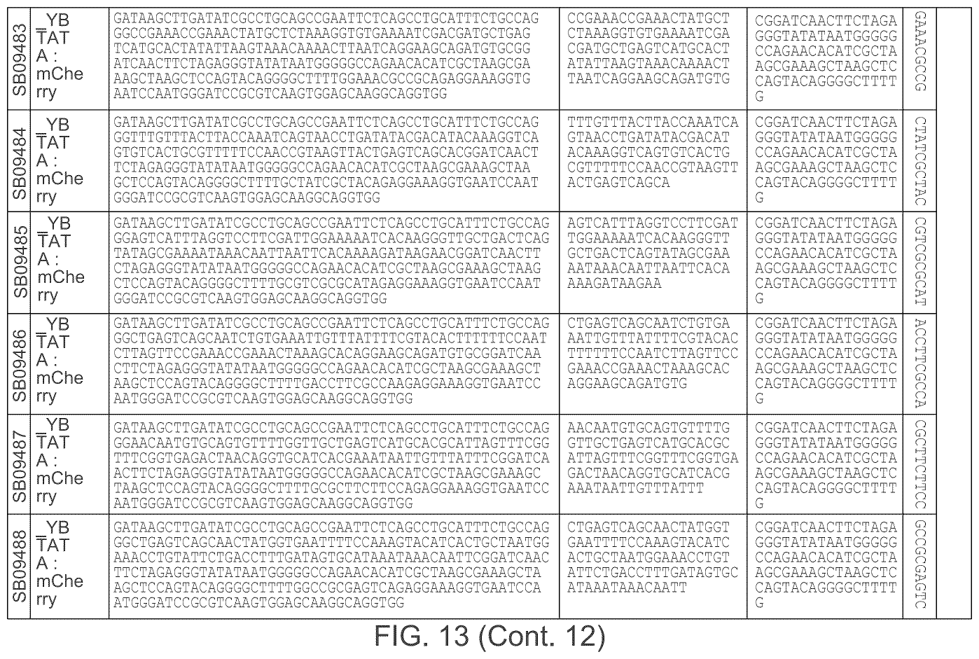

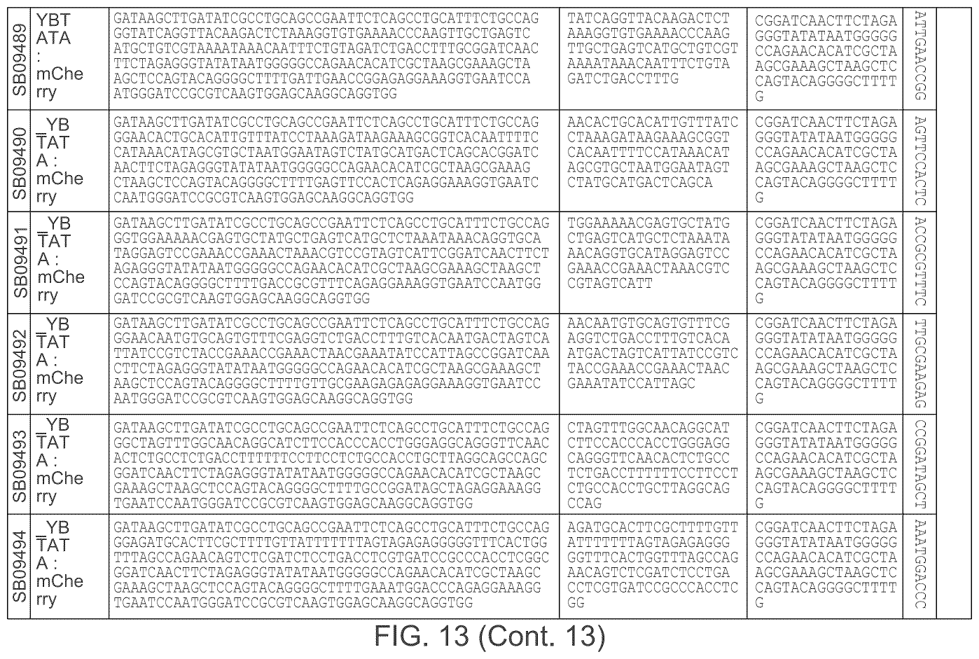

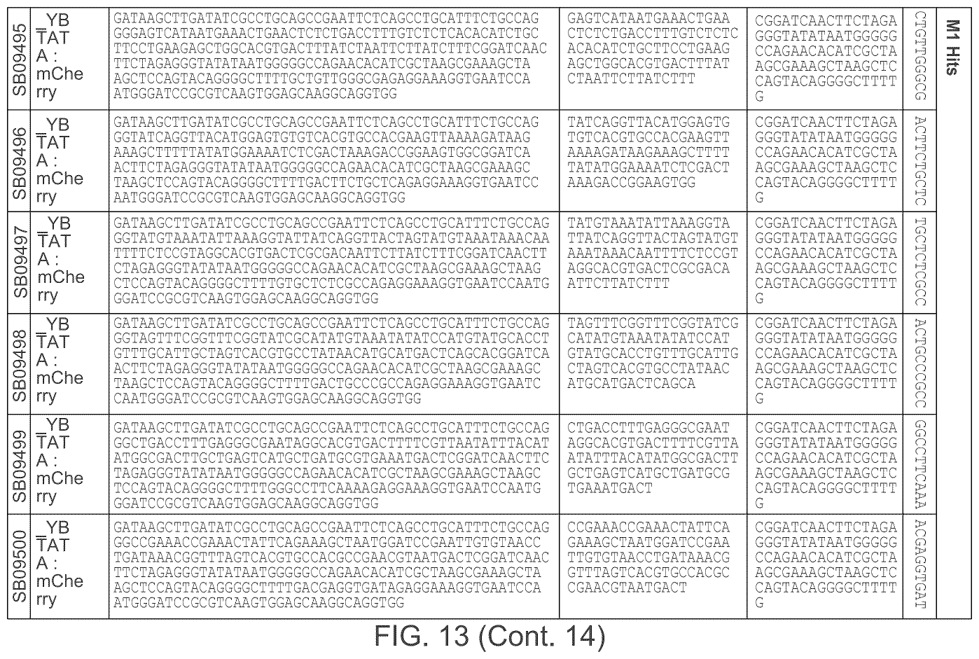

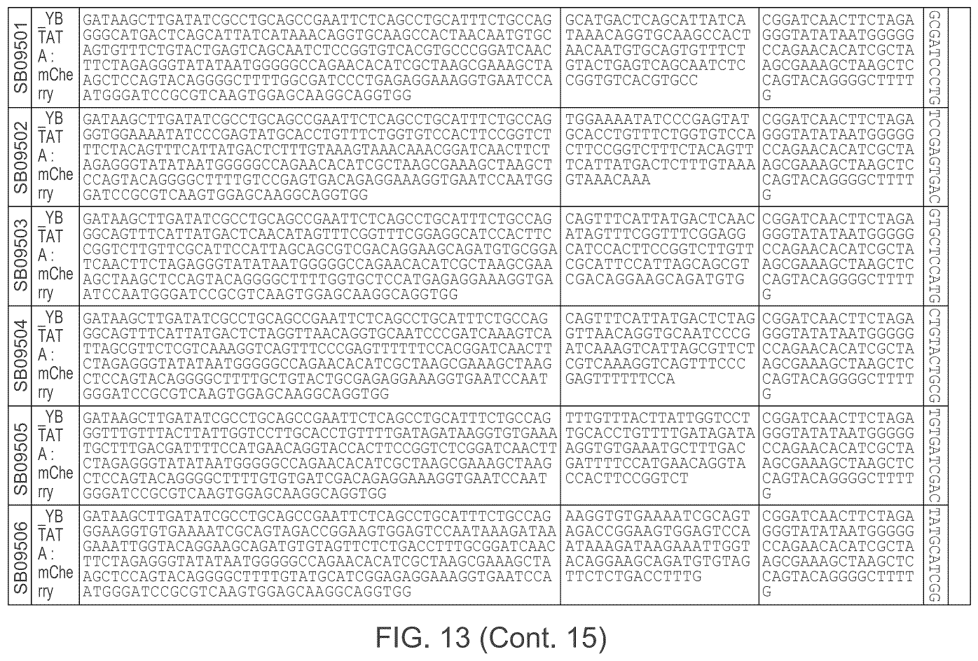

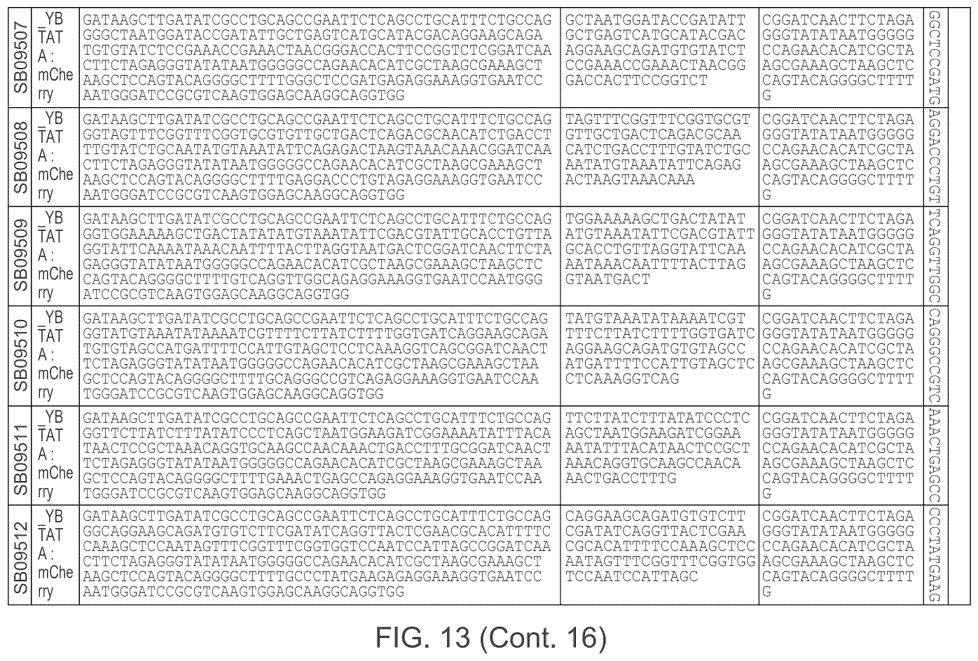

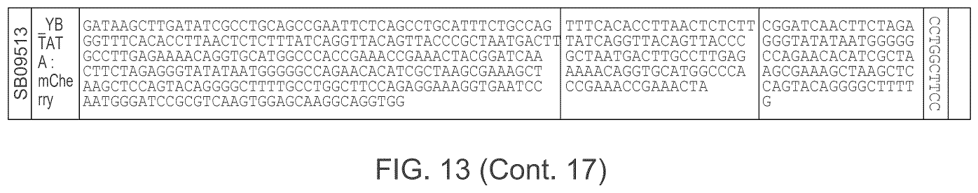

High-throughput MPRA screening yielded engineered promoters that were selective for M1 vs M0 (FIG. 11A), M2c vs M0 (FIG. 11B), and M1 vs M2c macrophage states (FIG. 11C). Interestingly, the MPRA hits displayed distinct patterns of motif enrichment, as shown in FIGS. 12A-12C. Such patterns suggest options for further engineering to enhance activity and selectivity of the polarization-specific promoters. Exemplary promoters identified are shown in FIG. 13, for which the sequences of the enhancers are provided in Table 3, supra.

### Example 2: IDO1 Promoter Ablation Screening

The IDO1 native promoter (native promoter sequence in SB05125 and SB09385) was identified as an M1 specific promoter according to the methods described in Example 1. Ablation studies were conducted to identify regions of the IDO1 promoter that act as potential M1 selective repressive elements and potential M1 selective activating elements. Ablation sites were bioinformatically identified as potential transcription factor binding sites, DNAse hypersensitivity sites, or regions of preferentially open chromatin that may impart promoter activity. Thus, 20 regions of the IDO1 native promoter sequence were targeted for ablation by replacement with inert sequences. Promoter strength and M1 Promoter polarization state selective activity were assessed according to the methods described in Example 1.

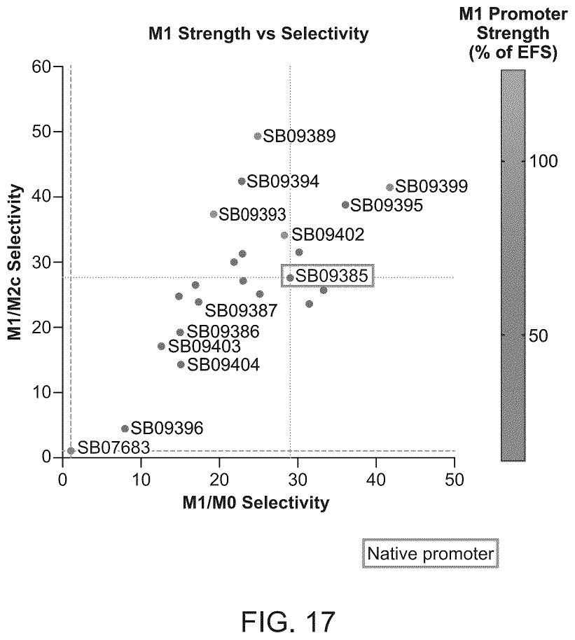

Activity from these promoter ablation variants are shown in FIG. 17. The promoter ablation variants SB09389, SB09393-SB09395, SB09399 and SB09402 demonstrated increased promoter strength & increased M1 selectivity as compared to the native promoter construct SB09385, indicating that these ablated regions represent M1 selective repressive elements. By contrast, the promoter ablation variants SB09386-SB09387, SB09403-SB09404, and SB09396 demonstrated decreased promoter strength & decreased M1 selectivity as compared to the native promoter construct SB09385, indicating that these ablated regions represent M1 selective activating elements.

### Example 3: UBD1 Promoter Ablation Screening

The UBD1 native promoter (native promoter sequence in SB05132 and SB09406) was identified as an M1 specific promoter according to the methods described in Example 1. Ablation studies were conducted to identify regions of the UBD1 promoter that act as potential M1 selective repressive elements and potential M1 selective activating elements. Ablation sites were bioinformatically identified as potential transcription factor binding sites, DNAse hypersensitivity sites, or regions of preferentially open chromatin that may impart promoter activity. Thus, 19 regions of the UBD1 native promoter sequence were targeted for ablation by replacement with inert sequences. Promoter strength and M1 Promoter polarization state selective activity were assessed according to the methods described in Example 1.

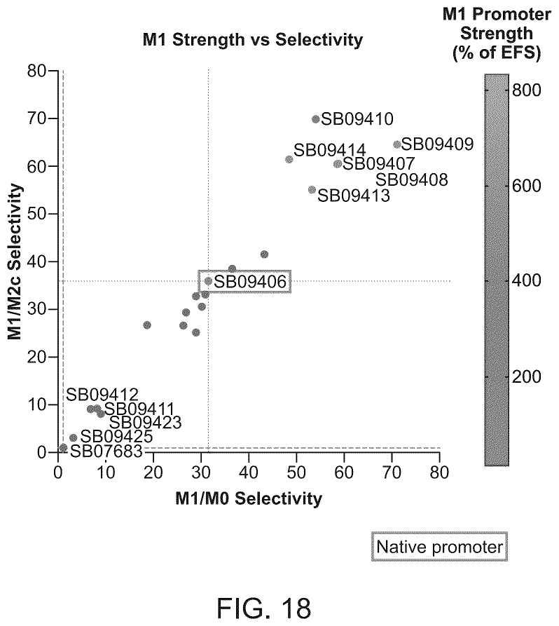

Activity from these promoter ablation variants are shown in FIG. 18. The promoter ablation variants SB09407-SB09410, and SB09413-SB09414 demonstrated increased promoter strength & increased M1 selectivity as compared to the native promoter construct SB09406, indicating that these ablated regions represent M1 selective repressive elements. By contrast, the promoter ablation variants SB09411-SB09412, SB09423 and SB09425 demonstrated decreased promoter strength & decreased M1 selectivity as compared to the native promoter construct SB09406, indicating that these ablated regions represent M1 selective activating elements.

### Example 4: M1 Promoters Nominated from MPRA Library Screening

A targeted, synthetic promoter library was constructed according to the methods described in Example 1 (Engineered promoter design), and screened for promoter activity via a high-throughput MPRA-based assay. The library is composed of over ten thousand constructs with combinatorial arrays of transcription factor binding site motifs designed to be active in the M2 state. From the MPRA-assay, 15 hits from were selected from sequencing results to have higher activity in the M1 state and were then clonally validated.

The SB constructs described in this experiment had the following insert structure: Enhancer sequence-minimal promoter (YBTATA) with flanking spacers and barcode sequence-mCherry payload. The enhancer sequences of these SB constructs are shown in Table 8. The mCherry payload sequence is shown in Table 7. The YBTATA minimal promoter sequence with flanking spacers is shown in Table 10.

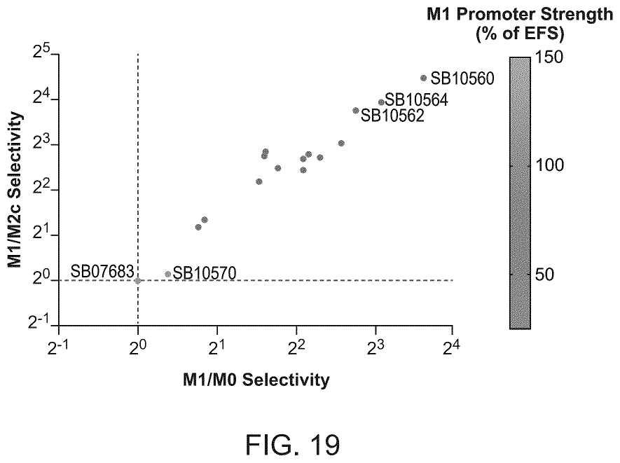

Results are shown in FIG. 19. From these 15 hits, SB10560 has the highest M1 selectivity (>10 fold) and M1 promoter strength compared to the other constructs. Additionally, SB10564 and SB10562 have >8 fold selectivity and M1 promoter strength that is ˜40% of EFS. By contrast, SB10570 is very strong (˜150% of EFS) but not very M1-state selective.

### Example 5: Master Regulator Screening in Macrophages with Constitutive Promoters

In order to identify candidate transcription factors that act as M1 master regulators, macrophages were transduced with 12 candidate M1 master regulators, then polarized to an M0, M1, or M2c state. Macrophages transduced with a GFP reporter alone were used as a negative control for phenotype changes, as GFP is expected to have no functional activity as a macrophage regulator. Macrophages transduced with a construct constitutively expressing IFN-gamma (SB09077) were used as a positive control for phenotype changes towards an M1 polarization state.

Briefly, individual lentiviral constructs were cloned/synthesized with a constitutive promoter (SFFV)expressing the candidate master regulators. On Day 0, monocytes were isolated from previously frozen primary human PBMCs using CD14+ selection via MACS. From Day 0-Day 4, CD14+ monocytes were differentiated to M0 macrophages with M-CSF. On Day 4, M0 Macrophages were passaged into ULA plates for subsequent transduction. On Day 5, M0 macrophages were transduced with lentiviral vectors containing master regulator constructs. On Day 6, the M0 macrophages were subjected to the following polarization conditions: no polarization (kept in the M0 state), M1 polarization (with IFNγ), or M2c polarization (with IL-10 and TGFβ). After 48 hrs of polarization, all cells were assessed for phenotype via flow cytometry and cytokine expression. Cell polarization phenotype was assessed via surface marker staining. All supernatants were collected for cytokine and chemokine activity measurements via Luminex.

Four surface markers were assessed via flow cytometry (CD40, CD80, CD163, and CD206) and 7 cytokine expression was measured from the supernatant (IL-10, IL-4, TNF-alpha, MIP-1beta, IFN-alpha, IL-6, IFN-gamma, GRO-alpha).

Principal component analysis was performed to assess phenotype changes in aggregate from all variables measured.

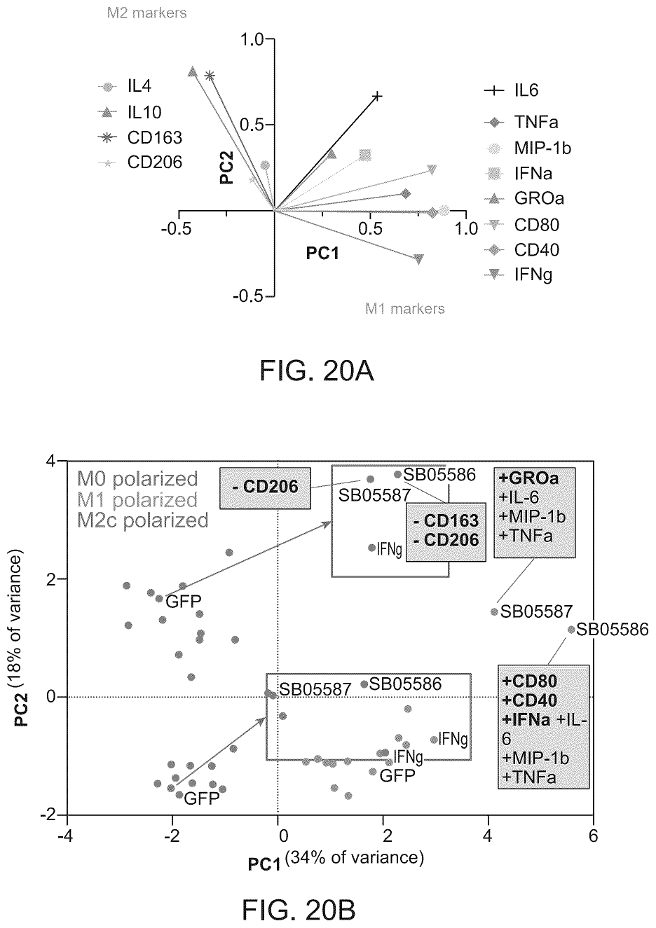

Principal component loadings for all quantified variables are depicted in FIG. 20A. Generally, all M2-associated markers cluster towards the left-hand side while all M1-associated markers cluster towards the right-hand side of the 2-dimensional principal component space. Principle component analysis of the master regulator candidates is depicted in FIG. 20B. Out of the twelve tested candidate transcription factors, only two (IRF7and p65/RelA) were identified that have activity comparable to the positive control, constitutively expressed interferon gamma. Regardless of polarization state, these master regulators drove the macrophages towards an M1-like state that are manifested by increases in M1 marker expression that is concurrent with decreases in M2 marker expression. SB05586 (construct for constitutive expression of IRF7), and SB05587 (construct for constitutive expression of p65/RelA) shifted M0 and M2c polarized macrophages to a more M1 like state (similar to IFNγ). These results indicate that IRF7 and p65/RelA can be used in a phenotype switch circuit (e.g., using a construct that operably links IRF7 or p65/RelA to an M2 state selective promoter to drive a macrophage phenotype switch from an M2 state to an M1 state), and/or in a phenotype lock circuit (e.g., using a construct that operably links IRF7 or p65/RelA to an M1 state selective promoter).

### Example 6: M1 Lock Circuit Screening

Various constructs were tested for their ability to lock macrophages into a stable M1 phenotype state. Briefly, individual lentiviral constructs were designed to express either an IFNγ payload or mCherry reporter under control of a constitutive EFS promoter or various M1 state selective promoter candidates. See Table 11.

TABLE 11
SB construct design.

| SB ID | Promoter | Regulator | Reporter | |
|---|---|---|---|---|
| SB07683 | EFS | — | mCherry | − control |
| SB09073 | | IFN-γ | — | + control |
| SB09385 | IDO1 | — | mCherry | |
| SB09406 | UBD1 | — | mCherry | |
| SB08123 | CCL19_m_25 | — | mCherry | |
| SB08125 | CCL19_m_27 | — | mCherry | |
| SB09698 | IDO1 | IFN-γ | — | |
| SB09700 | UBD1 | IFN-γ | — | |
| SB09702 | CCL19_m_25 | IFN-γ | — | |
| SB09704 | CCL19_m_27 | IFN-γ | — | |

On Day 0, monocytes were isolated from previously frozen primary human PBMCs using CD14+ selection via MACS. From Day 0-Day 4, CD14+ monocytes were differentiated to M0 macrophages with M-CSF. On Day 4, M0 Macrophages were passaged into ULA plates for subsequent transduction. On Day 7 (early AM), M0 macrophages were transduced with lentiviral vectors containing master regulator constructs in two parallel plates. On Day 7 (late PM), the two plates were processed as follows: In Plate 1, Macrophages were kept in M0 state, or polarized to M1 (with IFNγ) state, or M2c (with IL-10, and TGFβ). Polarizing to all 3 polarization states enables testing of the candidate circuits across all 3 polarization states. Leaky activity can also be detected in the states where the M1 promoter should not be turned on. In Plate 2, all cells were polarized to the M1 state with IFN-gamma.

On Day 9, all cells in Plate 1 were assessed via flow for immunophenotyping and supernatants were collected to determine initial promoter activity. For Plate 2, media was exchanged and then cells were polarized away from M1 to M0, re-polarized to M1, or transpolarized to an M2c state.

On Day 11, all cells on plate 2 were assessed via flow for immunophenotyping.

Cell polarization phenotype was assessed via surface marker staining and cytokine expression on Day=9 (Plate 1) and Day=11 (Plate 2). All supernatants were collected for cytokine and chemokine activity measurements via Luminex.

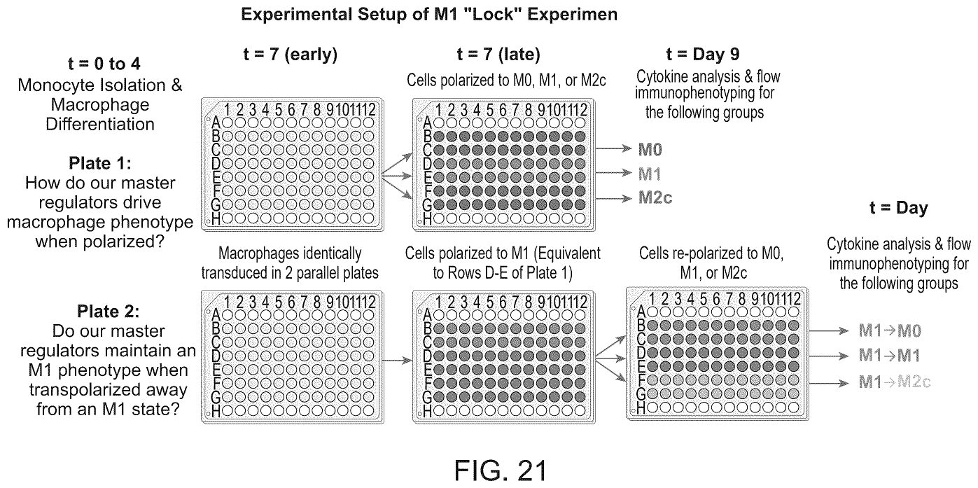

See FIG. 21 for details on the experimental timeline.

Four surface markers were assessed via flow cytometry (CD40, CD80, CD163, and CD206) and 8 cytokine expression was measured from the supernatant (IL-10, IL-4, TNF-alpha, MIP-1beta, IFN-alpha, IL-6, IFN-gamma, GRO-alpha). Principal component analysis was performed to assess phenotype changes in aggregate from all variables measured with the exception of IFN-gamma.

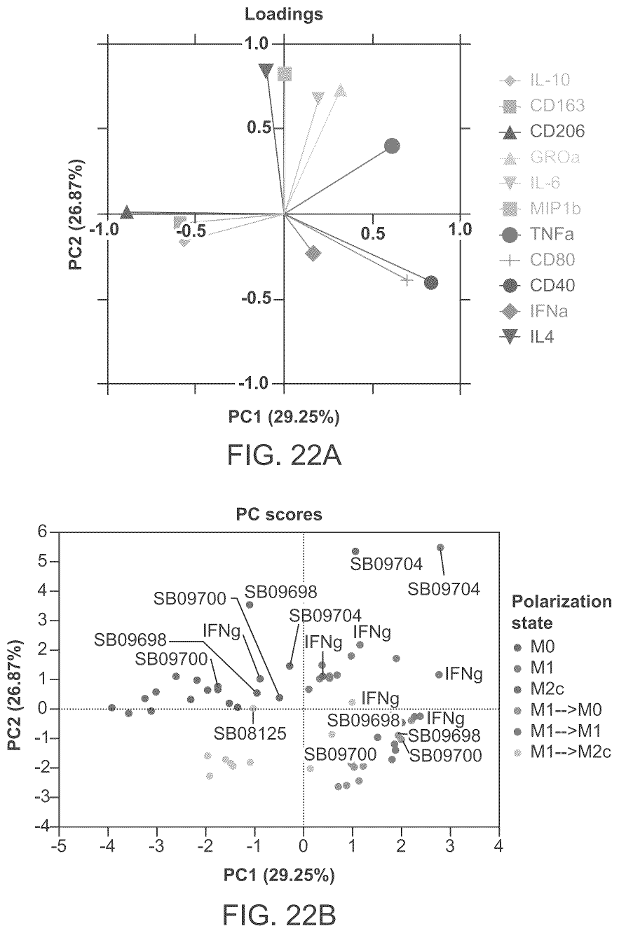

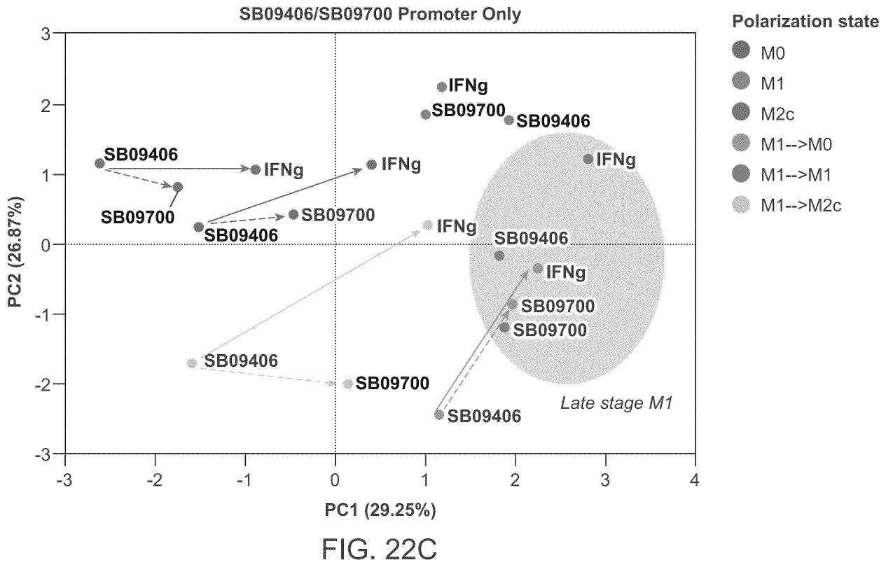

Principal component loadings for all quantified variables are depicted in FIG. 22A. Generally, all M2-associated markers (blue) clustered towards the left-hand side while all M1-associated markers (red) cluster towards the right-hand side of the 2-dimensional principal component space. Markers of macrophage intermediate state clustered towards the upper middle (yellow, orange).

FIG. 22B shows aggregated phenotypes in the 2-dimensional principal component space of all candidate M1 lock circuits across all polarization conditions. As shown, SB09698 and SB09700 resisted trans-polarization away from an M1-state (M1→M2c and M1→M0 conditions) and exhibited comparable activity to constitutively expressed IFNγ in those conditions. SB09704 & SB09698 exhibited leaky expression in the M2c state, but SB09700 did not. SB09704 exhibited large phenotype changes in pushing the cells towards the upper right quadrant with over expression of many M1-associated markers. However, SB09704 had leaky expression as demonstrated with large phenotypic shifts in both “off” states (The M0 and M2c cases).

FIG. 22C shows aggregated phenotypes in the 2-dimensional principal component space of SB09073 (positive control expressing IFNγ under EFS control), SB09700 (expressing IFN under control of M1 state-selective UBD1 promoter) and SB09406 (expressing mCherry under control of the M1 state-selective UBD1 promoter, across the different polarization conditions. Solid arrows show the phenotype shift between the positive SB09703 and the negative control SB09406, Dotted arrows show the phenotype shift between the test circuit SB09700 and the negative control SB09406. The green oval highlights the principal components space for a late-stage M1 cell phenotype.

As shown in FIG. 22C, exposure to M0, M1, or M2c polarization cues drove cells transduced with the negative control circuit towards the M0, M1, or M2c state, respectively. However, leaky activity of the SB09700 circuit was minimal (dark blue and purple dotted lines). The magenta and green data points show the ability of the SB09700 circuit to lock M1 macrophages into a stable M1 phenotype in the presence of opposing polarization cues. The cells expressing the M1-lock circuit (SB09700) were able to maintain the macrophages on the right-hand side of the 2-dimensional PCA space, suggesting maintenance of an M1-like phenotype. This is in stark contrast to its corresponding negative control (SB09406), which is unable to maintain an M1-like phenotype.

Using either screening method described above, engineered polarization-specific promoters were identified that had comparable expression levels to a constitutive promoter in the “on” state. Moreover, the “on” state activity was at least an order of magnitude higher in activity than the “off” state.

### Example 7: Assessment of Candidate M1→M2 Phenotype Switch Circuit

State-selective activity of exemplary M1 promoters for driving payload expression was assessed using flow cytometry-based measurement of fluorescent reporter expression. Six constructs were tested:

Three constructs expressing various payloads under control of the constitutive SFFV promoter: SB00006 (GFP payload), SB09075 (IL-10 payload/M2 master regulator), and SB09077 (IFN-γ payload/M1 master regulator).

Three constructs expressing various payloads under control of the CCL19 promoter (SEQ ID NO: 132): SB05116 (GFP payload), SB09079 (IL-10 payload), SB09081 (IFN-γ payload).

See Table 12, below.

| SB ID | Promoter | Cytokine Regulator | Rep. Gene |
|---|---|---|---|
| SB00006 | SFFV | | GFP |
| SB09075 | SFFV | IL-10 | |
| SB09077 | SFFV | IFN-gamma | |
| SB05116 | CCL19 | | GFP |
| SB09079 | CCL19 | IL-10 | |
| SB09081 | CCL19 | IFN-gamma | |

On Day 0, monocytes were isolated from previously frozen primary human PBMCs using CD14+ selection via MACS. From Day 0-Day 4, CD14+ monocytes were differentiated to M0 macrophages with M-CSF. On Day 4, M0 Macrophages were passaged into ULA plates for subsequent transduction. On Day 5, M0 macrophages were transduced with lentiviral vectors containing the SB constructs. Afterwards, the macrophages were subjected to the following polarization conditions: no polarization (kept in the M0 state), M2c polarization (with IL-10 and TGFβ), M1 low (with 10 ng/ml IFN-γ), M1+(with 50 ng/ml IFN-γ), and M1++ (with 50 ng/mL IFN-γ and 10 ng/ml LPS.

Activity was assessed via flow phenotyping and supernatants were collected for cytokine and chemokine measurements via Luminext at the following 3 time points after polarization: ˜14 hours, 38 hours, and 62 hours. Promoter strength and M1 Promoter polarization state selective activity were assessed according to the methods described in Example 1.

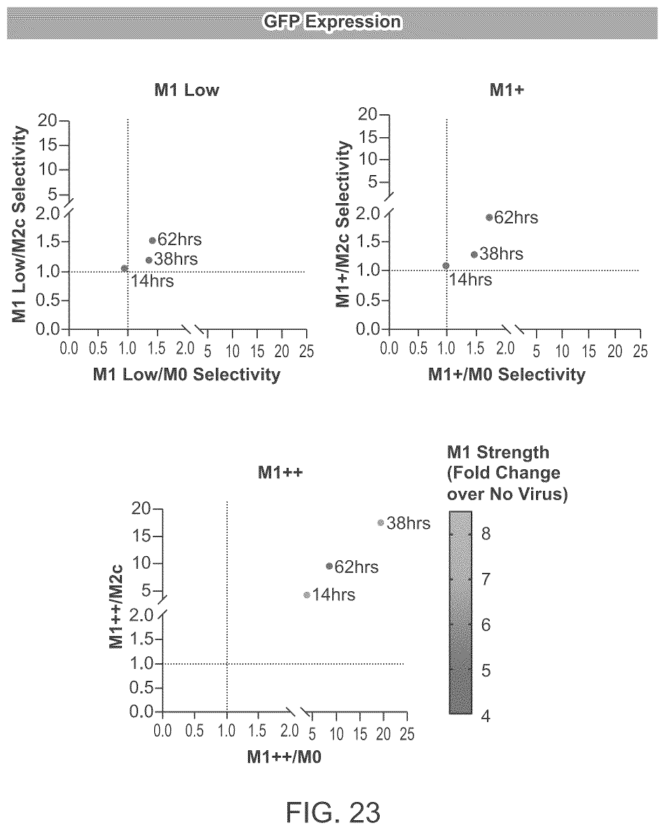

FIG. 23 depicts polarization state selective activity and promoter strength as analyzed by GFP expression. Color scale indicates the promoter strength as fold change over no virus. Results indicate optimal state-selective promoter activity and strength with strong (M1++) polarization cues at all tested time points, with the strongest activity and highest selectivity at ˜38 hours post-polarization.

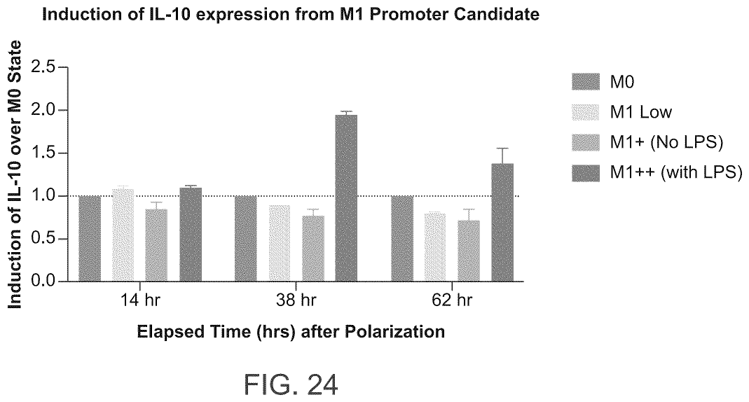

FIG. 24 depicts IL-10 expression induced by the tested M1 polarization conditions as compared to the M0 polarization condition. The M1-promoter candidate CCL19 promoter was paired with an M2 cytokine (IL-10) to drive inducible IL-10 as an M2 master regulator in a phenotype switch circuit. IL-10 expression was measured from the supernatant of the cells. Expression was normalized to the M0 state to determine level of IL-10 induction. As shown, polarization with the M1++ polarization cues induced M1-state selective IL-10 induction over the M0 state, at 38 and 62 hours post-induction.

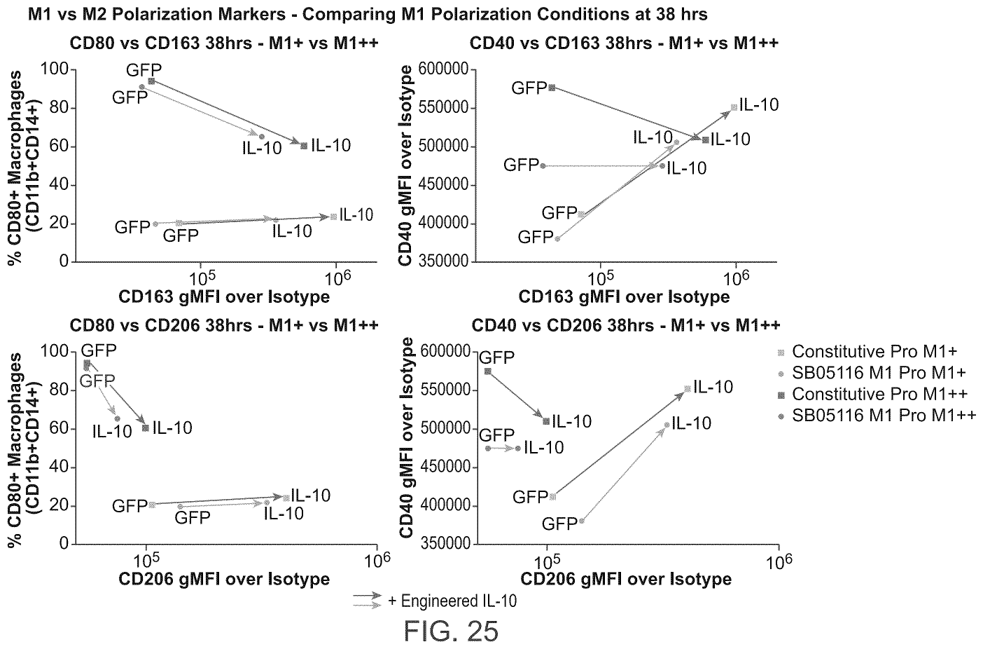

In addition, cell surface markers were used to assess changes in cell phenotype due to phenotype switch circuit activity (FIG. 25). M2-associated surface markers (CD163, and CD206) are shown on the x-axes while M1-associated surface markers (CD80 and CD40) are shown on the y-axes. Promoter constructs driving GFP expression were used as negative controls for the phenotype changes, as GFP is expected to have no functional activity as a macrophage regulator. SB09075 (expressing the M2 master regulator IL-10 under control of the constitutive SFFV promoter) was used as a positive control for phenotype changes towards an M2 polarization state. All data with constitutive promoters are depicted as squares, whereas all data with M1-state specific promoter (promoter sequence from SB05116) are depicted as circles. All data are labeled with the respective payloads. At 38 hrs, with M1 polarization, the M1-state specific promoter driving IL-10 expression was able to shift the macrophages towards an M2-like phenotype that is comparable to the constitutive control.

### Example 8: M2 Promoter Screening

Total RNA-Seq was performed in-house to identify protein coding genes and non-coding RNA that were differentially up-regulated in the M2 states. 48 native promoter sequences for those differentially up-regulated genes were designed into reporter constructs for screening M2-state selective activity according to methods described in Example 1, except that for mCherry reporter constructs, mCherry instead of GFP was used as the signal readout for determining state-selective activity and promoter strength calculations. A constitutive promoter control (EFS) was screened in parallel to determine strong, non-selective promoter activity.

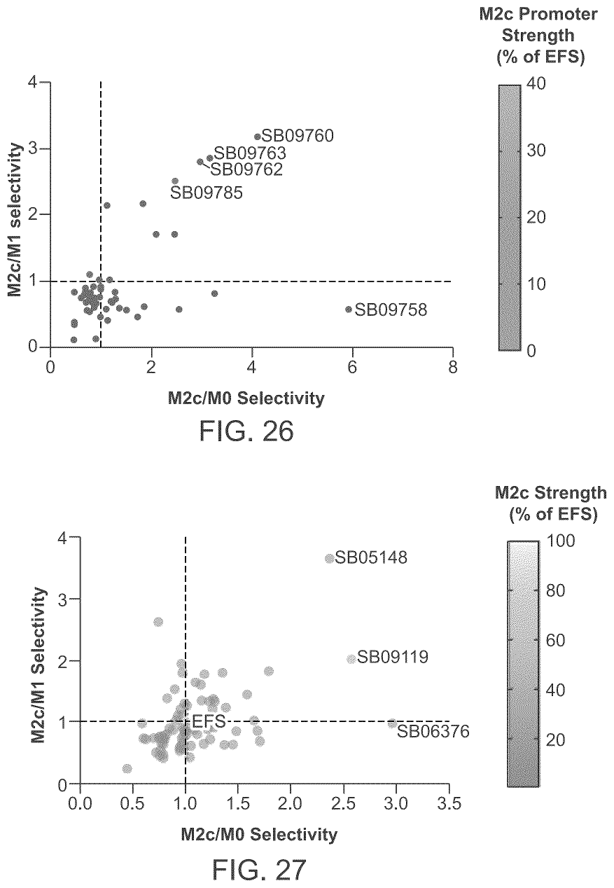

Results are shown in FIG. 26. Dotted lines represent non state-selective promoter activity. Promoter strength was normalized to constitutive promoter (% of EFS).

Five promising M2-state selective promoters were identified (SB09758, SB09760, SB09762, SB09763, SB09785). Of all candidate promoters screened, SB09760, SB09762, SB09763, SB09785 have the highest M2 selectivity over both the M0 and M1 states (>2 fold). SB09758 exhibited the highest M2c/M0 selectivity (˜6 fold). SB09762 and SB09785 exhibited the highest M2c promoter strengths (18% and 38% of EFS, respectively).

Sequences of the M2-state selective promoters designed into the SB09758, SB09760, SB09762, SB09763, and SB09785 constructs are shown in Table 1.

### Example 9: M2 Promoter Screening

Differentially up-regulated genes in the M2 state were identified from multiple literature studies and in-house RNA-Seq data. An additional 75 native promoter sequences for those differentially up-regulated genes were designed into reporter constructs for screening M2-state selective activity according to methods described in Example 1. A constitutive promoter control (EFS) was screened in parallel to determine strong, non-selective promoter activity.

Results are shown in FIG. 27. Dotted lines represent non state-selective promoter activity. Promoter strength was normalized to constitutive promoter (% of EFS).

Three additional M2-state selective promoters were identified. SB05148, SB09119, and SB06376 have promising M2c state-selective promoter activity. SB06376 exhibited M2c selective activity over the M0 state, while SB05148 and SB09119 exhibited M2c selective activity over both M1 and M0 states.

### Example 10: M2 Promoters Engineered from ATAC-Seq Nominated Enhancers

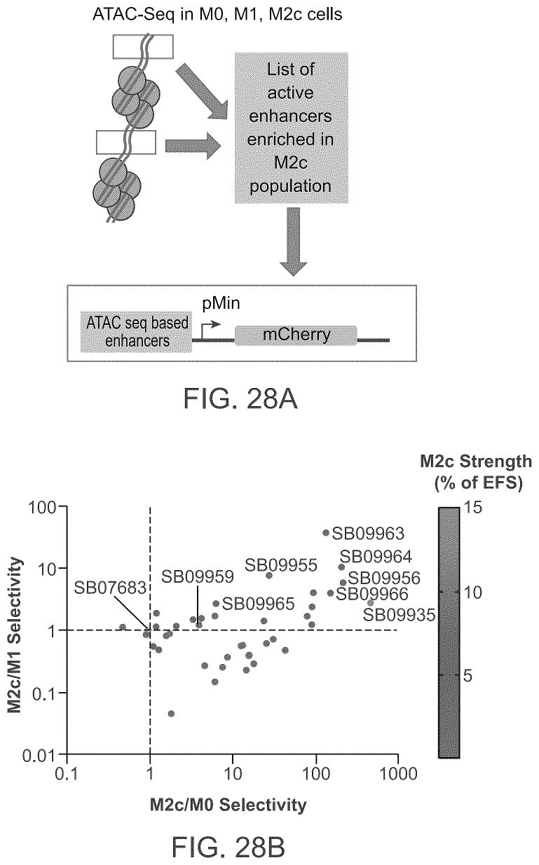

ATAC-Seq was performed on M0, M1, and M2c polarized macrophages to identify regions of differentially open chromatin in each polarization state. Regions of putative enhancers specific for each polarization state were bioinformatically selected from the ATAC-Seq data. 40 putative enhancers were then combined with a minimal promoter to create candidate M2-state selective promoter. See FIG. 28A. A constitutive promoter control (EFS) was screened in parallel to determine strong, non-selective promoter activity.

The SB constructs described in this experiment had the following insert structure: Enhancer sequence-minimal promoter (YBTATA) with flanking spacers and barcode sequence-mCherry payload. The enhancer sequences of these SB constructs are shown in Table 8. The mCherry payload sequence is shown in Table 7. The YBTATA minimal promoter sequence with flanking spacers is shown in Table 10.

Results are shown in FIG. 28B. Dotted lines represent non state-selective promoter activity. Promoter strength was normalized to constitutive promoter (% of EFS). As shown, 8 promising M2-state selective promoters were identified (SB09959, SB09965, SB09955, SB09963, SB09964, SB09956, SB09966, and SB09935). These promoters exhibited high selectivity for the M2 state (>10-100-fold) with no measurable activity in the M0 or M1 states. These promoters exhibited <15% of the strength of the EFS promoter.

### Example 11: M2 Promoters Nominated from MPRA Library Screening

Three synthetic promoter libraries were designed and screened for promoter activity via a high-throughput MPRA-based assay, according to the methods described in Example 1. Each promoter library contained >10,000 rationally-designed synthetic promoters (>35,000 constructs total). One library included all known human transcription factor binding sites, whereas the remaining two libraries were designed with motifs targeted to be active in either the M1 or M2c state. From the MPRA-assay, >75 hits from all 3 libraries were selected from sequencing results to have higher activity in the M2c state and were then clonally validated.

The SB constructs described in this experiment, with the exception of SB09846, had the following insert structure: Enhancer sequence-minimal promoter (YBTATA) with flanking spacers and barcode sequence-mCherry payload. SB09846 had the following structure: Enhancer sequence-minimal promoter (minCMV) with flanking spacers and barcode sequence-mCherry payload. The enhancer sequences of these SB constructs are shown in Table 8. The mCherry payload sequence is shown in Table 7. The YBTATA minimal promoter sequence with flanking spacers is shown in Table 9.

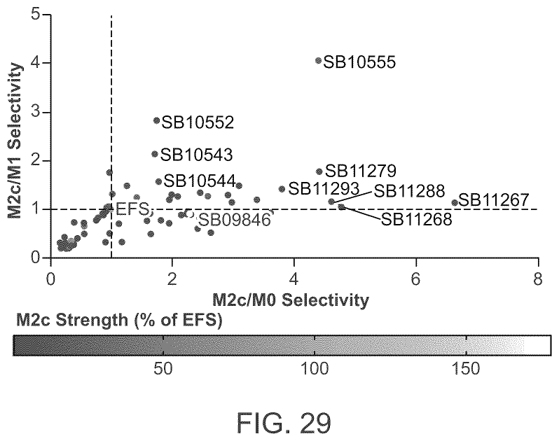

Results are shown in FIG. 29. Dotted lines represent non state-selective promoter activity. Promoter strength was normalized to constitutive promoter (% of EFS).

From the universal transcription factor library 1 hit (SB09846) exhibited very strong promoter activity (>170% of EFS) and >2-fold M2c selectivity over the M0 state. From the M2-targeted library, 4 hits exhibited M2c-selective activity over the M1 state (SB10543, SB010544, SB10552, SB010555). From the M1-targeted library, 5 hits exhibited high M2c selective activity over the M0 state, but not the M1-state (SB11267, SB11268, SB11279, SB11288, SB11293). SB10545 and SB11297 were the strongest clonally validated M2 promoters from the M2 & M1-targeted libraries, respectively (data not shown) with ˜25-30% of the strength of EFS.

### Example 12: Re-Engineering of Selected M2 Promoters from ATAC-Seq Nominated Enhancers

Eight M2-state selective promoter constructs identified in Example 10 (SB09959, SB09965, SB09955, SB09963, SB09964, SB09956, SB09966, and SB09935) were found to be highly selective but had lower promoter strength as compared to EFS. Accordingly, these promoters selected for re-engineering (see FIG. 30, left panel, pink circles) by combining the enhancer regions from these eight constructs with a variety of minimal or core promoter sequences. The-re-engineering yielded promoters with improved strength and selectivity over the M0 and/or M1 state, as compared to the original promoters (see FIG. 30, right panel, green oval).

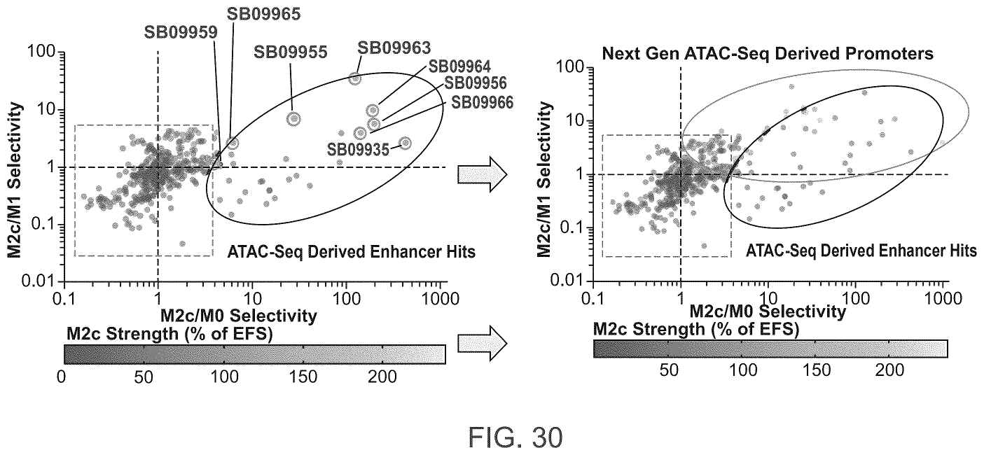

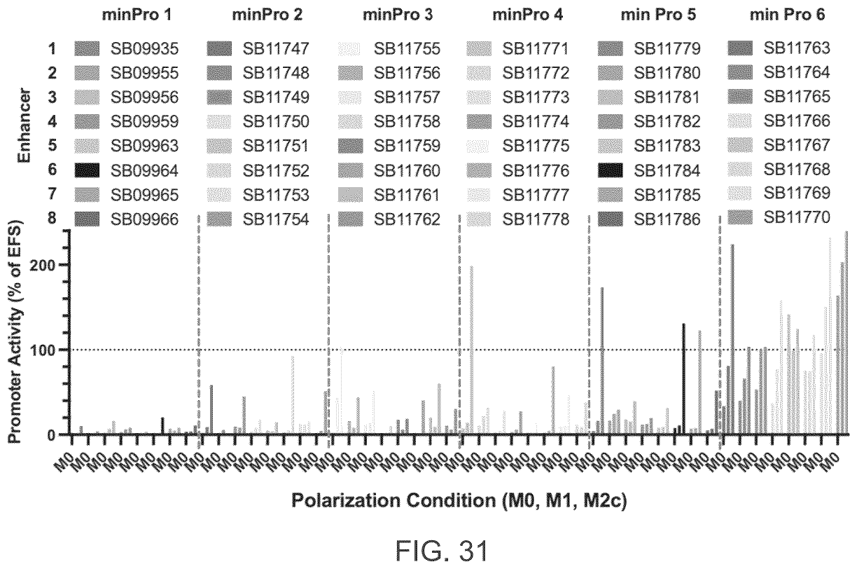

FIG. 31 depicts the promoter constructs tested (top panel) and promoter activity across different macrophage polarization conditions.

The 8 putative enhancers selected from earlier screening were paired with 5 minimal or core promoters (6 total including the original, minPro 1). In the bottom panel of FIG. 31, each group of 3 of the same colored-bars represent a single enhancer and minimal promoter pairing that was tested in the M0, M1, and M2c polarization states, respectively (while the X axis of FIG. 31 shows only M0 labeling, promoter activity of each construct is depicted as three lines of the same color, representing activity in the M0, M1, and M2c polarization states, from left to right). Candidate promoter activity was assessed via reporter expression normalized to expression of a strong, constitutive promoter (EFS). minPro1, 2 and 3 correspond to a consensus YBTATA promoter, a miniaturized CMV promoter (minCMV), and a minimal TK promoter (minTK), respectively. MinPro4 is the SCP3 promoter sequence, and minPro 5 is a hybrid sequence between the YBTATA sequence and the SCP3 sequence. MinPro6 is a fully synthetic, compact promoter hit that was derived from SPECS library screening (SB09846). MinPro 6 is an array of five of the same ELF1 transcription factor binding sites repeated in tandem linked to a minCMV promoter. Blue dashed lines divide up the enhancer-based promoter candidates by each minimal/core promoter type. Promoter strength and macrophage state-selective activity was assessed according to methods described in Example 1. Tested core promoters improved the dynamic range of promoter strength over the original minimal promoter used, YBTATA (minPro1). For many promoter candidates, M2c-state selective promoter strength was increased to >100% of EFS for many promoter candidates.

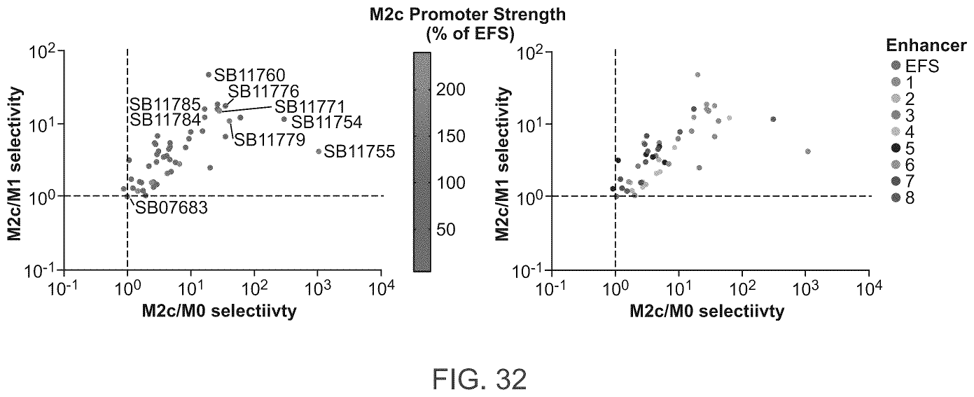

FIG. 32 depicts M2 state selective activity over the M1 and M0 states, respectively. Promoter strength was normalized to a strong, constitutive EFS promoter control (SB07683) that has no state-selective activity. The top 8 strongest and most M2c selective promoters were identified to be SB11754 (Enhancer 8—minPro2), SB11755 (Enhancer 1—minPro3), SB11760 (Enhancer 6—minPro3), SB11771 (Enhancer 1—minPro4), SB11776 (Enhancer 6—minPro4), SB11779 (Enhancer 1—minPro5), SB11784 (Enhancer 6—minPro5), and SB11785 (Enhancer 7—minPro5). See FIG. 32, Left panel. All of these promoters exhibited at least 10-fold selective activity in the M2c state over either the M0 or M1 states (left panel). Other enhancer-minimal promoter combinations that exhibited at least 10-fold selective activity in the M2c state over M0 or M1 states include: SB11758 (Enhancer 4—minPro3), SB09964 (Enhancer 6—minPro1) and SB11752 (Enhancer 6—minPro2). When grouping selective activity by the same enhancer elements (right panel), enhancer elements 1 (red dots) and 6 (brown dots) were the most robust enhancers that were selective across nearly all minimal and core promoters. See FIG. 32, right panel. As indicated in FIG. 32, constructs clustered more closely by their enhancer than by their core promoters suggesting that enhancers were the main drivers of selectivity.

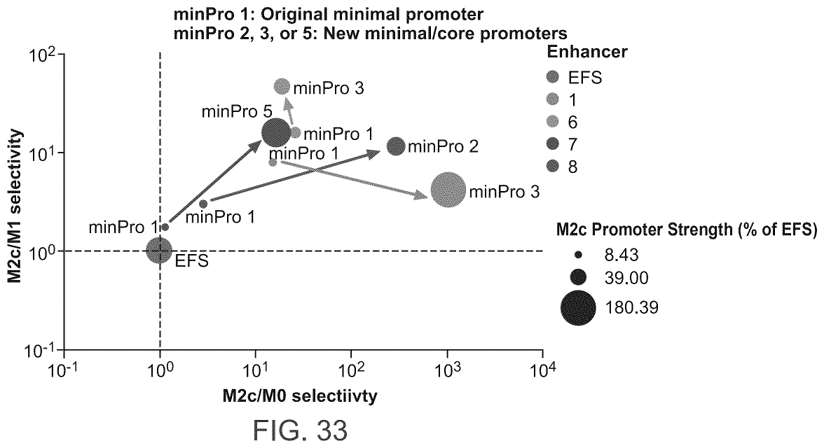

FIG. 33 plots the “original” promoter design (with YBTATA as the minimal promoter) and best next generation promoter design in a pair-wise fashion.

The color, size, and text of each data point represents the enhancer, the promoter strength, and the minimal/core promoter, respectively. Five of the eight enhancers tested (enhancer 1, 6, 7 and 8) had improved M2c-selective activity and promoter strength with tested core promoters. The pairing of the enhancer elements with different minimal or core promoters improved M2c selectivity over the M1 and/or M0 states, and boosted overall promoter strength in unpredictable ways.

### Example 13: Re-Engineering of M1 State-Selective Promoters Derived from Native IDO1 Promoter

The native IDO1 promoter sequence was rationally re-engineered into 3^{rd} generation promoter candidates based on the results of the promoter ablation screening results described in Example 2.

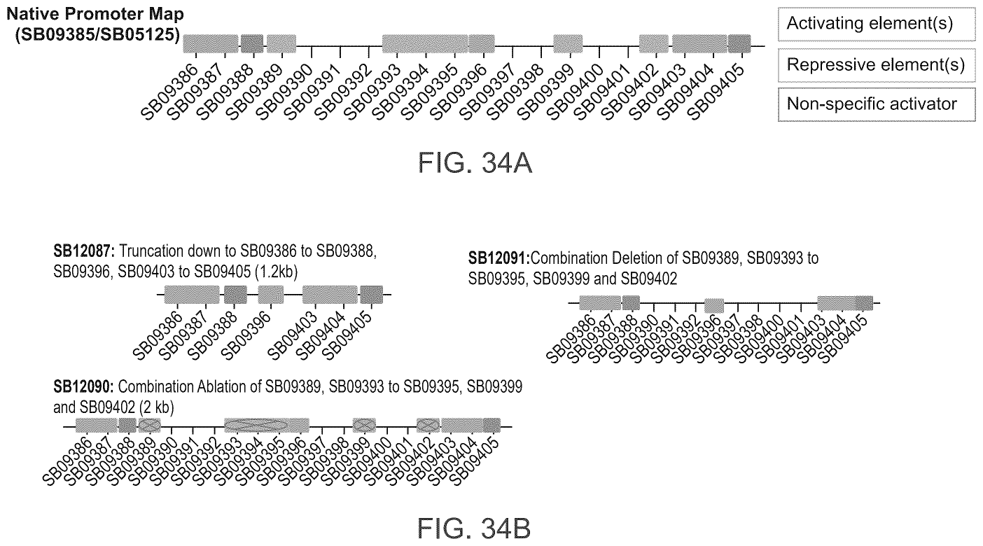

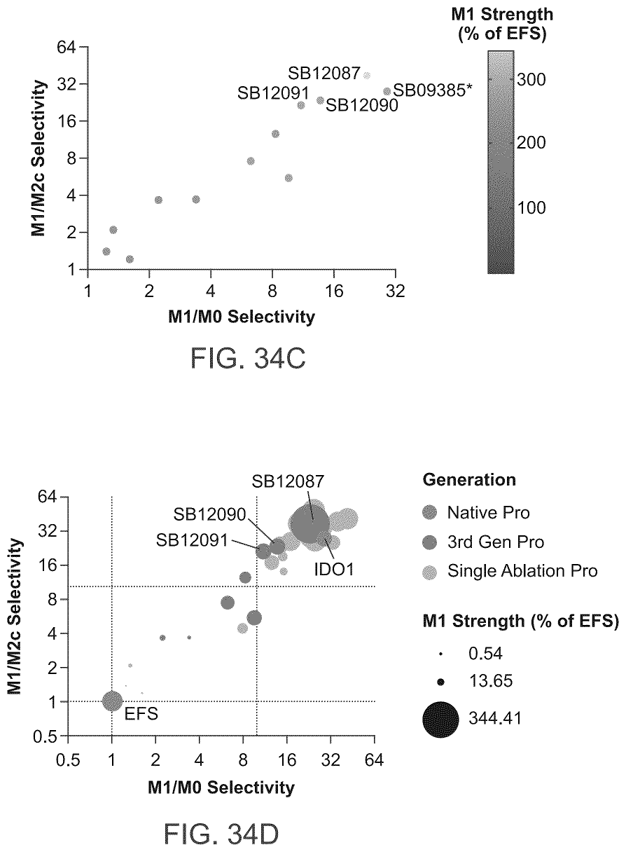

FIG. 34A depicts the functional regions of the IDO1 native promoter sequence that were mapped from the ablation screening experiment of Example 2.

Re-engineering included, e.g., truncation of the native promoter down to neighboring M1 selective activating elements, tandem repeats thereof, and ablation or deletion of one or more M1-selective repressive or leaky elements. As used in this example, ablation refers to replacement of the nucleotide motif with an inert sequence, while deletion refers to deletion of the motif without replacement. Any of these regulatory elements could be truncated, deleted, ablated, repeated and/or re-combined into a 3^{rd} generation promoter design. Twelve such constructs were made and screened for M1 state-selective promoter activity and promoter strength using methods described in Example 1, as compared to the native promoter construct SB09385.

The designs of the top three 3^{rd} generation promoter designs (constructs SB12087, SB12090, and SB12091) are shown in FIG. 34B.

Results are shown in FIG. 34C and FIG. 34D. FIG. 34D shows an overlay of all promoter derivatives of the native IDO1 promoter in one plot. The color of each data point represent the promoter type. The size of each data point represents the promoter strength in the M1 state. Purple data points represent the native promoter (IDO1) and the constitutive promoter control (EFS). Orange data points represent the data for all single ablation promoter variants, whereas red data points represent rationally re-engineered 3^{rd} generation variants. Among the tested 3^{rd} generation promoters, SB12087, SB12090, and SB12091 exhibited high M1 state selective activity over M2c or M0 states and improved or comparable promoter strength to the native IDO1 promoter. In particular, SB12087 exhibited improved M1 promoter strength over all ablation variants (>340% of EFS) while still maintaining high M1-state selective activity comparable to the native promoter and single ablation promoters.

### Example 14: Re-Engineering of M1 State-Selective Promoters Derived from Native UBD1 Promoter

The native UBD1 promoter sequence was rationally re-engineered into 3^{rd} generation promoter candidates based on the results of the promoter ablation screening results described in Example 3.

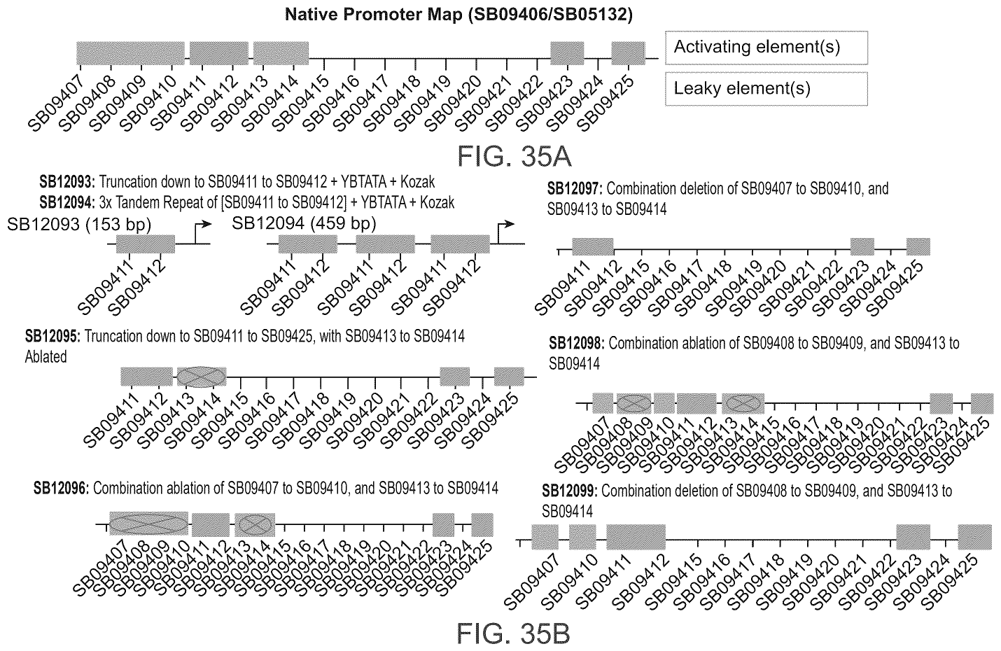

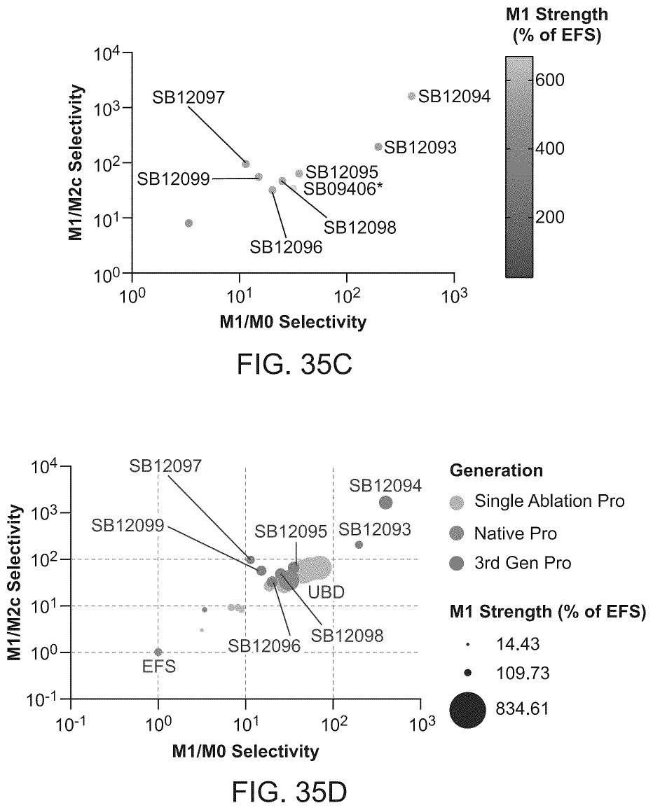

FIG. 35A depicts the functional regions of the UBD1 native promoter sequence that were mapped from the ablation screening experiment of Example 3.

Re-engineering included, e.g., truncation of the native promoter down to neighboring M1 selective activating elements, tandem repeats thereof, and deletion of one or more M1-selective repressive or leaky elements. As used in this example, ablation refers to replacement of the nucleotide motif with an inert sequence, while deletion refers to deletion of the motif without replacement. Any of these regulatory elements could be truncated, deleted, ablated, repeated and/or re-combined into a 3^{rd} generation promoter design. Eight such constructs were made and screened for M1 state-selective promoter activity and promoter strength using methods described in Example 1, as compared to the native promoter construct SB09406. The designs of select 3^{rd} generation promoters (constructs SB12093-12099) are shown in FIG. 35B.

Results are shown in FIG. 35C and FIG. 35D. FIG. 35D shows an overlay of all promoter derivatives of the native UBD1 promoter in one plot. The color of each data point represent the promoter type. The size of each data point represents the promoter strength in the M1 state. Purple data points represent the native promoter (UBD1) and the constitutive promoter control (EFS). Orange data points represent the data for all single ablation promoter variants, whereas red data points represent rationally re-engineered 3^{rd} generation variants. Among the tested 3^{rd} generation promoters, SB12093-SB12099 exhibited high M1 state selective activity over M2c or M0 states and promoter strength comparable or higher than EFS. In particular, SB12093 and 12904 exhibited >10-fold higher M1-state selectivity over the native UBD1 promoter and single ablation variants.

### Example 15: Testing of Membrane-Tethered IFN-γ in Macrophages with Constitutive or Selected M1 State-Selective Promoters

In the context of macrophage polarization lock and switch circuits, membrane tethered cytokines can beneficially provide signaling inputs to induce the desired polarization state in an autocrine fashion, at reduced risk of diffusing away from the producer cell and inducing activity in off target cells in vivo.

Membrane-tethered IFN-γ was engineered to comprise a truncated human IFN-γ sequence (e.g., having the amino acid sequence SEQ ID NO: 477) operably linked (e.g., via a linker having the amino acid sequence SEQ ID NO: 479) to a transmembrane domain (e.g., a transmembrane domain of the B7-1 protein, e.g., having the amino acid sequence SEQ ID NO: 481).

Individual lentiviral constructs were designed to either express soluble IFN-γ or membrane-tethered IFN-γ under control of EFS promoter or selected M1 promoter candidates. The construct SB07683 (EFS-mCherry) was used as a positive control for expression and a negative control for phenotype lock. The construct SB11454 (EFS-soluble IFN-γ) was used as a positive control for M1 phenotype lock. See Table 13, below.

TABLE 13
SB construct design

| SB ID | Promoter Regulator | Reporter | |
|---|---|---|---|
| SB07683 | EFS | mCherry | — |
| SB11454 | EFS | Soluble IFNg | |
| SB11455 to SB11463 | Select M1 Promoter | Soluble IFNg | mCherry |
| | Candidates | | |
| SB11497 | EFS | Tethered IFNg | mcherry |
| SB11498 to SB11506 | Select M1 Promoter | Tethered IFNg | mcherry |
| | Candidates | | |

Briefly, M0 macrophages were polarized on day 7 (following lentiviral transduction on the same day) and processed as described in Example 6 (see FIG. 21).

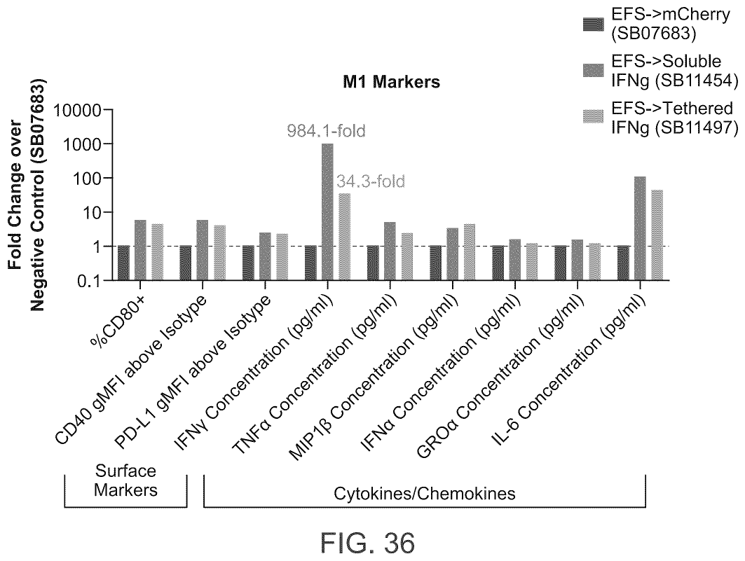

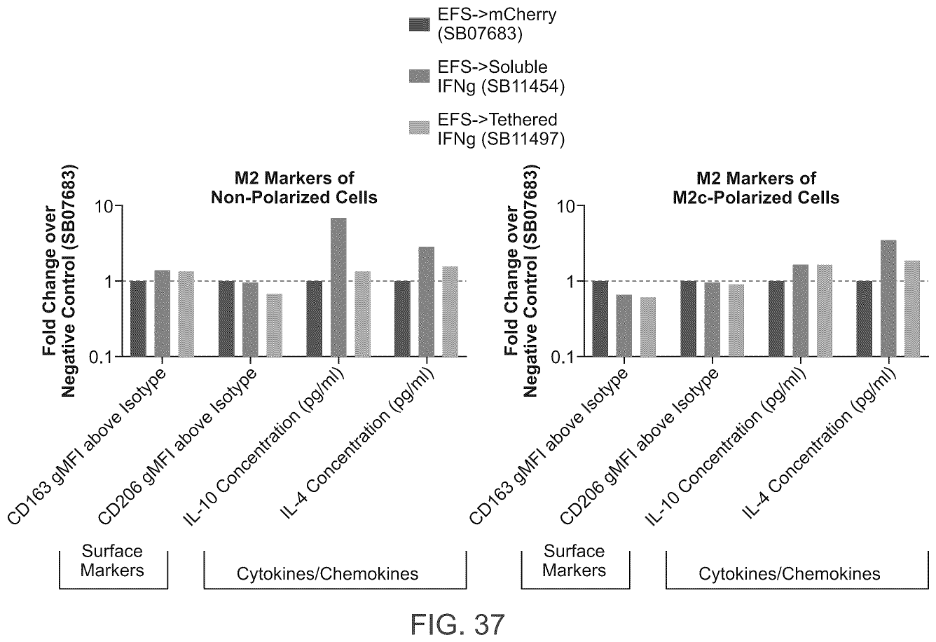

FIGS. 36 and 37 depict results for EFS constructs (expressing mCherry, IFN-γ payload, tethered IFN-γ).

FIG. 36 depicts expression of M1 cytokines and surface markers in M0 cells (Plate 1, Day 9). Without wishing to be bound by theory, payloads that act as M1 master regulators should be able to boost expression of M1 markers. To assess M1 master regulator activity, 9 M1-associated markers were assessed compared to a negative control. These included expression of 3 cell surface markers measured from flow cytometry, and 6 cytokines or chemokines measured from Luminex. Constitutively expressed soluble IFN-γ increased expression of M1 surface markers and cytokines in the M0 cells. The tethered IFN-γ payload also increased M1-associated markers over negative control. This suggests that the membrane tethered IFN-γ payload can act as a master regulator to drive an M1-like state in macrophages. As expected, IFN-γ levels were much higher in the supernatant for cells expressing soluble IFN-γ than tethered IFN-7.

FIG. 37 depicts expression of M2 cytokines and surface markers of M0 and M2c polarized cells (Plate 1, Day 9).

Without wishing to be bound by theory, payloads that act as M1 master regulators should be able to mitigate the expression of M2-associated markers. To assess M1 master regulator activity, 4 M2-associated markers were assessed compared to a negative control. These included expression of 2 cell surface markers measured from flow cytometry, and 2 cytokines measured from Luminex. In both the M0 and M2c polarization states, tethered IFNg induced lower levels of M2 associated markers and cytokines compared to soluble IFNg. The results of FIGS. 36 and 37 taken together results suggest that membrane-tethered IFN-γ can drive macrophages toward an M1-like and away from an M2-like state, as compared to soluble IFN-7.

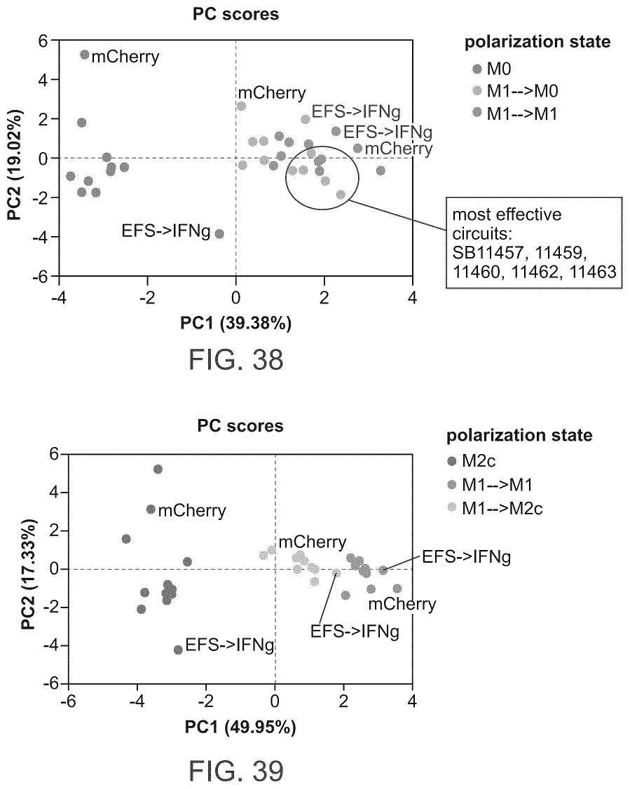

FIGS. 38 and 39 depict results for M1 lock circuits expressing soluble IFN-γ payload from candidate M1 state-selective promoters.

FIG. 38 depicts results from an experiment assessing repolarization to an M0 state (Plate 2, Day 11). Macrophages were transduced with candidate M1 lock circuits expressing soluble interferon gamma, then polarized according to experimental timeline as described previously (polarization to M1 on day 7 followed by repolarization to M0 on day 9). As a negative control for phenotype changes, the macrophages were transduced with an mCherry reporter, which has no functional activity. As a positive control for phenotype changes towards an M1 polarization state, the macrophages were also transduced with a construct constitutively expressing IFN-gamma. After 48 hrs of polarization (day 9), all cells on plate 1 were assessed for phenotype via flow cytometry and cytokine expression. After an additional 48 hrs thereafter (day 11), the cells on plate 2 (only previously polarized to an M1 state and subsequently polarized to M0, M1, or M2c on day 9) were also assessed for phenotype.

Five surface markers were assessed via flow cytometry (CD40, CD80, CD163, and CD206, PDL1). Cytokine levels were measured from the supernatant (IL-10, IL-4, TNF-alpha, MIP-1beta, IFN-alpha, IL-6, IFN-gamma, GRO-alpha). Principal component analysis was performed to assess phenotype changes in aggregate from all variables measured. Aggregated phenotypes in the 2-dimensional principal component space of all candidate M1 lock circuits for three polarization conditions (the M0 “basal” condition, the M1→M0 “repolarized” condition, and M1→M1 “target” condition) are plotted in FIG. 38. The constructs SB11457, 11459, 11460, 11462, 11463 in the M1→M0 condition have shifted closer to the PCA space occupied by the M1→M1 condition, as compared to the mCherry control. By contrast, these constructs in the M0 condition remain clustered near the mCherry control, and away from EFS:IFNg construct, in the M0 state. These results indicate that the indicated circuits can resist repolarization from M1→M0 (phenotype locking the cells in the M1 state) without inducing a large shift towards the M1 state in the basal M0 state. The most effective M1 soluble IFN-γ lock circuits for maintaining an M1-like state in the presence of an M1→M0 depolarizing cue included SB11457, SB11459, SB11460, SB11462, SB11463.

Similarly, the activity for all of the Select M1 Promoter Candidate constructs in Table 13 were plotted for 3 different polarization conditions (the M2c “polarized” condition, the M1→M2c “trans-polarized” condition, and M1→M1 “target” condition). See FIG. 39. Many of the soluble IFN-γ M1 lock circuits subjected to M1→M2 transpolarization exhibited a mild shift towards the M1→M1 state relative to mCherry; however, the shift was less than EFS:IFNg.

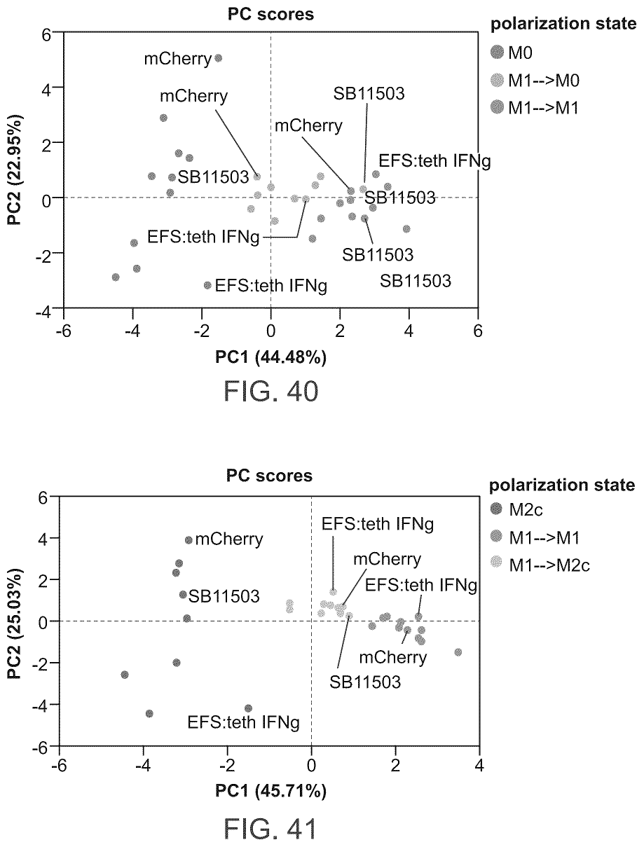

FIGS. 40 and 41 depicts results for candidate M1 lock circuits expressing the membrane-tethered IFN-γ payload.

Macrophages were transduced with candidate M1 lock circuits expressing tethered interferon gamma, then polarized according to experimental timeline as described previously. As a negative control for phenotype changes, the macrophages were transduced with an mCherry reporter, which has no functional activity. As a positive control for phenotype changes towards an M1 polarization state, the macrophages were also also transduced with a construct constitutively expressing IFN-gamma. After 48 hrs of polarization, all cells on plate 1 were assessed for phenotype via flow cytometry and cytokine expression. After an additional 48 hrs thereafter, the cells on plate 2 (only previously polarized to an M1 state) were also assessed for phenotype. Five surface markers were assessed via flow cytometry (CD40, CD80, CD163, and CD206, PDL1) and 8 cytokine expression was measured from the supernatant (IL-10, IL-4, TNF-alpha, MIP-1beta, IFN-alpha, IL-6, IFN-gamma, GRO-alpha). Principal component analysis was performed to assess phenotype changes in aggregate from all variables measured. Aggregated phenotypes in the 2-dimensional principal component space of all candidate M1 lock circuits for three polarization conditions (the M0 “basal” condition, the M1→M0 “re-polarized” condition, and M1→M1 “target” condition) are plotted in the FIG. 40. SB11503 circuit in the M1→M0 state shifts towards the M1→M1 state with low leakiness (it stays clustered with the rest of the circuits in the M0 state). FIG. 41 depicts activity of the same constructs across 3 polarization conditions (the M2c “polarized” condition, the M1→M2c “trans-polarized” condition, and M1→M1 “target” condition). As depicted in FIG. 41, SB11503 in the M1→M2c condition was closest to the M1 M1 cluster, but shifts were small and the two groups were close together in the PCA space.

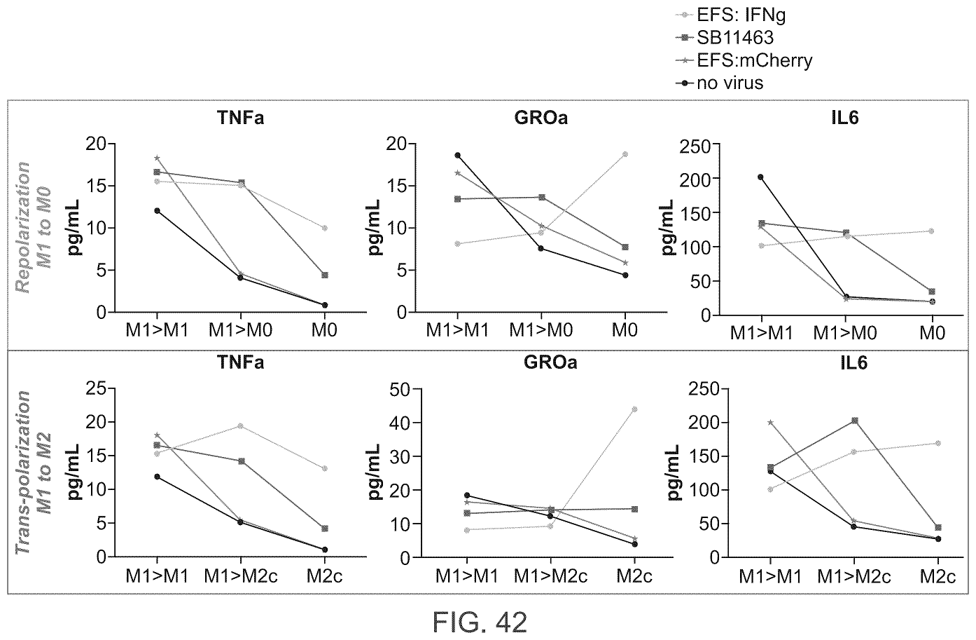

FIG. 42 depicts performance of SB11463 as assessed using TNFalpha, GROalpha and IL-6 markers. From the phenotype lock circuit screening, TNFalpha, GROalpha and IL-6 were selected as a dynamic markers that were correlative with polarization, re-polarization, and trans-polarization. For all three of these markers, in the absence of a functional payload (EFS-mCherry and no virus conditions; red stars and black circles, respectively), higher M1 cytokine expression was generally observed in the polarized (M1→M1) state with lower activity in the M2c, M0 state, re-polarized (M1→M0) or transpolarized (M1→M2c) states. With a constitutive functional payload like soluble IFNg (EFS: IFNg; gray circles), the cells generally maintained high expression of these M1-associated markers suggesting that the circuit can lock the cells in an M1-like state. The constitutive promoter expressing soluble IFNg served as a positive control with the maximum possible activity we can expect to observe in the M1 lock circuit. Across most markers, SB11463 (purple squares) exhibited maintenance of M1 markers in the repolarized or transpolarized (M1→M0 or M1→M2c) state with minimal expression in the basal (M0 or M2c) state.

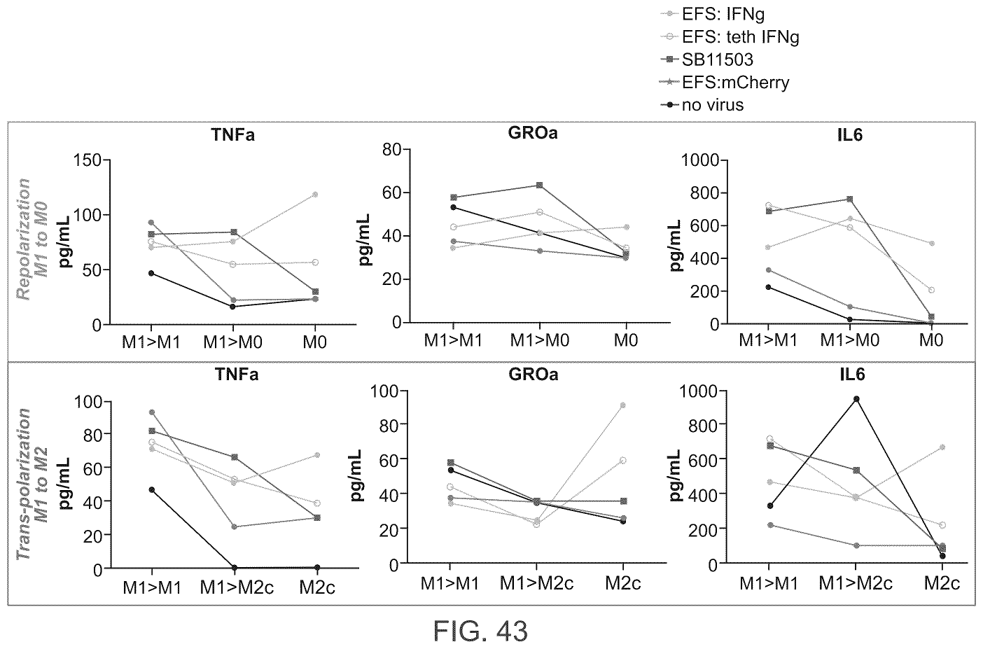

FIG. 43 depicts performance of SB11503 as assessed using TNFalpha, GROalpha and IL-6 markers. For all three of these markers, in the absence of a functional payload (EFS-mCherry and no virus conditions; red stars and black circles, respectively), higher M1 cytokine expression was generally observed in the polarized (M1→M1) state with lower activity in the M2c, M0 state, re-polarized (M1→M0) or transpolarized (M1→M2c) states. With a constituitive functional payload like soluble IFNg (EFS: IFNg; gray circles), the cells generally maintained high expression of these M1-associated markers suggesting that the circuit can lock the cells in an M1-like state. The constitutive promoter expressing soluble IFNg served as a positive control with the maximum possible activity we can expect to observe in the M1 lock circuit. Across most markers, SB11503 exhibited maintenance of M1 markers in the repolarized (M1→M0) or transpolarized (M1→M2c) state with minimal expression in the basal (M0 or M2c) state.

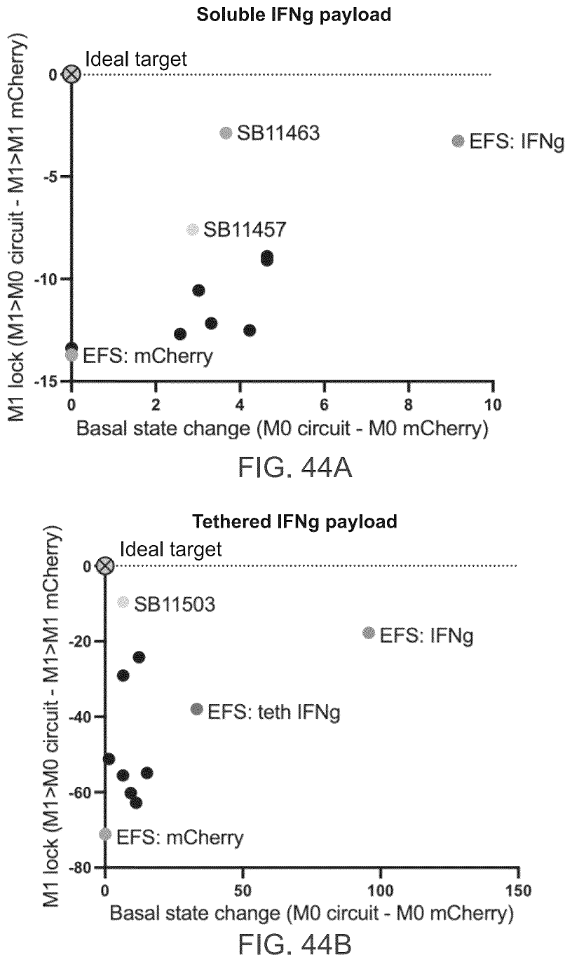

FIGS. 44A and 44B depict TNFalpha output in the M1→M0 state plotted against TNFalpha output in the M0 state, for the candidate M1-soluble IFN-γ circuits (FIG. 44A) and the candidate M1-tethered IFN-γ circuits (FIG. 44B). TNFa is shown in greater detail in these figures because it was the most dynamic marker of repolarization and transpolarization for the mCherry negative control, indicating the most opportunity for assessing impact of a phenolock circuit. The x-axis was calculated by subtracting the TNFa production (as measured by Luminex) in the M0 state of the negative control circuit (EFS:mCherry) from the M0 TNFa production of the circuit of interest. The y-axis was calculated by subtracting the TNFa production in the late M1 time point (M1 to M1 polarization state) of the negative control circuit (EFS:mCherry) from the TNFa production in the repolarized (M1→M0) state of the circuit of interest. The x-axis therefore represents the change in TNFa caused by the circuit in the basal state (ideal circuits will keep this number low). The y-axis represents the change in TNFa caused by the circuit in the repolarized state relative to the M1 state. The higher the y-value, the greater lock capability the circuit has. When plotted this way, we see that SB11463 and SB11503 exhibited an optimal combination of M1 phenotype locking ability with low basal activation for the soluble IFNg and tethered IFNg payloads, respectively.

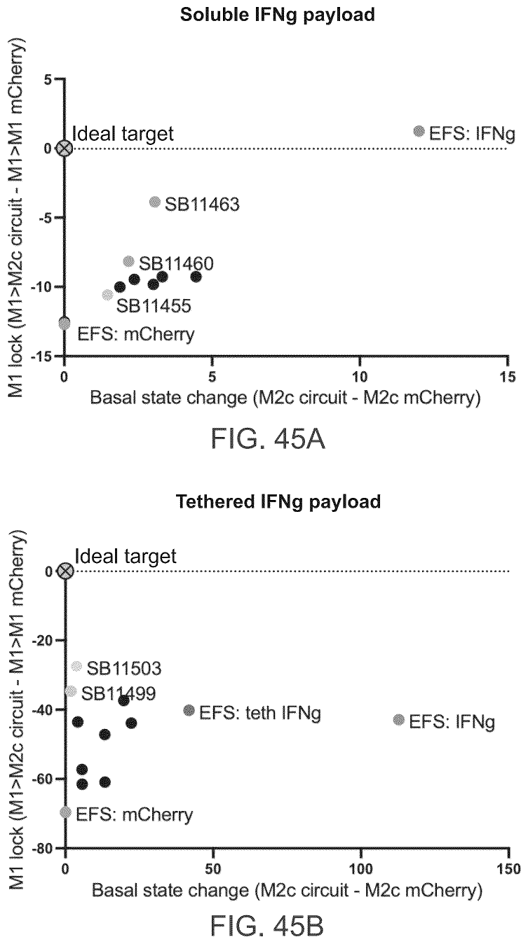

FIGS. 45A and 45B depict TNFalpha output in the M1→M2c state plotted against TNFalpha output in the M2c state, for the candidate soluble IFN-γ circuits (FIG. 45A) and the candidate M1-tethered IFN-γ circuits (FIG. 45B). The x-axis was calculated by subtracting the TNFa production (as measured by Luminex) in the M2c state of the negative control circuit (EFS:mCherry) from the M2c TNFa production of the circuit of interest. The y-axis was calculated by subtracting the TNFa production in the late M1 time point (M1 to M1 polarization state) of the negative control circuit (EFS:mCherry) from the TNFa production in the transpolarized (M1→M2c) state of the circuit of interest. The x-axis therefore represents the change in TNFa caused by the circuit in the basal state (ideal circuits will keep this number low). The y-axis represents the change in TNFa caused by the circuit in the transpolarized state relative to the M1 state. The higher the y-value, the greater lock capability the circuit has. When plotted this way, we see that SB11463 and SB11503 exhibited an optimal combination of M1 phenotype locking ability with low basal activation for the soluble IFNg and tethered IFNg payloads, respectively. SB11499 was also a promising circuit for the tethered IFNg payload.

### Example 16: Testing of M2 Phenotype Switch Circuit Pairing M2c Promoters with IRF7

Lead M2c promoters are paired with the M1 master regulator IRF7. M2c promoter: IRF7 constructs are packaged into vpx lentivirus and transduced into macrophages at an MOI of 5. On day 0, monocytes are isolated from PBMCs and macrophage differentiation is initiated by addition of M-CSF ot the media. On day 4, macrophages are plated into 96 well plates at 80,000 cells per well. On day 5, macrophages are transduced with the M1 pro: IRF7 or IRF7-PEST constructs. On day 7, for each construct tested, macrophages are polarized to M0, M1, or M2c. On day 9, M0, M1, and M2c cells are analyzed by flow cytometry and supernatants are harvested for Luminex analysis. All cytokine and flow markers are analyzed one by one for the impact of the IRF7 and IRF7-PEST circuits and also are analyzed together by principal component analysis.

### Example 17: Testing of M2 Phenotype Switch Circuit Pairing M2c Promoters with IRF7

Selected M2c promoters were paired with the M1 master regulator IRF7 linked to the PEST degron to decrease the half-life of the IRF7 transcription factor (reducing off-target activation due to potentially leaky IRF7 expression in the M0 and M2c states as a result of transient M1-like activation during lentiviral transduction). Constructs used in two experiment rounds described in this Example are shown below.

| SB ID | Promoter | Regulator | Condition | EXP-Round |
|---|---|---|---|---|
| SB07683 | EFS | mCherry | ”−” Control | Both |
| | | | | Experiments |
| SB12957 | EFS | Tethered | “+” Phenotype | Round 1 only |
| | | IFNg | Control | |
| SB09073 | EFS | Soluble | “+” Phenotype | Round 2 only |
| | | IFNg | Control | |
| SB12980 | EFS | IRF7-PEST | Constitutive | Both |
| | | | Control | Experiments |
| SB12981 to | Top M2 | IRF7-PEST | — | Both |
| SB12991 | Promoter | | | Experiments |
| | Candidates | | | |
| | driving | | | |
| | phenotype | | | |
| | switch | | | |
| SB12992 | EFS | IRF7 | Constitutive | Both |
| | | | Control | Experiments |

Sequences for IRF7-PEST are shown in Table 7.

M2c pro:IRF7-PEST constructs were packaged into vpx lentivirus. Cell processing, transductions, and polarizations were performed as described below.

On day 0, monocytes were isolated from PBMCs and macrophage differentiation began by addition of M-CSF to the media. On day 4, macrophages were plated into 96 well plates at 80,000 cells per well. On day 5, macrophages were transduced with the applicable constructs. On day 6, for each construct tested, macrophages were polarized to M0, M1, or M2c. On day 8, M0, M1, and M2c cells were analyzed by flow cytometry and supernatants were harvested for Luminex analysis.

All cytokine and flow markers were analyzed one by one for the impact of the tested constructs and also were analyzed together by principal component analysis.

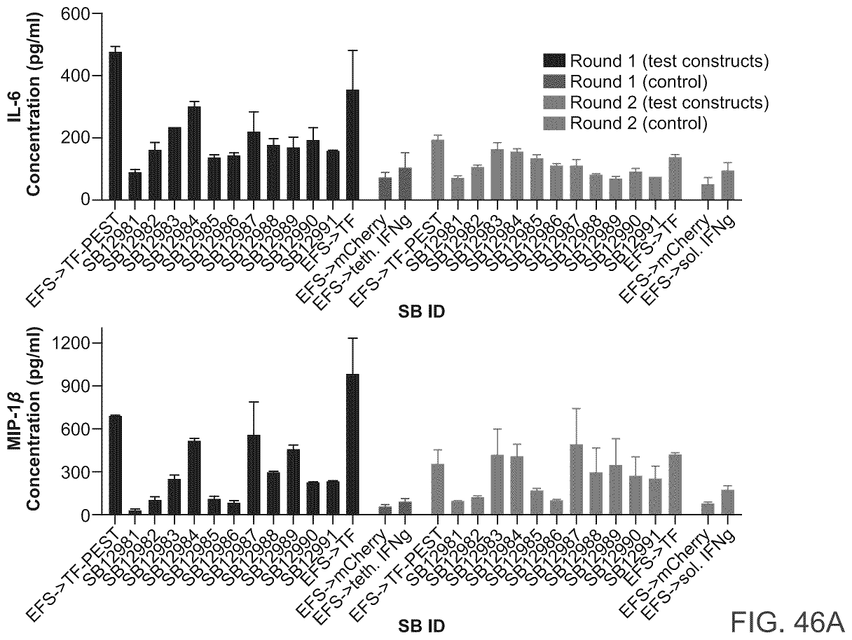

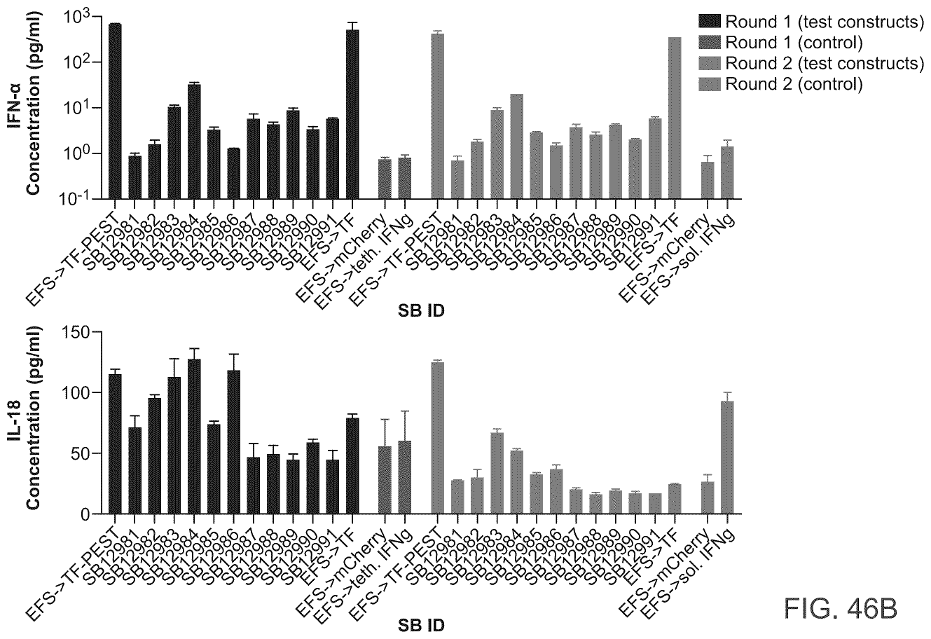

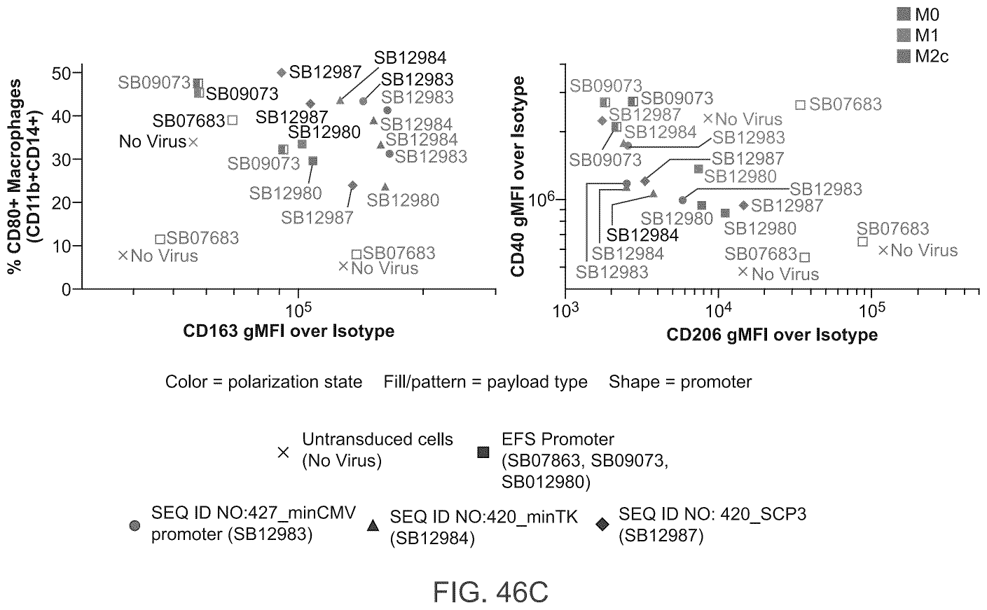

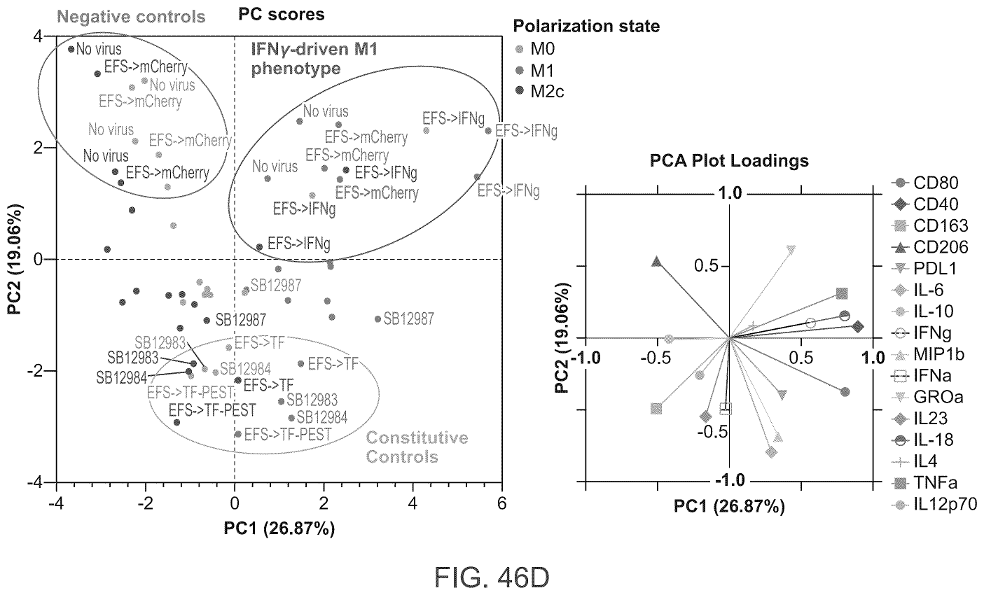

Results for IL-6 and MIP-1β M1-associated markers in cells subjected to M2c polarizing conditions are shown in FIG. 46A. Results for INF-α and IL-18 M1-associated markers in cells subjected to M2c polarizing conditions are shown in FIG. 46B. As shown, SB12983 (same promoter as SB11754, comprising the enhancer sequence SEQ ID NO: 427 linked to minCMV), SB12984 (same promoter as SB11755, comprising the enhancer sequence SEQ ID NO: 420 linked to minTK minimal promoter), and SB12987 (same promoter as SB11771, comprising the enhancer sequence SEQ ID NO: 420 linked to SCP3 minimal promoter) exceeded the EFS→IFNg benchmark in driving both IL-6 and MIP-1(3 expression, and SB12983 and SB12984 exceeded the EFS→IFNg benchmark in driving IFNα and IL-18 expression. FIG. 46C depicts flow results for the M1 marker CD80 and M2 marker CD163,in transduced macrophages subjected to M0, M1, and M2c polarization states. FIG. 46D depicts PCA analysis of 15 markers: 4 flow surface markers (CD80, CD40, CD163, CD206, PDL1) and 11 cytokines (IL-6, IL-10, IFNγ, MIP-1β, IFNα, GROα, IL-23, IL-18, IL-19, IL-4, TNFα, IL-12p70). Principal component plot is shown on the left and loadings vectors are shown on the right. Results indicate that SB09073 (EFS-soluble IFNg) and SB12980 (EFS-IRF7-PEST) constructs drove cells toward an M1-liked state, and that phenotype “switch” circuits SB12983, SB12984, and SB12987 performed comparably to benchmarks, while exhibiting some leaky M1 activity in the M0 state. These results suggest that these constructs can be used to induce an M2→M1 phenotype switch. Furthermore, the constitutively expressed IRF7-PEST payload (SB12980) had comparable performance to the non-PEST tagged IRF7 payload (SB12992), suggesting that IRF7-PEST maintains high levels of activity in driving phenotype changes despite having higher protein turnover. The IRF7-PEST payload exhibited long-lasting phenotype effects despite being engineered for fast degradation.

### Interpretations

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An engineered macrophage-specific promoter system comprising:
a. a regulatory element, wherein the regulatory element is derived from a promoter of a gene selected from the group consisting of CCL19, CCR7, CXCL11, GBP5, IDO1, UBD, and UNQ6494.1; and
b. a heterologous payload, optionally wherein the heterologous payload is selected from the group consisting of transcriptions factors, cytokines, receptors, enzymes, chemokines, antibodies, fragments of antibodies, miRNAs, and shRNAs, wherein the regulatory element exhibits greater activity in an M1 macrophage compared to an M2 or M0 macrophage, and wherein the regulatory element is or comprises an enhancer region that is derived from a promoter of a gene that is more highly expressed in M1 macrophage compared to M2 or M0 macrophages,
optionally wherein M2 macrophages are selected from the group consisting of M2a macrophages, M2b macrophages, and M2c macrophages, optionally wherein the regulatory element comprises a sequence having at least 75%, at least 80%, at least 85%, at least 90%, at least 95%, at least 96%, at least 97%, at least 98%, at least 99% or 100% sequence identity to a nucleotide sequence selected from the group consisting of SEQ ID NO: 132-138.

2. An engineered macrophage-specific promoter system comprising:
a. a regulatory element, wherein the regulatory element is derived from a promoter of a gene selected from the group consisting of CD28, SOCS3, PLXDC1, IL7R ZNF704, LNCAROD, MRC1, and ID3; and
b. a heterologous payload, optionally wherein the heterologous payload is selected from the group consisting of transcriptions factors, cytokines, receptors, enzymes, chemokines, antibodies, fragments of antibodies, miRNAs, and shRNAs,
wherein the regulatory element exhibits greater activity in an M2 macrophage compared to an M1 or M0 macrophage, and wherein the regulatory element is or comprises an enhancer region that is derived from a promoter of a gene that is more highly expressed in M2 macrophage compared to M1 or M0 macrophages, optionally wherein M2 macrophages are selected from the group consisting of M2a macrophages, M2b macrophages, and M2c macrophages, optionally wherein the regulatory element comprises a sequence having at least 75%, at least 80%, at least 85%, at least 90%, at least 95%, at least 96%, at least 97%, at least 98%, at least 99% or 100% sequence identity to a nucleotide sequence selected from the group consisting of SEQ ID NO: 139-141, 392, 393, and 414-419.

3. An engineered macrophage-specific promoter comprising an ablation of at least one nucleotide motif, wherein the ablation increases specific activity of the engineered macrophage-specific promoter in M1 macrophages, as compared to activity of a corresponding macrophage-specific promoter lacking the ablation in M1 macrophages, optionally wherein the corresponding macrophage-specific promoter lacking the ablation in M1 macrophages is a wildtype macrophage promoter, and wherein the wildtype macrophage promoter comprises a sequence selected from the group consisting of SEQ ID NOs: 132-138, wherein the engineered macrophage-specific promoter comprises:
i) a motif within the nucleotide sequence of SEQ ID NO: 132, wherein the motif comprises a sequence selected from the group consisting of: position 63 to position 73 of SEQ ID NO: 132, position 80 to position 102 of SEQ ID NO: 132, position 141 to position 162 of SEQ ID NO: 132, position 212 to position 222 of SEQ ID NO: 132, position 229 to position 251 of SEQ ID NO: 132, position 307 to position 361 of SEQ ID NO: 132, position 365 to position 376 of SEQ ID NO: 132, position 559 to position 571 of SEQ ID NO: 132, position 617 to position 633 of SEQ ID NO: 132, position 782 to position 799 of SEQ ID NO: 132, position 852 to position 871 of SEQ ID NO: 132, position 886 to position 920 of SEQ ID NO: 132, position 933 to position 959 of SEQ ID NO: 132, position 1002 to position 1028 of SEQ ID NO: 132, position 1032 to position 1045 of SEQ ID NO: 132, position 1064 to position 1087 of SEQ ID NO: 132, position 1169 to position 1192 of SEQ ID NO: 132, position 1212 to position 1232 of SEQ ID NO: 132, position 1257 to position 1275 of SEQ ID NO: 132, position 1310 to position 1333 of SEQ ID NO: 132, position 1381 to position 1434 of SEQ ID NO: 132, position 1698 to position 1753 of SEQ ID NO: 132, position 1783 to position 1826 of SEQ ID NO: 132, position 1909 to position 1927 of SEQ ID NO: 132, position 1946 to position 1961 of SEQ ID NO: 132; and/or
ii) a motif within the nucleotide sequence of SEQ ID NO: 136, wherein the motif comprises a sequence selected from the group consisting of: to position 133 to position 144 of SEQ ID NO: 136, position 200 to 217 of SEQ ID NO: 136, position 225 to position 247 of SEQ ID NO: 136, position 303 to position 325 of SEQ ID NO: 136, position 332 to position 342 of SEQ ID NO: 136, position 391 to position 413 of SEQ ID NO: 136, position 423 to position 460 of SEQ ID NO: 136, position 467 to position 477 of SEQ ID NO: 136, position 693 to position 717 of SEQ ID NO: 136, position 738 to position 761 of SEQ ID NO: 136, position 838 to position 861 of SEQ ID NO: 136, position 1229 to position 1246 of SEQ ID NO: 136, position 1286 to position 1309 of SEQ ID NO: 136, position 1413 to position 1431 of SEQ ID NO: 136, position 1456 to position 1473 of SEQ ID NO: 136, to position 1530 to position 1544 of SEQ ID NO: 136, position 1577 to position 1590 of SEQ ID NO: 136, position 1816 to position 1836 of SEQ ID NO: 136, position 1852 to position 1872 of SEQ ID NO: 136, and to position 1876 to position to position 1896 of SEQ ID NO: 136; and/or
iii) a motif within the nucleotide sequence of SEQ ID NO: 137, wherein the motif comprises a sequence selected from the group consisting of: to position 43 to position 60, position 107 to position 120 of SEQ ID NO: 137, position 210 to position 230 of SEQ ID NO: 137, position 345 to position 407 of SEQ ID NO: 137, position 427 to position 457 of SEQ ID NO: 137, position 468 to position 484 of SEQ ID NO: 137, position 560 to position 582, position 730 to position 746 of SEQ ID NO: 137, position 809 to position 820 of SEQ ID NO: 137, position 827 to position 837 of SEQ ID NO: 137, position 858 to position 878 of SEQ ID NO: 137, position 1291 to position 1302 of SEQ ID NO: 137, position 1321 to position 1341 of SEQ ID NO: 137, position 1435 to position 1463 of SEQ ID NO: 137, position 1530 to position 1541 of SEQ ID NO: 137, position 1707 to position 1718 of SEQ ID NO: 137, position 1834 to position 1863 of SEQ ID NO: 137, position 1870 to position 1882 of SEQ ID NO: 137, and to position 1913 to position 1929 of SEQ ID NO: 137,
optionally wherein the ablation comprises a substitution or deletion of one or more nucleotides of the at least one nucleotide motif.

4. An engineered macrophage-specific promoter comprising at least one regulatory element, wherein the regulatory element exhibits greater activity in an M1 macrophage compared to an M2 or M0 macrophage or exhibits greater activity in an M2 macrophage compared to an M1 or M0 macrophage, optionally wherein the engineered macrophage-specific promoter comprises at least 2, at least 3, at least 4, or at least 5 regulatory elements, optionally wherein each of the regulatory elements are the same or different, optionally wherein M2 macrophages are selected from the group consisting of M2a macrophages, M2b macrophages, and M2c macrophages.

5. The engineered macrophage-specific promoter of any one of claims 1-4, wherein the at least one regulatory element comprises a nucleotide sequence selected from:
i) a nucleotide sequence having at least 75%, at least 80%, at least 85%, at least 90%, at least 95%, at least 96%, at least 97%, at least 98%, at least 99% or 100% sequence identity to SEQ ID NO: 297-313;
ii) a nucleotide sequence having at least 75%, at least 80%, at least 85%, at least 90%, at least 95%, at least 96%, at least 97%, at least 98%, at least 99% or 100% sequence identity to SEQ ID NO: 372-390;
iii) a nucleotide sequence having at least 75%, at least 80%, at least 85%, at least 90%, at least 95%, at least 96%, at least 97%, at least 98%, at least 99% or 100% sequence identity to SEQ ID NO: 440-443,
iv) a nucleotide sequence having at least 75%, at least 80%, at least 85%, at least 90%, at least 95%, at least 96%, at least 97%, at least 98%, at least 99% or 100% sequence identity to SEQ ID NOs: 314-371,
v) a nucleotide sequence having at least 75%, at least 80%, at least 85%, at least 90%, at least 95%, at least 96%, at least 97%, at least 98%, at least 99% or 100% sequence identity to SEQ ID NO: 420-439,
optionally wherein the engineered macrophage-specific promoter further comprises a minimal promoter operably linked to the engineered macrophage-specific promoter, optionally wherein the minimal promoter is derived from a promoter selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, inducer molecule responsive promoters, CMV, EFS, SFFV, SV40, MND, PGK, UbC, hEFlaV1, hCAGG, hEFlaV2, hACTb, heIF4A1, hGAPDH, hGRP78, hGRP94, hHSP70, hKINb, hUBIb, and tandem repeats thereof.

6. An engineered macrophage-specific promoter comprising at least one regulatory element comprising a nucleotide sequence having at least 75%, at least 80%, at least 85%, at least 90%, at least 95%, at least 96%, at least 97%, at least 98%, at least 99% or 100% sequence identity to SEQ ID NO: 1-29, 81-82, 88-97, 119-122, 132-138, 142-163, 97-313, 139-141, 314-371, 390, 392-393, and 420-443, optionally wherein the regulatory element or the engineered macrophage-specific promoter is operably linked to a minimal promoter, wherein optionally the minimal promoter comprises a sequence of a promoter selected from minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, SCP3, YB-SCP3, inducer molecule responsive promoters, CMV, EFS, SFFV, SV40, MND, PGK, UbC, hEFlaV1, hCAGG, hEFlaV2, hACTb, heIF4A1, hGAPDH, hGRP78, hGRP94, hHSP70, hKINb, hUBIb, and tandem repeats thereof, optionally wherein the engineered macrophage-specific promoter system further comprises a translation initiator site, optionally wherein the translation initiator site is or comprises a Kozak sequence.

7. The engineered macrophage-specific promoter system of claim 1 or 3, wherein the regulatory element or the engineered macrophage-specific promoter comprises:
a) a first transcriptional activating element as set forth in SEQ ID NO: 220, a second transcriptional activating element as set forth in SEQ ID NO: 222, a third transcriptional activation element as set forth in SEQ ID NO: 240, a fourth transcriptional activating element as set forth in SEQ ID NO: 254, and a fifth transcriptional activating element as set forth in SEQ ID NO: 256; and does not comprise at least one repressive element selected from: SEQ ID NO: 226, SEQ ID NO: 234, SEQ ID NO: 236, SEQ ID NO: 238, SEQ ID NO: 246, and SEQ ID NO: 252, optionally further comprising a sixth transcriptional activating element as set forth in SEQ ID NO: 224 and/or a seventh transcriptional activating element as set forth in SEQ ID NO: 258, optionally wherein the regulatory element or the engineered macrophage-specific promoter further do not comprise SEQ ID NO: 228, SEQ ID NO: 230, SEQ ID NO: 232, SEQ ID NO: 242, SEQ ID NO:244, SEQ ID NO: 248, and SEQ ID NO: 250, optionally wherein the regulatory element or the engineered macrophage-specific promoter does not comprise the repressive elements as set forth in SEQ ID NO: 226, SEQ ID NO: 236, SEQ ID NO: 238, SEQ ID NO: 246, and SEQ ID NO: 252, optionally wherein the regulatory element or the engineered macrophage-specific promoter comprises: a sequence as set forth in GTTAAGTGGCTAGGGATAACATTGAGGCACTAAAGCATTATTGGTTCTGC AGTCAAGGGTAGGATAGATTGTTTTTTTTTTTTT (SEQ ID NO: 482), and a sequence as set forth in TTTGTGGTTTTATTGGTTTTCATATTACAAACAAAGAAACTAGAAAATGAA ACCATTCCAAAAGTGGAAGTAATTTCTCA (SEQ ID NO: 483), or a sequence as set forth in GCTCTTCTAAAAATATGCGAAATGAGGTTTTTAGGGAGGTGTAGGTATGG CTGAAGAAAATCAAGGTGAATGAAGACAAGATCAATTGAGAATGTAGTT TCAGAAATAGCAAAGAAGCCAAAGTTTGAGGAAGTTAAGTGGCTAGGGA TAACATTGAGGCACTAAAGCATTATTGGTTCTGCAGTCAAGGGTAGGATA GATTGTTTTTTTTTTTTTTGAGACGGAGTCTCACTCTGCTGCCCAGGC (SEQ ID NO: 484), a sequence as set forth in ATTTTGGTTTCAGTTTTCCTTAC (SEQ ID NO: 240), and a sequence as set forth in TTTGTGGTTTTATTGGTTTTCATATTACAAACAAAGAAACTAGAAAATGAA ACCATTCCAAAAGTGGAAGTAATTTCTCA (SEQ ID NO: 483); or
b) a first transcriptional activating element as set forth in SEQ ID NO: 268 and a second transcriptional activating element as set forth in SEQ ID NO: 270 and does not comprise at least one repressive element selected from: SEQ ID NO: 260, SEQ ID NO: 262, SEQ ID NO: 264, SEQ ID NO: 266, SEQ ID NO: 272, and SEQ ID NO: 391; or at least one, at least two, at least three, at least four, or at least five tandem repeats of SEQ ID NO: 268 and SEQ ID NO: 270;
optionally further comprising a third transcriptional activating element as set forth in SEQ ID NO: 291 and/or a fourth transcriptional activating element as set forth in: SEQ ID NO: 295,
optionally wherein the regulatory element or the engineered macrophage-specific promoter does not comprise the repressive elements as set forth in SEQ ID NO: 262, SEQ ID NO: 264, SEQ ID NO: 272, and SEQ ID NO: 391, optionally wherein the regulatory element or the engineered macrophage-specific promoter further does not comprise SEQ ID NO: 260 and/or SEQ ID NO: 266.

8. A heterologous construct comprising
i) the engineered macrophage-specific promoter system of claim 1 or 2; or
ii) the engineered macrophage-specific promoter of any one of claims 3-7 operably linked to a heterologous payload, optionally wherein the heterologous payload is a polynucleotide comprising a nucleotide sequence encoding a polypeptide, optionally wherein the polypeptide comprises at least one effector molecule,
optionally wherein the polypeptide comprises a first effector molecule and a second effector molecule,
optionally wherein the engineered macrophage specific promoter comprises a regulatory element selected from: a nucleotide sequence having at least 75%, at least 80%, at least 85%, at least 90%, at least 95%, at least 96%, at least 97%, at least 98%, at least 99% or 100% sequence identity to SEQ ID NO: 420; and a nucleotide sequence having at least 75%, at least 80%, at least 85%, at least 90%, at least 95%, at least 96%, at least 97%, at least 98%, at least 99% or 100% sequence identity to SEQ ID NO: 427, optionally wherein the polynucleotide comprises a nucleotide sequence encoding the first effector molecule, a linker nucleotide sequence, and a nucleotide sequence encoding the second effector, optionally wherein the linker nucleotide sequence encodes one or more 2A ribosome skipping elements, optionally wherein the one or more 2A ribosome skipping elements comprise elements that are each selected from the group consisting of: P2A, T2A, E2A, and F2A.

9. The heterologous construct of claim 8, wherein the at least one effector molecule or each effector molecule is selected from a therapeutic class, wherein the therapeutic class is selected from the group consisting of: a cytokine, a chemokine, a homing molecule, a growth factor, a polynucleotide molecule, a co-activation molecule, a tumor microenvironment modifier, a receptor, a ligand, a transcription factor, an antibody, a peptide, and an enzyme, optionally wherein the transcription factor is a master regulator, optionally wherein the transcription factor is a master regulator of polarization to an M1 macrophage, optionally wherein the transcription factor is IRF7 or a derivative thereof, or p65/RelA or a derivative thereof, optionally wherein the transcription factor is a master regulator of polarization to an M2 macrophage, optionally wherein the at least one effector molecule or each effector molecule is or comprises a cytokine, chemokine, homing molecule, growth factor, a tumor microenvironment modifier, co-optionally wherein the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha, optionally wherein the cytokine is a master regulator of polarization to an M1 macrophage, optionally wherein the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof, optionally wherein the cytokine is a master regulator of polarization to an M2 macrophage, optionally wherein the cytokine is IL-10, IL-4, IL-13, IL-21, TGF-beta, M-CSF, or a derivative thereof, optionally wherein the chemokine is selected from the group consisting of: CCL21a, CXCL10, CXCL11, CXCL13, a CXCL10-CXCL11 fusion protein, CCL19, CXCL9, and CXCL1, optionally wherein the homing molecule is selected from the group consisting of: anti-integrin alpha4, beta7; anti-MAdCAM; CCR9; CXCR4; SDF1; MMP-2; CXCR1; CXCR7; CCR2; CCR4; and GPR15, optionally wherein the growth factor is selected from the group consisting of: FLT3L and GM-CSF, optionally wherein the co-activation molecule is selected from the group consisting of: c-Jun, 4-1BBL and CD40L, optionally wherein the tumor microenvironment modifier is selected from the group consisting of: an adenosine deaminase, a TGFbeta inhibitor, an immune checkpoint inhibitor, a VEGF inhibitor, and an HPGE2, optionally wherein each of the first effector molecule and the second effector molecule are from separate therapeutic classes, optionally wherein each effector molecule is a human-derived effector molecule, optionally wherein the cytokine is modified to comprise a membrane tethering domain, optionally wherein the membrane tethering domain is or comprises a transmembrane-intracellular domain and/or transmembrane domain of a protein selected from: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, and BTLA, or a functional portion thereof, optionally wherein the master regulator of polarization to an M1 macrophage is IRF7 or a derivative thereof, optionally wherein the derivative of IRF7 comprises IRF7 operably linked to a degron domain, optionally wherein the degron domain is selected from: a PEST domain, HCV NS4 degron, GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 190) phospho-dependent degron, an Siah binding motif, an SPOP SBC docking motif, a PCNA binding PIP box, and derivatives thereof, optionally wherein the degron domain is a PEST domain, optionally wherein the PEST comprises the amino acid sequence SEQ ID NO: 501 or a derivative thereof.

10. A heterologous construct for inducing a macrophage to transition from an M1 state to an M2 state, comprising:
either
i) the regulatory element derived from a promoter of a gene that is more highly expressed in M1 macrophage compared to M2 or M0 macrophages, as applied to claim 1, or
ii) the engineered macrophage-specific promoter of any one of claims 3-7; and
a heterologous payload encoding a master regulator of polarization to an M2 macrophage,
wherein the regulatory element or engineered macrophage-specific promoter of (a) is operably linked to the heterologous payload and configured to induce expression of the heterologous payload, optionally wherein the master regulator of polarization to an M2 macrophage is IL-10, IL-4, IL-13, IL-21, TGF-beta, M-CSF, or a derivative thereof, optionally wherein the master regulator of polarization to an M2 macrophage is IL-10, optionally wherein the M2 state is an M2c state, an M2a state, or an M2b state, optionally wherein (a) is a regulatory element derived from a CCL19 promoter, optionally comprising the nucleotide sequence of SEQ ID NO: 132.

11. A heterologous construct for stabilizing a macrophage in an M1 polarization state, comprising:
either
i) the regulatory element derived from a promoter of a gene that is more highly expressed in M1 macrophage compared to M2 or M0 macrophages, optionally wherein the regulatory element derived from a UBD1 promoter, an IDO1 promoter, or a CCL19 promoter, as applied to claim 2, or
ii) the engineered macrophage-specific promoter of any one of claims 3-7; and a heterologous payload encoding a master regulator of polarization to an M1 macrophage,
wherein the regulatory element or engineered macrophage-specific promoter of (a) is operably linked to the heterologous payload and configured to induce expression of the heterologous payload, optionally wherein the master regulator of polarization to an M1 macrophage is a cytokine, optionally wherein the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof, optionally wherein the master regulator of polarization to an M1 macrophage is a transcription factor selected from IRF7 or a derivative thereof, or p65/RelA or a derivative thereof.

12. A heterologous construct for inducing a macrophage to transition from an M2 state to an M1 state, comprising:
either
i) the regulatory element derived from a promoter of a gene that is more highly expressed in M2 macrophage compared to M1 or M0 macrophages, as applied to claim 2, or
ii) the engineered macrophage-specific promoter of any one of claims 3-7; and a heterologous payload encoding a master regulator of polarization to an M1 macrophage,
wherein the regulatory element or engineered macrophage-specific promoter of (a) is operably linked to the heterologous payload and configured to induce expression of the heterologous payload, optionally wherein the master regulator of polarization to an M1 macrophage is a cytokine, optionally wherein the cytokine is IFNgamma, IFNalpha, TNF alpha, GM-CSF, IL-12, IL-12p70, IL-12p40, IL-12p35, IL-6, IL-23, IL-1alpha, IL-1beta, or a derivative thereof, optionally wherein the master regulator of polarization to an M1 macrophage is a transcription factor selected from IRF7 or a derivative thereof, or p65/RelA or a derivative thereof.

13. A heterologous construct for stabilizing a macrophage in an M2 polarization state, comprising:
either
i) the regulatory element derived from a promoter of a gene that is more highly expressed in M2 macrophage compared to M1 or M0 macrophages, as applied to claim 2, or
ii) the engineered macrophage-specific promoter of any one of claims 3-7; and a heterologous payload encoding a master regulator of polarization to an M2 macrophage,
wherein the regulatory element or engineered macrophage-specific promoter of (a) is operably linked to the heterologous payload and configured to induce expression of the heterologous payload, optionally wherein the M2 state is an M2c state, an M2a state, or an M2b state, optionally wherein the master regulator of polarization to an M2 macrophage is IL-10, IL-4, IL-13, IL-21, TGF-beta, M-CSF, or a derivative thereof.

14. A vector comprising the heterologous construct according to any one of claims 8-13.

15. A dual expression vector comprising the heterologous construct according to claim 14 and a second construct comprising a nucleotide sequence encoding an activating immune receptor.

16. An immunoresponsive cell comprising the heterologous construct according to any one of claims 8-13, the vector according to claim 14, or the dual expression vector according to claim 15, optionally wherein the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell, optionally wherein the immunoresponsive cell is a macrophage, optionally wherein the macrophage is a tumor-resident macrophage, optionally wherein the immunoresponsive cell is autologous or allogeneic, optionally wherein the immunoresponsive cell expresses an activating immune receptor, optionally wherein the activating immune receptor comprises an antigen recognizing receptor.

17. A pharmaceutical composition comprising the vector of claim 14, the dual expression vector according to claim 15, or the immunoresponsive cell according to claim 16, and a pharmaceutically acceptable carrier, pharmaceutically acceptable excipient, or a combination thereof.

18. A method of increasing expression of a target gene, the method comprising use of the engineered macrophage-specific promoter of any one of claims 1-7, the vector of claim 14, or the dual expression vector according to claim 15 to increase expression of the target gene, optionally wherein the target gene is an immunomodulatory gene.

19. A method of treating a subject in need thereof, the method comprising administering a therapeutically effective dose of the vector of claim 14, the dual expression vector according to claim 15, the immunoresponsive cell according to claim 16, or the pharmaceutical composition according to claim 17.

20. A kit for treating and/or preventing a disease or disorder, comprising the immunoresponsive cell according to any one of claim 16 or a pharmaceutical composition according to claim 17, optionally wherein the kit further comprises written instructions for using the immunoresponsive cell for treating and/or preventing a disease or disorder in a subject, optionally wherein the disease is cancer.
